(19) United States
(12) Patent Application Publication
(10) Pub. No.: US 2025/0122192 A1
(43) Pub. Date: Apr. 17, 2025

(54) 5-PYRIMIDINECARBOXAMIDE DERIVATIVES AND METHODS OF USING THE SAME

(71) Applicant: Rgenta Therapeutics, Inc., Woburn, MA (US)

(72) Inventors: Travis T. Wager, Brookline, MA (US); Zhiping Weng, Wellesley, MA (US); Hualin Simon Xi, Lexington, MA (US)

(21) Appl. No.: 18/714,624

(22) PCT Filed: Dec. 2, 2022
(86) PCT No.: PCT/US2022/051722

§ 371 Date: May 30, 2024

Related U.S. Application Data
(60) Provisional application No. 63/285,629, filed on Dec. 3, 2021.

(51) Int. Cl. C07D 471/04; A61K 31/506; A61K 31/5383; A61K 31/5386; A61K 31/55; A61K 31/551; C07D 401/14; C07D 403/12; C07D 413/12; C07D 487/04; C07D 519/00

(52) U.S. Cl. CPC C07D 471/04; A61K 31/506; A61K 31/5383; A61K 31/5386; A61K 31/55; A61K 31/551; C07D 401/14; C07D 403/12; C07D 413/12; C07D 487/04; C07D 519/00

(57) ABSTRACT

The present disclosure relates to compounds of Formula (I); and to their prodrugs, pharmaceutically acceptable salts, pharmaceutical compositions, methods of use, and methods for their preparation. The compounds of the present disclosure may act as small molecule splicing modulator compounds that modulate splicing of mRNA, such as pre-mRNA, encoded genes, and methods of use of the compounds for modulating splicing and treating related diseases and conditions. The compounds disclosed herein may possess activity toward various genetic pathways and are accordingly useful in methods of treatment of the human or animal body.

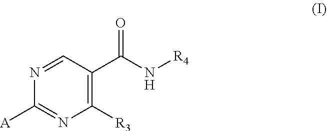

### CROSS REFERENCE TO RELATED APPLICATIONS

This application claims priority to and the benefit of U.S. Provisional Application No. 63/285,629, filed Dec. 3, 2021, the contents of which are hereby incorporated by reference in their entirety.

### BACKGROUND

More than 90% of human genes produce multiple mature transcripts via More than 90% of human genes produce multiple mature transcripts via alternative splicing. This process is essential for generating different transcripts in different cell and tissue types, during the developmental process, and in response to internal and external signals. Alternative splicing are prevalent not only for protein-coding genes but also for most other kinds of genes including microRNA genes and long noncoding genes. Splicing is carried out by the spliceosome. Small nuclear RNAs (snRNAs) are key components of the spliceosome. The major spliceosome comprises the U1, U2, U4, U5, and U6 snRNAs, and it catalyzes the removal of ˜95% of human introns, while the remaining introns (called the U12-type of introns) are removed by the minor spliceosome, comprising the U11, U12, U4atac, U5, and U6atac snRNAs. These snRNAs are in complex with their respective protein partners to form the functional unit of small nuclear ribonucleoproteins (snRNPs).

Splicing is a highly regulated process, with the regulation exerted by both cis-elements and trans-factors. The cis-elements that are recognized by the snRNAs include the 5′-splice site, 3′-splice site, and the branchpoint, each of these associating with a sequence motif that is recognized by a component of the spliceosome. In addition, there are intronic splicing enhancers (ISE), intronic splicing silencer (ISS), exonic splicing enhancer (ESE), and exonic splicing enhancer (ESS), which are recognized by a myriad of trans-factors commonly known as RNA-binding proteins (RBPs). Some of these RBPs directly bind to the cis-elements in a sequencing-specific way, while other RBPs recognize RNA structures (e.g., RNA duplex or unpaired loop region), yet others function via protein-protein interaction. There are ˜1600 RBPs annotated in the human genome, and they are expressed in a cell-type-specific manner and form an extensive regulatory network for splicing regulation.

Dysregulation of splicing is implicated in roughly half of human diseases. Some diseases are caused by mutations in the spliceosome components or RBPs, while others by mutations in the cis-elements such as splice sites, branchpoint, or the various splicing enhancers and silencers. Although current approaches to treating these diseases, such as CRISPR-based genome editing, virus-aided gene therapy, or a variety of oligonucleotide-based technologies, continue to improve, they still suffer major technical and clinical challenges. In particular, oligonucleotide-based therapeutics show unfavorable pharmacokinetics, can not be orally administered, and can not be delivered effectively to many tissues, especially the brain. Small-molecule drugs have excellent pharmacokinetics, effective delivery, and bioavailability, and have only recently become available for modulating RNA splicing. Yet, the currently available molecules come from a few limited chemical series. Thus, there is a great need to develop additional small molecule splicing modulators (SMSMs).

Almost 50 inherited disorders in humans result from an increase in the number of copies of single repeats in genomic DNA. These DNA repeats appear to be predisposed to such expansion because they have unusual structural characteristics, which disrupt cellular replication, repair, and recombination machinery. The presence of expanding DNA repeats alters gene expression in human cells, leading to disease.

One of these inherited disorders is Huntington's disease (HD). HD is a deadly neurodegenerative disorder with no cure associated with cognitive impairment, dementia, and loss of motor coordination. It is characterized by the progressive and hereditary increase in the length of the CAG trinucleotide repeats that encode a stretch of polyglutamine, in the Huntington gene (HTT) coding region. These repeats can increase in number from one generation to the next. The normal allele of the HTT gene contains fewer than 36 CAG repeats, while the mutant allele contains more than 36 repeats. Most HD patients carry one normal allele and one mutant allele that causes the disease. Functionally, the aberrant accumulation of CAG repeats is believed to confer a toxic gain of function on the mutant HD protein, causing it to aggregate, form protein deposits (i.e., inclusion bodies), and induce cell death. The severity of the disease generally reflects the extent of repeat expansion in the mutant HTT protein.

Myotonic dystrophy type 1 (DM1) and type 2 (DM2) are associated with long repeats of polyCUG and polyCCUG in the 3′-UTR and intron 1 regions of the transcription of myotonic dystrophy protein kinase (DMPK) and protein 9 of zinc finger (ZNF9), respectively. While normal individuals have up to 30 CTG repeats, DMI patients have a higher number of repeats ranging from 50 to thousands. The severity of the disease and the age of onset correlate with the number of repetitions. Adult-onset patients show milder symptoms and have fewer than 100 repeats, juvenile-onset DM1 patients have up to 500 repeats, and congenital cases typically have around 1,000 CTG repeats. Expanded transcripts containing CUG repeats form a secondary structure, accumulate in the nucleus as nuclear foci, and sequester RNA-binding proteins (RNA-BP).

Besides the extra copies of repeats inherited at birth, for many repeat expansion diseases, repeats are highly unstable and their repeat numbers continue to expand throughout the life time of patients. This repeat instability has been shown experimentally to be mediated by proteins in DNA mismatch repair (MMR) processes including PMS1, MLH1, MSH3. Human genetics data from genome-wide association studies has indicated that variants in MMR proteins are associated with clinically relevant HD symptomatology including age at motor onset, rate of progression and somatic instability. Knocking down and knocking out MMR genes have been shown to stall or slow the somatic repeat expansion in various preclinical models of repeat expansion diseases. Thus, small molecule splicing modulators of MMR genes would be useful therapeutic agents to treat a variety of repeat expansion diseases.

### SUMMARY

Here we describe a series of novel small molecule splicing modulators (SMSMs), which can be used to treat a wide variety of diseases, including neurodegenerative and repeat expansion diseases. These SMSMs target regions of a primary RNA transcript that are cis-elements, such as splice sites, branch points, splicing enhancers, or splicing silencers. These regions may contain unpaired nucleotides in an RNA duplex, called bulges. The bulges may be naturally occurring or caused by diseases. When the SMSMs come into contact with the RNA transcript, it may be bound by the spliceosome or the other trans-factors, most notably RNA-binding proteins (RBPs). The SMSMs reported herein may cause an alteration in the sequence or abundance of the mature transcript, which may, in turn, cause a difference in the sequence or abundance of the functional protein should the transcript be protein-coding or the sequence or abundance of the functional RNA should the transcript be non-coding.

In some aspects, the present disclosure provides, inter alia, a compound of Formula (I):

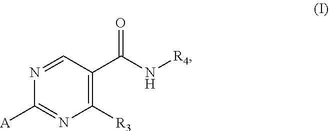

or a pharmaceutically acceptable salt, solvate, or prodrug thereof, wherein:

A is saturated or partially unsaturated mono-bi- or tri-cyclic 4- to 14-membered heterocycloalkyl or NR₁R₂, wherein the heterocycloalkyl comprises 1 or 2 nitrogen ring atoms and is optionally substituted with 1, 2, 3, or 4 R₆;
R₁ is —(CH₂)₀₋₂-heterocycloalkyl of 4-14 ring atoms comprising at least one nitrogen ring atom and 0-2 additional ring heteroatoms independently selected from N, O, and S; R₁ optionally substituted with 1, 2, 3, or 4 R₆;
R₂ is hydrogen, C₁₋₇alkyl, or C₃₋₈cycloalkyl;
R₃ is halo, C₁₋₇alkyl, OR₅, N(R₅)₂, C₃₋₈cycloalkyl, or heterocycloalkyl;
R₄ is aryl or bicyclic 9-membered heteroaryl comprising 2, 3, or 4 heteroatoms independently selected from N, O, and S, wherein R⁴ is optionally substituted with 1, 2, or 3 R₇;
each R₅ is independently C₁₋₇alkyl, C₁₋₇haloalkyl, C₃₋₈cycloalkyl, or heterocycloalkyl;
each R₆ is independently hydroxy, halogen, C₁₋₇alkyl, —(CH₂)₀₋₃—NRₐR_{b}, C₁₋₇heteroalkyl, —(CH₂)₀₋₃—C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen, OH, or C₁₋₇alkoxy and cycloalkyl and heterocycloalkyl are optionally substituted with 1-4 independently selected C₁₋₇alkyl, halogen, or OH;
or two R₆ together form oxo (═O);
or two R₆ together form C₁₋₇alkylene to form a ring;
each R′ is hydrogen or C₁₋₇alkyl;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
each Rₐ is independently H, C₁₋₇alkyl, C₁₋₇haloalkyl, or C₃₋₈cycloalkyl; and
each R_{b} is independently H, C₁₋₇alkyl, C₁₋₇haloalkyl, or C₃₋₈cycloalkyl.

In some aspects, the present disclosure provides a compound obtainable by, or obtained by, a method for preparing a compound as described here (e.g., a method comprising one or more steps described in herein).

In some aspects, the present disclosure provides a pharmaceutical composition comprising a compound of the present disclosure, or a pharmaceutically acceptable salt, solvate, or prodrug thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described herein).

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt, solvate, or prodrug thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt, solvate, or prodrug thereof, or a pharmaceutical composition of the present disclosure.

In some aspects, the present disclosure provides a compound of the present disclosure or a pharmaceutically acceptable salt, solvate, or prodrug thereof for use in treating or preventing a disease or disorder disclosed herein.

In some aspects, the present disclosure provides a compound of the present disclosure or a pharmaceutically acceptable salt, solvate, or prodrug thereof for use in treating a disease or disorder disclosed herein.

In some aspects, the present disclosure provides use of a compound of the present disclosure or a pharmaceutically acceptable salt, solvate, or prodrug thereof for treating or preventing a disease or disorder disclosed herein.

In some aspects, the present disclosure provides use of a compound of the present disclosure or a pharmaceutically acceptable salt, solvate, or prodrug thereof for treating a disease or disorder disclosed herein.

In some aspects, the present disclosure provides use of a compound of the present disclosure or a pharmaceutically acceptable salt, solvate, or prodrug thereof in the manufacture of a medicament for treating or preventing a disease or disorder disclosed herein.

In some aspects, the present disclosure provides use of a compound of the present disclosure or a pharmaceutically acceptable salt, solvate, or prodrug thereof in the manufacture of a medicament for treating a disease or disorder disclosed herein.

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the disclosure will be apparent from the following detailed description and claims.

### DETAILED DESCRIPTION

Compounds described herein are generally designed to treat diseases and disorders disclosed herein.

### Definitions

Unless otherwise stated, the following terms used in the specification and claims have the following meanings set out below.

As used herein, “alkyl”, “C₁, C₂, C₃, C₄, C₅, C₆, or C₇ alkyl” or “C₁-C₇ alkyl” is intended to include C₁, C₂, C₃, C₄, C₅, C₆, or C₇ straight chain (linear) saturated aliphatic hydrocarbon groups and C₃, C₄, C₅, C₆, or C₇ branched saturated aliphatic hydrocarbon groups. For example, C₁-C₇ alkyl is intends to include C₁, C₂, C₃, C₄, C₅, C₆ C₇ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₇ for straight chain, C₃-C₇ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₅). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” or “heterocyclyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1, l′-isobenzofuran]-yl, 7′H-spiro[cyclohexane-1,S′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” refers to a radical of a monocyclic or polycyclic (e.g., bicyclic or tricyclic)4n+2 aromatic ring system (e.g., having 6, 10, or 14× electrons shared in a cyclic array) having 6-14 ring carbon atoms and zero heteroatoms provided in the aromatic ring system. Examples of aryl groups include, but are not limited to, phenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom may be substituted or unsubstituted (i.e., N or NR wherein R is H or other substituents, as defined). The nitrogen and sulphur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “cyano” refers to the group-CN.

As used herein, the term “halo” or “halogen” refers to fluoro, chloro, bromo and iodo.

As used herein, the term “optionally substituted” refers to an unsubstituted group having designated substituents replacing one or more hydrogen atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocycloalkyl, alkylaryl, or an aromatic or heteroaromatic moiety.

As used herein, the term “haloalkyl” refers to an alkyl group having independently selected halogens replacing one or more hydrogen atoms. For example, C₁₋₇haloalkyl includes straight chain and branched hydrocarbons of 1 to 7 carbons with halogens replacing one or more hydrogens. Haloalkyl groups include but are not limited to CF₃, CH₂F, CH₂—CF₃, CHCICF₃, CF₂CF₃, CF(CH₃)>, CHBrCH₂F, and CH₂CHICH₂Br.

As used herein, the term “haloalkoxy” refers to alkoxy structures that are substituted with one or more halo groups or with combinations thereof. For example, the terms “fluoroalkyl” and “fluoro alkoxy” include haloalkyl and haloalkoxy groups, respectively, in which the halo is fluorine.

As used herein, the term “heteroalkyl” refers to straight chain and branched hydrocarbons having one or more carbons replaced by heteroatoms independently selected from N, O, and S. The hydrogens attached to said heteroatom are adjusted depending on the available valences of the replacement heteroatom. A CH₃ can be replaced with a NH₂, OH, or SH. A CH₂ can be replaced with a NH, O, or S. A CH can be replaced with N. For example, C₁₋₇heteroalkyl includes straight chain and branched hydrocarbons of 1 to 7 carbons having one or more carbons replaced by heteroatoms independently selected from N, O, and S. Thus, a C₇ heteroalkyl will have less than 7 carbons as at least one carbon will have been replaced with a heteroatom. Heteroalkyl groups include but are not limited to alkoxy groups such as methoxy, ethoxy, and isopropoxy; mono and dialkylamino groups such as methylamino, N,N-dimethylamino, and N-ethyl-N-methylamino, alkylthio groups such as methylthio and isopropylthio; and various other groups such as —CH₂OCH₃, —CH₂NH₂, —CH₂OCH₂CH₂OCH₃, —CH₂NHCH₂CH₃, —CH(OH)CH₂CH₃, —CH₂NHCH₂CH₂OCH₃, and —CH₂OCH(CH₃) CH₂SCH₃.

As used herein, “alkylene”, “C₁, C₂, C₃, C₄, C₅, C₆, or C₇ alkylene” or “C₁-C₇ alkylene” is a bivalent branched or straight alkyl group which has two open valences to connect the alkylene with two other groups. Examples of alkylene include —CH₂—, —CH₂CH₂—, —CH₂CH₂CH₂—, and —CH(CH₂CH₃)—CH₂—. Where an alkylene connects its open valences to the same atom or to separate atoms which are already part of a chain or ring, the alkylene taken together with the atom or atoms to which it is attached will form a ring.

As used herein, the term “alkoxy” refers to the group —OR where R is alkyl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e., with between 1 and 6 carbon atoms.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognised reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. It is to be understood that, unless otherwise stated, any description of a method of treatment includes use of the compounds to provide such treatment or prophylaxis as is described herein, as well as use of the compounds to prepare a medicament to treat such condition. The treatment includes treatment of human or non-human animals including rodents and other disease models.

As used herein, the term “subject” is interchangeable with the term “subject in need thereof”′, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a subject for the purpose of combating a disease, condition, or disorder and includes the administration of a compound of the present disclosure, or a pharmaceutically acceptable salt or solvate thereof, to alleviate the symptoms or complications of a disease, condition or disorder, or to eliminate the disease, condition or disorder. The term “treat” can also include treatment of a cell in vitro or an animal model.

It is to be appreciated that references to “treating” or “treatment” include the alleviation of established symptoms of a condition. “Treating” or “treatment” of a state, disorder or condition therefore includes: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N. Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the subject. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulphite, chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the subject should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition, and the therapeutic or combination of therapeutics selected for administration. Therapeutically effective amounts for a given situation can be determined by routine experimentation that is within the skill and judgment of the clinician.

A “therapeutically effective amount” means the amount of a compound that, when administered to a subject for treating a disease, is sufficient to effect such treatment for the disease. The “therapeutically effective amount” will vary depending on the compound, the disease and its severity and the age, weight, etc., of the subject to be treated.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred The dosage may vary within this range depending upon the dosage form employed, sensitivity of the subject, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASE, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient subject, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulphonic, acetic, ascorbic, benzene sulphonic, benzoic, bicarbonic, carbonic, citric, edetic, ethane disulphonic, 1,2-ethane sulphonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulphonic, maleic, malic, mandelic, methane sulphonic, napsylic, nitric, oxalic, pamoic, pantothenic, phenylacetic, phosphoric, polygalacturonic, propionic, salicylic, stearic, subacetic, succinic, sulphamic, sulphanilic, sulphuric, tannic, tartaric, toluene sulphonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulphonic acid, 2-naphthalenesulphonic acid, 4-toluenesulphonic acid, camphorsulphonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

A salt, for example, can be formed between an anion and a positively charged group (e.g., amino) on a substituted compound disclosed herein. Suitable anions include chloride, bromide, iodide, sulphate, bisulphate, sulphamate, nitrate, phosphate, citrate, methanesulphonate, trifluoroacetate, glutamate, glucuronate, glutarate, malate, maleate, succinate, fumarate, tartrate, tosylate, salicylate, lactate, naphthalenesulphonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulphonamides, tetrazoles, sulphonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate.

Compounds of any one of the Formulae disclosed herein may exist in a number of different tautomeric forms and references to compounds of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

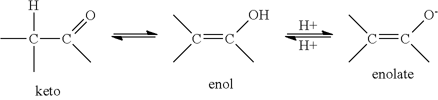

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substitutents at the ester or amide group in any one of the Formulae disclosed herein.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral centre” refers to a carbon atom bonded to four nonidentical substituents.

As used herein, the term “chiral isomer” means a compound with at least one chiral centre. Compounds with more than one chiral centre may exist either as an individual diastereomer or as a mixture of diastereomers, termed “diastereomeric mixture.” When one chiral centre is present, a stereoisomer may be characterised by the absolute configuration (R or S) of that chiral centre. Absolute configuration refers to the arrangement in space of the substituents attached to the chiral centre. The substituents attached to the chiral centre under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or(S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers).

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the subject without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8,1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the subject to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include «-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl,morpholinomethyl,piperazin-1-ylmethyl and 4-(C₁-C₄ alkyl) piperazin-1-ylmethyl.

The dosage regimen utilising the compounds is selected in accordance with a variety of factors including type, species, age, weight, sex and medical condition of the subject; the severity of the condition to be treated; the route of administration; the renal and hepatic function of the subject; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19th edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides, inter alia, a compound of Formula (I):

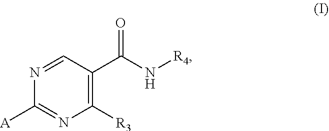

or a pharmaceutically acceptable salt, solvate, or prodrug thereof, wherein:
A is saturated or partially unsaturated mono-bi- or tri-cyclic 4- to 14-membered heterocycloalkyl or NR₁R₂, wherein the heterocycloalkyl comprises 1 or 2 nitrogen ring atoms and is optionally substituted with 1, 2, 3, or 4 R₆;
R₁ is —(CH₂)₀₋₂-heterocycloalkyl of 4-14 ring atoms comprising at least one nitrogen ring atom and 0-2 additional ring heteroatoms independently selected from N, O, and S; R₁ optionally substituted with 1, 2, 3, or 4 R₆;
R₂ is hydrogen, C₁₋₇alkyl, or C₃₋₇cycloalkyl;
R₃ is halo, C₁₋₇alkyl, OR₅, N(R₅)₂, C₃₋₈cycloalkyl, or heterocycloalkyl;
R₄ is aryl, or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₁₋₇haloalkyl, C₃₋₈cycloalkyl, or heterocycloalkyl;
each R₆ is independently hydroxy, halogen, C₁₋₇alkyl, (CH₂)₀₋₃—NRₐR_{b}, C₁₋₇heteroalkyl, —(CH₂)₀₋₃—C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen, OH, or C₁₋₇alkoxy, and cycloalkyl and heterocycloalkyl are optionally substituted with 1-4 independently selected C₁₋₇alkyl, halogen, or OH;
or two R₆ together form oxo (═O);
or two R₆ together form C₁₋₇alkylene to form a ring; each R′ is hydrogen or C₁₋₇alkyl;
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
each
Rₐ is independently H, C₁₋₇alkyl, C₁₋₇haloalkyl, or C₃₋₈cycloalkyl; and
each R_{b} is independently H, C₁₋₇alkyl, C₁₋₇haloalkyl, or C₃₋₈cycloalkyl.

In some aspects, the present disclosure provides a compound of Formula (I):

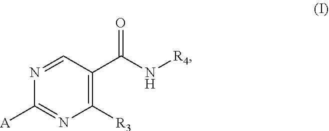

or a pharmaceutically acceptable salt, solvate, or prodrug thereof, wherein:

A is saturated or partially unsaturated mono- or bi-cyclic 4- to 11-membered nitrogen-containing heterocycloalkyl or NR₁R₂, wherein the nitrogen-containing heterocycloalkyl comprises 1 or 2 nitrogen ring atoms and is optionally substituted with 1, 2, 3, or 4 R₆,
R₁ is heterocycloalkyl comprising 1 nitrogen ring atom, optionally substituted with 1, 2, 3, or 4 R₆;
R₂ is hydrogen, C₁₋₇alkyl, or C₃₋₇cycloalkyl;
R₃ is C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ can be optionally substituted with 1, 2, or 3 R₇;
each R₅ is independently C₁₋₇alkyl, C₁₋₇haloalkyl, C₃₋₈cycloalkyl, or heterocycloalkyl;
each R₆ is independently hydroxy, halogen, C₁₋₇alkyl, —(CH₂)₀₋₃—NRₐR_{b}, C₁₋₇heteroalkyl, —(CH₂)₀₋₃—C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen, OH, or C₁₋₇alkoxy, and cycloalkyl and heterocycloalkyl are optionally substituted with 1-4 independently selected C₁₋₇alkyl, halogen, or OH;
or two R₅ together form oxo (═O);
or two R₆ together form C₁₋₇alkylene to form a ring; each R′ is hydrogen or C₁₋₇alkyl;
each R₇ is independently halo, C₁₋₇alkyl, C₁₋₇haloalkyl, C₁₋₇alkoxy, or C₁₋₇haloalkoxy;
each Rₐ is independently H, C₁₋₇alkyl, C₁₋₇haloalkyl, or C₃₋₈cycloalkyl; and
each R_{b} is independently H, C₁₋₇alkyl, C₁₋₇haloalkyl, or C₃₋₈cycloalkyl.

In some aspects, the present disclosure provides a compound of Formula (I):

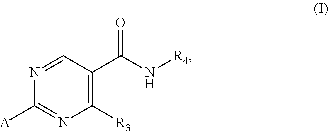

or a pharmaceutically acceptable salt, solvate, or prodrug thereof, wherein:

A is saturated or partially unsaturated mono-bi- or tri-cyclic 4- to 14-membered heterocycloalkyl or NR₁R₂, wherein the heterocycloalkyl comprises 1 or 2 nitrogen ring atoms and is optionally substituted with 1, 2, 3, or 4 R₆;
R₁ is —(CH₂)₀₋₂-heterocycloalkyl of 4-14 ring atoms comprising at least one nitrogen ring atom and 0-2 additional ring heteroatoms independently selected from N, O, and S; R₁ optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl, or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently hydroxy, C₁₋₇alkyl, amino, C₁₋₇heteroalkyl, —(CH₂)₀₋₃—C₃. &cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen or OH, and cycloalkyl and heterocycloalkyl are optionally substituted with 1-4 independently selected C₁₋₇alkyl, halogen, or OH;
or two R₆ together form oxo (═O);
or two R₆ together form C₁₋₇alkylene to form a ring;
each R′ is hydrogen or C₁₋₇alkyl; and
   each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH.

In some aspects, the present disclosure provides a compound of Formula (I):

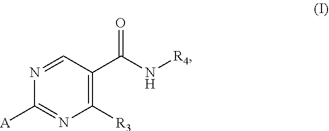

or a pharmaceutically acceptable salt, solvate, or prodrug thereof, wherein:

A is saturated or partially unsaturated mono-bi- or tri-cyclic 4- to 14-membered heterocycloalkyl or NR R₂, wherein the heterocycloalkyl comprises 1 or 2 nitrogen ring atoms and is optionally substituted with 1, 2, 3, or 4 R₆;
R₁ is —(CH₂)₀₋₂-heterocycloalkyl of 4-14 ring atoms comprising at least one nitrogen ring atom and 0-2 additional ring heteroatoms independently selected from N, O, and S; R₁ optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is bicyclic 9-membered heteroaryl comprising 2, 3, or 4 heteroatoms independently selected from N, O, and S, wherein Rₐ is optionally substituted with 1, 2, or 3 R₇;
each R₅ is independently C₁₋₇alkyl, C₃₋₈cycloalkyl, or heterocycloalkyl;
each R₆ is independently hydroxy, C₁₋₇alkyl, amino, C₁₋₇heteroalkyl, —(CH₂)₀₋₃-C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen or OH, and cycloalkyl and heterocycloalkyl are optionally substituted with 1-4 independently selected C₁₋₇alkyl, halogen, or OH;
or two R₆ together form oxo (═O);
or two R₆ together form C₁₋₇alkylene to form a ring;
each R′ is hydrogen or C₁₋₇alkyl; and
each R₇ is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH.

In some aspects, the present disclosure provides a compound of Formula (I):

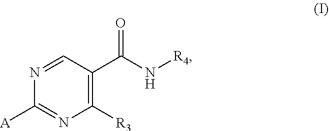

or a pharmaceutically acceptable salt, solvate, or prodrug thereof, wherein:

A is nitrogen-containing heterocycloalkyl or NR₁R₂, wherein the nitrogen-containing heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ is optionally substituted with 1, 2, or 3 R₇;
each R₅ is independently C₁₋₇alkyl, C₃₋₈cycloalkyl, or heterocycloalkyl;
each R₆ is independently C₁₋₇alkyl, amino, amino-C₁₋₇alkyl, C₃₋₈cycloalkyl, heterocycloalkyl, and C₁₋₇alkoxy-heterocycloalkyl, or two Re together form C₁₋₇alkylene; and
each R₇ is independently halo, C₁₋₇alkyl, C₁₋₇haloalkyl, C₁₋₇alkoxy, or C₁₋₇haloalkoxy.

It is understood that, for a compound of Formula (I), A, R₁, R₂, R₃, R₄, R₅, R₆, and R₇ can each be, where applicable, selected from the groups described herein, and any group described herein for any of A, R₁, R₂, R₃, R₄, R₅, R₆, and R₇ can be combined, where applicable, with any group described herein for one or more of the remainder of A, R₁, R₂. R₃, R₄, R₅, R₆, and R₇.

In some embodiments, the compound of formula (I) is of formula (Ia)

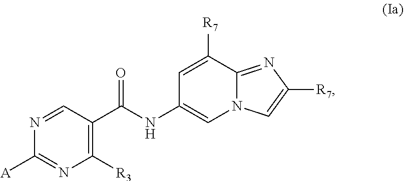

wherein variable definitions are as given herein.

In some embodiments, the compound of formula (I) is of formula (Ia)

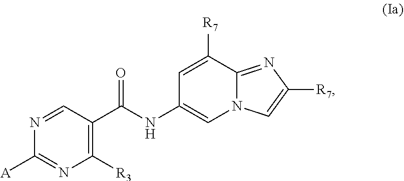

or a pharmaceutically acceptable salt, solvate, or prodrug thereof.

In some embodiments, A is saturated or partially unsaturated mono- or bi-cyclic 4- to 11-membered nitrogen-containing heterocycloalkyl, wherein A is attached through a nitrogen of the heterocycloalkyl.

In some embodiments, the compound of formula (I) is of formula (Ib)

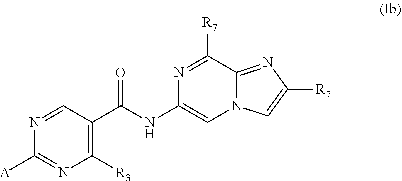

wherein variable definitions are as given herein.

In some embodiments, the compound of formula (I) is of formula (Ib)

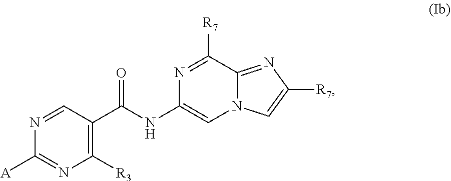

or a pharmaceutically acceptable salt, solvate, or prodrug thereof.

In some embodiments, A is saturated or partially unsaturated mono- or bi-cyclic 4- to 11-membered nitrogen-containing heterocycloalkyl, wherein A is attached through a nitrogen of the heterocycloalkyl.

In some embodiments, the compound of formula (I) is of formula (Ic)

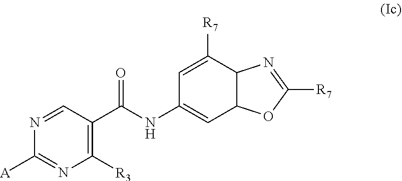

wherein variable definitions are as given herein.

In some embodiments, the compound of formula (I) is of formula (Ic)

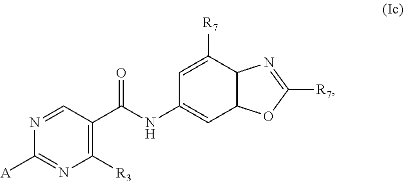

or a pharmaceutically acceptable salt, solvate, or prodrug thereof.

In some embodiments, A is saturated or partially unsaturated mono- or bi-cyclic 4- to 11-membered nitrogen-containing heterocycloalkyl, wherein A is attached through a nitrogen of the heterocycloalkyl.

In some embodiments, the compound of formula (I) is of formula (Id)

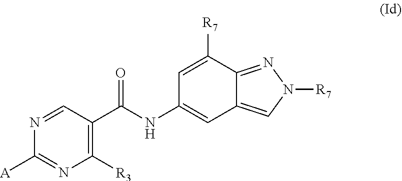

wherein variable definitions are as given herein.

In some embodiments, the compound of formula (I) is of formula (Id)

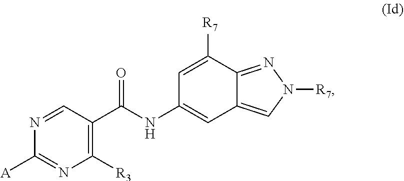

or a pharmaceutically acceptable salt, solvate, or prodrug thereof.

In some embodiments, A is saturated or partially unsaturated mono- or bi-cyclic 4- to 11-membered nitrogen-containing heterocycloalkyl, wherein A is attached through a nitrogen of the heterocycloalkyl.

In some embodiments,
A is 4-11 membered nitrogen-containing heterocycloalkyl, wherein the nitrogen-containing heterocycloalkyl comprises 1 or 2 nitrogen ring atoms and 0-2 additional ring heteroatoms selected from O and S, and is optionally substituted with 1, 2, 3, or 4 R₆;
each R₆ is independently C₁₋₇alkyl, —(CH₂)₀₋₃-NRₐR_{b}, C₃₋₈cycloalkyl, or heterocycloalkyl or two R₆ together form C₁₋₇alkylene to form a ring:

each Rₐ is independently H or C₁₋₇alkyl; and
each R_{b} is independently H or C₁₋₇alkyl.

In some embodiments, A is nitrogen-containing heterocycloalkyl, wherein the nitrogen-containing heterocycloalkyl comprises 1 or 2 nitrogen ring atoms and 0-2 additional ring heteroatoms selected from O and S, and is optionally substituted with 1, 2, 3, or 4 R₆, wherein each R₆ is independently C₁₋₇alkyl, amino, C₁₋₇alkylamino, C₃₋₈cycloalkyl, or heterocycloalkyl or two R₆ together form C₁₋₇alkylene.

In some embodiments, each R₆ is independently C₁₋₇ alkyl, or heterocycloalkyl, or two R₆ together form C₁₋₇alkylene. In some embodiments, each R₆ is independently methyl, ethyl, isopropyl, methoxy-azetidinyl, or pyrrolidinyl, or two R₆ together form ethylene or propylene.

In some embodiments, A is saturated or partially unsaturated mono- or bi-cyclic 4- to 9-membered heterocycloalkyl or NR₁R₂, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 9-membered heterocycloalkyl, wherein the heterocycloalkyl comprises 1 or 2 nitrogen ring atoms.

In some embodiments, A is saturated mono- or bi-cyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated mono- or bi-cyclic 4- to 9-membered heterocycloalkyl, wherein the heterocycloalkyl comprises 1 or 2 nitrogen ring atoms.

In some embodiments, A is partially unsaturated mono- or bi-cyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is partially unsaturated mono- or bi-cyclic 4- to 9-membered heterocycloalkyl, wherein the heterocycloalkyl comprises 1 or 2 nitrogen ring atoms.

In some embodiments, A is saturated monocyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated monocyclic 4- to 9-membered heterocycloalkyl, wherein the heterocycloalkyl comprises 1 or 2 nitrogen ring atoms.

In some embodiments, A is partially unsaturated monocyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is partially unsaturated monocyclic 4- to 9-membered heterocycloalkyl, wherein the heterocycloalkyl comprises 1 or 2 nitrogen ring atoms.

In some embodiments, A is saturated bicyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is saturated bicyclic 4- to 9-membered heterocycloalkyl, wherein the heterocycloalkyl comprises 1 or 2 nitrogen ring atoms.

In some embodiments, A is partially unsaturated bicyclic 4- to 9-membered heterocycloalkyl, wherein the heterocycloalkyl comprises 1 or 2 nitrogen ring atoms and is optionally substituted with 1, 2, 3, or 4 R₆.

In some embodiments, A is partially unsaturated bicyclic 4- to 9-membered heterocycloalkyl, wherein the heterocycloalkyl comprises 1 or 2 nitrogen ring atoms.

In some embodiments, A is saturated or partially unsaturated mono- or bicyclic 4- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 8-membered heterocycloalkyl, wherein the heterocycloalkyl comprises 1 or 2 nitrogen ring atoms.

In some embodiments, A is saturated or partially unsaturated mono- or bi-cyclic 4- to 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 7-membered heterocycloalkyl, wherein the heterocycloalkyl comprises 1 or 2 nitrogen ring atoms.

In some embodiments, A is saturated or partially unsaturated mono- or bi-cyclic 4- to 6-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 6-membered heterocycloalkyl, wherein the heterocycloalkyl comprises 1 or 2 nitrogen ring atoms.

In some embodiments, A is saturated or partially unsaturated mono- or bi-cyclic 4- to 5-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4- to 5-membered heterocycloalkyl, wherein the heterocycloalkyl comprises 1 or 2 nitrogen ring atoms.

In some embodiments, A is saturated or partially unsaturated mono- or bi-cyclic 5- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 5- to 9-membered heterocycloalkyl, wherein the heterocycloalkyl comprises 1 or 2 nitrogen ring atoms.

In some embodiments, A is saturated or partially unsaturated mono- or bi-cyclic 5- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 5- to 8-membered heterocycloalkyl, wherein the heterocycloalkyl comprises 1 or 2 nitrogen ring atoms.

In some embodiments, A is saturated or partially unsaturated mono- or bi-cyclic 5- to 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 5- to 7-membered heterocycloalkyl, wherein the heterocycloalkyl comprises 1 or 2 nitrogen ring atoms.

In some embodiments, A is saturated or partially unsaturated mono- or bi-cyclic 6- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6- to 9-membered heterocycloalkyl, wherein the heterocycloalkyl comprises 1 or 2 nitrogen ring atoms.

In some embodiments, A is saturated or partially unsaturated mono- or bi-cyclic 6- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6- to 8-membered heterocycloalkyl, wherein the heterocycloalkyl comprises 1 or 2 nitrogen ring atoms.

In some embodiments, A is saturated or partially unsaturated mono- or bi-cyclic 6- to 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6- to 7-membered heterocycloalkyl, wherein the heterocycloalkyl comprises 1 or 2 nitrogen ring atoms.

In some embodiments, A is saturated or partially unsaturated mono- or bi-cyclic 7- to 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 7- to 9-membered heterocycloalkyl, wherein the heterocycloalkyl comprises 1 or 2 nitrogen ring atoms.

In some embodiments, A is saturated or partially unsaturated mono- or bi-cyclic 7- to 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 7- to 8-membered heterocycloalkyl, wherein the heterocycloalkyl comprises 1 or 2 nitrogen ring atoms.

In some embodiments, A is saturated or partially unsaturated mono- or bi-cyclic 9-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 9-membered heterocycloalkyl, wherein the heterocycloalkyl comprises 1 or 2 nitrogen ring atoms.

In some embodiments, A is saturated or partially unsaturated mono- or bi-cyclic 8-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 8-membered heterocycloalkyl, wherein the heterocycloalkyl comprises 1 or 2 nitrogen ring atoms.

In some embodiments, A is saturated or partially unsaturated mono- or bi-cyclic 7-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 7-membered heterocycloalkyl, wherein the heterocycloalkyl comprises 1 or 2 nitrogen ring atoms.

In some embodiments, A is saturated or partially unsaturated mono- or bi-cyclic 6-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 6-membered heterocycloalkyl, wherein the heterocycloalkyl comprises 1 or 2 nitrogen ring atoms.

In some embodiments, A is saturated or partially unsaturated mono- or bi-cyclic 5-membered heterocycloalkyl, wherein the heterocycloalkyl comprises 1 or 2 nitrogen ring atoms and is optionally substituted with 1, 2, 3, or 4 Ró.

In some embodiments, A is saturated or partially unsaturated mono- or bi-cyclic 5-membered heterocycloalkyl, wherein the heterocycloalkyl comprises 1 or 2 nitrogen ring atoms.

In some embodiments, A is saturated or partially unsaturated mono- or bi-cyclic 4-membered heterocycloalkyl, wherein the heterocycloalkyl comprises 1 or 2 nitrogen ring atoms and is optionally substituted with 1, 2, 3, or 4 R₆.

In some embodiments, A is saturated or partially unsaturated mono- or bi-cyclic 4-membered heterocycloalkyl, wherein the heterocycloalkyl comprises 1 or 2 nitrogen ring atoms.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 1, 2, 3, or 4 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 1, 2, or 3 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 1 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 2 R₆.

In some embodiments, A is saturated or partially unsaturated mono- or bi-cyclic 4- to 9-membered heterocycloalkyl, wherein the heterocycloalkyl comprises 1 or 2 nitrogen ring atoms and is substituted with 3 R₆.

In some embodiments, A is saturated or partially unsaturated mono- or bi-cyclic 4- to 9-membered heterocycloalkyl, wherein the heterocycloalkyl comprises 1 nitrogen ring atom.

In some embodiments, A is saturated or partially unsaturated mono- or bi-cyclic 4- to 9-membered heterocycloalkyl, wherein the heterocycloalkyl comprises 2 nitrogen ring atoms.

In some embodiments, A is saturated or partially unsaturated mono- or bi-cyclic 4- to 9-membered heterocycloalkyl, wherein the heterocycloalkyl comprises 1 nitrogen ring atom and is optionally substituted with 1, 2, 3, or 4 R₆.

In some embodiments, A is saturated or partially unsaturated mono- or bi-cyclic 4- to 9-membered heterocycloalkyl, wherein the heterocycloalkyl comprises 2 nitrogen ring atoms and is optionally substituted with 1, 2, or 3 R₆.

In some embodiments, A is saturated or partially unsaturated mono- or bi-cyclic 4- to 9-membered heterocycloalkyl, wherein the heterocycloalkyl comprises 1 nitrogen ring atom and is substituted with 1, 2, 3, or 4 R₆.

In some embodiments, A is saturated or partially unsaturated mono- or bi-cyclic 4- to 9-membered heterocycloalkyl, wherein the heterocycloalkyl comprises 2 nitrogen ring atoms and is substituted with 1, 2, or 3 R₆.

In some embodiments, A is piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl, each optionally substituted with 1, 2, 3, or 4 R₆.

In some embodiments, A is

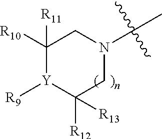

wherein,
Y is absent, N or CH;
R₉ is hydrogen, C₁₋₇alkyl, or (CH₂) m-NR₁₄R₁₅;
R₁₀ is hydrogen or Ct-? alkyl;
R₁₁ is hydrogen or C₁₋₇alkyl;
R₁₂ is hydrogen or C₁₋₇alkyl;
R₁₃ is hydrogen or C₁₋₇alkyl;
each R₁₄ and R₁₅ are independently hydrogen, C₁₋₇alkyl and C₃₋₈cycloalkyl;
n is 0, 1 or 2;
m is 0, 1 or 2;
or R₉ and R₁₀ together form C₁₋₇alkylene;
or R₉ and R₁₂ together form C₁₋₇alkylene;

or R₁₀ and R₁₁ together form C₂₋₇alkylene or 4- to 6-membered heterocycloalkyl optionally substituted with C₁-alkyl;

or R₁₀ and R₁₂ together form C₁₋₇alkylene or 4- to 6-membered heterocycloalkyl optionally substituted with C₁₋₇alkyl;

or R₁₀ and R₁₄ together form C₁₋₇alkylene;

or R₁₂ and R₁₃ together form C₂₋₇alkylene;

or R₁₂ and R₁₄ together form C₁₋₇alkylene;

or R₁₄ and R₁₅ together form C₂₋₇alkylene which is optionally substituted with C₁₋₇alkoxy.

In some embodiments, A is

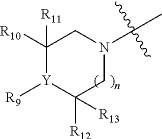

Y is absent, N or CH;
R₉ is hydrogen, C₁₋₇alkyl, or (CH₂)ₘ—NR₁₄R₁₅;
R₁₀ is hydrogen or C₁₋₇alkyl optionally substituted with one or more halo;
R₁₁ is hydrogen or C₁₋₇alkyl optionally substituted with one or more halo;
R₁₂ is hydrogen or C₁₋₇alkyl optionally substituted with one or more halo;
R₁₃ is hydrogen or C₁₋₇alkyl optionally substituted with one or more halo;
each R₁₄ and R₁₅ are independently hydrogen, C₁₋₇alkyl and C₃₋₈cycloalkyl;
n is 0, 1 or 2;
m is 0, 1 or 2;
or R₉ and R₁₀ together form C₁₋₇alkylene to form a ring;
or R₉ and R₁₅ together form C₁₋₇alkylene to form a ring;
or R₁₀ and R₁₁ together form C₂₋₇alkylene to form a ring or 4- to 6-membered heterocycloalkyl optionally substituted with C₁₋₇alkyl;
or R₁₀ and R₁₂ together form C₁₋₇alkylene to form a ring or 4- to 6-membered heterocycloalkyl optionally substituted with C₁₋₇alkyl;
or R₁₀ and R₁₄ together form C₁₋₇alkylene to form a ring;
or R₁₂ and R₁₅ together form C₂₋₇alkylene to form a ring;
or R₁₂ and R₁₄ together form C₁₋₇alkylene to form a ring;
or R₁₄ and R₁₅ together form C₂₋₇alkylene to form a ring.

In some embodiments, Y is N or CH. In some embodiments, Y is absent. In some embodiments, Y is N. In some embodiments, Y is CH.

In some embodiments, R₉ is hydrogen, C₁₋₇alkyl, or (CH₂)ₘ—NR₁₄R₁₅. In some embodiments, R₉ is hydrogen, pyrrolidinyl, or methoxy-azetidinyl. In some embodiments, R₉ is hydrogen. In some embodiments, Ry is C₁₋₇alkyl. In some embodiments, R₉ is (CH₂)ₘ—NR₁₄R₁₅.

In some embodiments, Y is CH and R₉ is (CH₂)ₘ—NR₁₄R₁₅.

In some embodiments, R₉ is —NH(CH₃), —N(CH₃)₂, —NH(CH₂CH₃), —CH₂NH(CH₃), or —CH₂N(CH₃)₂.

In some embodiments, R₁₀ is hydrogen or C₁₋₇alkyl. In some embodiments, R₁₀ is hydrogen, methyl, ethyl or isopropyl.

In some embodiments, R₁₀ is hydrogen. In some embodiments, R₁₀ is C₁₋₇alkyl.

In some embodiments, R₁₁ is hydrogen or C₁₋₇alkyl. In some embodiments, R₁₁ is hydrogen or methyl. In some embodiments, R₁₁ is hydrogen. In some embodiments, R₁₁ is C₁₋₇alkyl.

In some embodiments, R₁₂ is hydrogen or C₁₋₇alkyl. In some embodiments, R₁₁ is hydrogen or methyl. In some embodiments, R₁₂ is hydrogen. In some embodiments, R₁₂ is C₁₋₇alkyl.

In some embodiments, R₁₃ is hydrogen or C₁₋₇alkyl. In some embodiments, R₁₃ is hydrogen. In some embodiments, C₁₋₇alkyl.

In some embodiments, R 14 is hydrogen, C₁₋₇alkyl, or C₃₋₈cycloalkyl. In some embodiments, R₁₄ is hydrogen. In some embodiments, R: 4 is C₁₋₇alkyl. In some embodiments, R₁₄ is C₃₋₈cycloalkyl.

In some embodiments, R₁₅ is hydrogen, C₁₋₇alkyl, or C₃₋₈cycloalkyl. In some embodiments, R₁₅ is hydrogen. In some embodiments, R₁₅ is C₁₋₇alkyl. In some embodiments, R₁₅ is C₃₋₈cycloalkyl.

In some embodiments, n is 0, 1, or 2. In some embodiments, n is 0. In some embodiments, n is 1. In some embodiments, n is 2.

In some embodiments, m is 0, 1, or 2. In some embodiments, m is 0. In some embodiments, m is 1. In some embodiments, m is 2.

In some embodiments, R₉ and R₁₀ together form C₁₋₇alkylene. In some embodiments, Ry and Ria together form propylene to form a ring.

In some embodiments, R₉ and R₁₂ together form C₁₋₇alkylene.

In some embodiments, R₁₀ and R₁₁ together form C₂₋₇alkylene or 4- to 6-membered heterocycloalkyl optionally substituted with C₁₋₇alkyl. In some embodiments, R₁₀ and R₁₁ together form C₂₋₇alkylene. In some embodiments, R₁₀ and R₁₁ together form ethylene to form a ring.

In some embodiments, R₁₀ and R₁₁ together form 4- to 6-membered heterocycloalkyl optionally substituted with C₁₋₇alkyl. In some embodiments, R₁₀ and R₁₁ together form 4-membered heterocycloalkyl optionally substituted with C₁₋₇alkyl. In some embodiments, R₁₀ and R₁₁ together form 5-membered heterocycloalkyl optionally substituted with C₁₋₇alkyl. In some embodiments, R₁₀ and R₁₁ together form 6-membered heterocycloalkyl optionally substituted with C₁₋₇alkyl.

In some embodiments, R₁₀ and R₁₁ together form 4- to 6-membered heterocycloalkyl substituted with C₁₋₇alkyl. In some embodiments, R₁₀ and R₁₁ together form 4-membered heterocycloalkyl substituted with C₁₋₇alkyl. In some embodiments, R₁₀ and R₁₁ together form 5-membered heterocycloalkyl substituted with C₁₋₇alkyl. In some embodiments, R₁₀ and R₁₁ together form 6-membered heterocycloalkyl substituted with C₁₋₇alkyl.

In some embodiments, R₁₀ and R₁₁ together form C₁₋₇alkylene or 4- to 6-membered heterocycloalkyl optionally substituted with C₁₋₇alkyl. In some embodiments, R₁₀ and Rix together form C₁₋₇alkylene.

In some embodiments, R₁₀ and R₁₂ together form 4- to 6-membered heterocycloalkyl optionally substituted with C₁₋₇alkyl. In some embodiments, R₁₀ and R₁₂ together form 4-membered heterocycloalkyl optionally substituted with C₁₋₇alkyl. In some embodiments, R₁₀ and R₁₂ together form 5-membered heterocycloalkyl optionally substituted with C₁₋₇alkyl. In some embodiments, R₁₀ and R₁₂ together form 6-membered heterocycloalkyl optionally substituted with C₁₋₇alkyl.

In some embodiments, R₁₀ and R₁₂ together form 4- to 6-membered heterocycloalkyl substituted with C₁₋₇alkyl. In some embodiments, R₁₀ and R₁₂ together form 4-membered heterocycloalkyl substituted with C₁₋₇alkyl. In some embodiments, R₁₀ and R₁₂ together form 5-membered heterocycloalkyl substituted with C₁₋₇alkyl. In some embodiments, R₁₀ and R₁₂ together form 6-membered heterocycloalkyl substituted with C₁₋₇alkyl.

In some embodiments, R₁₀ and Ria together form C₁₋₇alkylene.

In some embodiments, R₁₂ and R₁₃ together form C₂₋₇alkylene.

In some embodiments, R₁₂ and R₁₄ together form C₁₋₇alkylene.

In some embodiments, R₁₄ and R₁₅ together form C₂₋₇alkylene. In some embodiments, R₁₄ and R₁₅ together form propylene or butylene to form a ring.

In some embodiments, R₁₄ and R₁₅ together form C₂₋₇alkylene which is optionally substituted with C₁₋₇alkoxy.

In some embodiments, R₁₄ and R₁₅ together form C₂₋₇alkylene which is substituted with C₁₋₇alkoxy.

In some embodiments, Y is CH and Ry is (CH₂)ₘ—NR₁₄R₁₅.

In some embodiments, Ry is hydrogen, pyrrolidinyl, or methoxy-azetidinyl.

In some embodiments, R₁₀ is hydrogen, methyl, ethyl or isopropyl.

In some embodiments, R₁₁ is hydrogen or methyl.

In some embodiments, R₁₂ is hydrogen or methyl.

In some embodiments, R₉ and R₁₀ together form propylene.

In some embodiments, R₁₀ and R₁₁ together form ethylene.

In some embodiments, R₁₄ and R₁₅ together form propylene or butylene.

In some embodiments, A is

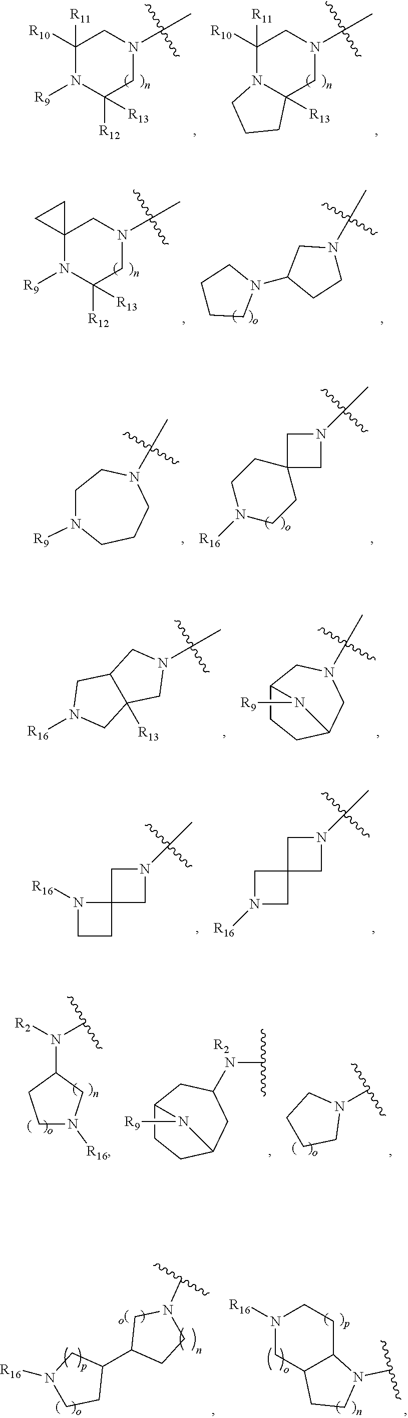

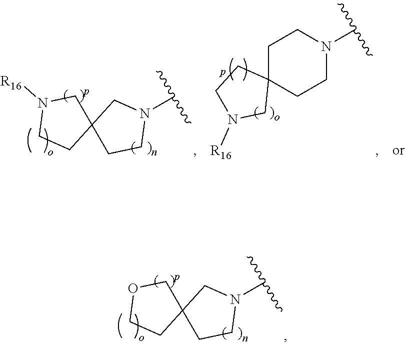

wherein R₉, R₁₀, R₁₁, R₁₂, and R₁₃ are as defined above; each R¹⁶ is independently hydrogen or C₁₋₇alkyl; each p is 0, 1, or 2; each o is 0, 1, or 2; and each A is optionally substituted with one or two R₆.

In some embodiments, A is

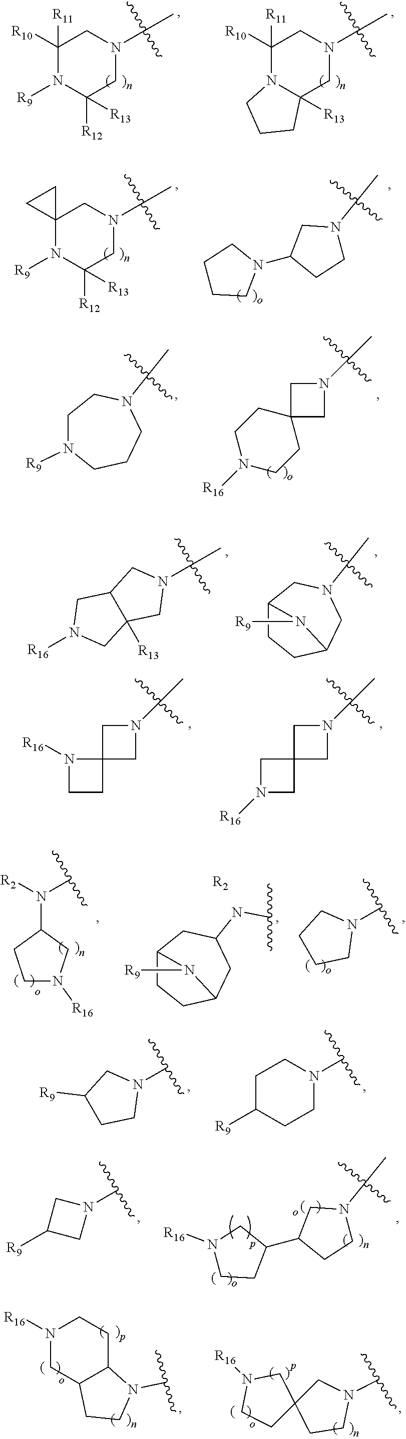

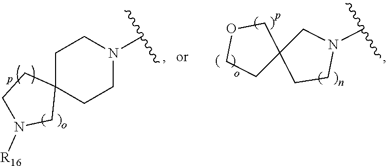

wherein R₉, R₁₀, R₁, R₁₂, and R₁₃ are as defined in any one of the preceding claims; each R¹⁶ is independently hydrogen or C₁₋₇alkyl; each p is 0, 1, or 2; each o is 0, 1, or 2; and each A is optionally substituted with one or two R₆.

In some embodiments, A is piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl, wherein piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl are each optionally substituted with 1, 2, 3, or 4 R₆.

In some embodiments, A is

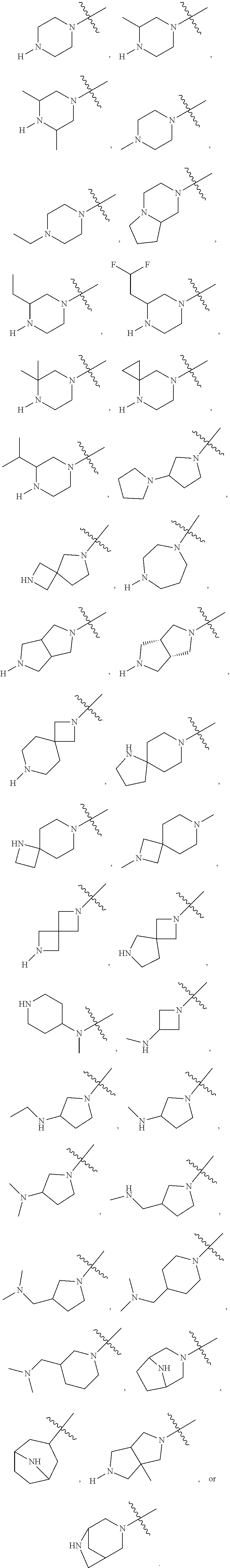

In some embodiments, A is

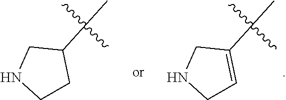 [SMILES: *C1CCNC1 ; *C1=CCNC1]

In some embodiments, R₁₆ is hydrogen or C₁₋₇alkyl. In some embodiments, R₁₆ is hydrogen. In some embodiments, R₁₆ is C₁₋₇alkyl.

In some embodiments A is

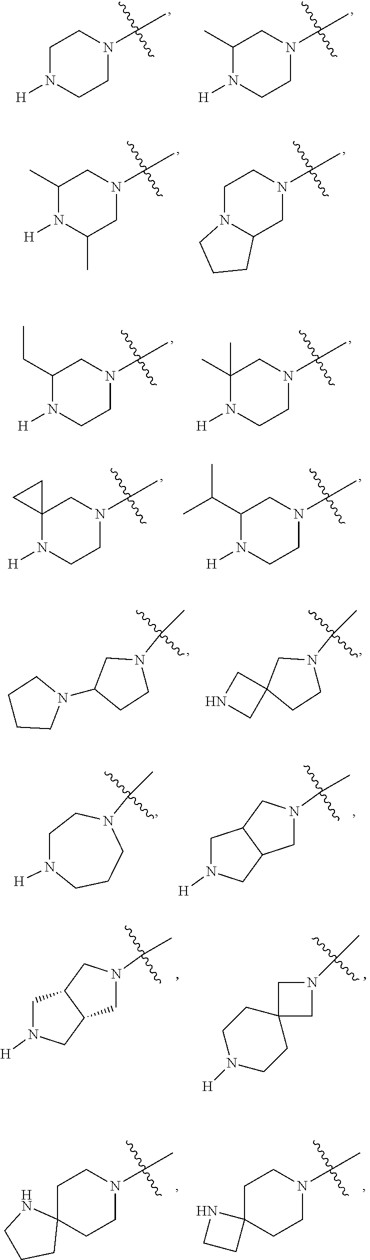

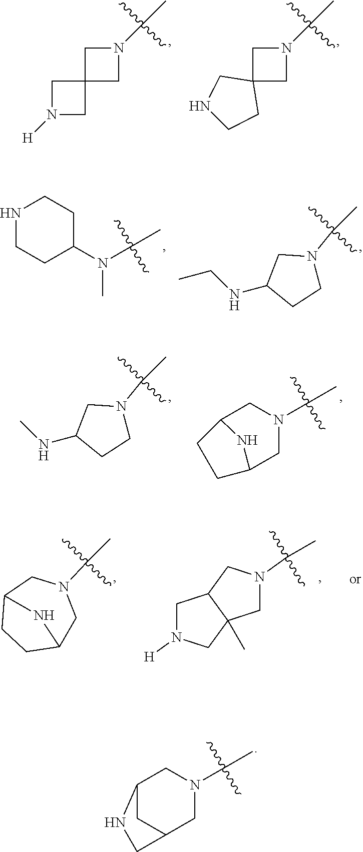

In some embodiments, A is NR₁R₂.

In some embodiments, R₁ is heterocycloalkyl comprising 1 nitrogen ring atom.

In some embodiments, R₁ is heterocycloalkyl comprising 1 nitrogen ring atom, wherein the heterocycloalkyl is optionally substituted with 1, 2, 3, or 4 R₆.

In some embodiments, R₁ is heterocycloalkyl comprising 1 nitrogen ring atom, wherein the heterocycloalkyl is substituted with 1, 2, 3, or 4 R₆.

In some embodiments, R₁ is heterocycloalkyl comprising 1 nitrogen ring atom, wherein the heterocycloalkyl is substituted with 1, 2, or 3 R₆.

In some embodiments, R₁ is heterocycloalkyl comprising 1 nitrogen ring atom, wherein the heterocycloalkyl is substituted with 1 R₆.

In some embodiments, R₁ is heterocycloalkyl comprising 1 nitrogen ring atom, wherein the heterocycloalkyl is substituted with 2 R₆.

In some embodiments, R₁ is heterocycloalkyl comprising 1 nitrogen ring atom, wherein the heterocycloalkyl is substituted with 3 R₆.

In some embodiments, R₁ is heterocycloalkyl comprising 1 nitrogen ring atom, wherein the heterocycloalkyl is substituted with 4 R₆.

In some embodiments, R₂ is hydrogen, C₁₋₇alkyl, or C₃₋₈cycloalkyl.

In some embodiments, R₂ is hydrogen or C₁₋₇alkyl.

In some embodiments, R₂ is hydrogen.

In some embodiments, R₂ is C₁₋₇alkyl. In some embodiments, R₂ is methyl. In some embodiments, R₂ is ethyl. In some embodiments, R₂ is propyl. In some embodiments, R₂ is butyl. In some embodiments, R₂ is pentyl. In some embodiments, R₂ is hexyl. In some embodiments, R₂ is heptyl. In some embodiments, R₂ is isopropyl. In some embodiments, R₂ is isobutyl. In some embodiments, R₂ is isopentyl. In some embodiments, R₂ is isohexyl. In some embodiments, R₂ is secbutyl. In some embodiments, R₂ is secpentyl. In some embodiments, R₂ is sechexyl. In some embodiments, R₂ is tertbutyl.

In some embodiments, R₂ is C₃₋₈cycloalkyl. In some embodiments, R₂ is cyclopropyl, cyclobutyl, cyclopentyl, cyclohexyl, cycloheptyl, or cyclooctyl.

In some embodiments, R₃ is H, halo, C₁₋₇alkyl, —OR₅, —N(R₅)₂, C₃₋₈cycloalkyl, or heterocycloalkyl.

In some embodiments, R₃ is H, C₁₋₇alkyl, —OR₅, —N(R₅)₂, C₃₋₈cycloalkyl, or heterocycloalkyl.

In some embodiments, R₃ is H or C₁₋₇alkyl.

In some embodiments, R₃ is H.

In some embodiments, R₃ is halo. In some embodiments, R₃ is F, Cl, Br, or I. In some embodiments, R₃ is F, Cl, or Br. In some embodiments, R₃ is F or C₁. In some embodiments, R₃ is F. In some embodiments, R₃ is C₁. In some embodiments. R₃ is Br. In some embodiments, R₅ is 1.

In some embodiments, R₃ is C₁₋₇alkyl. In some embodiments, R₃ is methyl. In some embodiments, R₃ is ethyl. In some embodiments, R₃ is propyl. In some embodiments, R₃ is butyl. In some embodiments, R₃ is pentyl. In some embodiments, R₃ is hexyl. In some embodiments, R₅ is heptyl. In some embodiments, R₃ is isopropyl. In some embodiments, R₃ is isobutyl. In some embodiments, R₃ is isopentyl. In some embodiments, R₃ is isohexyl. In some embodiments, R₃ is secbutyl. In some embodiments, R₃ is secpentyl. In some embodiments, R₅ is sechexyl. In some embodiments, R₃ is tertbutyl.

In some embodiments, R₃ is —OR₅ or —N(R₅)₂. In some embodiments, R₃ is —OR₅. In some embodiments, R₃ is —N(R₅)₂.

In some embodiments, R₃ is methoxy, ethoxy, or n-propoxy. In some embodiments, R₃ is methoxy. In some embodiments, R₃ is ethoxy.

In some embodiments, R₃ is C₃₋₈cycloalkyl or heterocycloalkyl.

In some embodiments, R₃ is C₃₋₈cycloalkyl. In some embodiments, R₃ is cyclopropyl, cyclobutyl, cyclopentyl, cyclohexyl, cycloheptyl, or cyclooctyl.

In some embodiments, R₃ is heterocycloalkyl.

In some embodiments, R₄ is aryl or bicyclic 9-membered heteroaryl comprising 2, 3, or 4 heteroatoms independently selected from N, O, or S.

In some embodiments, R₄ is aryl or bicyclic 9-membered heteroaryl comprising 2, 3, or 4 heteroatoms independently selected from N or O.

In some embodiments, R₄ is aryl.

In some embodiments, R₄ is aryl optionally substituted with one, two, or three R₇.

In some embodiments, Rₐ is aryl substituted with one, two, or three R₇. In some embodiments, R₄ is aryl substituted with one R₇. In some embodiments. R₄ is aryl substituted with two R₇. In some embodiments, Rₐ is aryl substituted with three R₇.

In some embodiments, R₄ is phenyl.

In some embodiments, R₄ is phenyl optionally substituted with one, two, or three R₇.

In some embodiments, Rₐ is phenyl substituted with one, two, or three R₇. In some embodiments, R₄ is phenyl substituted with one R₇. In some embodiments, R₄ is phenyl substituted with two R₇. In some embodiments, Rₐ is phenyl substituted with three R₇.

In some embodiments, R₄ is a bicyclic 9-membered heteroaryl comprising 2, 3, or 4 heteroatoms independently selected from N, O, and S.

In some embodiments, Rₐ is a bicyclic 9-membered heteroaryl comprising 2, 3, or 4 heteroatoms independently selected from N and O.

In some embodiments, R₄ is a bicyclic 9-membered heteroaryl comprising 2 or 3 heteroatoms independently selected from N, O, and S.

In some embodiments, Rₐ is a bicyclic 9-membered heteroaryl comprising 2 or 3 heteroatoms independently selected from N and O.

In some embodiments, R₄ is a bicyclic 9-membered heteroaryl comprising 2, 3, or 4 heteroatoms independently selected from N, O, and S, wherein R₄ is optionally substituted with 1, 2, or 3 R₇.

In some embodiments, R₄ is a bicyclic 9-membered heteroaryl comprising 2, 3, or 4 heteroatoms independently selected from N and O, wherein R₄ is optionally substituted 1, 2, or 3 R₇.

In some embodiments, R₄ is a bicyclic 9-membered heteroaryl comprising 2 or 3 heteroatoms independently selected from N. O, and S, wherein R₄ is optionally substituted with 1, 2, or 3 R₇.

In some embodiments, R₄ is a bicyclic 9-membered heteroaryl comprising 2 or 3 heteroatoms independently selected from N and O, wherein Rₐ is optionally substituted with 1, 2, or 3 R₇.

In some embodiments, Rₐ is a bicyclic 9-membered heteroaryl comprising 2, 3, or 4 heteroatoms independently selected from N and O, wherein R₄ is substituted with 1, 2, or 3 R₇.

In some embodiments, R₄ is a bicyclic 9-membered heteroaryl comprising 2 or 3 heteroatoms independently selected from N and O, wherein R₄ is substituted with 1, 2, or 3 R₇.

In some embodiments, R₄ is a bicyclic 9-membered heteroaryl comprising 2, 3, or 4 heteroatoms independently selected from N and O, wherein Rₐ is substituted with 1 R₇.

In some embodiments, R₄ is a bicyclic 9-membered heteroaryl comprising 2 or 3 heteroatoms independently selected from N and O, wherein R₄ is substituted with 1 R₇.

In some embodiments, Rₐ is a bicyclic 9-membered heteroaryl comprising 2, 3, or 4 heteroatoms independently selected from N and O, wherein Rₐ is substituted with 2 R₇.

In some embodiments, R₄ is a bicyclic 9-membered heteroaryl comprising 2 or 3 heteroatoms independently selected from N and O, wherein R₄ is substituted with 2 R₇.

In some embodiments, R is a bicyclic 9-membered heteroaryl comprising 2, 3, or 4 heteroatoms independently selected from N and O, wherein Rₐ is substituted with 3 R₇.

In some embodiments, R₄ is a bicyclic 9-membered heteroaryl comprising 2 or 3 heteroatoms independently selected from N and O, wherein Rₐ is substituted with 3 R₇.

In some embodiments, Rₐ is a bicyclic 9-membered heteroaryl comprising 2 heteroatoms independently selected from N and O, wherein R₄ is optionally substituted with 1, 2, or 3 R₇.

In some embodiments, Rₐ is a bicyclic 9-membered heteroaryl comprising two heteroatoms independently selected from N and O, wherein Rₐ is substituted with 1, 2, or 3 R₇.

In some embodiments, R₄ is a bicyclic 9-membered heteroaryl comprising 2 heteroatoms independently selected from N and O, wherein R₄ is substituted with 1 R₇.

In some embodiments, R₄ is a bicyclic 9-membered heteroaryl comprising 2 heteroatoms independently selected from N and O, wherein Rₐ is substituted with 2 R₇.

In some embodiments, R₄ is a bicyclic 9-membered heteroaryl comprising 2 heteroatoms independently selected from N and O, wherein R₄ is substituted with 3 R₇.

In some embodiments, R₄ is a bicyclic 9-membered heteroaryl comprising 3 heteroatoms independently selected from N and O, wherein Rₐ is optionally substituted with 1, 2, or 3 R₇.

In some embodiments, R is a bicyclic 9-membered heteroaryl comprising 3 heteroatoms independently selected from N and O, wherein R₄ is substituted with 1, 2, or 3 R₇.

In some embodiments, R₄ is a bicyclic 9-membered heteroaryl comprising 3 heteroatoms independently selected from N and O, wherein R₄ is substituted with 1 R₇.

In some embodiments, R₄ is a bicyclic 9-membered heteroaryl comprising 3 heteroatoms independently selected from N and O, wherein R₄ is substituted with 2 R₇.

In some embodiments, R₄ is a bicyclic 9-membered heteroaryl comprising 3 heteroatoms independently selected from N and O), wherein Rₐ is substituted with 3 R₇.

In some embodiments, R₄ is a bicyclic 9-membered heteroaryl comprising four heteroatoms independently selected from N and O, wherein R₄ is optionally substituted with 1, 2, or 3 R₇.

In some embodiments, R₄ is a bicyclic 9-membered heteroaryl comprising 4 heteroatoms independently selected from N and O, wherein R₄ is substituted with 1, 2, or 3 R₇.

In some embodiments, R₄ is a bicyclic 9-membered heteroaryl comprising 4 heteroatoms independently selected from N and O, wherein Rₐ is substituted with 1 R₇.

In some embodiments, R₄ is a bicyclic 9-membered heteroaryl comprising 4 heteroatoms independently selected from N and O, wherein R₄ is substituted with 2 R₇.

In some embodiments, R₄ is a bicyclic 9-membered heteroaryl comprising 4 heteroatoms independently selected from N and O, wherein Rₐ is substituted with 3 R₇.

In some embodiments, R₄ is selected from imidazo[1,2-a]pyrazine, benzo[d]oxazole, or imidazo[1,2-a]pyrazine each optionally substituted with 1, 2, 3 or 4 R₇.

In some embodiments, R₄ is a bicyclic 9-membered heteroaryl comprising 2 heteroatoms independently selected from N and O, wherein Rₐ can be optionally substituted with 1, 2, or 3 R₇.

In some embodiments, R₄ is a bicyclic 9-membered heteroaryl comprising 2 heteroatoms independently selected from N and O, wherein R₄ is substituted with 1 or 2 R₇.

In some embodiments, R₄ is

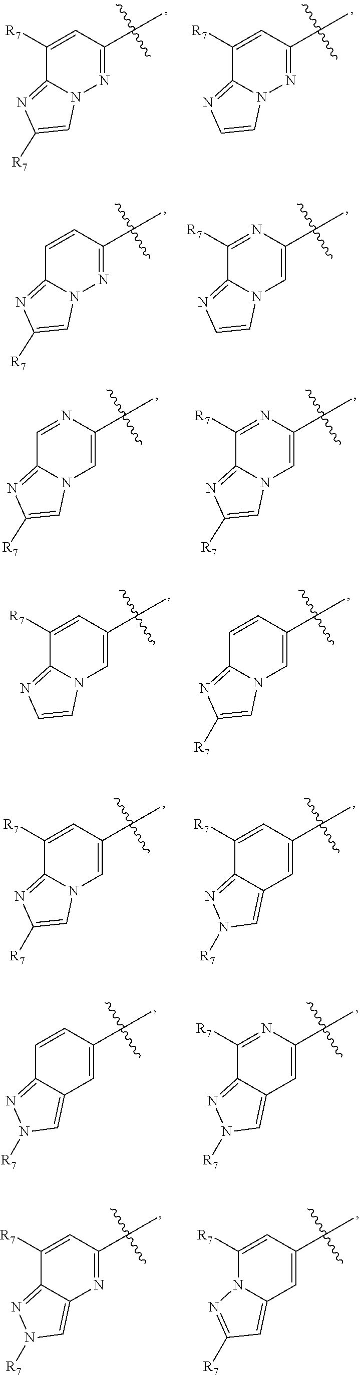

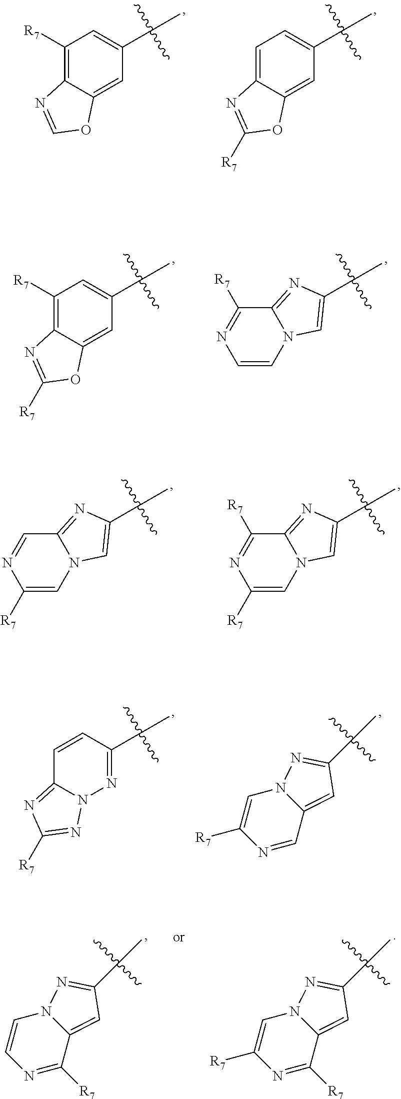

In some embodiments. Rₐ is

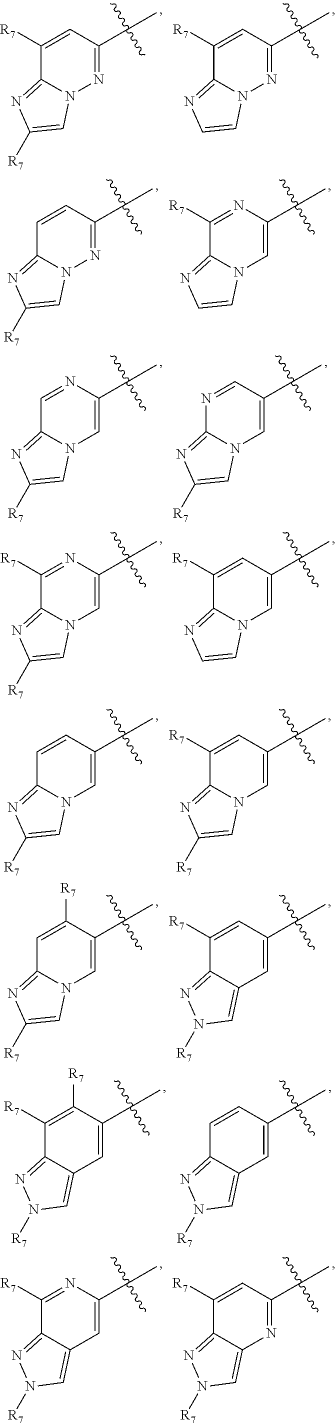

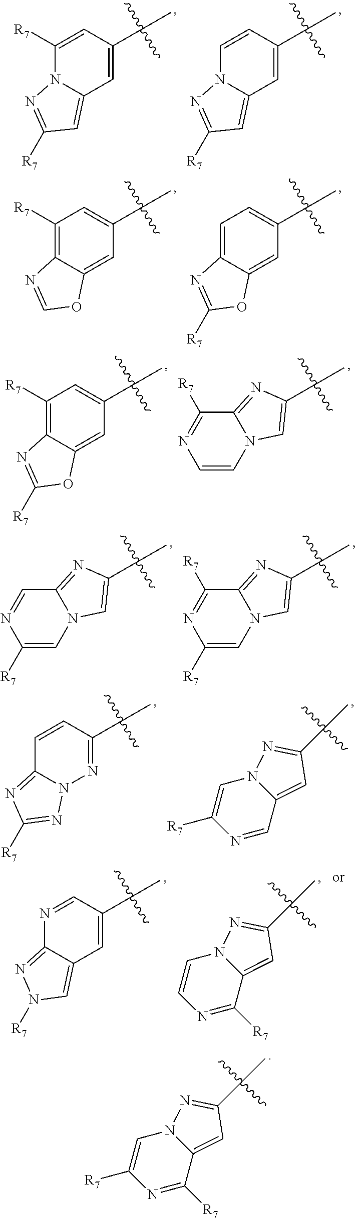

In some embodiments, R is

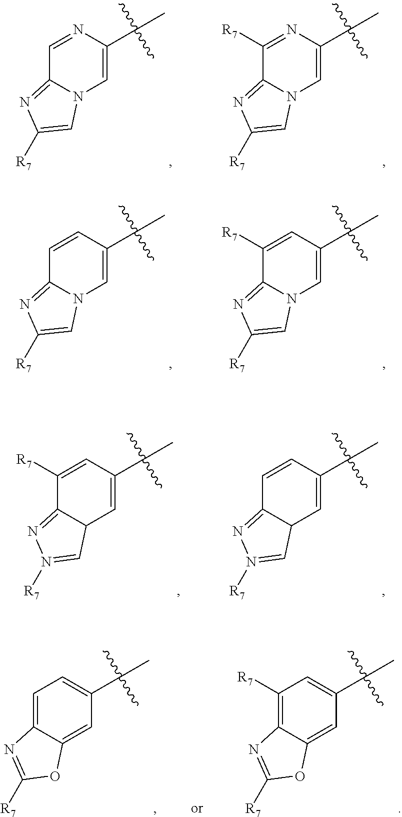

In some embodiments, Rₐ is selected from imidazo[1,2-a]pyrazine, benzo[d]oxazole, or imidazo[1,2-a]pyrazine, wherein imidazo[1,2-a]pyrazine, benzo[d]oxazole, or imidazo[1,2-a]pyrazine are each optionally substituted with 1, 2, 3 or 4 R₇.

In some embodiments, Rₐ is

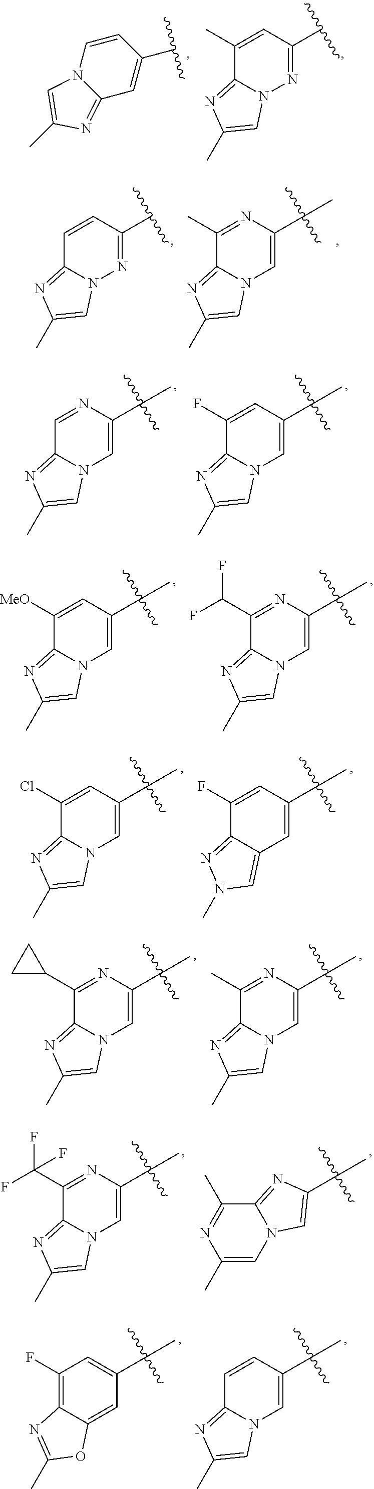

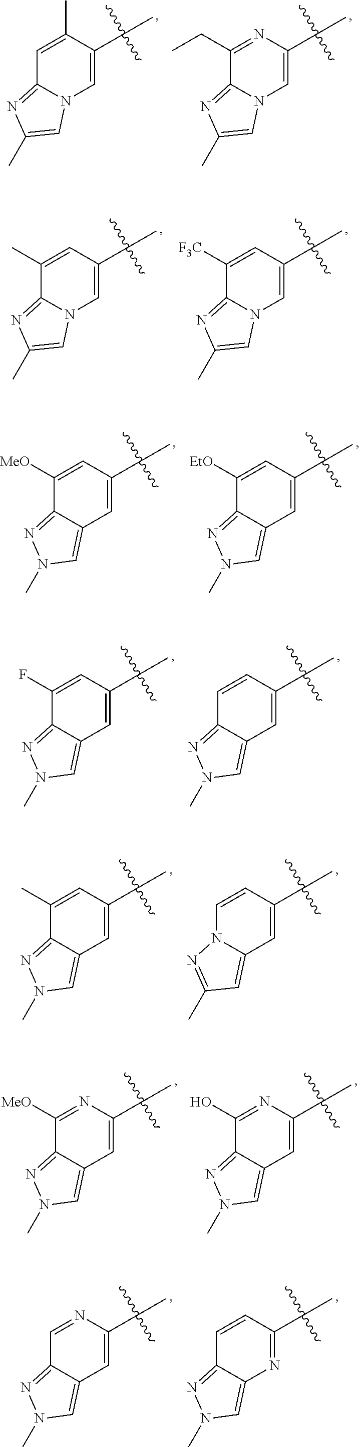

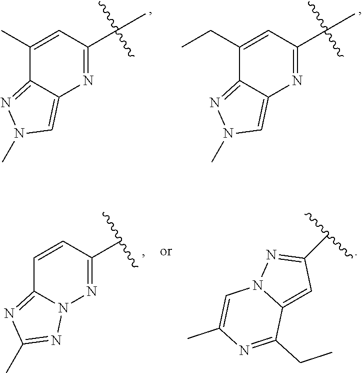 [SMILES: *c1cc(C)c2nn(C)cc2n1 ; *c1cc(CC)c2nn(C)cc2n1 ; *c1ccc2nc(C)nn2n1 ; *c1cc2c(CC)nc(C)cn2n1]

In some embodiments. R₄ is

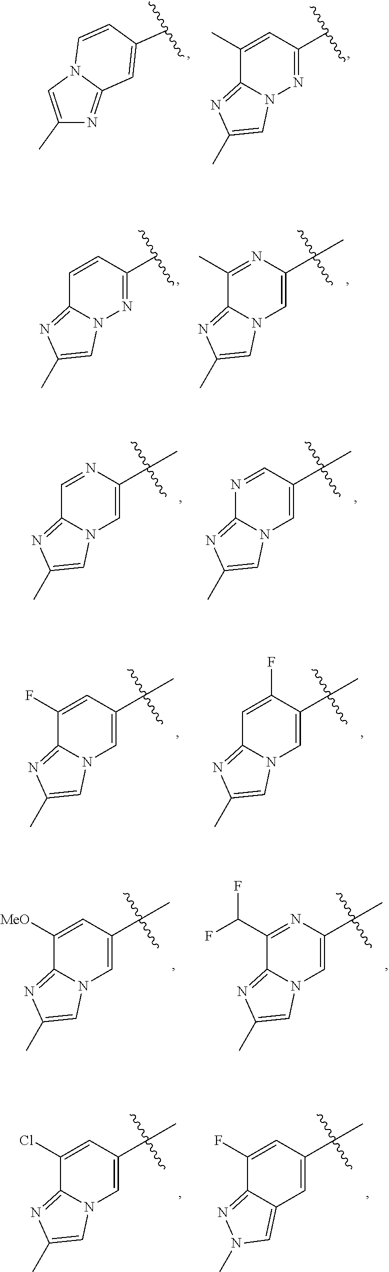

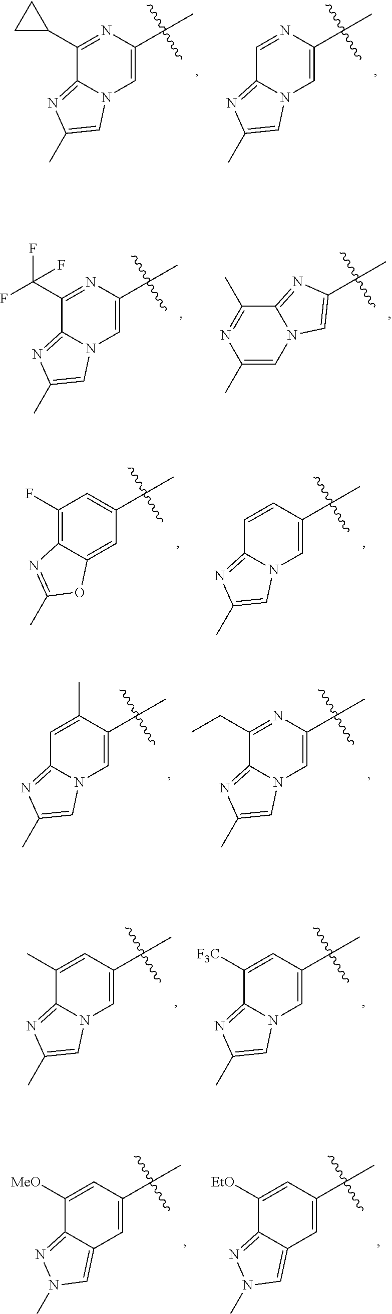

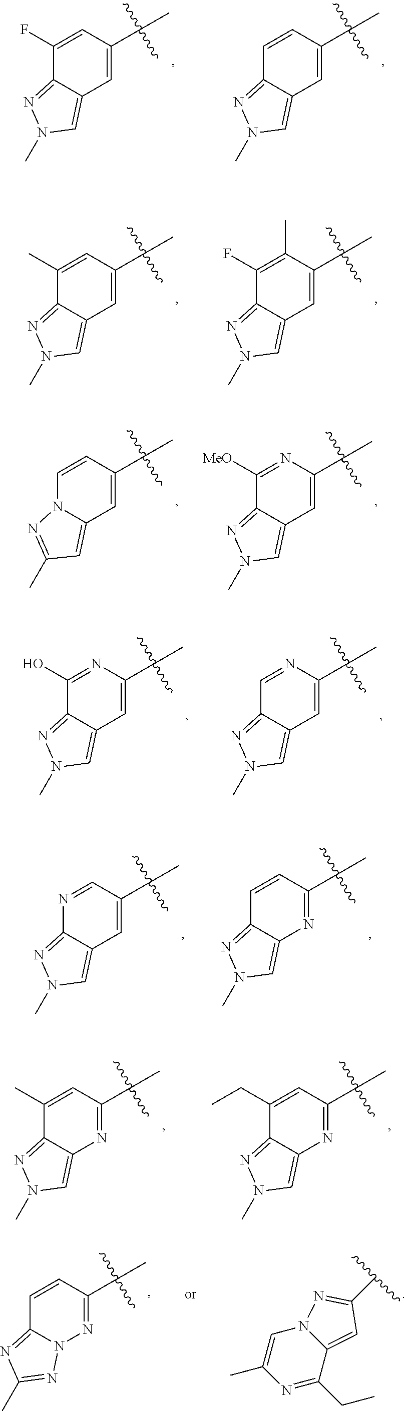

In some embodiments, R₄ is

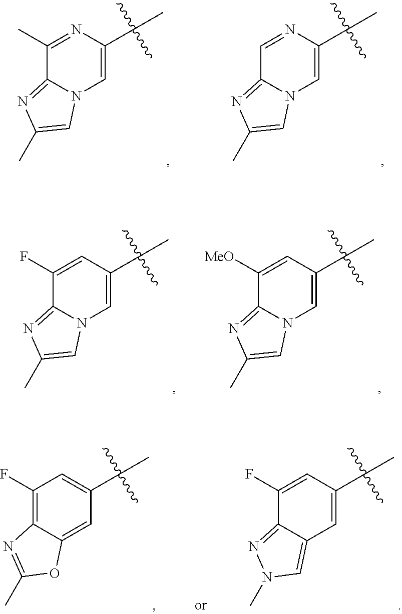 [SMILES: *c1cn2cc(C)nc2c(C)n1 ; *c1cn2cc(C)nc2cn1 ; *c1cc(F)c2nc(C)cn2c1 ; *c1cc(OC)c2nc(C)cn2c1 ; *c1cc(F)c2nc(C)oc2c1 ; *c1cc(F)c2nn(C)cc2c1]

In some embodiments, each R₅ is independently C₁₋₇alkyl, C₃₋₈cycloalkyl, or heterocycloalkyl.

In some embodiments, each R₅ is independently C₁₋₇alkyl. In some embodiments, each R₅ is independently methyl. In some embodiments, each R₅ is independently ethyl. In some embodiments, each R₅ is independently propyl. In some embodiments, each R₅ is independently butyl. In some embodiments, each R₅ is independently pentyl. In some embodiments, each R₅ is independently hexyl. In some embodiments, each R₅ is independently heptyl. In some embodiments, each R₅ is independently isopropyl. In some embodiments, each R₅ is independently isobutyl. In some embodiments, each R₅ is independently isopentyl. In some embodiments, each R₅ is independently isohexyl. In some embodiments, each R₅ is independently secbutyl. In some embodiments, each R₅ is independently secpentyl. In some embodiments, each R₅ is independently sechexyl. In some embodiments, each R₅ is independently tertbutyl.

In some embodiments, each R₅ is independently C₃₋₈cycloalkyl or heterocycloalkyl.

In some embodiments, each R₅ is independently C₃₋₈cycloalkyl. In some embodiments, R₅ is cyclopropyl, cyclobutyl, cyclopentyl, cyclohexyl, cycloheptyl, or cyclooctyl.

In some embodiments, each R₅ is independently heterocycloalkyl.

In some embodiments, each R₆ is independently C₁₋₇alkyl, amino, C₁₋₇alkylamino, C₃₋₈cycloalkyl, or heterocycloalkyl or two Re together form C₁₋₇alkylene.

In some embodiments, each R₆ is independently C₁₋₇ alkyl or heterocycloalkyl or two R₆ together form C₁₋₇alkylene to form a ring. In some embodiments, each R₆ is independently methyl, ethyl, isopropyl, methoxy-azetidinyl, or pyrrolidinyl, or two R₆ together form ethylene or propylene to form a ring.

In some embodiments, each R₆ is independently —(CH₂)₀₋₃—NRₐR_{b}.

In some embodiments, Rₐ is H and Re is C₁₋₇alkyl. In some embodiments, Rₐ and R_{b} are each independently C₁₋₇alkyl. In some embodiments, Rₐ and R_{b} are each independently H.

In some embodiments, each R₆ is independently —NH(CH₃), —N(CH₃)₂, —NH(CH₂CH₃), —CH₂NH(CH₃), or —CH₂N(CH₃)₂. In some embodiments, each R₆ is independently —NH(CH₃). In some embodiments, each R₆ is —N(CH₃)₂. In some embodiments, each R₆ is independently-NH(CH₂CH₃). In some embodiments, each R₆ is independently-CH₂NH(CH₃). In some embodiments, each R₆ is independently-CH₂N(CH₃)₂.

In some embodiments, each R₆ is independently C₁₋₇alkyl, amino, C₁₋₇alkylamino, C₃₋₈cycloalkyl, or heterocycloalkyl.

In some embodiments, each R₆ is independently C₁₋₇alkyl. In some embodiments, each R₆ is independently methyl. In some embodiments, each R₆ is independently ethyl. In some embodiments, each R₆ is independently propyl. In some embodiments, each R₆ is independently butyl. In some embodiments, each R₆ is independently pentyl. In some embodiments, each R₆ is independently hexyl. In some embodiments, each R₆ is independently heptyl. In some embodiments, each R₆ is independently isopropyl. In some embodiments, each R₆ is independently isobutyl. In some embodiments, each R₆ is independently isopentyl. In some embodiments, each R₆ is independently isohexyl. In some embodiments, each R₆ is independently secbutyl. In some embodiments, each R₆ is independently secpentyl. In some embodiments, each R₆ is independently sechexyl. In some embodiments, each R₆ is independently tertbutyl.

In some embodiments, each R₆ is independently amino or amino-C₁₋₇alkyl.

In some embodiments, each R₆ is independently amino. In some embodiments, R₆ is independently amino-C₁₋₇alkyl.

In some embodiments, each R₆ is independently C₃₋₈cycloalkyl, heterocycloalkyl and C₁₋₇alkoxy-heterocycloalkyl.

In some embodiments, each R₆ is independently C₃₋₈cycloalkyl.

In some embodiments, each R₆ is independently heterocycloalkyl and C₁₋₇alkoxy-heterocycloalkyl.

In some embodiments, each R₆ is independently heterocycloalkyl. In some embodiments, each R₆ is independently C₁₋₇alkoxy-heterocycloalkyl.

In some embodiments, two R₆ together form C₁₋₇alkylene.

In some embodiments, each R₇ is independently halo, cyano, C₁₋₇alkyl, C₁₋₇haloalkyl, C₁₋₇alkoxy C₁₋₇haloalkoxy, or C₃₋₈cycloalkyl.

In some embodiments, each R₇ is independently halo or cyano.

In some embodiments, each R; is independently halo. In some embodiments, each R₇ is independently from F, Cl, Br, and I. In some embodiments, each R₇ is independently from F, Cl, or Br. In some embodiments, each Ry is independently F. In some embodiments, each R₇ is independently C₁. In some embodiments, each R₇ is independently Br. In some embodiments, each R₇ is independently I.

In some embodiments, each R₇ is independently cyano.

In some embodiments, each R₇ is independently from C₁₋₇alkyl, C₁₋₇haloalkyl, C₁₋₇alkoxy C₁₋₇haloalkoxy, or C₃₋₈cycloalkyl.

In some embodiments, each R₇ is independently from C₁₋₇alkyl, C₁₋₇haloalkyl, C₁₋₇alkoxy, or C₁₋₇haloalkoxy.

In some embodiments, each R₇ is independently C₁₋₇alkyl. In some embodiments, each R₇ is independently methyl. In some embodiments, each R₇ is independently ethyl. In some embodiments, each R₇ is independently propyl. In some embodiments, each R₇ is independently butyl. In some embodiments, each R₇ is independently pentyl. In some embodiments, each R₇ is independently hexyl. In some embodiments, each R₇ is independently heptyl. In some embodiments, each R₇ is independently isopropyl In some embodiments, each R₇ is independently isobutyl. In some embodiments, each R₇ is independently isopentyl. In some embodiments, each R₇ is independently isohexyl. In some embodiments, each R₇ is independently secbutyl. In some embodiments, each R₇ is independently secpentyl. In some embodiments, each R₇ is independently sechexyl. In some embodiments, each R₇ is independently tertbutyl.

In some embodiments, each R₇ is independently C₁₋₇alkyl optionally substituted with OH.

In some embodiments, each R₇ is independently C₁₋₇alkyl substituted with OH. In some embodiments, each R₇ is independently methyl substituted with OH. In some embodiments, each R₇ is independently ethyl substituted with OH. In some embodiments, each R₇ is independently propyl substituted with OH. In some embodiments, each R₇ is independently butyl substituted with OH. In some embodiments, each R₇ is independently pentyl substituted with OH. In some embodiments, each R₇ is independently hexyl substituted with OH. In some embodiments, each R₇ is independently heptyl substituted with OH. In some embodiments, each R₇ is independently isopropyl substituted with OH. In some embodiments, each R₇ is independently isobutyl substituted with OH. In some embodiments, each R₇ is independently isopentyl substituted with OH. In some embodiments, each R₇ is independently isohexyl substituted with OH. In some embodiments, each R is independently secbutyl substituted with OH. In some embodiments, each R₇ is independently secpentyl substituted with OH. In some embodiments, each R₇ is independently sechexyl substituted with OH. In some embodiments, each R₇ is independently tertbutyl substituted with OH.

In some embodiments, each R; is independently C₁₋₇haloalkyl, C₁₋₇alkoxy, or C₁₋₇haloalkoxy.

In some embodiments, each R₇ is independently Ct-7haloalkyl.

In some embodiments, each R₇ is independently C₁₋₇alkoxy.

In some embodiments, each R₇ is independently C₁₋₇haloalkoxy.

In some embodiments, each R₇ is independently C₃₋₈cycloalkyl.

In some embodiments, the compound is selected from the compounds described in Table 1 and pharmaceutically acceptable salts, solvates, or prodrugs thereof.

In some embodiments, the compound is selected from the compounds described in Table 1 and pharmaceutically acceptable salts thereof.

In some embodiments, the compound is selected from the prodrugs of compounds described in Table 1 and pharmaceutically acceptable salts thereof.

In some embodiments, the compound is selected from the compounds described in Table 1.

TABLE 1

| Compound No. | Structure |
|---|---|
| 1 | 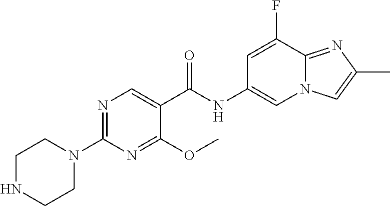 [SMILES: COc1nc(N2CCNCC2)ncc1C(=O)Nc1cc(F)c2nc(C)cn2c1] |
| 2 | 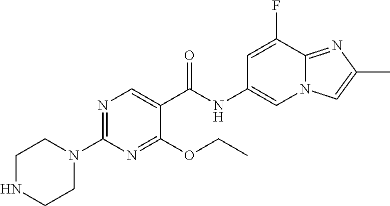 [SMILES: CCOc1nc(N2CCNCC2)ncc1C(=O)Nc1cc(F)c2nc(C)cn2c1] |
| 3 | 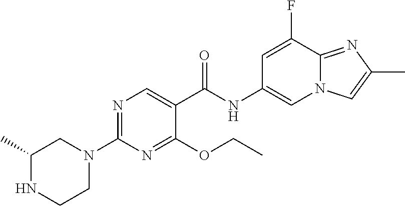 |
| 4 | 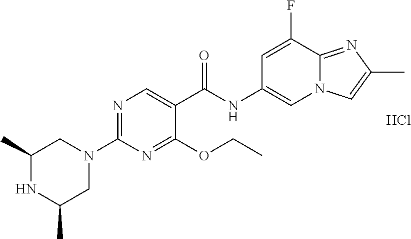 |
| 5 | 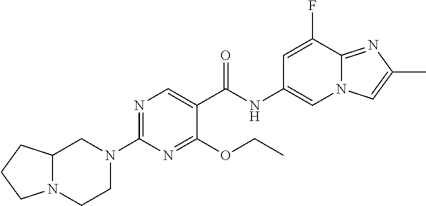 [SMILES: CCOc1nc(N2CCN3CCCC3C2)ncc1C(=O)Nc1cc(F)c2nc(C)cn2c1] |
| 6 | 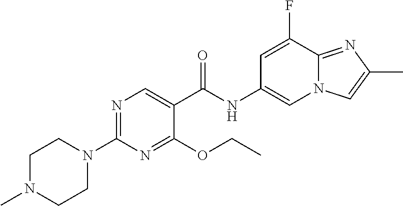 |
| 7 | 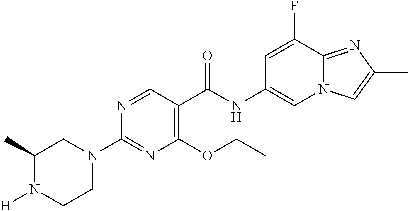 |
| 8 | 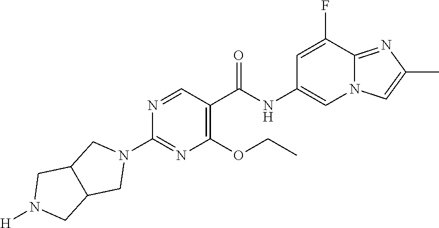 [SMILES: CCOc1nc(N2CC3CNCC3C2)ncc1C(=O)Nc1cc(F)c2nc(C)cn2c1] |
| 9 | 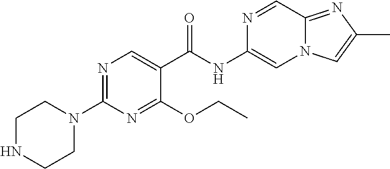 [SMILES: CCOc1nc(N2CCNCC2)ncc1C(=O)Nc1cn2cc(C)nc2cn1] |
| 10 | 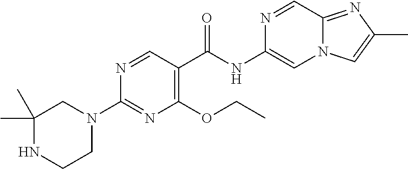 |
| 11 | 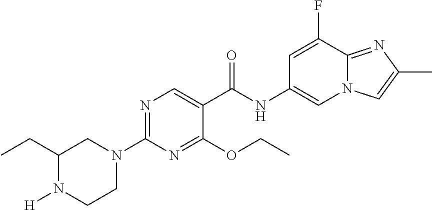 [SMILES: CCOc1nc(N2CCNC(CC)C2)ncc1C(=O)Nc1cc(F)c2nc(C)cn2c1] |
| 12 | 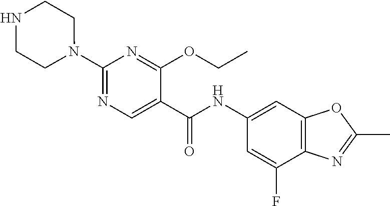 [SMILES: CCOc1nc(N2CCNCC2)ncc1C(=O)Nc1cc(F)c2nc(C)oc2c1] |
| 13 | 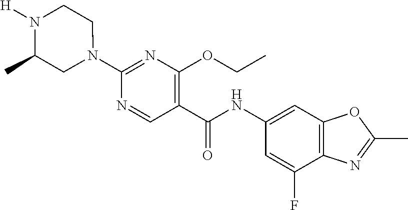 [SMILES: CCOc1nc(N2CCN[C@H](C)C2)ncc1C(=O)Nc1cc(F)c2nc(C)oc2c1] |
| 14 | 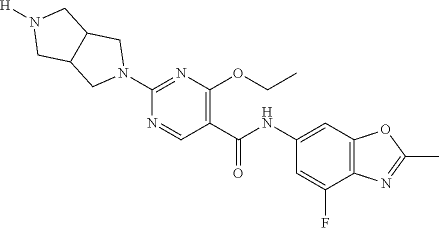 [SMILES: CCOc1nc(N2CC3CNCC3C2)ncc1C(=O)Nc1cc(F)c2nc(C)oc2c1] |
| 15 | 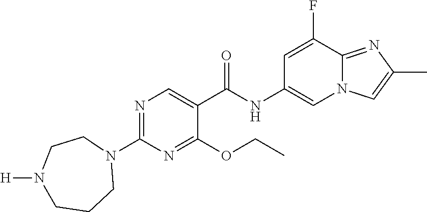 [SMILES: CCOc1nc(N2CCCNCC2)ncc1C(=O)Nc1cc(F)c2nc(C)cn2c1] |
| 16 | 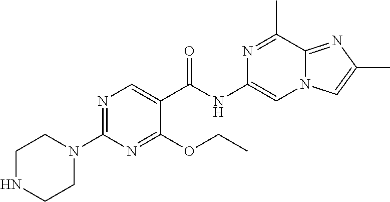 [SMILES: CCOc1nc(N2CCNCC2)ncc1C(=O)Nc1cn2cc(C)nc2c(C)n1] |
| 17 | 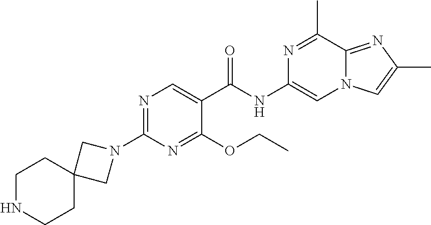 [SMILES: CCOc1nc(N2CC3(CCNCC3)C2)ncc1C(=O)Nc1cn2cc(C)nc2c(C)n1] |
| 18 | 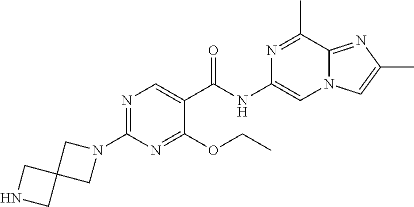 [SMILES: CCOc1nc(N2CC3(CNC3)C2)ncc1C(=O)Nc1cn2cc(C)nc2c(C)n1] |
| 19 | 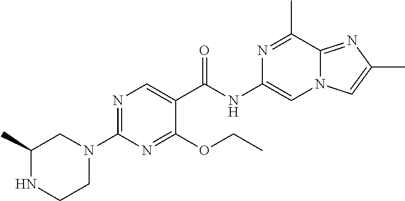 |
| 20 | 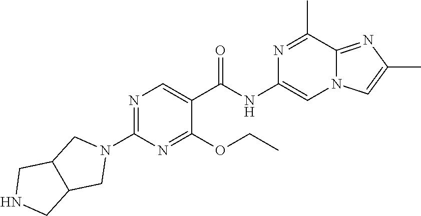 [SMILES: CCOc1nc(N2CC3CNCC3C2)ncc1C(=O)Nc1cn2cc(C)nc2c(C)n1] |
| 21 | 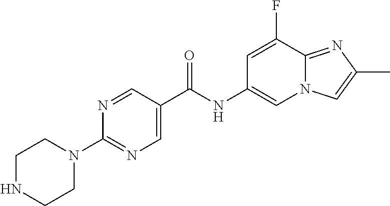 [SMILES: Cc1cn2cc(NC(=O)c3cnc(N4CCNCC4)nc3)cc(F)c2n1] |
| 22 | 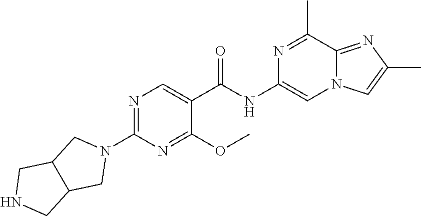 [SMILES: COc1nc(N2CC3CNCC3C2)ncc1C(=O)Nc1cn2cc(C)nc2c(C)n1] |
| 23 | 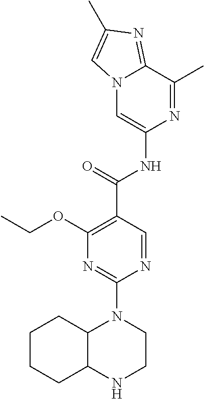 [SMILES: CCOc1nc(N2CCNC3CCCCC32)ncc1C(=O)Nc1cn2cc(C)nc2c(C)n1] |
| 24 | 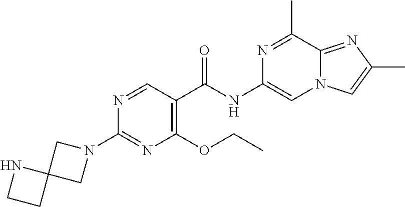 [SMILES: CCOc1nc(N2CC3(CCN3)C2)ncc1C(=O)Nc1cn2cc(C)nc2c(C)n1] |
| 25 | 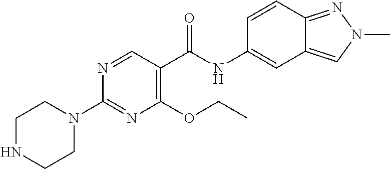 [SMILES: CCOc1nc(N2CCNCC2)ncc1C(=O)Nc1ccc2nn(C)cc2c1] |
| 26 | 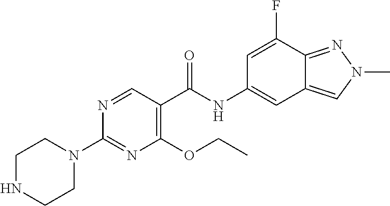 [SMILES: CCOc1nc(N2CCNCC2)ncc1C(=O)Nc1cc(F)c2nn(C)cc2c1] |
| 27 | 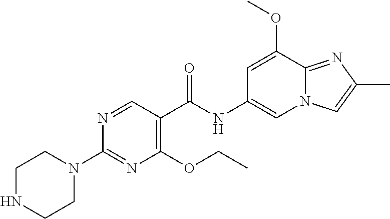 [SMILES: CCOc1nc(N2CCNCC2)ncc1C(=O)Nc1cc(OC)c2nc(C)cn2c1] |
| 28 | 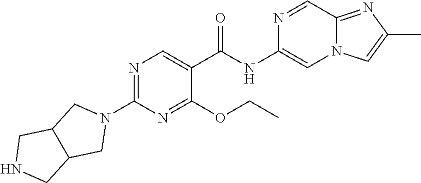 [SMILES: CCOc1nc(N2CC3CNCC3C2)ncc1C(=O)Nc1cn2cc(C)nc2cn1] |
| 29 | 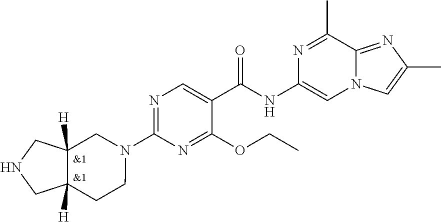 [SMILES: CCOc1nc(N2CC[C@@H]3CNC[C@@H]3C2)ncc1C(=O)Nc1cn2cc(C)nc2c(C)n1] |
| 30 | 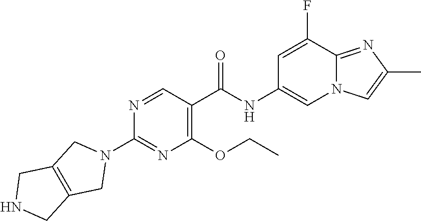 [SMILES: CCOc1nc(N2CC3=C(CNC3)C2)ncc1C(=O)Nc1cc(F)c2nc(C)cn2c1] |
| 31 | 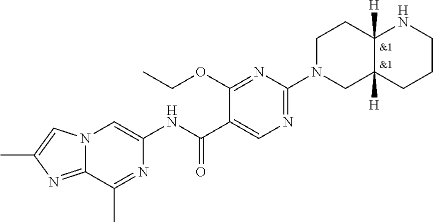 [SMILES: CCOc1nc(N2CC[C@@H]3NCCC[C@@H]3C2)ncc1C(=O)Nc1cn2cc(C)nc2c(C)n1] |
| 32 | 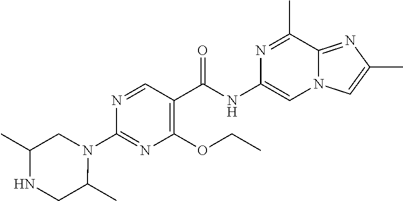 |
| 33 | 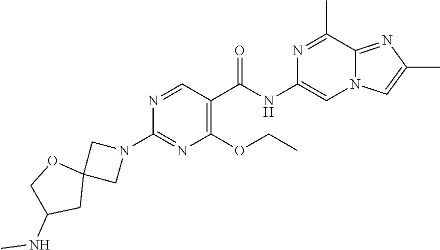 |
| 34 | 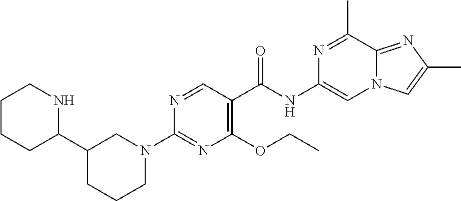 [SMILES: CCOc1nc(N2CCCC(C3CCCCN3)C2)ncc1C(=O)Nc1cn2cc(C)nc2c(C)n1] |
| 35 | 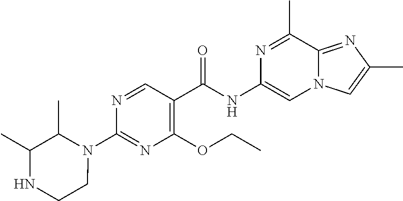 |
| 36 | 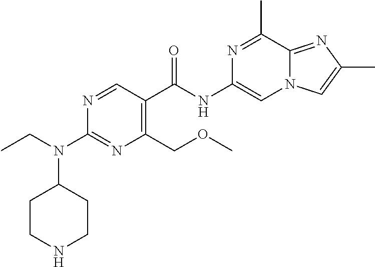 [SMILES: CCN(c1ncc(C(=O)Nc2cn3cc(C)nc3c(C)n2)c(COC)n1)C1CCNCC1] |
| 37 | 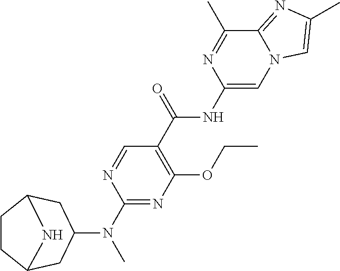 [SMILES: CCOc1nc(N(C)C2CC3CCC(C2)N3)ncc1C(=O)Nc1cn2cc(C)nc2c(C)n1] |
| 38 | 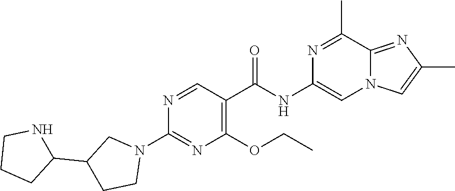 [SMILES: CCOc1nc(N2CCC(C3CCCN3)C2)ncc1C(=O)Nc1cn2cc(C)nc2c(C)n1] |
| 39 | 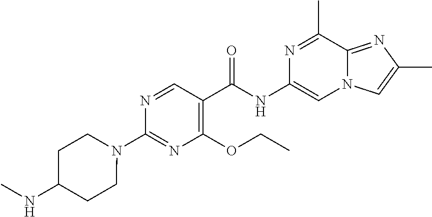 |
| 40 | 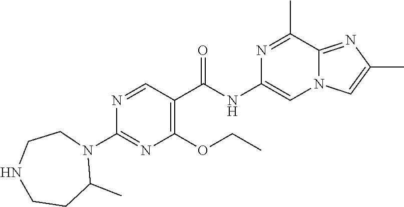 [SMILES: CCOc1nc(N2CCNCCC2C)ncc1C(=O)Nc1cn2cc(C)nc2c(C)n1] |
| 41 | 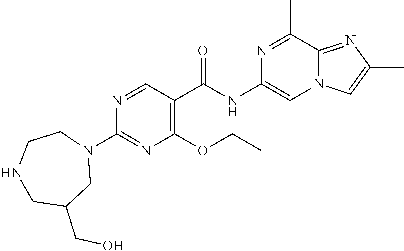 [SMILES: CCOc1nc(N2CCNCC(CO)C2)ncc1C(=O)Nc1cn2cc(C)nc2c(C)n1] |
| 42 | 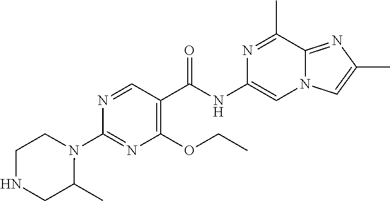 [SMILES: CCOc1nc(N2CCNCC2C)ncc1C(=O)Nc1cn2cc(C)nc2c(C)n1] |
| 43 | 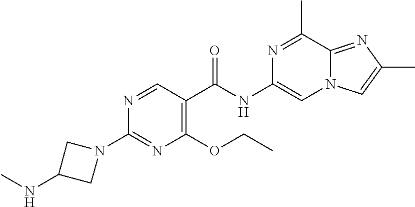 |
| 44 | 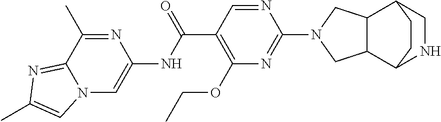 [SMILES: CCOc1nc(N2CC3C4CCC(NC4)C3C2)ncc1C(=O)Nc1cn2cc(C)nc2c(C)n1] |
| 45 | 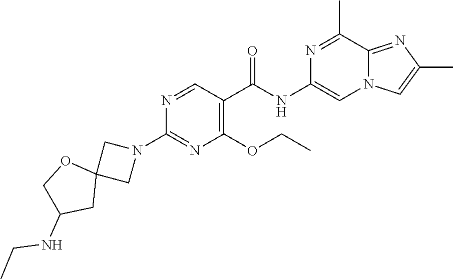 [SMILES: CCNC1COC2(C1)CN(c1ncc(C(=O)Nc3cn4cc(C)nc4c(C)n3)c(OCC)n1)C2] |
| 46 | 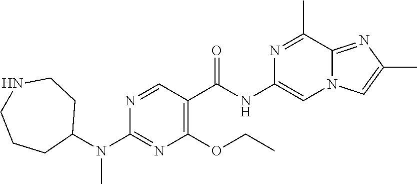 [SMILES: CCOc1nc(N(C)C2CCCNCC2)ncc1C(=O)Nc1cn2cc(C)nc2c(C)n1] |
| 47 | 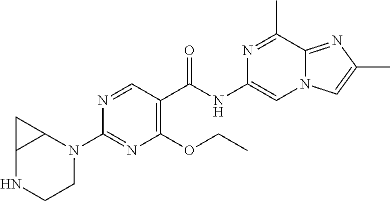 [SMILES: CCOc1nc(N2CCNC3CC32)ncc1C(=O)Nc1cn2cc(C)nc2c(C)n1] |
| 48 | 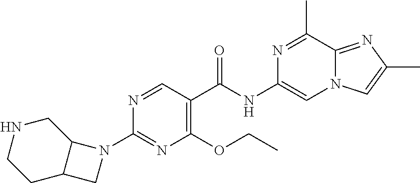 [SMILES: CCOc1nc(N2CC3CCNCC32)ncc1C(=O)Nc1cn2cc(C)nc2c(C)n1] |
| 49 | 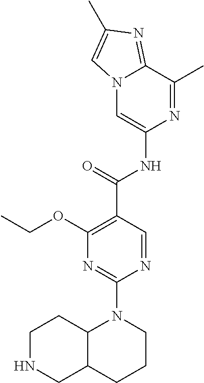 [SMILES: CCOc1nc(N2CCCC3CNCCC32)ncc1C(=O)Nc1cn2cc(C)nc2c(C)n1] |
| 50 | 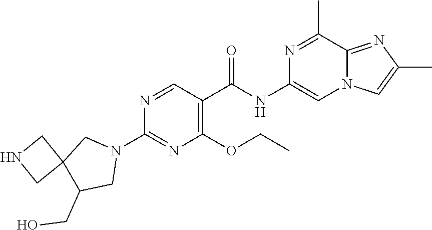 [SMILES: CCOc1nc(N2CC(CO)C3(CNC3)C2)ncc1C(=O)Nc1cn2cc(C)nc2c(C)n1] |
| 51 | 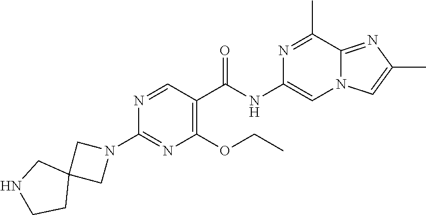 [SMILES: CCOc1nc(N2CC3(CCNC3)C2)ncc1C(=O)Nc1cn2cc(C)nc2c(C)n1] |
| 52 | 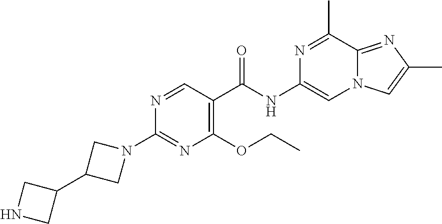 [SMILES: CCOc1nc(N2CC(C3CNC3)C2)ncc1C(=O)Nc1cn2cc(C)nc2c(C)n1] |
| 53 | 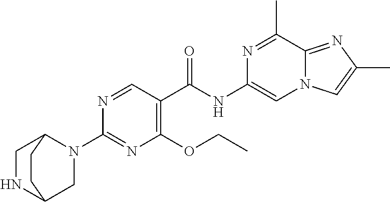 [SMILES: CCOc1nc(N2CC3CCC2CN3)ncc1C(=O)Nc1cn2cc(C)nc2c(C)n1] |
| 54 | 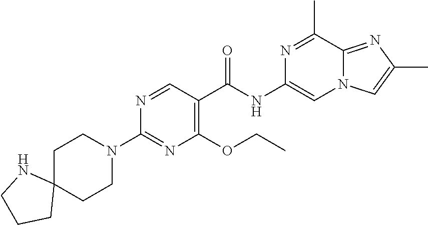 [SMILES: CCOc1nc(N2CCC3(CCCN3)CC2)ncc1C(=O)Nc1cn2cc(C)nc2c(C)n1] |
| 55 | 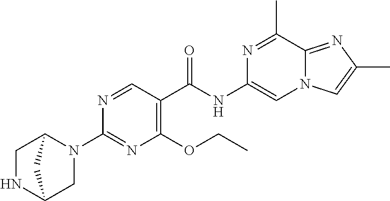 [SMILES: CCOc1nc(N2C[C@@H]3C[C@H]2CN3)ncc1C(=O)Nc1cn2cc(C)nc2c(C)n1] |
| 56 | 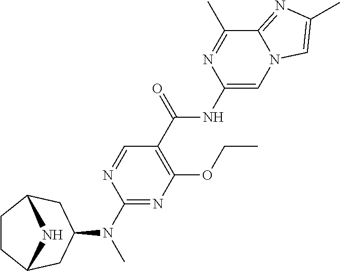 [SMILES: CCOc1nc(N(C)[C@@H]2C[C@H]3CC[C@@H](C2)N3)ncc1C(=O)Nc1cn2cc(C)nc2c(C)n1] |
| 57 | 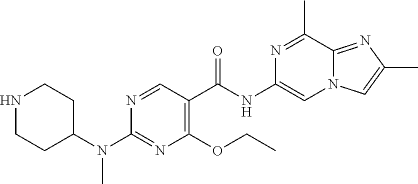 [SMILES: CCOc1nc(N(C)C2CCNCC2)ncc1C(=O)Nc1cn2cc(C)nc2c(C)n1] |
| 58 | 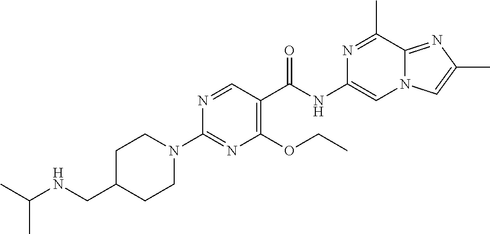 [SMILES: CCOc1nc(N2CCC(CNC(C)C)CC2)ncc1C(=O)Nc1cn2cc(C)nc2c(C)n1] |
| 59 | 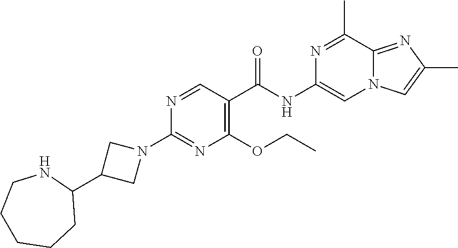 [SMILES: CCOc1nc(N2CC(C3CCCCCN3)C2)ncc1C(=O)Nc1cn2cc(C)nc2c(C)n1] |
| 60 | 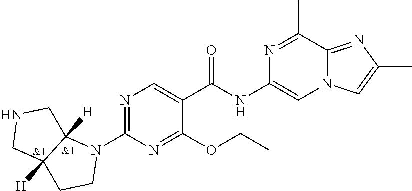 [SMILES: CCOc1nc(N2CC[C@@H]3CNC[C@@H]32)ncc1C(=O)Nc1cn2cc(C)nc2c(C)n1] |
| 61 | 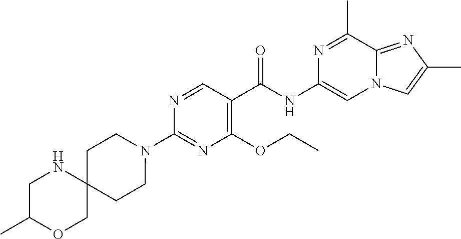 |
| 62 | 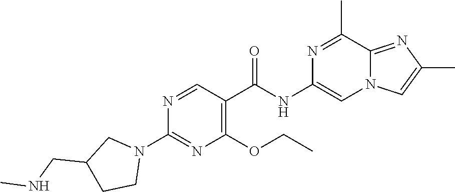 |
| 63 | 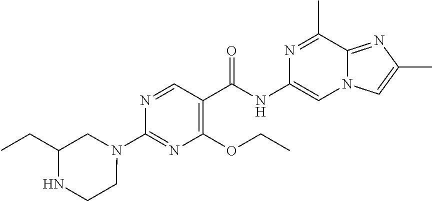 [SMILES: CCOc1nc(N2CCNC(CC)C2)ncc1C(=O)Nc1cn2cc(C)nc2c(C)n1] |
| 64 | 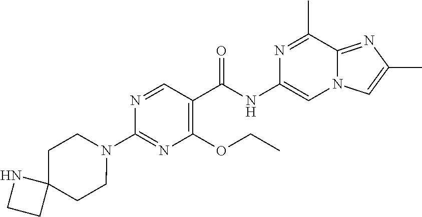 [SMILES: CCOc1nc(N2CCC3(CCN3)CC2)ncc1C(=O)Nc1cn2cc(C)nc2c(C)n1] |
| 65 | 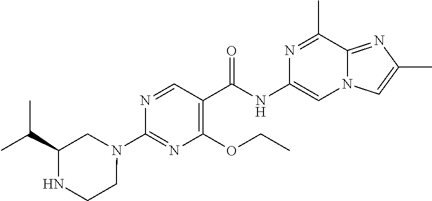 [SMILES: CCOc1nc(N2CCN[C@@H](C(C)C)C2)ncc1C(=O)Nc1cn2cc(C)nc2c(C)n1] |
| 66 | 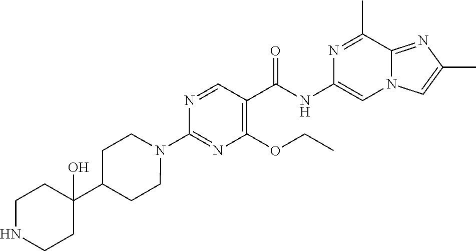 [SMILES: CCOc1nc(N2CCC(C3(O)CCNCC3)CC2)ncc1C(=O)Nc1cn2cc(C)nc2c(C)n1] |
| 67 | 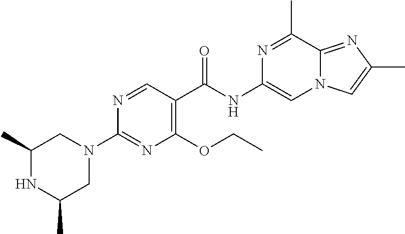 |
| 68 | 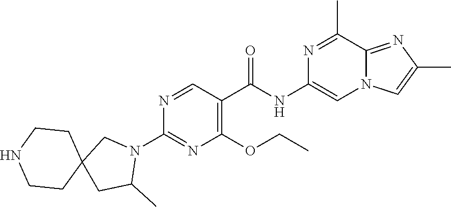 [SMILES: CCOc1nc(N2CC3(CCNCC3)CC2C)ncc1C(=O)Nc1cn2cc(C)nc2c(C)n1] |
| 69 | 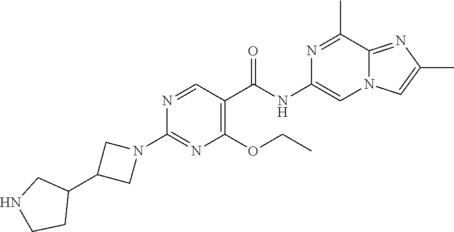 [SMILES: CCOc1nc(N2CC(C3CCNC3)C2)ncc1C(=O)Nc1cn2cc(C)nc2c(C)n1] |
| 70 | 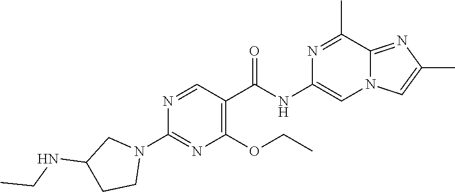 [SMILES: CCNC1CCN(c2ncc(C(=O)Nc3cn4cc(C)nc4c(C)n3)c(OCC)n2)C1] |
| 71 | 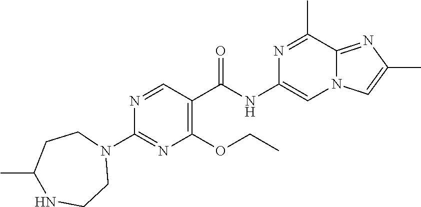 |
| 72 | 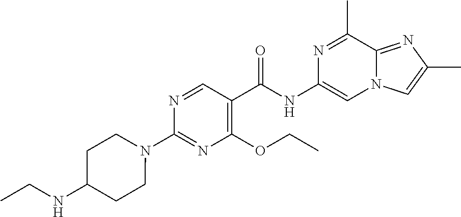 [SMILES: CCNC1CCN(c2ncc(C(=O)Nc3cn4cc(C)nc4c(C)n3)c(OCC)n2)CC1] |
| 73 | 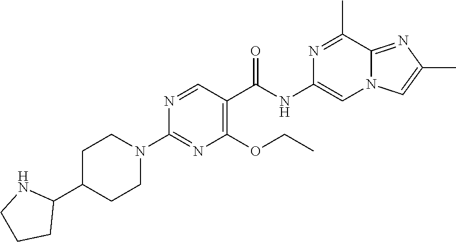 [SMILES: CCOc1nc(N2CCC(C3CCCN3)CC2)ncc1C(=O)Nc1cn2cc(C)nc2c(C)n1] |
| 74 | 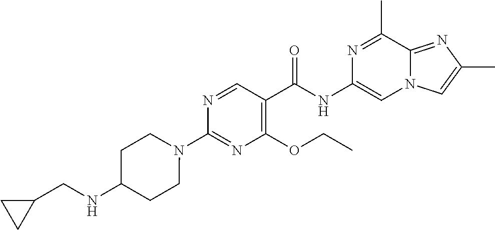 [SMILES: CCOc1nc(N2CCC(NCC3CC3)CC2)ncc1C(=O)Nc1cn2cc(C)nc2c(C)n1] |
| 75 | 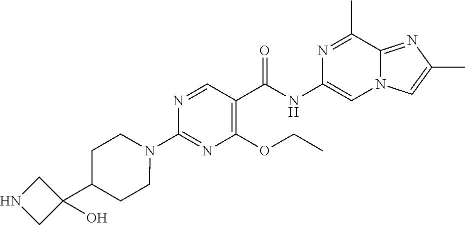 [SMILES: CCOc1nc(N2CCC(C3(O)CNC3)CC2)ncc1C(=O)Nc1cn2cc(C)nc2c(C)n1] |
| 76 | 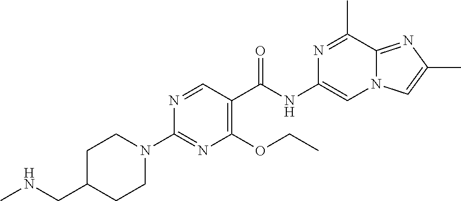 |
| 77 | 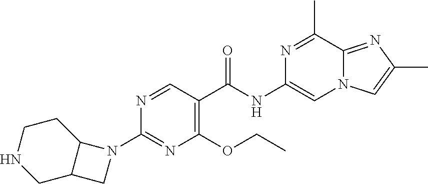 [SMILES: CCOc1nc(N2CC3CNCCC32)ncc1C(=O)Nc1cn2cc(C)nc2c(C)n1] |
| 78 | 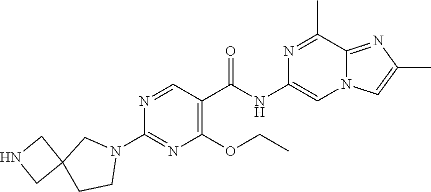 [SMILES: CCOc1nc(N2CCC3(CNC3)C2)ncc1C(=O)Nc1cn2cc(C)nc2c(C)n1] |
| 79 | 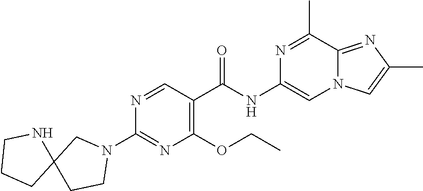 [SMILES: CCOc1nc(N2CCC3(CCCN3)C2)ncc1C(=O)Nc1cn2cc(C)nc2c(C)n1] |
| 80 | 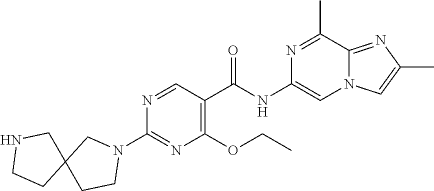 [SMILES: CCOc1nc(N2CCC3(CCNC3)C2)ncc1C(=O)Nc1cn2cc(C)nc2c(C)n1] |
| 81 | 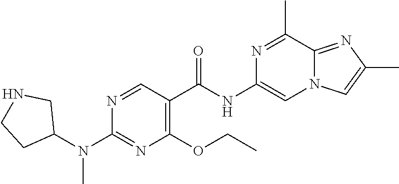 [SMILES: CCOc1nc(N(C)C2CCNC2)ncc1C(=O)Nc1cn2cc(C)nc2c(C)n1] |
| 82 | 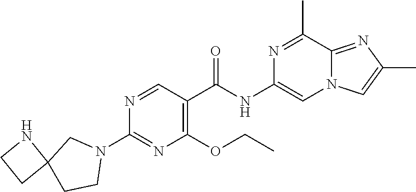 [SMILES: CCOc1nc(N2CCC3(CCN3)C2)ncc1C(=O)Nc1cn2cc(C)nc2c(C)n1] |
| 83 | 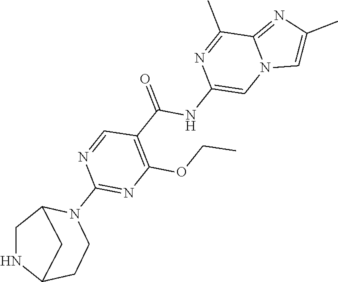 [SMILES: CCOc1nc(N2CCC3CC2CN3)ncc1C(=O)Nc1cn2cc(C)nc2c(C)n1] |
| 84 | 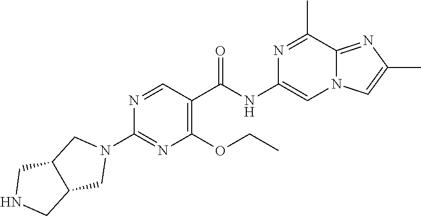 [SMILES: CCOc1nc(N2C[C@H]3CNC[C@H]3C2)ncc1C(=O)Nc1cn2cc(C)nc2c(C)n1] |
| 85 | 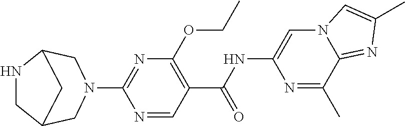 [SMILES: CCOc1nc(N2CC3CNC(C3)C2)ncc1C(=O)Nc1cn2cc(C)nc2c(C)n1] |
| 86 | 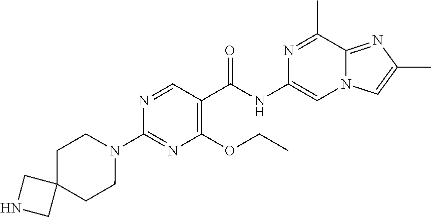 [SMILES: CCOc1nc(N2CCC3(CC2)CNC3)ncc1C(=O)Nc1cn2cc(C)nc2c(C)n1] |
| 87 | 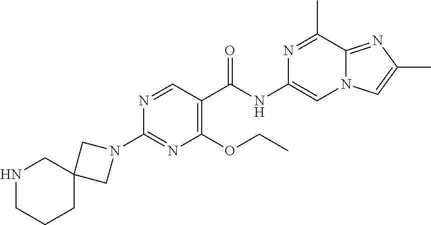 [SMILES: CCOc1nc(N2CC3(CCCNC3)C2)ncc1C(=O)Nc1cn2cc(C)nc2c(C)n1] |
| 88 | 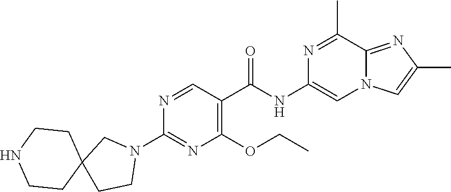 [SMILES: CCOc1nc(N2CCC3(CCNCC3)C2)ncc1C(=O)Nc1cn2cc(C)nc2c(C)n1] |
| 89 | 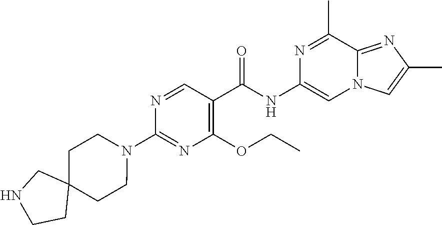 [SMILES: CCOc1nc(N2CCC3(CCNC3)CC2)ncc1C(=O)Nc1cn2cc(C)nc2c(C)n1] |
| 90 | 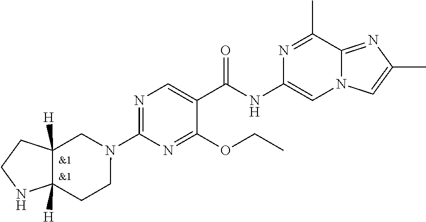 [SMILES: CCOc1nc(N2CC[C@@H]3NCC[C@@H]3C2)ncc1C(=O)Nc1cn2cc(C)nc2c(C)n1] |
| 91 | 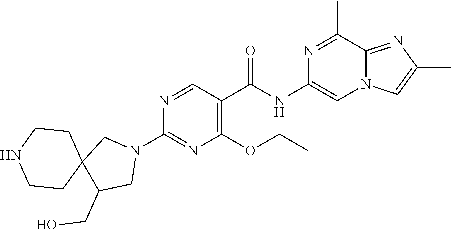 [SMILES: CCOc1nc(N2CC(CO)C3(CCNCC3)C2)ncc1C(=O)Nc1cn2cc(C)nc2c(C)n1] |
| 92 | 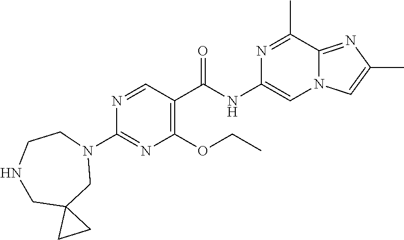 [SMILES: CCOc1nc(N2CCNCC3(CC3)C2)ncc1C(=O)Nc1cn2cc(C)nc2c(C)n1] |
| 93 | 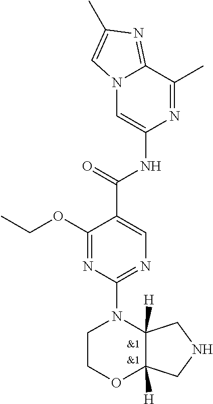 [SMILES: CCOc1nc(N2CCO[C@H]3CNC[C@H]32)ncc1C(=O)Nc1cn2cc(C)nc2c(C)n1] |
| 94 | 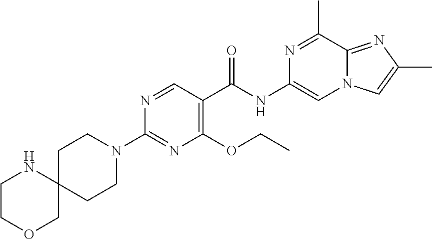 [SMILES: CCOc1nc(N2CCC3(CC2)COCCN3)ncc1C(=O)Nc1cn2cc(C)nc2c(C)n1] |
| 95 | 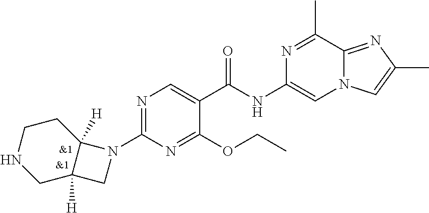 [SMILES: CCOc1nc(N2C[C@H]3CNCC[C@H]32)ncc1C(=O)Nc1cn2cc(C)nc2c(C)n1] |
| 96 | 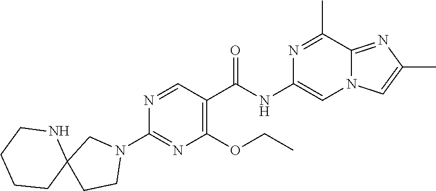 [SMILES: CCOc1nc(N2CCC3(CCCCN3)C2)ncc1C(=O)Nc1cn2cc(C)nc2c(C)n1] |
| 97 | 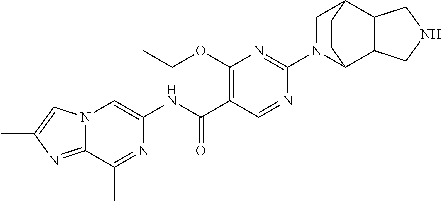 |
| 98 | 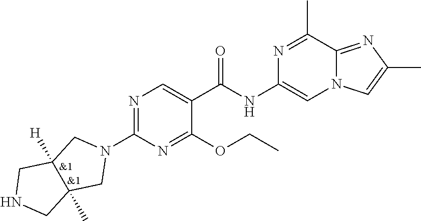 [SMILES: CCOc1nc(N2C[C@@H]3CNC[C@]3(C)C2)ncc1C(=O)Nc1cn2cc(C)nc2c(C)n1] |
| 99 | 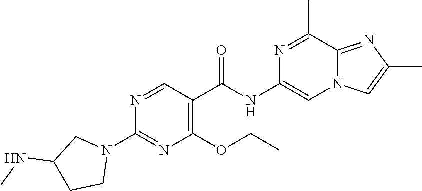 |
| 100 | 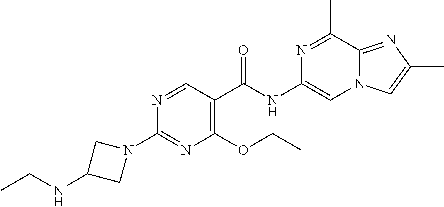 [SMILES: CCNC1CN(c2ncc(C(=O)Nc3cn4cc(C)nc4c(C)n3)c(OCC)n2)C1] |
| 101 | 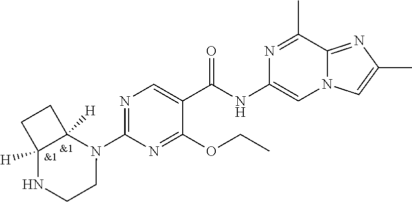 [SMILES: CCOc1nc(N2CCN[C@H]3CC[C@H]32)ncc1C(=O)Nc1cn2cc(C)nc2c(C)n1] |
| 102 | 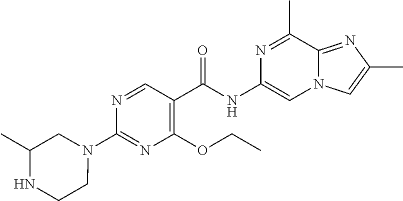 |
| 103 | 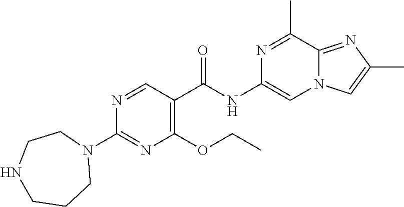 [SMILES: CCOc1nc(N2CCCNCC2)ncc1C(=O)Nc1cn2cc(C)nc2c(C)n1] |
| 104 | 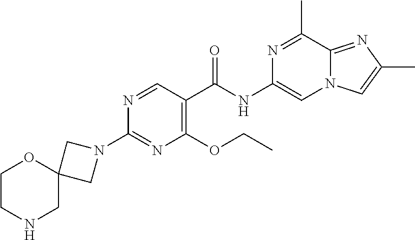 [SMILES: CCOc1nc(N2CC3(CNCCO3)C2)ncc1C(=O)Nc1cn2cc(C)nc2c(C)n1] |
| 105 | 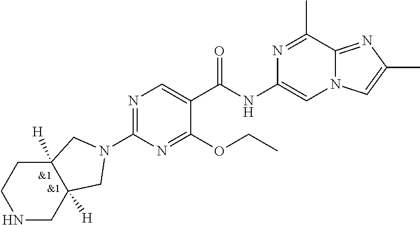 [SMILES: CCOc1nc(N2C[C@H]3CNCC[C@H]3C2)ncc1C(=O)Nc1cn2cc(C)nc2c(C)n1] |
| 106 | 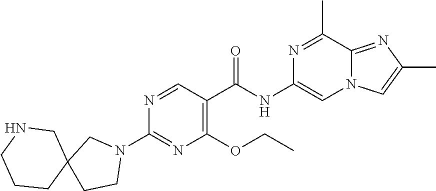 [SMILES: CCOc1nc(N2CCC3(CCCNC3)C2)ncc1C(=O)Nc1cn2cc(C)nc2c(C)n1] |
| 107 | 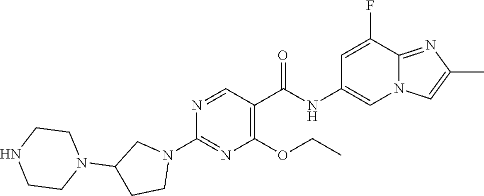 [SMILES: CCOc1nc(N2CCC(N3CCNCC3)C2)ncc1C(=O)Nc1cc(F)c2nc(C)cn2c1] |
| 108 | 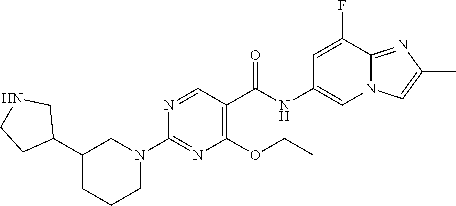 [SMILES: CCOc1nc(N2CCCC(C3CCNC3)C2)ncc1C(=O)Nc1cc(F)c2nc(C)cn2c1] |
| 109 | 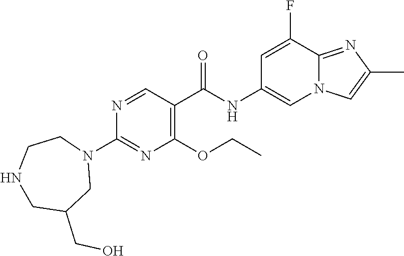 [SMILES: CCOc1nc(N2CCNCC(CO)C2)ncc1C(=O)Nc1cc(F)c2nc(C)cn2c1] |
| 110 | 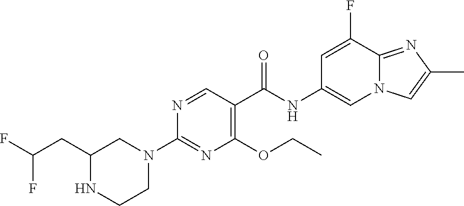 [SMILES: CCOc1nc(N2CCNC(CC(F)F)C2)ncc1C(=O)Nc1cc(F)c2nc(C)cn2c1] |
| 111 | 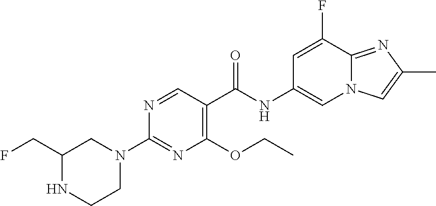 [SMILES: CCOc1nc(N2CCNC(CF)C2)ncc1C(=O)Nc1cc(F)c2nc(C)cn2c1] |
| 112 | 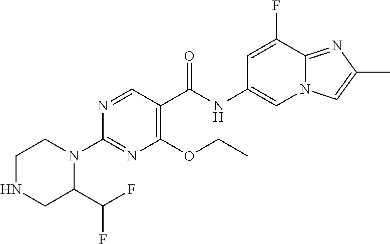 [SMILES: CCOc1nc(N2CCNCC2C(F)F)ncc1C(=O)Nc1cc(F)c2nc(C)cn2c1] |
| 113 | 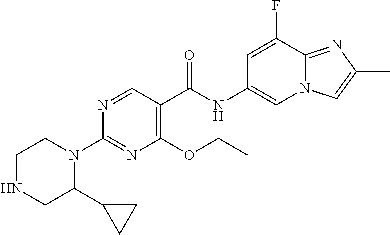 [SMILES: CCOc1nc(N2CCNCC2C2CC2)ncc1C(=O)Nc1cc(F)c2nc(C)cn2c1] |
| 114 | 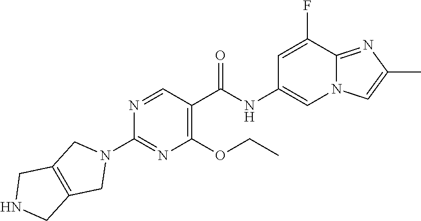 [SMILES: CCOc1nc(N2CC3=C(CNC3)C2)ncc1C(=O)Nc1cc(F)c2nc(C)cn2c1] |
| 115 | 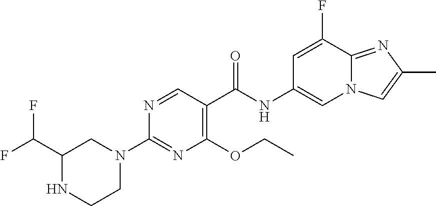 [SMILES: CCOc1nc(N2CCNC(C(F)F)C2)ncc1C(=O)Nc1cc(F)c2nc(C)cn2c1] |
| 116 | 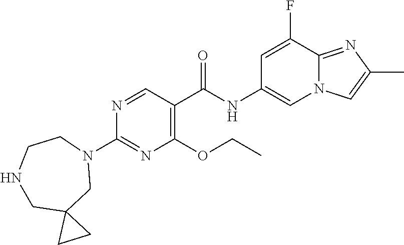 [SMILES: CCOc1nc(N2CCNCC3(CC3)C2)ncc1C(=O)Nc1cc(F)c2nc(C)cn2c1] |
| 117 | 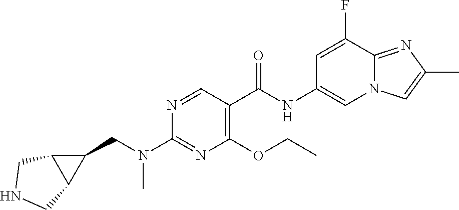 [SMILES: CCOc1nc(N(C)C[C@H]2[C@@H]3CNC[C@@H]32)ncc1C(=O)Nc1cc(F)c2nc(C)cn2c1] |
| 118 | 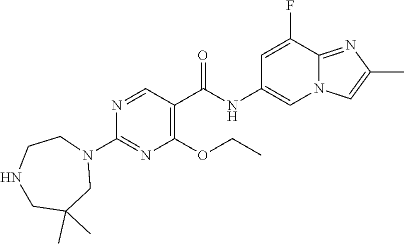 [SMILES: CCOc1nc(N2CCNCC(C)(C)C2)ncc1C(=O)Nc1cc(F)c2nc(C)cn2c1] |
| 119 | 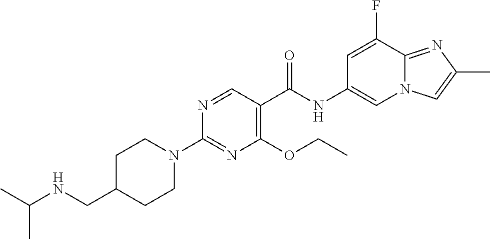 [SMILES: CCOc1nc(N2CCC(CNC(C)C)CC2)ncc1C(=O)Nc1cc(F)c2nc(C)cn2c1] |
| 120 | 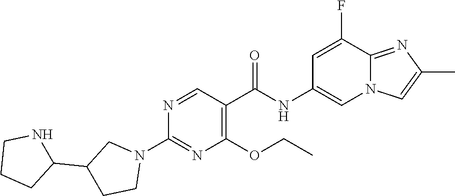 [SMILES: CCOc1nc(N2CCC(C3CCCN3)C2)ncc1C(=O)Nc1cc(F)c2nc(C)cn2c1] |
| 121 | 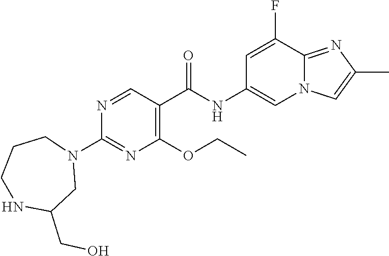 [SMILES: CCOc1nc(N2CCCNC(CO)C2)ncc1C(=O)Nc1cc(F)c2nc(C)cn2c1] |
| 122 | 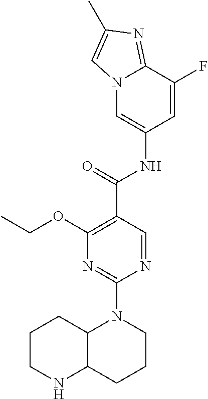 [SMILES: CCOc1nc(N2CCCC3NCCCC32)ncc1C(=O)Nc1cc(F)c2nc(C)cn2c1] |
| 123 | 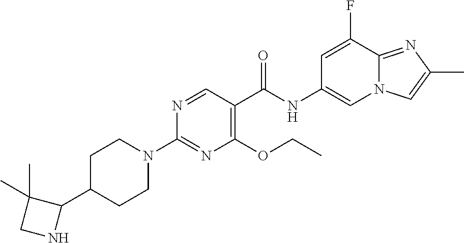 |
| 124 | 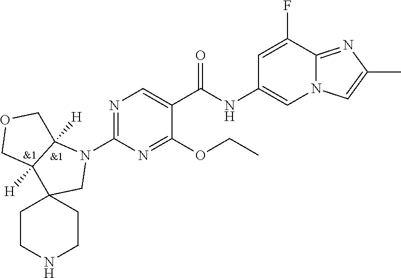 [SMILES: CCOc1nc(N2CC3(CCNCC3)[C@H]3COC[C@H]32)ncc1C(=O)Nc1cc(F)c2nc(C)cn2c1] |
| 125 | 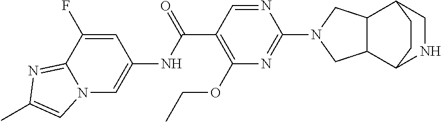 [SMILES: CCOc1nc(N2CC3C4CCC(NC4)C3C2)ncc1C(=O)Nc1cc(F)c2nc(C)cn2c1] |
| 126 | 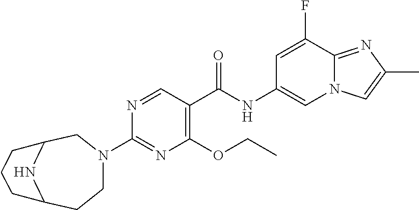 [SMILES: CCOc1nc(N2CCC3CCCC(C2)N3)ncc1C(=O)Nc1cc(F)c2nc(C)cn2c1] |
| 127 | 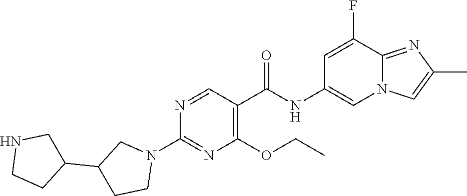 [SMILES: CCOc1nc(N2CCC(C3CCNC3)C2)ncc1C(=O)Nc1cc(F)c2nc(C)cn2c1] |
| 128 | 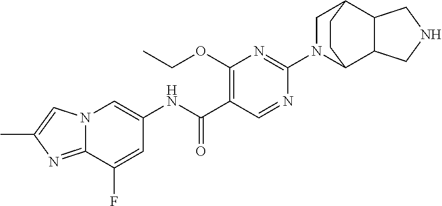 [SMILES: CCOc1nc(N2CC3CCC2C2CNCC32)ncc1C(=O)Nc1cc(F)c2nc(C)cn2c1] |
| 129 | 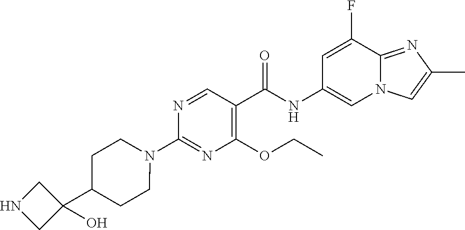 [SMILES: CCOc1nc(N2CCC(C3(O)CNC3)CC2)ncc1C(=O)Nc1cc(F)c2nc(C)cn2c1] |
| 130 | 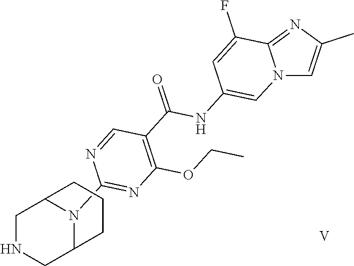 |
| 131 | 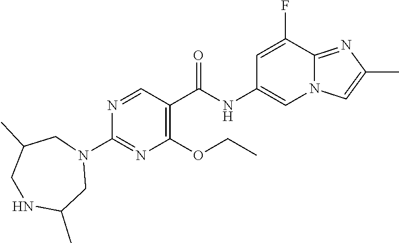 |
| 132 | 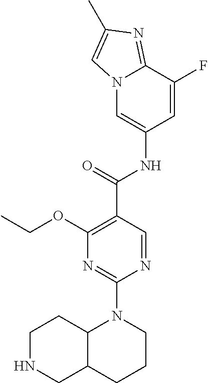 [SMILES: CCOc1nc(N2CCCC3CNCCC32)ncc1C(=O)Nc1cc(F)c2nc(C)cn2c1] |
| 133 | 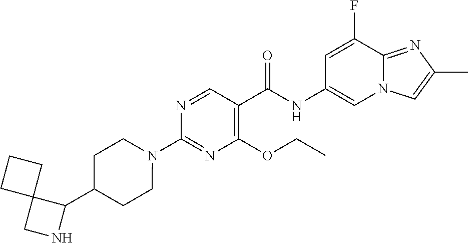 [SMILES: CCOc1nc(N2CCC(C3NCC34CCC4)CC2)ncc1C(=O)Nc1cc(F)c2nc(C)cn2c1] |
| 134 | 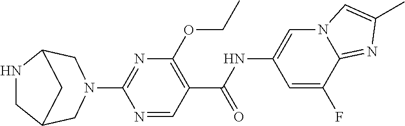 [SMILES: CCOc1nc(N2CC3CNC(C3)C2)ncc1C(=O)Nc1cc(F)c2nc(C)cn2c1] |
| 135 | 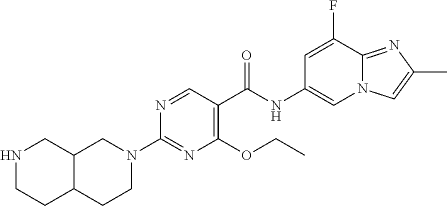 [SMILES: CCOc1nc(N2CCC3CCNCC3C2)ncc1C(=O)Nc1cc(F)c2nc(C)cn2c1] |
| 136 | 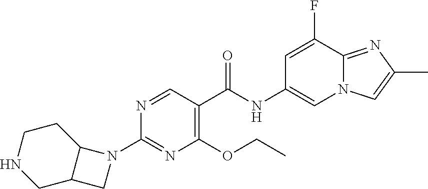 [SMILES: CCOc1nc(N2CC3CNCCC32)ncc1C(=O)Nc1cc(F)c2nc(C)cn2c1] |
| 137 | 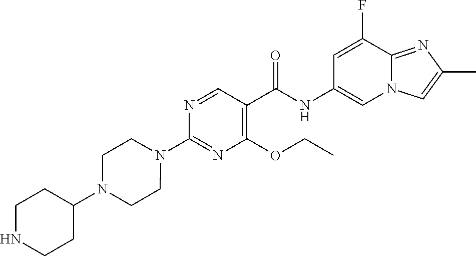 [SMILES: CCOc1nc(N2CCN(C3CCNCC3)CC2)ncc1C(=O)Nc1cc(F)c2nc(C)cn2c1] |
| 138 | 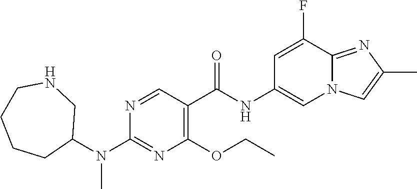 [SMILES: CCOc1nc(N(C)C2CCCCNC2)ncc1C(=O)Nc1cc(F)c2nc(C)cn2c1] |
| 139 | 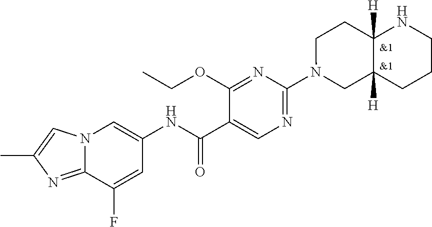 [SMILES: CCOc1nc(N2CC[C@@H]3NCCC[C@@H]3C2)ncc1C(=O)Nc1cc(F)c2nc(C)cn2c1] |
| 140 | 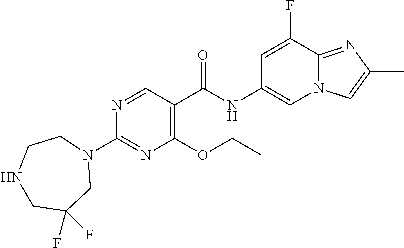 [SMILES: CCOc1nc(N2CCNCC(F)(F)C2)ncc1C(=O)Nc1cc(F)c2nc(C)cn2c1] |
| 141 | 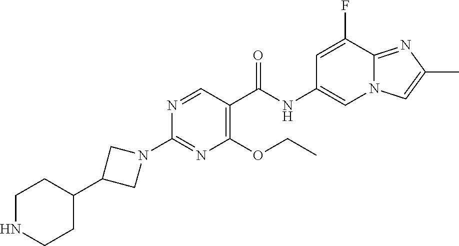 [SMILES: CCOc1nc(N2CC(C3CCNCC3)C2)ncc1C(=O)Nc1cc(F)c2nc(C)cn2c1] |
| 142 | 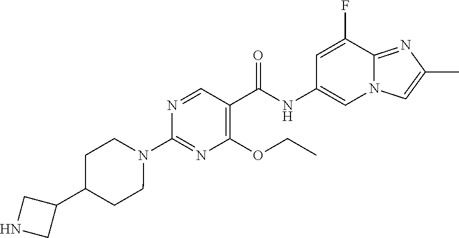 [SMILES: CCOc1nc(N2CCC(C3CNC3)CC2)ncc1C(=O)Nc1cc(F)c2nc(C)cn2c1] |
| 143 | 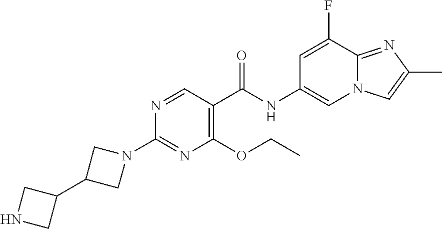 [SMILES: CCOc1nc(N2CC(C3CNC3)C2)ncc1C(=O)Nc1cc(F)c2nc(C)cn2c1] |
| 144 | 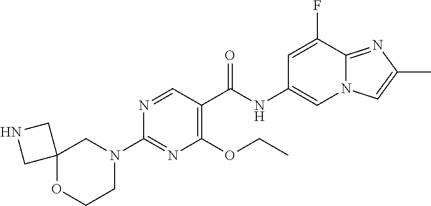 [SMILES: CCOc1nc(N2CCOC3(CNC3)C2)ncc1C(=O)Nc1cc(F)c2nc(C)cn2c1] |
| 145 | 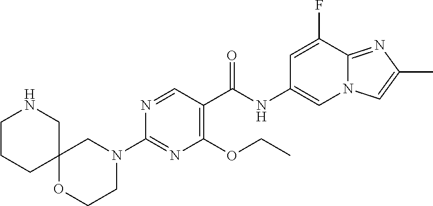 [SMILES: CCOc1nc(N2CCOC3(CCCNC3)C2)ncc1C(=O)Nc1cc(F)c2nc(C)cn2c1] |
| 146 | 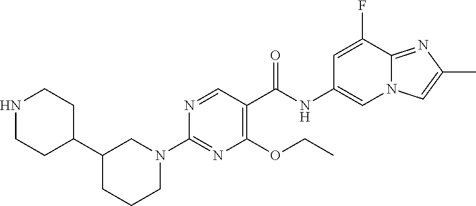 [SMILES: CCOc1nc(N2CCCC(C3CCNCC3)C2)ncc1C(=O)Nc1cc(F)c2nc(C)cn2c1] |
| 147 | 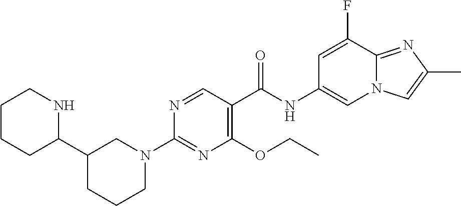 [SMILES: CCOc1nc(N2CCCC(C3CCCCN3)C2)ncc1C(=O)Nc1cc(F)c2nc(C)cn2c1] |
| 148 | 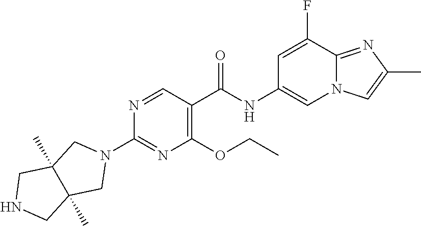 [SMILES: CCOc1nc(N2C[C@]3(C)CNC[C@]3(C)C2)ncc1C(=O)Nc1cc(F)c2nc(C)cn2c1] |
| 149 | 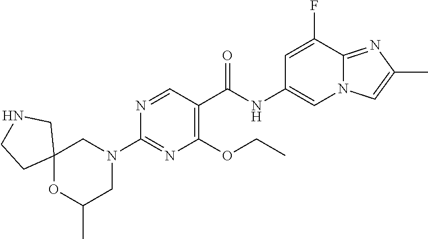 [SMILES: CCOc1nc(N2CC(C)OC3(CCNC3)C2)ncc1C(=O)Nc1cc(F)c2nc(C)cn2c1] |
| 150 | 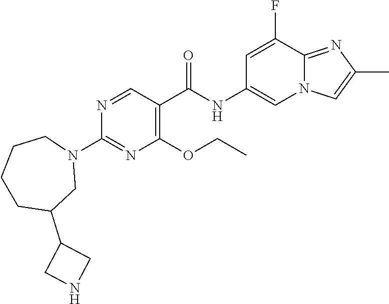 [SMILES: CCOc1nc(N2CCCCC(C3CNC3)C2)ncc1C(=O)Nc1cc(F)c2nc(C)cn2c1] |
| 151 | 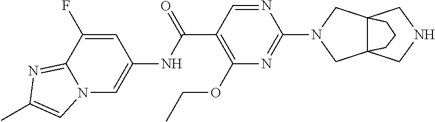 [SMILES: CCOc1nc(N2CC34CCCC3(CNC4)C2)ncc1C(=O)Nc1cc(F)c2nc(C)cn2c1] |
| 152 | 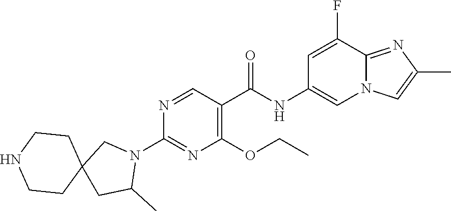 [SMILES: CCOc1nc(N2CC3(CCNCC3)CC2C)ncc1C(=O)Nc1cc(F)c2nc(C)cn2c1] |
| 153 | 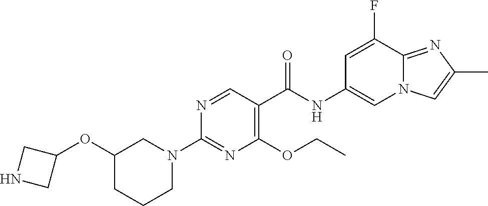 [SMILES: CCOc1nc(N2CCCC(OC3CNC3)C2)ncc1C(=O)Nc1cc(F)c2nc(C)cn2c1] |
| 154 | 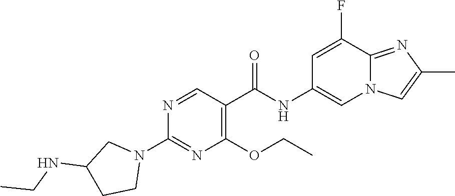 [SMILES: CCNC1CCN(c2ncc(C(=O)Nc3cc(F)c4nc(C)cn4c3)c(OCC)n2)C1] |
| 155 | 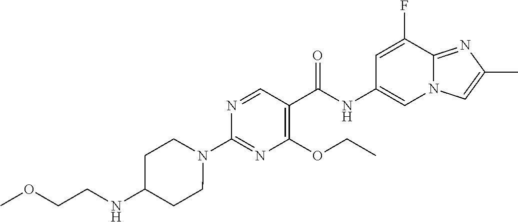 |
| 156 | 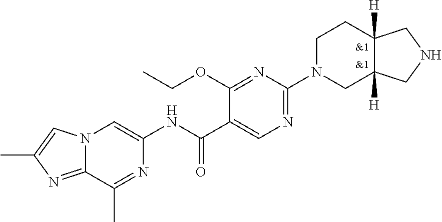 [SMILES: CCOc1nc(N2CC[C@@H]3CNC[C@@H]3C2)ncc1C(=O)Nc1cn2cc(C)nc2c(C)n1] |
| 157 | 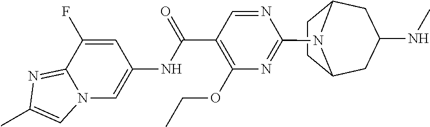 |
| 158 | 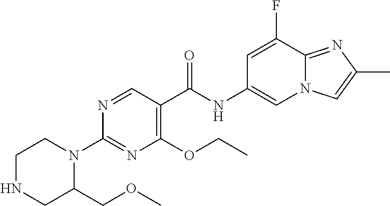 [SMILES: CCOc1nc(N2CCNCC2COC)ncc1C(=O)Nc1cc(F)c2nc(C)cn2c1] |
| 159 | 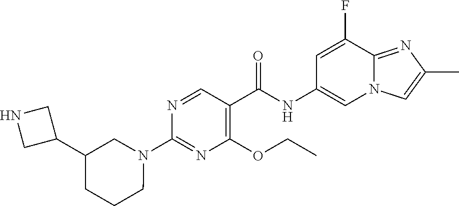 [SMILES: CCOc1nc(N2CCCC(C3CNC3)C2)ncc1C(=O)Nc1cc(F)c2nc(C)cn2c1] |
| 160 | 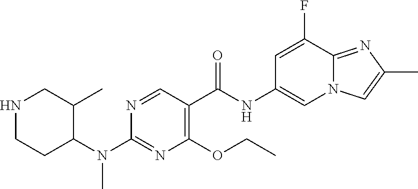 [SMILES: CCOc1nc(N(C)C2CCNCC2C)ncc1C(=O)Nc1cc(F)c2nc(C)cn2c1] |
| 161 | 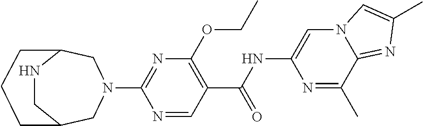 [SMILES: CCOc1nc(N2CC3CCCC(C2)NC3)ncc1C(=O)Nc1cn2cc(C)nc2c(C)n1] |
| 162 | 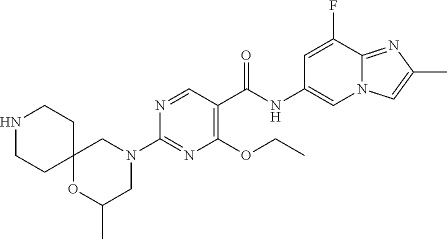 [SMILES: CCOc1nc(N2CC(C)OC3(CCNCC3)C2)ncc1C(=O)Nc1cc(F)c2nc(C)cn2c1] |
| 163 | 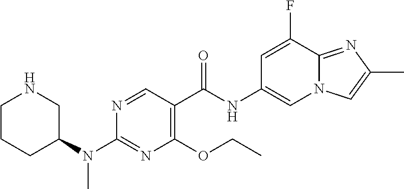 [SMILES: CCOc1nc(N(C)[C@H]2CCCNC2)ncc1C(=O)Nc1cc(F)c2nc(C)cn2c1] |
| 164 | 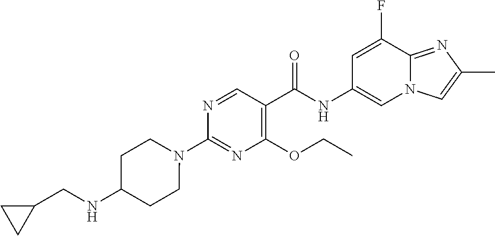 [SMILES: CCOc1nc(N2CCC(NCC3CC3)CC2)ncc1C(=O)Nc1cc(F)c2nc(C)cn2c1] |
| 165 | 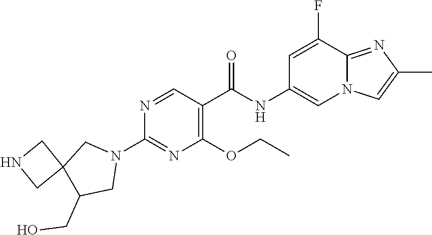 [SMILES: CCOc1nc(N2CC(CO)C3(CNC3)C2)ncc1C(=O)Nc1cc(F)c2nc(C)cn2c1] |
| 166 | 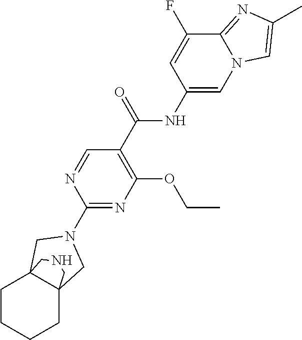 [SMILES: CCOc1nc(N2CC34CCCCC3(CNC4)C2)ncc1C(=O)Nc1cc(F)c2nc(C)cn2c1] |
| 167 | 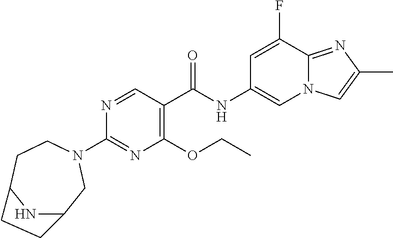 [SMILES: CCOc1nc(N2CCC3CCC(C2)N3)ncc1C(=O)Nc1cc(F)c2nc(C)cn2c1] |
| 168 | 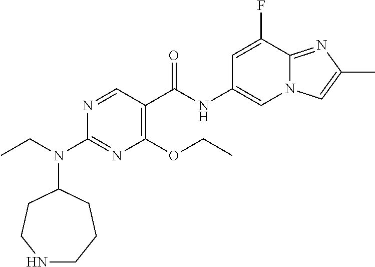 [SMILES: CCOc1nc(N(CC)C2CCCNCC2)ncc1C(=O)Nc1cc(F)c2nc(C)cn2c1] |
| 169 | 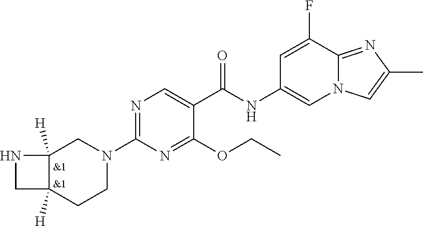 [SMILES: CCOc1nc(N2CC[C@H]3CN[C@H]3C2)ncc1C(=O)Nc1cc(F)c2nc(C)cn2c1] |
| 170 | 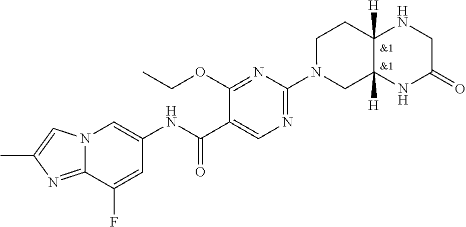 [SMILES: CCOc1nc(N2CC[C@@H]3NCC(=O)N[C@@H]3C2)ncc1C(=O)Nc1cc(F)c2nc(C)cn2c1] |
| 171 | 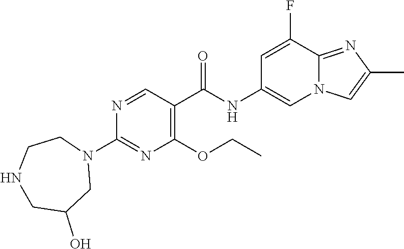 [SMILES: CCOc1nc(N2CCNCC(O)C2)ncc1C(=O)Nc1cc(F)c2nc(C)cn2c1] |
| 172 | 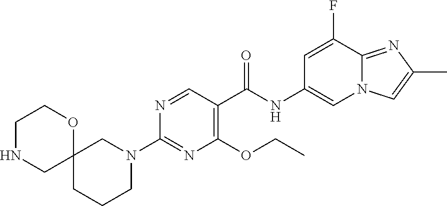 [SMILES: CCOc1nc(N2CCCC3(CNCCO3)C2)ncc1C(=O)Nc1cc(F)c2nc(C)cn2c1] |
| 173 | 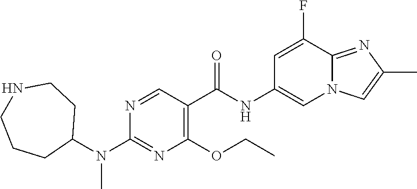 [SMILES: CCOc1nc(N(C)C2CCCNCC2)ncc1C(=O)Nc1cc(F)c2nc(C)cn2c1] |
| 174 | 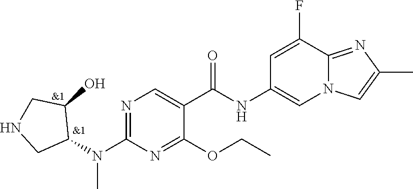 [SMILES: CCOc1nc(N(C)[C@@H]2CNC[C@H]2O)ncc1C(=O)Nc1cc(F)c2nc(C)cn2c1] |
| 175 | 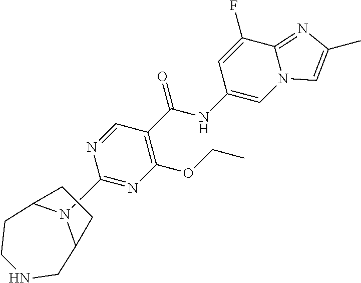 [SMILES: CCOc1nc(N2C3CCNCC2CC3)ncc1C(=O)Nc1cc(F)c2nc(C)cn2c1] |
| 176 | 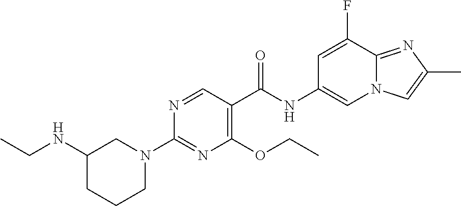 [SMILES: CCNC1CCCN(c2ncc(C(=O)Nc3cc(F)c4nc(C)cn4c3)c(OCC)n2)C1] |
| 177 | 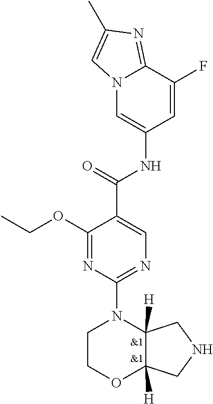 [SMILES: CCOc1nc(N2CCO[C@H]3CNC[C@H]32)ncc1C(=O)Nc1cc(F)c2nc(C)cn2c1] |
| 178 | 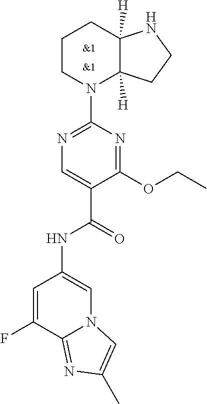 [SMILES: CCOc1nc(N2CCC[C@H]3NCC[C@H]32)ncc1C(=O)Nc1cc(F)c2nc(C)cn2c1] |
| 179 | 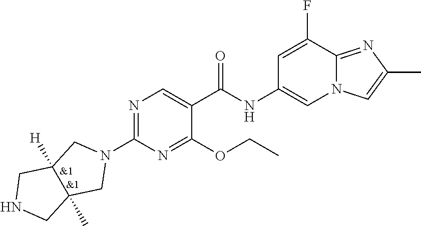 [SMILES: CCOc1nc(N2C[C@@H]3CNC[C@]3(C)C2)ncc1C(=O)Nc1cc(F)c2nc(C)cn2c1] |
| 180 | 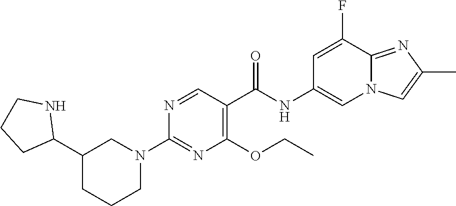 [SMILES: CCOc1nc(N2CCCC(C3CCCN3)C2)ncc1C(=O)Nc1cc(F)c2nc(C)cn2c1] |
| 181 | 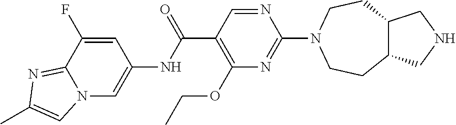 [SMILES: CCOc1nc(N2CC[C@@H]3CNC[C@@H]3CC2)ncc1C(=O)Nc1cc(F)c2nc(C)cn2c1] |
| 182 | 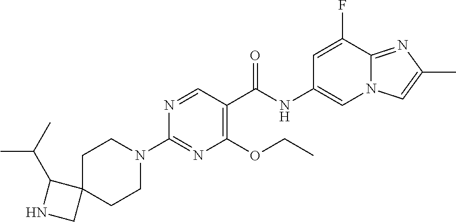 [SMILES: CCOc1nc(N2CCC3(CC2)CNC3C(C)C)ncc1C(=O)Nc1cc(F)c2nc(C)cn2c1] |
| 183 | 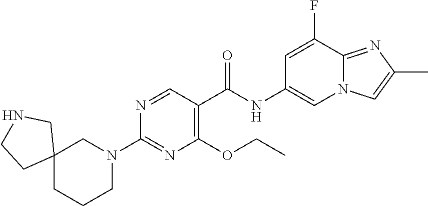 [SMILES: CCOc1nc(N2CCCC3(CCNC3)C2)ncc1C(=O)Nc1cc(F)c2nc(C)cn2c1] |
| 184 | 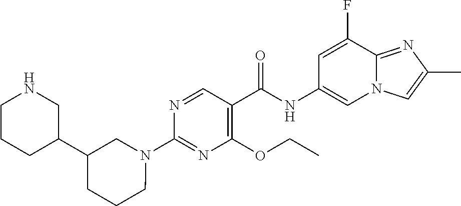 [SMILES: CCOc1nc(N2CCCC(C3CCCNC3)C2)ncc1C(=O)Nc1cc(F)c2nc(C)cn2c1] |
| 185 | 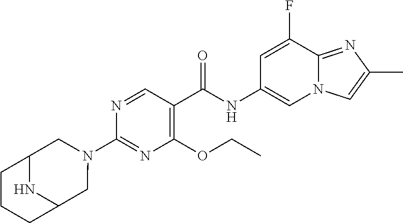 [SMILES: CCOc1nc(N2CC3CCCC(C2)N3)ncc1C(=O)Nc1cc(F)c2nc(C)cn2c1] |
| 186 | 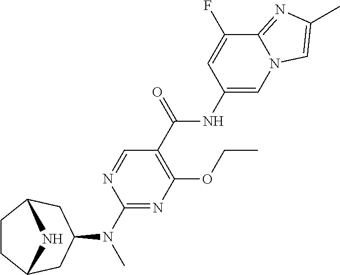 [SMILES: CCOc1nc(N(C)[C@@H]2C[C@H]3CC[C@@H](C2)N3)ncc1C(=O)Nc1cc(F)c2nc(C)cn2c1] |
| 187 | 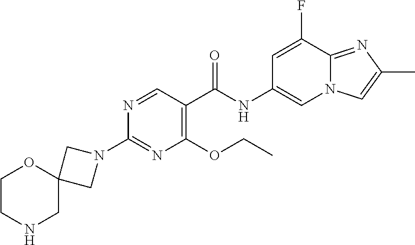 [SMILES: CCOc1nc(N2CC3(CNCCO3)C2)ncc1C(=O)Nc1cc(F)c2nc(C)cn2c1] |
| 188 | 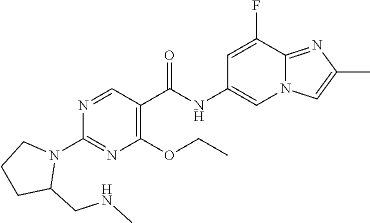 [SMILES: CCOc1nc(N2CCCC2CNC)ncc1C(=O)Nc1cc(F)c2nc(C)cn2c1] |
| 189 | 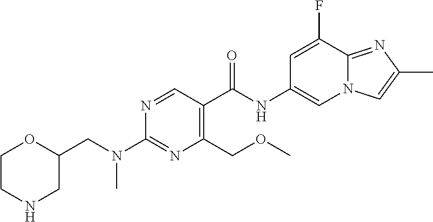 [SMILES: COCc1nc(N(C)CC2CNCCO2)ncc1C(=O)Nc1cc(F)c2nc(C)cn2c1] |
| 190 | 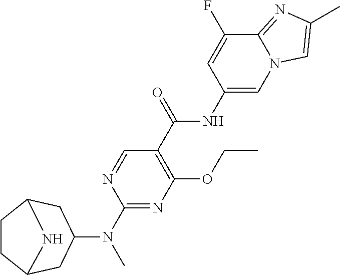 [SMILES: CCOc1nc(N(C)C2CC3CCC(C2)N3)ncc1C(=O)Nc1cc(F)c2nc(C)cn2c1] |
| 191 | 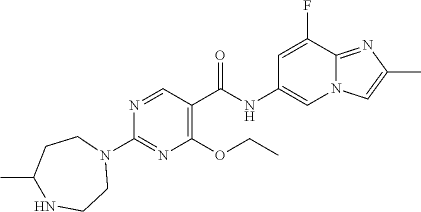 |
| 192 | 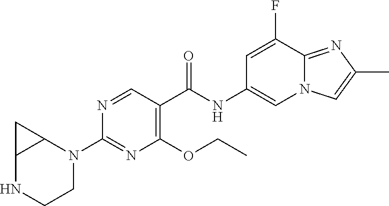 [SMILES: CCOc1nc(N2CCNC3CC32)ncc1C(=O)Nc1cc(F)c2nc(C)cn2c1] |
| 193 | 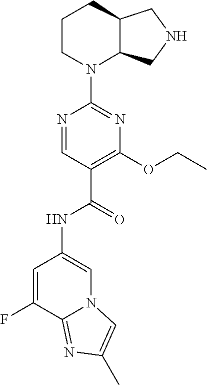 [SMILES: CCOc1nc(N2CCC[C@H]3CNC[C@H]32)ncc1C(=O)Nc1cc(F)c2nc(C)cn2c1] |
| 194 | 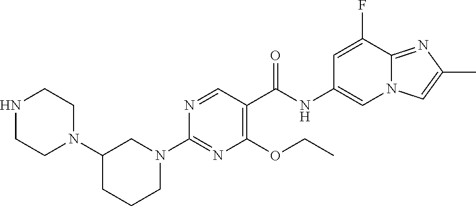 [SMILES: CCOc1nc(N2CCCC(N3CCNCC3)C2)ncc1C(=O)Nc1cc(F)c2nc(C)cn2c1] |
| 195 | 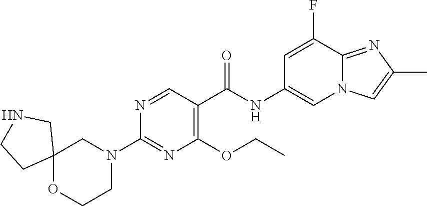 [SMILES: CCOc1nc(N2CCOC3(CCNC3)C2)ncc1C(=O)Nc1cc(F)c2nc(C)cn2c1] |
| 196 | 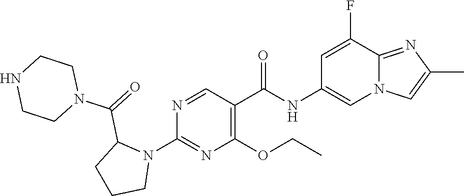 [SMILES: CCOc1nc(N2CCCC2C(=O)N2CCNCC2)ncc1C(=O)Nc1cc(F)c2nc(C)cn2c1] |
| 197 | 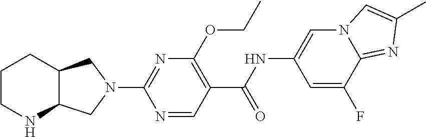 [SMILES: CCOc1nc(N2C[C@@H]3CCCN[C@@H]3C2)ncc1C(=O)Nc1cc(F)c2nc(C)cn2c1] |
| 198 | 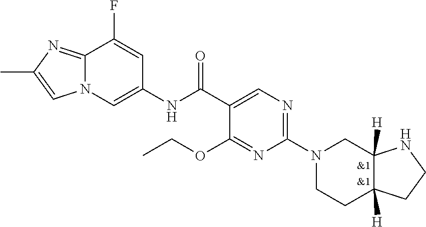 [SMILES: CCOc1nc(N2CC[C@H]3CCN[C@H]3C2)ncc1C(=O)Nc1cc(F)c2nc(C)cn2c1] |
| 199 | 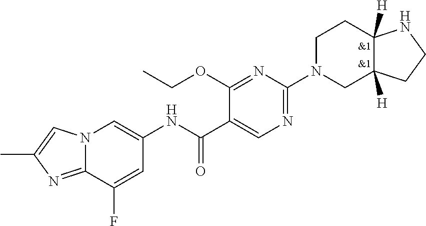 [SMILES: CCOc1nc(N2CC[C@@H]3NCC[C@@H]3C2)ncc1C(=O)Nc1cc(F)c2nc(C)cn2c1] |
| 200 | 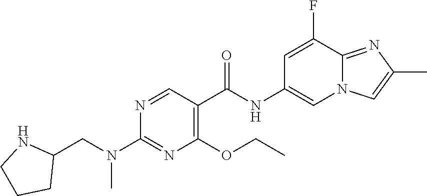 [SMILES: CCOc1nc(N(C)CC2CCCN2)ncc1C(=O)Nc1cc(F)c2nc(C)cn2c1] |
| 201 | 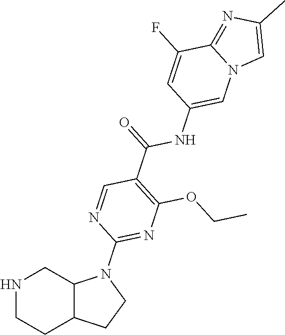 [SMILES: CCOc1nc(N2CCC3CCNCC32)ncc1C(=O)Nc1cc(F)c2nc(C)cn2c1] |
| 202 | 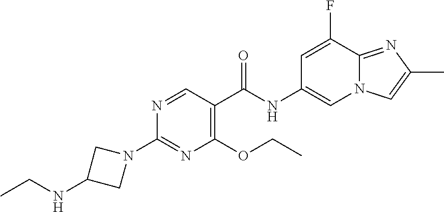 [SMILES: CCNC1CN(c2ncc(C(=O)Nc3cc(F)c4nc(C)cn4c3)c(OCC)n2)C1] |
| 203 | 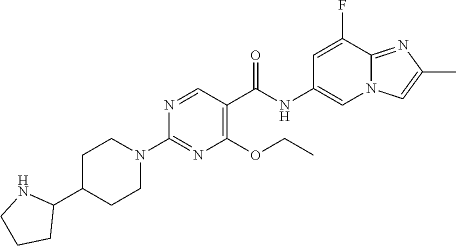 [SMILES: CCOc1nc(N2CCC(C3CCCN3)CC2)ncc1C(=O)Nc1cc(F)c2nc(C)cn2c1] |
| 204 | 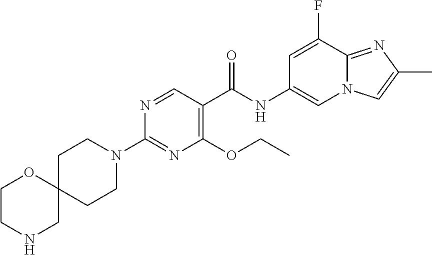 [SMILES: CCOc1nc(N2CCC3(CC2)CNCCO3)ncc1C(=O)Nc1cc(F)c2nc(C)cn2c1] |
| 205 | 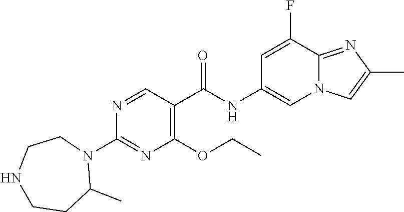 [SMILES: CCOc1nc(N2CCNCCC2C)ncc1C(=O)Nc1cc(F)c2nc(C)cn2c1] |
| 206 | 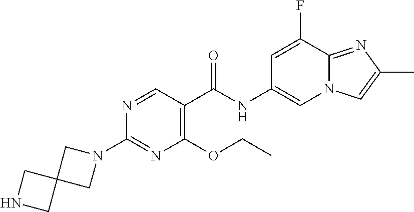 [SMILES: CCOc1nc(N2CC3(CNC3)C2)ncc1C(=O)Nc1cc(F)c2nc(C)cn2c1] |
| 207 | 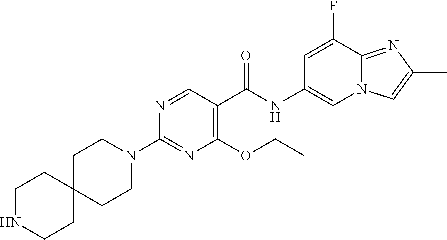 [SMILES: CCOc1nc(N2CCC3(CCNCC3)CC2)ncc1C(=O)Nc1cc(F)c2nc(C)cn2c1] |
| 208 | 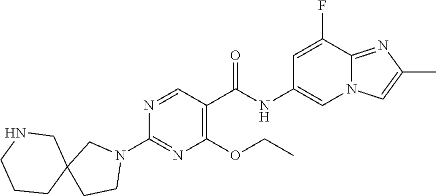 [SMILES: CCOc1nc(N2CCC3(CCCNC3)C2)ncc1C(=O)Nc1cc(F)c2nc(C)cn2c1] |
| 209 | 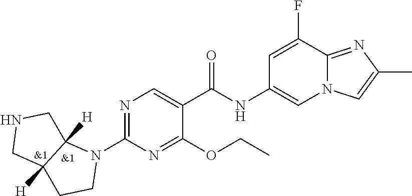 [SMILES: CCOc1nc(N2CC[C@@H]3CNC[C@@H]32)ncc1C(=O)Nc1cc(F)c2nc(C)cn2c1] |
| 210 | 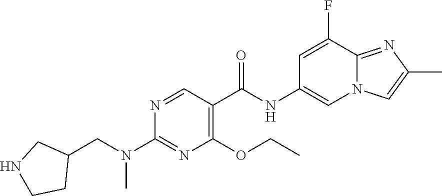 [SMILES: CCOc1nc(N(C)CC2CCNC2)ncc1C(=O)Nc1cc(F)c2nc(C)cn2c1] |
| 211 | 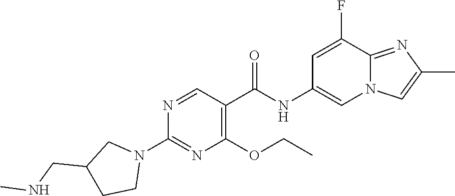 |
| 212 | 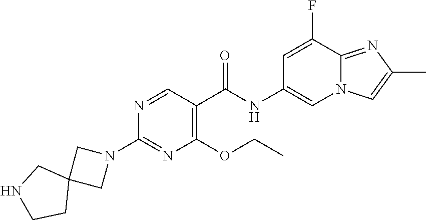 [SMILES: CCOc1nc(N2CC3(CCNC3)C2)ncc1C(=O)Nc1cc(F)c2nc(C)cn2c1] |
| 213 | 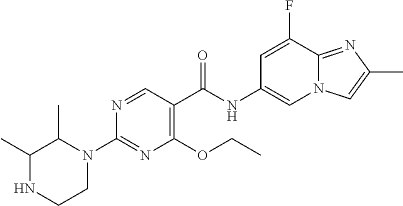 |
| 214 | 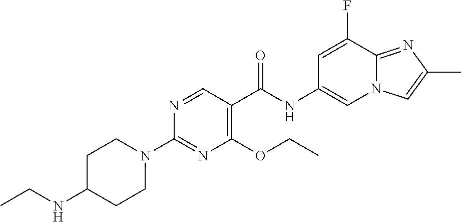 [SMILES: CCNC1CCN(c2ncc(C(=O)Nc3cc(F)c4nc(C)cn4c3)c(OCC)n2)CC1] |
| 215 | 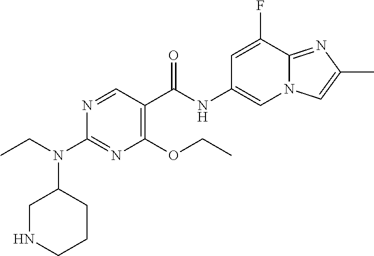 [SMILES: CCOc1nc(N(CC)C2CCCNC2)ncc1C(=O)Nc1cc(F)c2nc(C)cn2c1] |
| 216 | 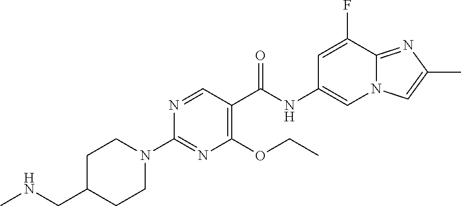 |
| 217 | 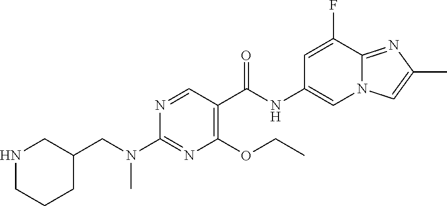 [SMILES: CCOc1nc(N(C)CC2CCCNC2)ncc1C(=O)Nc1cc(F)c2nc(C)cn2c1] |
| 218 | 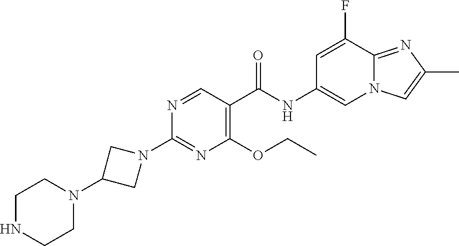 [SMILES: CCOc1nc(N2CC(N3CCNCC3)C2)ncc1C(=O)Nc1cc(F)c2nc(C)cn2c1] |
| 219 | 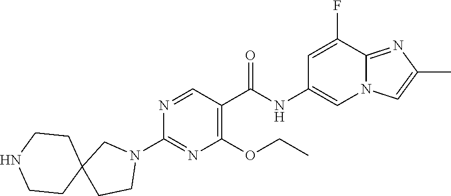 [SMILES: CCOc1nc(N2CCC3(CCNCC3)C2)ncc1C(=O)Nc1cc(F)c2nc(C)cn2c1] |
| 220 | 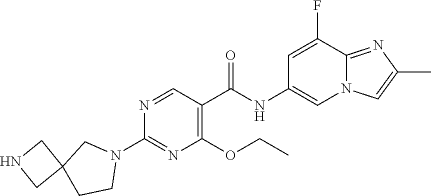 [SMILES: CCOc1nc(N2CCC3(CNC3)C2)ncc1C(=O)Nc1cc(F)c2nc(C)cn2c1] |
| 221 | 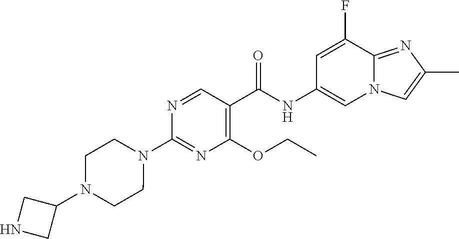 [SMILES: CCOc1nc(N2CCN(C3CNC3)CC2)ncc1C(=O)Nc1cc(F)c2nc(C)cn2c1] |
| 222 | 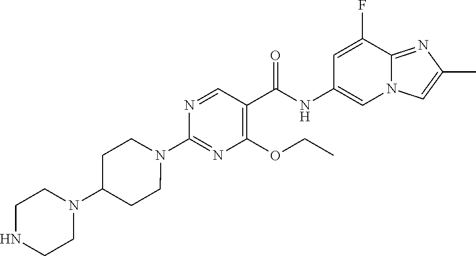 [SMILES: CCOc1nc(N2CCC(N3CCNCC3)CC2)ncc1C(=O)Nc1cc(F)c2nc(C)cn2c1] |
| 223 | 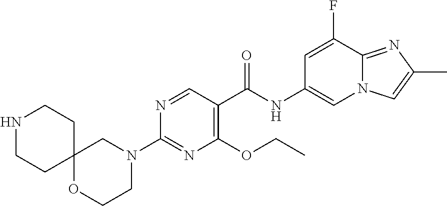 [SMILES: CCOc1nc(N2CCOC3(CCNCC3)C2)ncc1C(=O)Nc1cc(F)c2nc(C)cn2c1] |
| 224 | 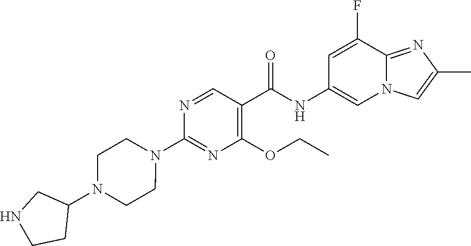 [SMILES: CCOc1nc(N2CCN(C3CCNC3)CC2)ncc1C(=O)Nc1cc(F)c2nc(C)cn2c1] |
| 225 | 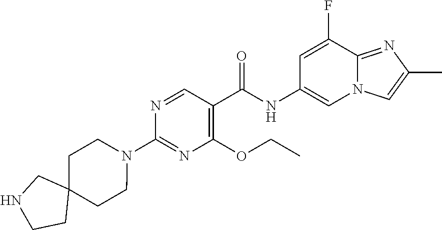 [SMILES: CCOc1nc(N2CCC3(CCNC3)CC2)ncc1C(=O)Nc1cc(F)c2nc(C)cn2c1] |
| 226 | 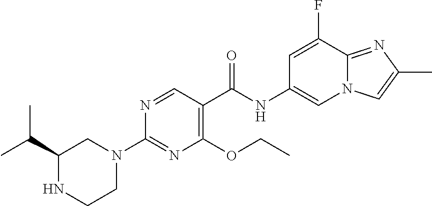 [SMILES: CCOc1nc(N2CCN[C@@H](C(C)C)C2)ncc1C(=O)Nc1cc(F)c2nc(C)cn2c1] |
| 227 | 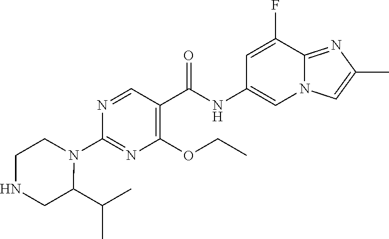 [SMILES: CCOc1nc(N2CCNCC2C(C)C)ncc1C(=O)Nc1cc(F)c2nc(C)cn2c1] |
| 228 | 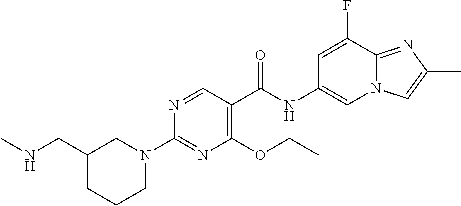 |
| 229 | 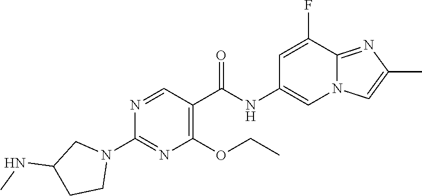 |
| 230 | 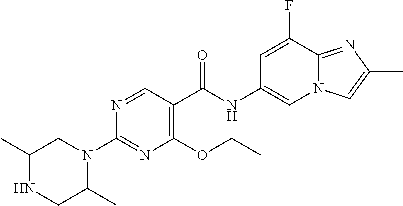 |
| 231 | 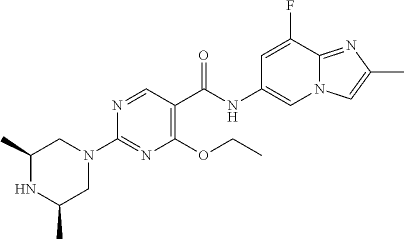 |
| 232 | 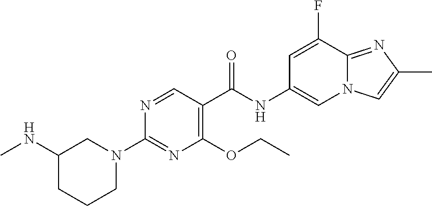 |
| 233 | 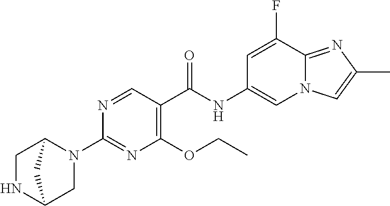 [SMILES: CCOc1nc(N2C[C@@H]3C[C@H]2CN3)ncc1C(=O)Nc1cc(F)c2nc(C)cn2c1] |
| 234 | 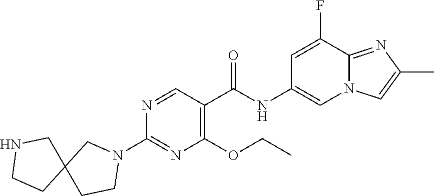 [SMILES: CCOc1nc(N2CCC3(CCNC3)C2)ncc1C(=O)Nc1cc(F)c2nc(C)cn2c1] |
| 235 | 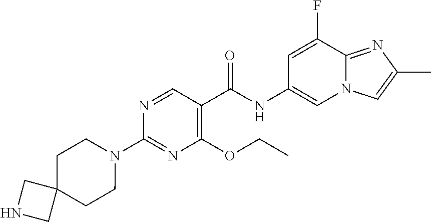 [SMILES: CCOc1nc(N2CCC3(CC2)CNC3)ncc1C(=O)Nc1cc(F)c2nc(C)cn2c1] |
| 236 | 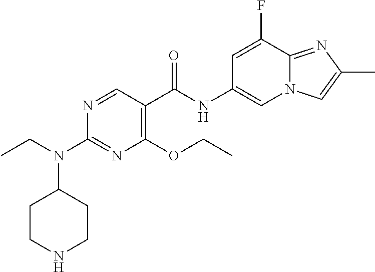 [SMILES: CCOc1nc(N(CC)C2CCNCC2)ncc1C(=O)Nc1cc(F)c2nc(C)cn2c1] |
| 237 | 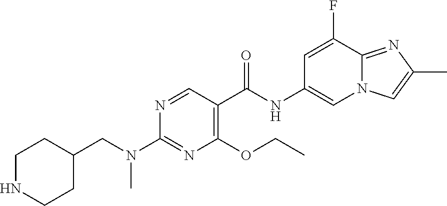 [SMILES: CCOc1nc(N(C)CC2CCNCC2)ncc1C(=O)Nc1cc(F)c2nc(C)cn2c1] |
| 238 | 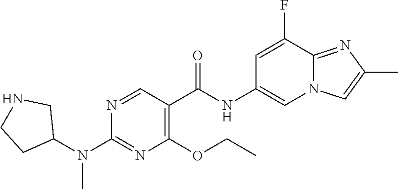 [SMILES: CCOc1nc(N(C)C2CCNC2)ncc1C(=O)Nc1cc(F)c2nc(C)cn2c1] |
| 239 | 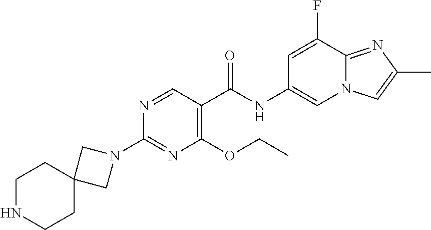 [SMILES: CCOc1nc(N2CC3(CCNCC3)C2)ncc1C(=O)Nc1cc(F)c2nc(C)cn2c1] |
| 240 | 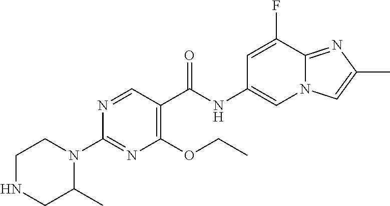 [SMILES: CCOc1nc(N2CCNCC2C)ncc1C(=O)Nc1cc(F)c2nc(C)cn2c1] |
| 241 | 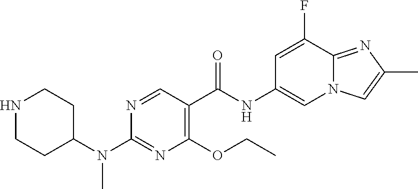 [SMILES: CCOc1nc(N(C)C2CCNCC2)ncc1C(=O)Nc1cc(F)c2nc(C)cn2c1] |
| 242 | 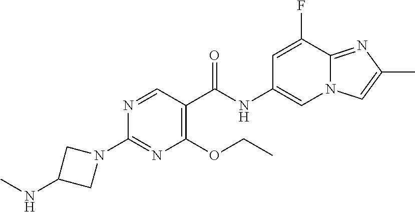 |
| 243 | 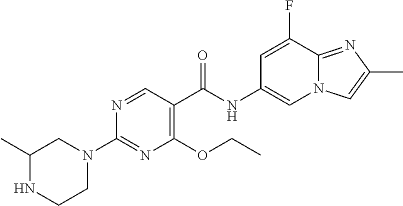 |
| 244 | 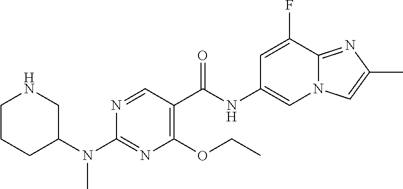 [SMILES: CCOc1nc(N(C)C2CCCNC2)ncc1C(=O)Nc1cc(F)c2nc(C)cn2c1] |
| 245 | 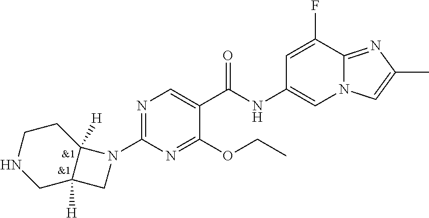 [SMILES: CCOc1nc(N2C[C@H]3CNCC[C@H]32)ncc1C(=O)Nc1cc(F)c2nc(C)cn2c1] |
| 246 | 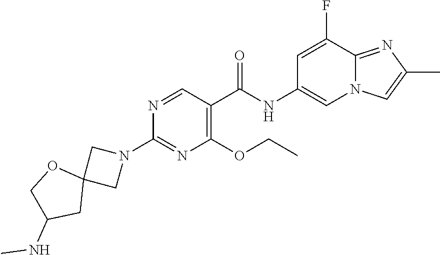 |
| 247 | 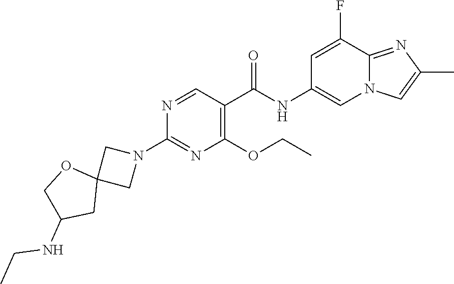 [SMILES: CCNC1COC2(C1)CN(c1ncc(C(=O)Nc3cc(F)c4nc(C)cn4c3)c(OCC)n1)C2] |
| 248 | 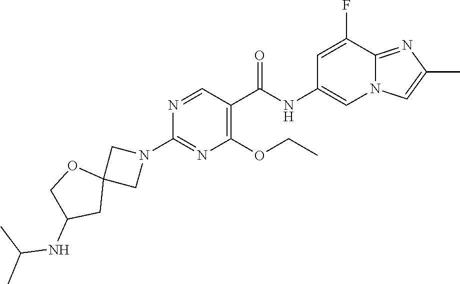 [SMILES: CCOc1nc(N2CC3(CC(NC(C)C)CO3)C2)ncc1C(=O)Nc1cc(F)c2nc(C)cn2c1] |
| 249 | 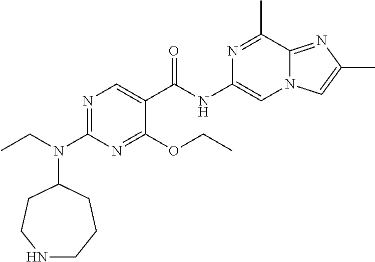 [SMILES: CCOc1nc(N(CC)C2CCCNCC2)ncc1C(=O)Nc1cn2cc(C)nc2c(C)n1] |
| 250 | 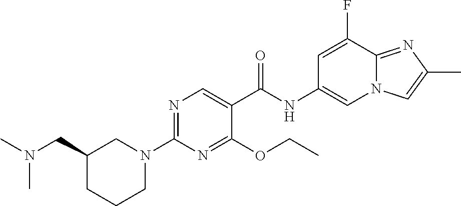 |
| 251 | 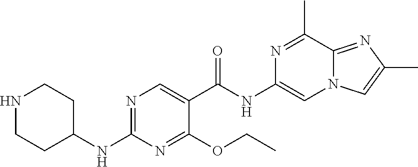 [SMILES: CCOc1nc(NC2CCNCC2)ncc1C(=O)Nc1cn2cc(C)nc2c(C)n1] |
| 252 | 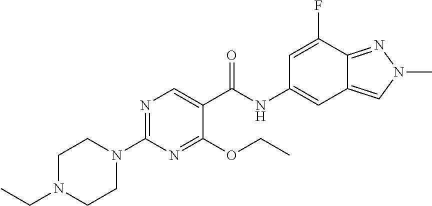 [SMILES: CCOc1nc(N2CCN(CC)CC2)ncc1C(=O)Nc1cc(F)c2nn(C)cc2c1] |
| 253 | 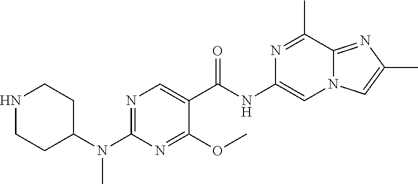 [SMILES: COc1nc(N(C)C2CCNCC2)ncc1C(=O)Nc1cn2cc(C)nc2c(C)n1] |
| 254 | 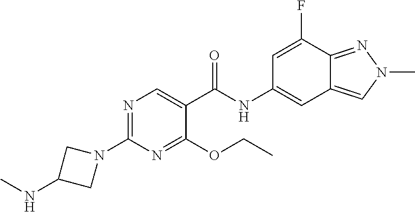 |
| 255 | 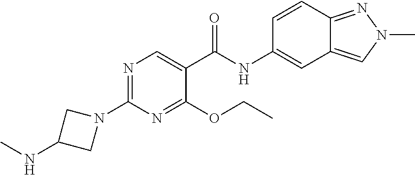 |
| 256 | 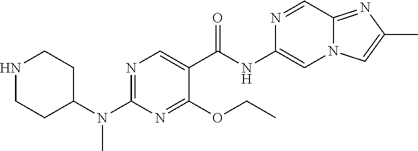 [SMILES: CCOc1nc(N(C)C2CCNCC2)ncc1C(=O)Nc1cn2cc(C)nc2cn1] |
| 257 | 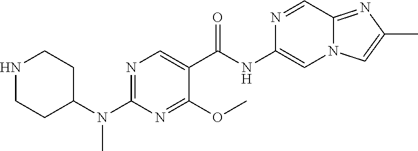 [SMILES: COc1nc(N(C)C2CCNCC2)ncc1C(=O)Nc1cn2cc(C)nc2cn1] |
| 258 | 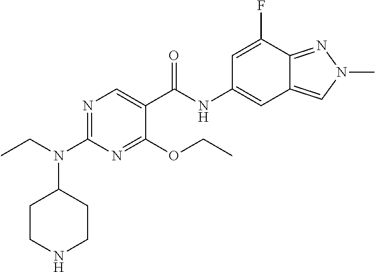 [SMILES: CCOc1nc(N(CC)C2CCNCC2)ncc1C(=O)Nc1cc(F)c2nn(C)cc2c1] |
| 259 | 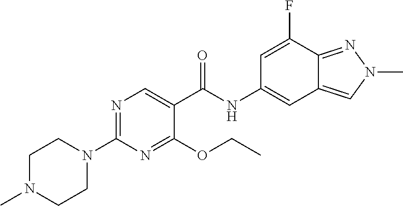 |
| 260 | 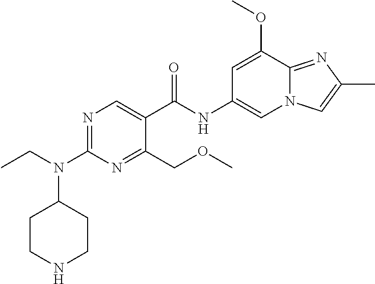 [SMILES: CCN(c1ncc(C(=O)Nc2cc(OC)c3nc(C)cn3c2)c(COC)n1)C1CCNCC1] |
| 261 | 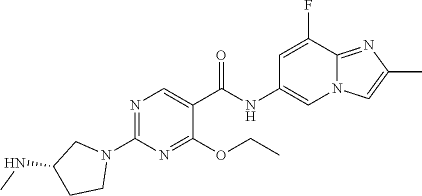 |
| 262 | 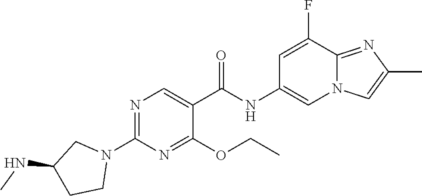 |
| 263 | 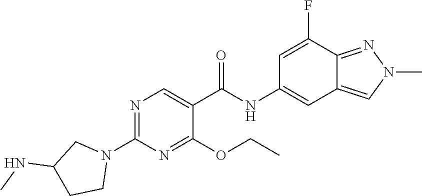 |
| 264 | 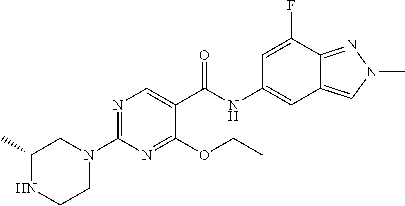 |
| 265 | 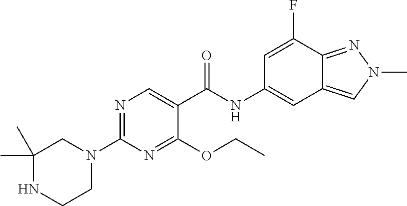 |
| 266 | 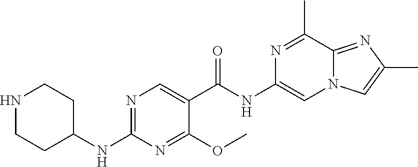 [SMILES: COc1nc(NC2CCNCC2)ncc1C(=O)Nc1cn2cc(C)nc2c(C)n1] |
| 267 | 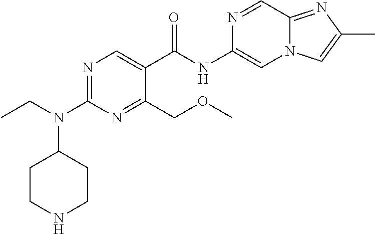 [SMILES: CCN(c1ncc(C(=O)Nc2cn3cc(C)nc3cn2)c(COC)n1)C1CCNCC1] |
| 268 | 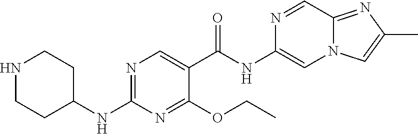 [SMILES: CCOc1nc(NC2CCNCC2)ncc1C(=O)Nc1cn2cc(C)nc2cn1] |
| 269 | 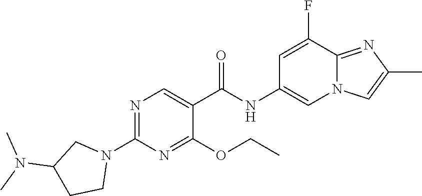 |
| 270 | 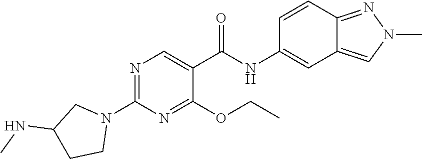 |
| 271 | 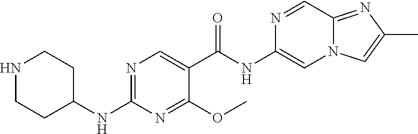 [SMILES: COc1nc(NC2CCNCC2)ncc1C(=O)Nc1cn2cc(C)nc2cn1] |
| 272 | 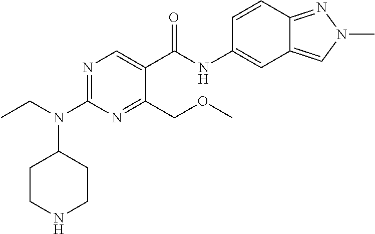 [SMILES: CCN(c1ncc(C(=O)Nc2ccc3nn(C)cc3c2)c(COC)n1)C1CCNCC1] |
| 273 | 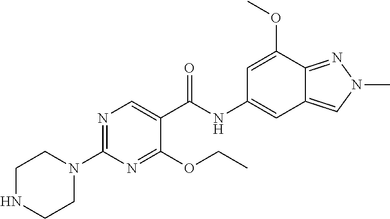 [SMILES: CCOc1nc(N2CCNCC2)ncc1C(=O)Nc1cc(OC)c2nn(C)cc2c1] |
| 274 | 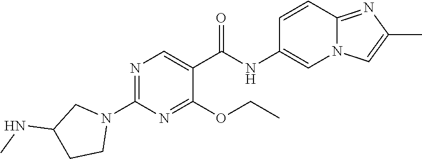 |
| 275 | 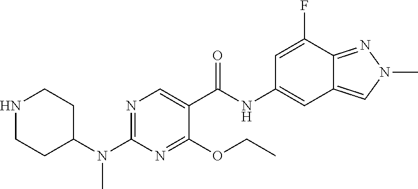 [SMILES: CCOc1nc(N(C)C2CCNCC2)ncc1C(=O)Nc1cc(F)c2nn(C)cc2c1] |
| 276 | 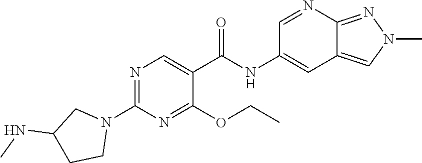 |
| 277 | 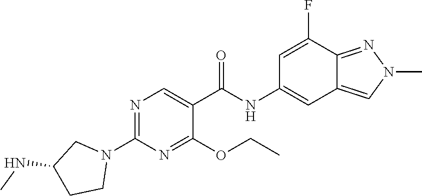 |
| 278 | 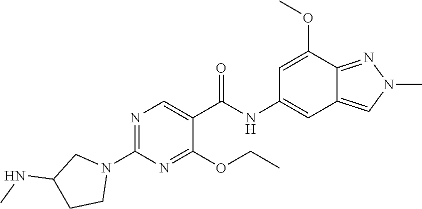 |
| 279 | 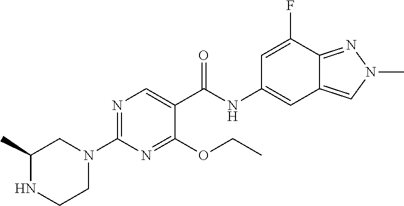 |
| 280 | 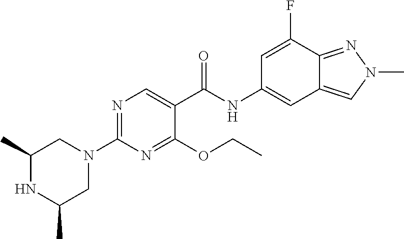 |
| 281 | 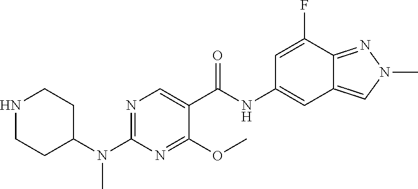 [SMILES: COc1nc(N(C)C2CCNCC2)ncc1C(=O)Nc1cc(F)c2nn(C)cc2c1] |
| 282 | 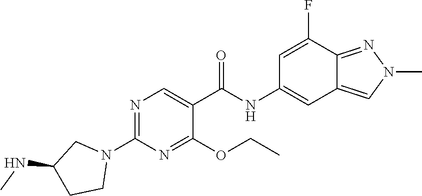 |
| 283 | 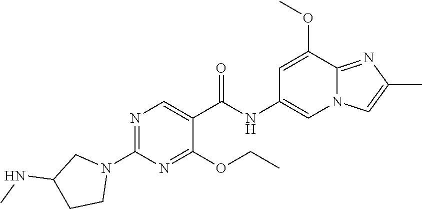 |
| 284 | 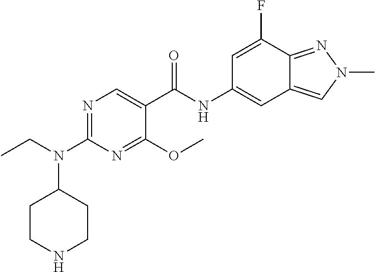 [SMILES: CCN(c1ncc(C(=O)Nc2cc(F)c3nn(C)cc3c2)c(OC)n1)C1CCNCC1] |
| 285 | 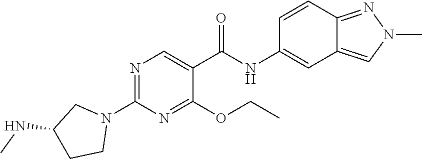 |
| 286 | 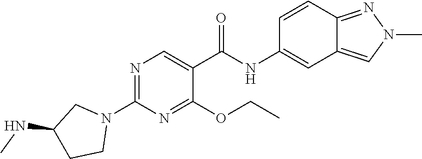 |
| 287 | 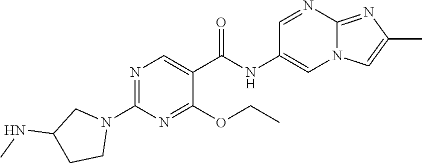 |
| 288 | 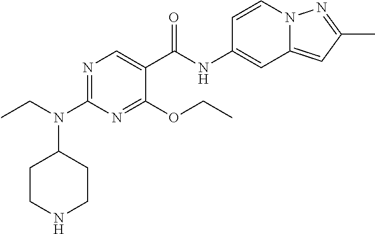 [SMILES: CCOc1nc(N(CC)C2CCNCC2)ncc1C(=O)Nc1ccn2nc(C)cc2c1] |
| 289 | 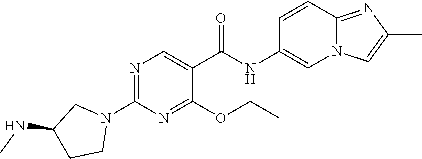 |
| 290 | 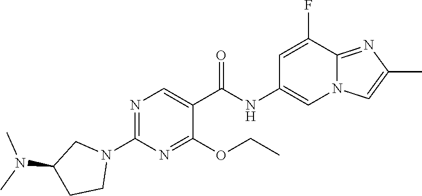 |
| 291 | 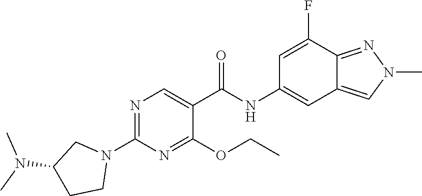 |
| 292 | 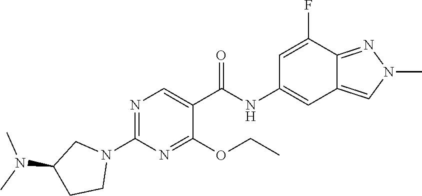 |
| 293 | 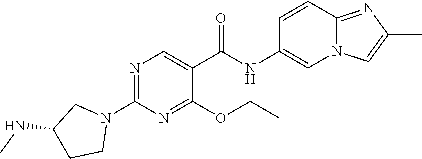 |
| 294 | 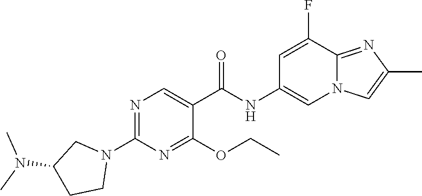 |
| 295 | 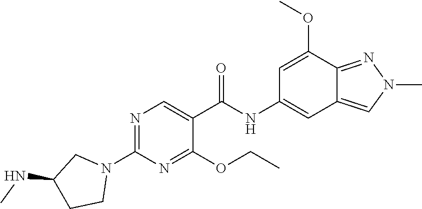 |
| 296 | 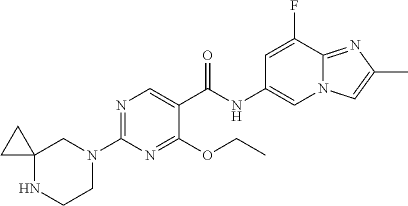 [SMILES: CCOc1nc(N2CCNC3(CC3)C2)ncc1C(=O)Nc1cc(F)c2nc(C)cn2c1] |
| 297 | 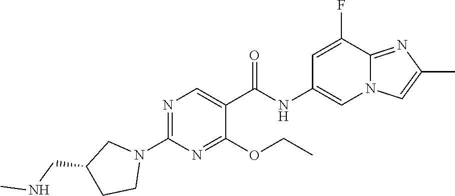 |
| 298 | 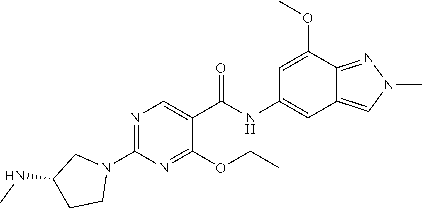 |
| 299 | 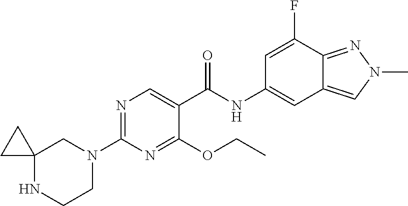 [SMILES: CCOc1nc(N2CCNC3(CC3)C2)ncc1C(=O)Nc1cc(F)c2nn(C)cc2c1] |
| 300 | 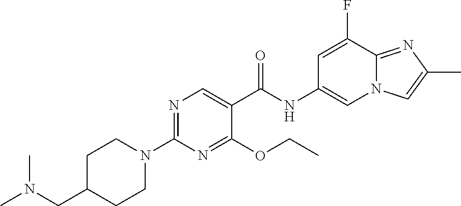 |
| 301 | 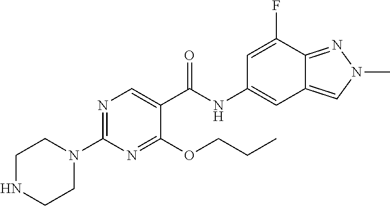 [SMILES: CCCOc1nc(N2CCNCC2)ncc1C(=O)Nc1cc(F)c2nn(C)cc2c1] |
| 302 | 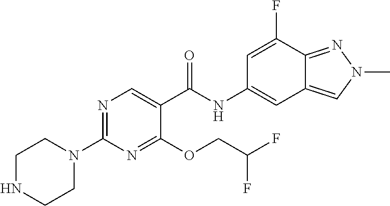 [SMILES: Cn1cc2cc(NC(=O)c3cnc(N4CCNCC4)nc3OCC(F)F)cc(F)c2n1] |
| 303 | 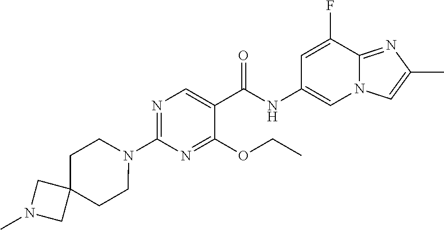 |
| 304 | 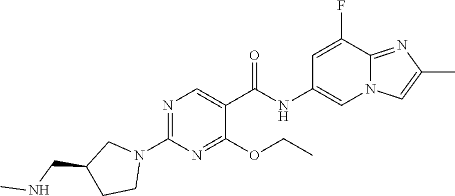 |
| 305 | 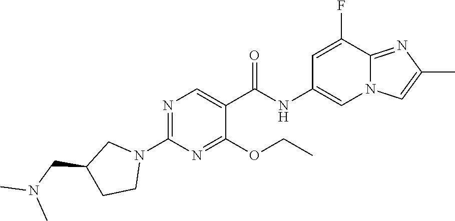 |
| 306 | 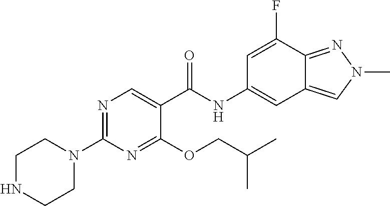 [SMILES: CC(C)COc1nc(N2CCNCC2)ncc1C(=O)Nc1cc(F)c2nn(C)cc2c1] |
| 307 | 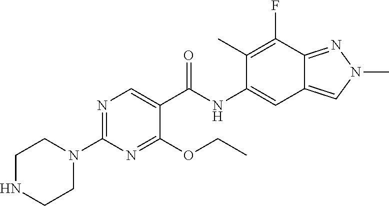 [SMILES: CCOc1nc(N2CCNCC2)ncc1C(=O)Nc1cc2cn(C)nc2c(F)c1C] |
| 308 | 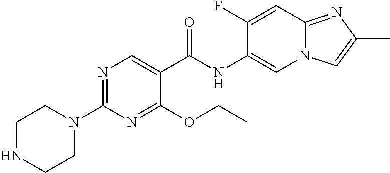 [SMILES: CCOc1nc(N2CCNCC2)ncc1C(=O)Nc1cn2cc(C)nc2cc1F] |
| 309 | 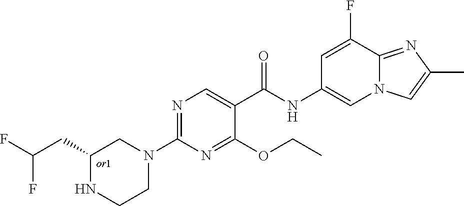 [SMILES: CCOc1nc(N2CCN[C@H](CC(F)F)C2)ncc1C(=O)Nc1cc(F)c2nc(C)cn2c1] |
| 310 | 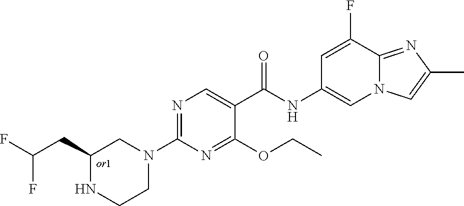 [SMILES: CCOc1nc(N2CCN[C@@H](CC(F)F)C2)ncc1C(=O)Nc1cc(F)c2nc(C)cn2c1] |
| 311 | 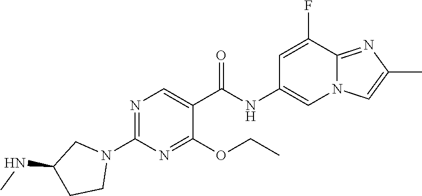 |
| 312 | 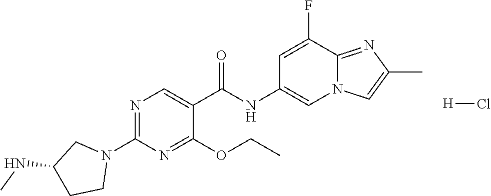 |
| 313 | 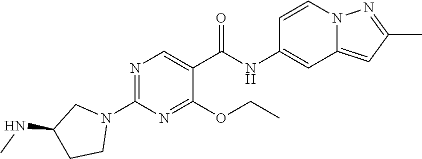 |
| 314 | 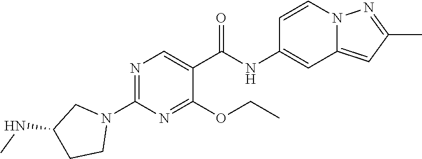 |
| 315 | 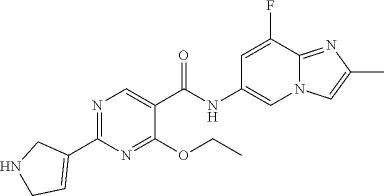 [SMILES: CCOc1nc(C2=CCNC2)ncc1C(=O)Nc1cc(F)c2nc(C)cn2c1] |
| 316 | 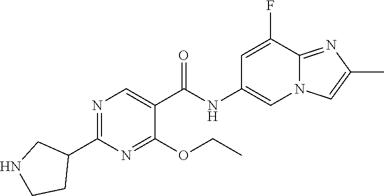 [SMILES: CCOc1nc(C2CCNC2)ncc1C(=O)Nc1cc(F)c2nc(C)cn2c1] |
| 317 | 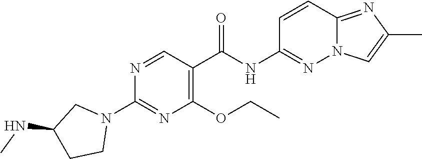 |
| 318 | 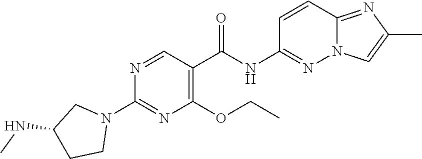 |
| 319 | 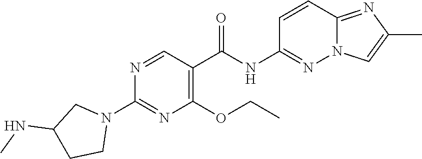 |

or a pharmaceutically acceptable salt, solvate, or prodrug thereof.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

It will be understood that the compounds of any one of the Formulae disclosed herein and any pharmaceutically acceptable salts thereof, comprise stereoisomers and mixtures of stereoisomers of all isomeric forms of said compounds.

It is to be understood that the compounds of any Formula described herein include the compounds themselves, as well as their salts, and their solvates, if applicable.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

By way of example only, provided is a scheme for preparing the small molecule splicing modulators (SMSMs) described herein.

In some embodiments, a scheme for preparing an SMSM is described herein in

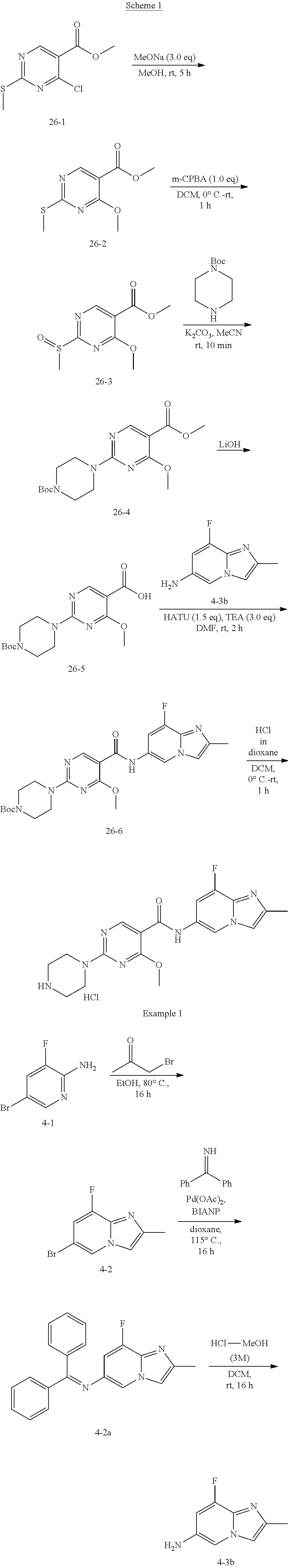

In some aspects, the present disclosure provides a method of preparing a compound of the present disclosure.

In some aspects, the present disclosure provides a method of preparing a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) has been synthesised by any one of the processes defined herein, the processes may then further comprise the additional steps of: (i) removing any protecting groups present; (ii) converting the compound of Formula (I) into another compound of Formula (I), (iii) forming a pharmaceutically acceptable salt, hydrate or solvate thereof; and/or (iv) forming a prodrug thereof.

The resultant compounds of Formula (I) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulphoxides, such as dimethyl sulphoxide (DMSO): nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds. As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply-whenever necessary or useful-synthetic protecting (or protective) groups; suitable protecting groups as well as methods for introducing and removing them are well-known to the person skilled in the art of chemical synthesis and are described, in more detail, in, e.g., P.G.M. Wuts, T.W. Greene, “Greene's Protective Groups in Organic Synthesis”, 4th edition (2006) (John Wiley & Sons).

General routes for the preparation of a compound of the application are described herein.

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulphated β-cyclodextrin (S-β-CD), maltosyl-β-cyclodextrin, β-cyclodextrin sulphobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilise the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ¿-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier. In some embodiments, provided herein is a pharmaceutical composition comprising a compound of the disclosure as defined hereinbefore, or a pharmaceutically acceptable salt, solvate, or prodrug thereof and one or more pharmaceutically acceptable excipients.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present disclosure for use in therapy is an amount sufficient to treat or prevent a disease or disorder referred to herein, slow its progression and/or reduce the symptoms associated with the condition.

An effective amount of a compound of the present disclosure for use in therapy is an amount sufficient to treat a disease or disorder referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or subject and the route of administration, according to well-known principles of medicine.

### Methods of Use

Provided herein is a method of treating a disorder related to a nucleotide repeat expansion, comprising administering to the subject a therapeutically effective amount of a compound or a pharmaceutical composition described herein.

In some embodiments, the nucleotide repeat expansion comprises a nucleotide sequence repeated two or more times, wherein the nucleotide sequence is selected from the group consisting of CAG, CAG/CTG, GCG, GCN, CGG, CCG, CCCCGCCCCGCG, GCA, GGGGCC, CTG, GAA, ATTCT, TGGAA, GGCCTG, AAGGG, CCCTCT, ATTTT/ATTTC, and CCCTCT.

In some embodiments, the nucleotide repeat expansion comprises a trinucleotide sequence repeated two or more times, wherein the trinucleotide sequence is selected from the group consisting of CAG, CTG, CGG, and GCN.

Provided herein is a method of treating a disease in a subject in need thereof, the method comprising administering to the subject a therapeutically effective amount of a compound or a pharmaceutical composition described herein, wherein the disease is selected from the group consisting of Dentatorubropallidoluysian atrophy. Huntington's disease, Spinal and bulbar muscular atrophy, SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), SCA12 (Spinocerebellar ataxia Type 12), SCA17 (Spinocerebellar ataxia Type 17), FRAXA (Fragile X syndrome), FXTAS (Fragile X-associated tremor/ataxia syndrome), FRAXE (Fragile XE mental retardation), Baratela-Scott syndrome, FRDA (Friedreich's ataxia), DM1 (Myotonic dystrophy Type 1), DM2 (Myotonic dystrophy Type 2) SCA8 (Spinocerebellar ataxia Type 8), Fuchs endothelial comeal dystrophy, Desbuquois dysplasia, amyotrophic lateral sclerosis, and frontotemporal dementia

In some embodiments, the disease is Huntington's disease.

Provided herein is a method of treating a disease in a subject in need thereof, the method comprising administering to the subject a therapeutically effective amount of a compound or a pharmaceutical composition disclosed herein, wherein the disease is Huntington's disease.

Provided herein is a method of treating a disease in a subject in need thereof, the method comprising administering to the subject a therapeutically effective amount of a compound or a pharmaceutical composition disclosed herein, wherein the disease is Myotonic ystrophy.

In some embodiments, the disease is FRAXA (Fragile X syndrome), FXTAS (Fragile X-associated tremor/ataxia syndrome), or FRAXE (Fragile XE mental retardation).

Provided herein is a method of treating a disease in a subject in need thereof, the method comprising administering to the subject a therapeutically effective amount of a compound or a pharmaceutical composition disclosed herein, wherein the disease is FRAXA (Fragile X syndrome), FXTAS (Fragile X-associated tremor/ataxia syndrome), or FRAXE (Fragile XE mental retardation).

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

The present disclosure includes the following embodiments 1-35:

1. A compound of Formula (I):

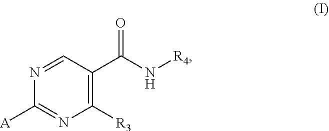

or a pharmaceutically acceptable salt, solvate, or prodrug thereof, wherein:
A is saturated or partially unsaturated mono-bi- or tri-cyclic 4- to 14-membered heterocycloalkyl or NR: R₂, wherein the heterocycloalkyl comprises 1 or 2 nitrogen ring atoms and is optionally substituted with 1, 2, 3, or 4 R₆;
R₁ is —(CH₂)₀₋₂-heterocycloalkyl of 4-14 ring atoms comprising at least one nitrogen ring atom and 0-2 additional ring heteroatoms independently selected from N, O, and S; R₁ optionally substituted with 1, 2, 3, or 4 R₆;
R₂ is hydrogen, C₁₋₇alkyl, or C₃₋₈cycloalkyl;
R₃ is H, halo, C₁₋₇alkyl, OR₅, N(R₅)₂, C₃₋₈cycloalkyl, or heterocycloalkyl;
R₄ is aryl, or bicyclic 9-membered heteroaryl comprising 2, 3, or 4 heteroatoms independently selected from N, O, and S, wherein R₄ is optionally substituted with 1, 2, or 3 R₇;
each R₅ is independently C₁₋₇alkyl, C₃₋₈cycloalkyl, or heterocycloalkyl;
each R₆ is independently hydroxy, C₁₋₇alkyl, amino, C₁₋₇heteroalkyl, —(CH₂)₀₋₃—C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen or OH, and cycloalkyl and heterocycloalkyl are optionally substituted with 1-4 independently selected C₁₋₇alkyl, halogen, or OH;
   or two R₆ together form oxo (═O);
   or two R₆ together form C₁₋₇alkylene to form a ring;
   each R′ is hydrogen or C₁₋₇alkyl; and
each R; is independently halo, cyano, C₁₋₇alkyl, C₁₋₇haloalkyl, C₁₋₇alkoxy, C₁₋₇haloalkoxy, or C₃₋₈cycloalkyl, wherein the C₁₋₇alkyl is optionally substituted with OH.

2. A compound of Formula (I):

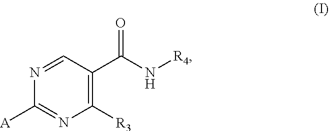

or a pharmaceutically acceptable salt, solvate, or prodrug thereof, wherein:
A is saturated or partially unsaturated mono- or bi-cyclic 4- to 11-membered nitrogen-containing heterocycloalkyl or NR: R₂, wherein the nitrogen-containing heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₂ is hydrogen, C₁₋₇alkyl, or C₃₋₈cycloalkyl;
R₃ is H, C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O, wherein R₄ can be optionally substituted with 1, 2, or 3 R₇;
each R₅ is independently C₁₋₇alkyl, C₃₋₈cycloalkyl, or heterocycloalkyl;
each R₆ is independently hydroxy, C₁₋₇alkyl, amino, C₁₋₇heteroalkyl, —(CH₂)₀₋₃—C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen or OH, and cycloalkyl and heterocycloalkyl are optionally substituted with 1-4 independently selected C₁₋₇alkyl, halogen, or OH;
   or two R₆ together form oxo (═O);
   or two R₆ together form C₁₋₇alkylene to form a ring; and
each R₇ is independently halo, C₁₋₇alkyl, C₁₋₇haloalkyl, C₁₋₈alkoxy, or C₁₋₇haloalkoxy.

3. A compound according to any of the preceding embodiments, wherein:
A is 4-11 membered nitrogen-containing heterocycloalkyl, wherein the nitrogen-containing heterocycloalkyl comprises 1 or 2 nitrogen ring atoms and 0-2 additional ring heteroatoms selected from O and S, and is optionally substituted with 1, 2, 3, or 4 R₆; and
each R₆ is independently C₁₋₇alkyl, amino, C₁₋₇alkylamino, C₃₋₈cycloalkyl, heterocycloalkyl and C₁₋₇alkoxy-heterocycloalkyl or two R₆ together form C₁₋₇alkylene.

4. A compound according to any one of the preceding embodiments, wherein each R₆ is independently C₁₋₇ alkyl, heterocycloalkyl, or C₁₋₇alkoxy-heterocycloalkyl or two R₆ together form C₁₋₇alkylene.

5. A compound according to any one of the preceding embodiments, wherein each R₆ is independently methyl, ethyl, isopropyl, methoxy-azetidinyl, or pyrrolidinyl, or two R₆ together form ethylene or propylene.

6. A compound according to any one of the preceding embodiments, wherein
A is

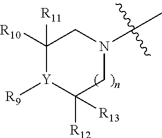

Y is absent, N or CH;
R₉ is hydrogen, C₁₋₇alkyl, or (CH₂)ₘ—NR₁₄R₁₅;
R₁₀ is hydrogen or C₁₋₇alkyl;
R₁₁ is hydrogen or C₁₋₇alkyl;
R₁ z is hydrogen or C₁₋₇alkyl;
R₁₃ is hydrogen or C₁₋₇alkyl;
each R₁₄ and R₁₅ are independently hydrogen, C₁₋₇alkyl and C₃-cycloalkyl;
n is 0, 1 or 2;
m is 0, 1 or 2;
or R₉ and R₁₀ together form C₁₋₇alkylene;
or R₉ and R₁₂ together form C₁₋₇alkylene;
or R₁₀ and R₁₁ together form C₂₋₇alkylene or 4- to 6-membered heterocycloalkyl optionally substituted with C₁₋₇alkyl;
or R₁₀ and R₁₂ together form C₁₋₇alkylene or 4- to 6-membered heterocycloalkyl optionally substituted with C₁₋₇alkyl;
or R₁₀ and R₁₄ together form C₁₋₇alkylene;
or R₁₂ and R₁₅ together form C₂₋₇alkylene;
or R₁₂ and Ria together form C₁₋₇alkylene;
or R₁₄ and R₁₅ together form C₂₋₇alkylene which is optionally substituted with C₁₋₇alkoxy.

7. A compound according to any one of the preceding embodiments, wherein Y is N.

8. A compound according to any one of the preceding embodiments, wherein Y is CH and Ry is (CH₂)ₘ—NR₁₄R₁₅.

9. A compound according to any one of the preceding embodiments, wherein n is 1.

10. A compound according to any one of the preceding embodiments, wherein R₉ is hydrogen, pyrrolidinyl, or methoxy-azetidinyl.

11. A compound according to any one of the preceding embodiments, wherein R₁₀ is hydrogen, methyl, ethyl or isopropyl.

12. A compound according to any one of the preceding embodiments, wherein R₁₁ is hydrogen or methyl.

13. A compound according to any one of the preceding embodiments, wherein R₁₂ is hydrogen or methyl.

14. A compound according to any one of the preceding embodiments, wherein R₁₃ is hydrogen.

15. A compound according to any one of the preceding embodiments, wherein R₉ and R₁₀ together form propylene.

16. A compound according to any one of the preceding embodiments, wherein R₁₀ and Rn together form ethylene.

17. A compound according to any one of the preceding embodiments, wherein R₁₄ and R₁₅ together form propylene or butylene.

18. A compound according to any one of the preceding embodiments, wherein A is

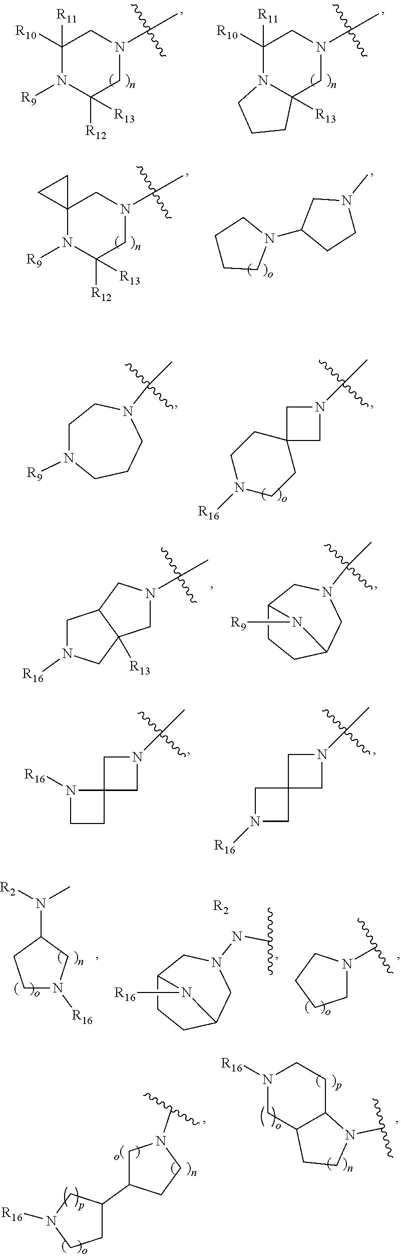

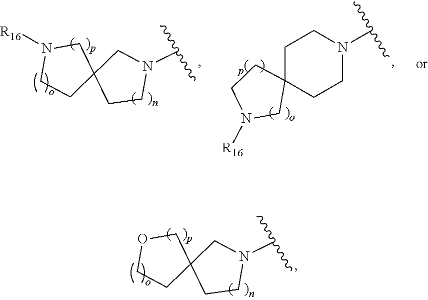

wherein R₉, R₁₀, R₁₁, R₁₂, and R₁₃ are as defined in any one of the preceding embodiments; each R₁₆ is independently hydrogen or C₁₋₇alkyl; each p is 0, 1, or 2; each o is 0, 1, or 2; and each A is optionally substituted with one or two R₆.

19. A compound according to any one of the preceding embodiments, wherein A is piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl, wherein piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl are each optionally substituted with 1, 2, 3, or 4 R₆.

20. A compound according to any one of the preceding embodiments, wherein A is NR₁R₂.

21. A compound according to any one of the preceding embodiments, wherein A is

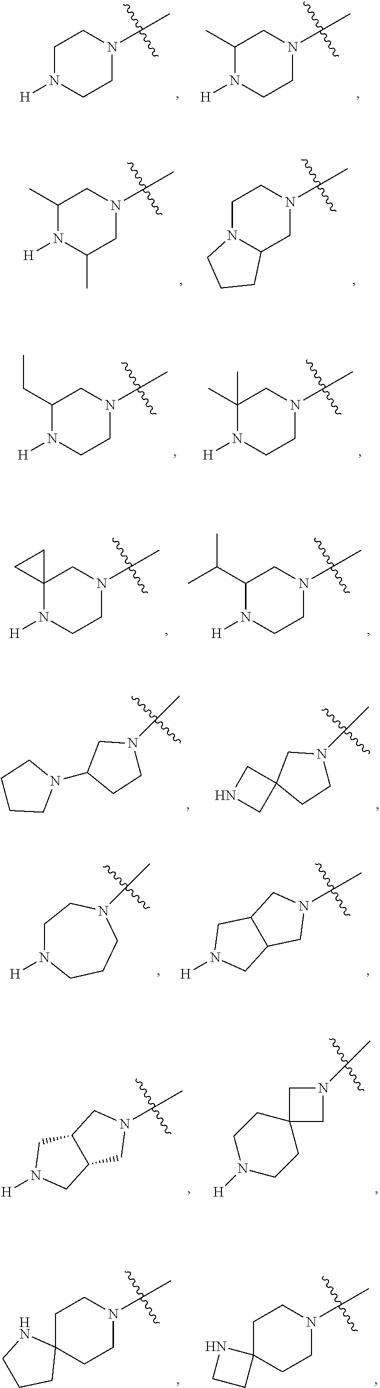

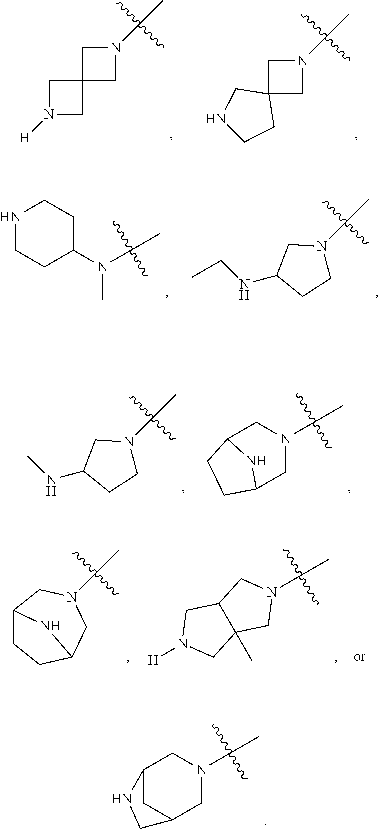

22. A compound according to any one of the preceding embodiments, wherein Rₐ is a bicyclic 9-membered heteroaryl comprising 2 heteroatoms independently selected from N and O, wherein Rₐ can be optionally substituted with 1, 2, or 3 R₇.

23. A compound according to any one of the preceding embodiments, wherein Rₐ is a bicyclic 9-membered heteroaryl comprising 2 heteroatoms independently selected from N and O, wherein R₄ is substituted with 1 or 2 R₇.

24. A compound according to any one of the preceding embodiments, wherein Rₐ is

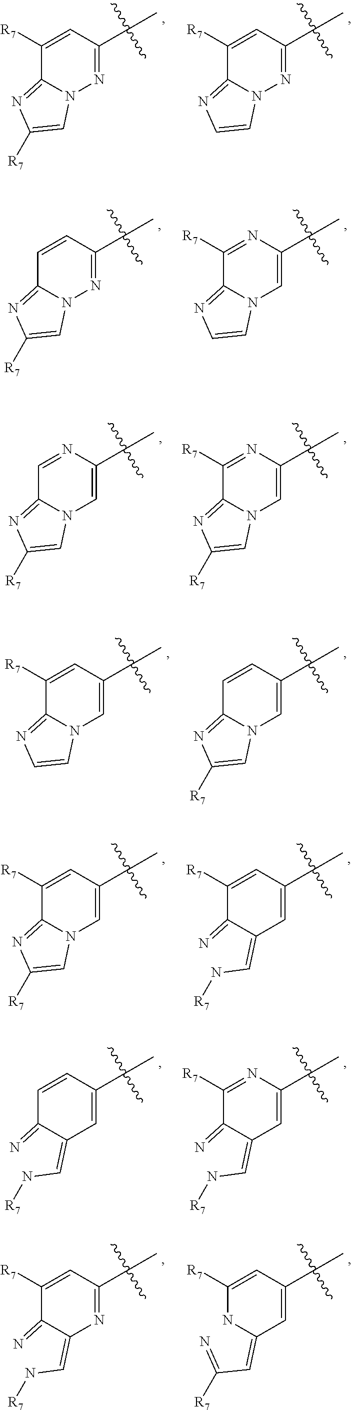

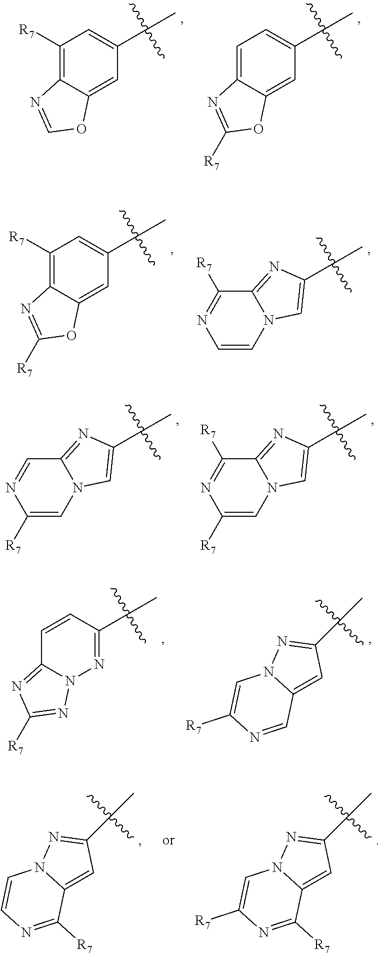

25. A compound according to any one of the preceding embodiments, wherein Rₐ is

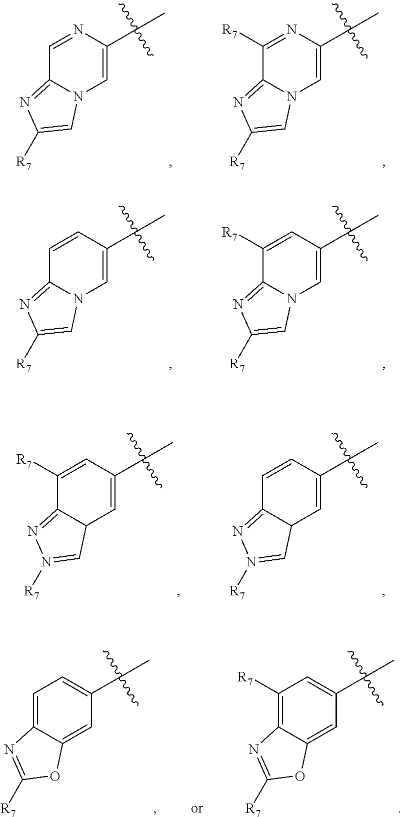

26. A compound according to any one of the preceding embodiments, wherein Rₐ is selected from imidazo[1,2-a]pyrazine, benzo[d]oxazole, or imidazo[1,2-a]pyrazine, wherein imidazo[1,2-a]pyrazine, benzo[d]oxazole, or imidazo[1,2-a]pyrazine are each optionally substituted with 1, 2, 3 or 4 R₇.

27. A compound according to any one of the preceding embodiments, wherein Rₐ is

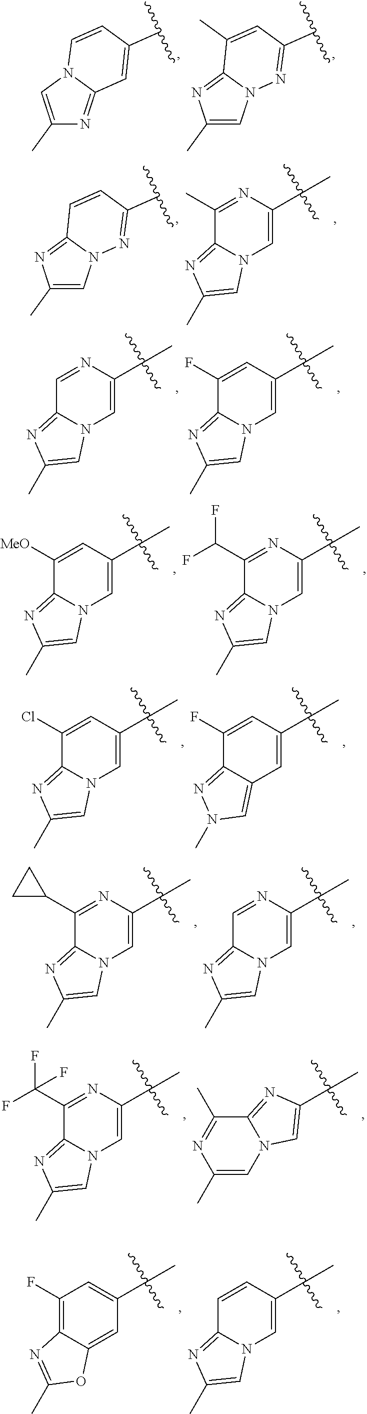

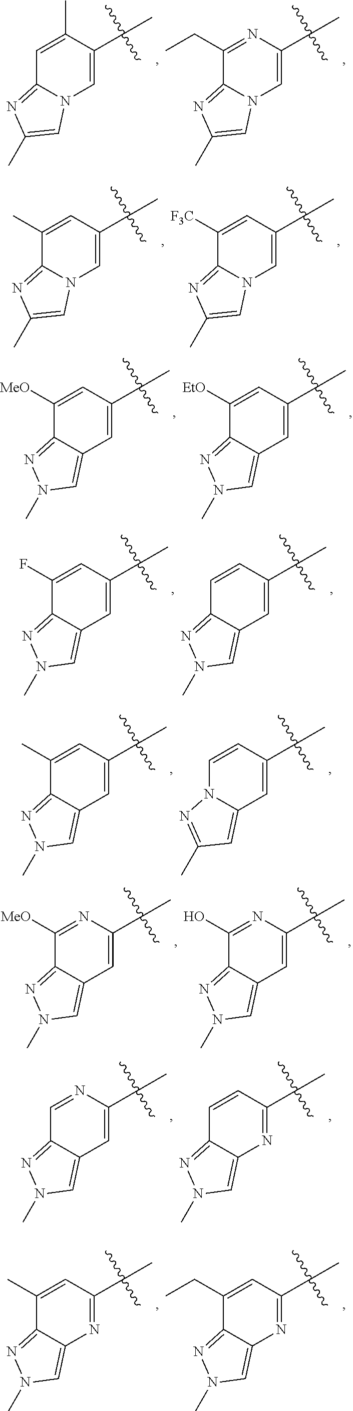

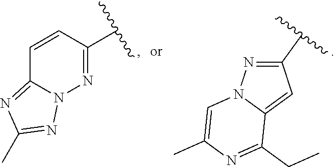 [SMILES: *c1ccc2nc(C)nn2n1 ; *c1cc2c(CC)nc(C)cn2n1]

28. A compound according to any one of the preceding embodiments, wherein R₄ is

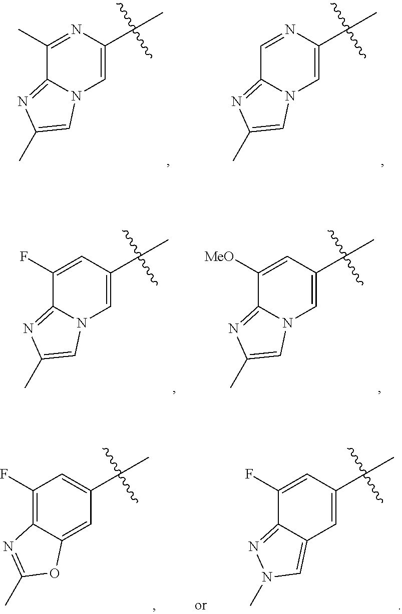 [SMILES: *c1cn2cc(C)nc2c(C)n1 ; *c1cn2cc(C)nc2cn1 ; *c1cc(F)c2nc(C)cn2c1 ; *c1cc(OC)c2nc(C)cn2c1 ; *c1cc(F)c2nc(C)oc2c1 ; *c1cc(F)c2nn(C)cc2c1]

29. A compound according to any one of the preceding embodiments, selected from a compound of Table 1.

30. A compound according to any one of the preceding embodiments, or a pharmaceutically acceptable salt, solvate, or prodrug thereof for use as a small molecule splicing modulator.

31. A pharmaceutical composition comprising a compound according to any one of the preceding embodiments, or a pharmaceutically acceptable salt, solvate, or prodrug thereof and one or more pharmaceutically acceptable excipients.

32. A method of treating a disease in a subject in need thereof, the method comprising administering to the subject a therapeutically effective amount of a compound of any one of embodiments 1-30 or a pharmaceutical composition of embodiment 31, wherein the disease is selected from the group consisting of Dentatorubropallidoluysian atrophy, Huntington's disease, Spinal and bulbar muscular atrophy, SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6). SCA7 (Spinocerebellar ataxia Type 7). SCA12 (Spinocerebellar ataxia Type 12), SCA17 (Spinocerebellar ataxia Type 17), FRAXA (Fragile X syndrome), FXTAS (Fragile X-associated tremor/ataxia syndrome), FRAXE (Fragile XE mental retardation), Baratela-Scott syndrome, FRDA (Friedreich's ataxia), DM1 (Myotonic dystrophy Type 1), DM2 (Myotonic dystrophy Type 2) SCA8 (Spinocerebellar ataxia Type 8), Fuchs endothelial corneal dystrophy, Desbuquois dysplasia, amyotrophic lateral sclerosis, frontotemporal dementia

33. A method of treating a disease in a subject in need thereof, the method comprising administering to the subject a therapeutically effective amount of a compound of any one of embodiments 1-30 or a pharmaceutical composition of embodiment 31, wherein the disease is Huntington's disease.

34. A method of treating a disease in a subject in need thereof, the method comprising administering to the subject a therapeutically effective amount of a compound of any one of embodiments 1-30 or a pharmaceutical composition of embodiment 31, wherein the disease is Myotonic dystrophy 1.

35. A method of treating a disease in a subject in need thereof, the method comprising administering to the subject a therapeutically effective amount of a compound of any one of embodiments 1-30 or a pharmaceutical composition of embodiment 31, wherein the disease is FRAXA (Fragile X syndrome), FXTAS (Fragile X-associated tremor/ataxia syndrome), or FRAXE (Fragile XE mental retardation).

### EXAMPLES

For exemplary purpose, neutral compounds of Formula (I) are synthesized and tested in the examples. It is understood that the neutral compounds of Formula (I) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

Nuclear magnetic resonance (NMR) spectra were recorded at 400 MHz or 300 MHz as stated; the chemical shifts (8) are reported in parts per million (ppm). Spectra were recorded using a Bruker or Varian instrument with 8, 16 or 32 scans.

LC-MS chromatograms and spectra were recorded using an Agilent 1200 or Shimadzu LC-20 AD&MS 2020 instrument using a C-18 column such as C18 2.1×30 mm, unless otherwise stated. Injection volumes were 0.7-8.0 μl and the flow rates were typically 0.8 or 1.2 ml/min. Detection methods were diode array (DAD) or evaporative light scattering (ELSD) as well as positive ion electrospray ionisation. MS range was 100-1000 Da. Solvents were gradients of water and acetonitrile both containing a modifier (typically 0.01-0.04%) such as trifluoroacetic acid or ammonium carbonate.

### ABBREVIATIONS

ACN Acetonitrile
BINAP 2,2′-bis(diphenylphosphino)-1,1′-binaphthyl
CDCl₃ Chloroform-d
DCM Dichloromethane
DMF N,N-dimethylformamide
DMSO dimethylsulphoxide
DMSO-d₆ Hexadeuterodimethylsulphoxide
DP Desired product
eq. Equivalents
ESI Electrospray ionisation
EA ethyl acetate
FCC flash column chromatography
h hour(s)
¹H NMR Proton nuclear magnetic resonance spectroscopy
HPLC high performance liquid chromatography
LC-MS Liquid chromatography-mass spectrometry
MeOD Methanol-d₄
MeOH Methanol
min minute(s)
NaOAc Sodium acetate
NMP N-Methyl-2-Pyrrolidone
PE petroleum ether
ppm parts per million
RM reaction mixture
TEA Triethylamine
TFA trifluoroacetic acid
THF Tetrahydrofuran
TLC thin layer chromatography
Y Yield

### Example A. 2,8-dimethylimidazo[1,2-a]pyrazin-6-amine

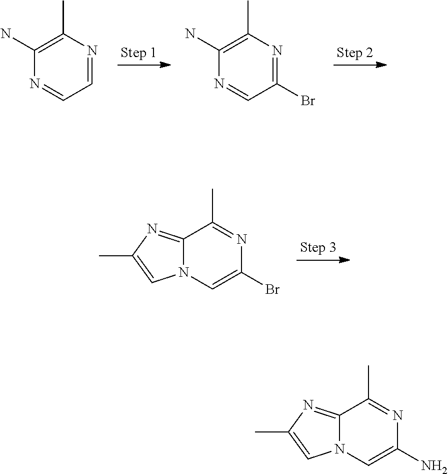

### Step 1: Preparation of 5-bromo-3-methylpyrazin-2-amine

3-methylpyrazin-2-amine (25.37 g, 174.26 mmol) and pyridine (15.16 g, 191.68 mmol, 15.5 mL) were mixed in CHCl₃ (300 mL), and bromine (29.24 g, 182.96 mmol, 9.38 mL) was added dropwise, the mixture was left to stir at r.t. overnight. After that the resulting mixture was washed with water and brine and evaporated under reduced pressure to give pure 5-bromo-3-ethylpyrazin-2-amine (30.08 g, Y: 92%). ESI-MS (M+H)⁺: 189.0. ¹H NMR (500 MHZ, DMSO-d₆) δ 7.87 (s, 1H), 6.4 (br s, 2H), 2.25 (s, 3H).

### Step 2: Preparation of 6-bromo-2,8-dimethylimidazo[1,2-a]pyrazine

To a mixture of 4-methylbenzene-1-sulfonic acid hydrate (1.49 g, 7.82 mmol) and pyridine (618.53 mg, 7.82 mmol, 630.0 μL) in i-PrOH (100 mL) was added 5-bromo-3-methylpyrazin-2-amine (14.71 g, 78.2 mmol) and 1-bromo-2,2-dimethoxypropane (16.46 g, 89.93 mmol, 12.16 mL) and the reaction mixture was heated to 90° C. for 4 h. The resulting mixture was diluted with DCM, washed with a saturated sodium hydrogen carbonate solution, dried (Na₂SO₄) and concentrated under reduced pressure to give pure 6-bromo-2,8-dimethylimidazo[1,2-a]pyrazine (15.3 g, Y: 68%). ¹H NMR (400 MHZ, DMSO-d₆) δ 9.03 (s, 1H), 8.08 (s, 1H, 2.76 (s, 3H), 2.51 (s, 3H).

### Step 3: Preparation of 2,8-dimethylimidazo[1,2-a]pyrazin-6-amine

6-Bromo-2,8-dimethylimidazo[1,2-a]pyrazine (10.0 g, 44.23 mmol), tert-butyl carbamate (4.35 g, 37.16 mmol), Pd₂ (dba); (1.42 g, 1.55 mmol). [5-(diphenylphosphanyl)-9,9-dimethyl-9H-xanthen-4-yl]diphenylphosphane (2.69 g, 4.64 mmol), and cesium carbonate (20.18 g, 61.93 mmol) were suspended in degassed anhydrous 1,4-dioxane. The mixture was heated under N₂ at 100° C. overnight. After cooling to room temperature, the mixture was diluted with ethyl acetate, the organic layer was washed with water and saturated brine, dried over magnesium sulfate, filtered, and concentrated in vacuo. The residue was purified with silica gel column chromatography to afford the tert-butyl N-2,8-dimethylimidazo[1,2-a]pyrazin-6-ylcarbamate, which was treated with HCl/dioxane 10% solution to give 2,8-dimethylimidazo[1,2-a]pyrazin-6-amine as HCl salt (5.56 g, Y: 63%). ESI-MS (M+H)⁺: 163.0. ¹H NMR (400 MHZ, DMSO-d₆) δ 8.02 (s, 1H), 7.71 (s, 1H), 5.20 (br s, 2H), 2.68 (s, 3H), 2.43 (s, 3H).

### Example B. N-(2,8-dimethylimidazo[1,2-a]pyrazin-6-yl)-4-ethoxy-2-(methylsulfinyl)pyrimidine-5-carboxamide

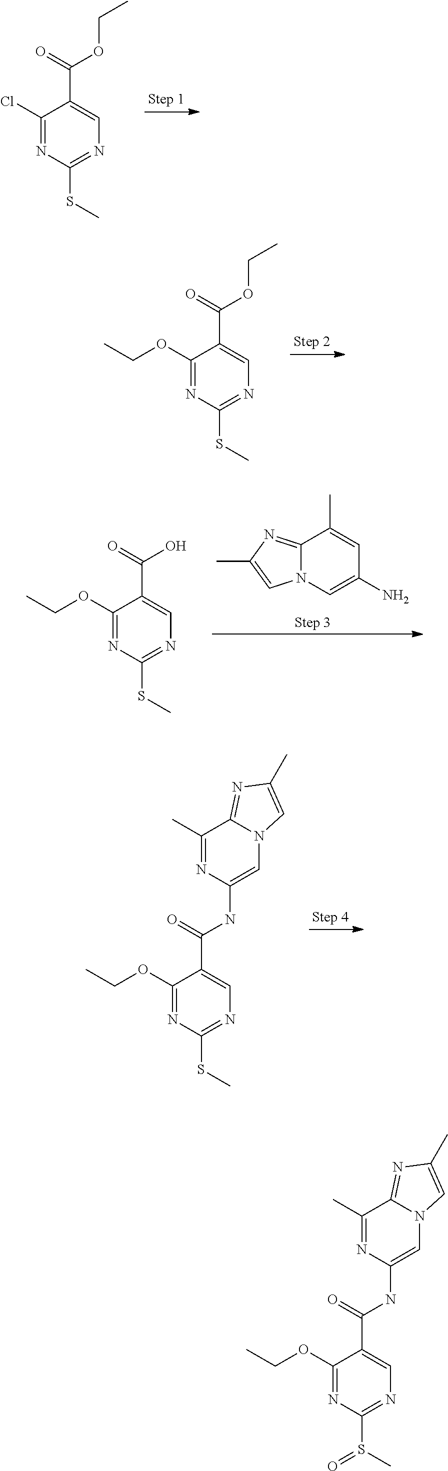 [SMILES: CCOC(=O)c1cnc(SC)nc1Cl ; CCOC(=O)c1cnc(SC)nc1OCC ; CCOc1nc(SC)ncc1C(=O)O ; Cc1cn2cc(N)cc(C)c2n1 ; CCOc1nc(SC)ncc1C(=O)Nc1cn2cc(C)nc2c(C)n1 ; CCOc1nc(S(C)=O)ncc1C(=O)Nc1cn2cc(C)nc2c(C)n1]

### Step 1: Preparation of ethyl 4-ethoxy-2-(methylsulfanyl)pyrimidine-5-carboxylate

An ice-cooled suspension of ethyl 4-chloro-2-(methylsulfanyl)pyrimidine-5-carboxylate 1 (30.0 g, 128.93 mmol) in 300 ml of ethanol was treated dropwise with a solution of sodium ethoxide prepared from sodium (3.26 g, 141.82 mmol) and 300 ml of ethanol. After 1 hour at rt the mixture was evaporated and the residue partitioned between 400 ml of dichloromethane and 400 ml of water. The organic phase was separated, dried over magnesium sulfate, filtered and evaporated to give ethyl 4-ethoxy-2-(methylsulfanyl)pyrimidine-5-carboxylate (31.0 g, Y: 94%) as a colorless oil. ¹H NMR (400 MHz, Chloroform-d₆) δ: 8.81 (s, 1H), 4.56 (q, J=6.8 Hz, 2H), 4.35 (q, J=6.8 Hz, 2H), 2.58 (s, 3H), 1.46 (t, J=7.1 Hz, 3H), 1.38 (t, J=7.1 Hz, 3H)

### Step 2: Preparation of 4-ethoxy-2-(methylsulfanyl)pyrimidine-5-carboxylic acid

A mixture of the ethyl 4-ethoxy-2-(methylsulfanyl)pyrimidine-5-carboxylate 2 (31.0 g, 127.94 mmol) and lithium hydroxide monohydrate (8.05 g, 191.92 mmol) was stirred in a mixture of ethanol (100 mL) and H₂O (40 ml) overnight at rt. The reaction mixture was then concentrated under reduced pressure to remove ethanol, and the resulting aqueous solution was neutralized with 10% NaHSO to pH 5 and extracted with EtOAc (2*100 mL). The organic layers were combined, dried over Na₂SO₄ and evaporated to give pure 4-ethoxy-2-(methylsulfanyl)pyrimidine-5-carboxylic acid (22.0 g, Y: 76.2%). ESI-MS (M+H)⁺: 215.2. ¹H NMR (400 MHZ, DMSO-d₆) &: 8.43 (s, 1H), 4.39 (q, J=6.8 Hz, 2H), 2.47 (s, 3H), 1.29 (t, J=7.1 Hz, 3H).

### Step 3: Preparation of N-(2,8-dimethylimidazo[1,2-a]pyrazin-6-yl)-4-ethoxy-2-(methylthio)pyrimidine-5-carboxamide

The 4-ethoxy-2-(methylsulfanyl)pyrimidine-5-carboxylic acid (9.86 g, 46.01 mmol) was suspended in DMF (150 mL) and ethylbis(propan-2-yl)amine (11.89 g, 92.03 mmol) was added followed by [(dimethylamino) (3H-[1,2,3]triazolo[4,5-b]pyridin-3-yloxy)methylidene]dimethylazanium; hexafluoro-lambda5-phosphanuide (HATU) (20.99 g. 55.22 mmol). The resulting mixture was stirred for 30 min at rt. After that 2,8-dimethylimidazo[1,2-a]pyrazin-6-amine (9.13 g, 46.01 mmol) was added in one portion and the reaction mixture was stirring overnight at rt. The precipitate formed was filtered, washed with MeCN (60 ml), MTBE (60 mL), dried under vacuo to give N-(2,8-dimethylimidazo[1,2-a]pyrazin-6-yl)-4-ethoxy-2-(methylthio)pyrimidine-S-carboxamide (11.82 g, Y: 72%), ¹H NMR (400 MHZ, Chloroform-d)δ:9.8 (s, 1H), 9.13 (s, 1H), 9.10 (s, 1H), 7.44 (s, 1H), 4.75 (q. J=6.8 Hz), 2.82 (s, 3H), 2.61 (s, 3H), 2.50 (s, 3H), 1.63 (t, J=7.1 Hz).

### Step 4: Preparation of N-(2,8-dimethylimidazo[1,2-a]pyrazin-6-yl)-4-ethoxy-2-(methylsulfinyl)pyrimidine-5-carboxamide

To a solution of N-(2,8-dimethylimidazo[1,2-a]pyrazin-6-yl)-4-ethoxy-2-(methylthio)pyrimidine-5-carboxamide (11.82 g, 33.0 mmol) in dichloromethane (200 mL) was added 3-chlorobenzene-1-carboperoxoic acid (7.12 g, 41.2 mmol) (75% purity) dropwise over 1 h at 0° C., and the reaction mixture was stirred at room temperature for 1 h. The crude reaction mixture was poured onto NaOH (1 M, 100 ml), and the organic material extracted twice with dichloromethane. The combined organic layers were washed with brine, dried over MgSO₄, filtered, and the filtrate concentrated under reduced pressure to afford crude product which was purified by crystallization from minimum amount of MTBE to give the N-(2,8-dimethylimidazo[1,2-a]pyrazin-6-yl)-4-ethoxy-2-(methylsulfinyl)pyrimidine-5-carboxamide (9.27 g, Y: 75%). ESI-MS (M+H)⁺: 375.0. ¹H NMR (400 MHZ, Chloroform-d) 8:9.85 (s, 1H), 9.44 (s, 1H), 9.13 (s, 1H), 7.49 (s, 1H), 4.90 (q, J=6.8 Hz, 2H), 3.0 (s, 3H), 2.54 (s, 3H), 2.53 (s, 3H), 1.68 (t, J=J=7.1 Hz, 3H).

### Example C. 2-4-[(tert-butoxy) carbonyl]piperazin-1-yl-4-ethoxypyrimidine-5-carboxylic acid

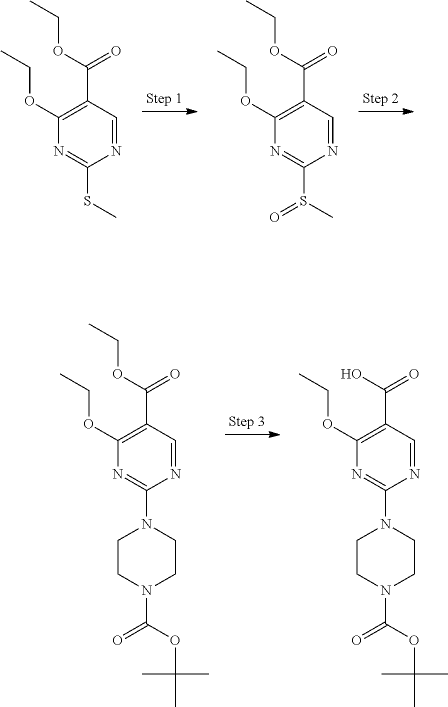 [SMILES: CCOC(=O)c1cnc(SC)nc1OCC ; CCOC(=O)c1cnc(S(C)=O)nc1OCC ; CCOC(=O)c1cnc(N2CCN(C(=O)OC(C)(C)C)CC2)nc1OCC ; CCOc1nc(N2CCN(C(=O)OC(C)(C)C)CC2)ncc1C(=O)O]

### Step 1: Preparation of ethyl 4-ethoxy-2-methane sulfinylpyrimidine-5-carboxylate

To a solution of the ethyl 4-ethoxy-2-(methylsulfanyl)pyrimidine-5-carboxylate (15.0 g, 61.91 mmol) in chloroform (150 mL), 3-chlorobenzene-1-carboperoxoic acid (MCPBA) (12.82 g, 74.29 mmol) was added in portions under ice-cooling, and the reaction mixture was stirred for 2 hours. The resulting mixture was filtered, and the filtrate was washed with a saturated aqueous sodium hydrogen carbonate solution (100 mL) and brine (50 mL). The organic layer was dried over anhydrous sodium sulfate, and the solvent was evaporated in vacuo to give crude ethyl 4-ethoxy-2-methanesulfinylpyrimidine-S-carboxylate (15.75 g, Y.: 78.8%). ¹H NMR (500 MHz, DMSO-d₆) δ 9.01 (s, 1H), 4.54 (q, J=7.2 Hz 2H), 4.30 (q, J=7.2 Hz, 2H), 2.88 (s, 3H), 1.35 (t, J=7.1 Hz, 3H), 1.29 (t, J=7.1 Hz, 3H).

### Step 2: Preparation of ethyl 2-4-[(tert-butoxy)carbonyl]piperazin-1-yl-4-ethoxypyrimidine-5-carboxylate

tert-Butyl piperazine-1-carboxylate (11.36 g, 60.98 mmol) was added dropwise to a cooled to 0° C. suspension of ethyl 4-ethoxy-2-methanesulfinylpyrimidine-5-carboxylate (15.75 g, 60.98 mmol) and potassium carbonate (25.28 g, 182.93 mmol) in MeCN (200 mL), and the reaction mass was stirred overnight at r.t. Then the resulting mixture was filtered and MeCN was evaporated under reduced pressure. The residue was partitioned between EtO Ac and H₂O. The organic layer was dried over anhydrous MgSO₄, filtered and concentrated in vacuo. The residue was purified by flash chromatography (4/1; Hex/EtOAc) to give ethyl 2-4-[(tert-butoxy) carbonyl]piperazin-1-yl-4-ethoxypyrimidine-5-carboxylate (7.0 g, Y.: 28.7%). ¹H NMR (400 MHZ, DMSO-d₆) δ 8.61 (s, 1H), 4.39 (q, J=7.2 Hz, 2H), 4.17 (q, J=7.2 Hz, 2H), 3.85-3.75 (m, 4H), 3.45-3.35 (m, 4H), 1.41 (s, 9H), 1.31 (t, J=7.1 Hz, 3H), 1.24 (t, J=7.1 Hz, 3H).

### Step 3: Preparation of 2-4-[(tert-butoxy) carbonyl]piperazin-1-yl-4-ethoxypyrimidine-5-carboxylic acid

A mixture of ethyl 2-4-[(tert-butoxy) carbonyl]piperazin-1-yl-4-ethoxypyrimidine-5-carboxylate (7.0 g, 18.4 mmol) and lithium hydroxide monohydrate (926.57 mg, 22.08 mmol) was stirred overnight in a mixture of ethanol (100 mL) and H₂O) (40 mL). The reaction mixture was then concentrated under reduced pressure to remove ethanol, and the resulting aqueous solution was neutralized with 10% NaHSO₄ to pH=5 and extracted with EtOAc (2×100 mL). The organic layers were combined, dried over Na₂SO₄ and evaporated under reduced pressure to give pure 2-4-[(tert-butoxy) carbonyl]piperazin-1-yl-4-ethoxypyrimidine-5-carboxylic acid (6.15 g, Y.: 90.1%). ESI-MS (M+H)⁺: 353.0. ¹H NMR (400 MHZ, DMSO-d₆)δ 12.28 (br s, 1H), 8.61 (s, 1H), 4.40 (q, J=7.2 Hz, 2H), 3.83-3.75 (m, 4H), 3.45-3.35 (m, 4H), 1.42 (s, 9H), 1.32 (t, J=7.1 Hz, 3H).

### Example D. tert-butyl 4-(5-carbamoyl-4-ethoxypyrimidin-2-yl) piperazine-1-carboxylate

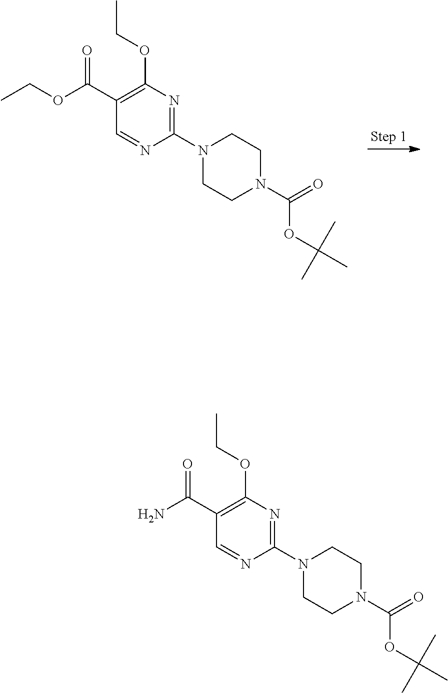 [SMILES: CCOC(=O)c1cnc(N2CCN(C(=O)OC(C)(C)C)CC2)nc1OCC ; CCOc1nc(N2CCN(C(=O)OC(C)(C)C)CC2)ncc1C(N)=O]

### Step 1: Preparation of tert-butyl 4-(5-carbamoyl-4-ethoxypyrimidin-2-yl) piperazine-1-carboxylate

Ethyl 2-4-[(tert-butoxy) carbonyl]piperazin-1-yl-4-ethoxypyrimidine-5-carboxylate (1.0 g, 2.62 mmol) was dissolved in NH₃/MeOH (20 mL) and the reaction mixture was sealed and heated to 70° C. overnight. The reaction mixture was cooled and evaporated in vacuo to give the title product (883 mg, Y: 95%). ESI-MS (M+H)⁺: 352.4.

### Example E. 4-ethoxy-N-8-fluoro-2-methylimidazo[1,2-a]pyridin-6-yl-2-methanesulfinylpyrimidine-5-carboxamide

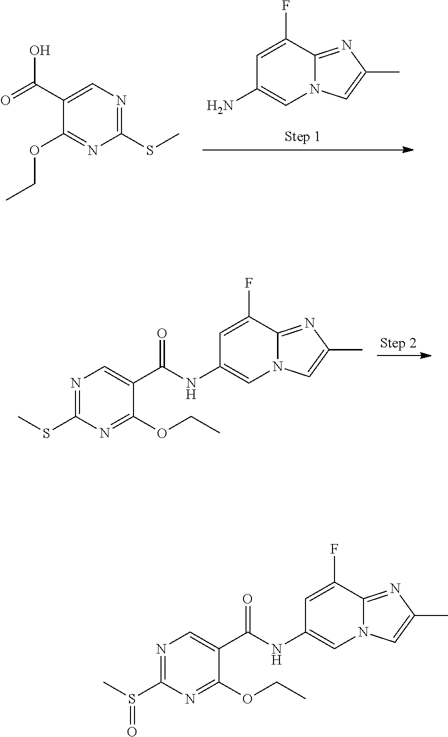

### Step 1: Preparation of 4-ethoxy-N-8-fluoro-2-methylimidazo[1,2-a]pyridin-6-yl-2-(methylsulfanyl)-pyrimidine-5-carboxamide

4-Ethoxy-2-(methylsulfanyl)pyrimidine-5-carboxylic acid (9.86 g, 46.01 mmol) was suspended in MeCN (200 mL) and ethylbis(propan-2-yl)amine (11.89 g, 92.03 mmol) was added followed by [(dimethylamino) (3H-[1,2,3]triazolo[4,5-b]pyridin-3-yloxy)methylidene]dimethylazanium; hexafluoro-lambda5-phosphanuide (HATU) (20.99 g, 55.22 mmol). The mixture was stirred for 30 min at r.t. After that 8-fluoro-2-methylimidazo[1,2-a]pyridin-6-amine (7.6 g, 46.01 mmol) was added in one portion and the reaction mixture was stirring overnight at r.t. The precipitate formed was filtered, washed with MeCN (60 mL), MTBE (60 mL), and dried in vacuo to give pure 4-ethoxy-N-8-fluoro-2-methylimidazo[1,2-a]pyridin-6-yl-2-(methylsulfanyl)pyrimidine-5-carboxamide (6.6 g, Y.: 37.7%). ESI-MS (M+H)⁺: 362.0. ¹H NMR (400 MHz, DMSO-d₆) δ 10.10 (s, 1H), 9.07 (s, 1H), 8.68 (s, 1H), 7.90 (d, J=3.1 Hz, 1H), 7.15 (d, J=12.3 Hz, 1H), 4.53 (q, J=7.2, 2H), 2.56 (s, 3H), 2.32 (s, 3H), 1.39 (t, J=7.1, 3H).

### Step 2: Preparation of 4-ethoxy-N-8-fluoro-2-methylimidazo[1,2-a]pyridin-6-yl-2-methanesulfinyl-pyrimidine-5-carboxamide

To a solution of 4-ethoxy-N-8-fluoro-2-methylimidazo[1,2-a]pyridin-6-yl-2-(methylsulfanyl)pyrimidine-5-carboxamide (6.63 g, 18.35 mmol) in dichloromethane was added dropwise 3-chlorobenzene-1-carboperoxoic acid (3.8 g, 22.02 mmol) (75% purity) over 1 h at 0° C., and the reaction mixture was stirred at room temperature for 1 h. The crude reaction mixture was poured onto NaOH (1 M, 100 mL), and the organic material was extracted twice with dichloromethane. The combined organic layers were washed with brine, dried over MgSO₄, filtered, and the filtrate was concentrated under reduced pressure to afford the crude product which was purified by crystallization from minimum amount of MTBE to give 4-ethoxy-N-8-fluoro-2-methylimidazo[1,2-a]pyridin-6-yl-2-methanesulfinylpyrimidine-5-carboxamide (5.2 g, Y.: 71.3%). ESI-MS (M+H)⁺: 378.0. ¹H NMR (400 MHz, DMSO-d₆) δ 10.50 (s, 1H), 9.10 (s, 1H), 8.95 (s, 1H), 7.94 (d, J=3.2 Hz, 1H), 7.12 (d, J=12.3 Hz, 1H), 4.58 (q, J=7.2 Hz, 2H), 2.91 (s, 3H), 2.33 (s, 3H), 1.38 (t, J=7.1, 3H).

### Example 1. N-(8-fluoro-2-methylimidazo[1,2-a]pyridin-6-yl)-4-methoxy-2-(piperazin-1-yl)pyrimidine-5-carboxamide, HCl (Compound 1)

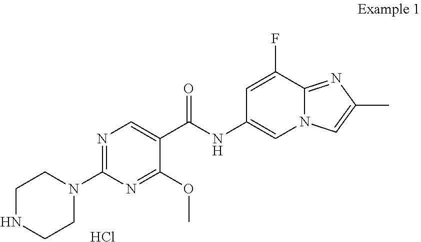 [SMILES: COc1nc(N2CCNCC2)ncc1C(=O)Nc1cc(F)c2nc(C)cn2c1 ; Cl]

### Step 1: Synthesis of methyl 4-methoxy-2-(methylthio)pyrimidine-5-carboxylate

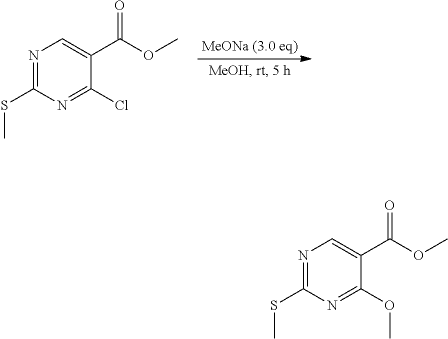

To a stirred solution of methyl 4-chloro-2-(methylthio)pyrimidine-5-carboxylate (5.0 g, 21.5 mmol) in MeOH (50 mL) under argon atmosphere was added NaOMe (3.61 g, 64.5 mmol) and the mixture was stirred at rt for 5 h. The reaction mixture was quenched with acetic acid (10 mL). After concentration under reduced pressure, the residue was purified by silica gel column chromatography (PE/EA=4:1) to give methyl 4-methoxy-2-(methylthio)pyrimidine-5-carboxylate (3.85 g, Y: 83.7%) as a white solid. ¹H NMR (400 MHZ, DMSO-d₆) δ 8.75 (s, 1H), 4.01 (s, 3H), 3.81 (s, 3H), 2.57 (s, 3H). ESI-MS (M+H)⁺: 215.1.

### Step 2: Synthesis of methyl 4-methoxy-2-(methylsulfinyl)pyrimidine-5-carboxylate

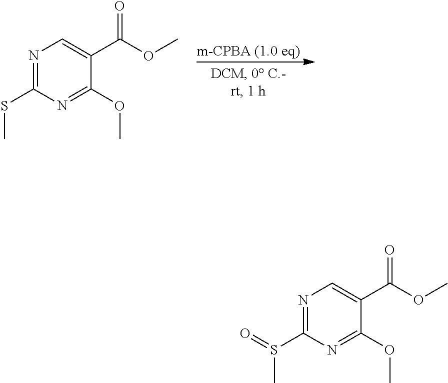

To a stirred solution of methyl 4-methoxy-2-(methylthio)pyrimidine-5-carboxylate (200 mg, 0.93 mmol) in DCM (10 mL) was added m-CPBA (161 mg, 0.93 mmol) at 0° C. The mixture was warmed to rt and stirred for 1 h. The reaction mixture was diluted with DCM (50 mL) and quenched with sat. aqueous Na₂SO₃ (10 mL). The organic phase was washed with sat. aqueous sodium carbonate (10 mL), dried over sodium sulfate. After concentration under reduced pressure, crude methyl 4-methoxy-2-(methylsulfinyl)pyrimidine-5-carboxylate (200 mg, Y: 93%) as a white solid, which was used to next step without further purification. ESI-MS (M+H)⁺: 230.9.

### Step 3: Synthesis of methyl 2-(4-(tert-butoxycarbonyl) piperazin-1-yl)-4-methoxypyrimidine-5-carboxylate

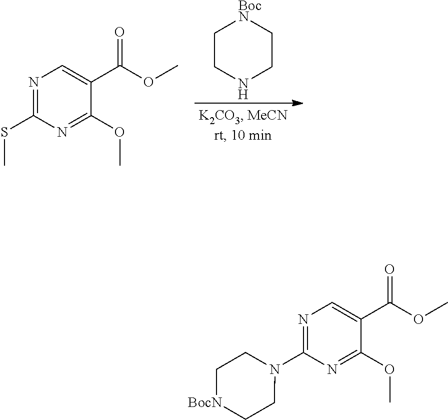

To a stirred solution of methyl 4-methoxy-2-(methylsulfinyl)pyrimidine-5-carboxylate (200 mg, 0.87 mmol) in MeCN (8 mL) was added tert-butyl piperazine-1-carboxylate (323 mg, 1.74 mmol) and potassium carbonate (236 mg, 1.74 mmol). The mixture was stirred for 10 min. After concentration, the residue was treated with EA/Water (20 mL, 1:1). The organic phase was separated and the aqueous phase was extracted with EA (10 mL×3). The combined organics dwas dried over sodium sulfate. After concentration under reduced pressure, crude methyl 2-(4-(tert-butoxycarbonyl) piperazin-1-yl)-4-methoxypyrimidine-5-carboxylate (300 mg, Y: 96%) as a white solid, which was used to next step without further purification. ESI-MS (M+H)⁺: 353.0.

### Step 4: Synthesis of 2-(4-(tert-butoxycarbonyl) piperazin-1-yl)-4-methoxypyrimidine-5-carboxylic acid

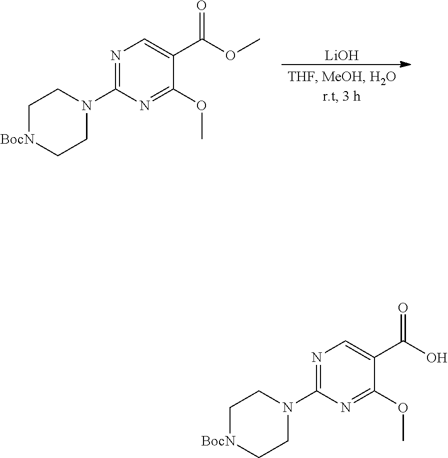 [SMILES: COC(=O)c1cnc(N2CCN(C(=O)OC(C)(C)C)CC2)nc1OC ; "LiOH" ; COc1nc(N2CCN(C(=O)OC(C)(C)C)CC2)ncc1C(=O)O]

To a solution of methyl 2-(4-(tert-butoxycarbonyl) piperazin-1-yl)-4-methoxypyrimidine-5-carboxylate (150 mg, 0.426 mmol) in THF (8 mL), MeOH (8 ml) and H₂O (4 ml) was added LiOH.H₂O (100 mg, 2.13 mmol) at rt. The reaction mixture was stirred for 3 h at rt. After concentration, the residue was diluted with water (20 mL) and adjusted pH to 5-6 with 1 N HCl aqueous solution. The solid was formed and collected by filtration, washed with water for three times (3 mL×3). The solid was dried under vacuum at 55° C. to provide title product as yellow solid (140 mg, Y: 97%). ¹H NMR (400 MHZ, CDCl₃)δ 8.86 (s, 1H), 4.09 (s, 3H), 3.91 (s, 4H), 3.54-3.50 (m, 4H), 1.49 (s, 9H). ESI-MS (M+H)⁺: 339.0.

### Step 5: Synthesis of 6-bromo-8-fluoro-2-methylimidazo[1,2-a]pyridine

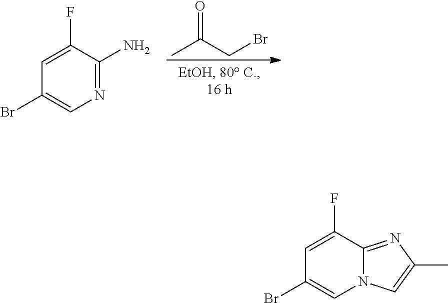

A mixture of 4-1 (10 g, 52.6 mmol) and 1-bromopropan-2-one (17.9 g, 131.6 mmol) in EtOH (80 mL) was heated to 80° C. for 16 h. LCMS showed 15% of SM was left and 50% of DP was formed. The reaction was stopped and cooled to r.t. The solvent was removed under vacuum and the residue was purified by silica gel column chromatography (PE/EA=4:1) to give 6-bromo-8-fluoro-2-methylimidazo[1,2-a]pyridine (5.0 g, Y: 41.7%) as light pink solid.

¹H NMR (400 MHZ, CDCl₃)δ 8.03 (d, J=1.4 Hz, 1H), 7.38 (d, J=1.4 Hz, 1H), 6.97 (dd, J=9.6, 1.5 Hz, 1H), 2.47 (s, 3H). ESI-MS (M+H)⁺: 229.1, 231.1.

### Step 6: Synthesis of N-(8-fluoro-2-methylimidazo[ 1,2-a]pyridin-6-yl)-1,1-diphenylmethanimine

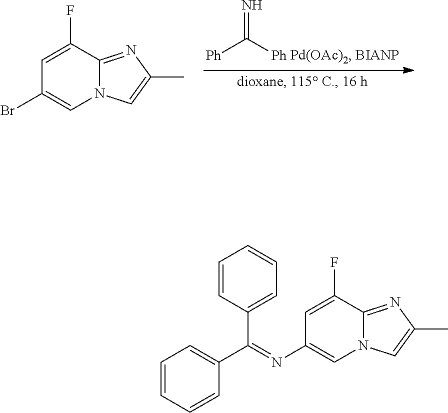

A mixture of 6-bromo-8-fluoro-2-methylimidazo[1,2-a]pyridine (1.5 g, 6.58 mmol), diphenylmethanimine (1.25 g, 6.91 mmol), Pd(OAc)₂ (148 mg, 0.658 mmol), BINAP (818 mg, 1.316 mmol) and Cs₂CO₃ (4.28 g, 13.16 mmol) in dioxane (70 mL) was purged with N₂ for three times at rt. Then the mixture was heated to 115° C. for 16 h. The solvent was removed and the residue was purified by silica gel column (0˜50% EA in PE) to provide title product as light brown solid (336 mg (pure)+400 mg (impure), yield: 35%). ESI-MS (M+H)⁺: 330.1.

### Step 7: Synthesis of 8-fluoro-2-methylimidazo[1,2-a]pyridin-6-amine hydrochloride

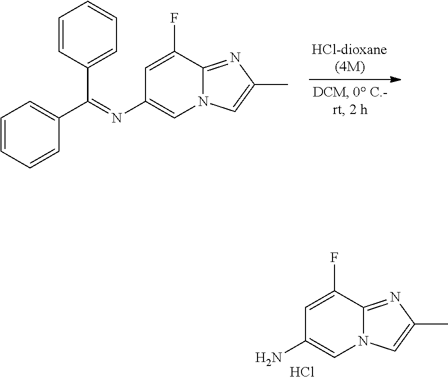 [SMILES: Cc1cn2cc(N=C(c3ccccc3)c3ccccc3)cc(F)c2n1 ; Cc1cn2cc(N)cc(F)c2n1 ; Cl]

To a solution of N-(8-fluoro-2-methylimidazo[1,2-a]pyridin-6-yl)-1,1-diphenylmethanimine (336 mg, 1.02 mmol) in DCM (10 mL) at 0° C. was added HCl-dioxane (4M, 2.6 mL, 10.2 mmol). The mixture was stirred for 2 h at rt. After concentration, the residue was treated with EA. The solid was collected by filtration and washed with EA and dried under vacuum to provide title product as yellow solid (180 mg, yield: 90%). ¹H NMR (400 MHZ, DMSO-d₆) δ 8.05 (dd, J=2.2, 1.0 Hz, 1H), 7.87 (d, J=1.6 Hz, 1H), 7.40 (dd, J=12.3, 1.6 Hz, 1H), 5.35 (s, 3H), 2.43 (d, J=0.9 Hz, 3H).

### Step 8: Synthesis of tert-butyl 4-(5-((8-fluoro-2-methylimidazo[1,2-a]pyridin-6-yl) carbamoyl)-4-methoxypyrimidin-2-yl) piperazine-1-carboxylate

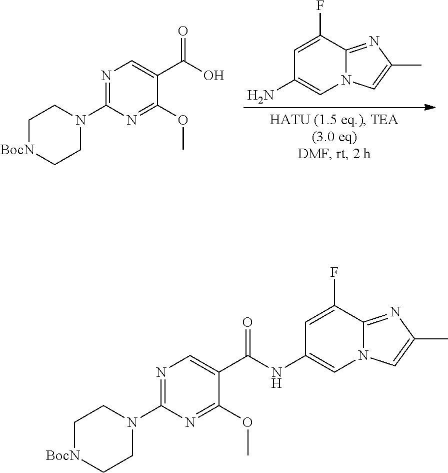 [SMILES: COc1nc(N2CCN(C(=O)OC(C)(C)C)CC2)ncc1C(=O)O ; Cc1cn2cc(N)cc(F)c2n1 ; COc1nc(N2CCN(C(=O)OC(C)(C)C)CC2)ncc1C(=O)Nc1cc(F)c2nc(C)cn2c1]

To a stirred solution of 2-(4-(tert-butoxycarbonyl) piperazin-1-yl)-4-methoxypyrimidine-5-carboxylic acid (120 mg, 0.36 mmol) in DMF (5 mL) was added 8-fluoro-2-methylimidazo[1,2-a]pyridin-6-amine (107 mg, 0.53 mmol), TEA (107 mg, 1.07 mmol) and HATU (202 mg, 0.53 mmol). The mixture was stirred for 2 h at rt. Water was added and the precipitate was collected by filteration. The filter cake was washed with water and dried under vacuum to provide tert-butyl 4-(5-((8-fluoro-2-methylimidazo[1,2-a]pyridin-6-yl) carbamoyl)-4-methoxypyrimidin-2-yl) piperazine-1-carboxylate (100 mg, Y: 58%) as a gray solid, which was used to next step without further purification. ESI-MS (M+H)⁺: 486.1.

### Step 9: Synthesis of N-(8-fluoro-2-methylimidazo[1,2-a]pyridin-6-yl)-4-methoxy-2-(piperazin-1-yl)pyrimidine-5-carboxamide, HCl

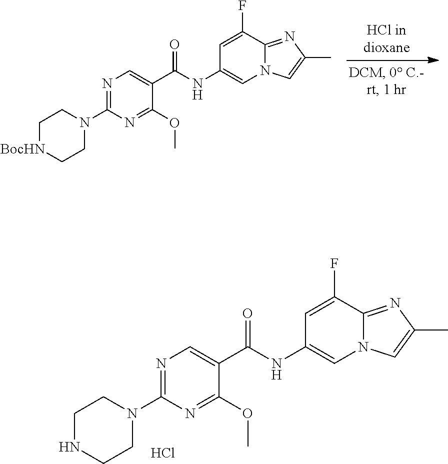

To a solution of tert-butyl 4-(5-((8-fluoro-2-methylimidazo[1,2-a]pyridin-6-yl) carbamoyl)-4-methoxypyrimidin-2-yl) piperazine-1-carboxylate (90 mg, 0.19 mmol) in DCM (10 mL) was added HCl solution in 1,4-dioxane (1 mL, 4 mmoL, 4M) at 0° C. The the mixture was stirred at rt for 1 h.After concentration, the residue was purified by pre-HPLC (MeCN/0.05% HCl in water) to give N-(8-fluoro-2-methylimidazo[1,2-a]pyridin-6-yl)-4-methoxy-2-(piperazin-1-yl)pyrimidine-5-carboxamide as the HCl salt (48 mg, Y: 62%) as a gray solid. ¹H NMR (400 MHZ, CD3OD) δ 9.44 (d, J=1.1 Hz, 1H), 8.86 (s, 1H), 8.10 (dd, J=2.2, 1.1 Hz, 1H), 8.01 (dd, J=11.5, 1.5 Hz, 1H), 4.24-4.17 (m, 7H), 3.36-3.32 (m, 4H), 2.57 (d, J=1.0 Hz, 3H). ESI-MS (M+H)⁺: 386.1.

### Example 2. 4-ethoxy-N-{8-fluoro-2-methylimidazo[1,2-a]pyridin-6-yl}-2-(piperazin-1-yl)pyrimidine-5-carboxamide (Compound 2)

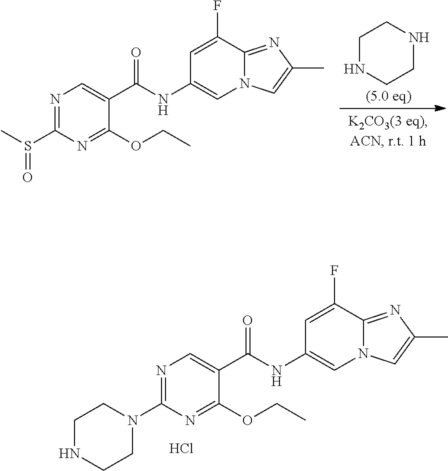

### Step 1: Preparation of 4-ethoxy-N-(8-fluoro-2-methylimidazo[1,2-a]pyridin-6-yl)-2-(piperazin-1-yl)pyrimidine-5-carboxamide hydrochloride

To a solution of 4-ethoxy-N-(8-fluoro-2-methylimidazo[1,2-a]pyridin-6-yl)-2-(methylsulfinyl)pyrimidine-5-carboxamide (50 mg, 0.13 mmol) in ACN (5 mL) was added piperazine (56 mg, 0.65 mmol) and K₂CO₃ (53 mg, 0.39 mmol). The reaction mixture was stirred for 1 h at rt. The solid was filtered and the filtratrate was concentrated. The residue was purified by pre-HPLC (MeCN/0.05% HCl in water) to give title product (22 mg, Y: 39%) as a yellow solid. ESI-MS (M+H)⁺: 400.1.

¹H NMR (400 MHZ, MeOD)δ 9.01 (d, J=1.4 Hz, 1H), 8.82 (s, 1H), 7.73 (d, J=2.3 Hz, 1H), 7.17 (dd, J=11.8, 1.5 Hz, 1H), 4.67-4.61 (m, 2H), 4.22-4.10 (m, 4H), 3.37-3.31 (m, 4H), 2.42 (s, 3H), 1.54 (t, J=7.1 Hz, 3H).

### Example 3. 4-ethoxy-N-{8-fluoro-2-methylimidazo[1,2-a]pyridin-6-yl}-2-[(3R)-3-methylpiperazin-1-yl]pyrimidine-5-carboxamide (Compound 3)

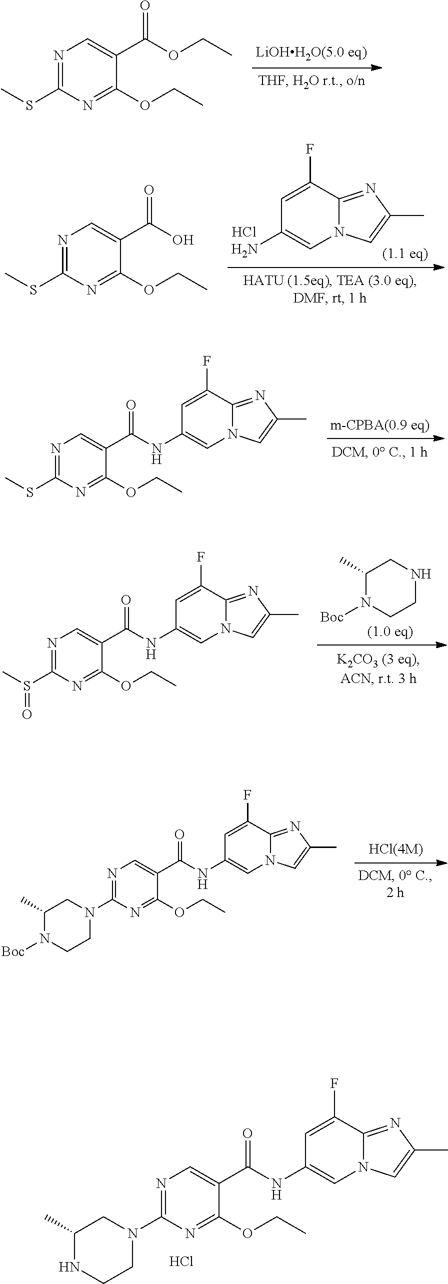

### Step 1: synthesis of 4-ethoxy-2-(methylthio)pyrimidine-5-carboxylic acid

To a solution of ethyl 4-ethoxy-2-(methylthio)pyrimidine-5-carboxylate (8.4 g, 34.71 mmol) in THF (100 mL) and H₂O (20 mL) was added LiOH H₂O (6.9 g, 173.55 mmol) at rt. The reaction mixture was stirred overnight at rt. The solvent was removed under vacuum. The residue was diluted with water (20 mL) and adjusted to pH=5˜6 with 2 N HCl. The solid was formed and collected by filtration, washed with water three times (5 ml×3). The solid was dried under vacuum at 55° C. to provide 4-ethoxy-2-(methylthio)pyrimidine-5-carboxylic acid (7.1g, Y: 95%) as a white solid. ESI-MS (M+H)⁺: 214.9. ¹H NMR (400 MHz, DMSO-d₆) δ 13.00 (s, 1H), 8.74 (s, 1H), 4.49 (m, 2H), 2.54 (s, 3H), 1.34 (t, J=7.2, 3H).

### Step 2: synthesis of 4-ethoxy-N-(8-fluoro-2-methylimidazo[1,2-a]pyridin-6-yl)-2-(methylthio)pyrimidine-5-carboxamide

To a mixture of 4-ethoxy-2-(methylthio)pyrimidine-5-carboxylic acid (500 mg, 2.33 mmol) in DMF (10 mL) was added 8-fluoro-2-methylimidazo[1,2-a]pyridin-6-amine (422 mg, 2.56 mmol), DIEA (902 mg, 6.99 mmol) and HATU (1.3 g, 3.49 mmol) at 0° C. The mixture was stirred at rt for 1 h and quenched with water (10 mL). The mixture was filtered and the cake was washed with EtOAc (5 mL), dried under vacuum. The product 4-ethoxy-N-(8-fluoro-2-methylimidazo[1,2-a]pyridin-6-yl)-2-(methylthio)pyrimidine-5-carboxamide (360 mg. Y: 59%) was obtained as a yellow solid. ESI-MS (M+H)⁺: 362.1.1H NMR (400 MHZ, DMSO-d₆) δ 10.16 (s, 1H), 9.11 (s, 1H), 8.69 (s, 1H), 7.95 (s, 1H), 7.21 (d, J=12.2 Hz, 1H), 4.55 (q, J=7.0 Hz, 2H), 2.57 (s, 3H), 2.35 (s, 3H), 1.40 (t, J=7.0 Hz, 3H).

### Step 3: synthesis of 4-ethoxy-N-(8-fluoro-2-methylimidazo[1,2-a]pyridin-6-yl)-2-(methylsulfinyl)pyrimidine-5-carboxamide

To a mixture of 4-ethoxy-N-(8-fluoro-2-methylimidazo[1,2-a]pyridin-6-yl)-2-(methylthio)pyrimidine-5-carboxamide (360 mg, 1.00 mmol) in DCM (10 mL) was added m-CPBA (156 mg, 0.90 mmol) at 0° C. The reaction mixture was stirred at 0° C. for 1 h and quenched with saturated aqueous Na₂S₂O₃ solution. The mixture was extracted with DCM (10 mL×3). The combined organics was dried over sodium sulfate and concentrated. The crude 4-ethoxy-N-(8-fluoro-2-methylimidazo[1,2-a]pyridin-6-yl)-2-(methylsulfinyl)pyrimidine-5-carboxamide (360 mg, Y: 95%) was obtained as a yellow solid, which was used in the next step without further purification. ESI-MS (M+H)⁺: 378.0.

### Step 4: synthesis of tert-butyl(R)-4-(4-ethoxy-5-((8-fluoro-2-methylimidazo[1,2-a]pyridin-6-yl) carbamoyl)pyrimidin-2-yl)-2-methylpiperazine-1-carboxylate

To a solution of 4-ethoxy-N-(8-fluoro-2-methylimidazo[1,2-a]pyridin-6-yl)-2-(methylsulfinyl)pyrimidine-5-carboxamide (90 mg, 0.24 mmol) in ACN (5 mL) was added tert-butyl(R)-2-methylpiperazine-1-carboxylate (48 mg, 0.24 mmol) and K₂CO₃ (99 mg, 0.72 mmol). The reaction mixture was stirred for 3 h at rt. After concentration, the residue was purified by silica gel column chromatography (DCM:MeOH=20:1) to give tert-butyl(R)-4-(4-ethoxy-5-((8-fluoro-2-methylimidazo[1,2-a]pyridin-6-yl) carbamoyl)pyrimidin-2-yl)-2-methylpiperazine-1-carboxylate (50 mg, Y: 41%) as a yellow solid. ESI-MS (M+H)⁺: 514.2. ¹H NMR (400 MHZ, CDCl₃)δ 9.21 (s, 1H), 9.10 (d, J=1.6 Hz, 1H), 8.99 (s, 1H), 7.40 (d, J=2.3 Hz, 1H), 6.57 (dd, J=10.9, 1.6 Hz, 1H), 4.62-4.60 (m, 4H), 4.38 (br s, 1H), 3.96 (d, J=9.4 Hz, 1H), 3.29 (d, J=12.1 Hz, 1H), 3.15 (m, 2H), 2.47 (s, 3H), 1.58 (t. J=7.2 Hz, 3H), 1.49 (s, 9H), 1.14 (d, J=6.7 Hz, 3H).

### Step 5: synthesis of (R)-+-ethoxy-N-(8-fluoro-2-methylimidazo[1,2-a]pyridin-6-yl)-2-(3-methylpiperazin-1-yl)pyrimidine-5-carboxamide hydrochloride

To a solution of tert-butyl(R)-4-(4-ethoxy-5-((8-fluoro-2-methylimidazo[1,2-a]pyridin-6-yl) carbamoyl)pyrimidin-2-yl)-2-methylpiperazine-1-carboxylate (45 mg, 0.088 mmol) in DCM (5 mL) was added HCl solution in 1,4-dioxane (1 mL, 4 mmol, 4M) at 0° C. The reaction mixture was stirred at rt for 2 h. After concentration, the residue was purified by pre-HPLC (MeCN/0.05% HCl in water) to give title product (15.25 mg, Y: 39%) as a yellow solid. ESI-MS (M+H)⁺: 414.4. ¹H NMR (400 MHZ, DMSO-d₆)δ 10.37 (s, 1H), 9.80 (d, J=13.5 Hz, 2H), 9.50 (t. J=4.0 Hz, 1H), 8.65 (d, J=10.8 Hz, 1H), 8.31 (s, 1H), 8.08 (d, J=11.9 Hz, 1H), 4.65 (d, J=13.6 Hz, 2H), 4.53 (q. J=7.2, 2H), 3.51 (t, J=11.4 Hz, 1H), 3.32 (m, 3H), 3.02 (d, J=9.6 Hz, 1H), 2.50 (s, 3H), 1.41 (t, J-7.2 Hz, 3H), 1.34 (d, J=5.7 Hz, 3H).

### Example 4. 2-[(3R,5S)-3,5-dimethylpiperazin-1-yl]-4-ethoxy-N-{8-fluoro-2-methylimidazo[1,2-a]pyridin-6-yl}pyrimidine-5-carboxamide hydrochloride (Compound 4)

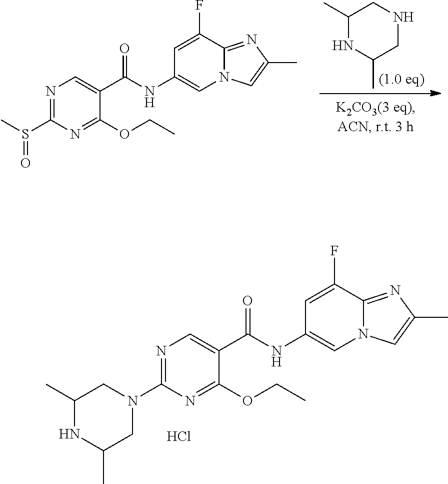

### Step 1: Preparation of 2-(3,5-dimethylpiperazin-1-yl)-4-ethoxy-N-(8-fluoro-2-methylimidazo[1,2-a]pyridin-6-yl)pyrimidine-5-carboxamide hydrochloride

To a solution of 4-ethoxy-N-(8-fluoro-2-methylimidazo[1,2-a]pyridin-6-yl)-2-(methylsulfinyl)pyrimidine-S-carboxamide (50 mg, 0.13 mmol) in ACN (5 mL) was added 2,6-dimethylpiperazine (16 mg, 0.13 mmol) and K₂CO₃ (53 mg, 0.39 mmol). The reaction mixture was stirred for 3 h at rt. The solid was filtered and the filtratrate was concentrated. The residue was purified by pre-HPLC (MeCN/0.05% HCl in water) to give title product (46 mg, Y: 76%) as a yellow solid. ESI-MS (M+H)⁺: 428.4.

¹H NMR (400 MHZ, DMSO)δ 10.37 (s, 1H), 9.80 (br s, 2H), 9.50 (t, J=4.0 Hz, 1H), 8.65 (d, J=10.8 Hz, 1H), 8.31 (s, 1H), 8.08 (d, J=11.9 Hz, 1H), 4.65 (d, J=13.6 Hz, 2H), 4.57-4.52 (m, 2H), 3.51 (t, J=11.4 Hz, 1H), 3.34-3.30 (m, 3H), 3.02 (d, J=9.6 Hz, 1H), 2.50 (s, 3H), 1.41 (t, J=8.2 Hz, 3H), 1.34 (d, J=5.7 Hz, 6H).

### Example 5. 4-ethoxy-N-{8-fluoro-2-methylimidazo[1,2-a]pyridin-6-yl}-2-{octahydropyrrolo[1,2-a]pyrazin-2-yl}pyrimidine-5-carboxamide (Compound 5)

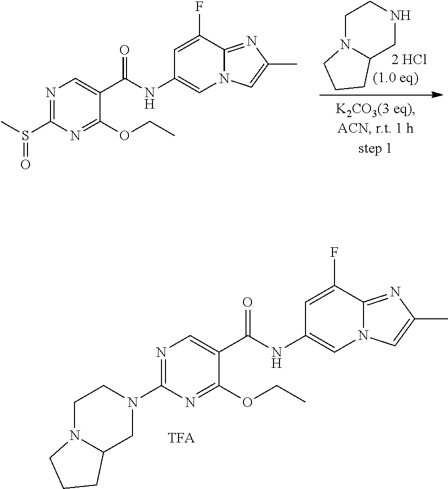

### Step 1: Preparation of 4-ethoxy-N-(8-fluoro-2-methylimidazo[1,2-a]pyridin-6-yl)-2-(hexahydropyrrolo[1,2-a]pyrazin-2 (1H)-yl)pyrimidine-5-carboxamide TFA salt

To a solution of 4-ethoxy-N-(8-fluoro-2-methylimidazo[1,2-a]pyridin-6-yl)-2-(methylsulfinyl)pyrimidine-5-carboxamide (54 mg, 0.143 mmol) in MeCN (4 mL) was added octahydropyrrolo[1,2-a]pyrazine dihydrochloride (33 mg, 0.172 mmol) and K₂CO₃ (60 mg, 0.429 mmol) at rt. The reaction mixture was stirred for 1 h at rt. After concentration, the residue was purified by pre-TLC (DCM:MeOH=20:1) and further purified by pre-HPLC (MeCN/0.05% TFA in water) to give the title product (25 mg, Y: 36%) as a yellow solid. ESI-MS (M+H)⁺: 440.1. ¹H NMR (400 MHZ, DMSO-d₆) δ 10.57, 9.89 (br s, 1H), 9.89 (s, 1H), 9.26 (s, 1H), 8.68 (s, 1H), 8.08 (s, 1H), 7.55 (d, J=11.9 Hz, 1H), 5.06-5.02 (m, 1H), 4.56 (q, J=7.0 Hz, 2H), 4.16 (br s, 2H), 3.52 (br s, 2H), 3.35 (br s, 1H), 3.25-2.96 (m, 3H), 2.41 (s, 3H), 2.19-2.15 (m, 2H), 1.90-1.86 (m, 2H), 1.43 (t, J=7.0 Hz, 3H).

### Example 6. 4-ethoxy-N-{8-fluoro-2-methylimidazo[1,2-a]pyridin-6-yl}-2-(4-methylpiperazin-1-yl)pyrimidine-5-carboxamide (Compound 6)

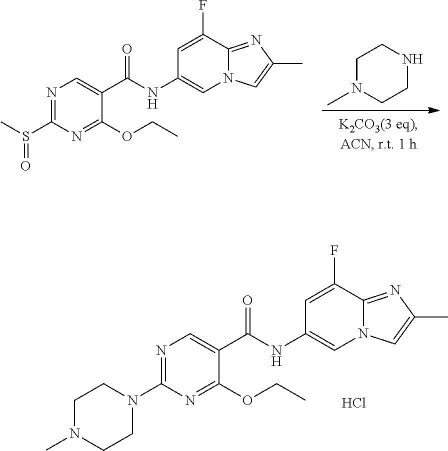

### Step 1: Preparation of 4-ethoxy-N-(8-fluoro-2-methylimidazo[1,2-a]pyridin-6-yl)-2-(4-methylpiperazin-1-yl)pyrimidine-S-carboxamide hydrochloride

To a solution of 4-ethoxy-N-(8-fluoro-2-methylimidazo[1,2-a]pyridin-6-yl)-2-(methylsulfinyl)pyrimidine-5-carboxamide (50 mg, 0.13 mmol) in ACN (5 mL) was added 1-methylpiperazine (13 mg, 0.13 mmol) and K₂CO₃ (53 mg, 0.39 mmol). The reaction mixture was stirred for 1 h at rt. The solid was filtered and the filtratrate was concentrated. The residue was purified by pre-HPLC (MeCN/0.05% HCl in water) to give title product (35 mg, Y: 60%) as a yellow solid. ESI-MS (M+H)⁺: 414.1.

¹H NMR (400 MHZ, DMSO)δ 11.11 (br s, 1H), 10.20 (s, 1H), 9.46 (s, 1H), 8.69 (s, 1H), 8.26 (s, 1H), 7.94 (d, J=10.7 Hz, 1H), 4.77 (d, J=14.0 Hz, 2H), 4.55 (q, J=7.0 Hz, 2H), 3.50-3.44 (m, 4H), 3.12-3.04 (m, 2H), 2.80 (d, J=4.2 Hz, 3H), 2.48 (s, 3H), 1.42 (t, J=7.0 Hz, 3H)

### Example 7. 4-ethoxy-N-{8-fluoro-2-methylimidazo[1,2-a]pyridin-6-yl}-2-[(3S)-3-methylpiperazin-1-yl]pyrimidine-5-carboxamide (Compound 7)

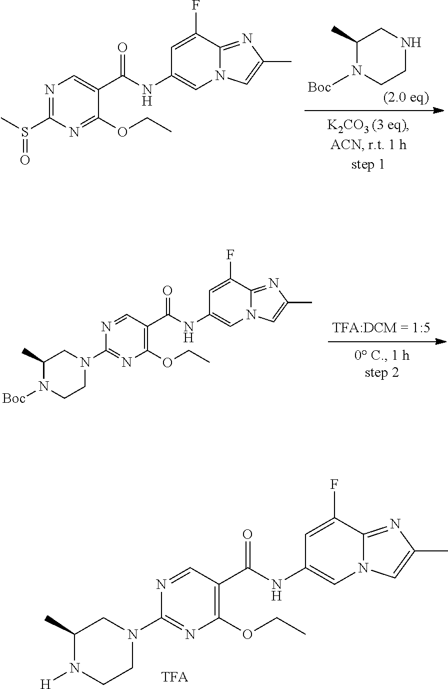

### Step 1: Preparation of ter-butyl(S)-4-(4-ethoxy-5-(8-fluoro-2-methylimidazo[1,2-a]pyridin-6-yl) carbamoyl)pyrimidin-2-yl)-2-methylpiperazine-1-carboxylate

To a solution of 4-ethoxy-N-(8-fluoro-2-methylimidazo[1,2-a]pyridin-6-yl)-2-(methylsulfinyl)pyrimidine-5-carboxamide (80 mg, 0.21 mmol) in ACN (5 mL) was added tert-butyl(S)-2-methylpiperazine-1-carboxylate (84 mg, 0.42 mmol) and K₂CO₃ (87 mg, 0.63 mmol). The reaction mixture was stirred for 1 h at rt. After concentration, the residue was purified by silica gel column chromatography (DCM:MeOH=20:1, v/v) to give title product (60 mg, Y: 56%) as a yellow solid. ESI-MS (M+H)⁺: 514.2. ¹H NMR (400 MHZ, CDCl₃)δ 9.14 (s, 1H), 9.02 (d, J=1.5 Hz, 1H), 8.91 (s, 1H), 7.33 (d, J=2.5 Hz, 1H), 6.50 (dd, J=10.9, 1.5 Hz, 1H), 4.70-4.46 (m, 4H), 4.31-4.29 (m, 1H), 3.89-3.87 (m, 1H), 3.24-3.22 (m, 1H), 3.15-2.98 (m, 2H), 2.40 (s, 3H), 1.52 (t, J=7.1 Hz, 3H), 1.42 (s, 9H), 1.07 (d, J=6.7 Hz, 3H).

### Step 2: Preparation of(S)-4-ethoxy-N-(8-fluoro-2-methylimidazo[1,2-a]pyridin-6-yl)-2-(3-methylpiperazin-1-yl)pyrimidine-5-carboxamide TFA salt

To a solution of tert-butyl(S)-4-(4-ethoxy-5-((8-fluoro-2-methylimidazo[1,2-a]pyridin-6-yl) carbamoyl)pyrimidin-2-yl)-2-methylpiperazine-1-carboxylate (55 mg, 0.11 mmol) in DCM (5 mL) was added TFA (1 mL) at 0° C. The reaction mixture was stirred at 0° C. for 1 h. The resulting mixture was diluted with DCM and concentrated to dryness and repeated three times. The residue was triturated with PE: EA=10:1 (5 ml), and filtered. The solid was dried under vacuum to give title product (22.48 mg, Y: 40%) as a yellow solid. ESI-MS (M+H)⁺: 414.4. ¹H NMR (400 MHz, DMSO-d₆) δ 9.85 (s, 1H), 9.23 (s, 2H), 8.90 (s, 1H), 8.67 (s, 1H), 8.05 (s, 1H), 7.50 (d, J=12.1 Hz, 1H), 4.68-4.65 (m, 2H), 4.55 (q, J=6.9 Hz, 2H), 3.45-3.33 (m, 3H), 3.17-3.14 (m, 2H), 2.40 (s, 3H), 1.43 (t. J=7.0 Hz, 3H), 1.28 (d, J=6.4 Hz, 3H).

### Example 8. 4-ethoxy-N-{8-fluoro-2-methylimidazo[1,2-a]pyridin-6-yl}-2-{octahydropyrrolo[3,4-c]pyrrol-2-yl}pyrimidine-5-carboxamide (Compound 8)

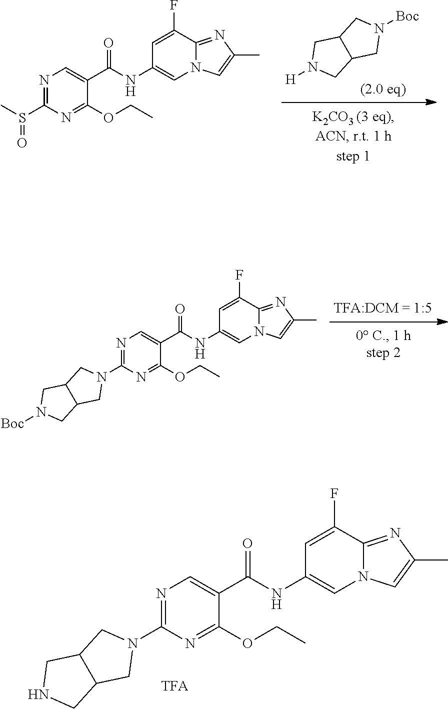

### Step 1: Preparation of ter-butyl 5-(4-ethoxy-5-((8-fluoro-2-methylimidazo[1,2-a]pyridin-6-yl) carbamoyl)pyrimidin-2-yl) hexahydropyrrolo[3,4-c]pyrrole-2 (1H)-carboxylate

To a solution of 4-ethoxy-N-(8-fluoro-2-methylimidazo[1,2-a]pyridin-6-yl)-2-(methylsulfinyl)pyrimidine-5-carboxamide (60 mg. 0.16 mmol) in MeCN (3 mL) was added tert-butyl hexahydropyrrolo[3,4-c]pyrrole-2 (1H)-carboxylate (67 mg, 0.32 mmol) and K₂CO₃ (66 mg, 0.48 mmol). The reaction mixture was stirred for 1 h at rt. After concentration, the residue was purified by silica gel column chromatography (DCM:MeOH=20:1, v/v) to give title product (50 mg, Y: 60%) as a yellow solid. ESI-MS (M+H)⁺: 526.2. ¹H NMR (400 MHZ, CDCl₃)δ 9.24 (s, 1H), 9.11 (s, 1H), 9.00 (s, 1H), 7.40 (d, J=2.S Hz, 1H), 6.57 (d, J=10.8 Hz, 1H), 4.63 (q, J=7.1 Hz, 2H), 3.89 (br s, 2H), 3.64 (br s, 2H), 3.34-3.30 (m, 2H), 3.01 (br s, 2H), 2.47 (s, 3H), 1.60-1.57 (m, 5H), 1.46 (s, 9H)

### Step 2: Preparation of 4-ethoxy-N-(8-fluoro-2-methylimidazo[1,2-a]pyridin-6-yl)-2-(hexahydropyrrolo[3,4-c]pyrrol-2 (1H)-yl)pyrimidine-5-carboxamide TFA salt

To a solution of tert-butyl 5-(4-ethoxy-5-((8-fluoro-2-methylimidazo[1,2-a]pyridin-6-yl) carbamoyl)pyrimidin-2-yl) hexahydropyrrolo[3,4-c]pyrrole-2 (1H)-carboxylate (40 mg, 0.076 mmol) in DCM (5 mL) was added TFA (1 mL) at 0° C. The reaction mixture was stirred at 0° C. for 1 h. The resulting mixture was diluted with DCM and concentrated to dryness and repeated three times. The residue was triturated with PE: EA=10:1 (5 mL) The solid was collected by filtrateion and dried under vacuum to give title product (7.82 mg, Y: 20%) as a yellow solid. ESI-MS (M+H)⁺: 426.4. ¹H NMR (400 MHZ, DMSO-d₆) δ 9.91 (s, 1H), 9.63-9.60 (m, 2H), 9.29 (s, 1H), 8.64 (s, 1H), 8.10 (s, 1H), 7.66 (d, J=12.1 Hz, 1H), 4.55 (q, J-7.0 Hz, 2H), 3.86-3.78 (m, 4H), 3.44-3.42 (m, 2H), 3.13 (br s, 4H), 2.42 (s, 3H), 1.43 (t, J=7.0 Hz, 3H).

### Example 9. 4-ethoxy-N-{2-methylimidazo[1,2-a]pyrazin-6-yl}-2-(piperazin-1-yl)pyrimidine-5-carboxamide (Compound 9)

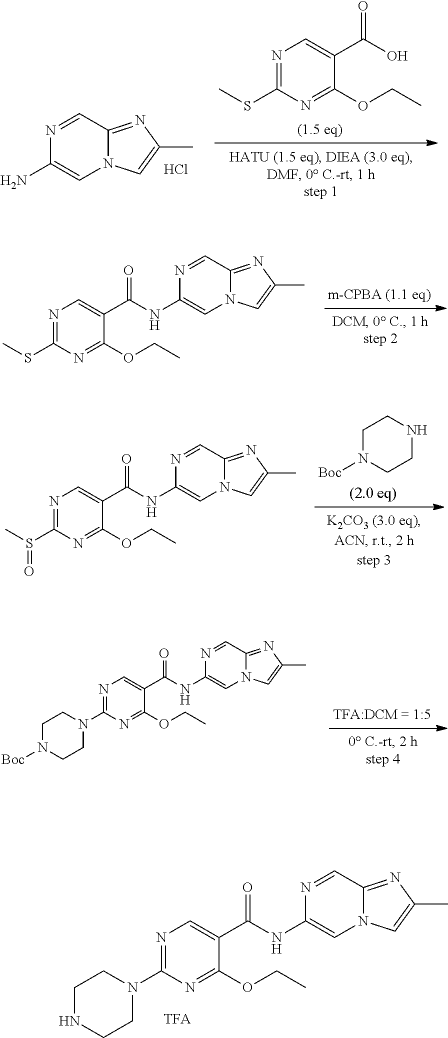

### Step 1: Preparation of 4-ethoxy-N-(2-methylimidazo[1,2-a]pyrazin-6-yl)-2-(methylthio)pyrimidine-5-carboxamide

To a mixture of 2-methylimidazo[1,2-a]pyrazin-6-amine hydrochloride (359 mg, 1.95 mmol) in DMF (10 ml) was added 4-ethoxy-2-(methylthio)pyrimidine-5-carboxylic acid (627 mg, 2.93 mmol), DIPEA (755 mg, 5.85 mmol) and HATU (1.1 g, 2.93 mmol) at 0° C. The mixture was stirred at rt for 1 h. After diluting with water (10 mL), the mixture was filtered and the cake was washed with EtOAc (5 mL), dried under vacuum to give title product (160 mg, Y: 24%) as a yellow solid. ESI-MS (M+H)⁺: 345.1. ¹H NMR (400 MHZ, CDCl₃)δ 9.88 (s, 1H), 9.27 (d, J=1.4 Hz, 1H), 9.14 (s, 1H), 8.75 (d, J=0.7 Hz, 1H), 7.50 (s. 1H), 4.75 (q, J=7.1 Hz, 2H), 2.61 (s, 3H), 2.52 (s, 3H), 1.62 (t, J=7.1 Hz, 3H).

### Step 2: Preparation of 4-ethoxy-N-(2-methylimidazo[1,2-a]pyrazin-6-yl)-2-(methylsulfinyl)pyrimidine-5-carboxamide

To a mixture of 4-ethoxy-N-(2-methylimidazo[1,2-a]pyrazin-6-yl)-2-(methylthio)pyrimidine-5-carboxamide (155 mg, 0.45 mmol) in DCM (5 mL) was added m-CPBA (86 mg, 0.50 mmol) at 0° C. The reaction mixture was stirred at 0° C. for 1 h and quenched with aqueous saturated Na₂S₂O₃. The mixture was extracted with DCM (10 mL×3). The combined organic layers was dried over sodium sulfate and concentrated. The crude title product (150 mg, Y: 93%) was obtained as a yellow solid, which was used in the next step without further purification. ESI-MS (M+H)⁺: 361.0.

### Step 3: Preparation of tert-butyl 4-(4-ethoxy-5-((2-methylimidazo[ 1,2-a]pyrazin-6-yl) carbamoyl)pyrimidin-2-yl) piperazine-1-carboxylate

To a solution of 4-ethoxy-N-(2-methylimidazo[1,2-a]pyrazin-6-yl)-2-(methylsulfinyl)pyrimidine-5-carboxamide (50 mg, 0.14 mmol) in MeCN (3 mL) was added tert-butyl piperazine-1-carboxylate (52 mg, 0.28 mmol) and K₂CO₃ (58 mg, 0.42 mmol) at rt. The reaction mixture was stirred for 2 h at rt. After concentration, the residue was purified by silica gel column chromatography (DCM:MeOH=20:1) to give title product (35 mg. Y: 52%) as a yellow solid. ESI-MS (M+H)⁺: 483.2. ¹H NMR (400 MHZ, CDCl₃)δ 9.81 (s, 1H), 9.27 (d, J=1.4 Hz, 1H), 9.00 (s, 1H), 8.74 (s, 1H), 7.47 (s, 1H), 4.64 (q, J=7.1 Hz, 2H), 3.90 (br s, 4H), 3.57-3.48 (m, 4H), 2.51 (s, 3H), 1.59 (t, J=7.1 Hz, 3H), 1.50 (s, 9H).

### Step 4: Preparation of 4-ethoxy-N-(2-methylimidazo[1,2-a]pyrazin-6-yl)-2-(piperazin-1-yl)pyrimidine-5-carboxamide TFA salt

To a solution of tert-butyl 4-(4-ethoxy-5-((2-methylimidazo[1,2-a]pyrazin-6-yl) carbamoyl)pyrimidin-2-yl) piperazine-1-carboxylate (35 mg, 0.073 mmol) in DCM (10 mL) was added TFA (2 mL) at 0° C. The reaction mixture was stirred at rt for 2 h. The resulting mixture was diluted with DCM and concentrated to dryness. The residue was treated with PE: EA (10:1) and the precipitate was filtered. The solid was washed with PE and dried under vacuum to give title product (19.59 mg, Y: 56%) as a yellow solid. ESI-MS (M+H)⁺: 383.2.1H NMR (400 MHZ, DMSO-d₆) δ 9.91 (s, 1H), 9.36 (d, J=1.4 Hz, 1H), 8.98 (br s, 2H), 8.87 (s, 1H), 8.80 (s, 1H), 8.08 (s, 1H), 4.59 (t, J=7.1 Hz, 2H), 4.08-4.00 (m, 4H), 3.23 (br s, 4H), 2.42 (s, 3H), 1.48 (t, J=7.1 Hz, 3H).

### Example 10. 2-(3,3-dimethylpiperazin-1-yl)-4-ethoxy-N-{2-methylimidazo[1,2-a]pyrazin-6-yl}pyrimidine-5-carboxamide (Compound 10)

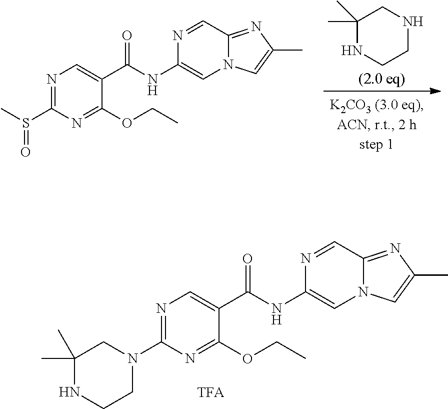

### Step 1: Preparation of 2-(3,3-dimethylpiperazin-1-yl)-4-ethoxy-N-(2-methylimidazo[1,2-a]pyrazin-6-yl)pyrimidine-5-carboxamide

To a solution of 4-ethoxy-N-(2-methylimidazo[1,2-a]pyrazin-6-yl)-2-(methylsulfinyl)pyrimidine-5-carboxamide (50 mg, 0.14 mmol) in MeCN (3 mL) was added 2,2-dimethylpiperazine (32 mg, 0.28 mmol) and K₂CO₃ (58 mg, 0.42 mmol). The reaction mixture was stirred for 2 h at rt. After concentration, the residue was purified by silica gel column chromatography (DCM:MeOH=10:1, v/v) to give title product as a free base. TFA salt was obtained after treated with DCM/TFA system to give title product (37 mg, Y: 49%) as a yellow solid. ESI-MS (M+H)⁺: 411.1. ¹H NMR (400 MHZ, DMSO-d₆) δ 9.91 (s, 1H), 9.36 (d, J=1.2 Hz, 1H), 9.04 (s, 2H), 8.86 (s, 1H), 8.79 (s, 1H), 8.08 (s, 1H), 4.60 (q, J=6.9 Hz, 2H), 4.05 (br s, 2H), 3.90 (s, 2H), 3.28 (br s, 2H), 2.42 (s, 3H), 1.48 (t, J=7.0 Hz, 3H), 1.32 (s, 6H).

### Example 11. 4-ethoxy-2-(3-ethylpiperazin-1-yl)-N-{8-fluoro-2-methylimidazo[1,2-a]pyridin-6-yl}pyrimidine-5-carboxamide (Compound 11)

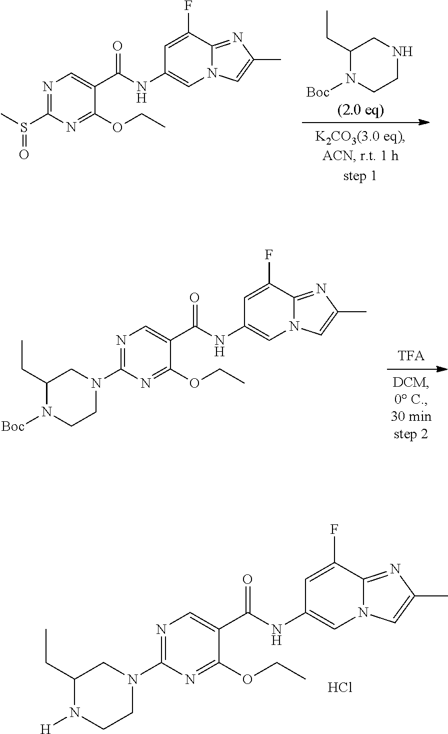

### Step 1: Preparation of tert-butyl 4-(4-ethoxy-5-((8-fluoro-2-methylimidazo[1,2-a]pyridin-6-yl) carbamoyl)pyrimidin-2-yl)-2-ethylpiperazine-1-carboxylate

To a solution of 4-ethoxy-N-(8-fluoro-2-methylimidazo[1,2-a]pyridin-6-yl)-2-(methylsulfinyl)pyrimidine-5-carboxamide (80 mg, 0.212 mmol) in MeCN (8 mL) was added tert-butyl 2-ethylpiperazine-1-carboxylate (55 mg, 0.255 mmol) and K₂CO₃ (59 mg, 0.424 mmol). The reaction mixture was stirred for 1 h at rt. After concentration, the residue was purified by silica gel column chromatography (EA: PE=1:5) to give title product (50 mg, Y: 44%) as a yellow solid. ESI-MS (M+H)⁺: 528.3.

### Step 2: Preparation of 4-ethoxy-2-(3-ethylpiperazin-1-yl)-N-(8-fluoro-2-methylimidazo[1,2-a]pyridin-6-yl)pyrimidine-5-carboxamide TFA salt

To a solution of tert-butyl 4-(4-ethoxy-5-((8-fluoro-2-methylimidazo[1,2-a]pyridin-6-yl) carbamoyl)pyrimidin-2-yl)-2-ethylpiperazine-1-carboxylate (50 mg, 0.088 mmol) in DCM (5 mL) was added TFA (1 mL) at 0° C. The reaction mixture was stirred at rt for 1 h. After concentration, the residue was purified by pre-HPLC (MeCN/0.05% TFA in water) to give title product (13.6 mg, Y: 33%) as a yellow solid. ESI-MS (M+H)⁺: 428.2. ¹H NMR (400 MHz, DMSO-d₆) δ 9.82 (s, 1H), 9.21 (s, 1H), 9.06 (s, 1H), 8.86 (s, 1H), 8.68 (s. 1H), 8.03 (s, 1H), 7.47 (d, J=12.0 Hz, 1H), 4.75-4.61 (m, 2H), 4.58-4.49 (m, 2H), 3.48-3.36 (m, 2H), 3.19-3.10 (m, 3H), 2.39 (s, 3H), 1.70-1.60 (m, 2H), 1.43 (t, J=7.0 Hz, 3H), 1.00 (t, J=7.5 Hz, 3H).

### Example 12. 4-ethoxy-N-(4-fluoro-2-methyl-1,3-benzoxazol-6-yl)-2-(piperazin-1-yl)pyrimidine-5-carboxamide (Compound 12)

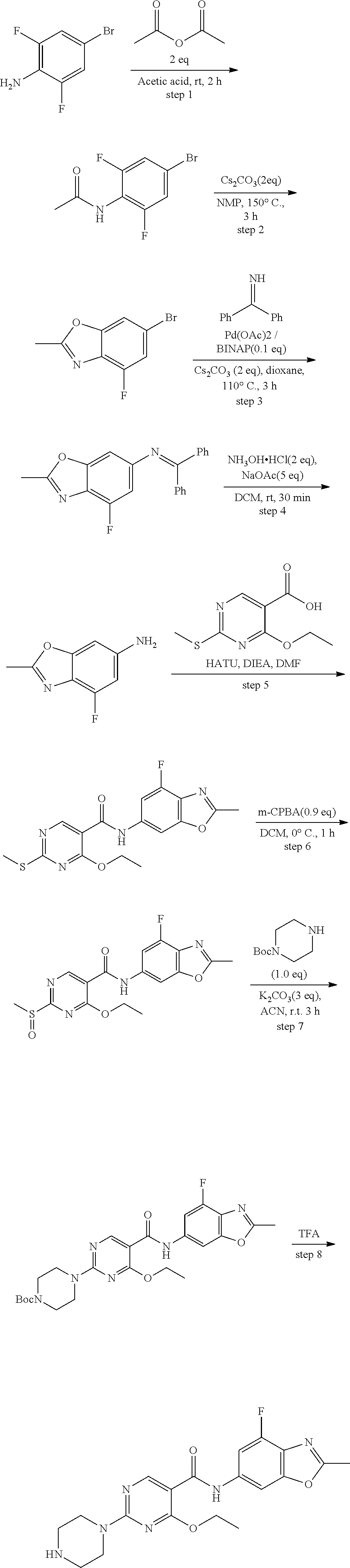

### Step 1: synthesis of N-(4-bromo-2,6-difluorophenyl) acetamide

To a mixture of 4-bromo-2,6-difluoroaniline (5 g, 24.1 mmol) in AcOH (20 mL) was added and acetic anhydride (15 mL) at 0° C., and the reaction mixture was stirred for 2 h at 0° C. The mixture was diluted with water (250 mL). The precipitate was collected and washed with water (50 mL) and dried to give the desired product as white solid (4.5 g, crude yield: 85%), which was used to next step without further purification. ESI-MS: [M+H]⁺250.0

### Step 2: synthesis of 6-bromo-4-fluoro-2-methylbenzo[d]oxazole

A suspension of N-(4-bromo-2,6-difluorophenyl) acetamide (3 g, 12.0 mmol) and Cs₂CO₃ (7.8 g, 24.0 mmol) in NMP (50 mL) was stirred for 3 h at 150° C. After cooling to rt, the mixture was diluted with water (250 mL) and extracted with EA (150 mL×2). The organic layer was washed with brine, dried over Na₂SO₄ and concentrated to dryness. The crude was purified by silica gel column (10-50% EA in PE). The product was obtained as colorless oil (1.2 g, Y: 44%). ESI-MS: [M+H]⁺230.1

### Step 3: synthesis of N-(4-fluoro-2-methylbenzo[d]oxazol-6-yl)-1,1-diphenylmethanimine

A mixture of 6-bromo-4-fluoro-2-methylbenzo[d]oxazole (1.2 g, 5.02 mmol), diphenylmethanimine (1.3 g, 7.50 mmol), Pd₂(dba)₃ (240 mg, 0.25 mmol), BINAP (150 mg, 0.25 mmol) and Cs₂CO₃ (5.0 g, 15.1 mmol) in tolune (25 mL) at room temperature was purged with N₂ for three times. Then the reaction mixture was stirred at 100° C. for 16 h. After cooling to rt, the mixture was diluted with water and extracted with EA. The EA layer was washed with brine, dried over anhylous Na₂SO₄ and concentrated to dryness. The crude was purified by silica gel column (10˜100% EA in PE). The product was obtained as yellow oil (500 mg, yield: 35%.) ESI-MS: [M+H]⁺331.1

### Step 4: synthesis of +-fluoro-2-methylbenzo[d]oxazol-6-amine

A mixture of N-(4-fluoro-2-methylbenzo[d]oxazol-6-yl)-1,1-diphenylmethanimine (500 mg, 1.6 mmol), NH₂OH.HCl (260 mg, 3.2 mmol) and NaOAc (600 mg, 8.1 mmol) in MeOH (50 ml) was stirred for 1 h at rt. The mixture was concentrated under reduced pressure. The residue was diluted with water (50 mL), extracted with EA (50 ml×2). The combined organics was washed with brine, dried over anhylous Na₂SO₄, filtered and concentrated. The crude product (250 mg, crude yield: 93%)) as yellow oil was used to next step without further purification. ESI-MS: [M+H]⁺166.9.

### Step 5: synthesis of 4-ethoxy-N-(4-fluoro-2-methylbenzo[d]oxazol-6-yl)-2-(methylthio)pyrimidine-5-carboxamide

To a solution of 4-fluoro-2-methylbenzo[d]oxazol-6-amine (250 mg, 1.5 mmol) in DMF (10 mL) was added 4-ethoxy-2-(methylthio)pyrimidine-5-carboxylic acid (400 mg, 1.8 mmol), HATU (680 mg, 1.8 mmol) and DIEA (590 mg, 4.5 mmol). The reaction mixture was stirred for 1 h at rt. Water (50 mL) was added, and the mixture was filtered to give a filter cake, the crude was purified by silica gel column (10˜100% EA in PE). The product was obtained as yellow solid (160 mg, yield: 30%). ESI-MS: [M+H]⁺363.1

### Step 6: synthesis of 4-ethoxy-N-(4-fluoro-2-methylbenzo[d]oxazol-6-yl)-2-(methylsulfinyl)pyrimidine-5-carboxamide

To a mixture of 4-ethoxy-N-(4-fluoro-2-methylbenzo[d]oxazol-6-yl)-2-(methylthio)pyrimidine-5-carboxamide (30 mg, 0.08 mmol) in DCM (5 mL) was added m-CPBA (16 mg, 0.09 mmol) at 0° C., and the mixture was stirred for 30 min at rt. The reaction mixture was diluted with water (15 mL), extracted with DCM (20 mL×2). The combined organics were washed with brine, dried over sodium sulfate. After concentration under reduced pressure, the crude title product was obtained as yellow solid (30 mg, crude yield: 94%), which was used to next step directly.

### ESI-MS: [M+H]⁺379.1

### Step 7: synthesis of tert-butyl 4-(4-ethoxy-5-((44-fluoro-2-methylbenzo[d]oxazol-6-yl) carbamoyl)pyrimidin-2-yl) piperazine-1-carboxylate

A stirred solution of 4-ethoxy-N-(4-fluoro-2-methylbenzo[d]oxazol-6-yl)-2-(methylsulfinyl)pyrimidine-5-carboxamide (30 mg, 0.08 mmol) and tert-butyl piperazine-1-carboxylate (19 mg, 0.1 mmol), K₂CO₃ (33 mg, 0.24 mmol) in ACN (5 mL) was stirred at rt for 3 h. After filtration, the filtrate was concentrated to dryness and the residue was diluted with water, extreated with EA. The origanic phase was washed with brine, dried with anhydrous Na₂SO₄, filtered and concentrated to give title product as a yellow solid (30 mg, crude, yield: 75%), which was used to next step directly. ESI-MS: [M+H]⁺501.2

### Step 8: synthesis of 4-ethoxy-N-(4-fluoro-2-methylbenzo[d]oxazol-6-yl)-2-(piperazin-1-yl)pyrimidine-5-carboxamide

To a solution of tert-butyl 4-(4-ethoxy-5-((4-fluoro-2-methylbenzo[d]oxazol-6-yl) carbamoyl)pyrimidin-2-yl) piperazine-1-carboxylate (30 mg, 0.06 mmol) in DCM (5 ml) was added TFA (0.5 mL) at 0° C. The reaction mixture was warmed to rt and stirred for 1 h. After concentration, the residue was purified by pre-HPLC to give title product as white solid (5 mg, yield: 20%). ESI-MS: [M+H]⁺401.0. ¹H NMR (400 MHZ, MeOD-d₄)δ 8.83 (d, J=1.5 Hz, 1H), 7.93 (s, 1H), 7.39-7.35 (m, 1H), 4.65 (d, J=7.1 Hz, 2H), 4.19-4.15 (m, 4H), 3.33-3.30 (m, 4H), 2.64 (s, 3H), 1.55 (t. J=7.1 Hz, 3H).

### Example 14. 4-ethoxy-N-(4-fluoro-2-methyl-1,3-benzoxazol-6-yl)-2-{octahydropyrrolo[3,4-c]pyrrol-2-yl}pyrimidine-5-carboxamide (Compound 14)

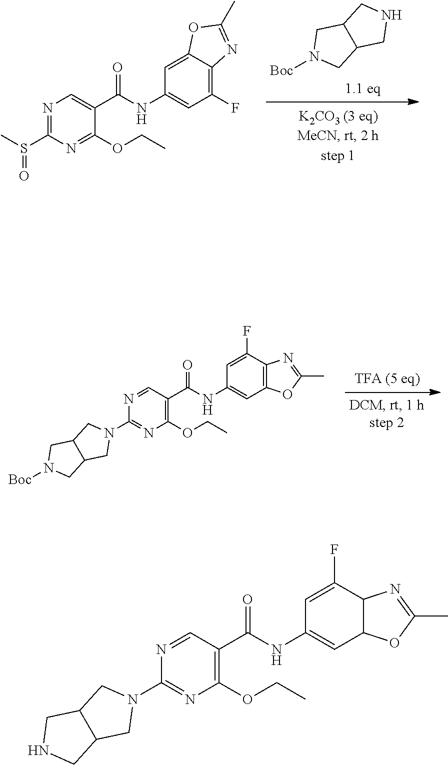 [SMILES: CCOc1nc(S(C)=O)ncc1C(=O)Nc1cc(F)c2nc(C)oc2c1 ; CC(C)(C)OC(=O)N1CC2CNCC2C1 ; CCOc1nc(N2CC3CN(C(=O)OC(C)(C)C)CC3C2)ncc1C(=O)Nc1cc(F)c2nc(C)oc2c1 ; CCOc1nc(N2CC3CNCC3C2)ncc1C(=O)NC1=CC2OC(C)=NC2C(F)=C1]

### Step 1: synthesis of ter-butyl 5-(4-ethoxy-5-(4-fluoro-2-methylbenzo[d]oxazol-6-yl) carbamoyl)pyrimidin-2-yl) hexahydropyrrolo[3,4-c]pyrrole-2 (1H)-carboxylate

To a suspension of 4-ethoxy-N-(4-fluoro-2-methylbenzo[d]oxazol-6-yl)-2-(methylsulfinyl)pyrimidine-5-carboxamide (60 mg, 0.158 mmol) and tert-butyl hexahydropyrrolo[3,4-c]pyrrole-2 (1H)-carboxylate (35 mg, 0.190 mmol) in MeCN (2 mL) was added K₂CO₃ (65 mg, 0.594 mmol), and the reaction mixture was stirred at rt for 2 h. The mixture was diluted with water and extracted with EA. The EA layer was washed with brine, dried over Na₂SO₄ and concentrated to dryness. The crude was purified by silica gel column chromatography (10˜50% EA in PE). The product was obtained as off-white solid (50 mg, yield: 40%). MS m/z (ESI): [M+H]⁺ 527.3. ¹H NMR (400 MHZ, DMSO-d₆) δ 9.85 (s, 1H), 8.63 (s, 1H), 8.00 (d, J=1.5 Hz, 1H), 7.51 (dd, J=12.2, 1.6 Hz, 1H), 4.54 (q, J=7.0 Hz, 2H), 3.80 (s, 2H), 3.59-3.42 (m, 4H), 3.19-3.13 (m, 2H), 2.99 (s, 2H), 2.61 (S, 3H), 1.44 (t, J=7.0 Hz, 3H), 1.39 (s, 9H).

### Step 2: synthesis of 4-ethoxy-N-(4-fluoro-2-methylbenzo[d]oxazol-6-yl)-2-(hexahydropyrrolo[3,4-c]pyrrol-2 (1H)-yl)pyrimidine-5-carboxamide

To a solution of tert-butyl 5-(4-ethoxy-5-((4-fluoro-2-methylbenzo[d]oxazol-6-yl) carbamoyl)pyrimidin-2-yl) hexahydropyrrolo[3,4-c]pyrrole-2 (1H)-carboxylate (50 mg, 0.095 mmol) in DCM (S mL) was added TFA (0.5 mL) at 0° C. After addition, the reaction was stirred for 1 h at rt. After concentration, the crude was purified by pre-HPLC to provide title product as white solid (10 mg, yield: 25%). ¹H NMR (400 MHZ, DMSO-d₆) & 9.22 (d, J=1.4 Hz, 1H), 8.31 (s, 2H), 7.93 (d, J=2.4 Hz, 1H), 7.87-7.79 (m, 3H), 7.09 (s, 1H), 3.61 (s, 4H), 2.89 (s, 4H), 2.38 (s, 3H). MS m/z (ESI): [M+H]⁺427.2. ¹H NMR (400 MHZ, DMSO)δ 9.86 (s, 1H), 8.64 (s, 1H), 8.00 (s, 1H), 7.52 (d, J=12.4 Hz, 1H), 4.56-4.54 (m, 2H), 3.87-3.77 (m, 2H), 3.54 (d, J=9.2 Hz, 2H), 3.26-3.20 (m, 2H), 3.03 (s, 2H), 2.96 (d, J=10.9 Hz, 2H), 2.61 (s, 3H), 1.44 (t, J=7.0 Hz, 3H).

### Example 15. 2-(1,4-diazepan-1-yl)-4-ethoxy-N-{8-fluoro-2-methylimidazo[1,2-a]pyridin-6-yl}pyrimidine-5-carboxamide (Compound 15)

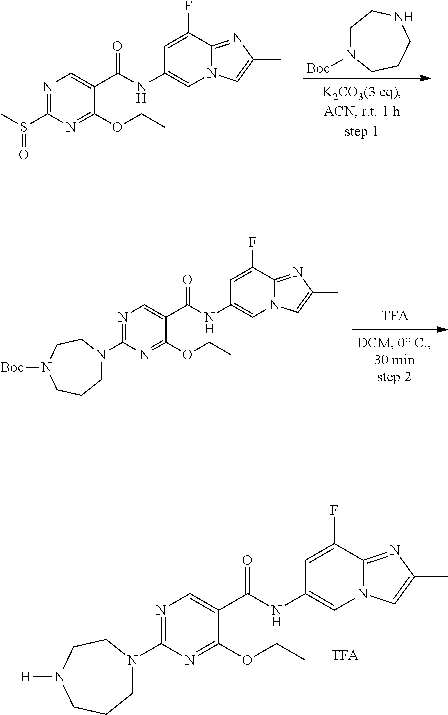

### Step 1: tert-butyl 4-(4-ethoxy-5-((8-fluoro-2-methylimidazo[1,2-a]pyridin-6-yl) carbamoyl)pyrimidin-2-yl)-1,4-diazepane-1-carboxylate

To a solution of 4-ethoxy-N-(8-fluoro-2-methylimidazo[1,2-a]pyridin-6-yl)-2-(methylsulfinyl)pyrimidine-5-carboxamide (90 mg, 0.24 mmol) in ACN (5 mL) was added tert-butyl 1,4-diazepane-1-carboxylate (48 mg, 0.24 mmol) and K₂CO₃ (99 mg, 0.72 mmol). The reaction mixture was stirred for 3 h at rt. After concentration, the residue was purified by silica gel column chromatography (DCM: MeOH-20:1) to give title product (60 mg, Y: 48%) as a yellow solid. ESI-MS (M+H)⁺: 514.1.

### Step 2:2-(1,4-diazepan-1-yl)-4-ethoxy-N-(8-fluoro-2-methylimidazo[1,2-a]pyridin-6-yl)pyrimidine-5-carboxamide 2,2,2-trifluoroacetate

To a solution of tert-butyl 4-(4-ethoxy-5-((8-fluoro-2-methylimidazo[1,2-a]pyridin-6-yl) carbamoyl)pyrimidin-2-yl)-1,4-diazepane-1-carboxylate (51 mg, 0.1 mmol) in DCM (3 mL) was TFA (1 mL) at 0° C. The reaction mixture was stirred 30 min. After concentration, the residue was purified by pre-HPLC (MeCN/0.05% TFA in water) to give title product (11 mg, Y: 21%) as a yellow solid. ESI-MS (M+H)⁺: 414.0. ¹H NMR (400 MHZ, DMSO)δ 9.77 (s, 1H), 9.22 (s, 1H), 8.80-8.58 (m, 3H), 8.03 (s, 1H), 7.49 (s, 1H), 4.55 (q, J=7.0 Hz, 2H), 4.03 (br s, 2H), 3.89 (d, J=6.2 Hz, 2H), 3.31 (s, 3H), 3.21 (br s, 2H), 2.39 (br s, 2H), 2.05 (br s, 2H), 1.43 (t, J=7.0 Hz, 3H).

### Example 16. N-{2,8-dimethylimidazo[1,2-a]pyrazin-6-yl}-4-ethoxy-2-(piperazin-1-yl)pyrimidine-5-carboxamide (Compound 16)

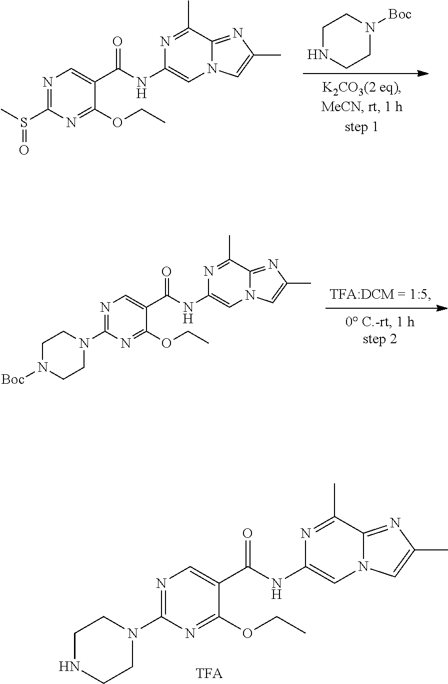

### Step 1: synthesis of tert-butyl 4-(5-((2,8-dimethylimidazo[1,2-a]pyrazin-6-yl) carbamoyl)-4-ethoxypyrimidin-2-yl) piperazine-1-carboxylate

A mixture of N-(2,8-dimethylimidazo[1,2-a]pyrazin-6-yl)-4-ethoxy-2-(methylsulfinyl)pyrimidine-5-carboxamide (80 mg, 0.21 mmol), tert-butyl piperazine-1-carboxylate (59 mg, 0.32 mmol) and K₂CO₃ (58 mg, 0.42 mmol), in MeCN (3 mL) was stirred at rt for 1 h. The solid was filtered off and the filtrate was concentrated. The residue was purified by silica gel column chromatography (1˜10% MeOH in DCM). The product was afforded as white solid crude (95 mg, yield: 90%). MS m/z (ESI): [M+H]⁺497.5

### Example 17. 2-(2,7-diazaspiro[3.5]nonan-2-yl}-N-{2,8-dimethylimidazo[1,2-a]pyrazin-6-yl;-4-ethoxypyrimidine-5-carboxamide (Compound 17)

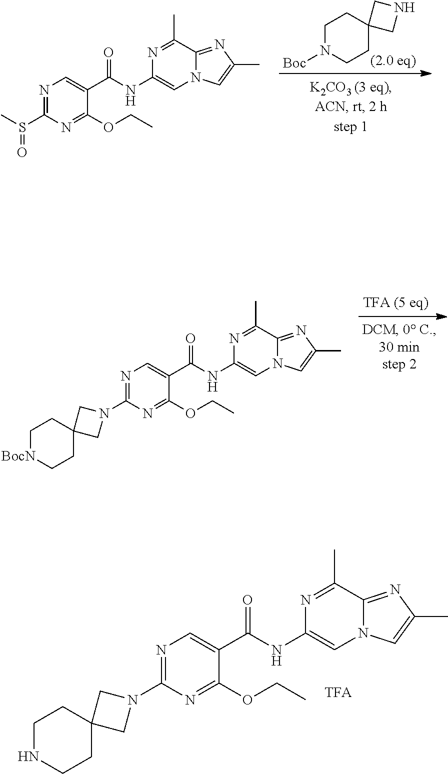

### Step 1: Preparation of tert-butyl 2-(5-((2,8-dimethylimidazo {1,2-a}pyrazin-6-yl) carbamoyl)-4-ethoxypyrimidin-2-yl)-2,7-diazaspiro[3.5]nonane-7-carboxylate

To a solution of N-(2,8-dimethylimidazo[1,2-a]pyrazin-6-yl)-4-ethoxy-2-(methylsulfinyl)pyrimidine-5-carboxamide (80 mg, 0.212 mmol) in ACN (8 mL) was added tert-butyl 2,7-diazaspiro[3.5]nonane-7-carboxylate (55 mg, 0.255 mmol) and K₂CO₃ (59 mg, 0.424 mmol). The reaction mixture was stirred for 1 h at rt. After concentration, the residue was purified by silica gel column chromatography (EA: PE=1:5) to give title product (50 mg, Y: 44%) as a yellow solid. ESI-MS (M+H)⁺: 528.3. ¹H NMR (400 MHZ, DMSO-d₆)δ 9.75 (s, 1H), 9.14 (s, 1H), 8.72 (s, 1H), 7.96 (s, 1H), 4.56 (d, J=7.1 Hz, 2H), 3.87 (s, 4H), 3.22 (br s, 4H), 2.90 (s, 3H), 2.68 (s, 3H), 1.70 (br s, 4H), 1.47 (t, J=7.0 Hz, 3H), 1.40 (s, 9H).

### Step 2: Preparation of N-(2,8-dimethylimidazo[1,2-a]pyrazin-6-yl)-4-ethoxy-2-(2,7-diazaspiro[3.5]nonan-2-yl)pyrimidine-5-carboxamide TFA salt

To a solution of tert-butyl 2-(5-((2,8-dimethylimidazo[1,2-a]pyrazin-6-yl) carbamoyl)-4-ethoxypyrimidin-2-yl)-2,7-diazaspiro[3.5]nonane-7-carboxylate (53 mg, 0.1 mmol) in DCM (10 mL) was added TFA (1 mL) at 0° C. The reaction mixture was stirred at rt for 1 h. After concentration, the residue was purified by pre-HPLC (MeCN/0.05% TFA in water) to give title product (45 mg, Y: 80%) as a yellow solid. ESI-MS (M+H): 437.0. ¹H NMR (400 MHZ, DMSO-d₆) δ 9.85 (s, 1H), 9.23 (s, 1H), 8.72 (s, 1H), 8.52 (br s, 2H), 8.06 (s, 1H), 4.56 (q, J=7.1 Hz, 2H), 3.93 (s, 4H), 3.09 (s, 4H), 2.70 (s, 3H), 2.42 (s, 3H), 1.95 (s, 4H), 1.47 (t, J=7.1 Hz, 3H).

### Example 18. 2-{2,6-diazaspiro[3.3]heptan-2-yl}-N-{2,8-dimethylimidazo[1,2-a]pyrazin-6-yl}-4-ethoxypyrimidine-5-carboxamide (Compound 18)

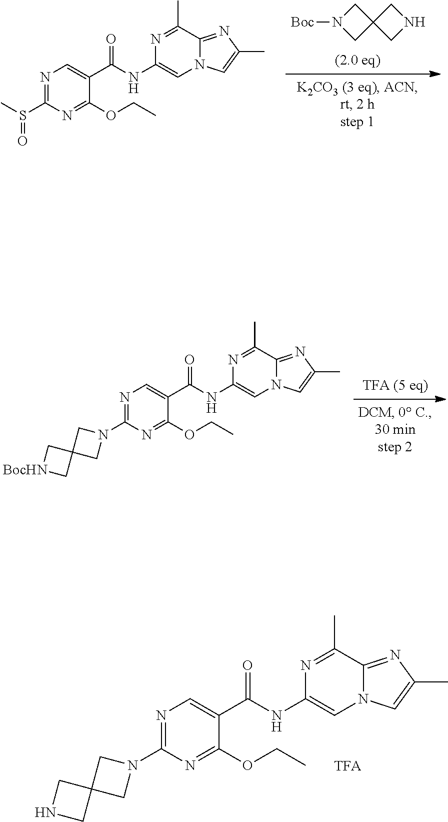

### Step 1: Preparation of tert-butyl 6-(5-((2,8-dimethylimidazo[1,2-a]pyrazin-6-yl) carbamoyl)-4-ethoxypyrimidin-2-yl)-2,6-diazaspiro[3.3]heptane-2-carboxylate

To a solution of N-(2,8-dimethylimidazo[1,2-a]pyrazin-6-yl)-4-ethoxy-2-(methylsulfinyl)pyrimidine-5-carboxamide (70 mg, 0.168 mmol) in ACN (3 mL) was added tert-butyl 2,6-diazaspiro[3.3]heptane-2-carboxylate (97 mg, 0.335 mmol) and K₂CO₃ (70 mg, 0.504 mmol). The reaction mixture was stirred for 2 h at rt. After concentration, the residue was purified by silica gel column chromatography (EA: PE=1:5) to give title product (55 mg, Y: 65%) as a light yellow solid. ESI-MS (M+H)⁺: 509.2. ¹H NMR (400 MHZ, DMSO-d₆) δ 9.75 (s, 1H), 9.14 (s, 1H), 8.71 (s, 1H), 7.95 (s, 1H), 4.58-4.52 (m, 2H), 4.26 (s, 4H), 3.63 (s, 4H), 2.37 (s, 3H), 2.18 (s, 3H), 1.47 (t, J=7.0 Hz, 3H), 1.38 (s, 9H).

### Step 2: Preparation of N-(2,8-dimethylimidazo[1,2-a]pyrazin-6-yl)-4-ethoxy-2-(2,6-diazaspiro[3.3]heptan-2-yl) pyramidine-5-carboxamide

To a solution of tert-butyl 6-(5-((2,8-dimethylimidazo[1,2-a]pyrazin-6-yl) carbamoyl)-4-ethoxypyrimidin-2-yl)-2,6-diazaspiro[3.3]heptane-2-carboxylate (53 mg. 0.1 mmol) in DCM (10 mL) was added TFA (1 mL) at 0° C. The reaction mixture was stirred at rt for 1 h. After concentration, the residue was purified by pre-HPLC (MeCN/0.05% TFA in water) to give title product (13.5 mg, Y: 20.7%) as a yellow solid. ESI-MS (M+H)⁺: 409.2. ¹H NMR (400 MHZ, DMSO-d₆) δ 9.86 (s, 1H), 9.22 (s, 1H), 8.72 (s, 1H), 8.63 (br s, 2H), 8.05 (s, 1H), 4.56 (q, J=6.9 Hz, 2H), 4.31 (s, 4H), 4.19 (t, J=5.9 Hz, 4H), 2.70 (s, 3H), 2.42 (s. 3H), 1.47 (t, J=7.0 Hz, 3H).

### Example 19. N-{2,8-dimethylimidazo[1,2-a]pyrazin-6-yl}-4-ethoxy-2-[(3S)-3-methylpiperazin-1-yl]pyrimidine-5-carboxamide (Compound 19)

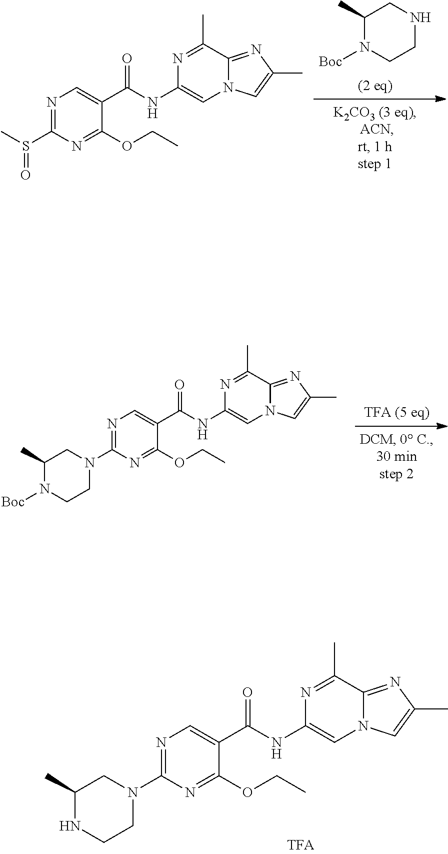

### Step 1: Preparation of teri-butyl(S)-4-(5-((2,8-dimethylimidazo[1,2-a]pyrazin-6-yl) carbamoyl)-4-ethoxypyrimidin-2-yl)-2-methylpiperazine-1-carboxylate

To a solution of N-(2,8-dimethylimidazo[1,2-a]pyrazin-6-yl)-4-ethoxy-2-(methylsulfinyl)pyrimidine-5-carboxamide (80 mg, 0.212 mmol) in ACN (8 mL) was added tert-butyl(S)-2-methylpiperazine-1-carboxylate (85 mg, 0.424 mmol) and K₂CO₃ (59 mg, 0.424 mmol). The reaction mixture was stirred for 1 h at rt. After concentration, the residue was purified by silica gel column chromatography (EA: PE==1:5) to give title product (75 mg, Y: 69%) as a yellow solid. ESI-MS (M+H)⁺: 511.3.

### Step 2: Preparation of(S)—N-(2,8-dimethylimidazo[1,2-a]pyrazin-6-yl)-4-ethoxy-2-(3-methylpiperazin-1-yl)pyrimidine-5-carboxamide TFA salt

To a solution of tert-butyl(S)-4-(5-((2,8-dimethylimidazo[1,2-a]pyrazin-6-yl) carbamoyl)-4-ethoxypyrimidin-2-yl)-2-methylpiperazine-1-carboxylate (75 mg, 0.14 mmol) in DCM (5 mL) was added TFA (1 mL) at 0° C. The reaction mixture was stirred at rt for 1 h. After concentration, the residue was purified by pre-HPLC (MeCN/0.05% TFA in water) to give title product (52 mg, Y: 72%) as a yellow solid. ESI-MS (M+H)⁺: 411.2. ¹H NMR (400 MHZ, DMSO)δ 9.90 (s, 1H), 9.23 (s, 2H), 8.90 (s, 1H), 8.78 (s, 1H), 8.06 (s, 1H), 4.68 (d, J=12.8 Hz, 2H), 4.60 (q, J=7.0 Hz, 2H), 3.40-3.36 (m, 3H), 3.24-3.04 (m, 2H), 2.71 (s, 3H), 2.42 (s, 3H), 1.48 (t, J=7.0 Hz, 3H), 1.29 (d, J=6.4 Hz, 3H).

### Example 20. N-{2,8-dimethylimidazo[1,2-a]pyrazin-6-yl}-4-ethoxy-2-{octahydropyrrolo[3,4-c]pyrrol-2-yl}pyrimidine-5-carboxamide (Compound 20)

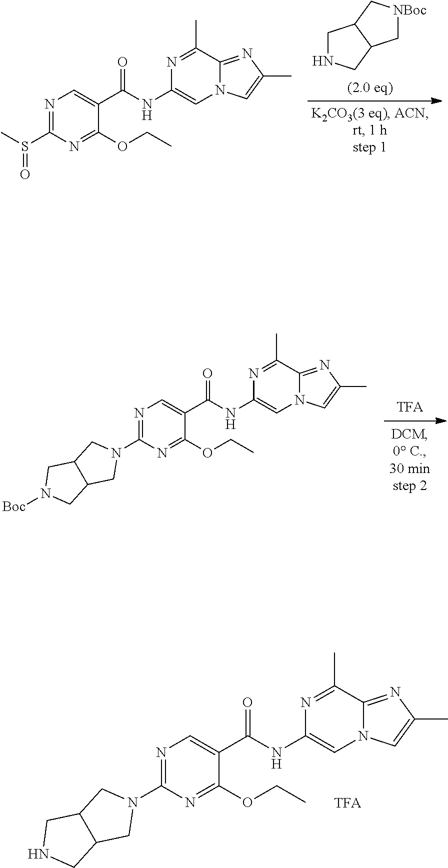

### Step 1: Preparation of ter-butyl 5-(5-(2,8-dimethylimidazo[1,2-a]pyrazin-6-yl) carbamoyl)-4-ethoxypyrimidin-2-yl) hexahydropyrrolo[3,4-c]pyrrole-2 (1H)-carboxylate

To a solution of N-(2,8-dimethylimidazo[1,2-a]pyrazin-6-yl)-4-ethoxy-2-(methylsulfinyl)pyrimidine-S-carboxamide (80 mg, 0.212 mmol) in ACN (8 mL) was added tert-butyl hexahydropyrrolo[3,4-c]pyrrole-2 (1H)-carboxylate (90 mg, 0.424 mmol) and K₂CO₃ (59 mg, 0.424 mmol). The reaction mixture was stirred for 1 h at rt. After concentration, the residue was purified by silica gel column chromatography (EA: PE=1:5) to give title product (100 mg, Y: 90%) as a yellow solid. ESI-MS (M+H)⁺: 523.3.

### Step 2: Preparation of N-(2,8-dimethylimidazo[1,2-a]pyrazin-6-yl)-4-ethoxy-2-(hexahydropyrrolo[3,4-c]pyrrol-2 (1H)-yl)pyrimidine-5-carboxamide TFA salt

To a solution of tert-butyl 5-(5-((2,8-dimethylimidazo[1,2-a]pyrazin-6-yl) carbamoyl)-4-ethoxypyrimidin-2-yl) hexahydropyrrolo[3,4-c]pyrrole-2 (1H)-carboxylate (52 mg, 0.11 mmol) in DCM (5 mL) was added TFA (1 mL) at 0° C. The reaction mixture was stirred at rt for 30 min. After concentration, the residue was purified by pre-HPLC (MeCN/0.05% TFA in water) to give title product (32 mg, Y: 60%) as a yellow solid. ESI-MS (M+H)⁺: 413.2. ¹H NMR (400 MHZ, DMSO)δ 9.86 (s, 1H), 9.24 (s, 1H), 9.00 (s, 2H), 8.76 (s, 1H), 8.07 (s, 1H), 4.60 (q, J=7.0 Hz, 2H), 3.86-3.75 (m, 2H), 3.62 (d, J=11.7 Hz, 2H), 3.45 (d, J=4.9 Hz, 2H), 3.16 (br s, 4H), 2.71 (s, 3H), 2.43 (s, 4H), 1.49 (t, J=7.0 Hz, 3H).

### Example 21. N-{8-fluoro-2-methylimidazo[1,2-a]pyridin-6-yl}-2-(piperazin-1-yl)pyrimidine-5-carboxamide (Compound 21)

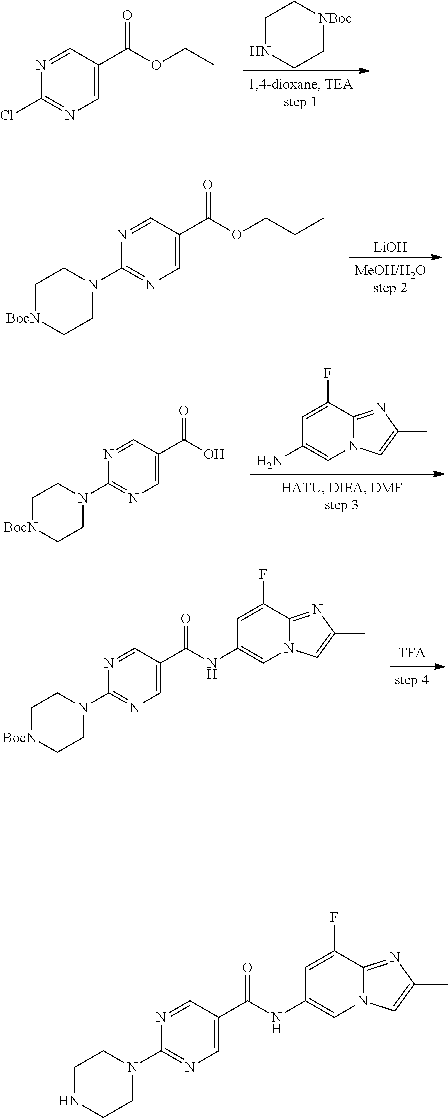

### Step 1: synthesis of ethyl 2-(4-(tert-butoxycarbonyl) piperazin-1-yl)pyrimidine-5-carboxylate

To a suspension of ethyl 2-chloropyrimidine-5-carboxylate (3.0 g, 16.3 mmol) and tert-butyl piperazine-1-carboxylate (3.3 g, 17.9 mmol) in 1,4-dixoane (30 mL) was added TEA (4.9 g, 48.9 mmol), and the reaction mixture was stirred for 16 h at 60° C. After cooling to rt, the mixture was diluted with water (100 mL), filtered, the filter cake was washed with water (50 mL), dried, the crude title product (2.5 g, Y: 46%) as white solid was used to next step directly.

### Step 2: synthesis of 2-(4-(tert-butoxycarbonyl) piperazin-1-yl)pyrimidine-5-carboxylic acid

A mixture of ethyl 2-(4-(tert-butoxycarbonyl) piperazin-1-yl)pyrimidine-5-carboxylate (500 mg, 1.5 mmol) in MeOH (100 mL) and water (10 mL) was added LiOH (600 mg, 15 mmol). The reaction mixture was stirred for 2 h at rt. After concentration, the residue was diluted with water (30 mL), adjusted to pH=3 and the precipitate was filtered. The filter cake was washed with water (20 ml) and dried to provide title product (200 mg, Y: 43%) as white solid.

¹H NMR (400 MHZ, DMSO)δ 8.82 (s, 2H), 3.89-3.86 (m, 4H), 3.47 (d, J==5.5 Hz, 4H), 1.47 (s, 9H).

### Step 3: synthesis of tert-butyl 4-(5-((8-fluoro-2-methylimidazo[1,2-a]pyridin-6-yl) carbamoyl)pyrimidin-2-yl) piperazine-1-carboxylate

To a solution of 2-(4-(tert-butoxycarbonyl) piperazin-1-yl)pyrimidine-5-carboxylic acid (200 mg, 0.65 mmol) in DMF (8 mL) was added HATU (250 mg, 0.8 mmol), DIEA (160 mg, 1.3 mmol), the reaction mixture was stirred 30 min at rt. Then 8-fluoro-2-methylimidazo[1,2-a]pyridin-6-amine (165 mg, 1.0 mmol) was added and the reaction was stirred for another 2 hours. The mixture was diluted with water (50 mL) and the solid was filtered. The solid was washed with water and dried to provide title product (100 mg, Y: 23%) as brown solid. ESI-MS: [M+H]⁺456.5

### Step 4: synthesis of N-(8-fluoro-2-methylimidazo[1,2-a]pyridin-6-yl)-2-(piperazin-1-yl)pyrimidine-5-carboxamide

To a mixture of tert-butyl 4-(5-((8-fluoro-2-methylimidazo[1,2-a]pyridin-6-yl) carbamoyl)pyrimidin-2-yl) piperazine-1-carboxylate (100 mg, 0.22 mmol) in DCM (3 mL) was added TFA (1.0 mL), the reaction mixture was stirred for 1 h at rt. Concentrated, the residue was purified by pre-HPLC (MeCN/0.05% NH₃.H₂O in water) to give title product as white solid (15 mg, yield: 9%). ESI-MS: [M+H]⁺356.0 ¹H NMR (400 MHZ, DMSO)δ 10.14 (s, 1H), 9.03 (d, J=1.5 Hz, 1H), 8.88 (s, 2H), 7.90 (d, J=2.7 Hz, 1H), 7.26 (dd, J=12.6, 1.6 Hz, 1H), 3.80-3.76 (m, 4H), 2.77-2.73 (m, 4H), 2.34 (s, 3H).

### Example 22. N-{2,8-dimethylimidazo[1,2-a]pyrazin-6-yl}-4-methoxy-2-{octahydropyrrolo[3,4-c]pyrrol-2-yl}pyrimidine-5-carboxamide (Compound 22)

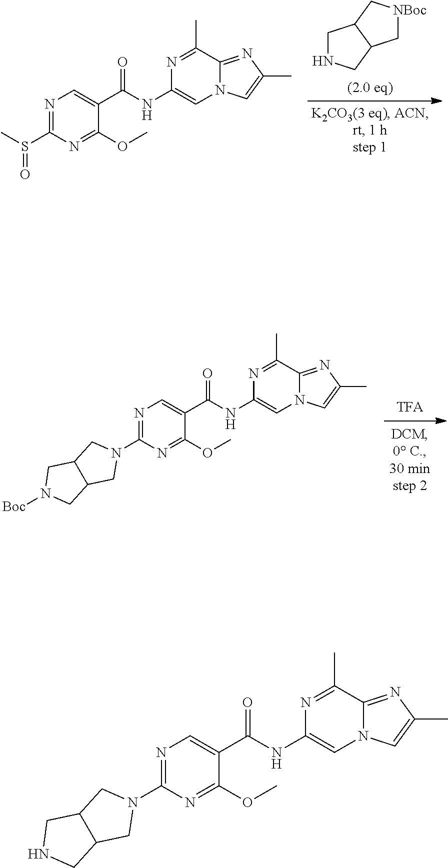

### Step 1: Preparation of tert-butyl 5-(5-((2,8-dimethylimidazo[1,2-a]pyrazin-6-yl) carbamoyl)-4-methoxypyrimidin-2-yl) hexahydropyrrolo[3,4-c]pyrrole-2 (1H)-carboxylate

To a solution of N-(2,8-dimethylimidazo[1,2-a]pyrazin-6-yl)-4-ethoxy-2-(methylsulfinyl)pyrimidine-5-carboxamide (76 mg, 0.212 mmol) in ACN (8 mL) was added tert-butyl hexahydropyrrolo[3,4-c]pyrrole-2 (1H)-carboxylate (90 mg, 0.424 mmol) and K₂CO₃ (59 mg, 0.424 mmol). The reaction mixture was stirred for 1 h at rt. After concentration, the residue was purified by silica gel column chromatography (EA: PE=1:5) to give title product (51 mg, Y: 47%) as a yellow solid. ESI-MS (M+H)⁺: 509.2.

### Step 2: Preparation of N-(2,8-dimethylimidazo[1,2-a]pyrazin-6-yl)-2-(hexahydropyrrolo[3,4-c]pyrrol-2 (1H)-yl)-4-methoxypyrimidine-5-carboxamide TFA salt

To a solution of tert-butyl 5-(5-((2,8-dimethylimidazo[1,2-a]pyrazin-6-yl) carbamoyl)-4-methoxypyrimidin-2-yl) hexahydropyrrolo[3,4-c]pyrrole-2 (1H)-carboxylate (51 mg, 0.1 mmol) in DCM (5 mL) was added TFA (1 mL) at 0° C. The reaction mixture was stirred at rt for 30 min. After concentration, the residue was purified by pre-HPLC (MeCN/0.05% TFA in water) to give title product (30 mg, Y: 57%) as a yellow solid. ESI-MS (M+H)⁺: 409.2. ¹H NMR (400 MHZ, MeOD-da) δ 9.09 (s, 1H), 8.78 (s, 1H), 7.75 (s, 1H), 4.19 (s, 3H), 3.86 (d, J=9.0 Hz, 2H), 3.56 (d, J=10.6 Hz, 2H), 3.14 (dd, J=11.2, 7.1 Hz, 2H), 3.00 (br s, 2H), 2.82-2.75 (m, 2H), 2.74 (s, 3H), 2.44 (s, 3H) . . .

### Example 25. 4-ethoxy-N-(2-methyl-2H-indazol-5-yl)-2-(piperazin-1-yl)pyrimidine-5-carboxamide (Compound 25)

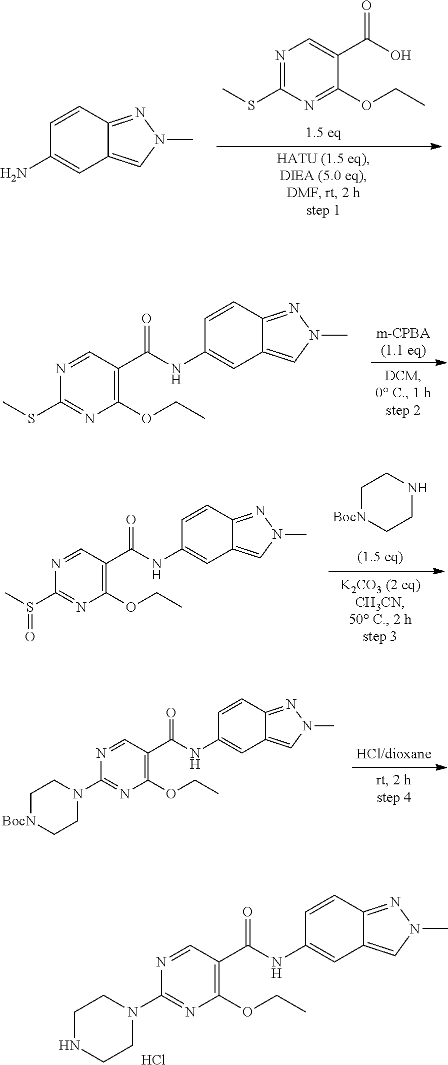

### Step 1: preparation of 4-ethoxy-N-(2-methyl-2H-indazol-5-yl)-2-(methylthio)pyrimidine-5-carboxamide

A mixture of 2-methyl-2H-indazol-5-amine (100 mg, 0.680 mmol), HATU (388 mg, 1.02 mmol), 4-ethoxy-2-(methylthio)pyrimidine-5-carboxylic acid (175 mg, 0.816 mmol) and DIEA (175 mg, 1.36 mmol) in DMF (5 mL) was stirred at r.t. for 2 h. The reaction mixture was diluted with water (30 mL), stirred at r.t. for 30 min, the precipitate was filtered and concentrated in vacuo to give 4-ethoxy-N-(2-methyl-2H-indazol-5-yl)-2-(methylthio)pyrimidine-5-carboxamide (200 mg, 85.8% yield) as a yellow solid. ¹H NMR (400 MHz, DMSO-d₆) δ 9.98 (s, 1H), 8.70 (s, 1H), 8.30 (s, 1H), 8.25 (s, 1H), 7.59 (d, J=9.2 Hz, 1H), 7.30 (d, J=9.2 Hz, 1H), 4.55 (q, J=7.0 Hz, 2H), 4.15 (s, 3H), 2.58 (s, 3H), 1.42 (t, J=7.0 Hz, 3H). ESI-MS (M+H)⁺: 344.1

### Step 2: preparation of 4-ethoxy-N-(2-methyl-2H-indazol-5-yl)-2-(methylsulfinyl)pyrimidine-5-carboxamide

To a solution of 4-ethoxy-N-(2-methyl-2H-indazol-5-yl)-2-(methylthio)pyrimidine-5-carboxamide (100 mg, 0.292 mmol) in DCM (5 ml) was added m-CPBA (78.8 mg, 0.321 mmol) at 0° C. and the mixture was stirred at this temperature for 1 h. To the mixture was added sat. NaHSO₄ (10 mL) and the mixture was extracted with DCM (20 mL*2). The combined organic layer was washed with brine (50 mL), filtered and concentrated in vacuo to give 4-ethoxy-N-(2-methyl-2H-indazol-5-yl)-2-(methylsulfinyl)pyrimidine-5-carboxamide (120 mg, crude) as a yellow solid, which was used in next step directly. ESI-MS: [M+H]⁺360.1

### Step 3: Preparation of tert-butyl 4-(4-ethoxy-5-((2-methyl-2H-indazol-5-yl) carbamoyl)pyrimidin-2-yl) piperazine-1-carboxylate

To a solution of compound 4-ethoxy-N-(2-methyl-2H-indazol-5-yl)-2-(methylsulfinyl)pyrimidine-5-carboxamide (120 mg. 0.334 mmol) and tert-butyl piperazine-1-carboxylate (93.2 mg, 0.501 mmol) in CH₃CN (5 mL) was added K₂CO₃ (92.2 mg, 0.668 mmol), the reaction mixture was stirred at 50° C. for 2 hours. The mixture was diluted with EA (80 mL), washed with water (20 mL) and brine (20 mL), dried over sodium sulfate, filtered and concentrated in vacuo. The residue was purified by column chromatography (PE/EA=1:1) to give tert-butyl 4-(4-ethoxy-5-((2-methyl-2H-indazol-5-yl) carbamoyl)pyrimidin-2-yl) piperazine-1-carboxylate (100 mg, yield: 62.5%) as a yellow solid. ¹H NMR (400 MHz, CDCl₃)δ 9.41 (s, 1H), 9.05 (s, 1H), 8.42 (s, 1H), 7.85 (s, 1H), 7.65 (d, J=9.1 Hz, 1H), 7.05 (d, J=9.1 Hz, 1H), 4.60 (q, J=7.0 Hz, 2H), 4.21 (s, 3H), 3.96-3.81 (m, 4H), 3.56-3.46 (m, 4H), 1.60 (t. J=7.0 Hz, 3H), 1.50 (s, 9H). ESI-MS (M+H)⁺: 482.3.

### Step 4: N-(2,8-dimethylimidazo[1,2-a]pyrazin-6-yl)-2-(hexahydropyrrolo[3,4-c]pyrrol-2 (1H)-yl)-4-hydroxypyrimidine-5-carboxamide hydrochloride

To a solution of tert-butyl 4-(4-ethoxy-5-((2-methyl-2H-indazol-5-yl) carbamoyl)pyrimidin-2-yl) piperazine-1-carboxylate (100 mg, 0.208 mmol) in 1,4-dioxane (2 ml) was added 4M HCl/dioxane (2 ml), the mixture was stirred at room temperature for 2 hours. The precipitate was filtered and concentrated in vacuo to give 4-ethoxy-N-(2-methyl-2H-indazol-5-yl)-2-(piperazin-1-yl)pyrimidine-5-carboxamidehydrochloride (67 mg, yield: 71.3%) as a yellow solid. ¹H NMR (400 MHZ, DMSO-d₆) δ 9.71-9.69 (m, 3H), 8.66 (s, 1H), 8.33 (s, 1H), 8.24 (s, 1H), 7.58 (d, J=9.1 Hz, 1H), 7.33 (d, J=9.1 Hz, 1H), 4.54 (q, J=7.0 Hz, 2H), 4.15 (s, 3H), 4.08 (br.s, 4H), 3.18 (br.s, 4H), 1.44 (t, J=7.0 Hz, 3H). ESI-MS: [M+H]⁺382.2.

### Example 26. 4-ethoxy-N-(7-fluoro-2-methyl-2H-indazol-5-yl)-2-(piperazin-1-yl)pyrimidine-5-carboxamide (Compound 26)

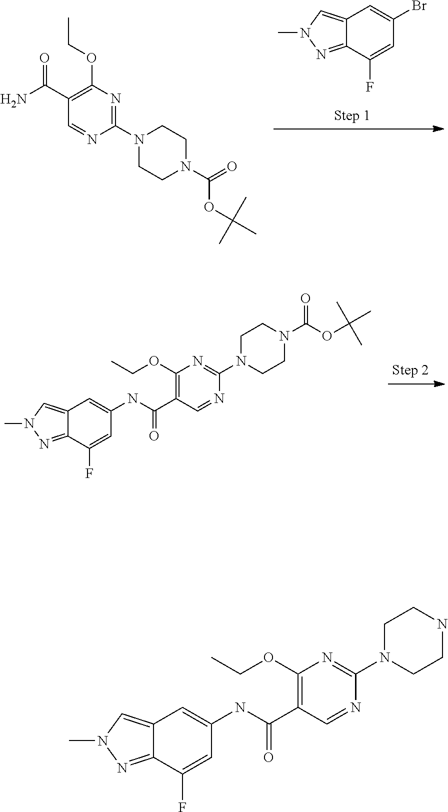

### Step 1: Preparation of tert-butyl 4-4-ethoxy-5-[(7-fluoro-2-methyl-2H-indazol-5-yl) carbamoyl]pyrimidin-2-ylpiperazine-1-carboxylate

tert-Butyl 4-(5-carbamoyl-4-ethoxypyrimidin-2-yl) piperazine-1-carboxylate (199.78 mg, 568.53 μmol), 5-bromo-7-fluoro-2-methyl-2H-indazole (130.22 mg, 568.53 μmol), tris((1E,4E)-1,5-diphenylpenta-1,4-dien-3-one) dipalladium (52.06 mg, 56.85 μmol), [5-(diphenylphosphanyl)-9,9-dimethyl-9H-xanthen-4-yl]diphenyl-phosphane (65.79 mg, 113.71 μmol), and cesium carbonate (555.71 mg, 1.71 mmol) were mixed in dioxane under an argon atmosphere. The reaction mixture was heated to 100° C. for 17 h. The resulting mixture was evaporated under reduced pressure and purified by HPLC to obtain tert-butyl 4-4-ethoxy-5-[(7-fluoro-2-methyl-2H-indazol-5-yl) carbamoyl]pyrimidin-2-ylpiperazine-1-carboxylate (120.0 mg, Y.: 42.3%). ESI-MS (M+H)⁺: 500.4.

### Step 2: Preparation of 4-ethoxy-N-(7-fluoro-2-methyl-2H-indazol-5-yl)-2-(piperazin-1-yl)pyrimidine-5-carboxamide

tert-Butyl 4-4-ethoxy-5-[(7-fluoro-2-methyl-2H-indazol-5-yl) carbamoyl]pyrimidin-2-ylpiperazine-1-carboxylate (119.0 mg, 238.22 μmol) was dissolved in DCM (1 mL) and TFA (1 mL) and the reaction mixture was stirred overnight at r.t. The resulting mixture was evaporated under reduced pressure and purified by HPLC to obtain pure 4-ethoxy-N-(7-fluoro-2-methyl-2H-indazol-5-yl)-2-(piperazin-1-yl)pyrimidine-5-carboxamide as TFA salt (79.1 mg, Y.: 64.8%). ESI-MS (M+H)⁺: 400.2. ¹H NMR (400 MHZ, DMSO-d₆) δ 9.62 (s, 1H), 8.78 (br s, 2H), 8.64 (s, 1H), 8.63 (s, 1H), 8.38 (s, 1H), 7.94 (s, 1H), 7.25 (d, J=8 Hz, 1H), 4.51 (q, J=7.2 Hz, 2H), 4.14 (s, 3H), 4.01-3.95 (m, 4H), 3.20-3.15 (m, 4H), 1.39 (t, J=7.1 Hz, 3H).

### Example 27. 4-ethoxy-N-{8-methoxy-2-methylimidazo[1,2-a]pyridin-6-yl}-2-(piperazin-1-yl)pyrimidine-5-carboxamide (Compound 27)

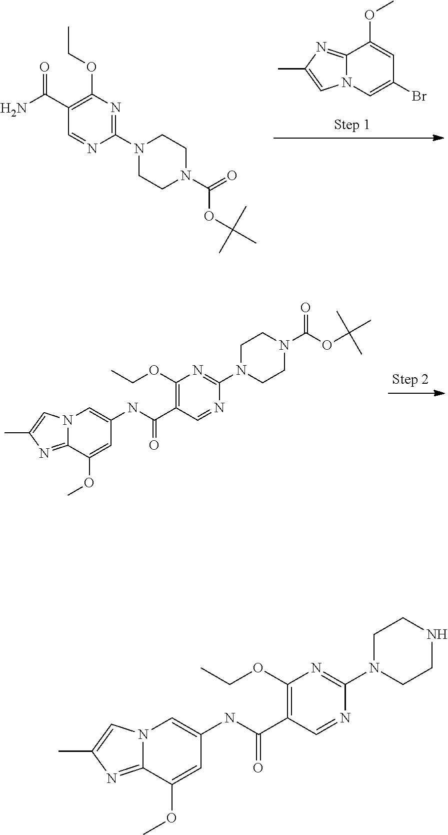

### Step 1: Preparation of tert-butyl 4-[4-ethoxy-5-(8-methoxy-2-methylimidazo[1,2-a]pyridin-6-ylcarbamoyl)pyrimidin-2-yl]piperazine-1-carboxylate

tert-Butyl 4-(5-carbamoyl-4-ethoxypyrimidin-2-yl) piperazine-1-carboxylate (199.89 mg, 568.83 μmol), 6-bromo-8-methoxy-2-methylimidazo[1,2-a]pyridine (137.14 mg, 568.83 μmol), tris((1E,4E)-1,5-diphenyl-penta-1,4-dien-3-one) dipalladium (52.09 mg, 56.88 μmol), [5-(diphenylphosphanyl)-9,9-dimethyl-9H-xanthen-4-yl]diphenylphosphane (65.83 mg, 113.77 μmol), and cesium carbonate (556.01 mg, 1.71 mmol) were mixed in dioxane under an argon atmosphere. The reaction mixture was heated to 100° C. for 17 h. The resulting mixture was evaporated under reduced pressure and purified by HPLC to obtain tert-butyl 4-[4-ethoxy-5-(8-methoxy-2-methylimidazo[1,2-a]pyridin-6-ylcarbamoyl)pyrimidin-2-yl]piperazine-1-carboxylate (200.0 mg, Y.: 68.7%). ESI-MS (M+H)⁺: 512.2.

### Step 2: Preparation of 4-ethoxy-N-8-methoxy-2-methylimidazo[1,2-a]pyridin-6-yl-2-(piperazin-1-yl)pyrimidine-5-carboxamide; trifluoroacetic acid

tert-Butyl 4-[4-ethoxy-5-(8-methoxy-2-methylimidazo[1,2-a]pyridin-6-ylcarbamoyl)pyrimidin-2-yl]piperazine-1-carboxylate (99.3 mg, 194.11 μmol) was dissolved in DCM (1 mL) and TFA (1 mL) and the reaction mixture was stirred overnight at r.t. The resulting mixture was evaporated under reduced pressure and purified by HPLC to obtain pure 4-ethoxy-N-8-methoxy-2-methylimidazo[1,2-a]pyridin-6-yl-2-(piperazin-1-yl)pyrimidine-5-carboxamide as TFA salt (84.4 mg, Y.: 82.7%). ESI-MS (M+H)⁺: 412.2. ¹H NMR (400 MHZ, DMSO-d₆)10.01 (s, 1H), 9.19 (s, 1H), 8.99 (br s, 2H), 8.63 (s, 1H), 8.14 (s, 1H), 7.41 (s, 1H), 4.51 (q, J=7.2 Hz, 2H), 4.04 (s, 3H)4.02-3.97 (m, 4H), 3.22-3.14 (m, 4H), 2.40 (s, 3H), 1.39 (t, J=7.1 Hz, 3H).

### Example 28. 4-ethoxy-N-{2-methylimidazo[1,2-a]pyrazin-6-yl}-2-{octahydropyrrolo[3,4-c]pyrrol-2-yl}pyrimidine-5-carboxamide (Compound 28)

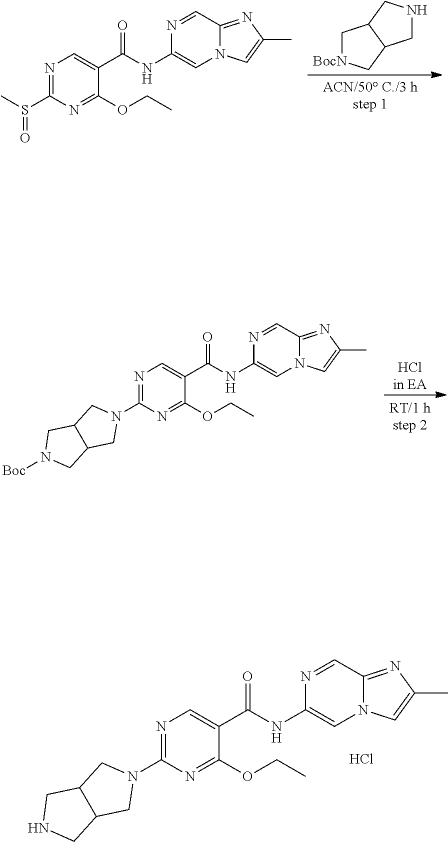

### Step 1: tert-butyl 5-(4-ethoxy-5-((2-methylimidazo[1,2-a]pyrazin-6-yl) carbamoyl)pyrimidin-2-yl) hexahydropyrrolo[3,4-c]pyrrole-2 (1H)-carboxylate

To a solution of 4-ethoxy-N-(2-methylimidazo[1,2-a]pyrazin-6-yl)-2-(methylsulfinyl)pyrimidine-5-carboxamide (100 mg, 0.278 mmol) in ACN (5 mL) was added tert-butyl hexahydropyrrolo[3,4-c]pyrrole-2 (1H)-carboxylate (117 mg, 0.56 mmol) and K₂CO₃ (115 mg, 0.833 mmol). The reaction mixture was stirred for 3 h at 50° C. After concentration, the residue was purified by prep-HPLC (0.05% HCl in water/CH₃CN) to give the production (60 mg, Y: 42.5%) as a yellow solid. ESI-MS (M+H)⁺: 509.3. ¹H NMR (400 MHz, CDCl₃) δ 9.83 (s, 1H), 9.27 (d, J=1.3 Hz, 1H), 9.01 (s, 1H), 8.74 (s, 1H), 7.47 (s, 1H), 4.66 (q, J=7.1 Hz, 2H), 3.90 (br.s, 2H), 3.76-3.52 (m, 4H), 3.44-3.22 (m, 2H), 3.01 (br.s, 2H), 2.51 (s, 3H), 1.59 (t, J=7.1 Hz, 3H), 1.46 (s, 9H).

### Step 2:4-ethoxy-2-(hexahydropyrrolo[3,4-c]pyrrol-2 (1H)-yl)-N-(2-methylimidazo[1,2-a]pyrazin-6-yl)pyrimidine-5-carboxamide hydrogen chloride

To a solution of tert-butyl 5-(4-ethoxy-5-((2-methylimidazo[1,2-a]pyrazin-6-yl) carbamoyl)pyrimidin-2-yl) hexahydro pyrrolo[3,4-c]pyrrole-2 (1H)-carboxylate (60 mg, 0.118 mmol) in EA (5 mL) was added HCl in EA (5 mL) at RT. The reaction mixture was stirred at rt for 1 h. The resulting mixture was concentrated and the crude was purified by pre-HPLC to give title product as hydrogen chloride (6 mg, Y: 12.4%) as a yellow solid. ESI-MS (M+H)⁺: 409.1.1H NMR (400 MHz, MeOD-d₄) δ 9.29-9.25 (m, 1H), 8.84-8.80 (m, 1H), 8.68 (d, J=1.5 Hz, 1H), 7.84 (s, 1H), 4.69-4.66 (m, 2H), 3.86 (br.s, 2H), 3.57 (d, J=12.3 Hz, 2H), 3.19-3.12 (m, 2H), 3.01 (br.s, 2H), 2.80 (d, J=11.0 Hz, 2H), 2.46 (s, 3H), 1.56 (t, J=7.1 Hz, 3H).

### Example 29. Parallel synthesis of Compounds 32-106

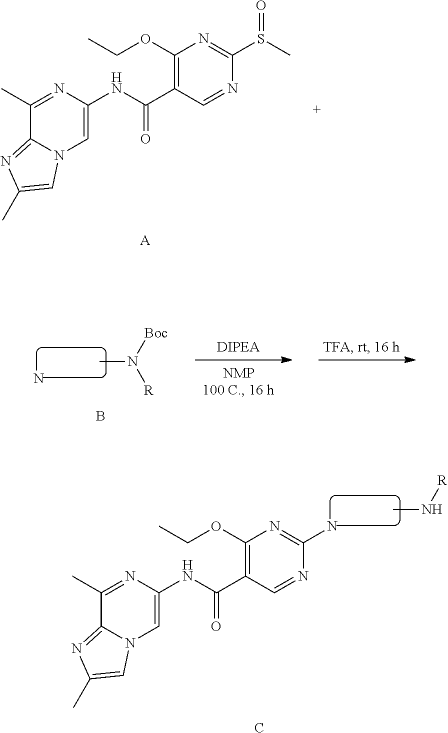

To a mixture of 1 eq. of ArSOMe (A) and 2 eq. amine (B) in Iml NMP was added 5.0 eq. * DIPEA. The reaction mixture was stirred at 100° C. for 16 hours. After cooling to room temperature, the solvent was evaporated and 1 ml of cleavage cocktail ** was added. The mixture was left to react for 4 h and evaporated to dryness. The residue was dissolved in DMSO and subjected to HPLC purification ***

*In case of using Reagent 1 and/or Reagent 2 as salt, an additional amount DIPEA (1.1 eq. for each eq. of acid) was added to the reaction mixture to convert the reagent into a free form.

** The cleavage cocktail was prepared by mixing TFA (92.5% v/v), water (5% v/v) and TIPS (2.5% v/v).

*** Resulting solution was purified by HPLC (Deionized Water (phase A) and HPLC-grade Acetonitrile (phase B) were used as an eluent to obtained final compound (D). In most cases, TFA was used as an additive.

Instrument: Agilent 1260 Infinity systems equipped with DAD and mass-detector

Column: Waters Sunfire CIS OBD Prep Column, 100 A, 5 μm, 19 mm×100 mm with SunFire C18 Prep Guard Cartridge, 100 A, 10 μm, 19 mm×10 mm.

| Compound | | ESI-MS |
|---|---|---|
| No. | Structure | (M + H)⁺ |
| 32 | 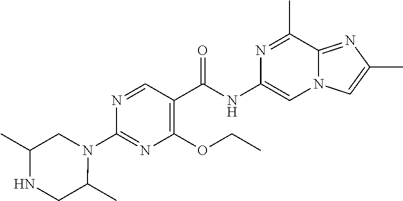 | 425.1 |
| 33 | 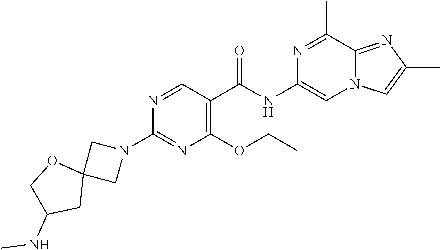 | 453.4 |
| 34 | 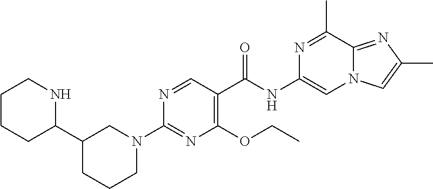 [SMILES: CCOc1nc(N2CCCC(C3CCCCN3)C2)ncc1C(=O)Nc1cn2cc(C)nc2c(C)n1] | 479.4 |
| 35 | 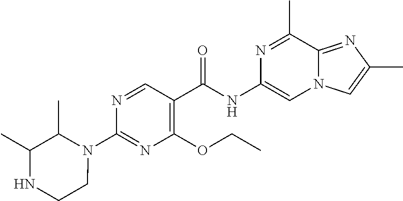 | 425.3 |
| 36 | 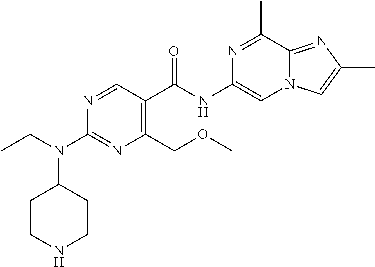 [SMILES: CCN(c1ncc(C(=O)Nc2cn3cc(C)nc3c(C)n2)c(COC)n1)C1CCNCC1] | 439.4 |
| 37 | 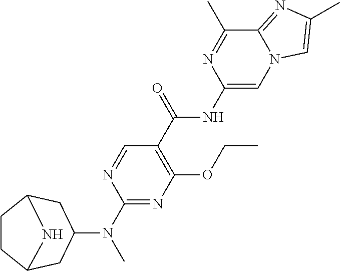 [SMILES: CCOc1nc(N(C)C2CC3CCC(C2)N3)ncc1C(=O)Nc1cn2cc(C)nc2c(C)n1] | 451.2 |
| 38 | 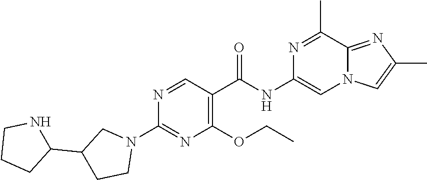 [SMILES: CCOc1nc(N2CCC(C3CCCN3)C2)ncc1C(=O)Nc1cn2cc(C)nc2c(C)n1] | 451.2 |
| 39 | 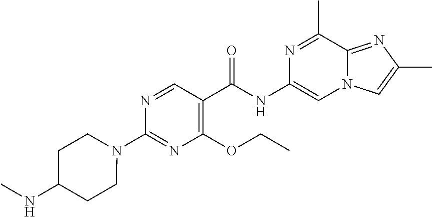 | 425.3 |
| 40 | 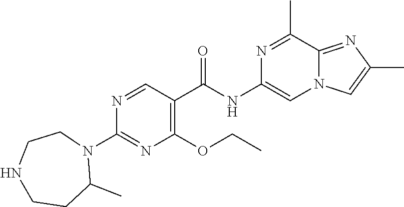 [SMILES: CCOc1nc(N2CCNCCC2C)ncc1C(=O)Nc1cn2cc(C)nc2c(C)n1] | 425.3 |
| 41 | 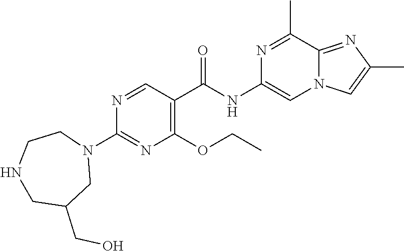 [SMILES: CCOc1nc(N2CCNCC(CO)C2)ncc1C(=O)Nc1cn2cc(C)nc2c(C)n1] | 443.0 |
| 42 | 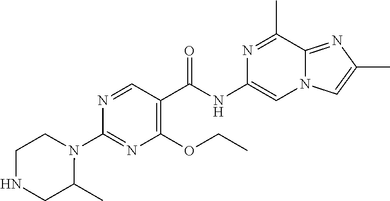 [SMILES: CCOc1nc(N2CCNCC2C)ncc1C(=O)Nc1cn2cc(C)nc2c(C)n1] | 411.4 |
| 43 | 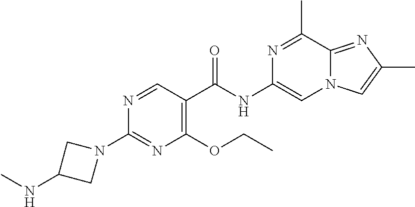 | 397.2 |
| 44 | 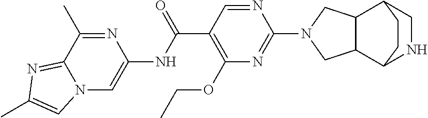 [SMILES: CCOc1nc(N2CC3C4CCC(NC4)C3C2)ncc1C(=O)Nc1cn2cc(C)nc2c(C)n1] | 463.4 |
| 45 | 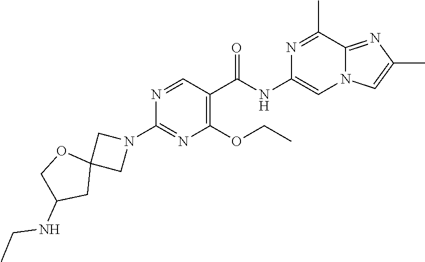 [SMILES: CCNC1COC2(C1)CN(c1ncc(C(=O)Nc3cn4cc(C)nc4c(C)n3)c(OCC)n1)C2] | 467.3 |
| 46 | 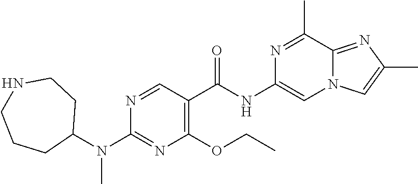 [SMILES: CCOc1nc(N(C)C2CCCNCC2)ncc1C(=O)Nc1cn2cc(C)nc2c(C)n1] | 439.3 |
| 47 | 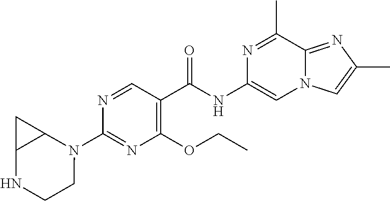 [SMILES: CCOc1nc(N2CCNC3CC32)ncc1C(=O)Nc1cn2cc(C)nc2c(C)n1] | 409.0 |
| 48 | 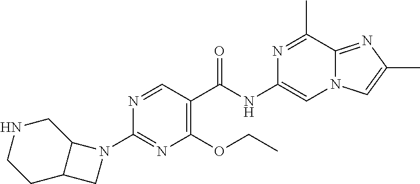 [SMILES: CCOc1nc(N2CC3CCNCC32)ncc1C(=O)Nc1cn2cc(C)nc2c(C)n1] | 423.2 |
| 49 | 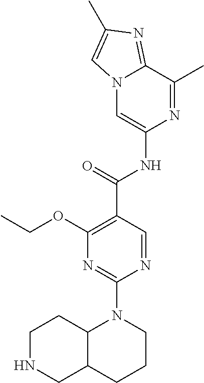 [SMILES: CCOc1nc(N2CCCC3CNCCC32)ncc1C(=O)Nc1cn2cc(C)nc2c(C)n1] | 451.2 |
| 50 | 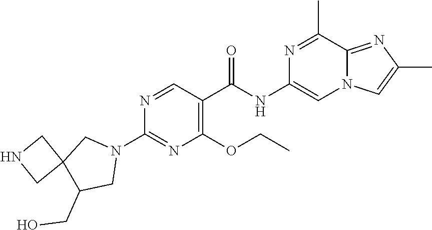 [SMILES: CCOc1nc(N2CC(CO)C3(CNC3)C2)ncc1C(=O)Nc1cn2cc(C)nc2c(C)n1] | 453.5 |
| 51 | 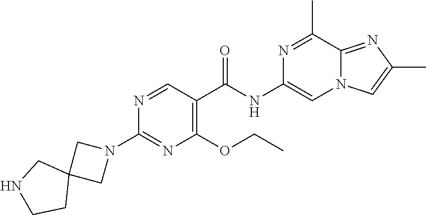 [SMILES: CCOc1nc(N2CC3(CCNC3)C2)ncc1C(=O)Nc1cn2cc(C)nc2c(C)n1] | 423.2 |
| 52 | 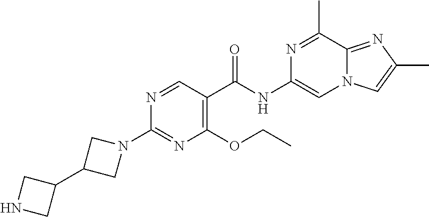 [SMILES: CCOc1nc(N2CC(C3CNC3)C2)ncc1C(=O)Nc1cn2cc(C)nc2c(C)n1] | 423.2 |
| 53 | 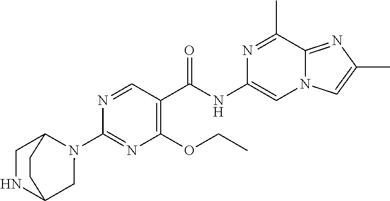 [SMILES: CCOc1nc(N2CC3CCC2CN3)ncc1C(=O)Nc1cn2cc(C)nc2c(C)n1] | 423.2 |
| 54 | 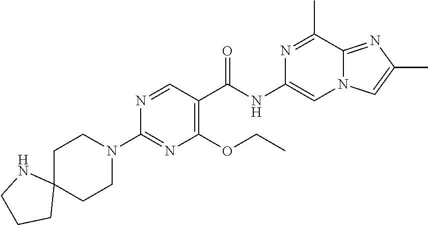 [SMILES: CCOc1nc(N2CCC3(CCCN3)CC2)ncc1C(=O)Nc1cn2cc(C)nc2c(C)n1] | 451.3 |
| 55 | 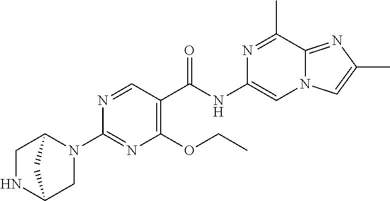 [SMILES: CCOc1nc(N2C[C@@H]3C[C@H]2CN3)ncc1C(=O)Nc1cn2cc(C)nc2c(C)n1] | 408.3 |
| 56 | 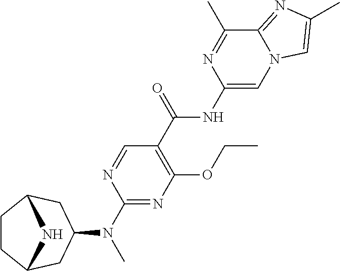 [SMILES: CCOc1nc(N(C)[C@@H]2C[C@H]3CC[C@@H](C2)N3)ncc1C(=O)Nc1cn2cc(C)nc2c(C)n1] | 451.3 |
| 57 | 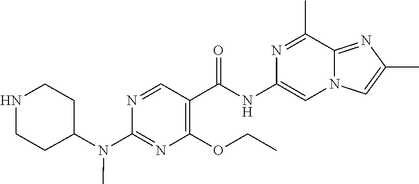 [SMILES: CCOc1nc(N(C)C2CCNCC2)ncc1C(=O)Nc1cn2cc(C)nc2c(C)n1] | 425.3 |
| 58 | 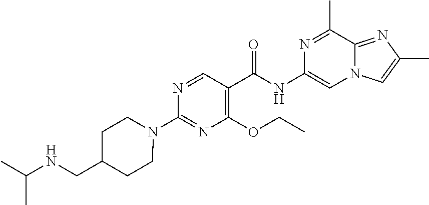 [SMILES: CCOc1nc(N2CCC(CNC(C)C)CC2)ncc1C(=O)Nc1cn2cc(C)nc2c(C)n1] | 467.4 |
| 59 | 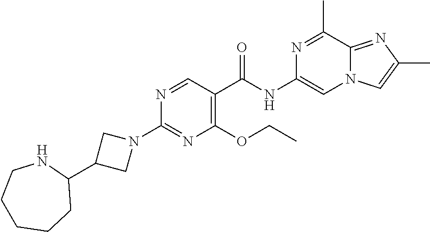 [SMILES: CCOc1nc(N2CC(C3CCCCCN3)C2)ncc1C(=O)Nc1cn2cc(C)nc2c(C)n1] | 465.5 |
| 60 | 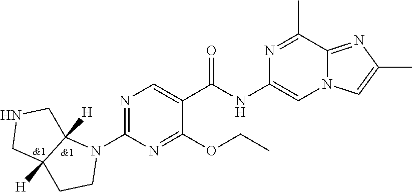 [SMILES: CCOc1nc(N2CC[C@@H]3CNC[C@@H]32)ncc1C(=O)Nc1cn2cc(C)nc2c(C)n1] | 423.2 |
| 61 | 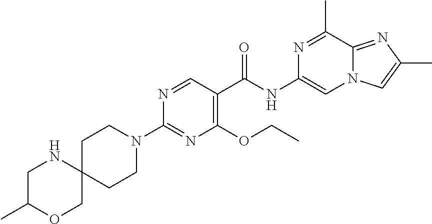 | 481.4 |
| 62 | 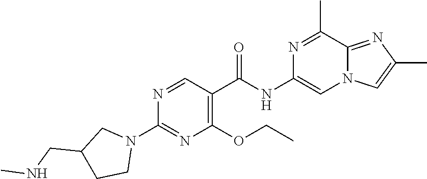 | 423.2 |
| 63 | 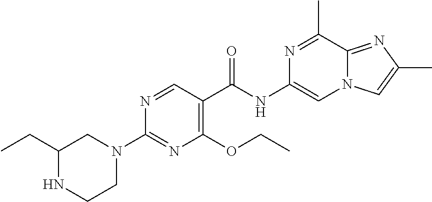 [SMILES: CCOc1nc(N2CCNC(CC)C2)ncc1C(=O)Nc1cn2cc(C)nc2c(C)n1] | 425.4 |
| 64 | 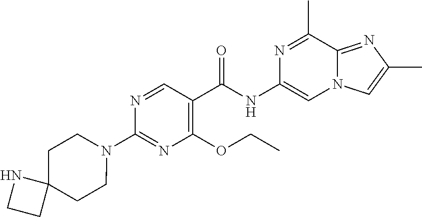 [SMILES: CCOc1nc(N2CCC3(CCN3)CC2)ncc1C(=O)Nc1cn2cc(C)nc2c(C)n1] | 436.4 |
| 65 | 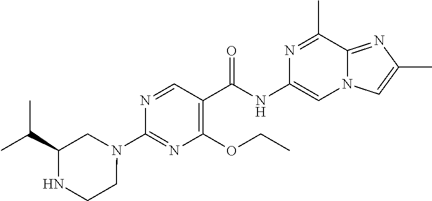 [SMILES: CCOc1nc(N2CCN[C@@H](C(C)C)C2)ncc1C(=O)Nc1cn2cc(C)nc2c(C)n1] | 439.5 |
| 66 | 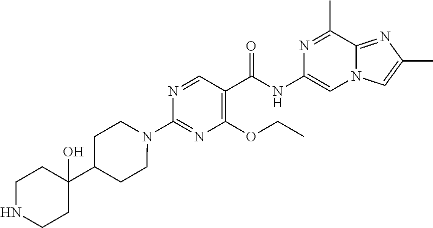 [SMILES: CCOc1nc(N2CCC(C3(O)CCNCC3)CC2)ncc1C(=O)Nc1cn2cc(C)nc2c(C)n1] | 495.5 |
| 67 | 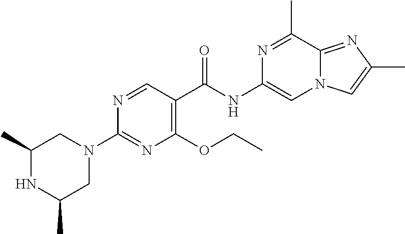 | 425.5 |
| 68 | 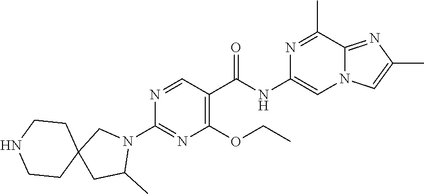 [SMILES: CCOc1nc(N2CC3(CCNCC3)CC2C)ncc1C(=O)Nc1cn2cc(C)nc2c(C)n1] | 465.4 |
| 69 | 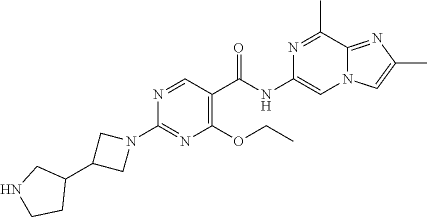 [SMILES: CCOc1nc(N2CC(C3CCNC3)C2)ncc1C(=O)Nc1cn2cc(C)nc2c(C)n1] | 437.1 |
| 70 | 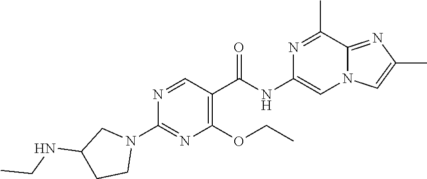 [SMILES: CCNC1CCN(c2ncc(C(=O)Nc3cn4cc(C)nc4c(C)n3)c(OCC)n2)C1] | 425.4 |
| 71 | 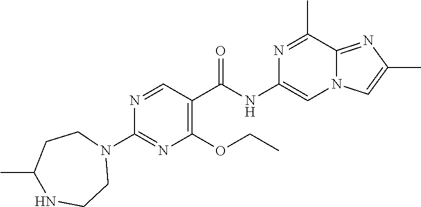 | 424.2 |
| 72 | 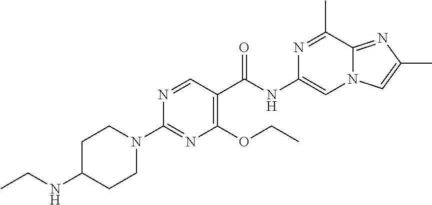 [SMILES: CCNC1CCN(c2ncc(C(=O)Nc3cn4cc(C)nc4c(C)n3)c(OCC)n2)CC1] | 438.3 |
| 73 | 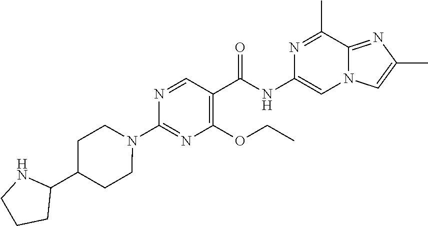 [SMILES: CCOc1nc(N2CCC(C3CCCN3)CC2)ncc1C(=O)Nc1cn2cc(C)nc2c(C)n1] | 464.4 |
| 74 | 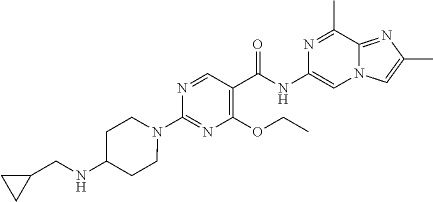 [SMILES: CCOc1nc(N2CCC(NCC3CC3)CC2)ncc1C(=O)Nc1cn2cc(C)nc2c(C)n1] | 465.4 |
| 75 | 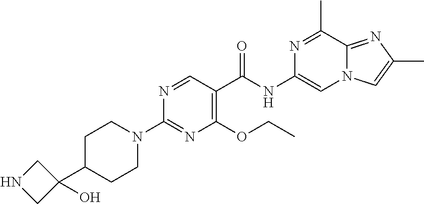 [SMILES: CCOc1nc(N2CCC(C3(O)CNC3)CC2)ncc1C(=O)Nc1cn2cc(C)nc2c(C)n1] | 467.4 |
| 76 | 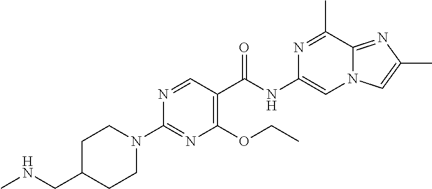 | 439.3 |
| 77 | 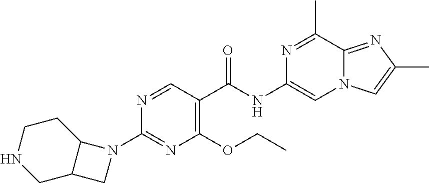 [SMILES: CCOc1nc(N2CC3CNCCC32)ncc1C(=O)Nc1cn2cc(C)nc2c(C)n1] | 423.4 |
| 78 | 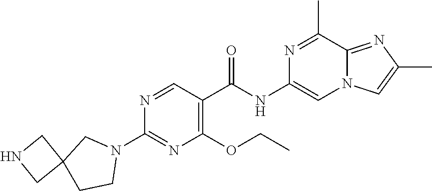 [SMILES: CCOc1nc(N2CCC3(CNC3)C2)ncc1C(=O)Nc1cn2cc(C)nc2c(C)n1] | 423.3 |
| 79 | 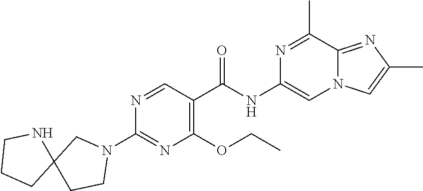 [SMILES: CCOc1nc(N2CCC3(CCCN3)C2)ncc1C(=O)Nc1cn2cc(C)nc2c(C)n1] | 436.2 |
| 80 | 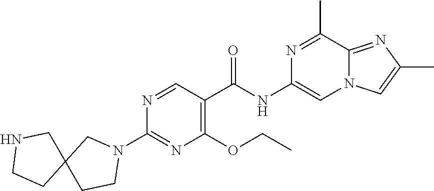 [SMILES: CCOc1nc(N2CCC3(CCNC3)C2)ncc1C(=O)Nc1cn2cc(C)nc2c(C)n1] | 437.2 |
| 81 | 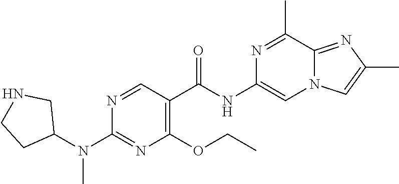 [SMILES: CCOc1nc(N(C)C2CCNC2)ncc1C(=O)Nc1cn2cc(C)nc2c(C)n1] | 411.2 |
| 82 | 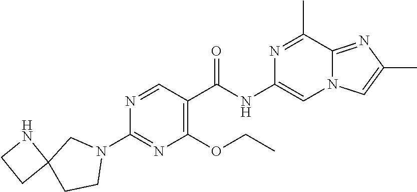 [SMILES: CCOc1nc(N2CCC3(CCN3)C2)ncc1C(=O)Nc1cn2cc(C)nc2c(C)n1] | 423.2 |
| 83 | 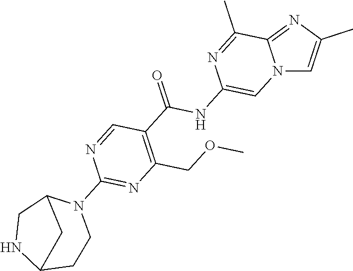 [SMILES: COCc1nc(N2CCC3CC2CN3)ncc1C(=O)Nc1cn2cc(C)nc2c(C)n1] | 423.0 |
| 84 | 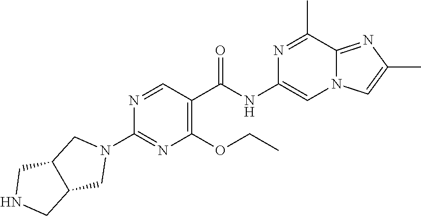 [SMILES: CCOc1nc(N2C[C@H]3CNC[C@H]3C2)ncc1C(=O)Nc1cn2cc(C)nc2c(C)n1] | 423.4 |
| 85 | 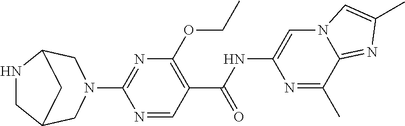 [SMILES: CCOc1nc(N2CC3CNC(C3)C2)ncc1C(=O)Nc1cn2cc(C)nc2c(C)n1] | 423.4 |
| 86 | 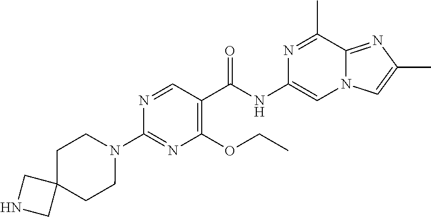 [SMILES: CCOc1nc(N2CCC3(CC2)CNC3)ncc1C(=O)Nc1cn2cc(C)nc2c(C)n1] | 437.3 |
| 87 | 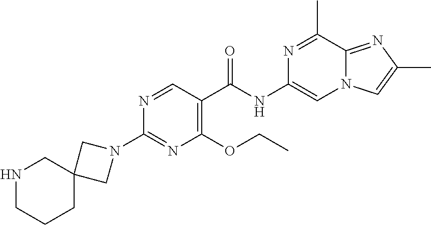 [SMILES: CCOc1nc(N2CC3(CCCNC3)C2)ncc1C(=O)Nc1cn2cc(C)nc2c(C)n1] | 437.4 |
| 88 | 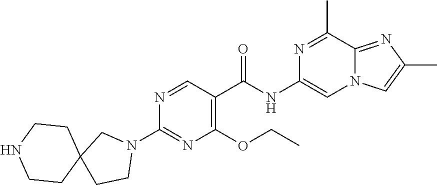 [SMILES: CCOc1nc(N2CCC3(CCNCC3)C2)ncc1C(=O)Nc1cn2cc(C)nc2c(C)n1] | 451.3 |
| 89 | 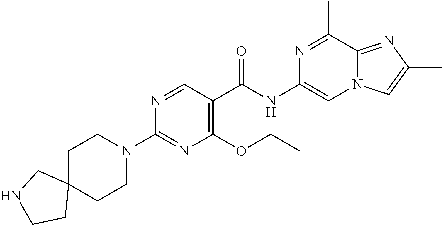 [SMILES: CCOc1nc(N2CCC3(CCNC3)CC2)ncc1C(=O)Nc1cn2cc(C)nc2c(C)n1] | 451.3 |
| 90 | 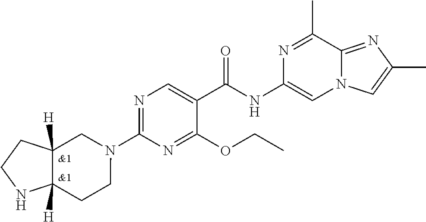 [SMILES: CCOc1nc(N2CC[C@@H]3NCC[C@@H]3C2)ncc1C(=O)Nc1cn2cc(C)nc2c(C)n1] | 437.2 |
| 91 | 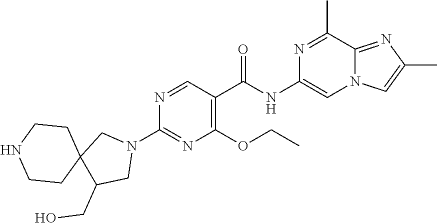 [SMILES: CCOc1nc(N2CC(CO)C3(CCNCC3)C2)ncc1C(=O)Nc1cn2cc(C)nc2c(C)n1] | 481.4 |
| 92 | 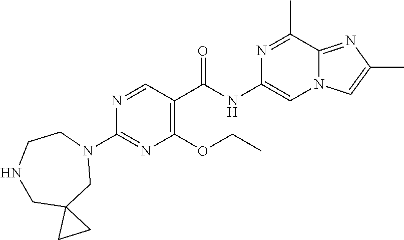 [SMILES: CCOc1nc(N2CCNCC3(CC3)C2)ncc1C(=O)Nc1cn2cc(C)nc2c(C)n1] | 437.4 |
| 93 | 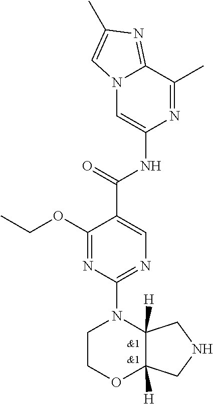 [SMILES: CCOc1nc(N2CCO[C@H]3CNC[C@H]32)ncc1C(=O)Nc1cn2cc(C)nc2c(C)n1] | 439.4 |
| 94 | 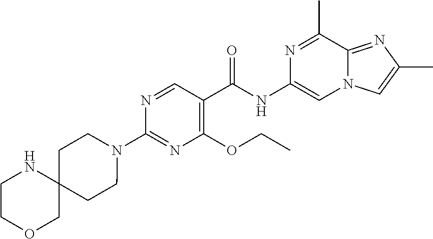 [SMILES: CCOc1nc(N2CCC3(CC2)COCCN3)ncc1C(=O)Nc1cn2cc(C)nc2c(C)n1] | 467.3 |
| 95 | 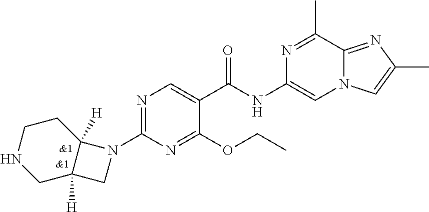 [SMILES: CCOc1nc(N2C[C@H]3CNCC[C@H]32)ncc1C(=O)Nc1cn2cc(C)nc2c(C)n1] | 423.2 |
| 96 | 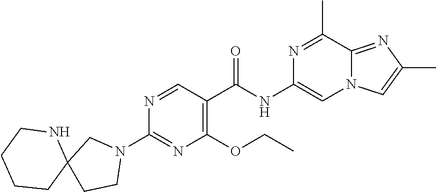 [SMILES: CCOc1nc(N2CCC3(CCCCN3)C2)ncc1C(=O)Nc1cn2cc(C)nc2c(C)n1] | 451.2 |
| 97 | 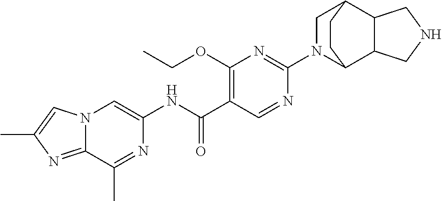 [SMILES: CCOc1nc(N2CC3CCC2C2CNCC32)ncc1C(=O)Nc1cn2cc(C)nc2c(C)n1] | 463.3 |
| 98 | 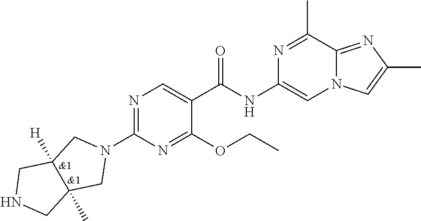 [SMILES: CCOc1nc(N2C[C@@H]3CNC[C@]3(C)C2)ncc1C(=O)Nc1cn2cc(C)nc2c(C)n1] | 436.2 |
| 99 | 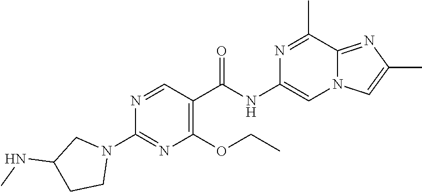 | 411.4 |
| 100 | 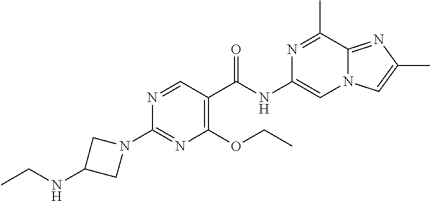 [SMILES: CCNC1CN(c2ncc(C(=O)Nc3cn4cc(C)nc4c(C)n3)c(OCC)n2)C1] | 411.3 |
| 101 | 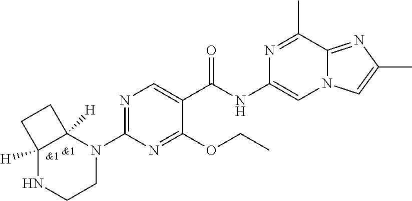 [SMILES: CCOc1nc(N2CCN[C@H]3CC[C@H]32)ncc1C(=O)Nc1cn2cc(C)nc2c(C)n1] | 423.2 |
| 102 | 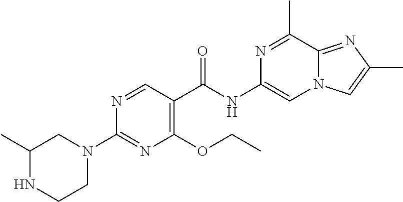 | 411.2 |
| 103 | 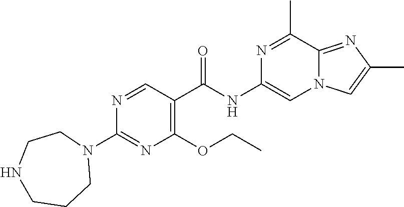 [SMILES: CCOc1nc(N2CCCNCC2)ncc1C(=O)Nc1cn2cc(C)nc2c(C)n1] | 411.2 |
| 104 | 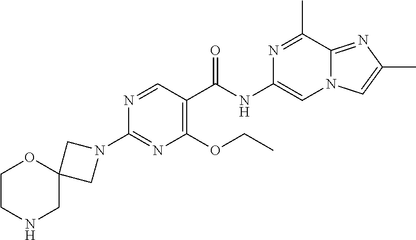 [SMILES: CCOc1nc(N2CC3(CNCCO3)C2)ncc1C(=O)Nc1cn2cc(C)nc2c(C)n1] | 439.2 |
| 105 | 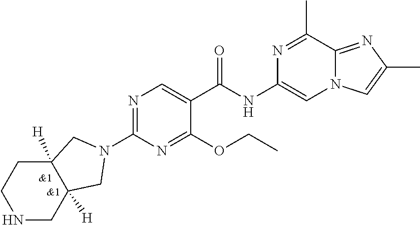 [SMILES: CCOc1nc(N2C[C@H]3CNCC[C@H]3C2)ncc1C(=O)Nc1cn2cc(C)nc2c(C)n1] | 437.1 |
| 106 | 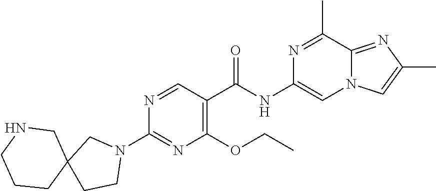 [SMILES: CCOc1nc(N2CCC3(CCCNC3)C2)ncc1C(=O)Nc1cn2cc(C)nc2c(C)n1] | 451.2 |

### Example 30. Parallel synthesis of Compound 107-248

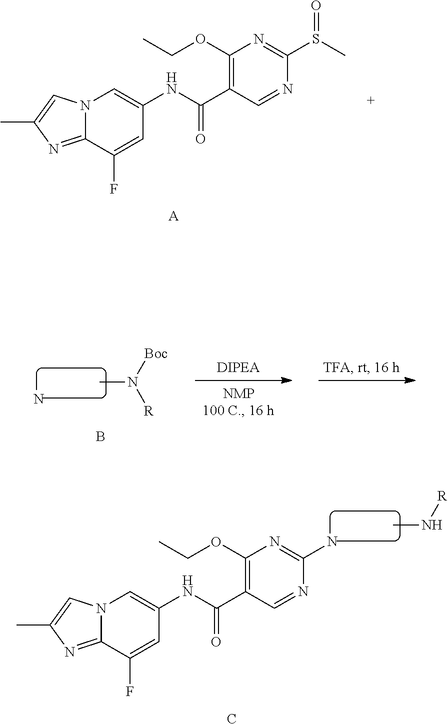

To a mixture of 1 eq. of ArSOMe (A) and 2 eq. amine (B) in Iml NMP was added 5.0 eq.* DIPEA. The reaction mixture was stirred at 100° C. for 16 hours. After cooling to room temperature, the solvent was evaporated and 1 ml of cleavage cocktail ** was added. The mixture was left to react for 4 h and evaporated to dryness. The residue was dissolved in DMSO and subjected to HPLC purification ***

*In case of using Reagent 1 and/or Reagent 2 as salt, an additional amount DIPEA (1.1 eq. for each eq. of acid) was added to the reaction mixture to convert the reagent into a free form.

** The cleavage cocktail was prepared by mixing TFA (92.5% v/v), water (5% v/v) and TIPS (2.5% v/v).

*** Resulting solution was purified by HPLC (Deionized Water (phase A) and HPLC-grade Acetonitrile (phase B) were used as an eluent to obtained final compound (D). In most cases, TFA was used as an additive.

Instrument: Agilent 1260 Infinity systems equipped with DAD and mass-detector

Column: Waters Sunfire C18 OBD Prep Column, 100 A, 5 μm, 19 mm×100 mm with SunFire C18 Prep Guard Cartridge, 100 A, 10 μm, 19 mm×10 mm.

| Compound | | ESI-MS |
|---|---|---|
| No | Structure | (M + H)⁺ |
| 107 | 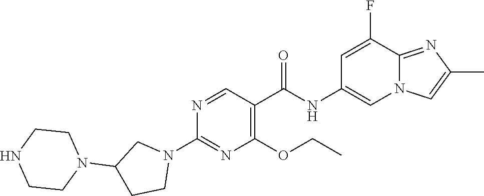 [SMILES: CCOc1nc(N2CCC(N3CCNCC3)C2)ncc1C(=O)Nc1cc(F)c2nc(C)cn2c1] | 469.0 |
| 108 | 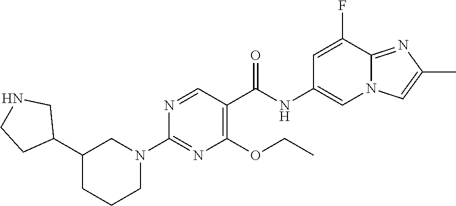 [SMILES: CCOc1nc(N2CCCC(C3CCNC3)C2)ncc1C(=O)Nc1cc(F)c2nc(C)cn2c1] | 468.4 |
| 109 | 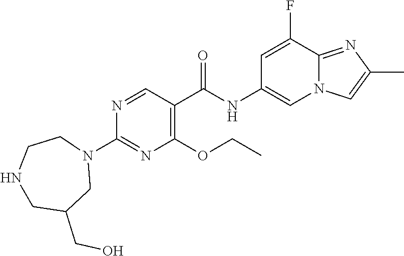 [SMILES: CCOc1nc(N2CCNCC(CO)C2)ncc1C(=O)Nc1cc(F)c2nc(C)cn2c1] | 444.2 |
| 110 | 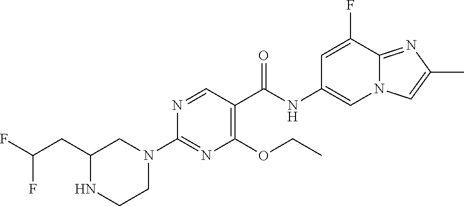 [SMILES: CCOc1nc(N2CCNC(CC(F)F)C2)ncc1C(=O)Nc1cc(F)c2nc(C)cn2c1] | 464.2 |
| 111 | 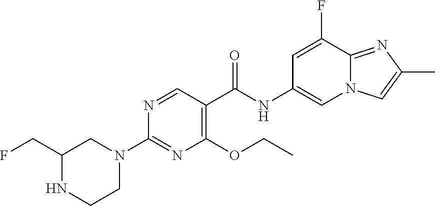 [SMILES: CCOc1nc(N2CCNC(CF)C2)ncc1C(=O)Nc1cc(F)c2nc(C)cn2c1] | 432.2 |
| 112 | 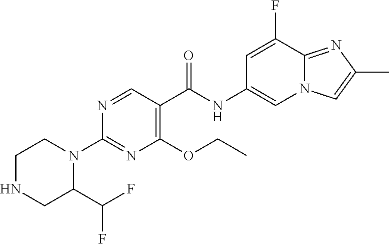 [SMILES: CCOc1nc(N2CCNCC2C(F)F)ncc1C(=O)Nc1cc(F)c2nc(C)cn2c1] | 450.1 |
| 113 | 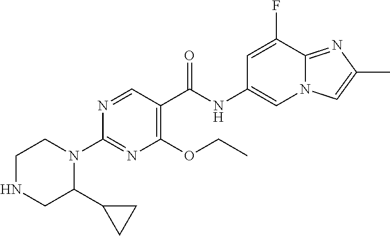 [SMILES: CCOc1nc(N2CCNCC2C2CC2)ncc1C(=O)Nc1cc(F)c2nc(C)cn2c1] | 440.1 |
| 114 | 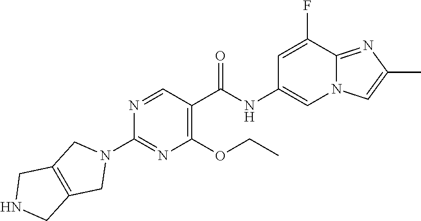 [SMILES: CCOc1nc(N2CC3=C(CNC3)C2)ncc1C(=O)Nc1cc(F)c2nc(C)cn2c1] | 424.2 |
| 115 | 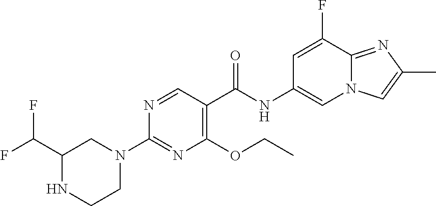 [SMILES: CCOc1nc(N2CCNC(C(F)F)C2)ncc1C(=O)Nc1cc(F)c2nc(C)cn2c1] | 450.2 |
| 116 | 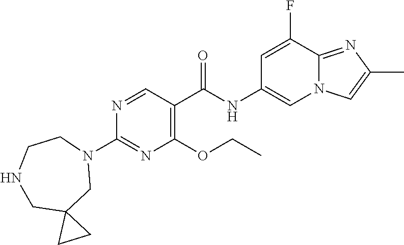 [SMILES: CCOc1nc(N2CCNCC3(CC3)C2)ncc1C(=O)Nc1cc(F)c2nc(C)cn2c1] | 440.2 |
| 117 | 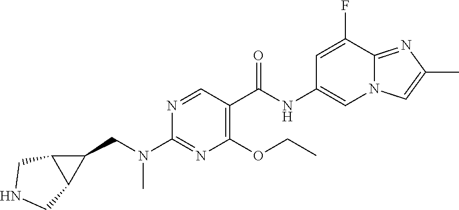 [SMILES: CCOc1nc(N(C)C[C@H]2[C@@H]3CNC[C@@H]32)ncc1C(=O)Nc1cc(F)c2nc(C)cn2c1] | 440.2 |
| 118 | 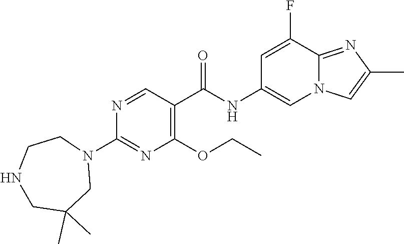 [SMILES: CCOc1nc(N2CCNCC(C)(C)C2)ncc1C(=O)Nc1cc(F)c2nc(C)cn2c1] | 442.2 |
| 119 | 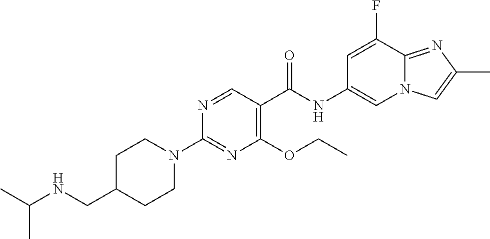 [SMILES: CCOc1nc(N2CCC(CNC(C)C)CC2)ncc1C(=O)Nc1cc(F)c2nc(C)cn2c1] | 470.2 |
| 120 | 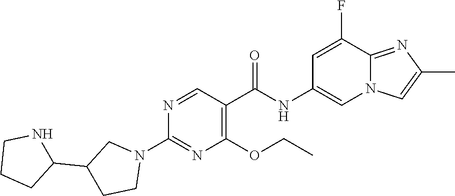 [SMILES: CCOc1nc(N2CCC(C3CCCN3)C2)ncc1C(=O)Nc1cc(F)c2nc(C)cn2c1] | 454.5 |
| 121 | 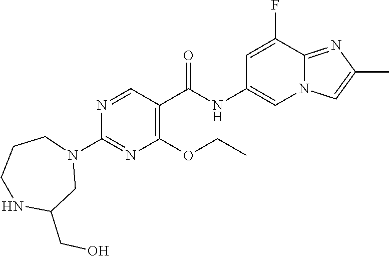 [SMILES: CCOc1nc(N2CCCNC(CO)C2)ncc1C(=O)Nc1cc(F)c2nc(C)cn2c1] | 444.2 |
| 122 | 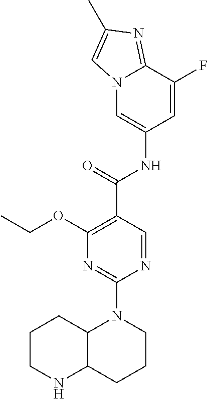 [SMILES: CCOc1nc(N2CCCC3NCCCC32)ncc1C(=O)Nc1cc(F)c2nc(C)cn2c1] | 454.2 |
| 123 | 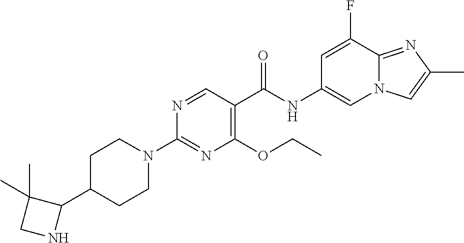 | 482.2 |
| 124 | 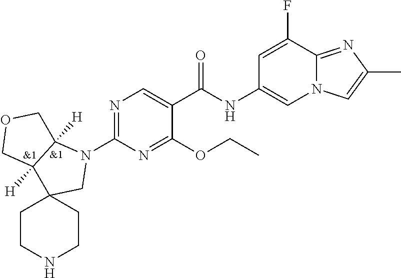 [SMILES: CCOc1nc(N2CC3(CCNCC3)[C@H]3COC[C@H]32)ncc1C(=O)Nc1cc(F)c2nc(C)cn2c1] | 496.4 |
| 125 | 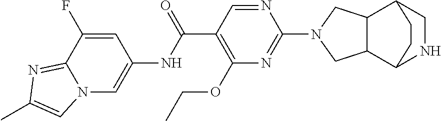 [SMILES: CCOc1nc(N2CC3C4CCC(NC4)C3C2)ncc1C(=O)Nc1cc(F)c2nc(C)cn2c1] | 466.2 |
| 126 | 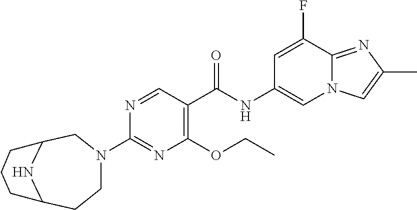 [SMILES: CCOc1nc(N2CCC3CCCC(C2)N3)ncc1C(=O)Nc1cc(F)c2nc(C)cn2c1] | 454.2 |
| 127 | 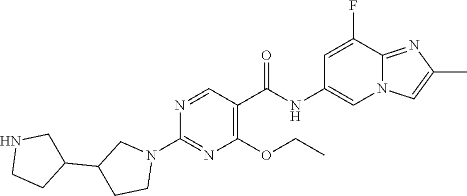 [SMILES: CCOc1nc(N2CCC(C3CCNC3)C2)ncc1C(=O)Nc1cc(F)c2nc(C)cn2c1] | 455.3 |
| 128 | 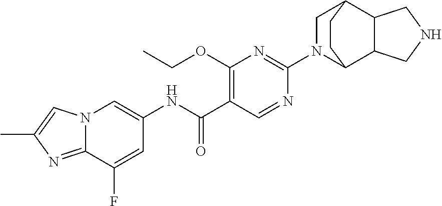 [SMILES: CCOc1nc(N2CC3CCC2C2CNCC32)ncc1C(=O)Nc1cc(F)c2nc(C)cn2c1] | 466.3 |
| 129 | 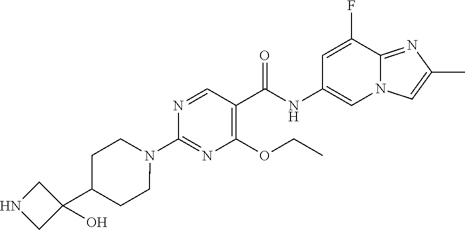 [SMILES: CCOc1nc(N2CCC(C3(O)CNC3)CC2)ncc1C(=O)Nc1cc(F)c2nc(C)cn2c1] | 470.0 |
| 130 | 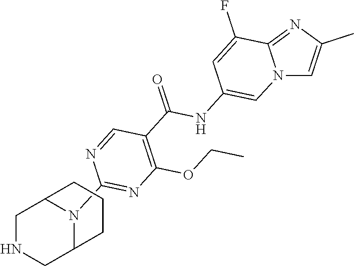 [SMILES: CCOc1nc(N2C3CCCC2CNC3)ncc1C(=O)Nc1cc(F)c2nc(C)cn2c1] | 440.2 |
| 131 | 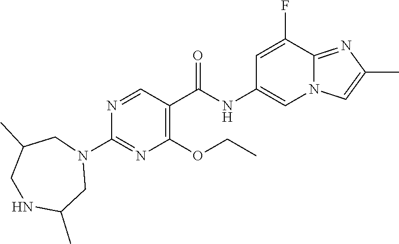 | 442.2 |
| 132 | 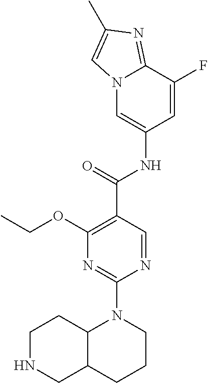 [SMILES: CCOc1nc(N2CCCC3CNCCC32)ncc1C(=O)Nc1cc(F)c2nc(C)cn2c1] | 454.2 |
| 133 | 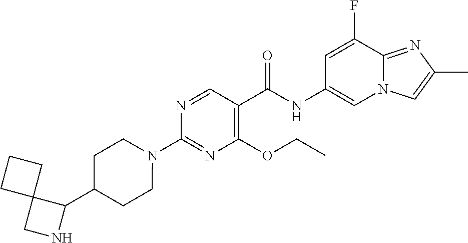 [SMILES: CCOc1nc(N2CCC(C3NCC34CCC4)CC2)ncc1C(=O)Nc1cc(F)c2nc(C)cn2c1] | 494.2 |
| 134 | 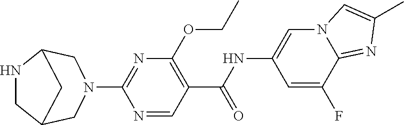 [SMILES: CCOc1nc(N2CC3CNC(C3)C2)ncc1C(=O)Nc1cc(F)c2nc(C)cn2c1] | 426.2 |
| 135 | 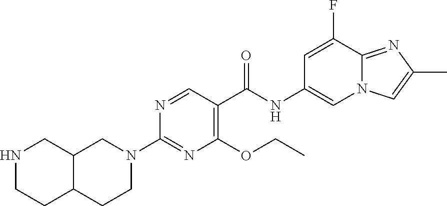 [SMILES: CCOc1nc(N2CCC3CCNCC3C2)ncc1C(=O)Nc1cc(F)c2nc(C)cn2c1] | 454.2 |
| 136 | 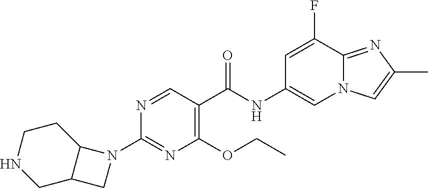 [SMILES: CCOc1nc(N2CC3CNCCC32)ncc1C(=O)Nc1cc(F)c2nc(C)cn2c1] | 426.1 |
| 137 | 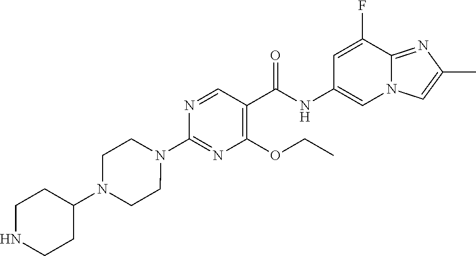 [SMILES: CCOc1nc(N2CCN(C3CCNCC3)CC2)ncc1C(=O)Nc1cc(F)c2nc(C)cn2c1] | 483.2 |
| 138 | 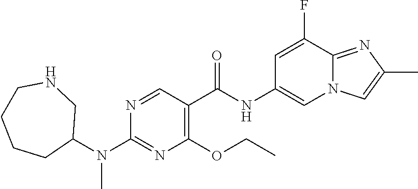 [SMILES: CCOc1nc(N(C)C2CCCCNC2)ncc1C(=O)Nc1cc(F)c2nc(C)cn2c1] | 442.2 |
| 139 | 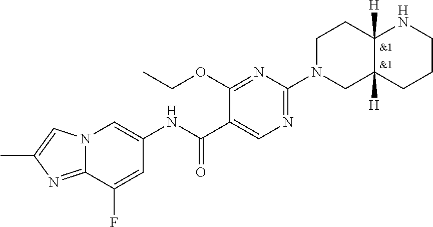 [SMILES: CCOc1nc(N2CC[C@@H]3NCCC[C@@H]3C2)ncc1C(=O)Nc1cc(F)c2nc(C)cn2c1] | 454.2 |
| 140 | 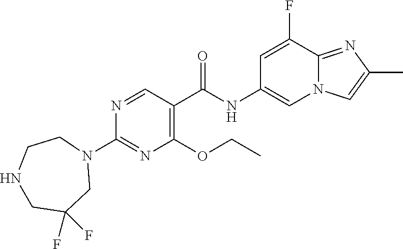 [SMILES: CCOc1nc(N2CCNCC(F)(F)C2)ncc1C(=O)Nc1cc(F)c2nc(C)cn2c1] | 450.2 |
| 141 | 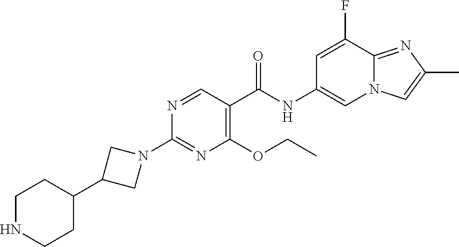 [SMILES: CCOc1nc(N2CC(C3CCNCC3)C2)ncc1C(=O)Nc1cc(F)c2nc(C)cn2c1] | 454.2 |
| 142 | 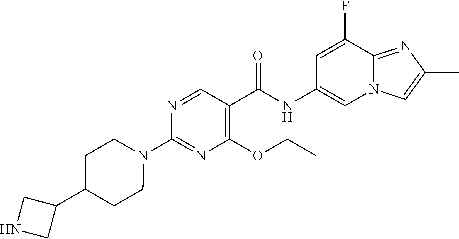 [SMILES: CCOc1nc(N2CCC(C3CNC3)CC2)ncc1C(=O)Nc1cc(F)c2nc(C)cn2c1] | 454.2 |
| 143 | 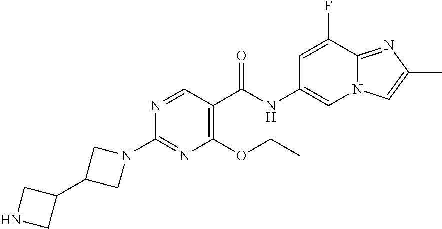 [SMILES: CCOc1nc(N2CC(C3CNC3)C2)ncc1C(=O)Nc1cc(F)c2nc(C)cn2c1] | 427.0 |
| 144 | 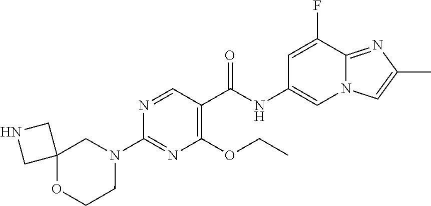 [SMILES: CCOc1nc(N2CCOC3(CNC3)C2)ncc1C(=O)Nc1cc(F)c2nc(C)cn2c1] | 442.2 |
| 145 | 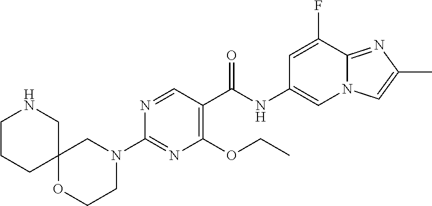 [SMILES: CCOc1nc(N2CCOC3(CCCNC3)C2)ncc1C(=O)Nc1cc(F)c2nc(C)cn2c1] | 470.1 |
| 146 | 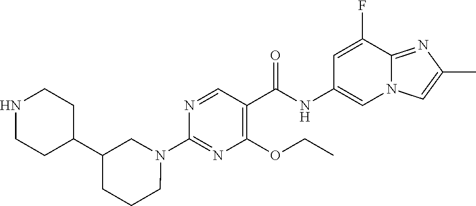 [SMILES: CCOc1nc(N2CCCC(C3CCNCC3)C2)ncc1C(=O)Nc1cc(F)c2nc(C)cn2c1] | 482.2 |
| 147 | 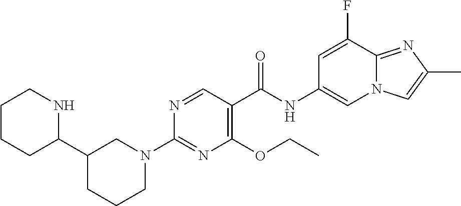 [SMILES: CCOc1nc(N2CCCC(C3CCCCN3)C2)ncc1C(=O)Nc1cc(F)c2nc(C)cn2c1] | 482.3 |
| 148 | 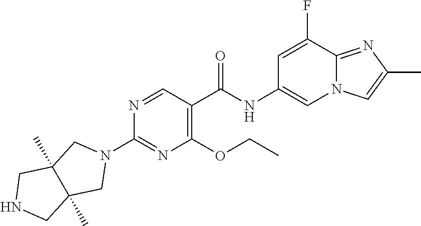 [SMILES: CCOc1nc(N2C[C@]3(C)CNC[C@]3(C)C2)ncc1C(=O)Nc1cc(F)c2nc(C)cn2c1] | 454.2 |
| 149 | 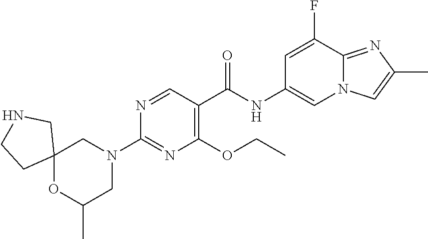 [SMILES: CCOc1nc(N2CC(C)OC3(CCNC3)C2)ncc1C(=O)Nc1cc(F)c2nc(C)cn2c1] | 470.2 |
| 150 | 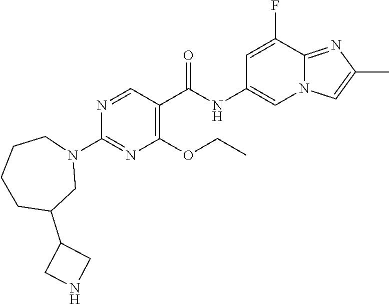 [SMILES: CCOc1nc(N2CCCCC(C3CNC3)C2)ncc1C(=O)Nc1cc(F)c2nc(C)cn2c1] | 468.2 |
| 151 | 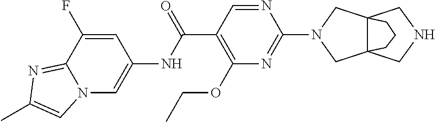 [SMILES: CCOc1nc(N2CC34CCCC3(CNC4)C2)ncc1C(=O)Nc1cc(F)c2nc(C)cn2c1] | 466.3 |
| 152 | 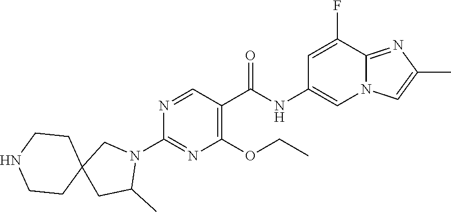 [SMILES: CCOc1nc(N2CC3(CCNCC3)CC2C)ncc1C(=O)Nc1cc(F)c2nc(C)cn2c1] | 468.2 |
| 153 | 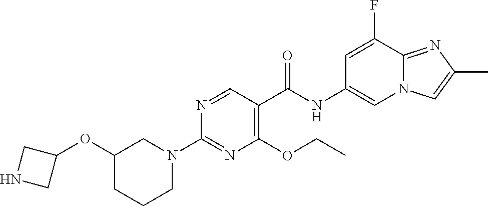 [SMILES: CCOc1nc(N2CCCC(OC3CNC3)C2)ncc1C(=O)Nc1cc(F)c2nc(C)cn2c1] | 470.2 |
| 154 | 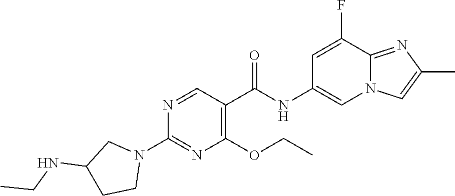 [SMILES: CCNC1CCN(c2ncc(C(=O)Nc3cc(F)c4nc(C)cn4c3)c(OCC)n2)C1] | 428.2 |
| 155 | 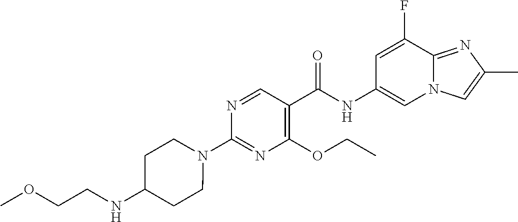 | 472.3 |
| 156 | 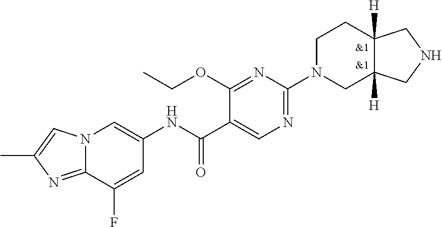 [SMILES: CCOc1nc(N2CC[C@@H]3CNC[C@@H]3C2)ncc1C(=O)Nc1cc(F)c2nc(C)cn2c1] | 440.2 |
| 157 | 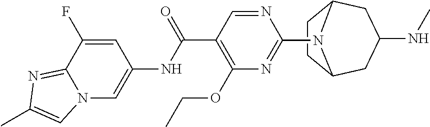 | 454.2 |
| 158 | 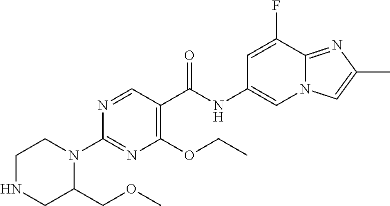 [SMILES: CCOc1nc(N2CCNCC2COC)ncc1C(=O)Nc1cc(F)c2nc(C)cn2c1] | 445.2 |
| 159 | 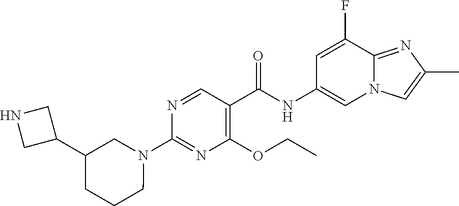 [SMILES: CCOc1nc(N2CCCC(C3CNC3)C2)ncc1C(=O)Nc1cc(F)c2nc(C)cn2c1] | 454.2 |
| 160 | 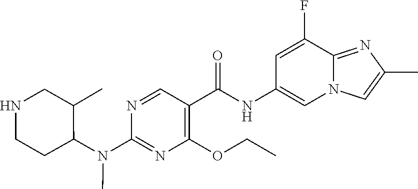 [SMILES: CCOc1nc(N(C)C2CCNCC2C)ncc1C(=O)Nc1cc(F)c2nc(C)cn2c1] | 442.0 |
| 161 | 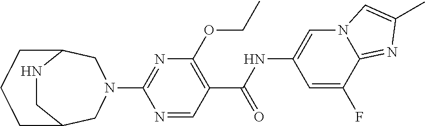 [SMILES: CCOc1nc(N2CC3CCCC(C2)NC3)ncc1C(=O)Nc1cc(F)c2nc(C)cn2c1] | 454.2 |
| 162 | 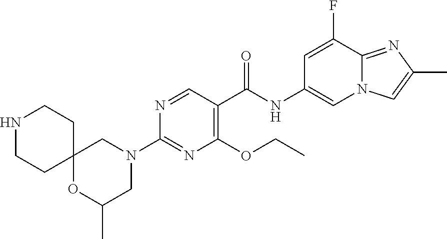 [SMILES: CCOc1nc(N2CC(C)OC3(CCNCC3)C2)ncc1C(=O)Nc1cc(F)c2nc(C)cn2c1] | 484.2 |
| 163 | 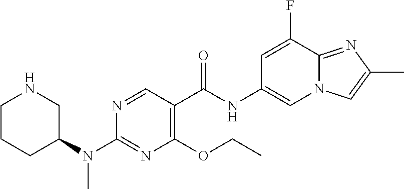 [SMILES: CCOc1nc(N(C)[C@H]2CCCNC2)ncc1C(=O)Nc1cc(F)c2nc(C)cn2c1] | 428.2 |
| 164 | 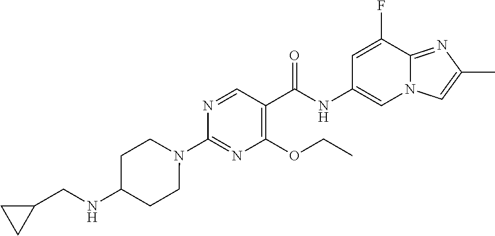 [SMILES: CCOc1nc(N2CCC(NCC3CC3)CC2)ncc1C(=O)Nc1cc(F)c2nc(C)cn2c1] | 468.2 |
| 165 | 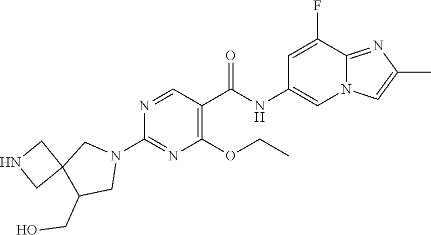 [SMILES: CCOc1nc(N2CC(CO)C3(CNC3)C2)ncc1C(=O)Nc1cc(F)c2nc(C)cn2c1] | 456.2 |
| 166 | 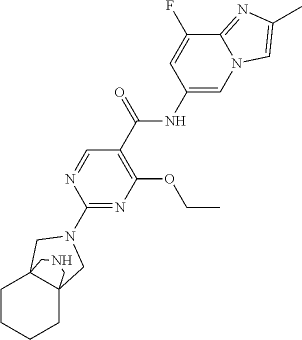 [SMILES: CCOc1nc(N2CC34CCCCC3(CNC4)C2)ncc1C(=O)Nc1cc(F)c2nc(C)cn2c1] | 480.4 |
| 167 | 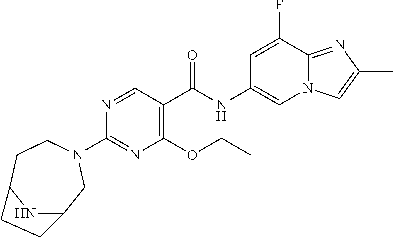 [SMILES: CCOc1nc(N2CCC3CCC(C2)N3)ncc1C(=O)Nc1cc(F)c2nc(C)cn2c1] | 440.4 |
| 168 | 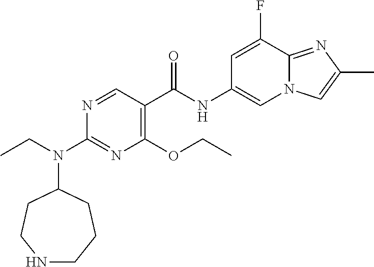 [SMILES: CCOc1nc(N(CC)C2CCCNCC2)ncc1C(=O)Nc1cc(F)c2nc(C)cn2c1] | 456.4 |
| 169 | 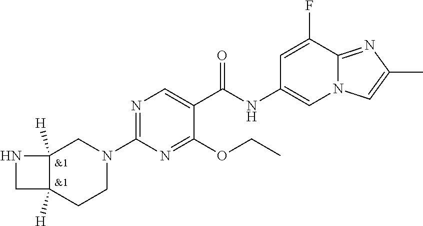 [SMILES: CCOc1nc(N2CC[C@H]3CN[C@H]3C2)ncc1C(=O)Nc1cc(F)c2nc(C)cn2c1] | 426.2 |
| 170 | 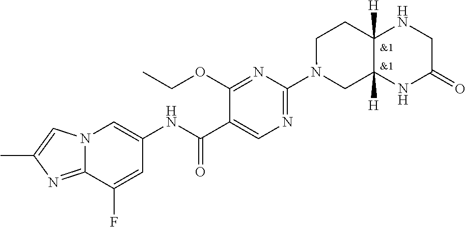 [SMILES: CCOc1nc(N2CC[C@@H]3NCC(=O)N[C@@H]3C2)ncc1C(=O)Nc1cc(F)c2nc(C)cn2c1] | 469.2 |
| 171 | 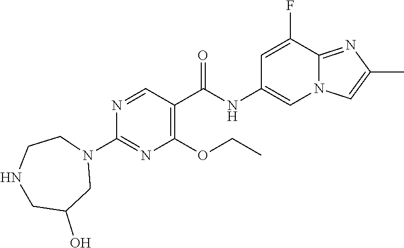 [SMILES: CCOc1nc(N2CCNCC(O)C2)ncc1C(=O)Nc1cc(F)c2nc(C)cn2c1] | 430.4 |
| 172 | 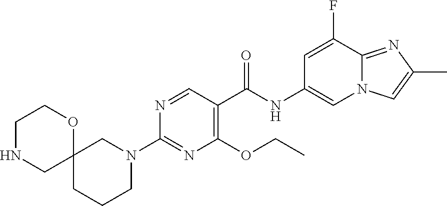 [SMILES: CCOc1nc(N2CCCC3(CNCCO3)C2)ncc1C(=O)Nc1cc(F)c2nc(C)cn2c1] | 470.1 |
| 173 | 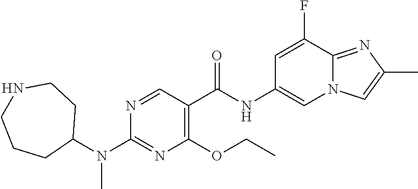 [SMILES: CCOc1nc(N(C)C2CCCNCC2)ncc1C(=O)Nc1cc(F)c2nc(C)cn2c1] | 442.2 |
| 174 | 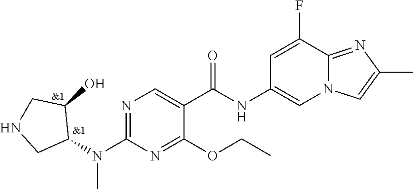 [SMILES: CCOc1nc(N(C)[C@@H]2CNC[C@H]2O)ncc1C(=O)Nc1cc(F)c2nc(C)cn2c1] | 430.2 |
| 175 | 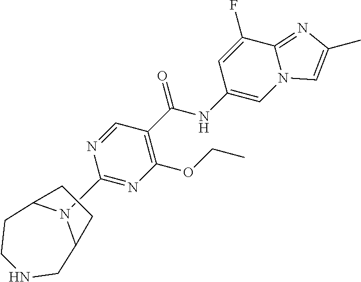 [SMILES: CCOc1nc(N2C3CCNCC2CC3)ncc1C(=O)Nc1cc(F)c2nc(C)cn2c1] | 440.4 |
| 176 | 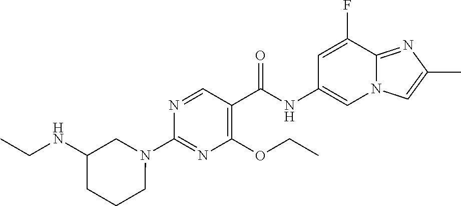 [SMILES: CCNC1CCCN(c2ncc(C(=O)Nc3cc(F)c4nc(C)cn4c3)c(OCC)n2)C1] | 442.2 |
| 177 | 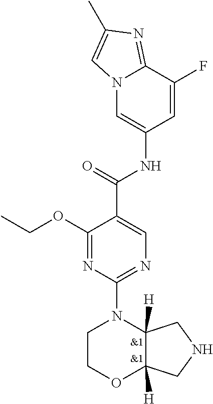 [SMILES: CCOc1nc(N2CCO[C@H]3CNC[C@H]32)ncc1C(=O)Nc1cc(F)c2nc(C)cn2c1] | 442.0 |
| 178 | 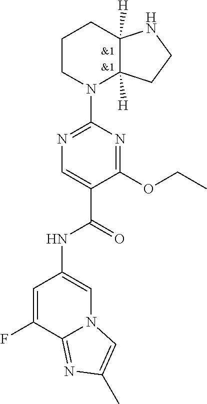 [SMILES: CCOc1nc(N2CCC[C@H]3NCC[C@H]32)ncc1C(=O)Nc1cc(F)c2nc(C)cn2c1] | 440.2 |
| 179 | 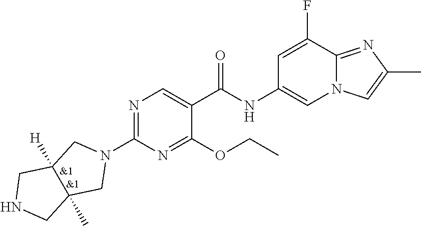 [SMILES: CCOc1nc(N2C[C@@H]3CNC[C@]3(C)C2)ncc1C(=O)Nc1cc(F)c2nc(C)cn2c1] | 440.4 |
| 180 | 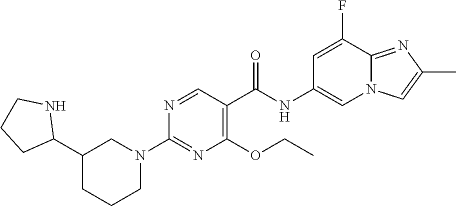 [SMILES: CCOc1nc(N2CCCC(C3CCCN3)C2)ncc1C(=O)Nc1cc(F)c2nc(C)cn2c1] | 468.2 |
| 181 | 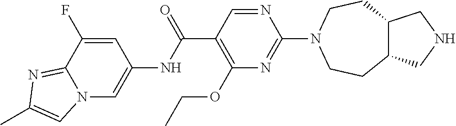 [SMILES: CCOc1nc(N2CC[C@@H]3CNC[C@@H]3CC2)ncc1C(=O)Nc1cc(F)c2nc(C)cn2c1] | 454.2 |
| 182 | 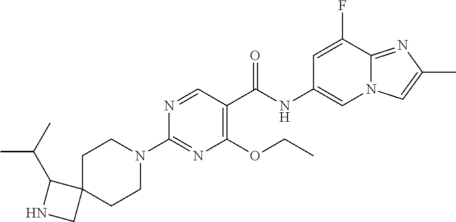 [SMILES: CCOc1nc(N2CCC3(CC2)CNC3C(C)C)ncc1C(=O)Nc1cc(F)c2nc(C)cn2c1] | 482.4 |
| 183 | 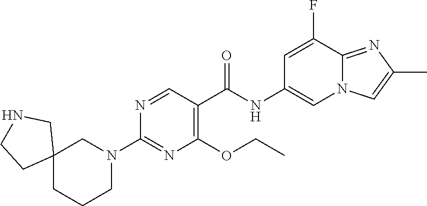 [SMILES: CCOc1nc(N2CCCC3(CCNC3)C2)ncc1C(=O)Nc1cc(F)c2nc(C)cn2c1] | 454.4 |
| 184 | 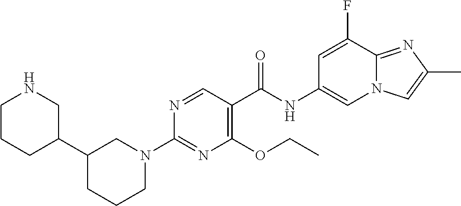 [SMILES: CCOc1nc(N2CCCC(C3CCCNC3)C2)ncc1C(=O)Nc1cc(F)c2nc(C)cn2c1] | 482.4 |
| 185 | 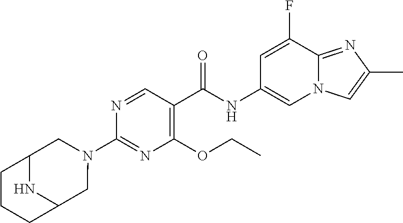 [SMILES: CCOc1nc(N2CC3CCCC(C2)N3)ncc1C(=O)Nc1cc(F)c2nc(C)cn2c1] | 440.2 |
| 186 | 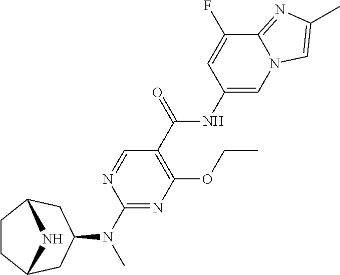 [SMILES: CCOc1nc(N(C)[C@@H]2C[C@H]3CC[C@@H](C2)N3)ncc1C(=O)Nc1cc(F)c2nc(C)cn2c1] | 454.4 |
| 187 | 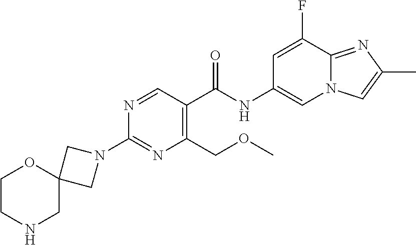 [SMILES: COCc1nc(N2CC3(CNCCO3)C2)ncc1C(=O)Nc1cc(F)c2nc(C)cn2c1] | 442.0 |
| 188 | 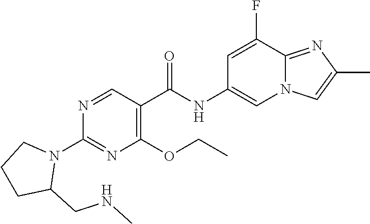 [SMILES: CCOc1nc(N2CCCC2CNC)ncc1C(=O)Nc1cc(F)c2nc(C)cn2c1] | 428.3 |
| 189 | 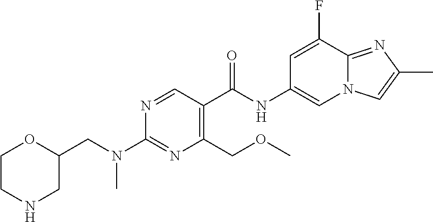 [SMILES: COCc1nc(N(C)CC2CNCCO2)ncc1C(=O)Nc1cc(F)c2nc(C)cn2c1] | 444.2 |
| 190 | 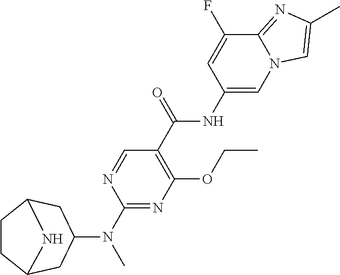 [SMILES: CCOc1nc(N(C)C2CC3CCC(C2)N3)ncc1C(=O)Nc1cc(F)c2nc(C)cn2c1] | 454.2 |
| 191 | 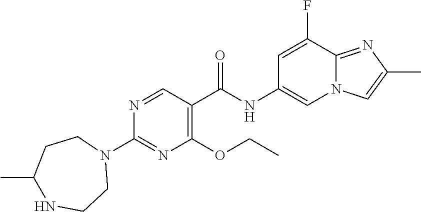 | 428.4 |
| 192 | 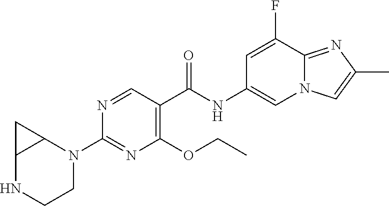 [SMILES: CCOc1nc(N2CCNC3CC32)ncc1C(=O)Nc1cc(F)c2nc(C)cn2c1] | 412.2 |
| 193 | 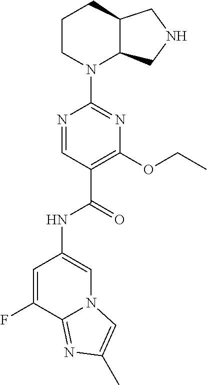 [SMILES: CCOc1nc(N2CCC[C@H]3CNC[C@H]32)ncc1C(=O)Nc1cc(F)c2nc(C)cn2c1] | 440.2 |
| 194 | 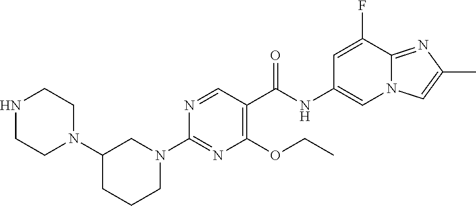 [SMILES: CCOc1nc(N2CCCC(N3CCNCC3)C2)ncc1C(=O)Nc1cc(F)c2nc(C)cn2c1] | 483.4 |
| 195 | 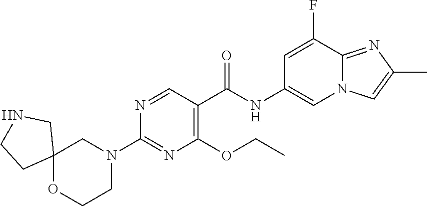 [SMILES: CCOc1nc(N2CCOC3(CCNC3)C2)ncc1C(=O)Nc1cc(F)c2nc(C)cn2c1] | 456.2 |
| 196 | 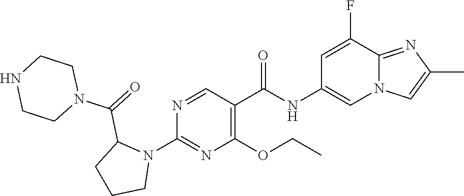 [SMILES: CCOc1nc(N2CCCC2C(=O)N2CCNCC2)ncc1C(=O)Nc1cc(F)c2nc(C)cn2c1] | 497.4 |
| 197 | 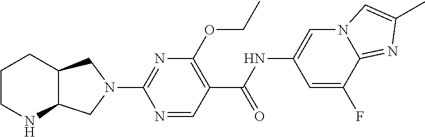 [SMILES: CCOc1nc(N2C[C@@H]3CCCN[C@@H]3C2)ncc1C(=O)Nc1cc(F)c2nc(C)cn2c1] | 440.2 |
| 198 | 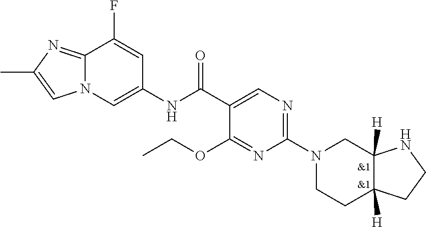 [SMILES: CCOc1nc(N2CC[C@H]3CCN[C@H]3C2)ncc1C(=O)Nc1cc(F)c2nc(C)cn2c1] | 440.2 |
| 199 | 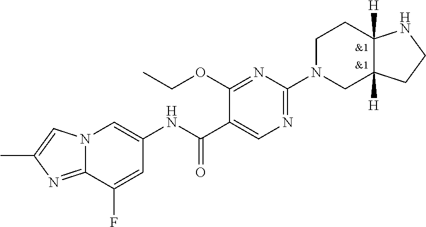 [SMILES: CCOc1nc(N2CC[C@@H]3NCC[C@@H]3C2)ncc1C(=O)Nc1cc(F)c2nc(C)cn2c1] | 440.2 |
| 200 | 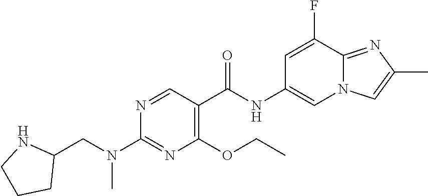 [SMILES: CCOc1nc(N(C)CC2CCCN2)ncc1C(=O)Nc1cc(F)c2nc(C)cn2c1] | 428.1 |
| 201 | 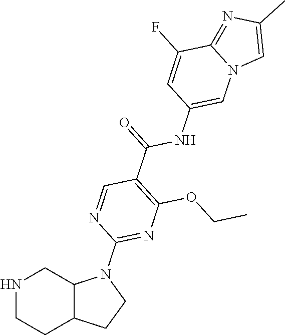 [SMILES: CCOc1nc(N2CCC3CCNCC32)ncc1C(=O)Nc1cc(F)c2nc(C)cn2c1] | 440.2 |
| 202 | 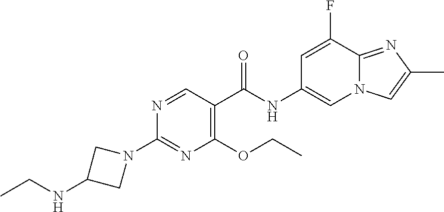 [SMILES: CCNC1CN(c2ncc(C(=O)Nc3cc(F)c4nc(C)cn4c3)c(OCC)n2)C1] | 414.2 |
| 203 | 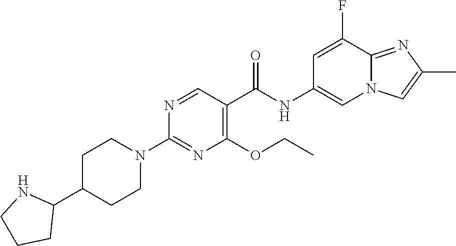 [SMILES: CCOc1nc(N2CCC(C3CCCN3)CC2)ncc1C(=O)Nc1cc(F)c2nc(C)cn2c1] | 468.2 |
| 204 | 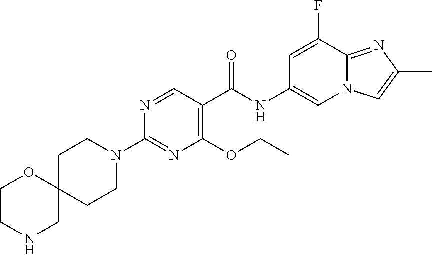 [SMILES: CCOc1nc(N2CCC3(CC2)CNCCO3)ncc1C(=O)Nc1cc(F)c2nc(C)cn2c1] | 470.4 |
| 205 | 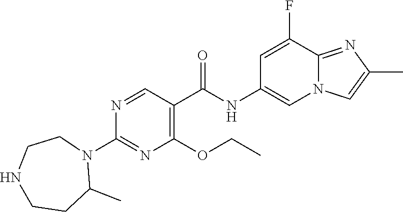 [SMILES: CCOc1nc(N2CCNCCC2C)ncc1C(=O)Nc1cc(F)c2nc(C)cn2c1] | 428.2 |
| 206 | 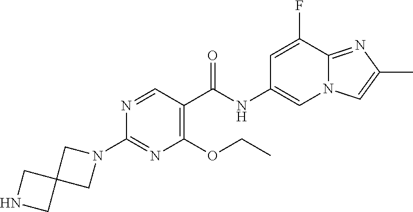 [SMILES: CCOc1nc(N2CC3(CNC3)C2)ncc1C(=O)Nc1cc(F)c2nc(C)cn2c1] | 412.2 |
| 207 | 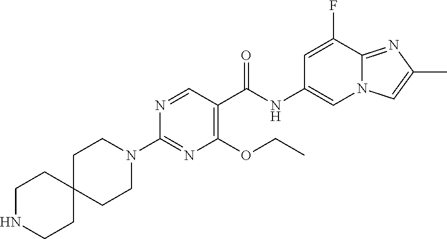 [SMILES: CCOc1nc(N2CCC3(CCNCC3)CC2)ncc1C(=O)Nc1cc(F)c2nc(C)cn2c1] | 468.2 |
| 208 | 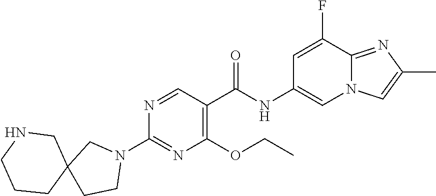 [SMILES: CCOc1nc(N2CCC3(CCCNC3)C2)ncc1C(=O)Nc1cc(F)c2nc(C)cn2c1] | 454.2 |
| 209 | 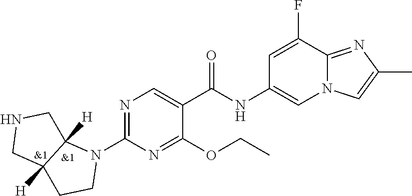 [SMILES: CCOc1nc(N2CC[C@@H]3CNC[C@@H]32)ncc1C(=O)Nc1cc(F)c2nc(C)cn2c1] | 426.1 |
| 210 | 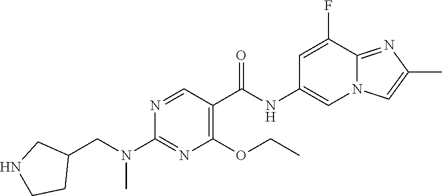 [SMILES: CCOc1nc(N(C)CC2CCNC2)ncc1C(=O)Nc1cc(F)c2nc(C)cn2c1] | 428.4 |
| 211 | 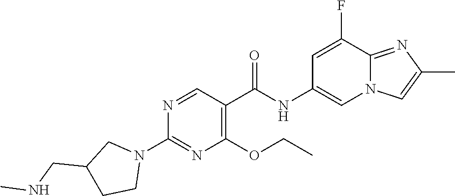 | 428.4 |
| 212 | 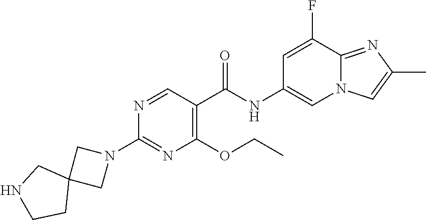 [SMILES: CCOc1nc(N2CC3(CCNC3)C2)ncc1C(=O)Nc1cc(F)c2nc(C)cn2c1] | 426.2 |
| 213 | 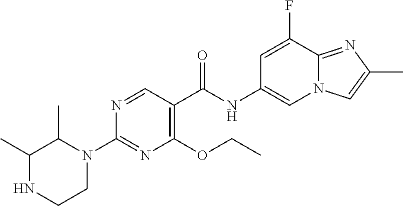 | 428.2 |
| 214 | 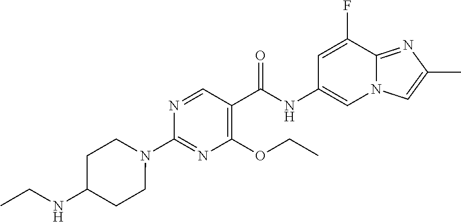 [SMILES: CCNC1CCN(c2ncc(C(=O)Nc3cc(F)c4nc(C)cn4c3)c(OCC)n2)CC1] | 442.2 |
| 215 | 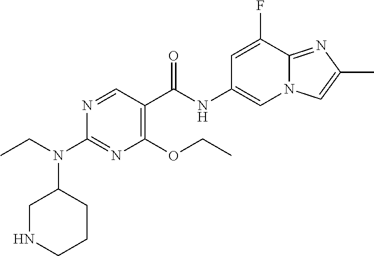 [SMILES: CCOc1nc(N(CC)C2CCCNC2)ncc1C(=O)Nc1cc(F)c2nc(C)cn2c1] | 442.4 |
| 216 | 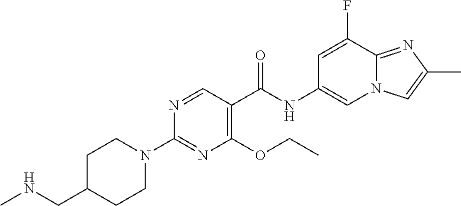 | 442.4 |
| 217 | 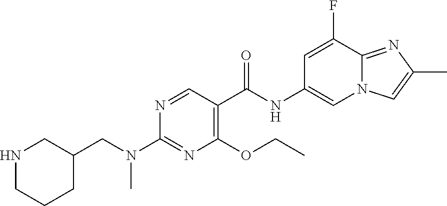 [SMILES: CCOc1nc(N(C)CC2CCCNC2)ncc1C(=O)Nc1cc(F)c2nc(C)cn2c1] | 442.4 |
| 218 | 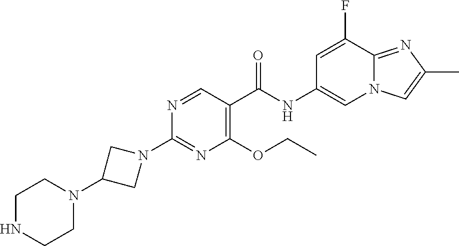 [SMILES: CCOc1nc(N2CC(N3CCNCC3)C2)ncc1C(=O)Nc1cc(F)c2nc(C)cn2c1] | 455.1 |
| 219 | 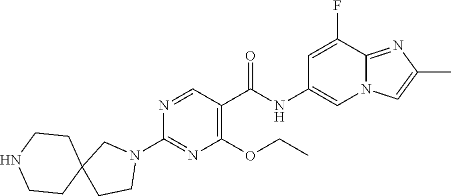 [SMILES: CCOc1nc(N2CCC3(CCNCC3)C2)ncc1C(=O)Nc1cc(F)c2nc(C)cn2c1] | 454.2 |
| 220 | 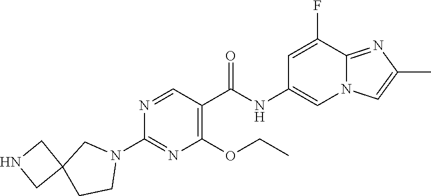 [SMILES: CCOc1nc(N2CCC3(CNC3)C2)ncc1C(=O)Nc1cc(F)c2nc(C)cn2c1] | 426.2 |
| 221 | 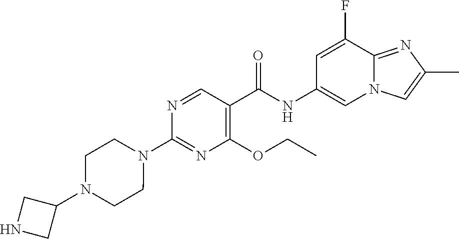 [SMILES: CCOc1nc(N2CCN(C3CNC3)CC2)ncc1C(=O)Nc1cc(F)c2nc(C)cn2c1] | 455.1 |
| 222 | 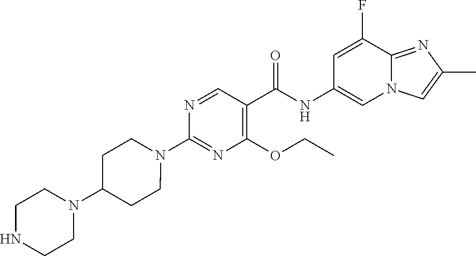 [SMILES: CCOc1nc(N2CCC(N3CCNCC3)CC2)ncc1C(=O)Nc1cc(F)c2nc(C)cn2c1] | 483.2 |
| 223 | 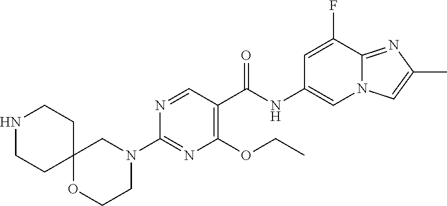 [SMILES: CCOc1nc(N2CCOC3(CCNCC3)C2)ncc1C(=O)Nc1cc(F)c2nc(C)cn2c1] | 470.0 |
| 224 | 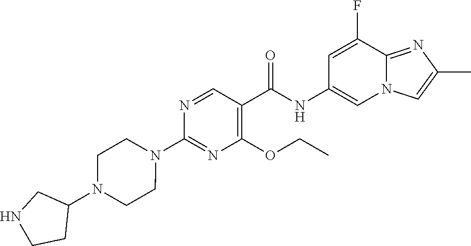 [SMILES: CCOc1nc(N2CCN(C3CCNC3)CC2)ncc1C(=O)Nc1cc(F)c2nc(C)cn2c1] | 469.2 |
| 225 | 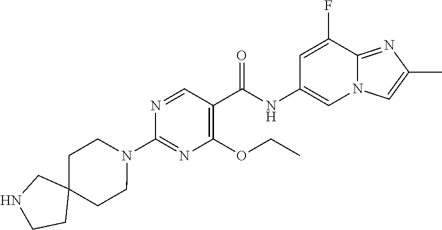 [SMILES: CCOc1nc(N2CCC3(CCNC3)CC2)ncc1C(=O)Nc1cc(F)c2nc(C)cn2c1] | 454.2 |
| 226 | 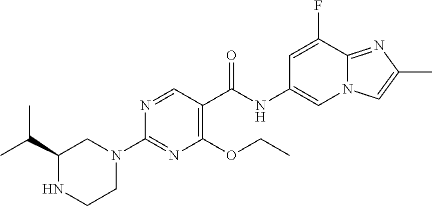 [SMILES: CCOc1nc(N2CCN[C@@H](C(C)C)C2)ncc1C(=O)Nc1cc(F)c2nc(C)cn2c1] | 442.4 |
| 227 | 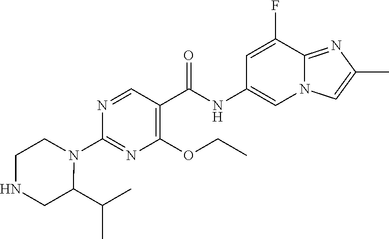 [SMILES: CCOc1nc(N2CCNCC2C(C)C)ncc1C(=O)Nc1cc(F)c2nc(C)cn2c1] | 442.2 |
| 228 | 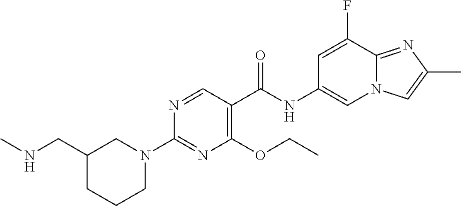 | 442.2 |
| 229 | 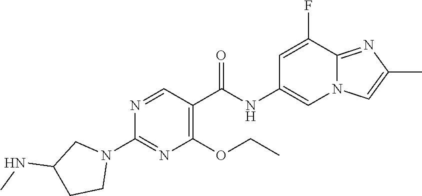 | 414.2 |
| 230 | 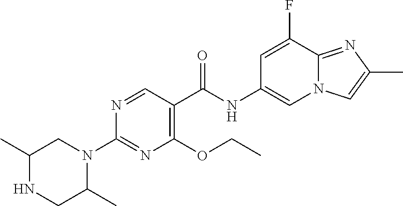 | 428.4 |
| 231 | 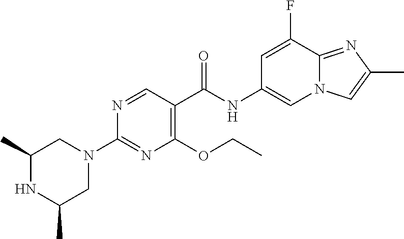 | 428.2 |
| 232 | 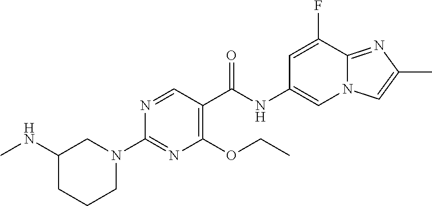 | 428.2 |
| 233 | 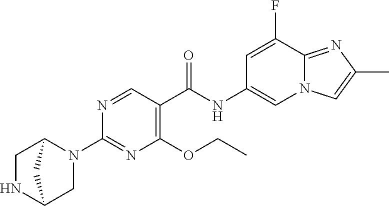 [SMILES: CCOc1nc(N2C[C@@H]3C[C@H]2CN3)ncc1C(=O)Nc1cc(F)c2nc(C)cn2c1] | 412.2 |
| 234 | 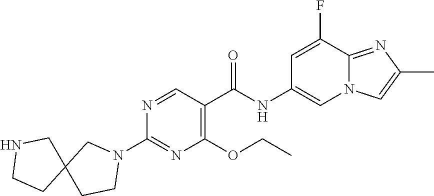 [SMILES: CCOc1nc(N2CCC3(CCNC3)C2)ncc1C(=O)Nc1cc(F)c2nc(C)cn2c1] | 440.2 |
| 235 | 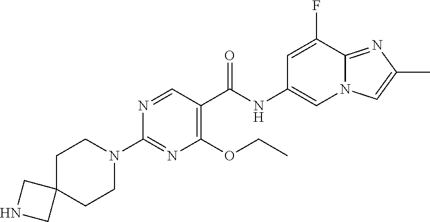 [SMILES: CCOc1nc(N2CCC3(CC2)CNC3)ncc1C(=O)Nc1cc(F)c2nc(C)cn2c1] | 440.2 |
| 236 | 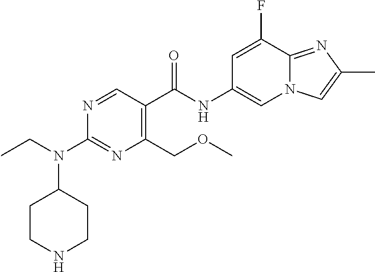 [SMILES: CCN(c1ncc(C(=O)Nc2cc(F)c3nc(C)cn3c2)c(COC)n1)C1CCNCC1] | 442.4 |
| 237 | 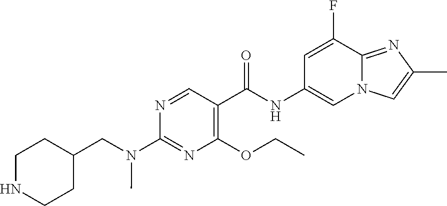 [SMILES: CCOc1nc(N(C)CC2CCNCC2)ncc1C(=O)Nc1cc(F)c2nc(C)cn2c1] | 442.2 |
| 238 | 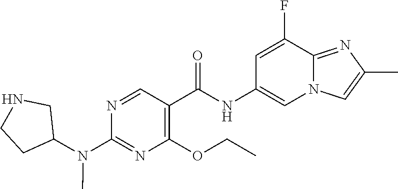 [SMILES: CCOc1nc(N(C)C2CCNC2)ncc1C(=O)Nc1cc(F)c2nc(C)cn2c1] | 414.2 |
| 239 | 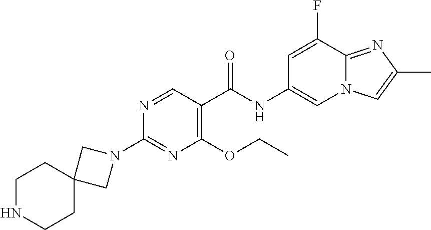 [SMILES: CCOc1nc(N2CC3(CCNCC3)C2)ncc1C(=O)Nc1cc(F)c2nc(C)cn2c1] | 440.2 |
| 240 | 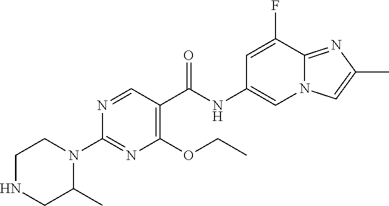 [SMILES: CCOc1nc(N2CCNCC2C)ncc1C(=O)Nc1cc(F)c2nc(C)cn2c1] | 414.2 |
| 241 | 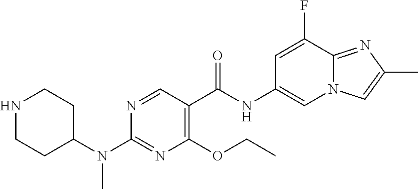 [SMILES: CCOc1nc(N(C)C2CCNCC2)ncc1C(=O)Nc1cc(F)c2nc(C)cn2c1] | 427.2 |
| 242 | 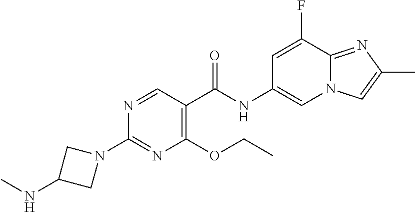 | 400.4 |
| 243 | 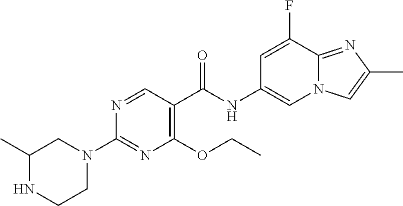 | 414.2 |
| 244 | 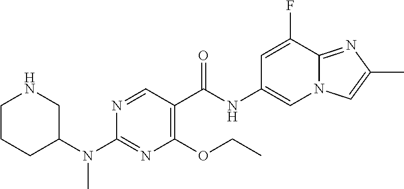 [SMILES: CCOc1nc(N(C)C2CCCNC2)ncc1C(=O)Nc1cc(F)c2nc(C)cn2c1] | 428.2 |
| 245 | 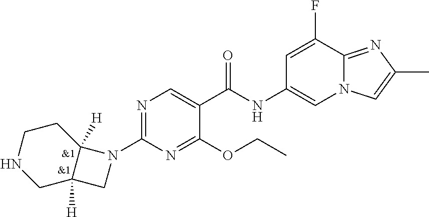 [SMILES: CCOc1nc(N2C[C@H]3CNCC[C@H]32)ncc1C(=O)Nc1cc(F)c2nc(C)cn2c1] | 426.2 |
| 246 | 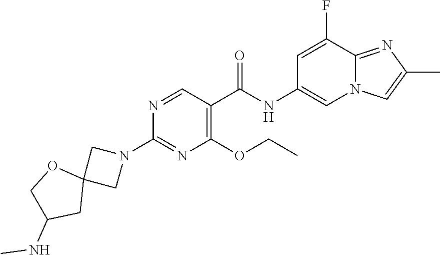 | 456.2 |
| 247 | 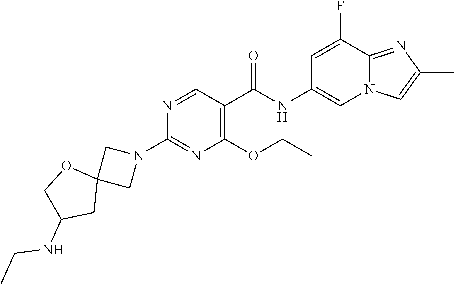 [SMILES: CCNC1COC2(C1)CN(c1ncc(C(=O)Nc3cc(F)c4nc(C)cn4c3)c(OCC)n1)C2] | 470.2 |
| 248 | 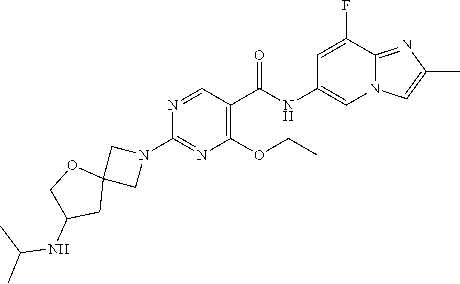 [SMILES: CCOc1nc(N2CC3(CC(NC(C)C)CO3)C2)ncc1C(=O)Nc1cc(F)c2nc(C)cn2c1] | 484.2 |
| 249 | 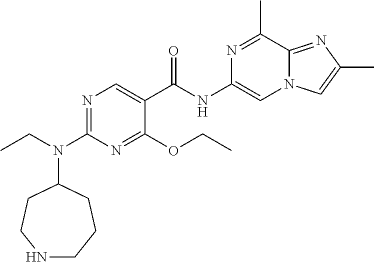 [SMILES: CCOc1nc(N(CC)C2CCCNCC2)ncc1C(=O)Nc1cn2cc(C)nc2c(C)n1] | 453.2 |

### Preparation of Starting Material: 2-(3-((tert-butoxycarbonyl) (methyl) amino) azetidin-1-yl)-4-ethoxypyrimidine-5-carboxylic acid

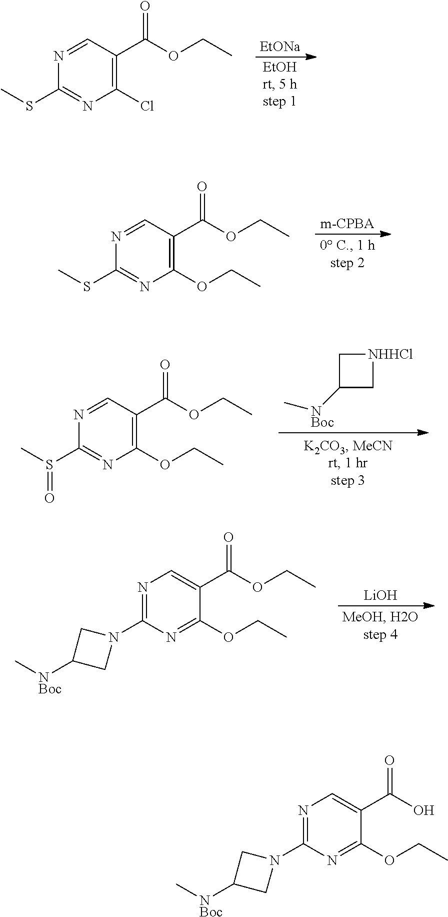

### Step 1: ethyl 4-ethoxy-2-(methylthio)pyrimidine-5-carboxylate

To a stirred solution of ethyl 4-chloro-2-(methylthio)pyrimidine-5-carboxylate (50 g, 215.0 mmol) in EtOH (500 mL) under argon atmosphere was added NaOEt (36.1 g, 645.0 mmol) and the mixture was stirred at rt for 5 h. The reaction mixture was filtered and filtrate was concentrated under reduce pressure. The residue was diluted with EtOAc and washed brine. The organic layer was concentrated and the residue was purified by silica gel column chromatography (PE/EA=4:1) to give ethyl 4-ethoxy-2-(methylthio)pyrimidine-5-carboxylate (43 g, Y: 83%) as a white solid.

### ESI-MS (M+H)⁺: 243.3.

### Step 2: ethyl 4-ethoxy-2-(methylsulfinyl)pyrimidine-5-carboxylate

To a stirred solution of ethyl 4-ethoxy-2-(methylthio)pyrimidine-5-carboxylate (20 g, 93.0 mmol) in DCM (200 mL) was added m-CPBA (16 g, 93.0 mmol) at 0° C. The mixture was warmed to rt and stirred for 1 h. The reaction mixture was diluted with DCM (200 mL) and quenched with sat. Na₂SO₃ (100 mL). The organic phase was washed with sat. sodium carbonate (200 mL), dried over sodium sulfate. After concentration under reduced pressure, ethyl 4-ethoxy-2-(methylsulfinyl)pyrimidine-5-carboxylate (20 g, Y: 93%) was obtained as a white solid, which was used to next step without further purification. ESI-MS (M+H)⁺: 259.0. ¹H NMR (400 MHZ, CDCl₃)δ 9.06 (s, 1H), 4.66 (q, J=7.1 Hz, 2H), 4.41 (q, J=7.1 Hz, 2H), 2.98 (s, 3H), 1.48 (t, J=7.1 Hz, 3H), 1.41 (t, J=7.1 Hz, 3H).

### Step 3: synthesis of ethyl 2-(3-((tert-butoxycarbonyl) (methyl) amino) azetidin-1-yl)-4-ethoxypyrimidine-5-carboxylate

To a stirred solution of ethyl 4-ethoxy-2-(methylsulfinyl)pyrimidine-S-carboxylate (3 g, 11.61 mmol) in MeCN (80 mL) was added tert-butyl azetidin-3-yl(methyl) carbamate hydrochloride (5.17 g, 23.23 mmol) and potassium carbonate (3.21 g, 23.23 mmol). The mixture was stirred at rt for 1 h. After concentration, the residue was diluted with EA (200 mL), washed with water (150 mL), dried over sodium sulfate, filtered and concentrated. The residue was purified by silica gel column (PE:EA=2:1) to give title product (2 g, Y: 45.28%) as a white solid. ESI-MS (M+H)⁺: 381.2.

¹H NMR (400 MHZ, CDCl₃)δ 8.72 (s, 1H), 5.08 (br s, 1H), 4.46 (q, J=7.1 Hz, 2H), 4.36 (t, J=9.0 Hz, 2H), 4.29 (q, J=7.1 Hz, 2H), 4.20 (d, J=5.2 Hz, 2H), 3.01 (s, 3H), 1.47 (s, 9H), 1.42 (t, J=7.1 Hz, 3H), 1.34 (t, J=7.1 Hz, 3H).

### Step 4: synthesis of 2-(3-(tert-butoxycarbonyl) (methyl) amino) azetidin-1-yl)-1-ethoxypyrimidine-5-carboxylic acid

To a solution of ethyl 2-(3-((tert-butoxycarbonyl) (methyl) amino) azetidin-1-yl)-4-ethoxypyrimidine-5-carboxylate (2 g, 5.26 mmol) in MeOH (20 mL) and H₂O (20 mL) was added LiOH (630 mg, 26.28 mmol) at rt. The reaction mixture was stirred overnight at 50° C. After concentration, the residue was purified by reverse phase column (ACN in water, 50%,) to give title product (1 g, yield: 54%) as a white solid. ESI-MS (M+H)⁺: 353.1. ¹H NMR (400 MHz, CDCl₃) δ 8.65 (s, 1H), 5.19-4.70 (m, 3H), 4.60 (q, J=7.0 Hz, 2H), 4.54-4.39 (m, 2H), 3.00 (s, 3H), 1.52-1.41 (m, 12H).

### Preparation of Starting Material: 2-(3-((tert-butoxycarbonyl) (methyl) amino) pyrrolidin-1-yl)-4-ethoxypyrimidine-5-carboxylic acid

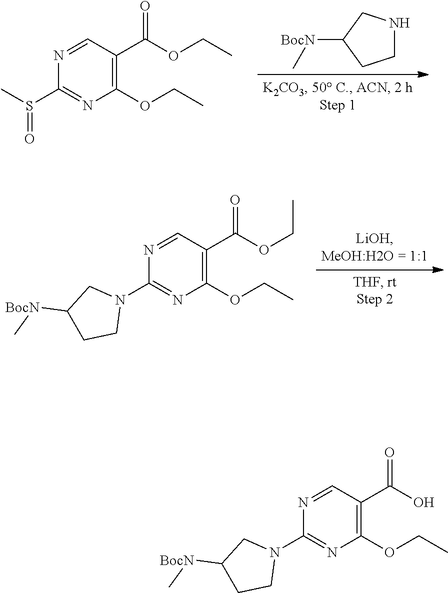

### Step 1: Preparation of ethyl 2-(3-((tert-butoxycarbonyl) (methyl) amino) pyrrolidin-1-yl)-4-ethoxypyrimidine-5-carboxylate

To a mixture of ethyl 4-ethoxy-2-(methylsulfinyl)pyrimidine-5-carboxylate (4.5 g, 22.50 mmol) and tert-butyl methyl(pyrrolidin-3-yl) carbamate (8.7 g, 33.75 mmol) in ACN (50 mL) was added K₂CO₃ (6.2 g, 45.00 mmol). The mixture was stirred at 50° C. for 2 h. The reaction was diluted with water (50 mL), extracted with EA (80 mL×3). The organic layer was washed with brine, dried over Na₂SO₄ and evaporated. The crude was purified by silica gel column chromatography (PE:EA=6:1) to give title product (6.5 g, yield 73.9%) as a white solid. ESI-MS (M+H)⁺: 395.0.

### Step 2: Preparation of 2-(3-(tert-butoxycarbonyl) (methyl) amino) pyrrolidin-1-yl)-1-ethoxypyrimidine-5-carboxylic acid

To a mixture of ethyl 2-(3-((tert-butoxycarbonyl) (methyl) amino) pyrrolidin-1-yl)-4-ethoxypyrimidine-5-carboxylate (6.5 g, 16.5 mmol) in MeOH (100 mL) and H₂O (100 mL) was added LiOH (3.38 g, 82.5 mmol). The reaction solution was stirred at RT for 1 h. After concentration to remove most of MeOH, the aqueous phase was adjusted to pH to 5, extracted with EA (80 mL×3). The organic layer was washed with brine, dried over Na₂SO₄, filtered and evaporated to give title product (5.5 g, yield 91.7%) as a white solid. ESI-MS (M+H)⁺: 367.3. ¹H NMR (400 MHz, DMSO-d₆) δ 8.59 (s, 1H), 4.79-4.53 (m, 1H), 4.42 (q, J=7.0 Hz, 2H), 3.78-3.67 (m, 2H), 3.51-3.37 (m, 2H), 2.77-2.73 (m. 3H), 2.13-2.05 (m, 2H), 1.42 (s, 9H), 1.33 (t, J=7.0 Hz, 3H).

### Preparation of 2-((1-(tert-butoxycarbonyl) piperidin-4-yl)amino)-4-ethoxypyrimidine-5-carboxylic acid

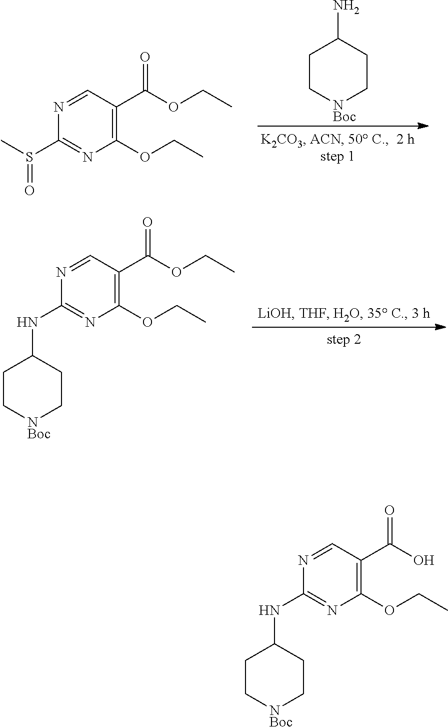 [SMILES: CCOC(=O)c1cnc(S(C)=O)nc1OCC ; CC(C)(C)OC(=O)N1CCC(N)CC1 ; CCOC(=O)c1cnc(NC2CCN(C(=O)OC(C)(C)C)CC2)nc1OCC ; CCOc1nc(NC2CCN(C(=O)OC(C)(C)C)CC2)ncc1C(=O)O]

### Step 1: Preparation of ethyl 2-((1-(tert-butoxycarbonyl) piperidin-4-yl)amino)-4-ethoxypyrimidine-5-carboxylate

To a solution of ethyl 4-ethoxy-2-(methylsulfinyl)pyrimidine-5-carboxylate (5 g, 19.38 mmol) in CH CN (60 mL) were added K₂CO₃ (5.34 g, 38.76 mmol) and tert-butyl 4-aminopiperidine-1-carboxylate (11.6 g, 58.14 mmol), the mixture was stirred at 50° C. for 2 h. The reaction mixture was concentrated in vacuo to remove CH₃CN. The crude was diluted with water (30 mL) and extracted with EA (30 mL×3). The organic layer was washed with brine, dried over Na₂SO₄ and concentrated in vacuo. The crude was purified with column chromatography (PE:EA=5:1) to give title compound (3 g, 39.4%) as a white solid. ESI-MS (M+H)⁺: 395.2.

### Step 2: Preparation of 2-((1-(tert-butoxycarbonyl) piperidin-4-yl)amino)-4-ethoxypyrimidine-5-carboxylic acid

To a mixture of ethyl 2-((1-(tert-butoxycarbonyl) piperidin-4-yl)amino)-4-ethoxypyrimidine-S-carboxylate (3 g, 7.60 mmol) in THF: H₂O (10 mL: 10 mL) was added LiOH (0.64 g, 15.20 mmol), the mixture was stirred at 35° C. for 3 h. The reaction mixture was concentrated in vacuo to remove most THF. The mixture was adjusted to pH=5 with 1M HCl. The precipitate was filtered to give title product (2 g. 72%) as a white solid. ESI-MS (M+H)⁺: 367.2.

### Preparation of 2-((1-(tert-butoxycarbonyl) piperidin-4-yl) (methyl) amino)-4-methoxypyrimidine-5-carboxylic acid

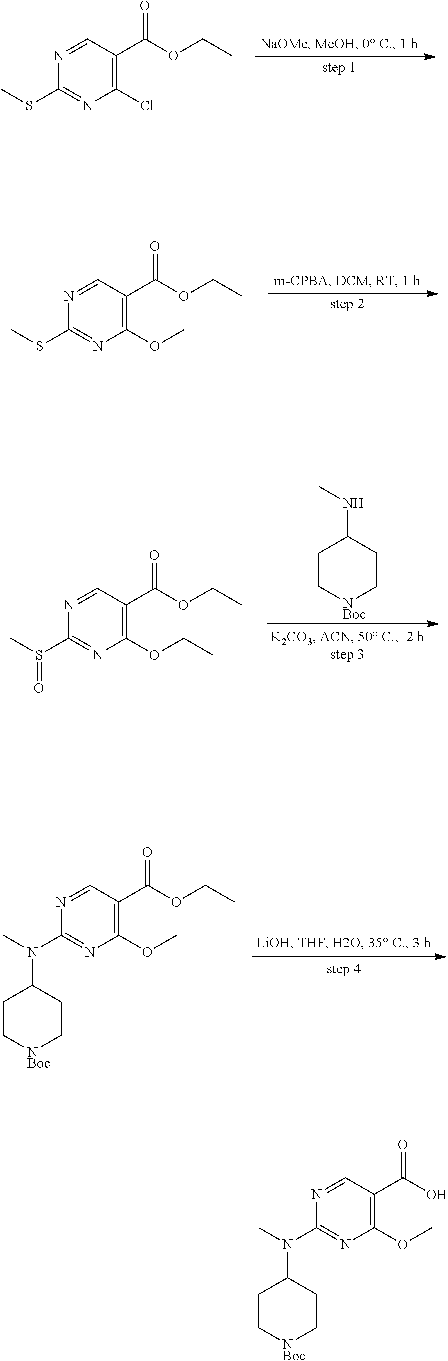

### Step 1: Preparation of ethyl 4-methoxy-2-(methylthio)pyrimidine-5-carboxylate

To a solution of ethyl 4-chloro-2-(methylthio)pyrimidine-S-carboxylate (10 g, 0.043 mol) in MeOH (100 mL) was added NaOMe (2.56 g, 0.047 mol). The mixture was stirred at 0° C. for 1 h. The mixture was concentrated in vacuo to remove most MeOH. The crude was diluted with water (80 mL) and exacted with EA (100 mL×3). The organic layer was washed with brine, dried over Na₂SO₄ and concentrated in vacuo to give title product (9.5 g, 0.97%) as a yellow solid. ESI-MS (M+H)⁺: 229.1

### Step 2: Preparation of ethyl 4-methoxy-2-(methylsulfinyl)pyrimidine-5-carboxylate

To a solution of ethyl 4-methoxy-2-(methylthio)pyrimidine-5-carboxylate (9.5 g, 0.042 mol) in DCM (100 mL) was added m-CPBA (10.75 g, 0.060 mol). The mixture was stirred at rt for 1 h. The mixture was diluted with water (100 mL). The organic layer was washed with bine, dried over Na₂SO₄ and concentrated in vacuo to give crude title product (9 g, 90%) as a white solid. ESI-MS (M+H)⁺: 245.1.

### Step 3: Preparation of ethyl 2-((1-(tert-butoxycarbonyl) piperidin-4-yl) (methyl) amino)-4-methoxypyrimidine-5-carboxylate

To a solution of ethyl 4-methoxy-2-(methylsulfinyl)pyrimidine-5-carboxylate (2 g, 8.20 mmol) in CH—CN (30 mL) were added K₂CO₃ (2.3 g, 16.40 mmol) and tert-butyl 4-methylpiperazine-1-carboxylate (2.6 g, 12.30 mol), the mixture was stirred at 50° C. for 1 h. The reaction mixture was concentrated in vacuo to remove CH₃CN. The crude was diluted with water (30 mL) and extracted with EA (30 mL×3). The organic layer was washed with brine, dried over Na₂SO₄ and concentrated in vacuo. The crude was purified with column chromatography (PE:EA=5:1) to give title compound (2.5 g, 78%) as a white solid. ESI-MS (M+H)⁺: 395.3.

### Step 4: Preparation of 2-((1-(tert-butoxycarbonyl) piperidin-4-yl) (methyl) amino)-4-methoxypyrimidine-5-carboxylic acid

To a mixture of ethyl 2-((1-(tert-butoxycarbonyl) piperidin-4-yl) (methyl) amino)-4-methoxypyrimidine-5-carboxylate (2.5 g, 6.30 mmol) in THF: H₂O (10 mL: 10 mL) was added LiOH (0.8 g, 18.90 mmol), the mixture was stirred at 35° C. for 3 h. The reaction mixture was concentrated in vacuo to remove most THF. The mixture was adjusted to pH=5 with 1M HCl. The precipitate was filtered and dried under vacuum to give title product (1 g, 43.4%) as a white solid. ESI-MS (M+H)⁺: 367.2.

### Preparation of 2-((1-(tert-butoxycarbonyl) piperidin-4-yl) (methyl) amino)-4-methoxypyrimidine-5-carboxylic acid

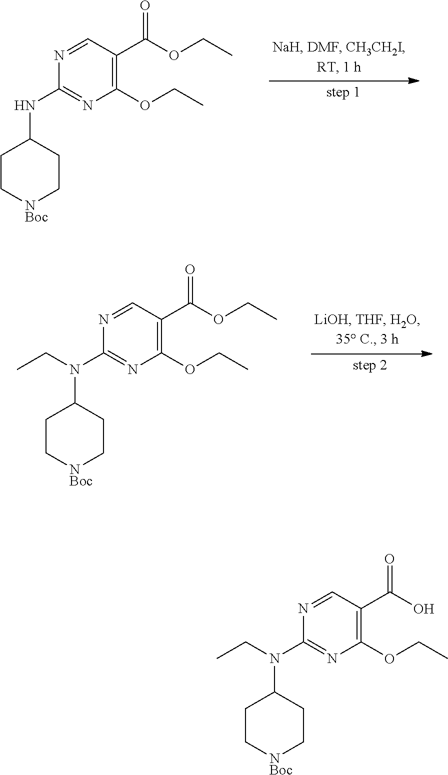 [SMILES: CCOC(=O)c1cnc(NC2CCN(C(=O)OC(C)(C)C)CC2)nc1OCC ; CCOC(=O)c1cnc(N(CC)C2CCN(C(=O)OC(C)(C)C)CC2)nc1OCC ; CCOc1nc(N(CC)C2CCN(C(=O)OC(C)(C)C)CC2)ncc1C(=O)O]

### Step 1: Preparation of ethyl 2-((1-(tert-butoxycarbonyl) piperidin-4-yl) (ethyl) amino)-4-ethoxypyrimidine-5-carboxylate

To a mixture of ethyl 2-((1-(tert-butoxycarbonyl) piperidin-4-yl)amino)-4-ethoxypyrimidine-5-carboxylate (3 g. 7.60 mmol) in DMF (20 mL) were added NaH (0.64 g. 15.20 mmol) and CH₃CH₂I (20 mL). The mixture was stirred at rt for 1 h. The mixture was diluted with water (50 mL) and extracted with EA (80 mL×3). The organic layer was washed with brine, dried over Na₂SO₄ and concentrated in vacuo. The crude was purified with column chromatography (PE:EA=5:1) to give title compound (1.1 g, 34%) as a white solid. ESI-MS (M+H)⁺: 423.3.

### Step 2: Preparation of 2-((1-(tert-butoxycarbonyl) piperidin-4-yl) (ethyl) amino)-4-ethoxypyrimidine-5-carboxylic acid

To a mixture of ethyl 2-((1-(tert-butoxycarbonyl) piperidin-4-yl) (ethyl) amino)-4-ethoxypyrimidine-5-carboxylate (1.1 g, 2.60 mmol) in THF: H₂O (10 mL: 10 mL) was added LiOH (0.22 g, 5.20 mmol), the mixture was stirred at 35° C. for 3 h. The reaction mixture was concentrated in vacuo to remove most THF. The mixture was adjusted to pH=5 with 1M HCl. The precipitate was filtered and dried under vacuum to give title product (450 mg, 44%) as a white solid. ESI-MS (M+H)⁺: 395.2.

### Preparation of Starting Material: 8-fluoro-2-methylimidazo[1,2-a]pyridin-6-amine

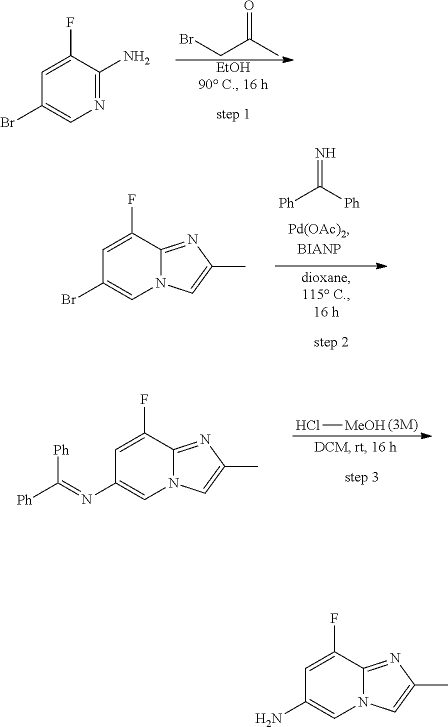

### Step 1:6-bromo-8-fluoro-2-methylimidazo[1,2-a]pyridine

A mixture of 5-bromo-3-fluoropyridin-2-amine (50 g, 263 mmol) and 1-bromopropan-2-one (89.5 g, 658 mmol) in EtOH (500 mL) was heated to 90° C. for 16 h. The reaction was cooled to r.t, the precipitated solids were collected by filtration and washed with EtOH. The cake was dissolved in H₂O and basified to pH 10 with Na₂CO₃, the precipitated solids were collected by filtration, washed with H2O and dried by vacuum to afford 6-bromo-8-fluoro-2-methylimidazo[1,2-a]pyridine (40 g. Y: 67%) as a white solid. ESI-MS (M+H)⁺: 229.1, 231.1. H NMR (400 MHZ, DMSO-d₆) δ 8.71 (d, J=1.4 Hz, 1H), 7.82-7.73 (m, 1H), 7.38 (dd, J=10.7, 1.5 Hz, 1H), 2.35 (d, J=0.6 Hz, 3H).

### Step 2: N-(8-fluoro-2-methylimidazo[1,2-a]pyridin-6-yl)-1,1-diphenylmethanimine

A mixture of 6-bromo-8-fluoro-2-methylimidazo[1,2-a]pyridine (15 g, 65.8 mmol), diphenylmethanimine (12.5 g, 69.1 mmol), Pd(OAc)₂ (1.48 g, 6.58 mmol), BINAP (8.2 g, 13.2 mmol) and Cs₂CO₃ (42.8 g, 131.6 mmol) in dioxane (150 mL) was purged with N₂ for three times at rt. Then the mixture was heated to 115° C. for 16 h. The mixture was cooled to r.t and filtered and filter was concentrated under reduce pressure. The residue was purified by silica gel column (0-50% EA in PE) to provide N-(8-fluoro-2-methylimidazo[1,2-a]pyridin-6-yl)-1,1-diphenylmethanimine (16 g, yield: 74%) as light brown solid. ESI-MS (M+H)⁺: 330.4. ¹H NMR (400 MHZ, DMSO-d₆) δ 7.83 (d, J=1.2 Hz, 1H), 7.68 (d, J=7.3 Hz, 2H), 7.62 (d, J=2.8 Hz, 1H), 7.56 (d, J=7.2 Hz, 1H), 7.48 (t, J=7.5 Hz, 2H), 7.43-7.35 (m, 3H), 7.24-7.21 (m, 2H), 6.65-6.63 (m, 1H), 2.27 (s, 3H).

### Step 3:8-fluoro-2-methylimidazo[ 1,2-a]pyridin-6-amine

To a solution of N-(8-fluoro-2-methylimidazo[1,2-a]pyridin-6-yl)-1,1-diphenylmethanimine (15 g, 45.6 mmol) in DCM (100 mL) at 0° C. was added HCl-dioxane (4M, 22.8 mL, 91.2 mmol). The mixture was stirred for 16 hours at rt. After concentration, the residue was treated with EA. The residue was dissolved in H₂O and basified to pH 10 with Na₂CO₃, the precipitated solids were collected by filtration, washed with H₂O and dried by vacuum to afford 8-fluoro-2-methylimidazo[1,2-a]pyridin-6-amine (7.2 g, yield: 96%) as yellow solid. ESI-MS (M+H): 166.1. ¹H NMR (400 MHZ, DMSO-d₆) δ 8.04 (s, 1H), 7.85 (s, 1H), 7.38 (d, J=12.3 Hz, 1H), 3.57 (s, 2H), 2.43 (s, 3H).

### Preparation of Starting Material: (R)-2-(3-((tert-butoxycarbonyl) (methyl) amino) pyrrolidin-1-yl)-4-ethoxypyrimidine-5-carboxylic acid

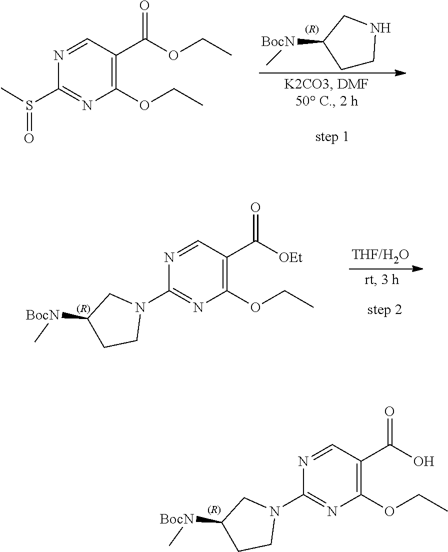

### Step 1: ethyl(R)-2-(3-((tert-butoxycarbonyl) (methyl) amino) pyrrolidin-1-yl)-4-ethoxypyrimidine-5-carboxylate

To a stirred solution of ethyl 4-ethoxy-2-(methylsulfinyl)pyrimidine-5-carboxylate (20 g, 87 mmol) in MeCN (200 mL) was added tert-butyl(R)-methyl(pyrrolidin-3-yl) carbamate (17.4 g, 87 mmol) and potassium carbonate (23.6 g, 174 mmol). The mixture was stirred for 2 hours at 50° C. After concentration, the residue was treated with EA/Water (300 mL, 1:1). The organic phase was separated and the aqueous phase was extracted with EA (150 mL×3). The combined organics was dried over sodium sulfate. After concentration under reduced pressure, crude product ethyl(R)-2-(3-((tert-butoxycarbonyl) (methyl) amino) pyrrolidin-1-yl)-4-ethoxypyrimidine-5-carboxylate (28 g, Y: 92%) as a white solid, which was used to next step without further purification. ESI-MS (M+H)⁺: 395.5. ¹H NMR (400 MHz, CDCl₃) δ 8.74 (s, 1H), 4.86 (br.s, 1H), 4.48 (q. J=7.1 Hz, 2H), 4.29 (q, J=7.1 Hz, 2H), 3.85-3.82 (m, 2H), 3.59-3.41 (m, 2H), 2.82 (s, 3H), 2.23-2.07 (m, 2H), 1.48 (s, 9H), 1.43 (t, J=7.1 Hz, 3H), 1.34 (t, J=7.1 Hz, 3H).

### Step 2: (R)-2-(3-((tert-butoxycarbonyl) (methyl) amino) pyrrolidin-1-yl)-4-ethoxypyrimidine-5-carboxylic acid

To a solution of ethyl(R)-2-(3-((tert-butoxycarbonyl) (methyl) amino) pyrrolidin-1-yl)-4-ethoxypyrimidine-5-carboxylate (28 g, 71.0 mmol) in THF (280 mL) and H₂O (28 ml) was added LiOH.H₂O (7.5 g, 177.5 mmol). The reaction mixture was stirred for 3 hours at rt. After concentration, the residue was diluted with water (100 mL) and adjusted pH to 5˜6 with 1 N HCl aqueous solution. The solid was formed and collected by filtration, washed with water for three times (50 mL×3). The solid was dried under vacuum at 55° C. to provide title product (16 g, yield: 86%) as yellow solid. ESI-MS (M+H)⁺: 367.5. ¹H NMR (400 MHZ, DMSO-d₆)δ 12.16 (s, 1H), 8.60 (s, 1H), 4.67 (br.s, 1H), 4.42 (q, J=7.0 Hz, 2H), 3.83-3.68 (m, 2H), 3.53-3.37 (m, 2H), 2.75 (s, 3H), 2.13-2.04 (m, 2H), 1.42 (s, 9H), 1.32 (t, J=7.0 Hz, 3H).

### Preparation of Starting Material: 4-ethoxy-N-(8-fluoro-2-methylimidazo[1,2-a]pyridin-6-yl)-2-(methylsulfinyl)pyrimidine-5-carboxamide

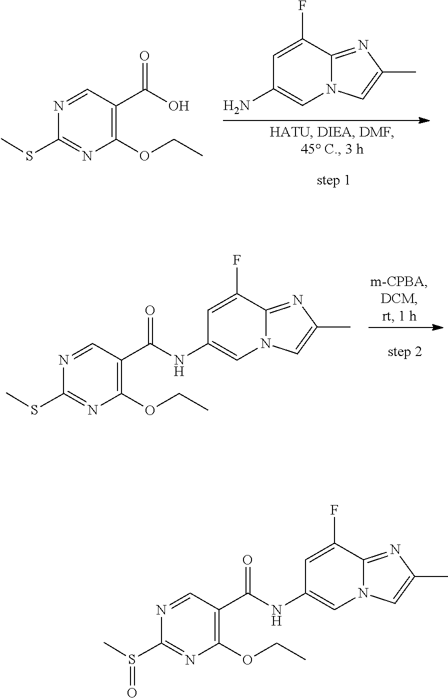

### Step 1: Preparation of 4-ethoxy-N-(8-fluoro-2-methylimidazo[1,2-a]pyridin-6-yl)-2-(methylthio)pyrimidine-5-carboxamide

To a solution of 4-ethoxy-2-(methylthio)pyrimidine-5-carboxylic acid (3.5 g, 14.52 mmol) in DMF (40 mL) were added DIEA (8.44 g, 58.09 mmol), HATU (8.14 g, 21.38 mmol) and 8-fluoro-2-methylimidazo[1,2-a]pyridin-6-amine (3.2 g, 17.42 mmol). The reaction mixture was stirred at 45° C. for 3 h. The mixture was diluted with water (200 mL). The precipitate was filtered, washed with water and dried in vacuo to give title product (3 g, 59.3%) as a black solid. ESI-MS (M+H)⁺: 362.0.

### Step 2: Preparation of 4-ethoxy-N-(8-fluoro-2-methylimidazo[1,2-a]pyridin-6-yl)-2-(methylsulfinyl)pyrimidine-5-car boxamide

To a solution of 4-ethoxy-N-(8-fluoro-2-methylimidazo[1,2-a]pyridin-6-yl)-2-(methylthio)pyrimidine-5-carboxamide (1.00 g, 2.77 mmol) in DCM (10 mL) was added m-CPBA (0.71 g, 4.16 mmol). The mixture was stirred at rt for 1 h. The mixture was extracted with water (30 mL×3). The organic layer was washed with brine, dried over Na₂SO₄ and concentrated in vacuo to give title product (1.0 g, 95.8%) as a white solid. ESI-MS (M+H)⁺: 378.4.

### Example 30. (S)-2-(3-((dimethylamino) methyl) piperidin-1-yl)-4-ethoxy-N-(8-fluoro-2-methylimidazo[1,2-a]pyridin-6-yl)pyrimidine-5-carboxamide formate (Compound 250)

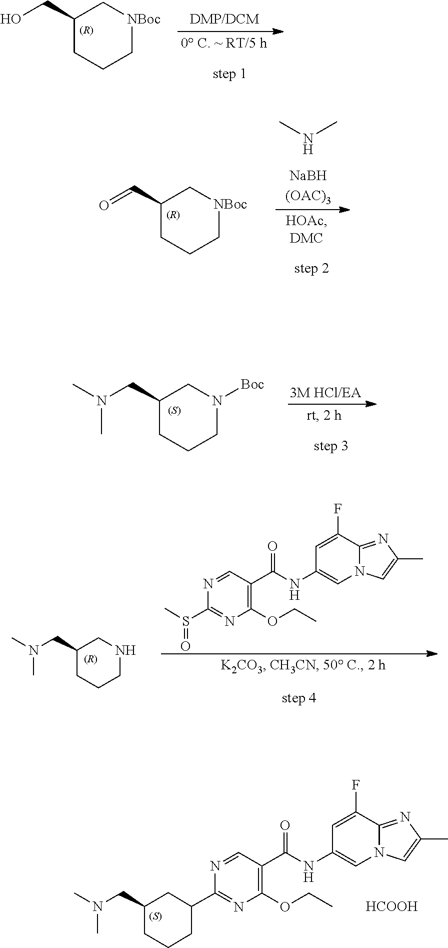

### Step 1: Preparation of tert-butyl(R)-3-formylpiperidine-1-carboxylate

To a mixture of tert-butyl(R)-3-(hydroxymethyl) piperidine-1-carboxylate (4.0 g. 18.60 mmol) in DCM (20 mL) was added Dess-Martin reagent (500 mg, 2.90 mmol) at 0° C. The mixture was stirred at rt for 5 h. The mixture was diluted with water (50 mL), extracted with DCM (100 mL) The organic layer was washed with brine, dried with Na₂SO₄ and concentration in vacuo. The crude was purified by silica gel column chromatography (PE: EA=5:1) to give title product (2.8 g, 71.8%) as a yellow oil. ¹H NMR (400 MHZ, CDCl₃)δ 9.70 (s, 1H), 3.93-3.92 (m, 1H), 3.70-3.61 (m, 1H), 3.34-3.32 (m, 1H), 3.10-3.09 (m, 1H), 2.44-2.43 (m, 1H), 1.96 (d, J=6.7 Hz, 1H), 1.68-1.66 (m, 2H), 1.52-1.51 (m, 1H), 1.46 (s, 9H).

### Step 2: Preparation of tert-butyl(S)-3-((dimethylamino) methyl) piperidine-1-carboxylate

To a mixture of tert-butyl(R)-3-formylpiperidine-1-carboxylate (1.0 g, 4.70 mmol) in DCM (10 mL) were added dimethylamine (0.21 mL, 0.42 mmol, 2M in THF) and HOAc (115 mg, 1.4 mmol). The mixture was stirred at 50° C. for 1 h. Then NaBH(OAc)₃ (36 mg, 0.571 mmol) was added and the mixture was stirred at 50° C. for 2 h. The reaction mixture was diluted with water (50 mL), extracted with EA (50 mL×3). The organic layer was washed with brine, dried with Na₂SO₄ and concentration in vacuo. The crude was purified by silica gel column chromatography (PE/EA=5:1) to afford title product (400 mg. Y: 36.4%) as a yellow oil. ¹H NMR (400 MHZ, CDCl₃)δ 4.09-3.75 (m, 2H), 3.48-3.48 (m, 2H), 2.84-2.54 (m, 2H), 2.46 (s, 6H), 1.98-1.84 (m, 1H), 1.84-1.73 (m, 1H), 1.70-1.62 (m, 1H), 1.57-1.34 (m, 10H), 1.27-1.19 (m, 1H).

### Step 3: Preparation of (R)—N,N-dimethyl-1-(piperidin-3-yl) methanamine hydrochloride

To a solution of tert-butyl(S)-3-((dimethylamino) methyl) piperidine-1-carboxylate (400 mg, 1.65 mmol) in EA (5 mL) was added 3M HCl/EA (10 mL). The mixture was stirred at rt for 2 h. After concentration, the residue was diluted with water (20 mL), exacted with EA (20 mL×3). The aqueous phase was concentrated in vacuo and lyophilized to give title product (250 mg, yield: 85.4%) as a yellow solid. ESI-MS (M+H)⁺: 143.1.

### Step 4: Preparation of(S)-2-(3-((dimethylamino) methyl) piperidin-1-yl)-4-ethoxy-N-(8-fluoro-2-methylimidazo[1,2-a]pyridin-6-yl)pyrimidine-5-carboxamide formate

To a mixture of (R)—N,N-dimethyl-1-(piperidin-3-yl) methanamine hydrochloride (250 mg, 1.40 mmol) in CH₃CN (3 mL) were added 4-ethoxy-N-(8-fluoro-2-methylimidazo[1,2-a]pyridin-6-yl)-2-(methylsulfinyl)pyrimidine-5-carboxamide (80 mg, 0.39 mmol) and K₂CO₃ (73 mg, 0.53 mmol). The mixture was stirred at 50° C. for 2 h. The mixture was diluted H₂O (5 mL), extracted with EA (10 mL×3). The organic layer was concentrated in vacuo. The crude was purified by prep-HPLC (0.05% FA in water/ACN) to give title product (18 mg. Y: 2.04%) as a yellow solid. ESI-MS (M+H)⁺: 456.3. ¹H NMR (400 MHZ, DMSO-d₆) δ 9.56 (s, 1H), 9.07 (d, J=1.4 Hz, 1H), 8.62 (s, 1H), 8.21 (s, 1H), 7.88 (d, J=2.9 Hz, 1H), 7.21 (dd, J=12.6, 1.4 Hz, 1H), 4.79-4.60 (m, 1H), 4.57-4.48 (m, 3H), 3.10-3.02 (m, 1H), 2.82-2.68 (m, 1H), 2.35-2.32 (m, 3H), 2.23-2.17 (m, 1H), 2.16-2.13 (m, 6H), 2.07-2.02 (m, 1H), 1.81-1.75 (m, 1H), 1.73-1.62 (m, 2H), 1.47-1.39 (m, 4H), 1.27-1.18 (m, 1H).

### Example 31. N-(2,8-dimethylimidazo[1,2-a]pyrazin-6-yl)-4-ethoxy-2-(piperidin-4-ylamino) pyrimidine-5-carboxamide formate (Compound 251)

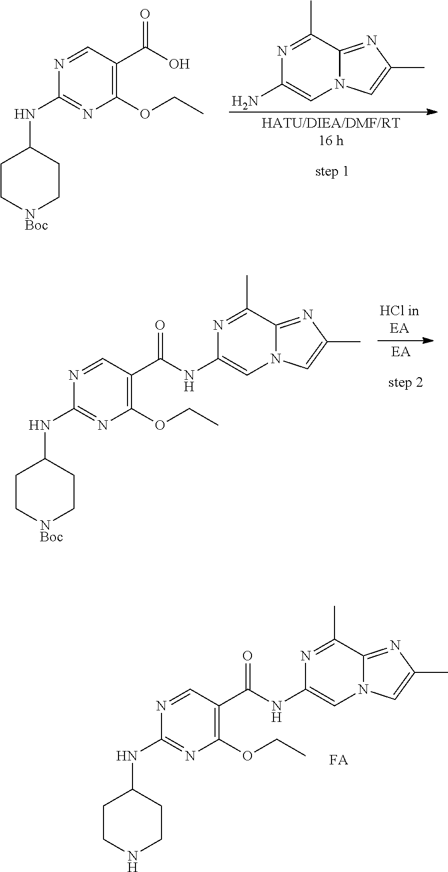

### Step 1: Preparation of tert-butyl 4-((5-((2,8-dimethylimidazo[1,2-a]pyrazin-6-yl) carbamoyl)-4-ethoxypyrimidin-2-yl) amino) piperidine-1-carboxylate

To a mixture of 2-((1-(tert-butoxycarbonyl) piperidin-4-yl)amino)-4-ethoxypyrimidine-5-carboxylic acid (150 mg, 0.41 mmol) in DMF (5 mL) were added 2,8-dimethylimidazo[1,2-a]pyrazin-6-amine (67 mg, 0.41 mmol), HATU (234 mg, 0.61 mmol) and DIEA (159 mg, 1.23 mmol). The mixture was stirred at rt for 16 h. The mixture was diluted with H₂O (5 mL) and the precipitate was filtered and dried in vacuo to give crude title product (150 mg, Y: 71.8%) as a yellow solid. ESI-MS (M+H)⁺: 511.2.

### Step 2: Preparation of N-(2,8-dimethylimidazo[1,2-a]pyrazin-6-yl)-4-ethoxy-2-(piperidin-4-ylamino) pyrimidine-5-carboxamide formate

A mixture of tert-butyl 4-((5-((2,8-dimethylimidazo[1,2-a]pyrazin-6-yl) carbamoyl)-4-ethoxypyrimidin-2-yl) amino) piperidine-1-carboxylate (150 mg, 0.29 mmol) in 3 M HCl/EA (5 mL) was stirred at rt for 2 h. The mixture was diluted with H₂O (5 mL), extracted with EA (10 mL×3). The aqueous layer was concentrated in vacuo and the residue was purified by prep-HPLC (0.05% FA in water/ACN) to give title product (73 mg, 54.4%) as a yellow solid. ESI-MS (M+H⁺): 411.1. 1 H NMR (400 MHZ, DMSO-d₆) δ 9.73 (d, J=11.9 Hz, 1H), 9.14 (s, 1H), 8.71 (d, J=7.6 Hz, 1H), 8.34 (s, 1H), 8.19-7.97 (m, 1H), 7.96 (s. 1H), 4.62-4.51 (m, 2H), 4.09-3.92 (m, 1H), 3.23-3.14 (m, 2H), 2.92-2.78 (m, 2H), 2.67 (s, 3H), 2.37 (s, 3H), 2.02-1.91 (m, 2H), 1.68-1.56 (m, 2H), 1.48 (s, 3H).

### Example 32. 4-ethoxy-2-(4-ethylpiperazin-1-yl)-N-(7-fluoro-2-methyl-2H-indazol-5-yl)pyrimidine-5-carboxamide hydrochloride (Compound 252)

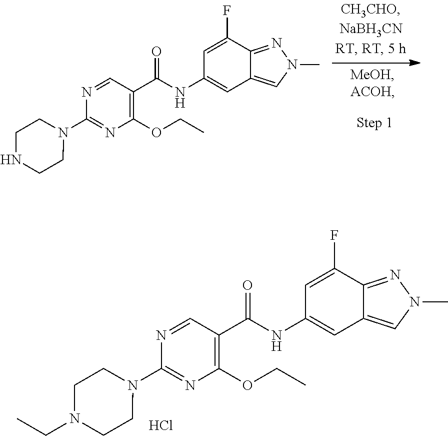

### Step 1: Preparation of 4-ethoxy-2-(4-ethylpiperazin-1-yl)-N-(7-fluoro-2-methyl-2H-indazol-5-yl)pyrimidine-5-carboxamide hydrochloride

To a mixture of 4-ethoxy-N-(7-fluoro-2-methyl-2H-indazol-5-yl)-2-(piperazin-1-yl)pyrimidine-5-carboxamide (80 mg, 0.2 mmol) and AcOH (36 mg, 0.6 mmol) in MeOH (10 mL) were added NaBH₃CN (63 mg, 1 mmol) and CH₃CHO (44 mg, 1 mmoL). The mixture was stirred at rt for 5 h. LCMS showed the starting material was consumed completely. Filtered and the filtrate was concentrated in vacuo. The residue was purified by prep-HPLC (0.05% HCl in water/ACN) to give title product (32.5 mg, Y: 35.1%) as a yellow solid. ESI-MS (M+H⁺): 397.1. ¹H NMR (400 MHZ, DMSO-d₆) δ 11.01 (s, 1H), 9.71 (s, 1H), 8.68 (s, 1H), 8.42 (d, J=2.8 Hz, 1H), 7.98 (s, 1H), 7.34-7.27 (m, 1H), 4.81-4.74 (m, 2H), 4.54-4.52 (m, 2H), 4.18 (s, 3H), 3.59-3.46 (m, 4H), 3.19-3.11 (m, 2H), 3.08-2.97 (m, 2H), 1.44 (t, J=7.0 Hz, 3H), 1.28 (t, J=7.3 Hz, 3H).

### Example 33. N-(2,8-dimethylimidazo[1,2-a]pyrazin-6-yl)-4-methoxy-2-(methyl(piperidin-4-yl) amino) pyrimidine-5-carboxamide formate (Compound 253)

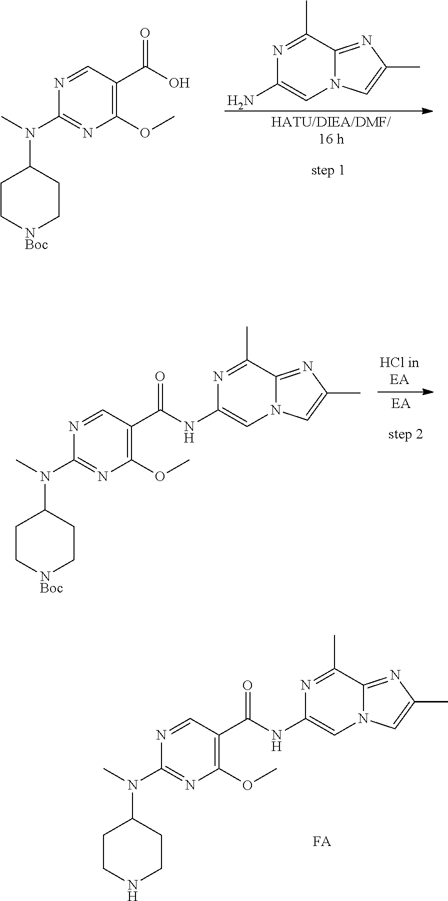 [SMILES: COc1nc(N(C)C2CCN(C(=O)OC(C)(C)C)CC2)ncc1C(=O)O ; Cc1cn2cc(N)nc(C)c2n1 ; COc1nc(N(C)C2CCN(C(=O)OC(C)(C)C)CC2)ncc1C(=O)Nc1cn2cc(C)nc2c(C)n1 ; COc1nc(N(C)C2CCNCC2)ncc1C(=O)Nc1cn2cc(C)nc2c(C)n1 ; "FA"]

### Step 1: Preparation of tert-butyl 4-((5-((2,8-dimethylimidazo[1,2-a]pyrazin-6-yl) carbamoyl)-4-methoxypyrimidin-2-yl) (methyl) amino) piperidine-1-carboxylate

To a mixture of 2-((1-(tert-butoxycarbonyl) piperidin-4-yl) (methyl) amino)-4-methoxypyrimidine-5-carboxylic acid (150 mg, 0.41 mmol) in DMF (5 mL) were added 2,8-dimethylimidazo[1,2-a]pyrazin-6-amine (67 mg, 0.41 mmol), HATU (234 mg, 0.61 mmol) and DIEA (159 mg, 1.23 mmol). The mixture was stirred at rt for 16 h. The mixture was diluted with H₂O (5 mL) and the precipitate was filtered and dried in vacuo to give crude title product (150 mg, Y: 71.8%) as a yellow solid. ESI-MS (M+H)⁺: 511.2. ¹H NMR (400 MHZ, DMSO-d₆) δ 9.61 (s, 1H), 9.16 (s, 1H), 8.74 (s, 1H), 7.95 (s, 1H), 4.92-4.56 (m, 1H), 4.11 (s, 3H), 4.08-4.02 (m, 1H), 3.28-3.24 (m, 1H), 3.07 (s, 3H), 2.93-2.77 (m, 2H), 2.69 (s, 3H), 2.38 (s, 3H), 1.72-1.60 (m, 4H), 1.42 (s, 9H).

### Step 2: Preparation of N-(2,8-dimethylimidazo[1,2-a]pyrazin-6-yl)-4-methoxy-2-(methyl(piperidin-4-yl) amino) pyrimidine-5-carboxamide formate

A mixture of tert-butyl 4-((5-((2,8-dimethylimidazo[1,2-a]pyrazin-6-yl) carbamoyl)-4-methoxypyrimidin-2-yl) (methyl) amino) piperidine-1-carboxylate (150 mg, 0.29 mmol) in 3 M HCl/EA (S mL) was stirred at rt for 2 h. The mixture was diluted with H₂O (5 mL), extracted with EA (10 mL×3). The aqueous layer was concentrated in vacuo and the residue was purified by prep-HPLC (0.05% FA in water/ACN) to give title product (40 mg, 29.8%) as a yellow solid. ESI-MS (M+H⁺): 411.2. 1 H NMR (400 MHZ, DMSO-d₆) δ 9.61 (s, 1H), 9.16 (s, 1H), 8.74 (s, 1H), 8.32 (s, 1H), 7.95 (s, 1H), 4.89-4.62 (m, 1H), 4.11 (s, 3H), 3.18 (d, J=11.2 Hz, 2H), 3.08 (s, 3H), 2.77 (t, J=11.8 Hz, 2H), 2.69 (s, 3H), 2.38 (s, 3H), 1.86-1.67 (m, 4H).

### Example 34. 4-ethoxy-N-(7-fluoro-2-methyl-2H-indazol-5-yl)-2-(3-(methylamino) azetidin-1-yl)pyrimidine-5-carboxamide hydrochloride (Compound 254)

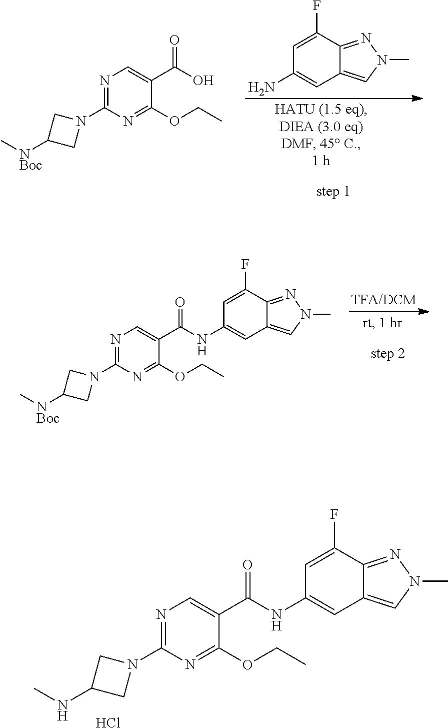

### Step 1: synthesis of tert-butyl(1-(4-ethoxy-5-((7-fluoro-2-methyl-2H-indazol-5-yl) carbamoyl)pyrimidin-2-yl) azetidin-3-yl) (methyl) carbamate

A solution of 2-(3-((tert-butoxycarbonyl) (methyl) amino) azetidin-1-yl)-4-ethoxypyrimidine-5-carboxylic acid (200 mg, 0.57 mmol), 7-fluoro-2-methyl-2H-indazol-5-amine (94 mg, 0.57 mmol), HATU (324 mg, 0.85 mmol) and DIEA (293 mg, 2.27 mmol) in DMF (5 mL) was stirred for 1 h at 45° C. The reaction mixture was purified by reverse phase column (ACN in water, 70%, 1% OFA) to give title product (130 mg, Y: 46%) as a white solid. ESI-MS (M+H)⁺: 500.3.

### Step 2: synthesis of 4-ethoxy-N-(7-fluoro-2-methyl-2H-indazol-5-yl)-2-(3-(methylamino) azetidin-1-yl)pyrimidine-5-carboxamide hydrochloride

A solution of tert-butyl(1-(4-ethoxy-5-((7-fluoro-2-methyl-2H-indazol-5-yl) carbamoyl)pyrimidin-2-yl) azetidin-3-yl) (methyl) carbamate (130 mg, 0.26 mmol) in DCM (3 mL) and TFA (1 mL) was stirred at rt for 1 h. After concentration, the residue was purified by pre-HPLC (MeCN/0.05% HCl in water, 35%) to give title product (70 mg, Y: 62%) as a white solid. ESI-MS (M+H)⁺: 400.2. ¹H NMR (400 MHZ, MeOD-d₄)δ 8.68 (s, 1H), 8.30 (d, J=2.5 Hz, 1H), 7.93 (s, 1H), 7.27 (d, J=11.9 Hz, 1H), 4.75-4.70 (m, 4H), 4.55 (dd, J=11.1, 4.0 Hz, 2H), 4.37-4.30 (m, 1H), 4.23 (s, 3H), 2.80 (s, 3H), 1.56 (t, J=7.1 Hz, 3H).

### Example 35. 4-ethoxy-N-(2-methyl-2H-indazol-5-yl)-2-(3-(methylamino) azetidin-1-yl)pyrimidine-5-carboxamide (Compound 255)

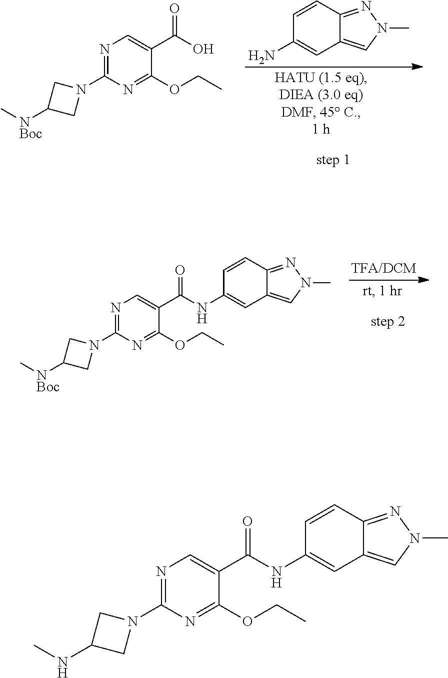

### Step 1: synthesis of tert-butyl(1-(4-ethoxy-5-((2-methyl-2H-indazol-5-yl) carbamoyl)pyrimidin-2-yl) azetidin-3-yl) (methyl) carbamate

A solution of 2-(3-((tert-butoxycarbonyl) (methyl) amino) azetidin-1-yl)-4-ethoxypyrimidine-5-carboxylic acid (200 mg, 0.57 mmol), 2-methyl-2H-indazol-5-amine (84 mg, 0.57 mmol), HATU (324 mg. 0.85 mmol) and DIEA (293 mg, 2.27 mmol) in DMF (5 mL) was stirred for 1 h at 45° C. After concentration, the residue was purified by reverse phase column (0.1% FA in water/CH₃CN) to give title product (100 mg, Y: 37%) as a white solid. ESI-MS (M+H)⁺: 482.3. ¹H NMR (400 MHZ, DMSO-d₆) δ 9.59 (s, 1H), 8.63 (s, 1H), 8.28 (s, 1H), 8.23 (s, 1H), 7.57 (d, J=9.1 Hz, 1H), 7.27 (d, J=9.2 Hz, 1H), 4.87 (br s, 1H), 4.52 (q, J=7.0 Hz, 2H), 4.30 (t, J=8.9 Hz, 2H), 4.18 (dd, J=9.8, 6.0 Hz, 2H), 4.14 (s, 3H), 2.88 (s, 3H), 1.44 (t, J=7.1 Hz, 3H), 1.41 (s, 9H).

### Step 2: synthesis of 4-ethoxy-N-(2-methyl-2H-indazol-5-yl)-2-(3-(methylamino) azetidin-1-yl)pyrimidine-5-carboxamide

A solution of tert-butyl(1-(4-ethoxy-5-((2-methyl-2H-indazol-5-yl) carbamoyl)pyrimidin-2-yl) azetidin-3-yl) (methyl) carbamate (130 mg, 0.26 mmol) in DCM (3 mL) and TFA (1 mL) was stirred at rt for 1 h. After concentration, the residue was purified by pre-HPLC (MeCN in water, 45%, 0.1% NH₄OH) to give title product (60 mg, Y: 76%) as a white solid. ESI-MS (M+H)⁺: 382.2. ¹H NMR (400 MHz, DMSO d₆) δ 9.56 (s, 1H), 8.61 (s, 1H), 8.27-8.24 (m, 2H), 7.55 (s, 1H), 7.25 (s, 1H), 4.50 (br s, 2H), 4.17-4.14 (m, 5H), 3.80 (br s, 2H), 3.59 (br s, 1H), 2.26 (s, 3H), 1.45 (s, 3H).

### Example 36. 4-ethoxy-2-(methyl(piperidin-4-yl) amino)-N-(2-methylimidazo[1,2-a]pyrazin-6-yl)pyrimidine-5-carboxamide (Compound 256)

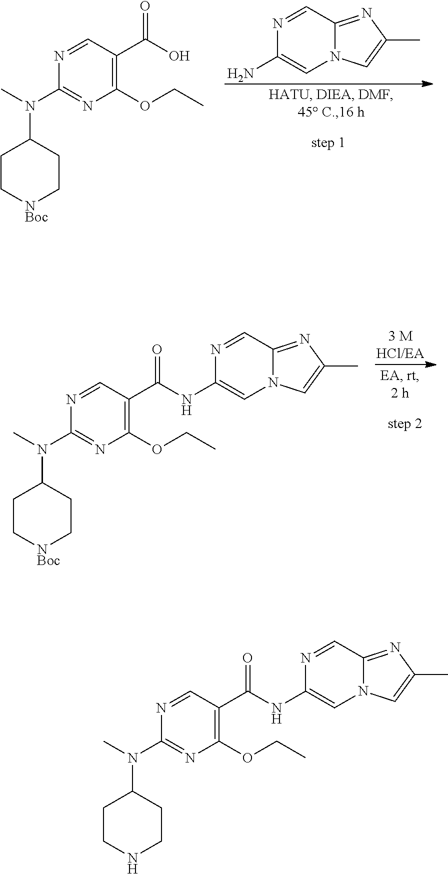 [SMILES: CCOc1nc(N(C)C2CCN(C(=O)OC(C)(C)C)CC2)ncc1C(=O)O ; Cc1cn2cc(N)ncc2n1 ; CCOc1nc(N(C)C2CCN(C(=O)OC(C)(C)C)CC2)ncc1C(=O)Nc1cn2cc(C)nc2cn1 ; CCOc1nc(N(C)C2CCNCC2)ncc1C(=O)Nc1cn2cc(C)nc2cn1]

### Step 1: Preparation of tert-butyl 4-((5-((2,8-dimethylimidazo[1,2-a]pyrazin-6-yl) carbamoyl)-4-ethoxypyrimidin-2-yl) (methyl) amino) piperidine-1-carboxylate

To a mixture of 2-((1-(tert-butoxycarbonyl) piperidin-4-yl) (methyl) amino)-4-ethoxypyrimidine-5-carboxylic acid (150 mg, 0.39 mmol) in DMF (5 mL) were added HATU (120 mg, 0.30 mmol), DIEA (136 mg, 1.00 mmol) and 2-methylimidazo[1,2-a]pyrazin-6-amine (46 mg, 0.30 mmol). The mixture was stirred at 45° C. for 16 h. The mixture was diluted with water (20 mL), extracted with EA (20 mL×3). The organic layer was washed with brine, dried over Na₂SO₄ and concentrated in vacuo to give title product (100 mg, 74.63%) as a yellow solid, which was used to next step without further purification. ESI-MS (M+H)⁺: 511.3.

### Step 2: Preparation of 4-ethoxy-2-(methyl(piperidin-4-yl) amino)-N-(2-methylimidazo[1,2-a]pyrazin-6-yl)pyrimidine-5-carboxamide

To a mixture of tert-butyl 4-((4-ethoxy-5-((2-methylimidazo[1,2-a]pyrazin-6-yl) carbamoyl)pyrimidin-2-yl) (methyl) amino) piperidine-1-carboxylate (100 mg, 0.20 mmol) in EA (3 mL) was added 3 M HCl/EA (3 mL). The mixture was stirred at rt for 2 h. The mixture was concentrated in vacuo. The residue was purified by prep-HPLC (0.05% NH₃·H₂O in water/CH₃CN) to give title product (12 mg, Y: 14.92%) as a yellow solid. ESI-MS (M+H⁺): 411.1. ¹H NMR (400 MHZ, MeOD-d₄)δ 9.30 (d, J=1.3 Hz, 1H), 8.87 (s, 1H), 8.70 (s, 1H), 7.85 (s, 1H), 4.68 (q, J=7.0 Hz, 2H), 3.48-3.47 (m, 1H), 3.18-3.17 (m, 2H), 3.13 (s, 3H), 2.75 (t, J=11.1 Hz, 2H), 2.47 (s, 3H), 1.88-1.79 (m, 2H), 1.75-1.74 (m, 2H), 1.57 (t, J=7.1 Hz, 3H).

### Example 37. 4-methoxy-2-(methyl(piperidin-4-yl) amino)-N-(2-methylimidazo[1,2-a]pyrazin-6-yl)pyrimidine-5-carboxamide formate (Compound 257)

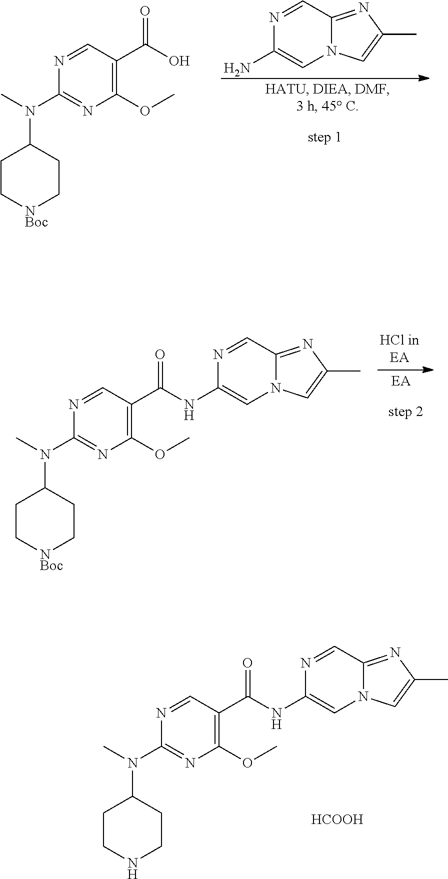 [SMILES: COc1nc(N(C)C2CCN(C(=O)OC(C)(C)C)CC2)ncc1C(=O)O ; Cc1cn2cc(N)ncc2n1 ; COc1nc(N(C)C2CCN(C(=O)OC(C)(C)C)CC2)ncc1C(=O)Nc1cn2cc(C)nc2cn1 ; COc1nc(N(C)C2CCNCC2)ncc1C(=O)Nc1cn2cc(C)nc2cn1 ; "HCOOH"]

### Step 1: Preparation of ter-butyl 4-(4-methoxy-5-(2-methylimidazo[1,2-a]pyrazin-6-yl) carbamoyl)pyrimidin-2-yl) (methyl) amino) piperidine-1-carboxylate

To a mixture of 2-((1-(tert-butoxycarbonyl) piperidin-4-yl) (methyl) amino)-4-methoxypyrimidine-5-carboxylic acid (50 mg, 0.137 mmol) in DMF (3 mL) were added HATU (62 mg, 0.164 mmol), DIEA (70 mg, 0.546 mmol) and 2-methylimidazo[1,2-a]pyrazin-6-amine (30 mg, 0.205 mmol). The mixture was stirred at 45° C. for 3 h. The reaction mixture was diluted with water (20 mL), extracted with EA (30 ml . . . x2), and the organic phase was washed with brine, dried over sodium sulfate and concentrated to give title product (50 mg, 68%) as a yellow solid. ESI-MS (M+H)⁺: 497.1.

### Step 2: Preparation of 4-methoxy-2-(methyl(piperidin-4-yl) amino)-N-(2-methylimidazo[1,2-a]pyrazin-6-yl)pyrimidine-5-carboxamide formate

To a mixture of tert-butyl 4-((4-methoxy-5-((2-methylimidazo[1,2-a]pyrazin-6-yl) carbamoyl)pyrimidin-2-yl) (methyl) amino) piperidine-1-carboxylate (50 mg, 0.101 mmol) in EA (2 mL) was added 3 M HCl/EA (2 mL). The mixture was stirred at RT for 2 h. The mixture was concentrated in vacuo, the residue was purified by prep-HPLC (0.05% FA in water/ACN) to give title product (3 mg, Y: 6.74%) as a yellow solid. ESI-MS (M+H)⁺: 397.1. ¹H NMR (400 MHZ, MeOD-d₄)δ 9.32 (s, 1H), 8.88 (s, 1H), 8.71 (s, 1H), 8.53 (s, 1H), 7.86 (s, 1H), 5.05-4.93 (m, 1H), 4.22 (s, 3H), 3.49-3.45 (m, 2H), 3.18 (s, 3H), 3.14-3.07 (m, 2H), 2.48 (s, 3H), 2.07-1.93 (m, 4H).

### Example 38. 4-ethoxy-2-(ethyl(piperidin-4-yl) amino)-N-(7-fluoro-2-methyl-2H-indazol-5-yl)pyrimidine-5-carboxamide formate (Compound 258)

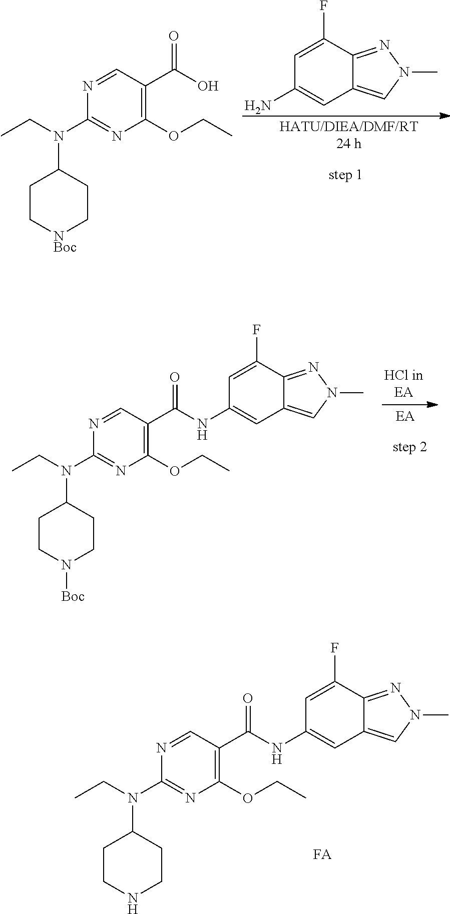

### Step 1: Preparation of tert-butyl 4-((+-ethoxy-5-((7-fluoro-2-methyl-2H-indazol-5-yl) carbamoyl)pyrimidin-2-yl) (ethyl) amino) piperidine-1-carboxylate

To a mixture of 2-((1-(tert-butoxycarbonyl) piperidin-4-yl) (ethyl) amino)-4-ethoxypyrimidine-5-carboxylic acid (50 mg. 0.13 mmol) in DMF (5 mL) were added 7-fluoro-2-methyl-2H-indazol-5-amine (21 mg, 0.13 mmol), HATU (145 mg, 0.38 mmol) and DIEA (82 mg, 0.64 mmol). The mixture was stirred at rt for 24 h. The mixture was diluted with H₂O (5 mL). The precipitate was filtered and dried in vacuo to give crude title product (50 mg, Y: 36.4%) as a yellow solid. ESI-MS (M+H)⁺: 542.2. ¹H NMR (400 MHZ, DMSO-d₆)δ 9.61 (s, 1H), 8.64 (s, 1H), 8.41 (d, J=2.7 Hz, 1H), 7.98 (s, 1H), 7.30 (d, J=13.3 Hz, 1H), 4.56-4.48 (m, 2H), 4.18 (s, 3H), 4.14-4.03 (m, 2H), 3.60-3.49 (m, 2H), 2.92-2.70 (m, 3H), 1.83-1.60 (m, 4H), 1.48-1.37 (m, 12H), 1.18 (s, 3H).

### Step 2: Preparation of 4-ethoxy-2-(ethyl(piperidin-4-yl) amino)-N-(7-fluoro-2-methyl-2H-indazol-5-yl)pyrimidine-5-carboxamide formate

A mixture of tert-butyl 4-((4-ethoxy-5-((7-fluoro-2-methyl-2H-indazol-5-yl) carbamoyl)pyrimidin-2-yl) (ethyl) amino) piperidine-1-carboxylate (100 mg, 0.19 mmol) in 3 M HCVEA (5 mL) was stirred at rt for 2 h. The mixture was diluted with H₂O (5 mL), extracted with EA (10 mL×3), the aqueous layer was concentrated in vacuo and the residue was purified by prep-HPLC (0.05% FA in water/ACN) to give title product (16 mg, 17.8%) as a yellow solid. ESI-MS (M+H⁺): 442.2. ¹H NMR (400 MHZ, MeOD-d₄)δ 8.80 (s, 1H), 8.54 (s, 1H), 8.25 (s, 1H), 7.88 (s, 1H), 7.23 (d, J=12.4 Hz, 1H), 4.85-4.79 (m, 1H), 4.69-4.61 (m, 2H), 4.22 (s, 3H), 3.69-3.61 (m, 2H), 3.50 (d, J=12.6 Hz, 2H), 3.12 (t, J=13.8 Hz, 2H), 2.21-2.05 (m, 2H), 2.05-1.97 (m, 2H), 1.56 (t, J=7.0 Hz, 3H), 1.32-1.24 (m, 3H).

### Example 39. 4-ethoxy-N-(7-fluoro-2-methyl-2H-indazol-5-yl)-2-(4-methylpiperazin-1-yl)pyrimidine-5-carboxamide formate (Compound 259)

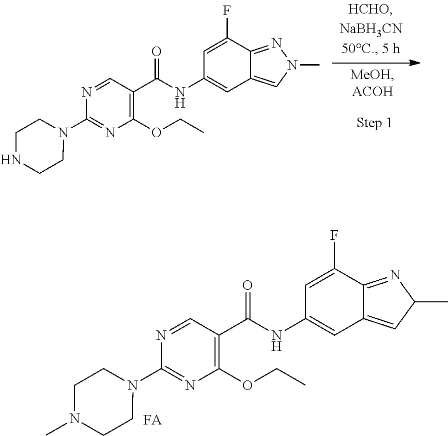

### Step 1: Preparation of 4-ethoxy-N-(7-fluoro-2-methyl-2H-indazol-5-yl)-2-(4-methylpiperazin-1-yl)pyrimidine-5-carboxamide formate

A mixture of 6-ethoxy-2-methyl-N-(6-(piperazin-1-yl) pyridazin-3-yl)-2H-pyrazolo[3,4-b]pyridine-5-carboxamide (50 mg, 0.13 mmol), AcOH (23 mg, 0.38 mmol) in MeOH (10 mL) were added NaBH₃CN (40 mg, 0.63 mmol) and (HCHO), (20 mg, 0.63 mmoL). The mixture was stirred at 50° C. for 5 h. Filtered and the filtrate was concentrated in vacuo. The residue was purified by prep-HPLC (0.05% FA in water/ACN) to give title product (16 mg, Y: 27.9%) as a yellow solid. ESI-MS (M+H⁺): 414.1. ¹H NMR (400 MHZ, MeOD-d₄)δ 8.80 (s, 1H), 8.32 (s, 1H), 8.26-8.23 (m, 1H), 7.88 (d, J=1.4 Hz, 1H), 7.22 (dd, J=12.7, 1.5 Hz, 1H), 4.63 (q, J=7.1 Hz, 2H), 4.22 (s, 3H), 4.06-3.99 (m, 4H), 2.85-2.80 (m, 4H), 2.57 (s, 3H), 1.55 (t, J=7.1 Hz, 3H).

### Example 40. 4-ethoxy-2-(ethyl(piperidin-4-yl) amino)-N-(8-methoxy-2-methylimidazo[1,2-a]pyridin-6-yl)pyrimidine-5-carboxamide formate (Compound 260)

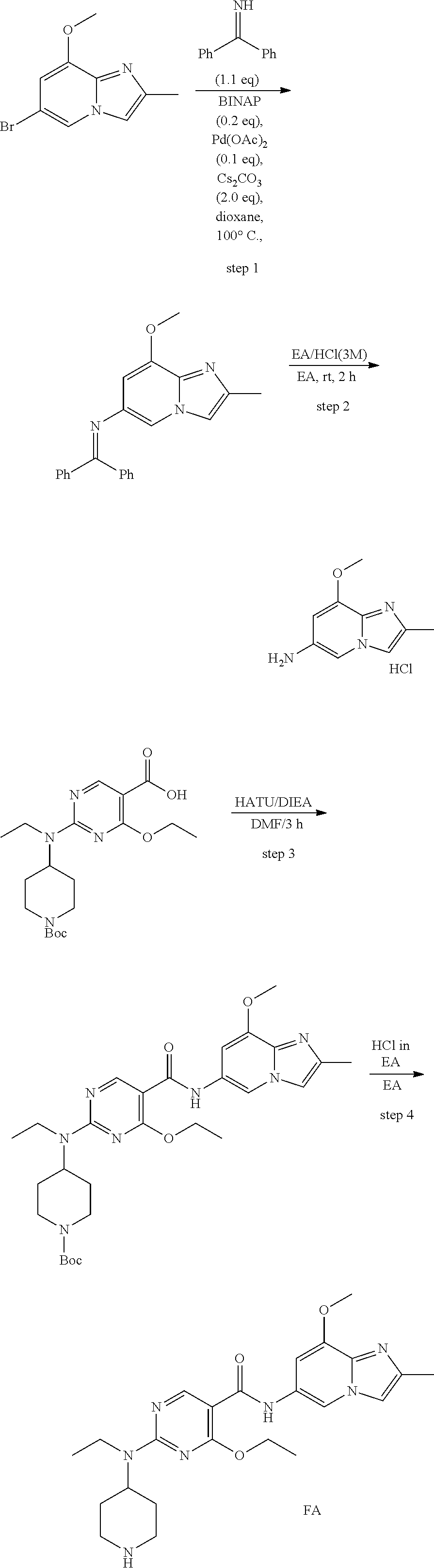

### Step 1: Preparation of N-(8-methoxy-2-methylimidazo[1,2-a]pyridin-6-yt)-1,1-diphenylmethanimine

To a mixture of 6-bromo-8-methoxy-2-methylimidazo[1,2-a]pyridine (6.8 g, 28.33 mmol) and diphenylmethanimine (5.6 g, 31.17 mmol) in 1,4-dioxane (250 mL) were added BINAP (3.53 g, 0.56 mmol), Pd(OAc): (636 mg, 0.28 mmol) and Cs₂CO₃ (18.5 g, 0.057 mol). The reaction solution was stirred at 100° C. for 16 h. The reaction mixture was diluted with H₂O (50 mL), extracted with EA (85 mL×3). The organic layer was washed with brine, dried over Na₂SO₄ and evaporated. The crude was purified by silica gel column chromatography (PE:EA=3:1) to give title product (6.8 g. 70.4%) as a yellow solid. ESI-MS (M+H)⁺: 342.2. ¹H NMR (400 MHZ, CDCl₃) δ 7.77-7.72 (m, 2H), 7.51-7.46 (m, 1H), 7.44-7.39 (m, 2H), 7.35-7.29 (m, 3H), 7.23 (d, J=1.6 Hz, 1H), 7.20-7.16 (m, 2H), 7.14-7.10 (m, 1H), 5.96 (d, J=1.5 Hz, 1H), 3.71 (s, 3H), 2.38 (s, 3H).

### Step 2: Preparation of 7-fluoro-2-methyl-2H-indazol-5-amine hydrochloride

A mixture of N-(8-methoxy-2-methylimidazo[1,2-a]pyridin-6-yl)-1,1-diphenylmethanimine (6.8 g, 19.88 mmol) in 3M HCl/EA (80 mL) was stirred at rt for 2 h. The reaction was diluted with H₂O (30 mL), extracted with EA (30 mL). The aqueous layer was purified by reversed phase column (0.05% NH₃.H₂O in water/CH₃CN) to give title product (4.2 g, 98.8%) as a yellow solid. ESI-MS (M+H)⁺: 178.2. ¹H NMR (400 MHZ, DMSO-d₆) δ 8.04-7.92 (m, 1H), 7.88-7.64 (m, 1H), 7.15-6.95 (m, 1H), 4.01 (s, 3H), 2.39 (s, 3H).

### Step 3: Preparation of ter-butyl 4-((4-ethoxy-5-(8-methoxy-2-methylimidazo[1,2-a]pyridin-6-yl) carbamoyl)pyrimidin-2-yl) (ethyl) amino) piperidine-1-carboxylate

To a mixture of 2-((1-(tert-butoxycarbonyl) piperidin-4-yl) (ethyl) amino)-4-ethoxypyrimidine-S-carboxylic acid (50 mg, 0.12 mmol) in DMF (5 mL) were added 7-fluoro-2-methyl-2H-indazol-5-amine hydrochloride (45 mg, 0.25 mmol), HATU (145 mg 0.38 mmol) and DIEA (82 mg, 0.63 mmol). The mixture was stirred at rt for 3 h. The mixture was poured into H₂O) (5 mL), the precipitate was filtered and concentrated in vacuo to give crude title product (30 mg 42.7%) as a yellow solid. ESI-MS (M+H)⁺: 554.2.

### Step 4: Preparation of 4-ethoxy-2-(ethyl(piperidin-4-yl) amino)-N-(8-methoxy-2-methylimidazo[1,2-a]pyridin-6-yl)pyrimidine-5-carboxamide formate

A mixture of tert-butyl 4-((4-ethoxy-5-((8-methoxy-2-methylimidazo[1,2-a]pyridin-6-yl) carbamoyl)pyrimidin-2-yl) (ethyl) amino) piperidine-1-carboxylate (60 mg, 0.11 mmol) in 3M HCl/EA (3 mL) was stirred at rt for 1 h. The mixture was diluted with H₂O (5 mL), extracted with EA (10 mL). The aqueous layer was concentrated in vacuo and purified by prep-HPLC (0.05% FA in water/ACN) to give title product (9 mg, 16.6%) as a yellow solid. ESI-MS (M+H)⁺: 454.2. 1 H NMR (400 MHZ, DMSO-d₆) δ 9.54 (s, 1H), 8.82 (s, 1H), 8.63 (s, 1H), 8.29 (s, 1H), 7.69 (s, 1H), 6.66 (s, 1H), 5.00-4.46 (m, 3H), 3.92 (s, 3H), 3.65-3.47 (m, 2H), 3.33 (d, J=10.7 Hz, 2H), 2.97 (t, J=12.0 Hz, 2H), 2.29 (s, 3H), 2.03 (d, J=11.1 Hz, 2H), 1.88-1.73 (m, 2H), 1.45 (t, J=7.0 Hz, 3H), 1.20 (s, 3H).

### Example 41. (S)-4-ethoxy-N-(8-fluoro-2-methylimidazo[1,2-a]pyridin-6-yl)-2-(3-(methylamino) pyrrolidin-1-yl)pyrimidine-5-carboxamide formate (Compound 261)

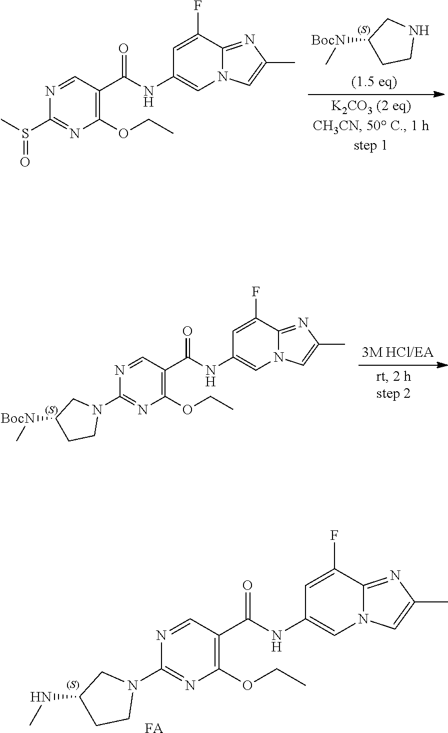

### Step 1: Preparation of ter-butyl(S)-(1-(4-ethoxy-5-(8-fluoro-2-methylimidazo[1,2-a]pyridin-6-yl) carbamoyl)pyrimidin-2-yl) pyrrolidin-3-yl) (methyl) carbamate

To a mixture of 4-ethoxy-N-(8-fluoro-2-methylimidazo[1,2-a]pyridin-6-yl)-2-(methylsulfinyl)pyrimidine-5-carboxamide (500 mg, 1.33 mmol) in MeCN (15 mL) were added K₂CO₃ (366 mg. 2.66 mmol) and tert-butyl(S)-methyl(pyrrolidin-3-yl) carbamate (400 mg, 2.00 mmol). The mixture was stirred at 50° C. for 1 h. The mixture was filtered and the filtrate was concentrated. The residue was purified by silica gel column chromatography (PE: EA=1:2) to give title product (400 mg, 58.7%) as an off-white solid. ESI-MS (M+H)⁺: 514.2.

### Step 2: Preparation of(S)-4-ethoxy-N-(8-fluoro-2-methylimidazo[1,2-a]pyridin-6-yl)-2-(3-(methylamino) pyrrolidin-1-yl)pyrimidine-5-carboxamide formate

A mixture of tert-butyl(S)-(1-(4-ethoxy-5-((8-fluoro-2-methylimidazo[1,2-a]pyridin-6-yl) carbamoyl)pyrimidin-2-yl) pyrrolidin-3-yl) (methyl) carbamate (400 mg, 0.78 mmol) in 3M HCVEA (15 mL) was stirred at rt for 2 h. The mixture was concentrated in vacuo, the residue was purified by prep-HPLC (0.05% FA in water/ACN) to give title product (250 mg, Y: 69%) as an off-white solid. ESI-MS (M+H⁺): 414.1. ¹H NMR (400 MHZ, DMSO-d₆) δ 9.55 (s, 1H), 9.06 (d, J=1.1 Hz, 1H), 8.63 (s, 1H), 8.30 (s, 1H), 7.87 (d, J=2.6 Hz, 1H), 7.20 (dd, J=12.5, 1.2 Hz, 1H), 4.60-4.49 (m, 2H), 3.75-3.64 (m, 2H), 3.61-3.44 (m, 3H), 2.42 (s, 3H), 2.33 (s, 3H), 2.22-2.12 (m, 1H), 2.03-1.90 (m, 1H), 1.45 (t, J=7.0 Hz, 3H).

### Example 42. (R)-4-ethoxy-N-(8-fluoro-2-methylimidazo[1,2-a]pyridin-6-yl)-2-(3-(methylamino) pyrrolidin-1-yl)pyrimidine-5-carboxamide formate (Compound 262)

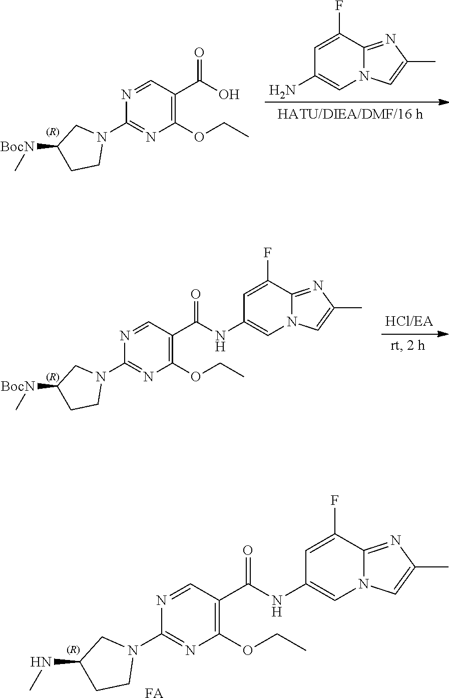

### Step 1: Preparation of tert-butyl(R)-(1-(4-ethoxy-5-((8-fluoro-2-methylimidazo[1,2-a]pyridin-6-yl) carbamoyl)pyrimidin-2-yl) pyrrolidin-3-yl) (methyl) carbamate

To a mixture of (R)-2-(3-((tert-butoxycarbonyl) (methyl) amino) pyrrolidin-1-yl)-4-ethoxypyrimidine-5-carboxylic acid (500 mg, 1.37 mmol) in DMF (5 mL) were added 8-fluoro-2-methylimidazo[1,2-a]pyridin-6-amine (225 mg, 1.37 mmol), HATU (779 mg, 2.05 mmol) and DIEA (881 mg, 6.83 mmol). The mixture was stirred at rt for 16 h. The mixture was diluted with water. The precipitate was filtered and dried in vacuo to give crude title product (500 mg, Y: 71.3%) as a yellow solid. ESI-MS (M+H): 514.1

### Step 2: Preparation of (R)-4-ethoxy-N-(8-fluoro-2-methylimidazo[1,2-a]pyridin-6-yl)-2-(3-(methylamino) pyrrolidin-1-yl)pyrimidine-5-carboxamide formate

A mixture of tert-butyl(R)-(1-(4-ethoxy-5-((8-fluoro-2-methylimidazo[1,2-a]pyridin-6-yl) carbamoyl)pyrimidin-2-yl) pyrrolidin-3-yl) (methyl) carbamate (500 mg, 0.98 mmol) in 3 M HCl/EA (5 mL) was stirred at rt for 2 h. The mixture was diluted with H₂O (5 mL), extracted with EA (10 mL×3). The aqueous layer was concentrated in vacuo and the residue was purified by prep-HPLC (0.05% FA in water/ACN) to give title product (300 mg, Y: 67.1%) as a yellow solid. ESI-MS (M+H⁺): 414.1. 1 H NMR (400 MHZ, DMSO-d₆) δ 9.57 (s, 1H), 9.07 (s, 1H), 8.64 (s, 1H), 8.25 (s, 1H), 7.88 (d, J=2.7 Hz, 1H), 7.22 (d, J=12.5 Hz, 1H), 4.55 (q, J=7.0 Hz, 2H), 3.77-3.64 (m, 2H), 3.61-3.49 (m, 3H), 2.43 (s, 3H), 2.33 (s, 3H), 2.22-2.13 (m, 1H), 2.04-1.93 (m, 1H), 1.44 (t, J=7.0 Hz, 3H).

### Example 43. 4-ethoxy-N-(7-fluoro-2-methyl-2H-indazol-5-yl)-2-(3-(methylamino) pyrrolidin-1-yl)pyrimidine-5-carboxamide formate (Compound 263)

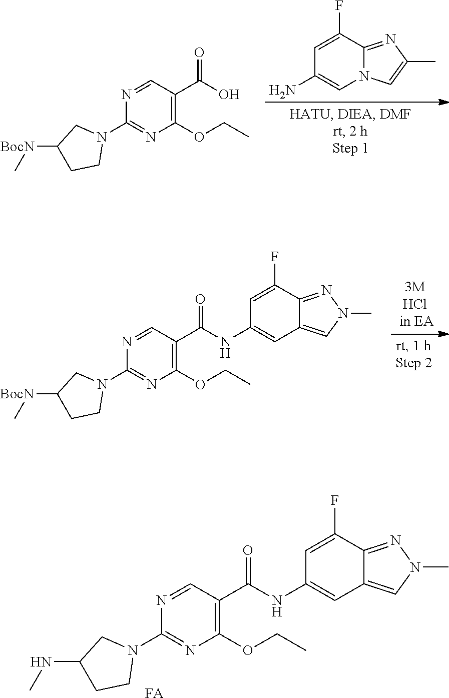

### Step 1: Preparation of tert-butyl(1-(4-ethoxy-5-((7-fluoro-2-methyl-2H-indazol-5-yl) carbamoyl)pyrimidin-2-yl) pyrrolidin-3-yl) (methyl) carbamate

To a mixture of 2-(3-((tert-butoxycarbonyl) (methyl) amino) pyrrolidin-1-yl)-4-ethoxypyrimidine-5-carboxylic acid (100 mg, 0.27 mmol) in DMF (3 mL) were added 7-fluoro-2-methyl-2H-indazol-5-amine (45 mg, 0.27 mmol), HATU (155 mg, 0.4 mmol) and DIEA (176 mg, 1.35 mmol). The mixture was stirred at rt for 2 h. The mixture was diluted with H₂O (5 mL) and the precipitate was filtered and dried in vacuo to give title product (80 mg, crude) as a yellow solid. ESI-MS (M+H)⁺: 514.3.

### Step 2: Preparation of 4-ethoxy-N-(7-fluoro-2-methyl-2H-indazol-5-yl)-2-(3-(methylamino) pyrrolidin-1-yl)pyrimidine-5-carboxamide formate

A mixture of tert-butyl(1-(4-ethoxy-5-((7-fluoro-2-methyl-2H-indazol-S-yl) carbamoyl)pyrimidin-2-yl) pyrrolidin-3-yl) (methyl) carbamate (80 mg, 0.16 mmol) in 3M HCl/EA (3 mL) was stirred at rt for 1 h. The mixture was concentrated in vacuo and the residue was purified by prep-HPLC (0.05% FA in water/ACN) to give title product (12 mg, Y: 16.4%) as a yellow solid. ESI-MS (M+HT): 414.5. ¹H NMR (400 MHZ, MeOD-d₄)δ 8.80 (s, 1H), 8.47 (s, 2H), 8.25 (d, J=2.6 Hz, 1H), 7.89 (s, 1H), 7.23 (d, J=12.8 Hz, 1H), 4.67 (q, J=7.1 Hz, 2H), 4.22 (s, 3H), 4.02-3.95 (m, 1H), 3.90-3.73 (m, 4H), 2.74 (s, 3H), 2.52-2.43 (m, 1H), 2.26-2.16 (m, 1H), 1.56 (t, J=7.1 Hz, 3H).

### Example 44. (R)-4-ethoxy-N-(7-fluoro-2-methyl-2H-indazol-5-yl)-2-(3-methylpiperazin-1-yl)pyrimidine-5-carboxamide formic acid (Compound 264)

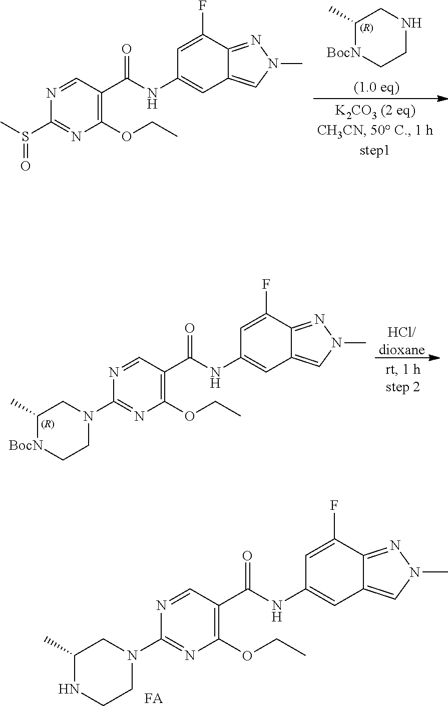

### Step 1: tert-butyl(R)-4-(4-ethoxy-5-((7-fluoro-2-methyl-2H-indazol-5-yl) carbamoyl)pyrimidin-2-yl)-2-methylpiperazine-1-carboxylate

To a solution of 4-ethoxy-N-(7-fluoro-2-methyl-2H-indazol-5-yl)-2-(methylsulfinyl)pyrimidine-5-carboxamide (300.0 mg, 0.794 mmol) and tert-butyl(R)-2-methylpiperazine-1-carboxylate (160.0 mg, 0.80 mmol) in CH₃CN (5 mL) was added K₂CO₃ (219.0 mg, 1.588 mmol), the reaction mixture was stirred at 50° C. for 1 hour. The mixture was diluted with EA (20 mL), washed with water (10 mL) and brine (20 mL), dried over sodium sulfate, filtered and concentrated in vacuum to afford the crude title product (300.0 mg, yield: 73.4%) as a yellow solid. ESI-MS (M+H)⁺: 514.3.

### Step 2: (R)-4-ethoxy-N-(7-fluoro-2-methyl-2H-indazol-5-yl)-2-(3-methylpiperazin-1-yl)pyrimidine-5-carboxamide formic acid

To a solution of tert-butyl(R)-4-(4-ethoxy-5-((7-fluoro-2-methyl-2H-indazol-5-yl) carbamoyl)pyrimidin-2-yl)-2-methylpiperazine-1-carboxylate (100 mg, 0.194 mmol) in DCM (2 mL) was added 4M HCl in 1,4-dioxane (1 mL), the mixture was stirred at room temperature for 2 hours. The precipitate was filtered and concentrated in vacuum. The residue was purified by C₁₈ reverse chromatography with 0.05% FA in water (from 0 to 100% in 40 min gradient) to afford title product (10.53 mg, yield: 12.5%) as a yellow solid. ¹H NMR (400 MHZ, DMSO-d₆) § 9.59 (s, 1H), 8.63 (s, 1H), 8.41 (d, J=2.4 Hz, 1H), 8.21 (s, 1H), 7.97 (s, 1H), 7.28 (d, J=13.1 Hz, 1H), 4.54-4.48 (m, 4H), 4.17 (s, 3H), 2.97-2.93 (m, 2H), 2.77-2.55 (m, 3H), 1.44 (t, J=7.0 Hz, 3H), 1.05 (d, J=5.9 Hz, 3H) ESI-MS: [M+H]+=414.1.

### Example 45. 2-(3,3-dimethylpiperazin-1-yl)-4-ethoxy-N-(7-fluoro-2-methyl-2H-indazol-5-yl)pyrimidine-5-carboxamide (Compound 265)

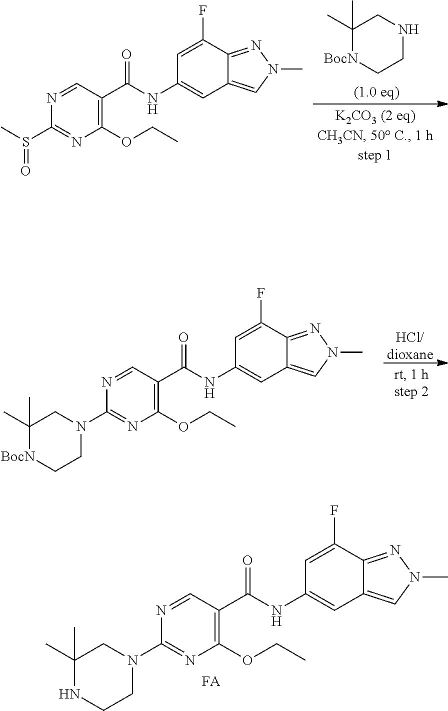

### Step 1: tert-butyl 4-(4-ethoxy-5-(7-fluoro-2-methyl-2H-indazol-5-yl) carbamoyl)pyrimidin-2-yl)-2,2-dimethylpiperazine-1-carboxylate

To a solution of 4-ethoxy-N-(7-fluoro-2-methyl-2H-indazol-5-yl)-2-(methylsulfinyl)pyrimidine-5-carboxamide (140.0 mg, 0.371 mmol) and tert-butyl 2,2-dimethylpiperazine-1-carboxylate (94.0 mg, 0.43 mmol) in CH₃CN (5 mL) was added K₂CO₃ (120.0 mg, 1.2 mmol), the reaction mixture was stirred at 50° C. for 1 hours. The mixture was diluted with EA (20 mL), washed with water (10 mL) and brine (20 mL), dried over sodium sulfate, filtered and concentrated in vacuum to afford the crude title product (130.0 mg, 66.42%) as a yellow solid. The crude product was used to next step without further purification. ESI-MS (M+H)⁺: 528.3.

### Step 2:2-(3,3-dimethylpiperazin-1-yl)-4-ethoxy-N-(7-fluoro-2-methyl-2H-indazol-5-yl)pyrimidine-5-carboxamide

To a solution of tert-butyl 4-(4-ethoxy-5-((7-fluoro-2-methyl-2H-indazol-5-yl) carbamoyl)pyrimidin-2-yl)-2,2-dimethylpiperazine-1-carboxylate (130 mg, 0.246 mmol) in DCM (2 mL) was added 4M HCl in 1,4-dioxane (2 mL), the mixture was stirred at room temperature for 2 hours. The mixture was concentrated in vacuum. The residue was purified by C₁₈ reverse chromatography with 0.01% FA in water (from 0 to 100% in 40 min gradient) to afford title product (16.54 mg, yield: 15.57%) as a yellow solid. ¹H NMR (400 MHZ, DMSO-d₆) δ 9.60 (s, 1H), 8.62 (d, J=6.7 Hz, 1H), 8.41 (d, J=2.7 Hz, 1H), 8.20 (s, 1H), 7.96 (s, 1H), 7.28 (d, J=13.3 Hz, 1H), 4.49 (q, J=6.7 Hz, 2H), 4.17 (s, 3H), 3.80 (br s, 2H), 3.64 (s, 3H), 2.87 (br s, 2H), 1.43 (t, J=7.0 Hz, 3H), 1.08 (s, 6H). ESI-MS: [M+H]⁺: 428.2.

### Example 46. N-(2,8-dimethylimidazo[1,2-a]pyrazin-6-yl)-4-methoxy-2-(piperidin-4-ylamino) pyrimidine-5-carboxamide formic acid (Compound 266)

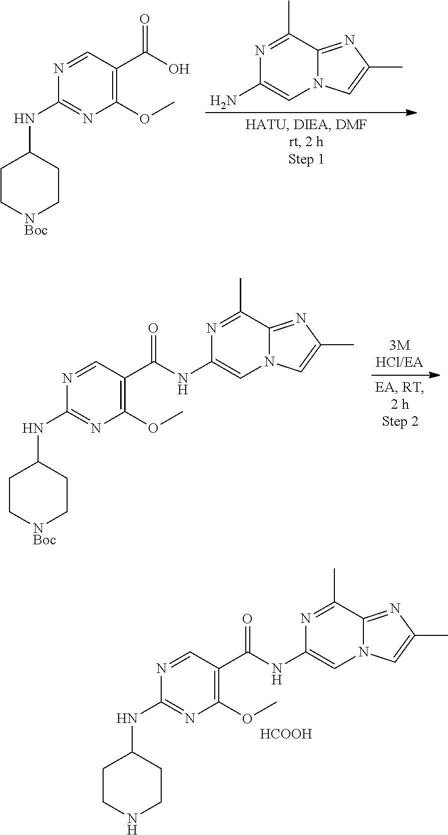 [SMILES: COc1nc(NC2CCN(C(=O)OC(C)(C)C)CC2)ncc1C(=O)O ; Cc1cn2cc(N)nc(C)c2n1 ; COc1nc(NC2CCN(C(=O)OC(C)(C)C)CC2)ncc1C(=O)Nc1cn2cc(C)nc2c(C)n1 ; COc1nc(NC2CCNCC2)ncc1C(=O)Nc1cn2cc(C)nc2c(C)n1 ; "HCOOH"]

### Step 1: Preparation of ter-butyl 4-(5-(2,8-dimethylimidazo[1,2-a]pyrazin-6-yl) carbamoyl)-4-methoxypyrimidin-2-yl) amino) piperidine-1-carboxylate

To a mixture of 2-((1-(tert-butoxycarbonyl) piperidin-4-yl)amino)-4-methoxypyrimidine-5-carboxylic acid (100 mg, 0.284 mmol) in DMF (3 mL) were added HATU (130 mg, 0.341 mmol), DIEA (147 mg, 1.136 mmol) and 2,8-dimethylimidazo[1,2-a]pyrazin-6-amine (76 mg, 0.426 mmol). The mixture was stirred at 45° C. for 3 h. The reaction mixture was diluted with H₂O (20 mL), extracted with EA (30 mL×2). The organic phase was washed with brine, dried over sodium sulfate and concentrated to give title product (50 mg, 35%) as a brown solid, which was used to next step without further purification. ESI-MS (M+H)⁺: 497.3.

### Step 2: Preparation of N-(2,8-dimethylimidazo[1,2-a]pyrazin-6-yl)-4-methoxy-2-(piperidin-4-ylamino) pyrimidine-5-carboxamide formic acid

To a mixture of tert-butyl 4-((5-((2,8-dimethylimidazo[1,2-a]pyrazin-6-yl) carbamoyl)-4-methoxypyrimidin-2-yl) amino) piperidine-1-carboxylate (50 mg, 0.101 mmol) in EA (2 mL) was added EA/HCl (2 mL). The mixture was stirred at RT for 2 h. The mixture was concentrated in vacuo, the residue was purified by prep-HPLC (0.05% FA in water/ACN) to give title product (9 mg, Y: 20%) as a white solid. ESI-MS (M+H)⁺: 397.1. ¹H NMR (400 MHZ, MeOD-d₄)δ 9.12 (s, 1H), 8.80 (s, 1H), 8.53 (s, 1H, HCO2H), 7.76 (s, 1H), 4.22-4.15 (m, 3H), 3.54-3.38 (m, 3H), 3.20-3.11 (m, 2H), 2.75 (s, 3H), 2.45 (s, 3H), 2.30-2.21 (m, 2H), 1.88-1.75 (m, 2H).

### Example 47. 4-ethoxy-2-(ethyl(piperidin-4-yl) amino)-N-(2-methylimidazo[1,2-a]pyrazin-6-yl)pyrimidine-5-carboxamide formic acid (Compound 267)

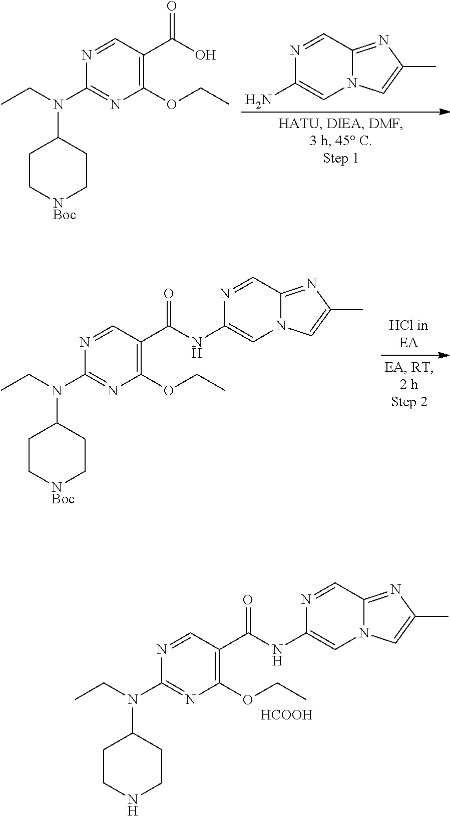

### Step 1: Preparation of tert-butyl 4-((4-ethoxy-5-((2-methylimidazo[1,2-a]pyrazin-6-yl) carbamoyl)pyrimidin-2-yl) (ethyl) amino) piperidine-1-carboxylate

To a mixture of 2-((1-(tert-butoxycarbonyl) piperidin-4-yl) (ethyl) amino)-4-ethoxypyrimidine-5-carboxylic acid (100 mg, 0.253 mmol) in DMF (3 mL) were added HATU (116 mg, 0.304 mmol), DIEA (131 mg, 1.02 mmol) and 2-methylimidazo[1,2-a]pyrazin-6-amine (56 mg, 0.381 mmol). The mixture was stirred at 45° C. for 3 h. The reaction was diluted with H₂O (20 mL), extracted with EA (30 mL×2). The organic phase was washed with brine, dried over sodium sulfate and concentrated to give title product (100 mg, 75%) as a brown solid, which was used to next step without further purification. ESI-MS (M+H)⁺: 525.3.

### Step 2: Preparation of 4-ethoxy-2-(ethyl(piperidin-4-yl) amino)-N-(2-methylimidazo[1,2-a]pyrazin-6-yl)pyrimidine-5-carboxamide formic acid

To a mixture of tert-butyl 4-((4-ethoxy-5-((2-methylimidazo[1,2-a]pyrazin-6-yl) carbamoyl)pyrimidin-2-yl) (ethyl) amino) piperidine-1-carboxylate (100 mg, 0.191 mmol) in EA (3 mL) was added 3M HCl/EA (3 mL). The mixture was stirred at RT for 2 h. The mixture was concentrated in vacuo, the residue was purified by prep-HPLC (0.05% FA in water/ACN) to give title product (18 mg, Y: 22%) as a yellow solid. ESI-MS (M+H)⁺: 425.1. ¹H NMR (400 MHZ, DMSO-d₆) δ 9.79 (s, 1H), 9.32 (s, 1H), 8.82-8.80 (m, 2H), 8.34 (s, 1H), 8.02 (s, 1H), 4.94-4.69 (m, 1H), 4.59 (q, J=7.0 Hz, 2H), 3.61-3.52 (m, 2H), 3.25-3.16 (m, 2H), 2.77 (t, J=11.6 Hz, 2H), 2.40 (s, 3H), 1.90-1.69 (m, 4H), 1.48 (t, J=7.0 Hz, 3H), 1.24-1.13 (m, 3H).

### Example 48. 4-ethoxy-N-(2-methylimidazo[1,2-a]pyrazin-6-yl)-2-(piperidin-4-ylamino) pyrimidine-5-carboxamide formate (Compound 268)

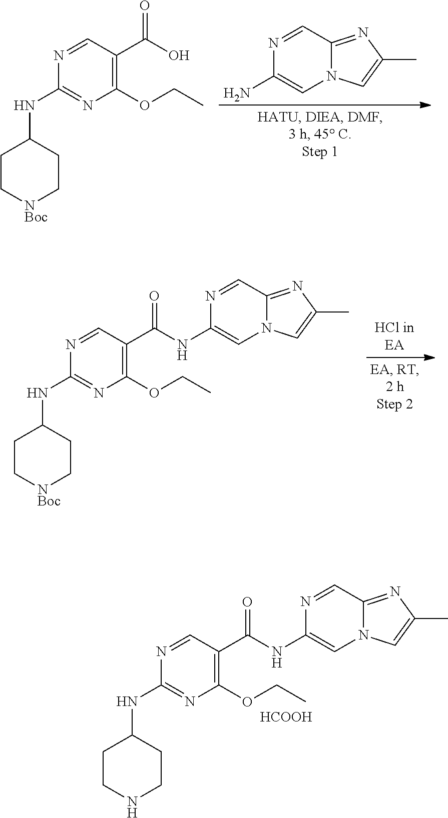

### Step 1: Preparation of tert-butyl 4-((4-ethoxy-5-((2-methylimidazo[1,2-a]pyrazin-6-yl) carbamoyl)pyrimidin-2-yl) amino) piperidine-1-carboxylate

To a mixture of 2-((1-(tert-butoxycarbonyl) piperidin-4-yl)amino)-4-ethoxypyrimidine-5-carboxylic acid (100 mg, 0.273 mmol) in DMF (3 mL) were added HATU (125 mg, 0.328 mmol), DIEA (141 mg, 1.09 mmol) and 2-methylimidazo[1,2-a]pyrazin-6-amine (61 mg, 0.410 mmol). The mixture was stirred at 45° C. for 3 h. The reaction mixture was diluted with H₂O (20 mL), extracted with EA (30 ml×2). The organic phase was washed with brine, dried over sodium sulfate and concentrated to give title product (50 mg, 37%) as a brown solid. ESI-MS (M+H)⁺: 497.3.

### Step 2: Preparation of 4-ethoxy-N-(2-methylimidazo[1,2-a]pyrazin-6-yl)-2-(piperidin-4-ylamino) pyrimidine-5-carboxamide formate

To a mixture of tert-butyl 4-((4-ethoxy-5-((2-methylimidazo[1,2-a]pyrazin-6-yl) carbamoyl)pyrimidin-2-yl) amino) piperidine-1-carboxylate (50 mg, 0.1 mmol) in EA (2 mL) was added 3M HCl/EA (2 mL). The mixture was stirred at RT for 2 h. The mixture was concentrated in vacuo, the residue was purified by prep-HPLC (0.05% FA in water/ACN) to give title product (8 mg, Y: 14.3%) as a white solid. ESI-MS (M+H)⁺: 397.1. ¹H NMR (400 MHz, MeOD-d₄) δ 9.32 (s, 1H), 8.83 (s, 1H), 8.72 (s, 1H), 8.52 (s, 1H), 7.89 (s, 1H), 4.18-4.11 (m, 1H), 3.46-3.36 (m, 4H), 3.13-3.06 (m, 2H), 2.47 (s, 3H), 2.24-2.18 (m, 2H), 1.83-1.75 (m, 2H), 1.58-1.53 (m, 3H).

### Example 49. 2-(3-(dimethylamino) pyrrolidin-1-yl)-4-ethoxy-N-(8-fluoro-2-methylimidazo[1,2-a]pyridin-6-yl)pyrimidine-5-carboxamide (Compound 269)

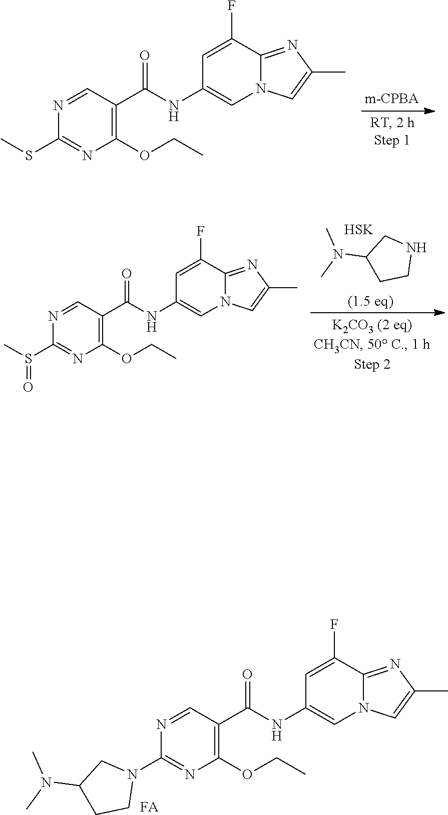

### Step 1: Preparation of 4-ethoxy-N-(8-fluoro-2-methylimidazo[1,2-a]pyridin-6-yl)-2-(methylsulfinyl)pyrimidine-5-carboxamide

To a mixture of 4-ethoxy-N-(8-fluoro-2-methylimidazo[1,2-a]pyridin-6-yl)-2-(methylthio)pyrimidine-5-carboxamide (200 mg, 0.55 mmol) in DCM (8 mL) was added m-CPBA (191.7 mg, 1.11 mmol). The mixture was stirred at 25° C. for 2 h. The reaction was added H₂O (10 mL), extracted with DCM (10 mL×3). The organic phase was concentrated to give title product (200 mg, 96%) as a yellow solid. ESI-MS (M+H)⁺: 378.0.

### Step 2: Preparation of 2-(3-(dimethylamino) pyrrolidin-1-yl)-4-ethoxy-N-(8-fluoro-2-methylimidazo[1,2-a]pyridin-6-yl)pyrimidine-5-carboxamide

To a mixture of 4-ethoxy-N-(8-fluoro-2-methylimidazo[1,2-a]pyridin-6-yl)-2-(methylsulfinyl)pyrimidine-5-carboxamide (177 mg, 0.47 mmol) and 4-ethoxy-N-(8-fluoro-2-methylimidazo[1,2-a]pyridin-6-yl)-2-(methylthio)pyrimidine-5-carboxamide (80 mg, 0.70 mmol) in ACN (15 mL) was added K₂CO₃ (129 mg, 0.94 mmol). The mixture was stirred at 50° C. for 1 h. The mixture was filtered and the filtrate was purified by prep-HPLC (0.05% FA in water/ACN) to give title product (13.5 mg, Y: 8%) as a yellow solid. ESI-MS (M+H⁺): 428.1. ¹H NMR (400 MHZ, MeOD-d₄)δ 8.88 (s, 1H), 8.65 (s, 1H), 8.44 (s, 1H), 7.63 (s, 1H), 6.93 (d, J=11.8 Hz, 1H), 4.60-4.49 (m, 2H), 4.04-3.87 (m, 1H), 3.84-3.71 (m, 1H), 3.68-3.35 (m, 3H), 2.69 (s, 6H), 2.40-2.36 (m, 4H), 2.11-2.10 (m, 1H), 1.54 (t, J=6.8 Hz, 3H).

### Example 50. 4-ethoxy-N-(2-methyl-2H-indazol-5-yl)-2-(3-(methylamino) pyrrolidin-1-yl)pyrimidine-5-carboxamide hydrochloride (Example 270)

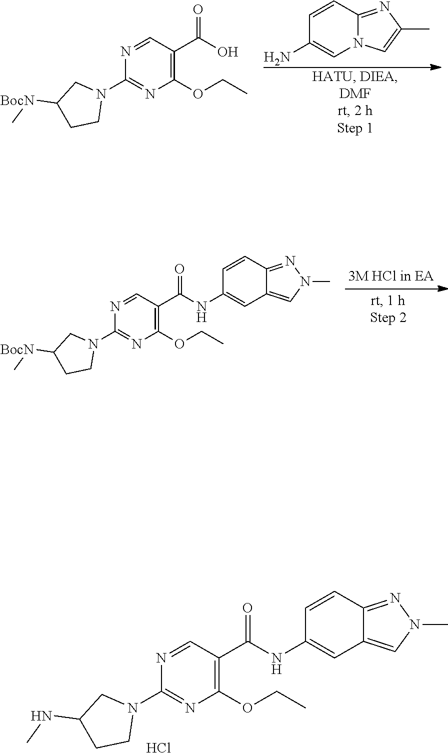

### Step 1: Preparation of tert-butyl(1-(4-ethoxy-5-(2-methyl-2H-indazol-5-yl) carbamoyl)pyrimidin-2-yl) pyrrolidin-3-yl) (methyl) carbamate

To a mixture of 2-(3-((tert-butoxycarbonyl) (methyl) amino) pyrrolidin-1-yl)-4-ethoxypyrimidine-5-carboxylic acid (100 mg, 0.28 mmol) in DMF (3 mL) were added 2-methyl-2H-indazol-5-amine (100 mg, 0.68 mmol), HATU (380 mg 1.00 mmol) and DIEA (440 mg, 3.4 mmol). The mixture was stirred at rt for 2 h. The mixture was diluted with H₂O (5 mL) and the precipitate was filtered and dried in vacuo to give title product (90 mg, crude) as a yellow solid. ESI-MS (M+H)⁺: 496.4.

### Step 2: Preparation of 4-ethoxy-N-(2-methyl-2H-indazol-5-yl)-2-(3-(methylamino) pyrrolidin-1-yl)pyrimidine-5-carboxamide hydrochloride

A mixture of tert-butyl(1-(4-ethoxy-5-((2-methyl-2H-indazol-5-yl) carbamoyl)pyrimidin-2-yl) pyrrolidin-3-yl) (methyl) carbamate (90 mg, 0.18 mmol) in 3M HCl/EA (3 mL) was stirred at rt for 1 h. The mixture was concentrated in vacuo and the residue was purified by prep-HPLC (0.05% HCl in water/ACN) to give title product (64 mg, Y: 82.7%) as a yellow solid. ESI-MS (M+H)⁺: 396.1. ¹H NMR (400 MHZ, DMSO-d₆) δ 9.60 (s, 1H), 9.27-9.09 (m, 2H), 8.69 (s, 1H), 8.29-8.21 (m, 2H), 7.58 (d, J=9.2 Hz, 1H), 7.29 (d, J=9.3 Hz, 1H), 4.58 (q, J=7.0 Hz, 2H), 4.14 (s, 3H), 3.93-3.85 (m, 2H), 3.82-3.75 (m, 2H), 3.65-3.64 (m, 1H), 2.65-2.61 (m, 3H), 2.38-2.32 (m, 1H), 2.28-2.19 (m, 1H), 1.47 (t, J=7.0 Hz, 3H).

### Example 51. 4-methoxy-N-(2-methylimidazo[1,2-a]pyrazin-6-yl)-2-(piperidin-4-ylamino) pyrimidine-5-carboxamide 2,2,2-trifluoroacetate (Compound 271)

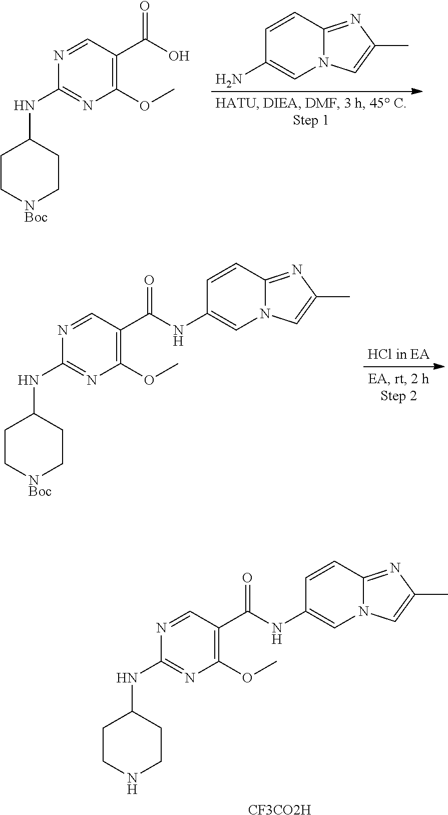 [SMILES: COc1nc(NC2CCN(C(=O)OC(C)(C)C)CC2)ncc1C(=O)O ; Cc1cn2cc(N)ccc2n1 ; COc1nc(NC2CCN(C(=O)OC(C)(C)C)CC2)ncc1C(=O)Nc1ccc2nc(C)cn2c1 ; COc1nc(NC2CCNCC2)ncc1C(=O)Nc1ccc2nc(C)cn2c1 ; "CF3CO2H"]

### Step 1: Preparation of tert-butyl 4-((4-methoxy-5-((2-methylimidazo[1,2-a]pyrazin-6-yl) carbamoyl)pyrimidin-2-yl) amino) piperidine-1-carboxylate

To a mixture of 2-((1-(tert-butoxycarbonyl) piperidin-4-yl)amino)-4-methoxypyrimidine-S-carboxylic acid (100 mg, 0.284 mmol) in DMF (3 mL) were added HATU (130 mg, 0.341 mmol), DIEA (147 mg, 1.14 mmol) and 2-methylimidazo[1,2-a]pyrazin-6-amine (63 mg, 0.426 mmol). The reaction mixture was diluted with H₂O (20 mL), extracted with EA (30 mL×2). The organic phase was washed with brine, dried over sodium sulfate and concentrated to give title product (100 mg, 73%) as a yellow solid. ESI-MS (M+H)⁺: 483.2

### Step 2: Preparation of 4-methoxy-N-(2-methylimidazo[1,2-a]pyrazin-6-yl)-2-(piperidin-4-ylamino) pyrimidine-5-carboxamide 2,2,2-trifluoroacetate

A mixture of tert-butyl 4-((4-methoxy-5-((2-methylimidazo[1,2-a]pyrazin-6-yl) carbamoyl)pyrimidin-2-yl) amino) piperidine-1-carboxylate (100 mg, 0.207 mmol) in EA/HCl (3 mL) and EA (3 mL) was stirred at RT for 2 h After concentration, the residue was purified by prep-HPLC (0.05% FA in water/ACN) to give title product (39 mg, Y: 38%) as a yellow solid. ESI-MS (M+H)⁺: 383.3. ¹H NMR (400 MHZ, MeOD-d₄)δ 9.62 (s, 1H), 9.13 (s. 1H), 8.85 (s, 1H), 8.20 (s, 1H), 4.33-4.16 (m, 4H), 3.54-3.46 (m, 2H), 3.27-3.15 (m, 2H), 2.63 (s, 3H), 2.37-2.24 (m, 2H), 1.95-1.80 (m, 2H).

### Example 52. 4-ethoxy-2-(ethyl(piperidin-4-yl) amino)-N-(2-methyl-2H-indazol-5-yl)pyrimidine-5-carboxamide formate (Compound 272)

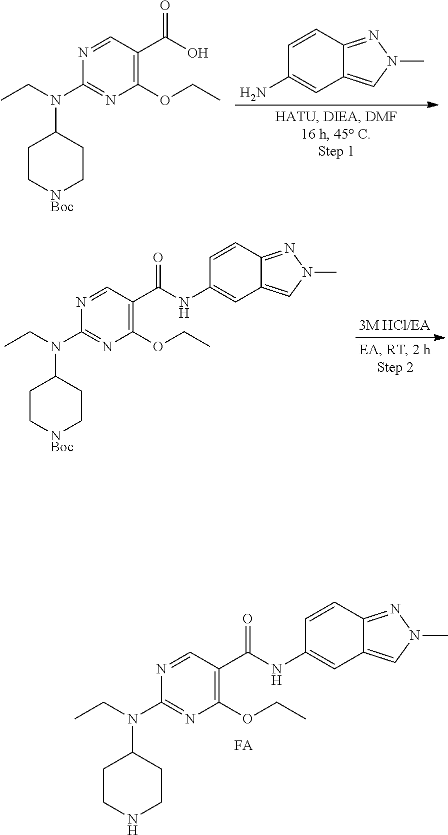

### Step 1: Preparation of tert-butyl 4-((4-ethoxy-5-((2-methyl-2H-indazol-5-yl) carbamoyl)pyrimidin-2-yl) (ethyl) amino) piperidine-1-carboxylate

To a mixture of 2-((1-(tert-butoxycarbonyl) piperidin-4-yl) (ethyl) amino)-4-ethoxypyrimidine-5-carboxylic acid (100 mg, 0.25 mmol) in DMF (5 mL) were added HATU (114 mg, 0.30 mmol), DIEA (129 mg, 1.01 mmol) and 2-methyl-2H-indazol-5-amine (44 mg, 0.30 mmol). The mixture was stirred at 45° C. for 16 h. LCMS showed the starting material was consumed completely. The mixture was diluted with water (10 mL), extracted with EA (20 mL×3). The organic layer was washed with brine, dried over Na₂SO₄ and concentrated in vacuo to give title product (100 mg, 75.19%) as a yellow solid. ESI-MS (M+H)⁺: 524.3.

### Step 2: Preparation of 4-ethoxy-2-(ethyl(piperidin-4-yl) amino)-N-(2-methyl-2H-indazol-5-yl)pyrimidine-5-carboxamide formate

To a mixture of tert-butyl 4-((4-ethoxy-5-((2-methyl-2H-indazol-5-yl) carbamoyl)pyrimidin-2-yl) (ethyl) amino) piperidine-1-carboxylate (100 mg, 0.20 mmol) in EA (3 mL) was added 3 M HCVEA (3 mL). The mixture was stirred at rt for 2 h. LCMS showed the starting material was consumed completely. The mixture was concentrated in vacuo. The crude was purified by prep-HPLC (0.05% FA in water/CH₃CN) to give title product (40 mg, Y: 49.38%) as a yellow solid. ESI-MS (M+H⁺): 424.2. ¹H NMR (400 MHZ, MeOD-d₄)δ 8.83 (s, 1H), 8.45 (s, 1H), 8.20 (s, 1H), 8.16 (s, 1H), 7.59 (d, J=9.2 Hz, 1H), 7.29 (d, J=9.2 Hz, 1H), 4.66 (q, J=6.9 Hz, 2H), 4.20 (s, 3H), 3.72-3.45 (m, 5H), 3.16 (t, J=14.0 Hz, 2H), 2.22-2.00 (m, 4H), 1.57 (t, J=7.1 Hz, 3H), 1.29 (t, J=6.6 Hz, 3H).

### Example 53. 4-ethoxy-N-(7-methoxy-2-methyl-2H-indazol-5-yl)-2-(piperazin-1-yl)pyrimidine-5-carboxamide formate (Compound 273)

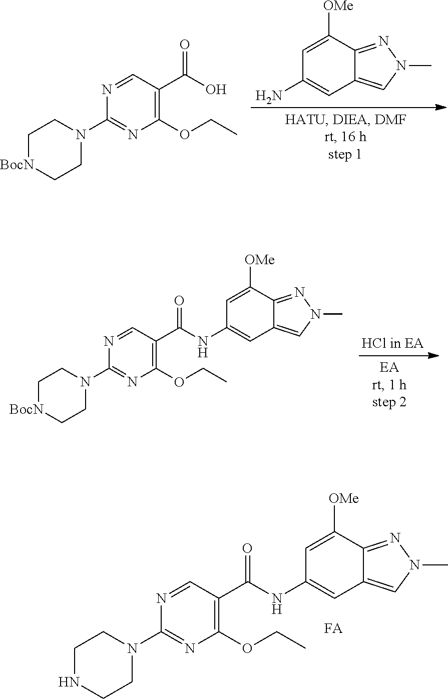

### Step 1: Preparation of tert-butyl 4-(4-ethoxy-5-((7-methoxy-2-methyl-2H-indazol-5-yl) carbamoyl)pyrimidin-2-yl) piperazine-1-carboxylate

To a mixture of 2-(4-(tert-butoxycarbonyl) piperazin-1-yl)-4-ethoxypyrimidine-5-carboxylic acid (100 mg, 0.28 mmol) in DMF (5 mL) were added 7-methoxy-2-methyl-2H-indazol-S-amine (50 mg, 0.28 mmol), HATU (162 mg, 0.42 mmol) and DIEA (110 mg, 0.85 mmol). The mixture was stirred at rt for 16 h. The mixture was diluted with (5 mL) and the precipitate was filtered and dried in vacuo to give crude title product (70 mg, Y: 48.2%) as a yellow solid. ESI-MS (M+H)⁺: 512.2. ¹H NMR (400 MHz, DMSO-d₆) δ 9.53 (s, 1H), 8.66 (s, 1H), 8.21 (s, 1H), 7.71 (s, 1H), 6.79 (s, 1H), 4.54 (q. J=7.0 Hz, 2H), 4.11 (s, 3H), 3.91 (s, 3H), 3.85-3.79 (m, 4H), 3.45-3.41 (m, 4H), 1.49-1.42 (m, 12H).

### Step 2: Preparation of 4-ethoxy-N-(7-methoxy-2-methyl-2H-indazol-5-yl)-2-(piperazin-1-yl)pyrimidine-5-carboxamide formate

A mixture of tert-butyl 4-(4-ethoxy-5-((7-methoxy-2-methyl-2H-indazol-5-yl) carbamoyl)pyrimidin-2-yl) piperazine-1-carboxylate (70 mg, 0.14 mmol) in 3 M HCV/EA (S mL) was stirred at rt for 1 h. The mixture was diluted with H₂O (5 ml), extracted with EA (10 mL×3). The aqueous layer was concentrated in vacuo and the residue was purified by prep-HPLC (0.05% FA in water/ACN) to give title product (33 mg, Y: 52.7%) as a yellow solid. ESI-MS (M+H⁺): 412.0. ¹H NMR (400 MHZ, MeOD-d₄)δ 8.85 (s, 1H), 8.34 (s, 1H), 8.11 (s, 1H), 7.64 (s, 1H), 6.82 (s, 1H), 4.65 (q, J=7.0 Hz, 2H), 4.21-4.14 (m, 8H), 4.01 (s, 3H), 1.59 (t, J=7.0 Hz, 3H).

### Example 54. 4-ethoxy-2-(3-(methylamino) pyrrolidin-1-yl)-N-(2-methylimidazo[1,2-a]pyridin-6-yl)pyrimidine-5-carboxamide (Example 274)

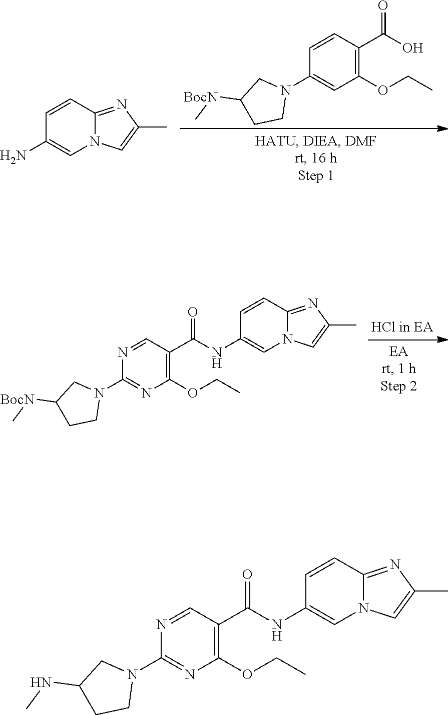

### Step 1: Preparation of tert-butyl(1-(4-ethoxy-5-((2-methylimidazo[1,2-a]pyridin-6-yl) carbamoyl)pyrimidin-2-yl) pyrrolidin-3-yl) (methyl) carbamate

To a mixture of 2-(3-((tert-butoxycarbonyl) (methyl) amino) pyrrolidin-1-yl)-4-ethoxypyrimidine-5-carboxylic acid (100 mg, 0.27 mmol) in DMF (5 mL) were added HATU (156 mg, 0.41 mmol), DIEA (106 mg, 0.82 mmol) and the mixture was stirred at rt for 1 h. 2-meth ylimidazo[1,2-a]pyridin-6-amine (140 mg, 0.27 mmol) was added. The mixture was stirred at rt for 15 h. The reaction was diluted with H₂O (10 mL), extracted with DCM (S mLx3). The organic phase was concentrated to give title product (200 mg, crude) as a black solid. ESI-MS (M+H)⁺: 496.1.

### Step 2: Preparation of 4-ethoxy-2-(3-(methylamino) pyrrolidin-1-yl)-N-(2-methylimidazo[1,2-a]pyridin-6-yl)pyrimidine-5-carboxamide

A mixture of tert-butyl(1-(4-ethoxy-5-((2-methylimidazo[1,2-a]pyridin-6-yl) carbamoyl)pyrimidin-2-yl) pyrrolidin-3-yl) (methyl) carbamate (150 mg, 0.30 mmol) in 3M HCl/EA (6 mL) was stirred at rt for 1 h. The mixture was concentrated in vacuo, the residue was purified by prep-HPLC (0.05% FA in water/ACN) to give title product (30 mg, Y: 25%) as a yellow solid. ESI-MS (M+H⁺): 396.0. ¹H NMR (400 MHz, DMSO-d₆) δ 9.72 (s, 1H), 9.31 (s, 1H), 8.67 (s, 1H), 7.86 (s, 1H), 7.57 (d, J=9.4 Hz, 1H), 7.38 (d, J=9.6 Hz, 1H), 4.56 (q, J=7.0 Hz, 2H), 3.88 (t, J=7.2 Hz, 2H), 3.80-3.71 (m, 3H), 3.68-3.61 (m, 1H), 2.66 (s, 3H), 2.36 (s, 3H), 2.18 (d, J=10.6 Hz, 1H), 1.45 (t, J=7.0 Hz, 3H).

### Example 55. 4-ethoxy-N-(7-fluoro-2-methyl-2H-indazol-5-yl)-2-(methyl(piperidin-4-yl) amino) pyrimidine-5-carboxamide formate (Compound 275)

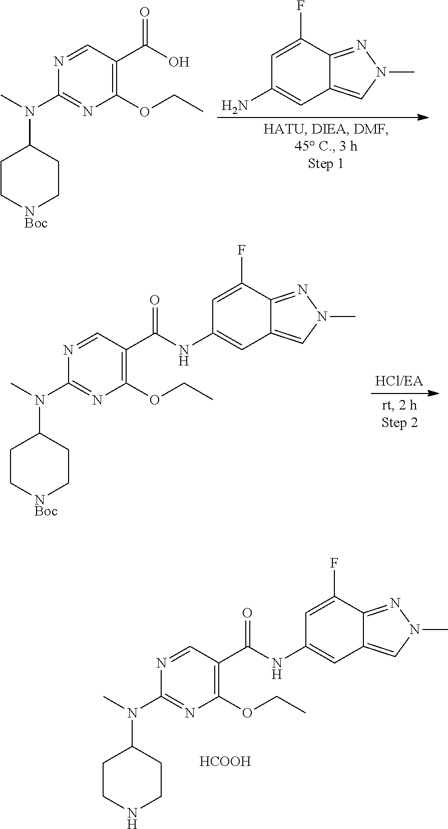

### Step 1: Preparation of tert-butyl 4-((4-ethoxy-5-((7-fluoro˜2-methyl-2H-indazol-5-yl) carbamoyl)pyrimidin-2-yl) (methyl) amino) piperidine-1-carboxylate

To a mixture of 2-((1-(tert-butoxycarbonyl) piperidin-4-yl) (methyl) amino)-4-ethoxypyrimidine-5-carboxylic acid (100 mg, 0.26 mmol) in DMF (3 mL) were added HATU (120 mg, 0.32 mmol), DIEA (136 mg, 1.05 mmol) and 7-fluoro-2-methyl-2H-indazol-S-amine (65 mg, 0.39 mmol). The mixture was stirred at 45° C. for 3 h. The reaction was diluted with H₂O (10 mL), extracted with EA (30 mL). The organic phase was concentrated to give title product (100 mg, 72%) as a yellow solid. ESI-MS (M+H)⁺: 528.5.

### Step 2: Preparation of 4-ethoxy-N-(7-fluoro-2-methyl-2H-indazol-5-yl)-2-(methyl(piperidin-4-yl) amino) pyrimidine-5-carboxamide formate

To a mixture of tert-butyl 4-((4-ethoxy-5-((7-fluoro-2-methyl-2H-indazol-5-yl) carbamoyl)pyrimidin-2-yl) (methyl) amino) piperidine-1-carboxylate (100 mg, 0.19 mmol) in EA (3 mL) was added 3 M HCl/EA (3 mL). The mixture was stirred at rt for 2 h. The mixture was concentrated in vacuo, the residue was purified by prep-HPLC (0.05% FA in water/ACN) to give title product (56 mg, Y: 62%) as a yellow solid. ESI-MS (M+H)⁺: 428.2. ¹H NMR (400 MHZ, DMSO-d₆)δ 9.60 (s, 1H), 8.64 (s, 1H), 8.43-8.31 (m, 2H), 7.97 (s, 1H), 7.29 (d, J=13.3 Hz, 1H), 4.99-4.66 (m, 1H), 4.55 (q, J=7.0 Hz, 2H), 4.17 (s, 3H), 3.23-3.17 (m, 2H), 3.05 (s, 3H), 2.84-2.75 (m, 2H), 1.89-1.78 (m, 2H), 1.73-1.63 (m, 2H), 1.45 (t, J=7.0 Hz, 3H).

### Example 56. 4-ethoxy-N-(2-methyl-2H-pyrazolo[3,4-b]pyridin-5-yl)-2-(3-(methylamino) pyrrolidin-1-yl)pyrimidine-5-carboxamide formate (Compound 276)

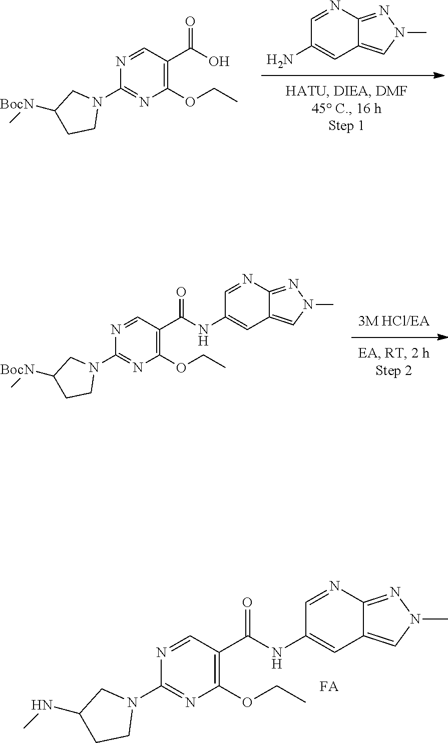

### Step 1: Preparation of ter-butyl(1-(4-ethoxy-5-((2-methyl-2H-pyrazolo[3,4-b]pyridin-5-yl) carbamoyl)pyrimidin-2-yl) pyrrolidin-3-yl) (methyl) carbamate

To a mixture of 2-(3-((tert-butoxycarbonyl) (methyl) amino) pyrrolidin-1-yl)-4-ethoxypyrimidine-5-carboxylic acid (100 mg, 0.25 mmol) in DMF (5 mL) were added HATU (114 mg, 0.30 mmol), DIEA (129 mg, 1.00 mmol) and 2-methyl-2H-pyrazolo[3,4-b]pyridin-S-amine (44 mg, 0.30 mmol). The mixture was stirred at 45° C. for 16 h. The mixture was diluted with water (10 mL) and extracted with EA (20 mL×3). The organic layer was wasbed with brine, dried over Na₂SO₄ and concentrated in vacuo to give title product (100 mg, 73.53%) as a yellow solid. ESI-MS (M+H⁺): 497.2.

### Step 2: Preparation of 4-ethoxy-N-(2-methyl-2H-pyrazolo[3,4-b]pyridin-5-yl)-2-(3-(methylamino) pyrrolidin-1-yl)pyrimidine-5-carboxamide formate

To a mixture of tert-butyl(1-(4-ethoxy-5-((2-methyl-2H-pyrazolo[3,4-b]pyridin-5-yl) carbamoyl)pyrimidin-2-yl) pyrrolidin-3-yl) (methyl) carbamate (100 mg, 0.20 mmol) in EA (3 mL) was added 3 M HCl/EA (3 mL). The mixture was stirred at rt for 2 h. The mixture was concentrated in vacuo. The crude was purified by prep-HPLC (0.05% FA in water/CH₃CN) to give title product (64 mg, Y: 80.12%) as a yellow solid. ESI-MS (M+H⁺): 397.2. ¹H NMR (400 MHZ, MeOD-d₄)δ 8.82 (s, 1H), 8.65 (s, 1H), 8.63 (s, 1H), 8.48 (s, 1H), 8.28 (s, 1H), 4.73-4.66 (m, 2H), 4.27 (s, 3H), 4.02-3.97 (m, 1H), 3.90-3.74 (m, 4H), 2.77 (s, 3H), 2.54-2.44 (m, 1H), 2.28-2.19 (m, 1H), 1.58 (t, J=7.1 Hz, 3H).

### Example 57. (S)-4-ethoxy-N-(7-fluoro-2-methyl-2H-indazol-5-yl)-2-(3-(methylamino) pyrrolidin-1-yl)pyrimidine-5-carboxamide 4-methylbenzenesulfinate (Compound 277)

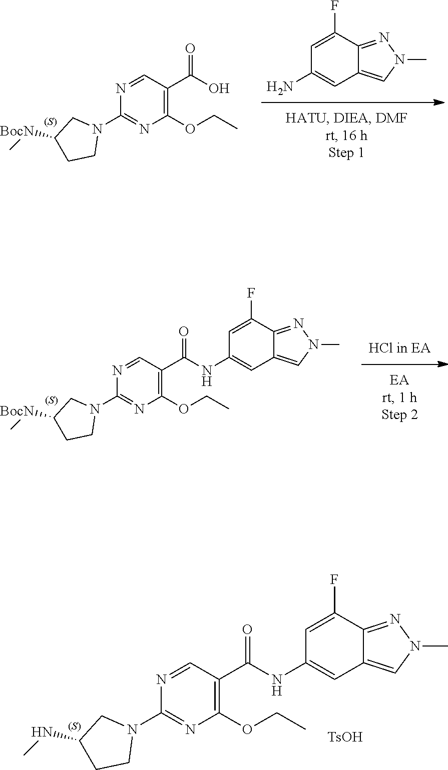

### Step 1: Preparation of tert-butyl(S)-(1-(4-ethoxy-5-(7-fluoro-2-methyl-2H-indazol-5-yl) carbamoyl)pyrimidin-2-yl) pyrrolidin-3-yl) (methyl) carbamate

To a stirred solution of(S)-2-(3-((tert-butoxycarbonyl) (methyl) amino) pyrrolidin-1-yl)-4-ethoxypyrimidine-5-carboxylic acid (300 mg,0.82 mmol), 7-fluoro-2-methyl-2H-indazol-5-aminet (135 mg, 0.82 mmol) and DIEA (528 mg, 4.10 mmol) in DMF (10 mL) was added HATU (468 mg, 1.23 mmol) at rt. The mixture was stirred for 2 h. After diluting with water, the solid was collected by filtration and washed with H₂O and dried under vacuum to give title product (400 mg, Y: 95.2%) as a yellow solid. ESI-MS (M+H)⁺: 514.2.

### Step 2: Preparation of(S)-4-ethoxy-N-(7-fluoro-2-methyl-2H-indazol-5-yl)-2-(3-(methylamino) pyrrolidin-1-yl)pyrimidine-5-carboxamide 4-methylbenzenesulfinate

To a solution of the tert-butyl(S)-(1-(4-ethoxy-5-((7-fluoro-2-methyl-2H-indazol-5-yl) carbamoyl)pyrimidin-2-yl) pyrrolidin-3-yl) (methyl) carbamate (300 mg, 0.585 mmol) in EtOAc (3 mL) was added HCl-EA (3M, 6 mL), the mixture was stirred at rt for 1 h. After concentration, the crude was purified by prep-HPLC (0.1% FA in water/CH&CN) to give title product (220 mg, yield: 91%) as a white solid. ESI-MS (M+H)⁺: 414.2. ¹H NMR (400 MHZ, DMSO-d₆) δ 9.61 (s, 1H), 8.67 (s, 1H), 8.41 (d, J=2.7 Hz, 1H), 8.24 (br, 1H), 7.97 (s, 1H), 7.47 (d, J=8.0 Hz, 2H), 7.30 (d, J=13.2 Hz, 1H), 7.11 (d, J=7.9 Hz, 2H), 4.56 (q, J=7.0 Hz, 2H), 4.18 (s, 3H), 3.87-3.61 (m, 5H), 2.61 (s, 3H), 2.36-2.30 (m, 1H), 2.28 (s, 3H), 2.16-2.08 (m, 1H), 1.46 (t, J=7.0 Hz, 3H).

### Example 58. 4-ethoxy-N-(7-methoxy-2-methyl-2H-indazol-5-yl)-2-(3-(methylamino) pyrrolidin-1-yl)pyrimidine-5-carboxamide formate (Compound 278)

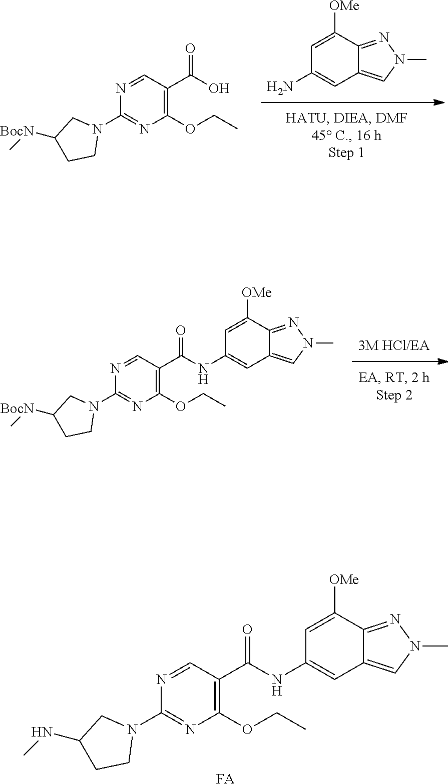

### Step 1: Preparation of tert-butyl(1-(4-ethoxy-5-((7-methoxy-2-methyl-2H-indazol-5-yl) carbamoyl)pyrimidin-2-yl) pyrrolidin-3-yl) (methyl) carbamate

To a mixture of 2-(3-((tert-butoxycarbonyl) (methyl) amino) pyrrolidin-1-yl)-4-ethoxypyrimidine-5-carboxylic acid (100 mg, 0.25 mmol) in DMF (5 mL) were added HATU (114 mg, 0.30 mmol), DIEA (129 mg, 1.00 mmol) and 7-methoxy-2-methyl-2H-indazol-5-amine (53 mg, 0.30 mmol). The mixture was stirred at 45° C. for 16 h. The mixture was diluted with water (10 mL), extracted with EA (20 mL×3). The organic layer was washed with brine, dried over Na₂SO₄ and concentrated in vacuo to give title product (120 mg, 83.33%) as a yellow solid. ESI-MS (M+H⁺): 526.1

### Step 2: Preparation of 4-ethoxy-N-(7-methoxy-2-methyl-2H-indazol-5-yl)-2-(3-(methylamino) pyrrolidin-1-yl)pyrimidine-5-carboxamide formate

To a mixture of tert-butyl(1-(4-ethoxy-5-((7-methoxy-2-methyl-2H-indazol-5-yl) carbamoyl)pyrimidin-2-yl) pyrrolidin-3-yl) (methyl) carbamate (120 mg, 0.26 mmol) in EA (3 mL) was added 3 M HCl/EA (3 mL). The mixture was stirred at rt for 2 h. The mixture was concentrated in vacuo. The crude was purified by prep-HPLC (0.05% FA in water/CH₃CN) to give title product (56 mg, Y: 57.73%) as a yellow solid. ESI-MS (M+H⁺): 426.1. ¹H NMR (400 MHZ, MeOD-d₄) δ 8.82 (s, 1H), 8.44 (s, 1H), 8.12 (s, 1H), 7.64 (s, 1H), 6.80 (s, 1H), 4.72-4.61 (m, 2H), 4.19 (s, 3H), 4.01 (s, 3H), 4.00-3.95 (m, 1H), 3.94-3.71 (m, 4H), 2.79 (s, 3H), 2.56-2.45 (m, 1H), 2.26-2.24 (m, 1H), 1.60 (t, J=7.0 Hz, 3H).

### Example 59. (S)-4-ethoxy-N-(7-fluoro-2-methyl-2H-indazol-5-yl)-2-(3-methylpiperazin-1-yl)pyrimidine-5-carboxamide formate (Compound 279)

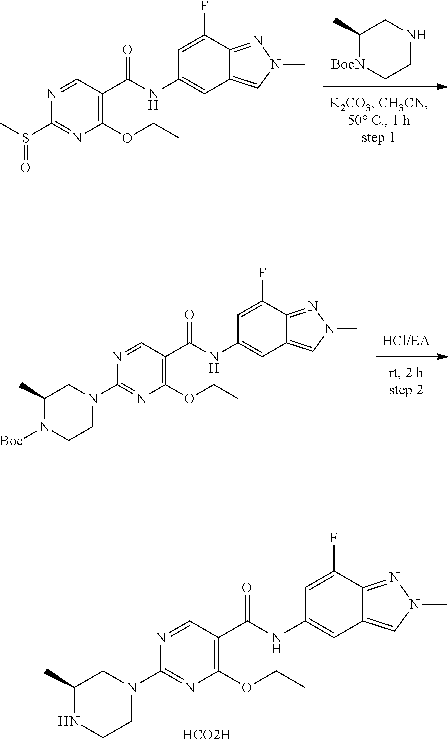

### Step 1: Preparation of tert-butyl(S)-4-(4-ethoxy-5-((7-fluoro-2-methyl-2H-indazol-5-yl) carbamoyl)pyrimidin-2-yl)-2-methylpiperazine-1-carboxylate

To a mixture of 4-ethoxy-N-(7-fluoro-2-methyl-2H-indazol-5-yl)-2-(methylsulfinyl)pyrimidine-5-carboxamide (100 mg, 0.26 mmol) in CH₃CN (3 mL) were added tert-butyl(S)-2-methylpiperazine-1-carboxylate (80 mg, 0.39 mmol) and K₂CO₃ (73 mg, 0.53 mmol). The mixture was stirred at 50° C. for 1 h. The mixture was diluted with H₂O (5 mL) and extracted with EA (10 mL×3). The organic phase was concentrated in vacuo to give title product (100 mg, Y: 74%) as a yellow solid. ESI-MS (M+H)⁺: 514.3.

### Step 2: Preparation of(S)-4-ethoxy-N-(7-fluoro-2-methyl-2H-indazol-5-yl)-2-(3-methylpiperazin-1-yl)pyrimidine-5-carboxamide formate

A mixture of tert-butyl(S)-4-(4-ethoxy-5-((7-fluoro-2-methyl-2H-indazol-5-yl) carbamoyl)pyrimidin-2-yl)-2-methylpiperazine-1-carboxylate (100 mg, 0.19 mmol) in 3M HCl/EA (3 mL) was stirred at rt for 2 h. The mixture was diluted H₂O (5 mL) and extracted with EA (10 mL×3). The aqueous layer was concentrated in vacuo and the residue was purified by prep-HPLC (0.05% FA in water/ACN) to give title product (40 mg, Y: 45%) as a yellow solid. ESI-MS (M+H)⁺: 414.0. ¹H NMR (400 MHZ, DMSO-d₆)δ 9.60 (s, 1H), 8.63 (s, 1H), 8.41 (s, 1H), 8.20 (s, 1H), 7.97 (s, 1H), 7.31-7.26 (m, 1H), 4.60-4.48 (m, 4H), 4.17 (s, 3H), 3.04-2.95 (m, 2H), 2.77-2.62 (m, 3H), 1.44 (t, J=7.0 Hz, 3H), 1.07 (d, J=6.0 Hz, 3H).

### Example 60. 2-((3R,5S)-3,5-dimethylpiperazin-1-yl)-4-ethoxy-N-(7-fluoro-2-methyl-2H-indazol-5-yl)pyrimidine-5-carboxamide 4-methylbenzenesulfonate (Compound 280)

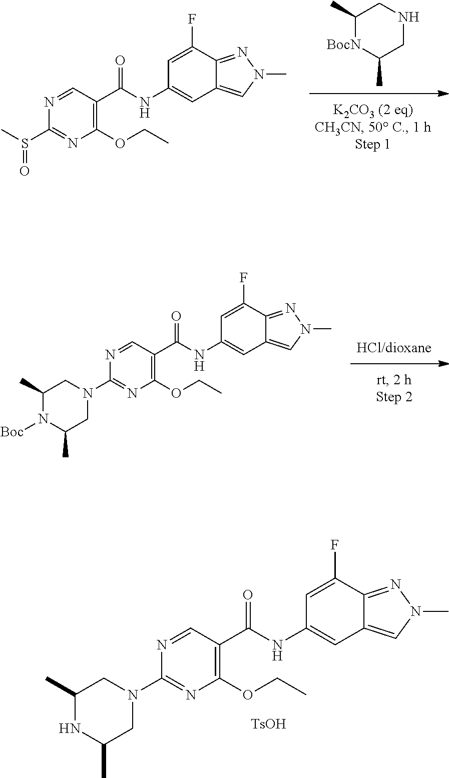

### Step 1: Preparation of tert-butyl(2R.6S)-4-(4-ethoxy-5-((7-fluoro-2-methyl-2H-indazol-5-yl) carbamoyl)pyrimidin-2-yl)-2,6-dimethylpiperazine-1-carboxylate

To a mixture of 4-ethoxy-N-(7-fluoro-2-methyl-2H-indazol-5-yl)-2-(methylsulfinyl)pyrimidine-5-carboxamide (500 mg, 1.33 mmol) and tert-butyl piperazine-1-carboxylate (285 mg, 1.33 mmol) in ACN (10 mL) was added K₂CO₃ (366 mg, 2.65 mmol). The mixture was stirred at 50° C. for 1 h. The reaction mixture was diluted with H₂O (10 mL), extracted with DCM (15 mL×3). The organic layer was washed with brine, dried over Na₂SO₄ and evaporated to give crude. The crude was triturated with EA (5 mL) to give title product (220 mg, yield 31.5%) as a brown solid. ESI-MS (M+H)⁺: 528.2.

### Step 2: Preparation of 2-((3R,5S)-3,5-dimethylpiperazin-1-yl)-4-ethoxy-N-(7-fluoro-2-methyl-2H-indazol-5-yl)pyrimidine-5-carboxamide+-methylbenzenesulfonate

A mixture of tert-butyl(2R,6S)-4-(4-ethoxy-5-((7-fluoro-2-methyl-2H-indazol-5-yl) carbamoyl)pyrimidin-2-yl)-2,6-dimethylpiperazine-1-carboxylate (220 mg, 0.417 mmol) in 3M HCl/EA (5 mL) was stirred at rt for 2 h. The mixture was concentrated in vacuo and the residue was purified by reversed phase column (0.05% NH₃H₂O in water/ACN) and concentrated in vacuo. The solid was dissolved in the solution of TsOH (1 eq) in water (3 mL) and lyophilized to give title product (120 mg, Y: 48%) as a white solid. ESI-MS (M+H)⁺: 428.2. ¹H NMR (400 MHz, DMSO-d₆) δ 9.67 (s, 1H), 8.67 (s, 1H), 8.42 (d, J=2.8 Hz, 1H), 7.98 (s, 1H), 7.48 (d, J=8.1 Hz, 2H), 7.29 (dd, J=13.3, 1.3 Hz, 1H), 7.11 (d, J=7.9 Hz, 2H), 4.82 (d, J=12.4 Hz, 2H), 4.55 (q, J=7.0 Hz, 2H), 4.18 (s, 3H), 3.30-3.25 (m, 2H), 2.89 (t, J=12.5 Hz, 2H), 2.29 (s, 3H), 1.45 (t, J=7.0 Hz, 3H), 1.27 (d, J=6.4 Hz, 6H).

### Example 61. N-(7-fluoro-2-methyl-2H-indazol-5-yl)-4-methoxy-2-(methyl(piperidin-4-yl) amino) pyrimidine-5-carboxamide formate (Compound 281)

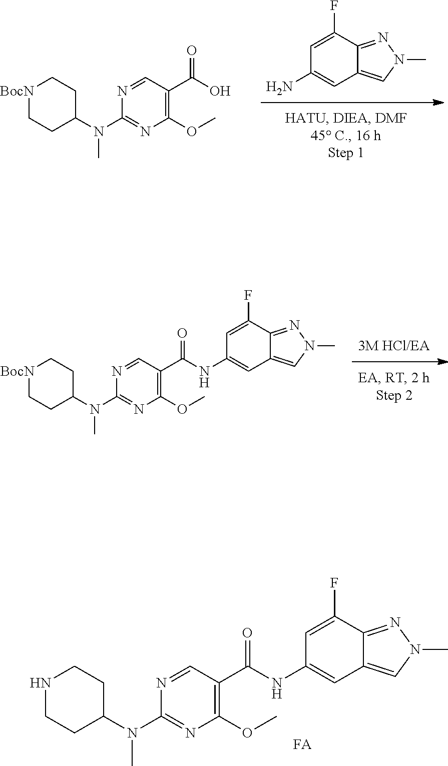

### Step 1: Preparation of tert-butyl 4-((5-((7-fluoro-2-methyl-2H-indazol-5-yl) carbamoyl)-4-methoxypyrimidin-2-yl) (methyl) amino) piperidine-1-carboxylate

To a mixture of 2-((1-(tert-butoxycarbonyl) piperidin-4-yl) (methyl) amino)-4-methoxypyrimidine-5-carboxylic acid (100 mg, 0.25 mmol) in DMF (5 mL) were added HATU (125 mg, 0.30 mmol), DIEA (141 mg, 1.00 mmol) and 7-fluoro-2-methyl-2H-indazol-5-amine (54 mg, 0.30 mmol). The mixture was stirred at 45° C. for 16 h. The mixture was diluted with water (10 mL) and extracted with EA (20 mL×3). The organic layer was washed with brine, dried over Na₂SO₄ and concentrated in vacuo to give title product (90 mg, 64.29%) as a yellow solid. ESI-MS (M+H⁺): 514.3.

### Step 2: Preparation of N-(7-fluoro-2-methyl-2H-indazol-5-yl)-4-methoxy-2-(methyl(piperidin-4-yl) amino) pyrimidine-5-carboxamide formate

To a mixture of tert-butyl 4-((5-((7-fluoro-2-methyl-2H-indazol-5-yl) carbamoyl)-4-methoxypyrimidin-2-yl) (methyl) amino) piperidine-1-carboxylate (90 mg, 0.18 mmol) in EA (3 mL) was added 3 M HCl/EA (3 mL). The mixture was stirred at rt for 2 h. The mixture was concentrated in vacuo. The crude was purified by prep-HPLC (0.05% FA in water/CH 3CN) to give title product (44 mg, Y: 60.70%) as a yellow solid. ESI-MS (M+H⁺): 414.1. ¹H NMR (400 MHZ, MeOD-d₄) δ 8.82 (s, 1H), 8.53 (s, 1H), 8.26 (s, 1H), 7.91 (s, 1H), 7.28 (d, J=12.7 Hz, 1H), 5.07-4.94 (m, 1H), 4.24 (s, 3H), 4.20 (s, 3H), 3.58-3.54 (m, 2H), 3.25-3.16 (m, 5H), 2.18-1.99 (m, 4H).

### Example 62. (R)-4-ethoxy-N-(7-fluoro-2-methyl-2H-indazol-5-yl)-2-(3-(methylamino) pyrrolidin-1-yl)pyrimidine-5-carboxamide formate (Compound 282)

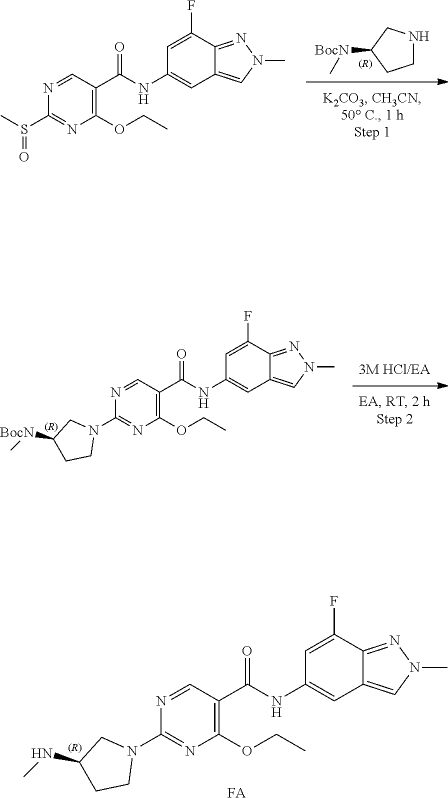

### Step 1: Preparation of ter-butyl(R)-(1-(4-ethoxy-5-(7-fluoro-2-methyl-2H-indazol-5-yl) carbamoyl)pyrimidin-2-yl) pyrrolidin-3-vl) (methyl) carbamate

To a mixture of 4-ethoxy-N-(7-fluoro-2-methyl-2H-indazol-5-yl)-2-(methylsulfinyl)pyrimidine-5-carboxamide (500 mg, 1.33 mmol) in CH₃CN (10 mL) were added K₂CO₃ (367 mg, 2.66 mmol) and tert-butyl(R)-methyl( pyrrolidin-3-yl) carbamate (266 mg, 1.33 mmol). The mixture was stirred at 50° C. for 1 h. The mixture was diluted with water (10 mL), extracted with EA (20 mL×3). The organic layer was washed with brine, dried over Na₂SO₄ and concentrated in vacuo to give title product (600 mg, 87.98%) as a yellow solid. ESI-MS (M+H⁺): 514.3.

### Step 2: Preparation of (R)-4-ethoxy-N-(7-fluoro-2-methyl-2H-indazol-5-yl)-2-(3-(methylamino) pyrrolidin-1-yl)pyrimidine-5-carboxamide formate

To a mixture of tert-butyl(R)-(1-(4-ethoxy-5-((7-fluoro-2-methyl-2H-indazol-5-yl) carbamoyl)pyrimidin-2-yl) pyrrolidin-3-yl) (methyl) carbamate (600 mg, 1.17 mmol) in EA (3 mL) was added 3 M HCl/EA (5 mL). The mixture was stirred at rt for 2 h. The mixture was concentrated in vacuo. The crude was purified by prep-HPLC (0.05% FA in water/CH₃CN) to give title product (395 mg, Y: 73.5%) as a yellow solid. ESI-MS (M+H⁺): 414.2. ¹H NMR (400 MHz, MeOD-d₄) δ 8.79 (s, 1H), 8.44 (s, 1H), 8.24 (d, J=2.7 Hz, 1H), 7.96-7.84 (m, 1H), 7.28-7.18 (m, 1H), 4.72-4.60 (m, 2H), 4.22 (s, 3H), 4.01-3.96 (m, 1H), 3.90-3.72 (m, 4H), 2.74 (s, 3H), 2.54-2.41 (m, 1H), 2.29-2.13 (m, 1H), 1.56 (t, J=7.1 Hz, 3H).

### Example 63. 4-ethoxy-N-(8-methoxy-2-methylimidazo[1,2-a]pyridin-6-yl)-2-(3-(methylamino) pyrrolidin-1-yl)pyrimidine-5-carboxamide formate (Compound 283)

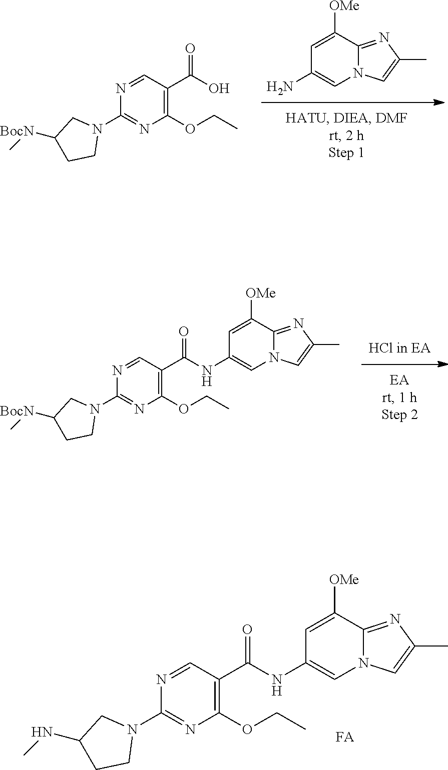

### Step 1: Preparation of tert-butyl(1-(4-ethoxy-5-((8-methoxy-2-methylimidazo[1,2-a]pyridin-6-yl) carbamoyl)pyrimidin-2-yl) pyrrolidin-3-yl) (methyl) carbamate

To a mixture of 2-(3-((tert-butoxycarbonyl) (methyl) amino) pyrrolidin-1-yl)-4-ethoxypyrimidine-5-carboxylic acid (100 mg, 0.27 mmol) in DMF (5 mL) were added HATU (155.7 mg, 0.41 mmol) and DIEA (176 mg, 1.37 mmol) and the mixture was stirred at rt for 0.5 h. Then 8-methoxy-2-methylimidazo[1,2-a]pyridin-6-amine (48 mg, 0.27 mmol) was added and the mixture was stirred at rt for 2 h. The mixture was diluted with H₂O (15 mL) and stirred for 10 min. The precipitate was filtered, washed with water and dried in vacuo to give title product (80 mg, Y: 55.8%) as a yellow solid. ESI-MS (M+H)⁺: 526.1.

### Step 2: Preparation of 4-ethoxy-N-(8-methoxy-2-methylimidazo[1,2-a]pyridin-6-yl)-2-(3-(methylamino) pyrrolidin-1-yl)pyrimidine-5-carboxamide formate

A mixture of tert-butyl(1-(4-ethoxy-5-((8-methoxy-2-methylimidazo[1,2-a]pyridin-6-yl) carbamoyl)pyrimidin-2-yl) pyrrolidin-3-yl) (methyl) carbamate (80 mg, 0.15 mmol) in 3M HCl/EA (2 mL) was stirred at rt for 1 h. The mixture was concentrated in vacuo and the residue was purified by prep-HPLC (0.05% FA in water/CH₃CN) to give title product (38.5 mg, Y: 59.4%) as an orange solid. ESI-MS (M+H⁺): 426.1. ¹H NMR (400 MHZ, MeOD-d₄)δ 8.96-8.68 (m, 2H), 8.46 (s, 1H, HCO2H), 7.61 (s, 1H), 6.76-6.33 (m, 1H), 4.76-4.46 (m, 2H), 4.02 (s, 3H), 3.96-3.72 (m, 3H), 3.60-3.47 (m, 1H), 3.45-3.36 (m, 1H), 2.90-2.70 (m, 3H), 2.55-2.46 (m, 1H), 2.43 (s, 3H), 2.28-2.17 (m, 1H), 1.58 (t, J=7.0 Hz, 3H).

### Example 64. 2-(ethyl(piperidin-4-yl) amino)-N-(7-fluoro-2-methyl-2H-indazol-5-yl)-4-methoxypyrimidine-5-carboxamide formate (Compound 284)

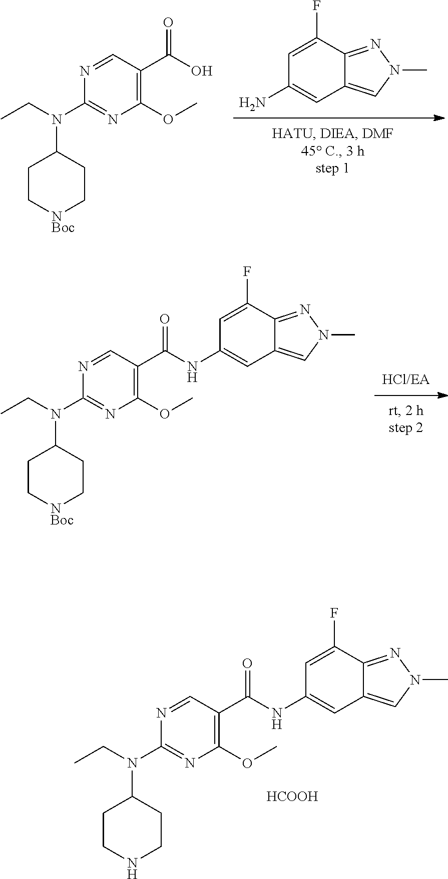

### Step 1: Preparation of tert-butyl 4-(ethyl(5-((7-fluoro-2-methyl-2H-indazol-5-yl) carbamoyl)-4-methoxypyrimidin-2-yl) amino) piperidine-1-carboxylate

To a mixture of 2-((1-(tert-butoxycarbonyl) piperidin-4-yl) (ethyl) amino)-4-methoxypyrimidine-5-carboxylic acid (100 mg, 0.26 mmol) in DMF (3 mL) were added 7-fluoro-2-methyl-2H-indazol-5-amine (65 mg, 0.39 mmol), HATU (120 mg, 0.32 mmol) and DIEA (136 mg, 1.10 mmol). The mixture was stirred at 45° C. for 3 h. The mixture was diluted with H₂O (5 mL) and stirred at rt for 10 min. The precipitate was filtered, washed with water and dried in vacuo to give crude product (100 mg, Y: 72%) as a yellow solid. ESI-MS (M+H)⁺: 528.3.

### Step 2: Preparation of 2-(ethyl(piperidin-4-yl) amino)-N-(7-fluoro-2-methyl-2H-indazol-5-yl)-4-methoxypyrimidine-5-car boxamide formate

A mixture of tert-butyl 4-(ethyl(5-((7-fluoro-2-methyl-2H-indazol-5-yl) carbamoyl)-4-methoxypyrimidin-2-yl) amino) piperidine-1-carboxylate (100 mg, 0.19 mmol) in 3M HCl/EA (3 mL) was stirred at rt for 2 h. The mixture was added diluted with water (5 mL) and extracted with EA (10 mL×3). The aqueous layer was concentrated in vacuo and the residue was purified by prep-HPLC (0.05% FA in water/ACN) to give title product (25 mg, Y: 28%) as a yellow solid. ESI-MS (M+H⁺): 428.1. ¹H NMR (400 MHZ, MeOD-d₄)δ 8.80 (s, 1H), 8.54 (s, 1H), 8.24 (s, 1H), 7.89 (s, 1H), 7.26 (d, J=12.8 Hz, 1H), 4.90-4.85 (m, 1H), 4.22 (s, 3H), 4.18 (s, 3H), 3.70-3.64 (m, 2H), 3.53-3.47 (m, 2H), 3.17-3.09 (m, 2H), 2.20-2.08 (m, 2H), 2.06-1.99 (m, 2H), 1.29 (t, J=6.5 Hz, 3H).

### Example 65. (S)-4-ethoxy-N-(2-methyl-2H-indazol-5-yl)-2-(3-(methylamino) pyrrolidin-1-yl)pyrimidine-5-carboxamide 2,2,2-trifluoroacetate (Compound 285)

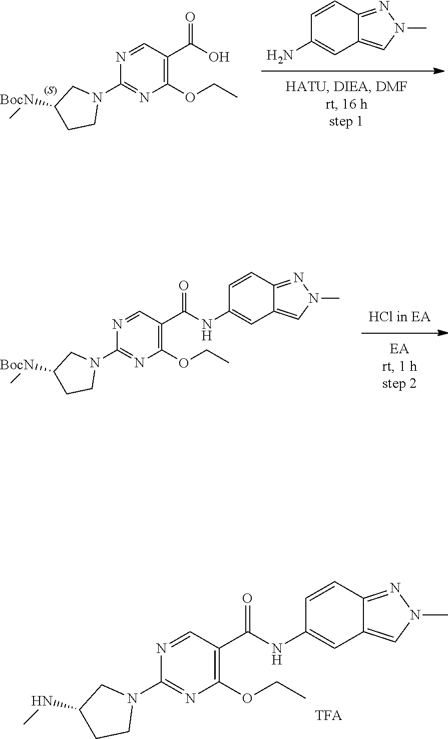

### Step 1: Preparation of tert-butyl(S)-(1-(4-ethoxy-5-((2-methyl-2H-indazol-5-yl) carbamoyl)pyrimidin-2-yl) pyrrolidin-3-yl) (methyl) carbamate

To a mixture of(S)-2-(3-((tert-butoxycarbonyl) (methyl) amino) pyrrolidin-1-yl)-4-ethoxypyrimidine-5-carboxylic acid (300 mg, 0.82 mmol) in DMF (4 mL) were added 2-methyl-2H-indazol-5-amine (180.73 mg, 1.23 mmol), HATU (467.21 mg, 1.23 mmol) and DIEA (422.95 mg, 3.27 mmol). The mixture was stirred at rt for 16 h. The mixture was diluted with water (50 mL) and stirred at rt for 10 min. The precipitate was filtered, washed with water and dried in vacuo to give crude product (350 mg, Y: 86.4%) as a yellow solid. ESI-MS (M+H)⁺: 496.2.

### Step 2: Preparation of(S)-4-ethoxy-N-(2-methyl-2H-indazol-5-yl)-2-(3-(methylamino) pyrrolidin-1-yl)pyrimidine-5-carboxamide 2,2,2-trifluoroacetate

A mixture of tert-butyl(S)-(1-(4-ethoxy-5-((2-methyl-2H-indazol-5-yl) carbamoyl)pyrimidin-2-yl) pyrrolidin-3-yl) (methyl) carbamate (350 mg, 0.71 mmol) in 3M HCl/EA (3 mL) was stirred at rt for 2 h. The mixture was diluted with water (15 mL), extracted with EA (20 mL×3). The aqueous phase was concentrated in vacuo and the crude was purified by prep-HPLC (0.05% TFA in water/ACN) to give title product (188 mg, Y: 52.2%) as a yellow solid. ESI-MS (M+H⁺): 396.1. ¹H NMR (400 MHz, DMSO-d₆) δ 9.59 (s, 1H), 8.88 (s, 2H), 8.70 (s, 1H), 8.28 (s, 1H), 8.24 (s, 1H), 7.58 (d, J=9.1 Hz, 1H), 7.29 (d, J=9.2, 1.7 Hz, 1H), 4.57 (q, J=7.0 Hz, 2H), 4.15 (s, 3H), 3.97-3.85 (m, 2H), 3.77 (t, J=7.7 Hz, 2H), 3.67-3.63 (m, 1H), 2.67 (t, J=5.2 Hz, 3H), 2.41-2.32 (m, 1H), 2.19-2.16 (m, 1H), 1.48 (t, J=7.0 Hz, 3H).

### Example 66. (R)-4-ethoxy-N-(2-methyl-2H-indazol-5-yl)-2-(3-(methylamino) pyrrolidin-1-yl)pyrimidine-5-carboxamide formate (Compound 286)

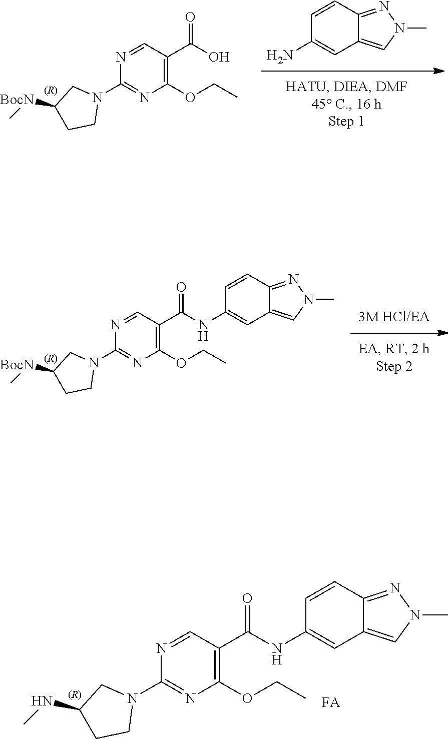

### Step 1: Preparation of ter-butyl(R)-(1-(4-ethoxy-5-(2-methyl-2H-indazol-5-yl) carbamoyl)pyrimidin-2-yl) pyrrolidin-3-yl) (methyl) carbamate

To a mixture of (R)-2-(3-((tert-butoxycarbonyl) (methyl) amino) pyrrolidin-1-yl)-4-ethoxypyrimidine-S-carboxylic acid (350 mg, 0.96 mmol) in DMF (5 mL) were added HATU (437 mg, 1.15 mmol), DIEA (495 mg, 3.84 mmol) and 2-methyl-2H-indazol-5-amine (169 mg, 1.15 mmol). The mixture was stirred at 45° C. for 16 h. The mixture was diluted with water (10 mL), extracted with EA (20 mL×3). The organic layer was washed with brine, dried over Na₂SO₄ and concentrated in vacuo to give title product (400 mg, 84.39%) as a yellow solid. ESI-MS (M+H⁺): 496.1.

### Step 2: Preparation of (R)-4-ethoxy-N-(2-methyl-2H-indazol-5-yl)-2-(3-(methylamino) pyrrolidin-1-yl)pyrimidine-5-carboxamide formate

To a mixture of tert-butyl(R)-(1-(4-ethoxy-5-((2-methyl-2H-indazol-5-yl) carbamoyl)pyrimidin-2-yl) pyrrolidin-3-yl) (methyl) carbamate (400 mg, 0.81 mmol) in EA (3 mL) was added 3 M HCl/EA (S mL). The mixture was stirred at rt for 2 h. LCMS showed the starting material was consumed completely. The mixture was concentrated in vacuo. The crude was purified by prep-HPLC (0.05% FA in water/CH₃CN) to give title product (238 mg, Y: 74.61%) as a yellow solid. ESI-MS (M+H⁺): 396.2. ¹H NMR (400 MHZ, MeOD-d₄) δ 8.83 (s, 1H), 8.38 (s, 1H), 8.20 (s, 1H), 8.16 (s, 1H), 7.59 (d, J=9.1 Hz, 1H), 7.32-7.27 (m, 1H), 4.68 (q, J=7.1 Hz, 2H), 4.20 (s, 3H), 4.04-3.97 (m, 1H), 3.93-3.74 (m, 4H), 2.77 (s, 3H), 2.56-2.44 (m, 1H), 2.32-2.19 (m, 1H), 1.57 (t, J=7.1 Hz, 3H).

### Example 67. 4-ethoxy-2-(3-(methylamino) pyrrolidin-1-yl)-N-(2-methylimidazo[1,2-a]pyrimidin-6-yl)pyrimidine-5-carboxamide fomate (Compound 287)

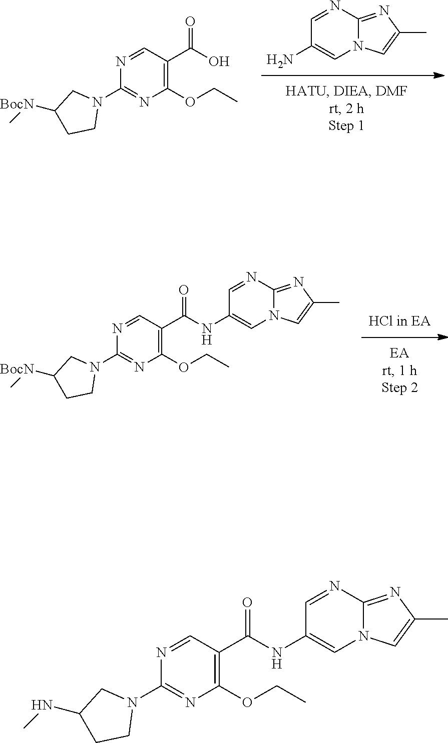

### Step 1: Preparation of tert-butyl(1-(4-ethoxy-5-((2-methylimidazo[1,2-a]pyrimidin-6-yl) carbamoyl)pyrimidin-2-yl) pyrrolidin-3-yl) (methyl) carbamate

To a stirred solution of 2-(3-((tert-butoxycarbonyl) (methyl) amino) pyrrolidin-1-yl)-4-ethoxypyrimidine-5-carboxylic acid (50 mg, 0.14 mmol), 2-methylimidazo[1,2-a]pyrimidin-6-amine (20.2 mg. 0.14 mmol) and DIEA (88 mg, 0.68 mmol) in DMF (2 mL) was added HATU (77.87 mg, 0.21 mmol) and the mixture was stirred at rt for 2 h. After diluting with water, the solid was collected by filtration and washed with H₂O to give title product (55 mg, Y: 81%) as a yellow solid. ESI-MS (M+H)⁺: 497.1.

### Step 2: Preparation of 4-ethoxy-2-(3-(methylamino) pyrrolidin-1-yl)-N-(2-methylimidazo[1,2-a]pyrimidin-6-yl)pyrimidine-5-carboxamide formate

To a solution of the tert-butyl(1-(4-ethoxy-5-((2-methylimidazo[1,2-a]pyrimidin-6-yl) carbamoyl)pyrimidin-2-yl) pyrrolidin-3-yl) (methyl) carbamate (50 mg. 0.10 mmol) in EtOAc (1 mL) was added HCl-EA (3M, 2 mL), the mixture was stirred at rt for 1 h. After concentration, the crude was purified by prep-HPLC (0.1% FA in water/CH₃CN) to give title product (15 mg, yield: 37.6%) as a white solid. ESI-MS (M+H)⁺: 397.0. ¹H NMR (400 MHZ, MeOD-d₄)δ 9.45 (d, J=2.1 Hz, 1H), 8.80 (s, 1H), 8.47 (d, J=2.4 Hz, 1H), 8.40 (s, 1H), 7.60 (s, 1H), 4.68 (q, J=6.9 Hz, 2H), 3.99-3.98 (m, 1H), 3.91-3.71 (m, 4H), 2.76 (s, 3H), 2.53-2.46 (m, 1H), 2.44 (s, 3H), 2.29-2.16 (m, 1H), 1.56 (t, J=7.0 Hz, 3H).

### Example 68. 4-ethoxy-2-(ethyl(piperidin-4-yl) amino)-N-(2-methylpyrazolo[1,5-a]pyridin-5-yl)pyrimidine-5-carboxamide formate (Compound 288)

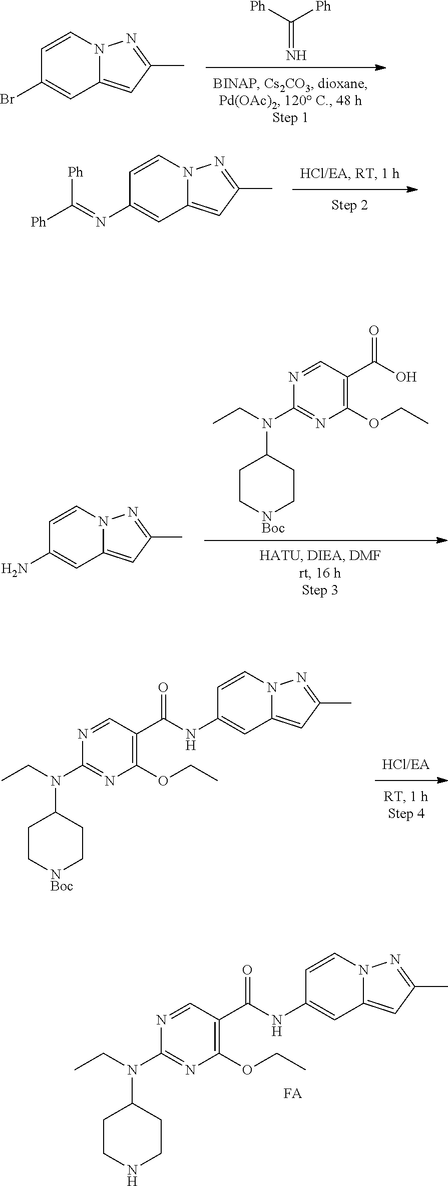

### Step 1: Preparation of N-(2-methylpyrazolo[1,5-a]pyridin-5-yl)-1,1-diphenylmethanimine

To a mixture of 5-bromo-2-methylpyrazolo[1,5-a]pyridine (1.80 g, 8.57 mmol) and diphenylmethanimine (1.71 g, 9.43 mmol) in dioxane (100 mL) were added BINAP (1.07 g, 1.71 mmol), Pd(OAc)₂ (0.21 g, 0.86 mmol) and Cs₂CO₃ (5.57 g, 17.14 mmol). The reaction solution was stirred at 120° C. for 48 h. The reaction was diluted with H₂O (50 mL), extracted with EA (50 mL×3), the organic layer was washed with brine, dried over Na₂SO₄ and evaporated to give crude. The crude was purified by reserve silica gel column chromatography (PE:EA=5:1) to give title product (1.6 g, yield: 60%) as a yellow solid. ESI-MS (M+H)⁺: 312.0.

### Step 2: Preparation of 2-methylpyrazolo[1,5-a]pyridin-5-amine

A mixture of N-(2-methylpyrazolo[1,5-a]pyridin-5-yl)-1,1-diphenylmethanimine (1.6 g, 5.14 mmol) in 3M HCl/EA (10 mL) was stirred at RT for 1 b. LCMS showed the reaction was completed. The reaction was diluted with H₂O (10 mL), extracted with EA (10 mL×2), the aqueous layer concentrated in vacuo to give title product (600 mg, Y: 94%) as a yellow solid. ESI-MS (M+H⁺): 148.2.

### Step 3: Preparation of tert-butyl 4-(4-ethoxy-5-(2-methylpyrazolo[1,5-a]pyridin-5-yl) carbamoyl)pyrimidin-2-yl) (ethyl) amino) piperidine-1-carboxylate

To a mixture of 2-((1-(tert-butoxycarbonyl) piperidin-4-yl) (ethyl) amino)-4-ethoxypyrimidine-5-carboxylic acid (134 mg, 0.34 mmol) in DMF (5 mL) were added HATU (194 mg, 0.51 mmol) and DIEA (219 mg, 1.70 mmol) and the mixture was stirred at rt for 0.5 h. Then 2-methylpyrazolo[1,5-a]pyridin-5-amine (50 mg, 0.34 mmol) was added and the mixture was stirred at rt for 16 h. The mixture was diluted with H₂O (15 mL) and the precipitate was filtered and dried in vacuo to give crude product (200 mg, crude) as a yellow solid. ESI-MS (M+H)⁺: 524.2.

### Step 4: Preparation of 4-ethoxy-2-(ethyl(piperidin-4-yl) amino)-N-(2-methylpyrazolo[1,5-a]pyridin-5-yl)pyrimidine-5-carboxamide formate

A mixture of tert-butyl 4-((4-ethoxy-5-((2-methylpyrazolo[1,5-a]pyridin-5-yl) carbamoyl)pyrimidin-2-yl) (ethyl) amino) piperidine-1-carboxylate (150 mg, 0.29 mmol) in 3M HCl/EA (2 mL) was stirred at rt for 1 h. The mixture was concentrated in vacuo, the residue was purified by prep-HPLC (0.1% FA in water/CH₃CN) to give title product (52.57 mg, Y: 43%) as a gray solid. ESI-MS (M+H⁺): 424.1. ¹H NMR (400 MHZ, MeOD-d₄) δ 8.80 (s, 1H), 8.46 (s, 1H), 8.33 (d, J=7.5 Hz, 1H), 8.07 (d, J=2.0 Hz, 1H), 6.87 (dd, J=7.5, 2.2 Hz, 1H), 6.28 (s, 1H), 4.65 (q, J=7.0 Hz, 2H), 3.69-3.62 (m, 2H), 3.56-3.49 (m, 2H), 3.20-3.11 (m, 2H), 2.42 (s, 3H), 2.37-2.07 (m, 3H), 2.06-1.92 (m, 2H), 1.57 (t, J=7.1 Hz, 3H), 1.28 (t, J=6.7 Hz, 3H).

### Example 69. (R)-4-ethoxy-2-(3-(methylamino) pyrrolidin-1-yl)-N-(2-methylimidazo[1,2-a]pyridin-6-yl)pyrimidine-5-carboxamide formate (Compound 289)

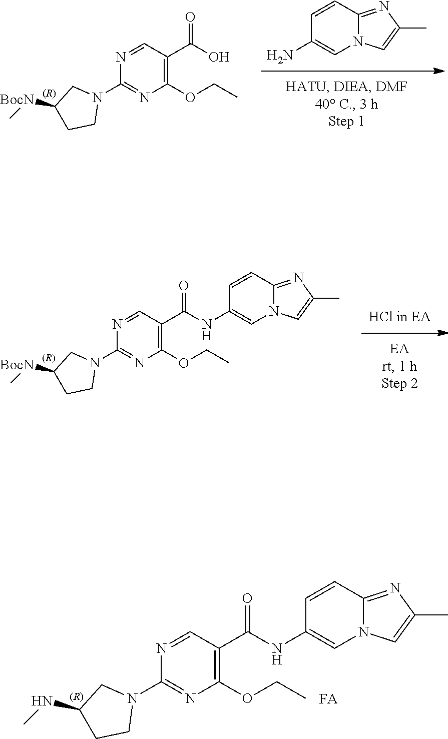

### Step 1: Preparation of tert-butyl(R)-(1-(4-ethoxy-5-((2-methylimidazo[1,2-a]pyridin-6-y) carbamoyl)pyrimidin-2-yl) pyrrolidin-3-yl) (methyl) carbamate

To a mixture of (R)-2-(3-((tert-butoxycarbonyl) (methyl) amino) pyrrolidin-1-yl)-4-ethoxypyrimidine-5-carboxylic acid (300 mg, 0.82 mmol) in DMF (10 mL) were added HATU (467 mg, 1.33 mmol) and DIEA (529 mg, 4.09 mmol) and the mixture was stirred at 40° C. for 0.5 h. Then 2-methylimidazo[1,2-a]pyridin-6-amine (130 mg, 0.82 mmol) was added and the mixture was stirred at 40° C. for 3 h. The mixture was diluted with H₂O (25 mL). The precipitate was filtered and dried in vacuo to give title product (340 mg, Y: 83.7%) as a yellow solid. ESI-MS (M+H)⁺: 496.1.

### Step 2: Preparation of (R)-4-ethoxy-2-(3-(methylamino) pyrrolidin-1-yl)-N-(2-methylimidazo[1,2-a]pyridin-6-yl)pyrimidine-5-carboxamide formate

A mixture of tert-butyl(R)-(1-(4-ethoxy-5-((2-methylimidazo[1,2-a]pyridin-6-yl) carbamoyl)pyrimidin-2-yl) pyrrolidin-3-yl) (methyl) carbamate (290 mg. 0.59 mmol) in 3M HCl/EA (10 mL) was stirred at rt for 1 h. The mixture was diluted with water (5 mL) and extracted with EA (10 mL×3). The aqueous phase was concentrated in vacuo. The crude was purified by prep-HPLC (0.05% FA in water/CH₃CN) to give title product (177.11 mg, Y: 76.6%) as a yellow solid. ESI-MS (M+H⁺): 396.0. ¹H NMR (400 MHZ, MeOD-d₄) δ 9.30 (s, 1H), 8.79 (s, 1H), 8.25 (s, 1H), 7.74 (s, 1H), 7.56 (d, J=9.6 Hz, 1H), 7.43 (d, J=9.5 Hz, 1H), 4.67 (q, J=6.9 Hz, 2H), 4.04-3.94 (m, 2H), 3.90-3.81 (m, 2H), 3.75-3.74 (m, 1H), 2.80 (s, 3H), 2.57-2.48 (m, 1H), 2.45 (s, 3H), 2.28-2.27 (m, 1H), 1.56 (t, J=7.0 Hz, 3H).

### Example 70. (R)-2-(3-(dimethylamino) pyrrolidin-1-yl)-4-ethoxy-N-(8-fluoro-2-methylimidazo[1,2-a]pyridin-6-yl)pyrimidine-5-carboxamide 4-methylbenzenesulfonate (Compound 290)

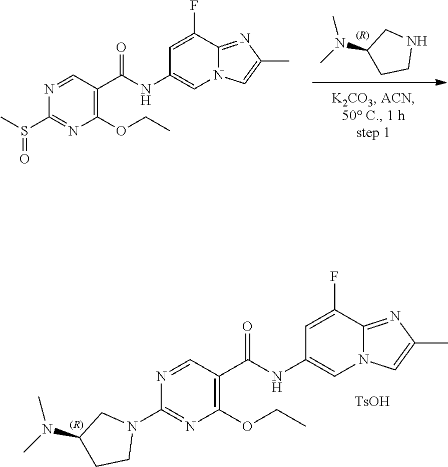

### Step 1: Preparation of (R)-2-(3-(dimethylamino) pyrrolidin-1-yl)-4-ethoxy-N-(8-fluoro-2-methylimidazo[1,2-a]pyridin-6-yl)pyrimidine-5-carboxamide 4-methylbenzenesulfonate

To a solution of 4-ethoxy-N-(8-fluoro-2-methylimidazo[1,2-a]pyridin-6-yl)-2-(methylsulfinyl)pyrimidine-5-carboxamide (303 mg, 0.80 mmol) in CH₃CN (6 mL) were added (R)—N,N-dimethylpyrrolidin-3-amine (110 mg, 0.96 mmol) and K₂CO₃ (266 mg, 1.93 mmol). The mixture was stirred at 50° C. for 1 h. The mixture was diluted with water (20 mL) and extracted with EA (20 mL). The organic layer was washed with brine, dried with Na₂SO₄ and evaporated. The crude was purified by pre-HPLC (0.05% NH₃.H₂O in water/ACN). The product was obtained by lyophiliazation and further diluted in leq 4-methylbenzenesulfonic acid and lyophilized to give title product (135 mg, 23.4%) as a yellow solid. ESI-MS (M+H)⁺: 428.2. ¹H NMR (400 MHZ, MeOD-d₄)δ 8.98 (s, 1H), 8.77 (s. 1H), 7.70-7.77 (m, 3H), 7.21 (d, J=8.0 Hz, 2H), 7.13 (d, J=11.9 Hz, 1H), 4.66 (q, J=7.0 Hz, 2H), 4.13-4.10 (m, 1H), 3.88-3.82 (m, 2H), 3.71-3.60 (m, 2H), 2.87 (s, 6H), 2.51-2.48 (m, 1H), 2.42 (s, 3H), 2.35 (s, 3H), 2.22-2.20 (m, 1H), 1.54 (t, J=7.1 Hz, 3H).

### Example 71. (S)-2-(3-(dimethylamino) pyrrolidin-1-yl)-4-ethoxy-N-(7-fluoro-2-methyl-2H-indazol-5-yl)pyrimidine-5-carboxamide formate (Compound 291)

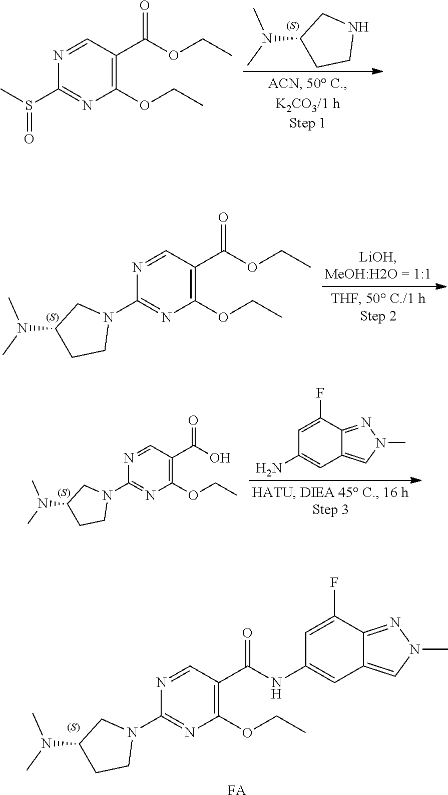

### Step 1: Preparation of ethyl(S)-2-(3-(dimethylamino) pyrrolidin-1-yl)-4-ethoxypyrimidine-5-carboxylate

To a mixture of ethyl 4-ethoxy-2-(methylsulfinyl)pyrimidine-5-carboxylate (3.0 g, 12.4 mmol) and(S)—N,N-dimethylpyrrolidin-3-amine (2 g, 18.60 mmol) in ACN (30 mL) was added K₂CO₃ (3.2 g, 37.20 mmol). The mixture was stirred at 50° C. for 1 h. The reaction mixture was diluted with water (50 mL), extracted with EtOAc (100 mL×3). The organic layer was washed with brine, dried over Na₂SO₄ and concentrated to give crude product (2.0 g, yield 52.6%) as a white solid. ESI-MS (M+H)⁺: 309.2.

### Step 2: Preparation of(S)-2-(3-(dimethylamino) pyrrolidin-1-yl)-4-ethoxypyrimidine-5-carboxylic acid

To a mixture of thyl(S)-2-(3-(dimethylamino) pyrrolidin-1-yl)-4-ethoxypyrimidine-5-carboxylate (2.0 g, 6.47 mmol) in MeOH (15 mL) and H₂O (15 mL) was added LiOH (1.33 g, 32.5 mmol). The reaction solution was stirred at 50° C. for 1 h. LCMS showed the reaction was completed. The reaction mixture was concentrated in vacuo to remove most THF. The mixture was adjusted to pH=5 with 1M HCl. The precipitate was filtered and dried in vacuo to give title product (1.2 g, Y: 66.6%) as a white solid. ESI-MS (M+H)⁺: 281.1.

### Step 3: Preparation of(S)-2-(3-(dimethylamino) pyrrolidin-1-yl)-4-ethoxy-N-(7-fluoro-2-methyl-2H-indazol-5-yl)pyrimidine-5-carboxamide formate

To a mixture of(S)-2-(3-(dimethylamino) pyrrolidin-1-yl)-4-ethoxypyrimidine-5-carboxylic acid (300 mg, 1.04 mmol) in DMF (5 mL) were added HATU (475 mg, 1.25 mmol), DIEA (537 mg, 4.16 mmol) and 2-methyl-2H-pyrazolo[3,4-b]pyridin-S-amine (226 mg, 1.25 mmol). The mixture was stirred at 45° C. for 16 h. The mixture was diluted with water (10 mL), extracted with EA (20 mL×3). The organic layer was washed with brine, dried over Na₂SO₄ and concentrated in vacuo to give the crude. The crude was purified by prep-HPLC (0.05% FA in water/CH₃CN) to give title product (248 mg, Y: 54.27%) as a yellow solid. ESI-MS (M+H⁺): 428.3. ¹H NMR (400 MHZ, MeOD-d₄)δ 8.76 (s, 1H), 8.34 (s, 1H), 8.23 (d, J=2.7 Hz, 1H), 7.89-7.83 (m, 1H), 7.23-7.16 (m, 1H), 4.64 (q, J=6.9 Hz, 2H), 4.21 (s, 3H), 4.07-4.01 (m, 1H), 3.90-3.89 (m, 1H), 3.62-3.53 (m, 2H), 3.48-3.47 (m, 1H), 2.67 (s, 6H), 2.47-2.38 (m, 1H), 2.15-2.03 (m, 1H), 1.55 (t, J=7.1 Hz, 3H).

### Example 72. (R)-2-(3-(dimethylamino) pyrrolidin-1-yl)-4-ethoxy-N-(7-fluoro-2-methyl-2H-indazol-5-yl)pyrimidine-5-carboxamide formate (Compound 292)

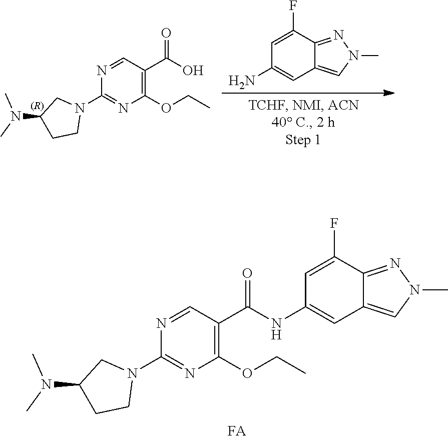

### Step 1: Preparation of (R)-2-(3-(dimethylamino) pyrrolidin-1-yl)-4-ethoxy-N-(7-fluoro-2-methyl-2H-indazol-5-yl)pyrimidine-5-car boxamide formate

To a mixture of (R)-2-(3-(dimethylamino) pyrrolidin-1-yl)-4-ethoxypyrimidine-5-carboxylic acid (250 mg, 0.893 mmol) in ACN (40 mL) were added TCFH (375 mg, 1.339 mmol), NMI (220 mg, 2.68 mmol) and 7-fluoro-2-methyl-2H-indazol-5-amine (147 mg,0.893 mmol) and the mixture was stirred at 40° C. for 2 h. The precipitate was filtered and purified by prep-HPLC (0.05% FA in water/CH&CN) to give title product (256 mg, Y: 67.2%) as a yellow solid. ESI-MS (M+H⁺): 428.1. ¹H NMR (400 MHZ, MeOD-d₄)δ 8.77 (s, 1H), 8.30 (s, 1H), 8.24 (d, J=2.6 Hz, 1H), 7.87 (d, J=1.2 Hz, 1H), 7.21 (d, J=12.7 Hz, 1H), 4.65 (q, J=7.0 Hz, 2H), 4.22 (s, 3H), 4.10-3.86 (m, 2H), 3.63-3.48 (m, 3H), 2.70 (s, 6H), 2.45-2.44 (m, 1H), 2.13-2.12 (m, 1H), 1.56 (t, J=7.1 Hz, 3H).

### Example 73. (S)-4-ethoxy-2-(3-(methylamino) pyrrolidin-1-yl)-N-(2-methylimidazo[1,2-a]pyridin-6-yl)pyrimidine-5-carboxamide formate (Compound 293)

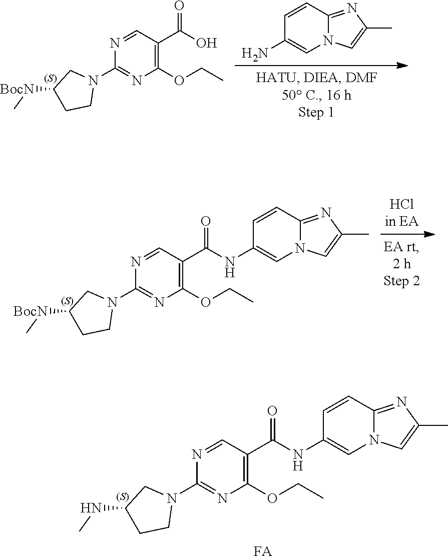

### Step 1: Preparation of tert-butyl(S)-(1-(4-ethoxy-5-((2-methylimidazo[1,2-a]pyridin-6-yl) carbamoyl)pyrimidin-2-yl) pyrrolidin-3-yl) (methyl) carbamate

To a mixture of 2-methylimidazo[1,2-a]pyridin-6-amine (200 mg, 1.23 mmol) in DMF (5 mL) were added DIEA (422 mg, 3.28 mmol), HATU (373 mg, 0.98 mmol), (S)-2-(3-((tert-butoxycarbonyl) (methyl) amino) pyrrolidin-1-yl)-4-ethoxypyrimidine-5-carboxylic acid (300 mg, 0.82 mmol). The mixture was stirred at 50° C. for 16 h. The reaction solution was diluted with water (10 mL) and extracted with EA (30 mL). The organic phase was washed with brine, dried and concentrated to obtain the title product (350 mg, Y: 86%) as a yellow solid. ESI-MS (M+H⁺): 496.1.

### Step 2: Preparation of(S)-4-ethoxy-2-(3-(methylamino) pyrrolidin-1-yl)-N-(2-methylimidazo[1,2-a]pyridin-6-yl)pyrimidine-5-carboxamide formate

To a mixture of tert-butyl(S)-(1-(4-ethoxy-5-((2-methylimidazo[1,2-a]pyridin-6-yl) carbamoyl)pyrimidin-2-yl) pyrrolidin-3-yl) (methyl) carbamate (350 mg, 0.71 mmol) in EA (5 mL) were added 3M HCl/EA (5 mL). The mixture was stirred at 25° C. for 2 h. The mixture was concentrated in vacuo, the residue was purified by prep-HPLC (0.05% FA in water/ACN) to give title product (95.55 mg, Y: 34%) as a yellow solid. ESI-MS (M+H⁺): 396.1. ¹H NMR (400 MHZ, MeOD-d₄)δ 9.26 (s, 1H), 8.79 (s, 1H), 8.29 (s, 1H), 7.70 (s, 1H), 7.52 (d, J=9.5 Hz, 1H), 7.36 (d, J=9.2 Hz, 1H), 4.67-4.65 (m, 2H), 4.03-3.85 (m, 4H), 3.75-3.74 (m, 1H), 2.80 (s, 3H), 2.53-2.48 (m, 1H), 2.44 (s, 3H), 2.28-2.27 (m, 1H), 1.56 (t, J=7.1 Hz, 3H).

### Example 74. (S)-2-(3-(dimethylamino) pyrrolidin-1-yl)-4-ethoxy-N-(8-fluoro-2-methylimidazo[1,2-a]pyridin-6-yl)pyrimidine-5-carboxamide formate (Compound 294)

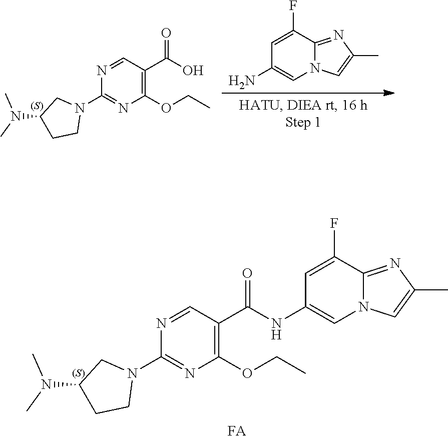

### Step 1: Preparation of(S)-2-(3-(dimethylamino) pyrrolidin-1-yl)-4-ethoxy-N-(8-fluoro-2-methylimidazo[ 1,2-a]pyridin-6-yl)pyrimidine-5-carboxamide formate

To a mixture of(S)-2-(3-(dimethylamino) pyrrolidin-1-yl)-4-ethoxypyrimidine-5-carboxylic acid (300 mg, 1.06 mmol) in DMF (5 mL) were added 8-fluoro-2-methylimidazo[1,2-a]pyridin-6-amine (211 mg, 1.28 mmol), HATU (608 mg, 1.6 mmol) and DIEA (691 mg, 5.36 mmol). The mixture was stirred at rt for 16 h. The mixture was diluted with water (5 mL) and the precipitate was filtered. The crude product was further purified by prep-HPLC (0.05% FA in water/CH₃CN) to give title product (168 mg, Y: 37.2%) as a white solid. ESI-MS (M+H)⁺: 428.6. ¹H NMR (400 MHZ, MeOD-d₄) δ 8.95 (d, J=1.5 Hz, 1H), 8.74 (s, 1H), 8.38 (s, 1H), 7.68 (d, J=2.3 Hz, 1H), 7.07 (dd, J=11.8, 1.6 Hz, 1H), 4.64 (q, J=6.8 Hz, 2H), 4.03-3.84 (m, 2H), 3.58-3.45 (m, 2H), 3.30-3.24 (m, 1H), 2.58 (s, 6H), 2.41 (s, 3H), 2.40-2.33 (m, 1H), 2.10-1.99 (m, 1H), 1.54 (t, J=7.1 Hz, 3H).

### Example 75. (R)-4-ethoxy-N-(7-methoxy-2-methyl-2H-indazol-5-yl)-2-(3-(methylamino) pyrrolidin-1-yl)pyrimidine-5-carboxamide 4-methylbenzenesulfonate (Compound 295)

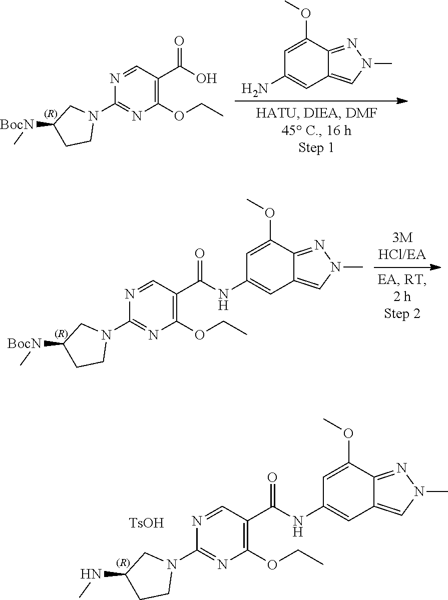

### Step 1: Preparation of tert-butyl(R)-(1-(4-ethoxy-5-((7-methoxy-2-methyl-2H-indazol-5-yl) carbamoyl)pyrimidin-2-yl) pyrrolidin-3-yl) (methyl) carbamate

To a mixture of (R)-2-(3-((tert-butoxycarbonyl) (methyl) amino) pyrrolidin-1-yl)-4-ethoxypyrimidine-5-carboxylic acid (400 mg, 1.09 mmol) in DMF (10 mL) were added HATU (497.04 mg, 1.31 mmol), DIEA (562.44 mg, 4.36 mmol) and 7-methoxy-2-methyl-2H-indazol-5-amino (231.87 mg, 1.31 mmol). The mixture was stirred at 45° C. for 16 h. The mixture was diluted with water (10 mL), extracted with EA (20 mL×3) The organic layer was washed with brine, dried over Na₂SO₄ and concentrated in vacuo to give title product (350 mg, 61.08%) as a yellow solid. ESI-MS (M+H⁺): 526.6.

### Step 2: Preparation of (R)-4-ethoxy-N-(7-methoxy-2-methyl-2H-indazol-5-yl)-2-(3-(methylamino) pyrrolidin-1-yl)pyrimidine-5-carboxamide 4-methylbenzenesulfonate

To a mixture of tert-butyl(R)-(1-(4-ethoxy-5-((7-methoxy-2-methyl-2H-indazol-5-yl) carbamoyl)pyrimidin-2-yl) pyrrolidin-3-yl) (methyl) carbamate (350 mg, 0.67 mmol) in EA (5 mL) was added 3 M HCLEA (8 mL). The mixture was stirred at rt for 2 h. The mixture was concentrated in vacuo. The crude was purified by prep-HPLC (0.05% 0.05% NH: H₂O in water/CH₃CN) to give title product as free base. The solid was dissolved in the solution of TsOH (1 eq) in water (3 mL) and lyophilized to give title product (215.57 mg, Y: 54.16%) as a yellow solid. ESI-MS (M+H⁺): 426.3. ¹H NMR (400 MHZ, DMSO-d₆) δ 9.53 (s, 1H), 8.75-8.63 (m, 3H), 8.21 (s, 1H), 7.71 (s, 1H), 7.47 (d, J=8.0 Hz, 2H), 7.11 (d, J=7.9 Hz, 2H), 6.79 (s, 1H), 4.57 (q, J=6.7 Hz, 2H), 4.11 (s, 3H), 3.91 (s, 3H), 3.88-3.86 (m, 2H), 3.78-3.71 (m, 2H), 3.67-3.61 (m, 1H), 2.68-2.65 (m, 3H), 2,33-2.30 (m, 1H), 2.29 (s, 3H), 2.22-2.12 (m, 1H), 1.48 (t, J=7.0 Hz, 3H).

### Example 76. 4-ethoxy-N-(8-fluoro-2-methylimidazo[1,2-a]pyridin-6-yl)-2-(4,7-diazaspiro[2.5]octan-7-yl)pyrimidine-5-carboxamide 2,2,2-trifluoroacetate (Compound 296)

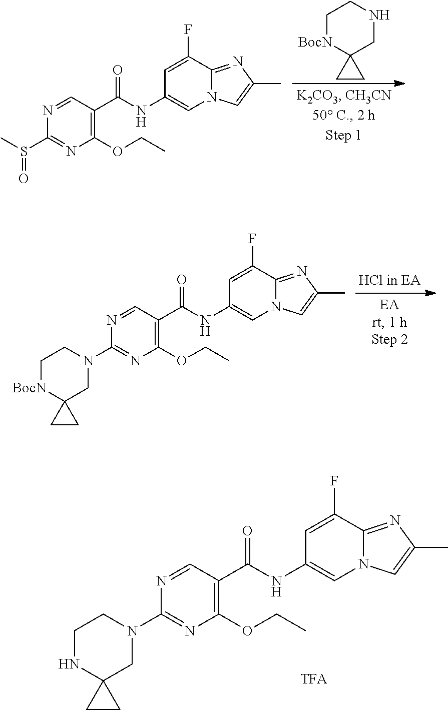

### Step 1: Preparation of tert-butyl 7-(4-ethoxy-5-((8-fluoro-2-methylimidazo[1,2-a]pyridin-6-yl) carbamoyl)pyrimidin-2-yl)-4,7-diazaspiro[2.5]octane-4-carboxylate

To a solution of tert-butyl 4,7-diazaspiro[2.5]octane-4-carboxylate (120 mg, 0.57 mmol) and 4-ethoxy-N-(8-fluoro-2-methylimidazo[1,2-a]pyridin-6-yl)-2-(methylsulfinyl)pyrimidine-5-carboxamide (277.4 mg, 0.74 mmol) in CH₃CN (10 mL) was added K₂CO₃ (156.2 mg, 1.13 mmol). The mixture was stirred at 50° cfor 2 h. The reaction mixture was diluted with H₂O (20 mL), extracted with EA (50 mL×3). The organic layer was washed with brine, dried over Na₂SO₄ and evaporated to give crude product (120 mg, yield 40%) as a brown solid, which was used to next step without further purification. ESI-MS (M+H)⁺: 526.3.

### Step 2: Preparation of 4-ethoxy-N-(8-fluoro-2-methylimidazo[1,2-a]pyridin-6-yl)-2-(4,7-diazaspiro[2.5]octan-7-yl)pyrimidine-5-carboxamide 2,2,2-trifluoroacetate

To a mixture of tert-butyl 7-(4-ethoxy-5-((8-fluoro-2-methylimidazo[1,2-a]pyridin-6-yl) carbamoyl)pyrimidin-2-yl)-4,7-diazaspiro[2.5]octane-4-carboxylate (120 mg, 0.23 mmol) in EA (5 mL) was added EA/HCl (5 mL). The mixture was stirred at rt for 1 h. The reaction mixture was concentrated in vacuo. The residue was purified by pre-HPLC (0.1% TFA in water/CH₃CN) to give title product (43 mg, 34.8%) as a white solid. ESI-MS (M+H)⁺: 426.2. ¹H NMR (400 MHZ, MeOD-d₄)δ 9.40 (d, J=1.2 Hz, 1H), 8.81 (s, 1H), 8.12-8.06 (m, 1H), 7.93-7.85 (m, 1H), 4.70-4.57 (m, 2H), 4.31-4.23 (m, 2H), 4.11 (s, 2H), 3.47-3.40 (m, 2H), 2.60-2.53 (m, 3H), 1.53 (t, J=7.1 Hz, 3H), 1.16-1. 11 (m, 2H), 1.10-1.03 (m, 2H).

### Example 77. (R)-4-ethoxy-N-(8-fluoro-2-methylimidazo[1,2-a]pyridin-6-yl)-2-(3-((methylamino) methyl) pyrrolidin-1-yl)pyrimidine-5-carboxamide 4-methylbenzenesulfonate (Compound 297)

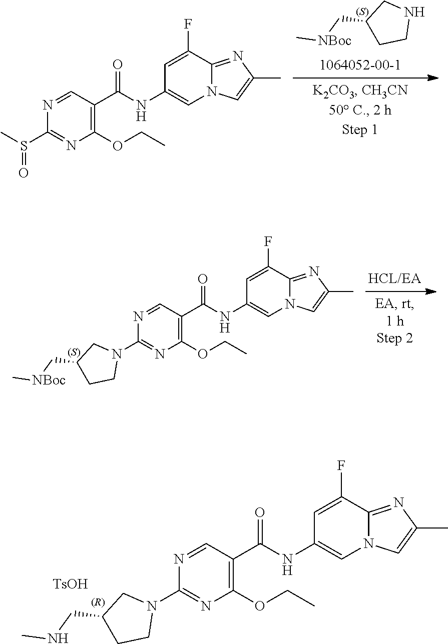

### Step 1: Preparation of tert-butyl(S)-((1-(4-ethoxy-5-(8-fluoro-2-methylimidazo[1,2-a]pyridin-6-yl) carbamoyl)pyrimidin-2-yl) pyrrolidin-3-yl)methyl) (methyl) carbamate

To a solution of tert-butyl(S)-methyl(pyrrolidin-3-ylmethyl) carbamate (400 mg, 1.87 mmol) and 4-ethoxy-N-(8-fluoro-2-methylimidazo[1,2-a]pyridin-6-yl)-2-(methylsulfinyl)pyrimidine-5-carboxamide (1.06 g, 2.80 mmol) in CH₃CN (15 mL) was added K₂CO₃ (142 mg, 1.03 mmol). The mixture was stirred at 50° C. for 2 h. The reaction mixture was diluted with H₂O (25 mL), extracted with DCM (60 mL×3). The organic layer was washed with brine, dried over Na₂SO₄ and evaporated to give crude title product (400 mg, yield 40.6%) as a brown solid, which was used to next step without further purification. ESI-MS (M+H)⁺: 528.2

### Step 2: Preparation of (R)-4-ethoxy-N-(8-fluoro-2-methylimidazo[1,2-a]pyridin-6-yl)-2-(3-((methylamino) methyl) pyrrolidin-1-yl)pyrimidine-5-carboxamide 4-methylbenzenesulfonate

To a mixture of tert-butyl(S)-((1-(4-ethoxy-5-((8-fluoro-2-methylimidazo[1,2-a]pyridin-6-yl) carbamoyl)pyrimidin-2-yl) pyrrolidin-3-yl)methyl) (methyl) carbamate (400 mg, 0.76 mmol) in EA (10 mL) was added EA/HCl (10 mL). The mixture was stirred at rt for 1 h. The reaction mixture was concentrated in vacuo. The residue was purified by pre-HPLC (0.1% NH₃H₂O in water/CH₃CN) to give title product (115 mg, 0.27 mmoL) as a free base. This product was dissolved with aqueous TsOH (1.0 eq) and lyophilized to give title product (143 mg, 31.5%). ESI-MS (M+H)⁺: 428.3. ¹H NMR (400 MHZ, MeOD-d₄)δ 8.97 (s, 1H), 8.77 (s, 1H), 7.72-7.68 (m, 3H), 7.25-7.21 (m, 2H), 7.16-7.11 (m, 1H), 4.67 (q, J=7.0 Hz, 2H), 3.98-3.82 (m, 2H), 3.65-3.59 (m, 1H), 3.49-3.46 (m, 1H), 3.16-3.13 (m, 2H), 2.75 (s, 3H), 2.71-2.63 (m, 1H), 2.42 (s, 3H), 2.36 (s, 3H), 2.32-2.25 (m, 1H), 1.90-1.80 (m, 1H), 1.54 (t, J=7.1 Hz, 3H).

### Example 78. (S)-4-ethoxy-N-(7-methoxy-2-methyl-2H-indazol-5-yl)-2-(3-(methylamino) pyrrolidin-1-yl)pyrimidine-5-carboxamide 4-methylbenzenesulfonate (Compound 298)

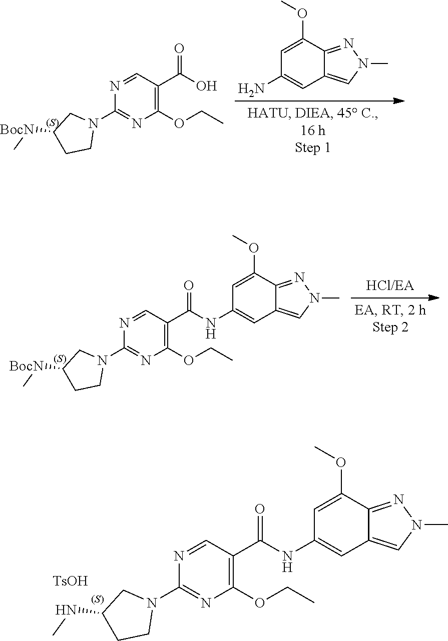

### Step 1: Preparation of tert-butyl(S)-(1-(4-ethoxy-5-((7-methoxy-2-methyl-2H-indazol-5-yl) carbamoyl)pyrimidin-2-yl) pyrrolidin-3-yl) (methyl) carbamate

To a mixture of(S)-2-(3-((tert-butoxycarbonyl) (methyl) amino) pyrrolidin-1-yl)-4-ethoxypyrimidine-5-carboxylic acid (400 mg, 1.09 mmol) in DMF (10 mL) were added HATU (497.04 mg, 1.31 mmol), DIEA (562.44 mg. 4.36 mmol) and 7-methoxy-2-methyl-2H-indazol-5-amine (231.87 mg, 1.31 mmol). The mixture was stirred at 45° C. for 16 h. The mixture was diluted with water (10 mL), extracted with EA (20 mL×3). The organic layer was washed with brine, dried over Na₂SO₄ and concentrated in vacuo to give title product (400 mg, 69.69%) as a yellow solid. ESI-MS (M+H⁺): 526.3.

### Step 2: Preparation of(S)-4-ethoxy-N-(7-methoxy-2-methyl-2H-indazol-5-yl)-2-(3-(methylamino) pyrrolidin-1-yl)pyrimidine-5-carboxamide 4-methylbenzene sulfonate

To a mixture of tert-butyl(S)-(1-(4-ethoxy-5-((7-methoxy-2-methyl-2H-indazol-5-yl) carbamoyl)pyrimidin-2-yl) pyrrolidin-3-yl) (methyl) carbamate (400 mg, 0.76 mmol) in EA (4 mL) was added 3 M HCVEA (10 mL). The mixture was stirred at rt for 2 h. The mixture was concentrated in vacuo. The crude was purified by prep-HPLC (0.05% NH₃·H₂O in water/CH₃CN) to give title product as a free base. The solid was dissolved in the solution of TsOH (1 eq) in water (3 mL) and lyophilized to give title product (399.62 mg, Y: 87.83%) as a yellow solid. ESI-MS (M+H⁺): 426.4. ¹H NMR (400 MHZ, DMSO-d₆) δ 9.52 (s, 1H), 8.68 (s, 1H), 8.21 (s, 1H), 7.71 (s, 1H), 7.47 (d, J=8.0 Hz, 2H), 7.11 (d, J=8.0 Hz, 2H), 6.79 (s, 1H), 4.57 (q, J=6.7 Hz, 2H), 4.11 (s, 3H), 3.91 (s, 3H), 3.87-3.82 (m, 2H), 3.76-3.70 (m, 2H), 3.67-3.62 (m, 1H), 2.63 (s, 3H), 2.36-2.32 (m, 1H), 2.29 (s, 3H), 2.17-2.10 (m, 1H), 1.48 (t, J=7.0 Hz, 3H).

### Example 79. 4-ethoxy-N-(7-fluoro-2-methyl-2H-indazol-5-yl)-2-(4,7-diazaspiro[2.5]octan-7-yl)pyrimidine-5-carboxamide formate (Compound 299)

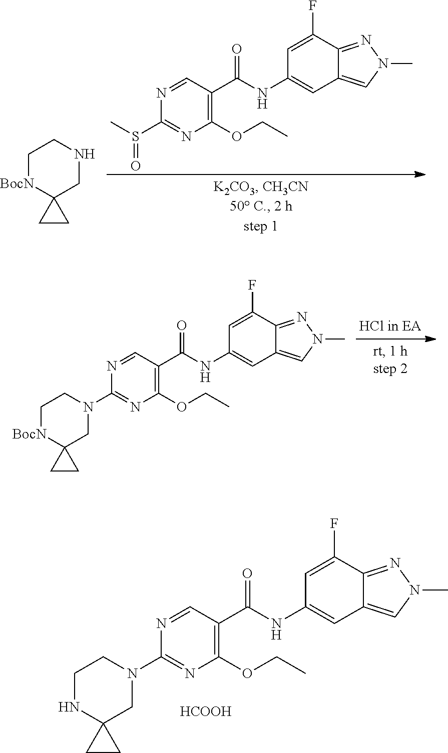

### Step 1: Preparation of tert-butyl 7-(4-ethoxy-5-(7-fluoro-2-methyl-2H-indazol-5-yl) carbamoyl)pyrimidin-2-yl)-4,7-diazaspiro[2.5]octane-4-carboxylate

To a mixture of tert-butyl 4,7-diazaspiro[2.5]octane-4-carboxylate (100 mg, 0.47 mmol), 4-ethoxy-N-(7-fluoro-2-methyl-2H-indazol-5-yl)-2-(methylsulfinyl)pyrimidine-5-carboxamide (711.32 mg, 1.89 mmol) in ACN (10 mL) was added K₂CO₃ (129.72 mg, 0.94 mmol). The mixture was stirred at 50° C. for 2 h. The precipitate was filtered, the filtrate was concentrated in vacuo to give title product (120 mg, 49%) as a white solid. ESI-MS (M+H⁺): 526.2.

### Step 2: Preparation of 4-ethoxy-N-(7-fluoro-2-methyl-2H-indazol-5-yl)-2-(4,7-diazaspiro[2.5]octan-7-yl)pyrimidine-5-carboxamide formate

A mixture of tert-butyl 7-(4-ethoxy-5-((7-fluoro-2-methyl-2H-indazol-5-yl) carbamoyl)pyrimidin-2-yl)-4,7-diazaspiro[2.5]octane-4-carboxylate (120 mg, 0.23 mmol) in 3 M EA/HCl (5 mL) was stirred at rt for 1 h. The mixture was concentrated in vacuo, the residue was purified by prep-HPLC (0.05% FA in water/CH₃CN) to give title product (23.45 mg, 24%) as a yellow solid. ESI-MS (M+H⁺): 426.1. ¹H NMR (400 MHZ, MeOD-d₄) δ 8.78 (s, 1H), 8.41-8.18 (m, 2H), 7.87 (s, 1H), 7.22 (d, J=12.8 Hz, 1H), 4.64-4.56 (m, 2H), 4.22 (s, 3H), 4.09-4.04 (m, 2H), 3.91 (s, 2H), 3.18-3.12 (m, 2H), 1.54 (t, J=7.0 Hz, 3H), 0.82 (br s, 4H).

### Example 80. 2-(4-((dimethylamino) methyl) piperidin-1-yl)-4-ethoxy-N-(8-fluoro-2-methylimidazo[1,2-a]pyridin-6-yl)pyrimidine-5-carboxamide formate (Compound 300)

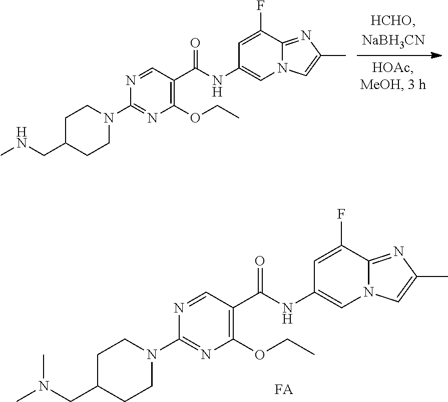

### Step 1: Preparation of 2-(4-((dimethylamino) methyl) piperidin-1-yl)-4-ethoxy-N-(8-fluoro-2-methylimidazo[1,2-a]pyridin-6-yl)pyrimidine-5-carboxamide formate

To a mixture of 4-ethoxy-N-(8-fluoro-2-methylimidazo[1,2-a]pyridin-6-yl)-2-(4-((methylamino) methyl) piperidin-1-yl)pyrimidine-5-carboxamide (70 mg, 0.114 mmol) in MeOH (4 mL) were added (HCHO) n (17.1 mg, 0.57 mmol) and HOAc (20.55 mg, 0.34 mmol). The mixture was stirred at rt for 1 h. Then NaBH₃CN (36 mg, 0.571 mmol) was added and the mixture was stirred at rt for 2 h. The mixture was concentrated. The crude was purified by prep-HPLC (0.05% FA in water/ACN) to give title product (18 mg, Y: 57.2%) as a yellow solid. ESI-MS (M+H)⁺: 456.2. ¹H NMR (400 MHZ, MeOD-d₄)δ 8.99 (s, 1H), 8.78 (s, 1H), 8.56 (s, 1H), 7.72 (s, 1H), 7.15 (d, J=11.8 Hz, 1H), 4.90-4.85 (m, 2H), 4.65 (q, J=7.0 Hz, 2H), 3.05 (t, J=12.9 Hz, 2H), 2.64-2.62 (m, 8H), 2.44 (s, 3H), 2.07 (br s, 1H), 1.89-1.85 (m, 2H), 1.56 (t, J=7.0 Hz, 3H), 1.25-1.22 (m, 2H).

### Example 81. N-(7-fluoro-2-methyl-2H-indazol-5-yl)-2-(piperazin-1-yl)-4-propoxypyrimidine-5-carboxamide (Compound 301)

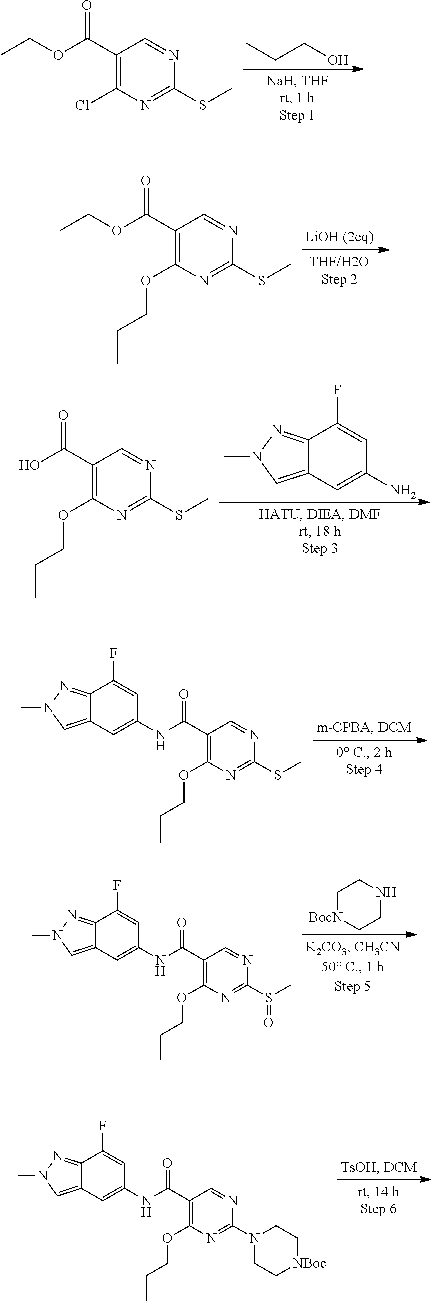

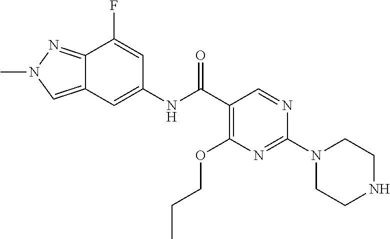 [SMILES: CCCOc1nc(N2CCNCC2)ncc1C(=O)Nc1cc(F)c2nn(C)cc2c1]

### Step 1: ethyl 2-(methylthio)-4-propoxypyrimidine-5-carboxylate

To a solution of propan-1-ol (0.54 g, 9.00 mmol) in THF (10 mL) was added 60% NaH (0.36 g, 9.00 mmol) and stirred at rt for 0.5 h. Then a solution of ethyl 4-chloro-2-(methylthio)pyrimidine-5-carboxylate (1.40 g, 6.00 mmol) in THF (5 ml . . . ) was added and stirred at rt under Ar atmosphere for 1 h. The mixture was quenched by NH₄Cl (aq) (20 mL) and extracted with EA (80 mL×2). The organics were washed with water (80 ml), brine (80 mL), dried over sodium sulfate, filtered, and concentrated to give title product (1.14 g, 74% yield) as a yellow solid. ESI-MS (M+H)⁺: 257.1.

### Step 2:2-(methylthio)-4-propoxypyrimidine-5-carboxylic acid

To a solution of ethyl 2-(methylthio)-4-propoxypyrimidine-5-carboxylate (0.7 g, 2.70 mmol) in THF (4 mL) and H₂O (4 mL) was added LiOH.H₂O (227 mg, 5.40 mmol). The mixture was stirred at rt under Ar atmosphere for 3 h. The mixture was acidified by 1N HCl to pH˜4, and extracted with EA (50 mL×2). The organics were washed with water (60 mL) and brine (50 mL), dried over sodium sulfate, filtered and concentrated to give title product (170 mg, crude) as a yellow solid. ESI-MS (M+H)⁺: 229.1.

### Step 3: N-(7-fluoro-2-methyl-2H-indazol-5-yl)-2-(methylthio)-4-propoxypyrimidine-5-carboxamide

To a mixture of 2-(methylthio)-4-propoxypyrimidine-5-carboxylic acid (170 mg 0.74 mmol), 7-fluoro-2-methyl-2H-indazol-5-amine (135 mg, 0.82 mmol) and HATU (420 mg, 1.10 mmol) in DMF (4 mL) was added DIEA (0.4 mL, 2.20 mmol) and the mixture was stirred at rt under Ar atmosphere for 16 h. The mixture was diluted with water (20 mL), extracted with EA (40 mL×2), washed with water (10 mL) and brine (10 mL), dried over sodium sulfate, filtered and concentrated. The crude was purified by silica gel column (PE/EA=5:1) to give title product (100 mg, 36% yield) as a yellow solid. ESI-MS (M+H)⁺: 376.2.

### Step 4: N-(7-fluoro-2-methyl-2H-indazol-5-yl)-2-(methylsulfinyl)-4-propoxypyrimidine-5-carboxamide

To a solution of N-(7-fluoro-2-methyl-2H-indazol-5-yl)-2-(methylthio)-4-propoxypyrimidine-5-carboxamide (100 mg, 0.27 mmol) in DCM (5 mL) was added m-CPBA (215 mg, 1.25 mmol) and the mixture was stirred under Ar atmosphere for 2 h. The mixture was diluted with DCM (20 mL), washed with H₂O (5 mL) and brine (5 mL), dried over sodium sulfate, filtered, and concentrated. The crude was purified by silica gel column (PE/EA=4:1) to give title product (80 mg. 74% yield) as a yellow solid. ESI-MS (M+H)⁺: 392.2.

### Step 5: tert-butyl 4-(5-((7-fluoro-2-methyl-2H-indazol-5-yl) carbamoyl)-4-propoxypyrimidin-2-yl) piperazine-1-carboxylate

To a mixture of N-(7-fluoro-2-methyl-2H-indazol-5-yl)-2-(methylsulfinyl)-4-propoxypyrimidine-5-carboxamide (80 mg, 0.20 mmol) and tert-butyl piperazine-1-carboxylate (75 mg, 0.40 mmol) in MeCN (5 mL) was added K₂CO₃ (56 mg, 0.41 mmol) and the mixture was stirred at 50° C. under Ar atmosphere for 1 h. The mixture was diluted with water (25 mL), extracted with EA (30 mL×2). The organics were washed with water (15 mL) and brine (15 mL), dried over sodium sulfate, filtered, concentrated. The crude was purified by silica gel column (PE/EA=3:1) to give title product (80 mg, 80% yield) as a yellow solid. ESI-MS (M+H)⁺: 514.4.

### Step 6: N-(7-fluoro-2-methyl-2H-indazol-5-yl)-2-(piperazin-1-yl)-4-propoxypyrimidine-5-carboxamide

To a solution of tert-butyl 4-(5-((7-fluoro-2-methyl-2H-indazol-5-yl) carbamoyl)-4-propoxypyrimidin-2-yl) piperazine-1-carboxylate (80 mg, 0.16 mmol) in DCM (4 mL) was added TsOH (149 mg, 0.78 mmol) and the mixture was stirred at rt for 14 h. The mixture was concentrated and purified by pre-HPLC (0.1% NH₄HCO3 in water/CH₃CN) to give title product (30 mg, 30% yield) as a yellow solid. ESI-MS (M+H)⁺: 414.3. ¹H NMR (400 MHZ, DMSO-d₆) δ 9.59 (s, 1H), 8.62 (s, 1H), 8.42 (d, J=2.8 Hz, 1H), 7.96 (d, J=1.2 Hz, 1H), 7.28 (dd, J=13.6, 1.2 Hz, 1H), 4.42 (t, J=6.4 Hz, 2H), 4.18 (s, 3H), 3.76 (t, J=4.8 Hz, 4H), 2.77 (t, J=4.8 Hz, 4H), 1.88-1.82 (m, 2H), 1.01 (t, J=7.6 Hz, 3H).

### Example 82. 4-(2,2-difluoroethoxy)-N-(7-fluoro-2-methyl-2H-indazol-5-yl)-2-(piperazin-1-yl)pyrimidine-5-carboxamide (Compound 302)

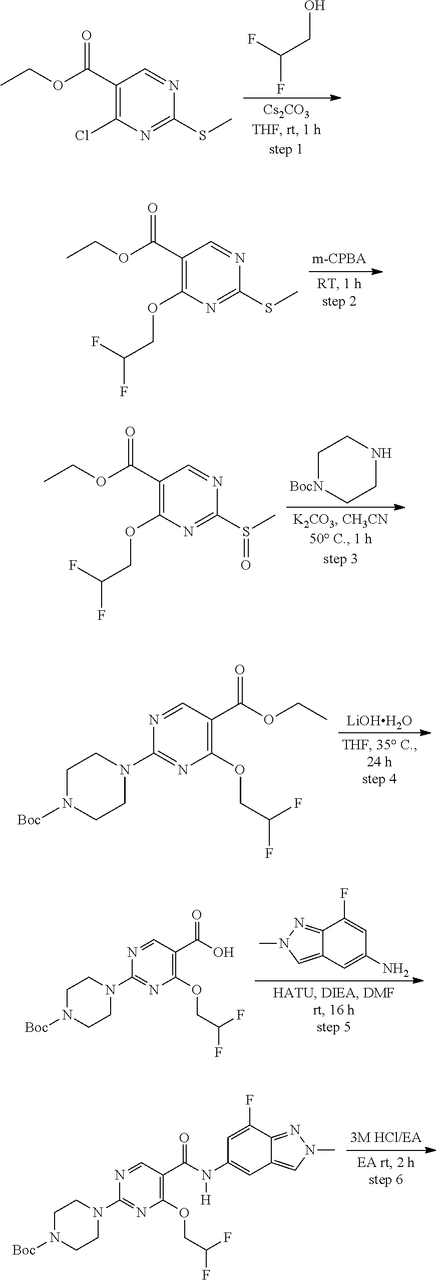

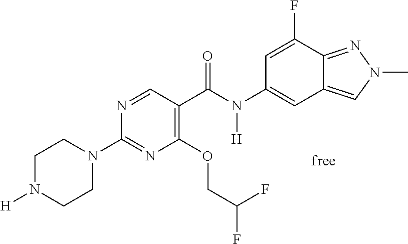 [SMILES: Cn1cc2cc(NC(=O)c3cnc(N4CCNCC4)nc3OCC(F)F)cc(F)c2n1]

### Step 1: Preparation of ethyl 4-(2,2-difluoroethoxy)-2-(methylthio)pyrimidine-5-carboxylate

To a solution of ethyl 4-chloro-2-(methylthio)pyrimidine-S-carboxylate (3 g, 0.0129 mol) in THF (30 mL) were added 2,2-difluoroethan-1-ol (2.1 g, 0.026 mol) and Cs₂CO₃ (4.2 g. 0.013 mol). The mixture was stirred at rt for 1 h. Filtrated and concentrated in vacuo to give crude title product (3.6 g, 97%) as a colorless oil, which was used to next step without further purification. ESI-MS (M+H)⁺: 279.0. ¹H NMR (400 MHZ, CDCl₃)δ 8.90 (s, 1H), 6.16 (t. J=55.1 Hz, 1H), 4.68-4.67 (m, 2H), 4.36 (q, J=7.1 Hz, 2H), 2.58 (s, 3H), 1.37 (t, J=7.1 Hz, 3H).

### Step 2: Preparation of ethyl 4-(2,2-difluoroethoxy)-2-(methylsulfinyl)pyrimidine-5-carboxylate

To a solution of ethyl 4-(2,2-difluoroethoxy)-2-(methylthio)pyrimidine-5-carboxylate (3.6 g, 1.29 mmol) in DCM (50 mL) was added m-CPBA (3.34 g, 1.94 mmol). The mixture was stirred at rt for 1 h. The mixture was washed with water and bine, dried over Na₂SO₄ and concentrated in vacuo to give crude title product as a white solid. ESI-MS (M+H)⁺: 295.1.

### Step 3: Preparation of ethyl 2-(4-(tert-butoxycarbonyl) piperazin-1-yl)-4-(2,2-difluoroethoxy) pyrimidine-5-carboxylate

To a solution of rude ethyl 4-(2,2-difluoroethoxy)-2-(methylsulfinyl)pyrimidine-5-carboxylate from above step in CH&CN (60 mL) were added K₂CO₃ (6.1 g, 0.044 mol) and tert-butyl piperazine-1-carboxylate (6.17 g, 0.033 mol), the mixture was stirred at 50° C. for 1 h. The reaction mixture was diluted with water (30 mL) and extracted with EtOAc (30 mL×3). The organic layer was washed with brine, dried over Na₂SO₄ and concentrated in vacuo to give title compound (3.4 g. 36.9% for two steps) as a colorless oil. ESI-MS (M+H)⁺: 417.2. JH NMR (400 MHZ, CDCl₃) δ 8.77 (s, 1H), 6.12 (t, J=54.7 Hz, 1H), 4.59-4,56 (m, 2H), 4.30 (q, J=7.1 Hz, 2H), 3.86 (br.s, 4H), 3.54-3.49 (m, 4H), 1.49 (s, 9H), 1.34 (t, J=7.1 Hz, 3H).

### Step 4: Preparation of 2-(4-(tert-butoxycarbonyl) piperazin-1-yl)-4-(2,2-difluoroethoxy) pyrimidine-5-carboxylic acid

To a mixture of ethyl 2-(4-(tert-butoxycarbonyl) piperazin-1-yl)-4-(2,2-difluoroethoxy) pyrimidine-5-carboxylate (3.4 g, 0.82 mmol) in THF: H₂O (10 mL: 10 mL) was added LiOH (0.69 g, 1.63 mmol), the mixture was stirred at 35° C. for 24 h. The reaction mixture was concentrated in vacuo to remove THF. The mixture was adjusted to pH=5 with 1M HCl and the precipitate was filtered and dried under vacuum to give product (2.2 g, 70.97%) as a white solid. ESI-MS (M+H)⁺: 389.2. ¹H NMR (400 MHZ, CDCl₃)δ 8.87 (s, 1H), 6.17 (t, J=55.1 Hz, 1H), 4.66-4.62 (m, 2H), 3.90 (br s, 4H), 3.53 (br s, 4H), 1.49 (s, 9H).

### Step 5: Preparation of tert-butyl 4-(4-(2,2-difluoroethoxy)-5-((7-fluoro-2-methyl-2H-indazol-5-yl) carbamoyl)pyrimidin-2-yl) piperazine-1-carboxylate

To a solution of 2-(4-(tert-butoxycarbonyl) piperazin-1-yl)-4-(2,2-difluoroethoxy) pyrimidine-5-carboxylic acid (120 mg, 0.31 mmol) in DMF (4 mL) were added 7-fluoro-2-methyl-2H-indazol-5-amine (76.5 mg, 0.464 mmol), HATU (176.3 mg, 0.464 mmol) and DIEA (159.59 mg, 1.24 mmol). The mixture was stirred at rt for 16 h. The mixture was diluted with water (20 mL), the precipitate was filtered and dried in vacuo to give crude title product (130 mg, Y: 78.79%) as a white solid. ESI-MS (M+H)⁺: 536.3. 1H NMR (400 MHZ, CDCl₃) δ 9.05 (s, 1H), 8.03 (s, 1H), 7.89 (s, 1H), 6.98 (d, J=12.2 Hz, 1H), 6.25 (t, J=55.1 Hz 1H), 4.75-4.73 (m, 2H), 4.22 (s, 3H), 3.89 (br s, 4H), 3.55-3.51 (m, 4H), 1.50 (s, 9H).

### Step 6: Preparation of 4-(2,2-difluoroethoxy)-N-(7-fluoro-2-methyl-2H-indazol-5-yl)-2-(piperazin-1-yl)pyrimidine-5-carboxamide

To a mixture of tert-butyl 4-(4-(2,2-difluoroethoxy)-5-((7-fluoro-2-methyl-2H-indazol-5-yl) carbamoyl)pyrimidin-2-yl) piperazine-1-carboxylate (130 mg, 0.243 mmol) in EA (4 mL) was added 3M HCl/EA (4 mL), the mixture was stirred at rt for 2 h. The mixture was concentrated in vacuo, the residue was purified by prep-HPLC (0.05% NH₃. H₂O in water/CH₃CN) to give title product (50 mg, Y: 47.6%) as a white solid. ESI-MS (M+H⁺): 436.2. ¹H NMR (400 MHZ, DMSO-d₆) δ 9.68 (s, 1H), 8.71 (s, 1H), 8.43 (d, J=2.8 Hz, 1H), 7.98 (d, J=1.2 Hz, 1H), 7.24 (d, J=13.2 Hz, 1H), 6.77-6.36 (m, 1H), 4.80-4.78 (m, 2H), 4.18 (s, 3H), 4.06 (br s, 4H), 3.20 (br s, 4H)

### Example 83. 4-ethoxy-N-(8-fluoro-2-methylimidazo[1,2-a]pyridin-6-yl)-2-(2-methyl-2,7-diazaspiro[3.5]nonan-7-yl)pyrimidine-5-carboxamide (Compound 303)

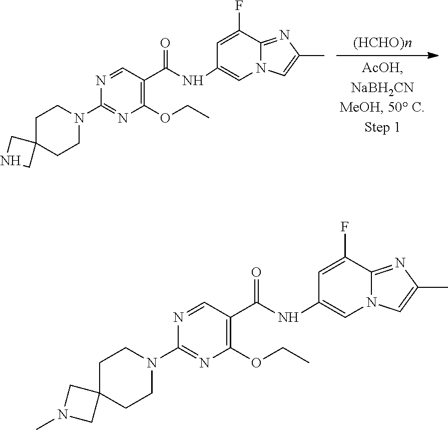

### Step 1: Preparation of 4-ethoxy-N-(8-fluoro-2-methylimidazo[1,2-a]pyridin-6-yl)-2-(2-methyl-2,7-diazaspiro/3.5}nonan-7-yl)pyrimidine-5-carboxamide

To a solution of 4-ethoxy-N-(8-fluoro-2-methylimidazo[1,2-a]pyridin-6-yl)-2-(2,7-diazaspiro[3.5]nonan-7-yl)pyrimidine-5-carboxamide (80 mg, 0.18 mmol) and AcOH (32 mg, 0.54 mmol) in MeOH (5 mL) were added NaBH₃CN (56 mg, 0.90 mmol) and (HCHO); (27 mg, 0.90 mmoL). The mixture was stirred at 50° C. for 5 h. The mixture was filtered and the filtrate was concentrated in vacuo. The residue was purified by prep-HPLC (0.1% NH₃. H₂O in water/ACN) to give title product (14.51 mg, Y: 15.95%) as a yellow solid. ESI-MS (M+H⁺): 454.3. ¹H NMR (400 MHZ, MeOD-d₄)δ 8.97 (s, 1H), 8.76 (s, 1H), 7.71 (s, 1H), 7.13 (d, J=11.7 Hz, 1H), 4.63 (q, J=7.2 Hz, 2H), 3.93-3.86 (m, 8H), 2.87 (s, 3H), 2.41 (s, 3H), 1.94-1.88 (m, 4H), 1.53 (t, J=7.0 Hz, 3H).

### Example 84. (S)-4-ethoxy-N-(8-fluoro-2-methylimidazo[1,2-a]pyridin-6-yl)-2-(3-((methylamino) methyl) pyrrolidin-1-yl)pyrimidine-5-carboxamide 4-methylbenzenesulfonate (Compound 304)

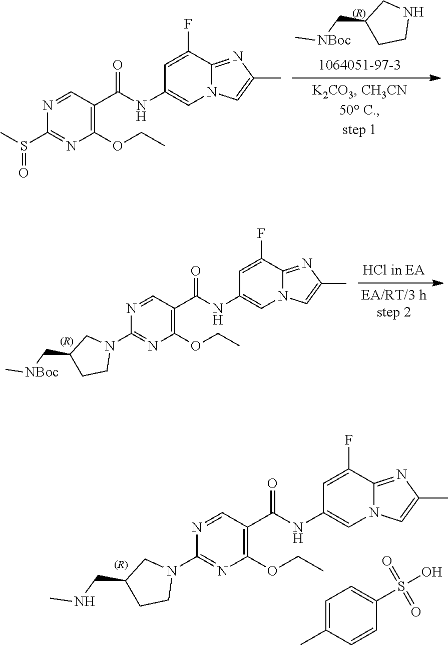

### Step 1: Preparation of tert-butyl(R)-((1-(4-ethoxy-5-((8-fluoro-2-methylimidazo[1,2-a]pyridin-6-yl) carbamoyl)pyrimidin-2-yl) pyrrolidin-3-yl)methyl) (methyl) carbamate

To a mixture of 4-ethoxy-N-(8-fluoro-2-methylimidazo[1,2-a]pyridin-6-yl)-2-(methylsulfinyl)pyrimidine-5-carboxamide (200 mg, 0.53 mmol) and (R)-tert-butyl methyl(pyrrolidin-3-ylmethyl) carbamate (114 mg, 0.53 mmol) in ACN (10 mL) was added K₂CO₃ (220 mg, 1.59 mmol). The mixture was stirred at 50° C. for 1 h. LCMS showed the reaction was completed. The reaction mixture was diluted with H₂O (10 mL) and extracted with EA (15 mL×3). The organic layer was washed with brine, dried over Na₂SO₄ and evaporated. The crude was purified by silica gel column chromatography (PE:EA=1:4) to give title product (100 mg, 35.8%) as a white solid. ESI-MS (M+H)⁺: 528.5. ¹H NMR (400 MHz, CDCl₃) δ 9.24 (s, 1H), 9.11 (s, 1H), 8.99 (d, J=3.5 Hz, 1H), 7.39 (d, J=2.6 Hz, 1H), 6.57 (d, J=10.7 Hz, 1H), 4.66-4.58 (m, 2H), 3.85-3.72 (m, 2H), 3.70-3.55 (m, 1H), 3.43-3.31 (m, 2H), 3.32-3.20 (m, 1H), 2.92 (s, 3H), 2.62-2.60 (m, 1H), 2.46 (s, 3H), 2.18-2.07 (m, 1H), 1.59-1.55 (m, 3H), 1.45 (s, 9H).

### Step 2: Preparation of(S)-4-ethoxy-N-(8-fluoro-2-methylimidazo[1,2-a]pyridin-6-yl)-2-(3-((methylamino) methyl) pyrrolidin-1-yl)pyrimidine-5-carboxamide 4-methylbenzenesulfonate

A mixture of tert-butyl(R)-((1-(4-ethoxy-5-((8-fluoro-2-methylimidazo[1,2-a]pyridin-6-yl) carbamoyl)pyrimidin-2-yl) pyrrolidin-3-yl)methyl) (methyl) carbamate (100 mg, 0.19 mmol) in 3M HCl/EA (2 mL) was stirred at rt for 3 h. LCMS showed the reaction was completed. The mixture was diluted with H₂O (5 mL) and extracted with EA (10 mL). The aqueous layer was concentrated in vacuo and purified by reversed phase column (0.05% NH₃.H₂O in water/ACN) to give the product as free base. The solid was dissolved in aqueous p-Toluenesulfonic acid solution (1 eq) and lyophilized to give title product (100 mg, 88%) as a yellow solid. ESI-MS (M+H)⁺: 428.2. 1 H NMR (400 MHZ, DMSO-d₆)δ 9.57 (s, 1H), 9.10-9.04 (m, 1H), 8.65 (s, 1H), 7.89 (d, J=2.7 Hz, 1H), 7.48 (d, J=8.1 Hz, 2H), 7.24 (d, J=12.2 Hz, 1H), 7.12 (d, J=7.8 Hz, 2H), 4.56 (q, J=7.0 Hz, 2H), 3.86-3.79 (m, 1H), 3.76-3.68 (m, 1H), 3.58-3.47 (m, 2H), 2.98 (d, J=6.8 Hz, 2H), 2.57 (s, 3H), 2.53-2.50 (m, 1H), 2.34 (s, 3H), 2.29 (s, 3H), 2.19-2.10 (m, 1H), 1.83-1.70 (m, 1H), 1.45 (t, J=6.9 Hz, 3H).

### Example 85. (S)-2-(3-((dimethylamino) methyl) pyrrolidin-1-yl)-4-ethoxy-N-(8-fluoro-2-methylimidazo[1,2-a]pyridin-6-yl)pyrimidine-5-carboxamide formate (Compound 305)

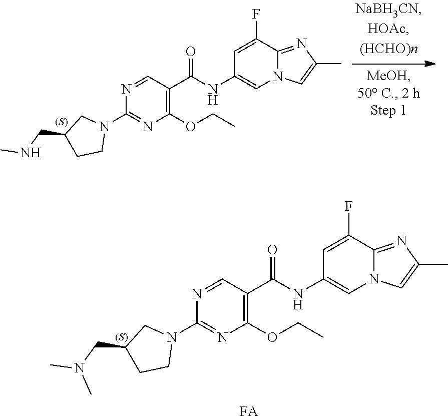

### Step 1: Preparation of(S)-2-(3-((dimethylamino) methyl) pyrrolidin-1-yl)-4-ethoxy-N-(8-fluoro-2-methylimidazo[ 1,2-a]pyridin-6-yl)pyrimidine-5-carboxamide formate

To a mixture of(S)-4-ethoxy-N-(8-fluoro-2-methylimidazo[1,2-a]pyridin-6-yl)-2-(3-((methylamino) methyl) pyrrolidin-1-yl)pyrimidine-5-carboxamide (35 mg, 0.074 mmol), AcOH (13 mg, 0.22 mmol) in MeOH (3 mL) were added NaBH₃CN (23 mg, 0.37 mmol) and (HCHO), (11 mg, 0.37 mmoL). The mixture was stirred at 50° C. for 2 h. The mixture was filtered and the filtrate was concentrated in vacuo. The residue was purified by prep-HPLC (0.1% FA in water/ACN) to give title product (11.0 mg, Y: 30.5%) as a white solid. ESI-MS (M+H⁺): 442.2. ¹H NMR (400 MHz, MeOD-d₄) δ 8.92 (s, 1H), 8.69 (s, 1H), 8.51 (s, 1H), 7.66 (d, J=2.6 Hz, 1H), 7.00 (d, J=11.8 Hz, 1H), 4.60 (q, J=6.9 Hz, 2H), 3.93-3.83 (m, 1H), 3.72-3.70 (m, 1H), 3.49-3.46 (m, 1H), 3.31-3.28 (m, 1H), 3.24-3.14 (m, 1H), 2.96-2.94 (m, 2H), 2.74 (s, 3H), 2.67 (s, 3H), 2.40 (s, 3H), 2.24-2.23 (m, 1H), 1.83-1.70 (m, 1H), 1.54 (t. J=7.1 Hz, 3H).

### Example 86. N-(7-fluoro-2-methyl-2H-indazol-5-yl)-4-isobutoxy-2-(piperazin-1-yl)pyrimidine-5-carboxamide formate (Compound 306)

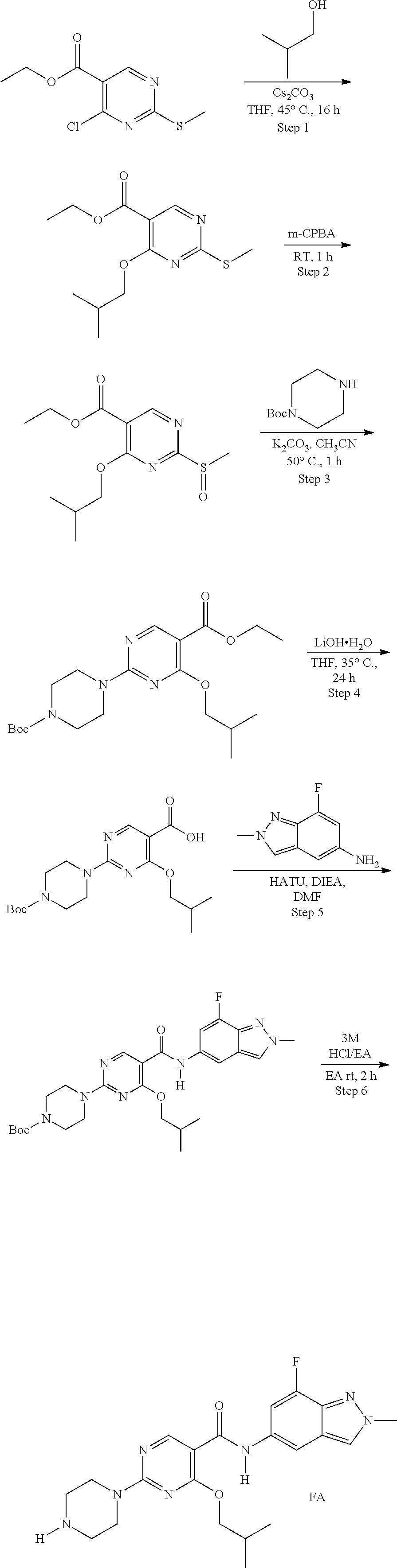

### Step 1: Preparation of ethyl 2-(methylthio)-4-propoxypyrimidine-5-carboxylate

To a solution of ethyl 4-chloro-2-(methylthio)pyrimidine-5-carboxylate (2 g, 8.6 mmol) in THF (50 mL) were added 2-methylpropan-1-ol (1.28 g, 17.24 mmol) and Cs₂CO₃ (8.43 g, 25.86 mol). The mixture was stirred at 48° C. for 16 h. The reaction mixture was diluted with water (30 mL) and extracted with EtOAc (30 mL×3). The organic layer was washed with brine, dried over Na₂SO₄ and concentrated in vacuo. The crude was purified by silica gel column chromatography (EA/PE=1:5) to give title product (4.9 g, 50%) as a colorless oil. ESI-MS (M+H)⁺: 271.4. ¹H NMR (400 MHZ, CDCl₃)δ 8.81 (s, 1H), 4.35 (q, J=7.1 Hz, 2H), 4.25 (d, J=6.5 Hz, 2H), 2.57 (s, 3H), 2.14 (dt, J=13.3, 6.7 Hz, 1H), 1.37 (t, J=7.1 Hz, 3H), 1.05 (d, J=6.7 Hz, 6H).

### Step 2: Preparation of ethyl 4-isobutoxy-2-(methylsulfinyl)pyrimidine-5-carboxylate

To a solution of ethyl 2-(methylthio)-4-propoxypyrimidine-5-carboxylate (1.1 g, 4.3 mmol) in DCM (20 mL) was added m-CPBA (1.1 g, 6.4 mmol). The mixture was stirred at rt for 1 h. The mixture was diluted with DCM and washed with water and bine, dried over Na₂SO₄ and concentrated in vacuo to give title product (1 g, 85.5%) as a white solid. ESI-MS (M+H)⁺: 287.1.

### Step 3: Preparation of ethyl 2-(4-(tert-butoxycarbonyl) piperazin-1-yl)-4-isobutoxypyrimidine-5-carboxylate

To a solution of ethyl 4-isobutoxy-2-(methylsulfinyl)pyrimidine-5-carboxylate (1 g, 3.5 mmol) in CH₃CN (30 mL) were added K₂CO₃ (0.97 g, 7 mmol) and tert-butyl piperazine-1-carboxylate (0.98 g, 5.24 mmol). The mixture was stirred at 50° C. for 1 h. The reaction mixture was diluted with water (30 mL) and extracted with EtOAc (30 mL×3). The organic layer was washed with brine, dried over Na₂SO₄ and concentrated in vacuo. The crude was purified by silica gel column chromatography (EA/PE=1:5) to give title product (1.3 g, 93%) as a colorless oil. ESI-MS (M+H)⁺: 409.2. ¹H NMR (400 MHz, CDCl₃) δ 8.74 (s, 1H), 4.30 (q, J=7.1 Hz, 2H), 4.12 (d, J=5.0 Hz, 2H), 3.85-3.84 (m, 4H), 3.58-3.47 (m, 4H), 2.16-2.07 (m, 1H), 1.49 (s, 9H), 1.35 (t, J=7.1 Hz, 3H), 1.05 (d, J=6.7 Hz, 6H).

### Step 4: Preparation of 2-(4-(tert-butoxycarbonyl) piperazin-1-yl)-4-isobutoxypyrimidine-5-carboxylic acid

To a solution of ethyl 2-(4-(tert-butoxycarbonyl) piperazin-1-yl)-4-isobutoxypyrimidine-5-carboxylate (1.3 g, 3.2 mmol) in THF: H₂O (10 mL: 10 mL) was added LiOH.H₂O) (0.27 g, 6.4 mmol). The mixture was stirred at 35° C. for 24 h. The reaction mixture was concentrated in vacuo to remove most THF. The mixture was adjusted to pH=5 with 1M HCl. The precipitate was filtered and dried in vacuo to give title product (1 g, 83%) as a white solid. ESI-MS (M+H)⁺: 381.6. ¹H NMR (400 MHZ, CDCl₃)δ 8.86 (s, 1H), 4.28 (d, J=6.6 Hz, 2H), 3.89-3.88 (m, 4H), 3.54-3.50 (m, 4H), 2.24-2.10 (m, 1H), 1.49 (s, 9H), 1.05 (d, J=6.7 Hz, 6H).

### Step 5: Preparation of tert-butyl 4-(5-((7-fluoro-2-methyl-2H-indazol-5-yl) carbamoyl)-4-isobutoxypyrimidin-2-yl) piperazine-1-carboxylate

To a solution of 2-(4-(tert-butoxycarbonyl) piperazin-1-yl)-4-isobutoxypyrimidine-5-carboxylic acid (100 mg, 0.26 mmol) in DMF (4 mL) were added 7-fluoro-2-methyl-2H-indazol-5-amine (65 mg, 0.39 mmol), HATU (150 mg, 0.39 mmol) and DIEA (135.8 mg, 1.05 mmol). The mixture was stirred at rt for 16 h. The mixture was diluted with water (20 mL), the precipitate was filtered and dried in vacuo to give title product (100 mg, 72.5%) as a white solid. ESI-MS (M+H)⁺: 528.4. ¹H NMR (400 MHZ, CDCl₃) δ 9.32 (s, 1H), 9.03 (d, J=1.6 Hz, 1H), 8.07 (s, 1H), 8.01 (s, 1H), 6.91 (d, J=11.9 Hz, 1H), 4.33 (d, J=6.4 Hz, 2H), 4.23 (s, 3H), 3.88-3.87 (m, 4H), 3.52-3.51 (m, 4H), 2.31-2.21 (m, 1H), 1.50 (s, 9H), 1.15 (d, J=6.7 Hz, 6H).

### Step 6: Preparation of N-(7-fluoro-2-methyl-2H-indazol-5-yl)-4-isobutoxy-2-(piperazin-1-yl)pyrimidine-5-carboxamide formate

To a solution of tert-butyl 4-(5-((7-fluoro-2-methyl-2H-indazol-5-yl) carbamoyl)-4-isobutoxypyrimidin-2-yl) piperazine-1-carboxylate (100 mg, 0.19 mmol) in EA (4 mL) was added 3M HCl/EA (4 mL). The mixture was stirred at rt for 2 h. The mixture was diluted with water (15 mL) and extracted with EA (20 ml×3). The aqueous phase was concentrated in vacuo. The crude was purified by prep-HPLC (0.05% FA in water/ACN) to give title product (73 mg, 81.3%) as a white solid. ESI-MS (M+H⁺): 428.1. ¹H NMR (400 MHZ, DMSO-d₆) δ 9.61 (s, 1H), 8.62 (s, 1H), 8.42 (d, J=2.7 Hz, 1H), 8.17 (d, J=5.2 Hz, 1H), 7.95 (s, 1H), 7.25 (d, J=14.2 Hz, 1H), 4.26 (d, J=6.6 Hz, 2H), 4.17 (s, 3H), 3.83-3.82 (m, 4H), 2.88-2.87 (m, 4H), 2.17 (dt, J=13.3, 6.7 Hz, 1H), 1.01 (d, J=6.7 Hz, 6H).

### Example 87. 4-ethoxy-N-(7-fluoro-2,6-dimethyl-2H-indazol-5-yl)-2-(piperazin-1-yl)pyrimidine-5-carboxamide hydrochloride (Compound 307)

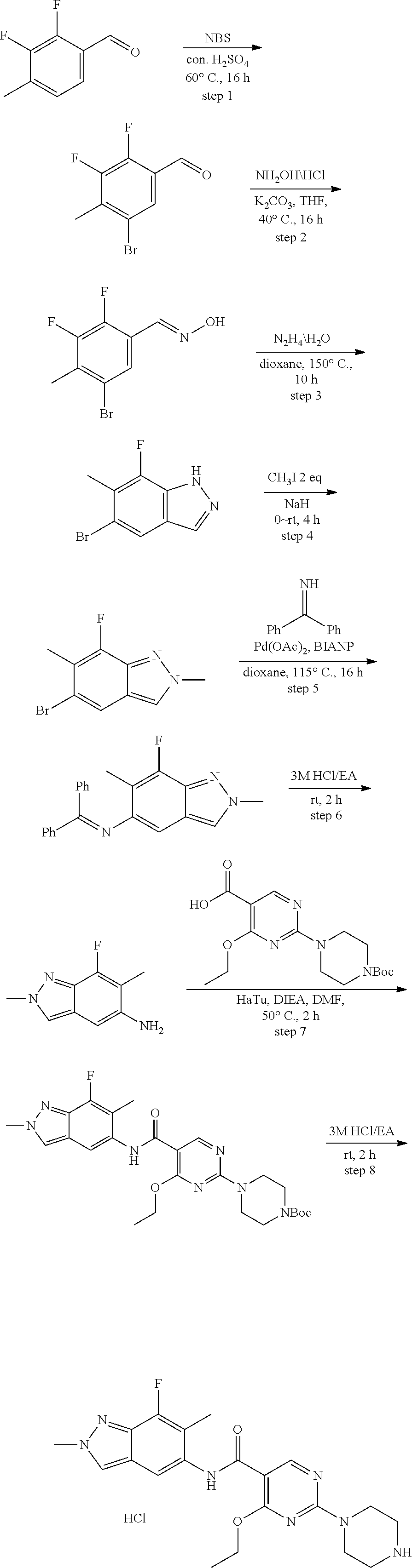

### Step 1: Preparation of 5-bromo-2,3-difluoro-4-methylbenzaldehyde

To a solution of 2,3-difluoro-4-methylbenzaldehyde (25 g, 0.16 mol) in con.H₂SO₄ (300 mL) was added NBS (31.38 g, 0.18 mmol). The mixture was stirred at 60° C. for 16 h. The mixture was poured into ice-water (500 mL) and extracted with EA (150 mL×3). The combined organics were washed with brine (300 mL×3), dried over Na₂SO₄, filtered and concentrated in vacuo. The residue was purified by column chromatography (PE) to give title product (24 g, 64.10%) as a yellow oil. ¹H NMR (400 MHZ, DMSO-d₆) δ 10.08 (s, 1H), 7.83 (dd, J=5.8, 1.8 Hz, 1H), 2.37 (d, J=2.8 Hz, 3H).

### Step 2: Preparation of (E)-5-bromo-2,3-difluoro-4-methylbenzaldehyde oxime

To a mixture of 5-bromo-2,3-difluoro-4-methylbenzaldehyde (24 g, 0.10 mol) and NH₂OH.HCl (8.58 g, 0.12 mol) in THF (300 mL) was added K₂CO₃ (16.56 g, 0.12 mol). The mixture was stirred at 40° C. for 16 h. The mixture was filtered and the filtrate was concentrated and the residue was diluted with EA (300 mL). The organic layer was washed with brine (500 mL), dried over Na₂SO₄, filtered and concentrated in vacuo to give title product (20 g, 80.32%) as a yellow oil. ESI-MS (M+H⁺): 293.0. ¹H NMR (400 MHZ, DMSO-d₆)δ 11.89 (s, 1H), 8.17 (s, 1H), 7.72 (dd, J=6.0, 1.8 Hz, 1H), 2.32 (d, J=2.6 Hz, 3H).

### Step 3: Preparation of 5-bromo-7-fluoro-6-methyl-1H-indazole

To a mixture of (E)-5-bromo-2,3-difluoro-4-methylbenzaldehyde oxime (20 g, 0.08 mol) in dioxane (200 mL) was added N₂H₄.H₂ ( ) (20.48 g, 0.64 mol). The mixture was stirred at 150° C. for 10 h. The mixture was diluted with H₂O (300 mL) and extracted with EA (300 mL×3). The organic phase was washed with brine (300 mL×3), dried over anhydrous Na₂SO₄ and concentrated. The residue was purified by column chromatography (SiO₂, Petroleum ether/Ethyl acetate=100/1 to 1/1) to afford title product (15.1 g, 82.79%) as a white solid. ESI-MS (M+H⁺): 230.9. ¹H NMR (400 MHZ, DMSO-d₆) δ 8.11 (d, J=3.4 Hz, 1H), 7.91 (s, 1H), 2.38 (d, J=2.7 Hz, 3H).

### Step 4: Preparation of 5-bromo-7-fluoro-2,6-dimethyl-2H-indazole

To a mixture of 5-bromo-7-fluoro-6-methyl-1H-indazole (15.1 g, 0.07 mol) in THF (200 mL) was added NaH(S.6 g, 0.14 mol) at 0° C., the mixture was stirred at this temperature for 30 min. CH₃I (29.82 g, 0.21 mol) was added to the mixture and stirred at rt for 4 h. The mixture was diluted with H₂O (100 mL) and extracted with EA (200 mL×2). The organic phase was washed with brine (200 mL×3), dried over anhydrous Na₂SO₄ and concentrated. The residue was purified by column chromatography (SiO₂, Petroleum ether/Ethyl acetate=100/1 to 1/1) to afford title product (7.4 g, 47.04%) as a white solid. ESI-MS (M+H⁺): 245.0. ¹H NMR (400 MHz, DMSO-d₆) δ 8.40 (d, J=2.8 Hz, 1H), 7.88 (s, 1H), 4.18 (s, 3H), 2.34 (d, J=3.0 Hz, 3H).

### Step 5: Preparation of N-(7-fluoro-2,6-dimethyl-2H-indazol-5-yl)-1,1-diphenylmethanimine

To a mixture of 5-bromo-7-fluoro-2,6-dimethyl-2H-indazole (3 g, 12.3 mmol), diphenylmethanimine (2.69 g, 14.88 mmol) in dioxane (50 mL) were added Pd₂ (dba)₃ (1.12 g, 1.23 mmol), BIANP (1.53 g, 2.46 mmol) and Cs₂CO₃ (8.02 g, 24.6 mmol), the mixture was charged with Ar for three times and stirred at 115° C. for 16 h. The mixture was diluted with H₂O) (50 mL) and extracted with EA (50 mL×3). The organic phase was washed with brine (50 mL×3), dried over anhydrous Na₂SO₄ and concentrated. The residue was purified by column chromatography (SiO₂, Petroleum ether/Ethyl acetate=100/1 to 4/1) to afford title product (3.5 g, 82.96%) as a yellow solid. ESI-MS (M+H⁺): 344.1. ¹H NMR (400 MHZ, DMSO-ď6)δ 8.07 (s, 1H), 7.70 (d, J=7.2 Hz, 2H), 7.50-7.46 (m, 4H), 7.34-7.25 (m, 2H), 7.15 (d, J=6.9 Hz, 2H), 6.40 (s, 1H), 4.05 (s, 3H), 2.17 (s, 3H)

### Step 6: Preparation of 7-fluoro-2,6-dimethyl-2H-indazol-5-amine hydrochloride

A mixture of N-(7-fluoro-2,6-dimethyl-2H-indazol-5-yl)-1,1-diphenylmethanimine (3.5 g, 10.20 mmol) in EA/HCl (50 mL) was stirred at rt for 2 h. The precipitate was filtered to give title product (1.6 g, 72%) as a yellow solid. ESI-MS (M+H⁺): 180.0. ¹H NMR (400 MHz, DMSO-d₆) ¿ 10.57 (s, 2H), 8.55 (d, J=2.4 Hz, 1H), 7.78 (s, 1H), 4.20 (s, 3H), 2.36 (s, 3H).

### Step 7: Preparation of tert-butyl 4-(4-ethoxy-5-(7-fluoro-2,6-dimethyl-2H-indazol-5-yl) carbamoyl)pyrimidin-2-yl) piperazine-1-carboxylate

To a mixture of 7-fluoro-2,6-dimethyl-2H-indazol-5-amine hydrochloride (242 mg, 1.12 mmol), 2-(4-(tert-butoxycarbonyl) piperazin-1-yl)-4-ethoxypyrimidine-5-carboxylic acid (394.24 mg, 1.12 mmol) in DMF (10 mL) were added HATU (424 mg, 1.68 mmol) and DIEA (384 mg, 4.48 mmol). The mixture was stirred at 50° C. for 2 h. Water (20 mL) was added to the mixture, the precipitate was filtered and dried under vacuum to give title product (60 mg, 11%) as a white solid ESI-MS (M+H⁺): 514.4. ¹H NMR (400 MHZ, DMSO-d₆) δ 9.26 (s, 1H), 8.76 (s, 1H), 8.38 (d, J=2.6 Hz, 1H), 8. 11 (s, 1H), 4.61 (q. J=6.7 Hz, 2H), 4.16 (s, 3H), 3.82 (d, J=5.1 Hz, 4H), 3.58 (br s, 4H), 2.30 (d, J=2.1 Hz, 3H), 1.44-1.42 (m, 12H).

### Step 8: Preparation of 4-ethoxy-N-(7-fluoro-2,6-dimethyl-2H-indazol-5-yl)-2-(piperazin-1-yl)pyrimidine-5-carboxamide hydrochloride

A mixture of tert-butyl 4-(4-ethoxy-5-((7-fluoro-2,6-dimethyl-2H-indazol-5-yl) carbamoyl)pyrimidin-2-yl) piperazine-1-carboxylate (60 mg, 0.15 mmol) in EA/HCl (50 mL) was stirred at rt for 2 h. The mixture was concentrated and lyophilized to give title product (43 mg, 69.24%) as a yellow solid. ESI-MS (M+H⁺): 414.2. ¹H NMR (400 MHZ, DMSO-d₆) δ 9.29 (s, 1H), 9.07 (s, 2H), 8.79 (s, 1H), 8.39 (d, J=2.8 Hz, 1H), 8.09 (s, 1H), 4.62 (q, J=7.0 Hz, 2H), 4.17 (s, 3H), 4.05 (br s, 4H), 3.21 (d, J=4.8 Hz, 4H), 2.30 (d, J=2.2 Hz, 3H), 1.46 (t, J=7.0 Hz, 3H).

### Example 88. 4-ethoxy-N-(7-fluoro-2-methylimidazo[1,2-a]pyridin-6-yl)-2-(piperazin-1-yl)pyrimidine-5-carboxamide formate (Compound 308)

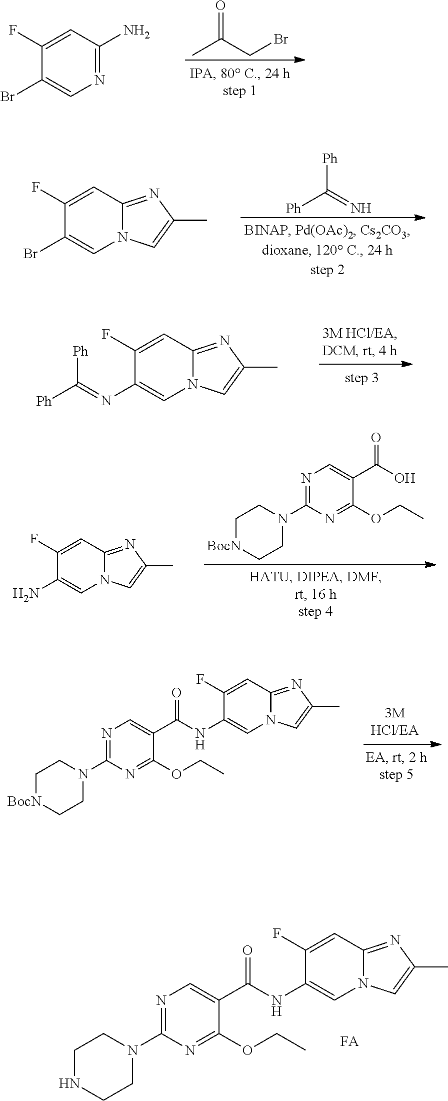

### Step 1: preparation of 6-bromo-7-fluoro-2-methylimidazo[1,2-a]pyridine

To a solution of 5-bromo-4-fluoropyridin-2-amine (10 g, 54.36 mmol) in IPA (100 mL) was added 1-bromopropan-2-one (8.61 g, 62.83 mmol). The mixture was stirred at 80° C. for 24 h. The mixture was concentrated in vacuo. The residue was diluted with 2M NaOH (50 mL) and stirred for 30 min. The precipitate was filtered and purified by flash chromatography (PE: EA=10:1) to give title product (6.7 g, Y: 53.82%) as a yellow solid. ESI-MS (M+H)⁺: 231.1. ¹H NMR (400 MHz, DMSO-d₆) δ 8.97 (d, J=7.0 Hz, 1H), 7.63 (s, 1H), 7.51 (d, J=9.8 Hz, 1H), 2.31 (s, 3H).

### Step 2: preparation of N-(7-fluoro-2-methylimidazo[1,2-a]pyridin-6-yl)-1,1-diphenylmethanimine

To a solution of 6-bromo-7-fluoro-2-methylimidazo[1,2-a]pyridine (5.50 g, 24.02 mmol) and diphenylmethanimine (5.38 g, 30 mmol) in 1,4-dioxane (55 mL) were added BINAP (2.99 g, 4.80 mmol), Pd(OAc)₂ (0.54 g, 2.40 mmol) and Cs₂CO₃ (15.66 g, 48.03 mmol). The mixture was stirred at 120° C. for 24 h. The mixture was concentrated in vacuo, the residue was purified by flash chromatography (PE:EA=10:1) to give title product (3.60 g. Y: 45.42%) as a yellow solid. ESI-MS (M+H)⁺: 330.1. ¹H NMR (400 MHz, DMSO-d₆) δ 8.00 (d, J=7.7 Hz, 1H), 7.72-7.68 (m, 2H), 7.59-7.58 (m, 1H), 7.50-7.48 (m, 2H), 7.45 (s. 1H), 7.40-7.36 (m, 3H), 7.23-7.19 (m, 3H), 2.23 (s, 3H).

### Step 3: preparation of 7-fluoro-2-methylimidazo[1,2-a]pyridin-6-amine

To a solution of N-(7-fluoro-2-methylimidazo[1,2-a]pyridin-6-yl)-1,1-diphenylmethanimine (3.60 g, 10.90 mmol) in DCM (10 mL) was added 3M HCl/EA (5 mL). The mixture was stirred at rt for 4 h. The mixture was adjusted to pH=8-9 with aqueous sodium bicarbonate and extracted with DCM. The organic phase was dried over sodium sulfate and concentrated. The residue was purified by flash chromatography (PE:EA=1:3) to get title product (1.00 g, Y: 55.60%) as a yellow solid. ESI-MS (M+H)⁺: 166.1. ¹H NMR (400 MHZ, DMSO-d₆) δ 7.79 (d, J=8.4 Hz, 1H), 7.45 (s, 1H), 7.16 (d, J=11.7 Hz, 1H), 4.87 (s, 2H), 2.23 (s, 3H).

### Step 4: preparation of tert-butyl 4-(4-ethoxy-5-((7-fluoro-2-methylimidazo[1,2-a]pyridin-6-yl) carbamoyl)pyrimidin-2-yl) piperazine-1-carboxylate

To a solution of 7-fluoro-2-methylimidazo[1,2-a]pyridin-6-amine (100 mg, 0.61 mmol) in DMF (5 mL) were added 2-(4-(tert-butoxycarbonyl) piperazin-1-yl)-4-ethoxypyrimidine-5-carboxylic acid (213 mg, 0.61 mmol), HATU (230 mg, 0.61 mmol) and DIEA (260 mg, 2.02 mmol). The mixture was stirred at rt for 16 h. The mixture was diluted with water and the precipitate was filtered and dried under vacuum to give title product (240 mg, Y: 78.69%) as a yellow solid. ESI-MS (M+H)⁺: 500.4.

### Step 5: preparation of 4-ethoxy-N-(7-fluoro-2-methylimidazo[1,2-a]pyridin-6-yl)-2-(piperazin-1-yl)pyrimidine-5-carboxamide formate

To a solution of tert-butyl 4-(4-ethoxy-5-((7-fluoro-2-methylimidazo[1,2-a]pyridin-6-yl) carbamoyl)pyrimidin-2-yl) piperazine-1-carboxylate (240 mg, 0.48 mmol) in EA (5 mL) was added 3M HCl/EA (3 mL). The mixture was stirred at rt for 2 h. The mixture was concentrated in vacuo and the residue was purified by prep-HPLC (0.05% FA in water/MeCN) to give title product (45 mg. Y: 28.1%) as a white solid. ESI-MS (M+H)⁺: 400.2. 1H NMR (400 MHZ, MeOD-d₄)δ 9.51 (d, J=6.8 Hz, 1H), 8.90 (s, 1H), 8.18 (s, 1H), 7.65 (s, 1H), 7.37 (d, J=10.9 Hz, 1H), 4.64 (q, J=6.9 Hz, 2H), 4.17 (br s, 4H), 3.32 (m, 4H), 2.40 (s, 3H), 1.57 (t, J=7.0 Hz, 3H).

### Example 89. rel-2-[(3R)-3-(2,2-difluoroethyl) piperazin-1-yl]-4-ethoxy-N-{8-fluoro-2-methylimidazo[1,2-a]pyridin-6-yl}pyrimidine-5-carboxamide and rel-2-[(3S)-3-(2,2-difluoroethyl) piperazin-1-yl]-4-ethoxy-N-{8-fluoro-2-methylimidazo[1,2-a]pyridin-6-yl}pyrimidine-5-carboxamide (Compound 309 and Compound 310

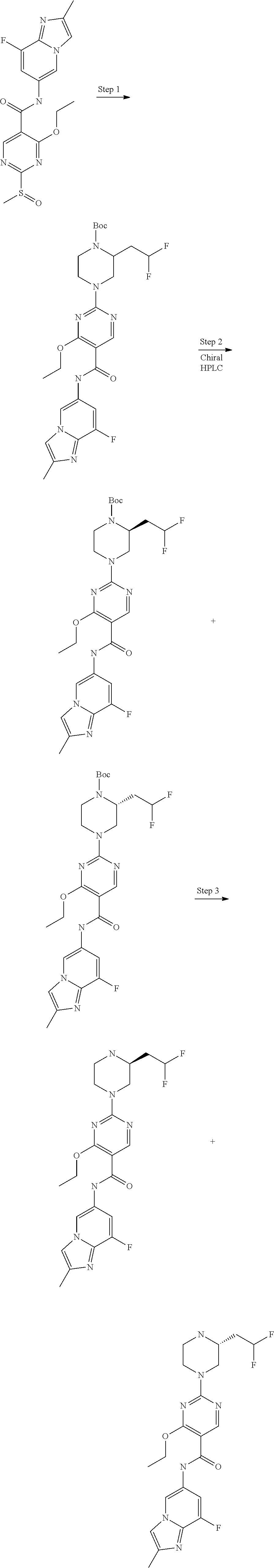 [SMILES: CCOc1nc(S(C)=O)ncc1C(=O)Nc1cc(F)c2nc(C)cn2c1 ; CCOc1nc(N2CCN(C(=O)OC(C)(C)C)C(CC(F)F)C2)ncc1C(=O)Nc1cc(F)c2nc(C)cn2c1 ; CCOc1nc(N2CCN(C(=O)OC(C)(C)C)[C@@H](CC(F)F)C2)ncc1C(=O)Nc1cc(F)c2nc(C)cn2c1 ; CCOc1nc(N2CCN(C(=O)OC(C)(C)C)[C@H](CC(F)F)C2)ncc1C(=O)Nc1cc(F)c2nc(C)cn2c1 ; CCOc1nc(N2CCN[C@@H](CC(F)F)C2)ncc1C(=O)Nc1cc(F)c2nc(C)cn2c1 ; CCOc1nc(N2CCN[C@H](CC(F)F)C2)ncc1C(=O)Nc1cc(F)c2nc(C)cn2c1]

### Step 1: Preparation of tert-butyl 2-(2,2-difluoroethyl)-4-[4-ethoxy-5-(8-fluoro-2-methylimidazo[1,2-a]pyridin-6-ylcarbamoyl)pyrimidin-2-yl]piperazine-1-carboxylate

tert-Butyl 2-(2,2-difluoroethyl) piperazine-1-carboxylate (165.65 mg, 661.85 μmol) was added dropwise to a cooled to 0° C. suspension of 4-ethoxy-N-8-fluoro-2-methylimidazo[1,2-a]pyridin-6-yl-2-methanesulfinylpyrimidine-5-carboxamide (249.78 mg, 661.85 μmol) and potassium carbonate (182.94 mg, 1.32 mmol) in MeCN (5 mL), and the reaction mass was stirred overnight at room temperature. The obtained mixture was filtered and MeCN was evaporated under reduced pressure. The residue was partitioned between EtOAc (10 mL) and H₂O (3 mL). The organic layer was separated and dried over anhydrous NazSO4, filtered, and concentrated in vacuo. The residue was purified by HPLC to give tert-butyl 2-(2,2-difluoroethyl)-4-[4-ethoxy-5-(8-fluoro-2-methylimidazo[1,2-a]pyridin-6-ylcarbamoyl)pyrimidin-2-yl]piperazine-1-carboxylate (141 mg, Y: 38%). ESI-MS (M+H)⁺: 564.2.

### Step 2: Chiral HPLC

Column: Chiralpak IB (250×20 mm, 5 mkm)

Mobile Phase: CO₂: MeOH, 80:20

Flow Rate: 50 mL/min

ESI-MS (M+H)⁺: 564.0.

RetTime (enantiomer A)=10.90 min; RetTime (enantiomer B)=9.34 min.

### Step 3: rel-2-[(3R)-3-(2,2-difluoroethyl) piperazin-1-yl]-4-ethoxy-N-{8-fluoro-2-methylimidazo[1,2-a]pyridin-6-yl}pyrimidine-5-carboxamide and rel-2-{(3S)-3-(2,2-difluoroethyl) piperazin-1-yl}-4-ethoxy-N-{8-fluoro-2-methylimidazo[1,2-a]pyridin-6-yl}pyrimidine-5-carboxamide

To a solution of rel-tert-butyl(2R)-2-(2,2-difluoroethyl)-4-[4-ethoxy-5-({8-fluoro-2-methylimidazo[1,2-a]pyridin-6-yl}carbamoyl)pyrimidin-2-yl]piperazine-1-carboxylate (47.0 mg, 83.41 μmol) in MeOH (1 mL), dioxane/HCl (0.5 mL, 10%) was slowly added dropwise at room temperature, followed by stirring the resulting mixture for 16 h. Then the mixture was concentrated in vacuo and the residue was purified by HPLC to afford rel-2-[(3R)-3-(2,2-difluoroethyl) piperazin-1-yl]-4-ethoxy-N-{8-fluoro-2-methylimidazo[1,2-a]pyridin-6-yl}pyrimidine-5-carboxamide hydrochloride (19.0 mg, Y: 44%). ESI-MS (M+H)⁺: 464.1. ¹H NMR (400 MHZ, D₂O) δ 8.83 (s, 1H), 8.51 (s, 1H), 7.68 (s, 1H), 7.38 (d, J=10.9 Hz, 1H), 6.1 (t, J=55 Hz 1H), 4.44 (q. J=7.0 Hz, 2H), 3.67-3.57 (m, 1H), 3.45-3.05 (m, 4H), 3.34-3.03 (m, 3H), 2.32 (s, 3H), 2.31-2.19 (m, 2H), 1.34 (t, J=7.1 Hz, 3H).

To a solution of rel-tert-butyl(2S)-2-(2,2-difluoroethyl)-4-[4-ethoxy-5-({8-fluoro-2-methylimidazo[1,2-a]pyridin-6-yl}carbamoyl)pyrimidin-2-yl]piperazine-1-carboxylate (56.0 mg, 99.38 μmol) in MeOH (1 mL), dioxane/HCl (0.5 mL, 10%) was slowly added dropwise at room temperature, followed by stirring the resulting mixture for 16 h. Then the mixture was concentrated in vacuo and the residue was purified by HPLC to afford rel-2-[(3S)-3-(2,2-difluoroethyl) piperazin-1-yl]-4-ethoxy-N-{8-fluoro-2-methylimidazo[1,2-a]pyridin-6-yl}pyrimidine-5-carboxamide hydrochloride (22.0 mg, Y: 51%). ESI-MS (M+H)⁺: 464.1. ¹H NMR (400 MHZ, D₂O)δ 8.83 (s, 1H), 8.51 (s, 1H), 7.68 (s, 1H), 7.38 (d, J=10.9 Hz, 1H), 6.1 (t. J=55 Hz 1H), 4.44 (q, J=7.0 Hz, 2H), 3.67-3.57 (m, 1H), 3.45-3.05 (m, 4H), 3.34-3.03 (m, 3H), 2.32 (s, 3H), 2.31-2.19 (m, 2H), 1.34 (t, J=7.1 Hz, 3H).

### Example 90. rel-4-ethoxy-N-{8-fluoro-2-methylimidazo[1,2-a]pyridin-6-yl}-2-[(3R)-3-(methylamino) pyrrolidin-1-yl]pyrimidine-5-carboxamide hydrochloride and rel-4-ethoxy-N-{8-fluoro-2-methylimidazo[1,2-a]pyridin-6-yl}-2-[(3S)-3-(methylamino) pyrrolidin-1-yl]pyrimidine-5-carboxamide hydrochloride (Compound 311 and Compound 312)

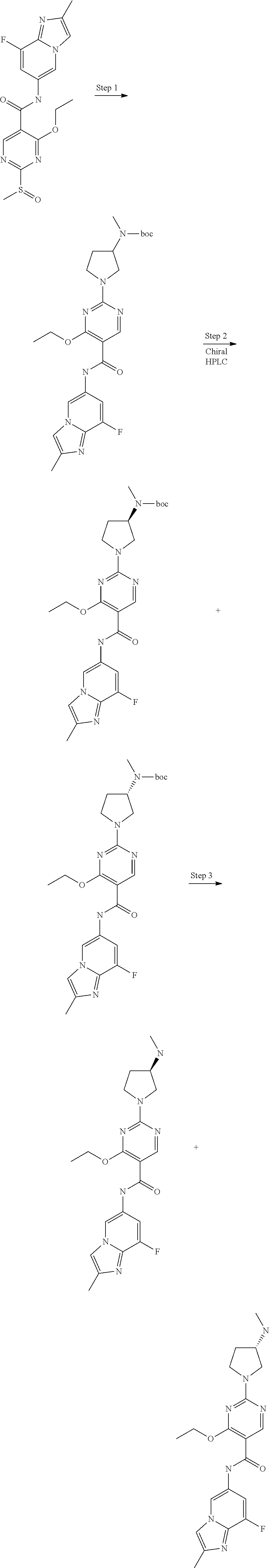

### Step 1: Preparation of tert-butyl N-1-[4-ethoxy-5-(8-fluoro-2-methylimidazo {1,2-a]pyridin-6-yl-carbamoyl)pyrimidin-2-yl|pyrrolidin-3-yl-N-methylcarbamate

tert-Butyl N-methyl-N-(pyrrolidin-3-yl) carbamate (79.56 mg, 397.22 μmol) was added dropwise to a cooled to 0° C. suspension of 4-ethoxy-N-8-fluoro-2-methylimidazo[1,2-a]pyridin-6-yl-2-methane-sulfinylpyrimidine-5-carboxamide (149.91 mg, 397.22 μmol) and potassium carbonate (109.8 mg, 794.45 μmol) in MeCN (5 mL), and the reaction mass was stirred overnight at room temperature. The obtained mixture was filtered and MeCN evaporated under reduced pressure. The residue was partitioned between EtOAc (10 ml) and H₂O (3 mL). The organic layer was separated and dried over anhydrous Na₂SO₄, filtered, and concentrated in vacuo. The residue was purified by crystallization from MTBE to give tert-butyl N-1-[4-ethoxy-5-(8-fluoro-2-methylimidazo[1,2-a]pyridin-6-ylcarbamoyl)pyrimidin-2-yl]pyrrolidin-3-yl-N-methylcarbamate (134.0 mg, Y: 64.9%). ESI-MS (M+H)⁺: 514.4.

### Step 2: Chiral HPLC

Chiral HPLC

Column: CHIRALPAK IA (250×30 mm, 5 mkm);

Mobile Phase: Hexane: IPA: MeOH, 50:25:25.

Flow Rate: 30 mL/min;

Column Temperature: 24° C.;

Wavelength: 210 nm, 215 nm.

RetTime (enantiomer A)=11.1 min; RetTime (enantiomer B)=12.5 min

### Step 3; Preparation of rel-4-ethoxy-N-(8-fluoro-2-methylimidazo[1,2-a]pyridin-6-yl}-2-[(3R)-3-(methylamino) pyrrolidin-1-yl|pyrimidine-5-carboxamide hydrochloride

To a solution of tert-butyl N-[(3R)-1-[4-ethoxy-5-(8-fluoro-2-methylimidazo[1,2-a]pyridin-6-ylcarbamoyl)-pyrimidin-2-y]]pyrrolidin-3-yl]-N-methylcarbamate (40.0 mg, 77.89 μmol) in MeOH (1 mL), dioxane/HCl (0.5 mL. 10%) was slowly added dropwise at room temperature, followed by stirring the resulting mixture for 16 h. Then the mixture was concentrated in vacuo and the residue was purified by HPLC to afford 4-ethoxy-N-8-fluoro-2-methylimidazo[1,2-a]pyridin-6-yl-2-[(3R)-3-(methylamino) pyrrolidin-1-yl]pyrimidine-5-carboxamide hydrochloride (28.0 mg, Y: 82.1%) ESI-MS (M+H)⁺: 414.2 . . . ¹H NMR (400 MHZ, CD3OD)δ 9.45 (s, 1H), 8.73 (s, 1H), 8.14 (s, 1H), 7.97 (d, J=11.0 Hz, 1H), 4.83-4.77 (q, J=7.0 Hz), 4.23-3.74 (m, 4H), 2.83 (s, 3H), 2.72-2.63 (m, 1H), 2.58 (s, 3H), 2.57-2.41 (m, 1H), 1.57 (t, J=7.1 Hz, 3H).

### rel-4-ethoxy-N-{8-fluoro-2-methylimidazo[1,2-a]pyridin-6-yl}-2-[(3S)-3-(methylamino) pyrrolidin-1-yl]pyrimidine-5-carboxamide hydrochloride

To a solution of tert-butyl N-[(3R)-1-[4-ethoxy-5-(8-fluoro-2-methylimidazo[1,2-a]pyridin-6-ylcarbamoyl)-pyrimidin-2-yl]pyrrolidin-3-yl]-N-methylcarbamate (45.0 mg, 87.62 μmol) in MeOH (1 mL), dioxane/HCl (0.5 mL, 10%) was slowly added dropwise at room temperature, followed by stirring the resulting mixture for 16 h. Then the mixture was concentrated in vacuo and the residue was purified by HPLC to afford 4-ethoxy-N-8-fluoro-2-methylimidazo[1,2-a]pyridin-6-yl-2-[(3R)-3-(methylamino) pyrrolidin-1-yl]pyrimidine-5-carboxamide hydrochloride (32.0 mg, Y: 79.2%). ESI-MS (M+H)⁺: 414.2. ¹H NMR (400 MHZ, CD₃OD)δ 9.45 (s, 1H), 8.73 (s, 1H), 8.14 (s, 1H), 7.97 (d, J=11.0 Hz, 1H), 4.83-4.77 (q, J=7.0 Hz), 4.23-3.74 (m, 4H), 2.83 (s, 3H), 2.72-2.63 (m, 1H), 2.58 (s, 3H), 2.57-2.41 (m, 1H), 1.57 (t, J=7.1 Hz, 3H).

### Example 91. 4-ethoxy-2-[(3R)-3-(methylamino) pyrrolidin-1-yl|-N-2-methylpyrazolo[1,5-a]-pyridin-5-ylpyrimidine-5-carboxamide as TosOH salt (Compound 313)

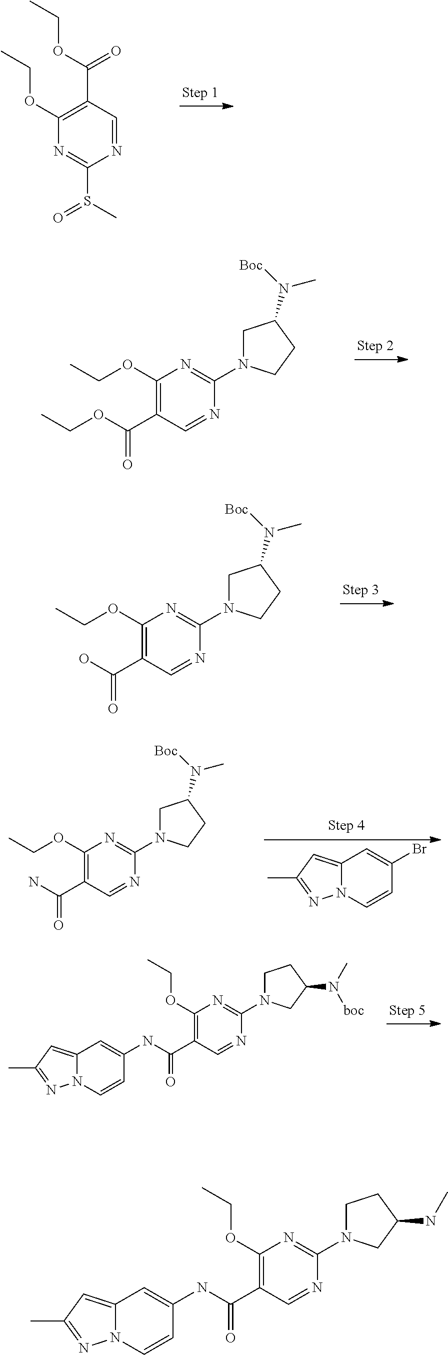

### Step 1: Preparation of ethyl 2-[(3R)-3-f (tert-butoxy) carbonyl](methyl) aminopyrrolidin-1-yl]-4-ethoxy-pyrimidine-5-carboxylate

tert-Butyl N-methyl-N-[(3R)-pyrrolidin-3-yl]carbamate hydrochloride (468.12 mg, 1.98 mmol) was added dropwise to a cooled to 0° C. suspension of ethyl 4-ethoxy-2-methanesulfinylpyrimidine-5-carboxylate (638.43 mg, 2.47 mmol) and potassium carbonate (819.85 mg, 5.93 mmol) in MeCN (5 mL), and the reaction mass was stirred overnight at room temperature. The obtained mixture was filtered and MeCN was evaporated under reduced pressure. The residue was partitioned between EtOAc (10 ml) and H₂O (3 mL). The organic layer was dried over anhydrous Na₂SO₄, filtered, and concentrated in vacuo. The residue was purified by crystallization from MTBE to give ethyl 2-[(3R)-3-[(tert-butoxy) carbonyl](methyl) aminopyrrolidin-1-yl]-4-ethoxypyrimidine-S-carboxylate (820.0 mg, Y: 94.6%). ESI-MS (M+H)⁺: 395.2.

### Step 2: Preparation of 2-| (3R)-3-{(tert-butoxy) carbonyl[(methyl) aminopyrrolidin-1-yl]-4-ethoxy-pyrimidine-5-carboxylic acid

A mixture of ethyl 2-[(3S)-3-[(tert-Butoxy) carbonyl](methyl) aminopyrrolidin-1-yl]-4-ethoxy-pyrimidine-5-carboxylate (820.0 mg, 2.08 mmol) and lithium hydroxide mono hydrate (104.72 mg, 2.5 mmol) was stirred overnight in ethanol (10 mL) and H₂O (4 mL). The reaction mixture was then concentrated under reduced pressure to remove ethanol, and the resulting aqueous solution was neutralized with 10% NaHSO₄ to pH=5 and extracted with EtOAc (2×5 mL). The organic layer was dried over Na₂SO₄ and evaporated under reduced pressure to give pure 2-[(3R)-3-[(tert-butoxy) carbonyl](methyl) aminopyrrolidin-1-yl]-4-ethoxypyrimidine-5-carboxylic acid (580.0 mg, Y: 68.5%). ESI-MS (M+H)⁺: 367.2.

### Step 3: Preparation of tert-butyl N-[(3R)-1-(5-carbamoyl-4-ethoxypyrimidin-2-yl) pyrrolidin-3-yl]-N-methylcarbamate

2-[(3S)-3-[(tert-Butoxy) carbonyl](methyl) aminopyrrolidin-1-yl]-4-ethoxypyrimidine-5-carboxylic acid (579.56 mg, 1.58 mmol) was suspended in MeCN (5 mL) and HATU (721.7 mg, 1.9 mmol) was added followed by DIPEA (408.85 mg, 3.16 mmol). The resulting mixture was stirred for 30 min at room temperature. After that ammonium hydroxide (266.07 mg. 7.59 mmol) was added in one portion and the reaction mixture was stirred overnight at room temperature. The precipitate formed was filtered, washed with MeCN (3 mL), MTBE (3 mL), and dried in vacuo to give pure tert-butyl N-[(3R)-1-(5-carbamoyl-4-ethoxypyrimidin-2-yl) pyrrolidin-3-yl]-N-methylcarbamate (510.0 mg, Y: 83.8%). ESI-MS (M+H)⁺: 366.2.

### Step 4: Preparation of tert-butyl N-[(3R)-1-14-ethoxy-5-(2-methylpyrazolo[1,5-a]pyridin-5-ylcarbamoyl)-pyrimidin-2-yl Jpyrrolidin-3-yl]-N-methylcarbamate

tert-Butyl N-[(3R)-1-(5-carbamoyl-4-ethoxypyrimidin-2-yl) pyrrolidin-3-yl]-N-methylcarbamate (100.28 mg. 274.43 μmol), 5-bromo-2-methylpyrazolo[1,5-a]pyridine (57.92 mg, 274.43 μmol), Pd> (dba): (12.57 mg, 13.72 μmol), dicyclohexyl[3,6-dimethoxy-2′,4′,6′-tris(propan-2-yl)-[1, l′-biphenyl]-2-yl]phosphane (14.73 mg, 27.44 μmol) and K₃PO4 (116.5 mg, 548.86 μmol) were suspended in dioxane (20.0 mL), and the suspension was stirred at 100° C. overnight. The resultant reaction mixture was cooled to room temperature and then filtered through Celite, and the filter cake was washed with ethyl acetate. The filtrate was washed with saturated brine and dried over anhydrous magnesium sulfate, and the solid was separated by filtration. The solution was concentrated under reduced pressure to obtain crude tert-butyl N-[(3R)-1-[4-ethoxy-5-(2-methylpyrazolo[1,5-a]pyridin-5-ylcarbamoyl)pyrimidin-2-yl]pyrrolidin-3-yl]-N-methylcarbamate (120.0 mg, Y: 35.3%) which was used in the next step without purification. ESI-MS (M+H)⁺: 496.2.

### Step 5: Preparation of 4-ethoxy-2-[(3R)-3-(methylamino) pyrrolidin-1-yl]-N-2-methylpyrazolo[1,5-a]-pyridin-5-ylpyrimidine-5-carboxamide as TosOH salt

tert-Butyl N-[(3R)-1-[4-ethoxy-5-(2-methylpyrazolo[1,5-a]pyridin-5-ylcarbamoyl)pyrimidin-2-yl]pyrrolidin-3-yl]-N-methylcarbamate (120.15 mg, 242.46 μmol) and 4-methylbenzene-1-sulfonic acid hydrate (55.34 mg, 290.95 μmol) in THF were heated at reflux overnight and then allowed to cool to room temperature. The solvent was removed under reduced pressure and the residue was purified by HPLC to give 4-ethoxy-2-[(3R)-3-(methylamino) pyrrolidin-1-yl]-N-2-methylpyrazolo[1,5-a]pyridin-5-ylpyrimidine-5-carboxamide; 4-methyl-benzene-1-sulfonic acid (2.9 mg, Y: 1.8%). ESI-MS (M+H)⁺: 396.2. ¹H NMR (400 MHz, CD₃OD) δ 8.77 (d, J=2.0 Hz, 1H), 8.30 (d, J=7.9 Hz, 1H), 8.04 (s, 1H), 7.67 (d, J=8.1 Hz, 2H), 7.19 (d, J=8.1 Hz, 2H), 6.84 (d, J=7.9 Hz, 1H), 6.27 (s, 1H), 4.63 (q, J=7.2 Hz, 2H), 4.06-3.63 (m, 5H), 2.78 (s, 3H), 2.54-2.42 (m, 1H), 2.39 (s, 3H), 2.33 (s, 3H), 2.27-2.15 (m, 1H), 1.54 (t, J=7.2 Hz, 3H).

### Example 92. 4-ethoxy-2-[(3S)-3-(methylamino) pyrrolidin-1-yl]-N-2-methylpyrazolo[1,5-a]-pyridin-5-ylpyrimidine-5-carboxamide as TosOH salt (Compound 314)

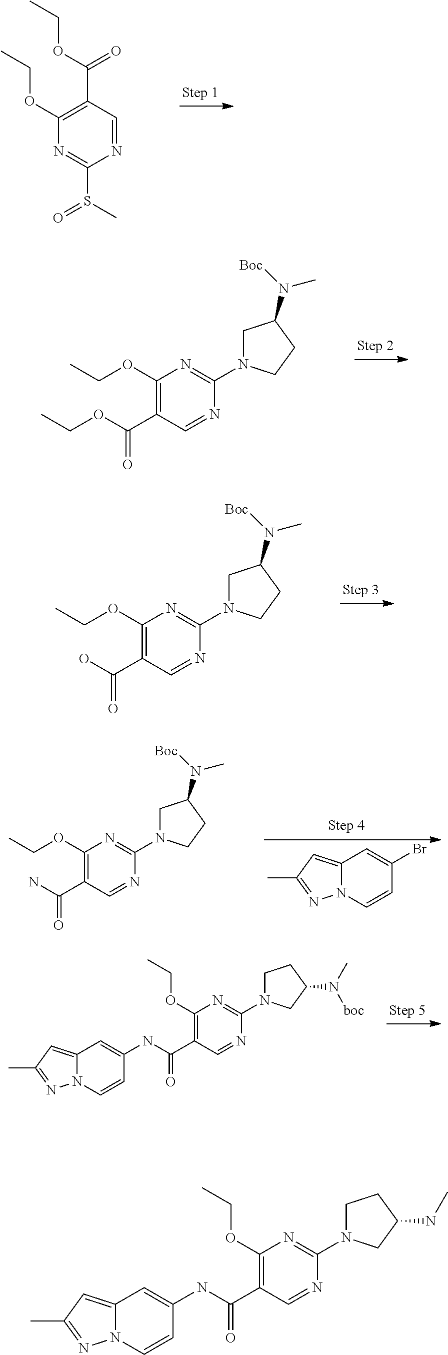

### Step 1: Preparation of ethyl 2-[(3S)-3-[(tert-butoxy) carbonyl](methyl) aminopyrrolidin-1-yl]-4-ethoxy-pyrimidine-5-carboxylate

tert-Butyl N-methyl-N-[(3S)-pyrrolidin-3-yl]carbamate (775.29 mg, 3.87 mmol) was added dropwise to a cooled to 0° C. suspension of ethyl 4-ethoxy-2-methanesulfinylpyrimidine-5-carboxylate (1.0 g, 3.87 mmol) and potassium carbonate (1.61 g, 11.61 mmol) in MeCN (5 ml . . . ), and the reaction mass was stirred overnight at room temperature. The obtained mixture was filtered and MeCN was evaporated under reduced pressure. The residue was partitioned between EtOAc (10 mL) and H₂O (3 mL). The organic layer was dried over anhydrous Na₂SO₄, filtered, and concentrated in vacuo. The residue was purified by crystallization from MTBE to give ethyl 2-[(35)-3-[(tert-butoxy) carbonyl](methyl) aminopyrrolidin-1-yl]-4-ethoxypyrimidine-5-carboxylate (1.26 g, Y: 70.1%). ESI-MS (M+H)⁺: 395.2.

### Step 2: Preparation of 2-[(3S)-3-[(tert-butoxy) carbonyl](methyl) aminopyrrolidin-1-yl]-4-ethoxy-pyrimidine-5-carboxylic acid

A mixture of ethyl 2-[(3S)-3-[(tert-Butoxy) carbonyl](methyl) aminopyrrolidin-1-yl]-4-ethoxypyrimidine-5-carboxylate (1.26 g, 3.19 mmol) and lithium hydroxide mono hydrate (160.79 mg, 3.83 mmol) was stirred overnight in ethanol (10 mL) and H₂O (4 mL). The reaction mixture was then concentrated under reduced pressure to remove ethanol, and the resulting aqueous solution was neutralized with 10% NaHSO₄ to pH=5 and extracted with EtOAc (2×5 mL). The organic layer was dried over Na₂SO₄ and evaporated under reduced pressure to give pure 2-[(35)-3-[(tert-butoxy) carbonyl](methyl) aminopyrrolidin-1-yl]-4-ethoxypyrimidine-5-carboxylic acid (500.0 mg, Y: 38.5%). ESI-MS (M+H)⁺: 367.2.

### Step 3: Preparation of tert-butyl N-[(3S)-1-(5-carbamoyl-4-ethoxypyrimidin-2-yl) pyrrolidin-3-yl]-N-methylcarbamate

2-[(3S)-3-[(tert-Butoxy) carbonyl](methyl) aminopyrrolidin-1-yl]-4-ethoxypyrimidine-5-carboxylic acid (500.34 mg, 1.37 mmol) was suspended in MeCN (5 mL) and HATU (623.06 mg, 1.64 mmol) was added followed by DIPEA (352.97 mg, 2.73 mmol). The resulting mixture was stirred for 30 min at room temperature. After that ammonium hydroxide (229.71 mg, 6.55 mmol) was added in one portion and the reaction mixture was stirred overnight at room temperature. The precipitate formed was filtered, washed with MeCN (3 mL), MTBE (3 mL), and dried in vacuo to give pure tert-butyl N-[(3S)-1-(5-carbamoyl-4-ethoxypyrimidin-2-yl) pyrrolidin-3-yl]-N-methylcarbamate (480.0 mg, Y: 86.6%). ESI-MS (M+H)⁺: 366.2.

### Step 4: Preparation of tert-butyl N-[(3R)-1-[4-ethoxy-5-(2-methylpyrazolo[1,5-a]pyridin-5-ylcarbamoyl)-pyrimidin-2-yl]pyrrolidin-3-yl]-N-methylcarbamate

tert-Butyl N-[(3S)-1-(5-carbamoyl-4-ethoxypyrimidin-2-yl) pyrrolidin-3-yl]-N-methylcarbamate (149.69 mg, 409.63 μmol), 5-bromo-2-methylpyrazolo[1,5-a]pyridine (86.46 mg, 409.63 μmol), Pd₂ (dba)₃ (18.76 mg, 20.48 μmol), dicyclohexyl[3,6-dimethoxy-2′,4′,6′-tris(propan-2-yl)-[1,1′-biphenyl]-2-yl]phosphane (21.99 mg. 40.96 μmol) and K₃PO4 (173.9 mg, 819.25 μmol) were suspended in dioxane (20.0 mL), and the suspension was stirred at 100° C. overnight. The resultant reaction mixture was cooled to room temperature and then filtered through Celite, and the filter cake was washed with ethyl acetate. The filtrate was washed with saturated brine and dried over anhydrous magnesium sulfate, and the solid was separated by filtration. The solution was concentrated under reduced pressure to obtain crude tert-butyl N-[(3R)-1-[4-ethoxy-5-(2-methylpyrazolo[1,5-a]pyridin-5-ylcarbamoyl)pyrimidin-2-yl]pyrrolidin-3-yl]-N-methylcarbamate (120.0 mg, Y: 35.3%) was used in the next step without purification. ESI-MS (M+H)⁺: 496.2.

### Step 5: Preparation of 4-ethoxy-2-[(3S)-3-(methylamino) pyrrolidin-1-yl]-N-2-methylpyrazolo[1,5-a]-pyridin-5-ylpyrimidine-5-carboxamide as TosOH salt

tert-Butyl N-[(3S)-1-[4-ethoxy-5-(2-methylpyrazolo[1,5-a]pyridin-5-ylcarbamoyl)-pyrimidin-2-yl]pyrrolidin-3-yl]-N-methylcarbamate (76.04 mg, 153.44 μmol) and 4-methylbenzene-1-sulfonic acid hydrate (58.37 mg, 306.87 μmol) in THF was heated at reflux overnight and then allowed to cool to room temperature. The solvent was removed under reduced pressure and the residue was purified by HPLC to give 4-ethoxy-2-[(3S)-3-(methylamino) pyrrolidin-1-yl]-N-2-methylpyrazolo[1,5-a]pyridin-5-ylpyrimidine-5-carboxamide; 4-methyl-benzene-1-sulfonic acid (0.007 g, Y: 8%). ESI-MS (M+H)⁺: 396.0. ¹H NMR (400 MHZ, CD₃OD) & 8.77 (d, J=2.0 Hz, 1H), 8.30 (d, J=7.9 Hz, 1H), 8.04 (s, 1H), 7.67 (d, J=8.1 Hz, 2H), 7.19 (d, J=8.1 Hz, 2H), 6.84 (d, J=7.9 Hz, 1H), 6.27 (s, 1H), 4.63 (q, J=7.2 Hz, 2H), 4.06-3.63 (m, 5H), 2.78 (s, 3H), 2.54-2.42 (m, 1H), 2.39 (s, 3H), 2.33 (s, 3H), 2.27-2.15 (m, 1H), 1.54 (t, J=7.2 Hz, 3H).

### Example 93. 2-(2,5-dihydro-1H-pyrrol-3-yl)-4-ethoxy-N-8-fluoro-2-methylimidazo[1,2-a]-pyridin-6-ylpyrimidine-5-carboxamide as TosOH salt (Compound 315)

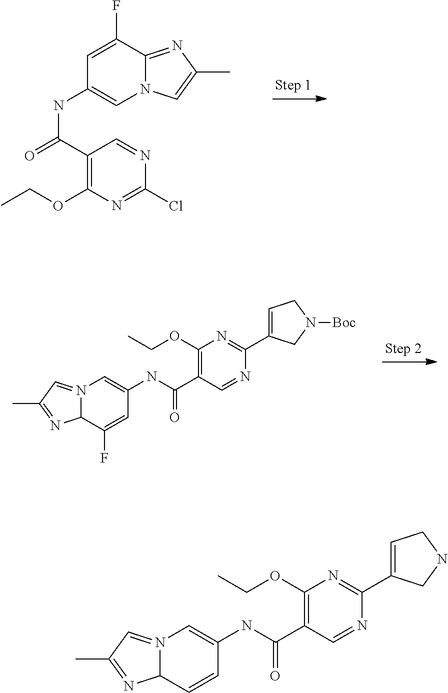

### Step 1: Preparation of tert-butyl 3-[4-ethoxy-5-(8-fluoro-2-methylimidazo[1,2-a]pyridin-6-ylcarbamoyl)pyrimidin-2-yl]-2,5-dihydro-1H-pyrrole-1-carboxylate

2-Chloro-4-ethoxy-N-8-fluoro-2-methylimidazo[1,2-a]pyridin-6-ylpyrimidine-5-carboxamide (200.06 mg, 572.01 μmol), tert-butyl 3-(tetramethyl-1,3,2-dioxaborolan-2-yl)-2,5-dihydro-1H-pyrrole-1-carboxylate (168.85 mg, 572.01 μmol), [1, l′-bis(diphenylphosphino) ferrocene]-dichloropalladium (II) dichloromethane adduct (23.36 mg, 28.6 μmol), and potassium carbonate (158.11 mg, 1.14 mmol) were suspended in degassed dioxane/water (5 mL/1 mL). The reaction mixture was heated at 90° C. overnight under an argon atmosphere. After cooling to room temperature, the mixture was diluted with ethyl acetate and the precipitate formed was filtered, washed with H₂O (1 mL) and EtOAc (2 mL), and dried in vacuo to afford tert-butyl 3-[4-ethoxy-5-(8-fluoro-2-methylimidazo[1,2-a]pyridin-6-ylcarbamoyl)pyrimidin-2-yl]-2,5-dihydro-1]-pyrrole-1-carboxylate (230.0 mg, Y: 83.3%). ESI-MS (M+H)⁺: 483.2.

### Step 2: Preparation of 2-(2,5-dihydro-1H-pyrrol-3-yl)-4-ethoxy-N-8-fluoro-2-methylimidazo[1,2-a]-pyridin-6-ylpyrimidine-5-carboxamide TosOH salt

To a solution of tert-butyl 3-[4-ethoxy-5-(8-fluoro-2-methylimidazo[1,2-a]pyridin-6-ylcarbamoyl)-pyrimidin-2-yl]-2,5-dihydro-1H-pyrrole-1-carboxylate (100.0 mg. 207.25 μmol) in DCM (10 mL), trifluoroacetic acid (458.31 mg. 2.07 mmol) was slowly added dropwise at room temperature, followed by stirring the resulting mixture for 16 h. Then the mixture was concentrated in vacuo, the residue was dissolved in MeOH (5 mL), neutralized with an aq. sat. solution of NaHCO₃, evaporated to dryness and triturated with a minimal amount of MeOH. The precipitate was filtered off and the filtrate was acidified with TosOH to pH˜3. The resulting mixture was evaporated and the residue was purified by HPLC to afford 2-(2,5-dihydro-1H-pyrrol-3-yl)-4-ethoxy-N-8-fluoro-2-methylimidazo[1,2-a]pyridin-6-ylpyrimidine-5-carboxamide TosOH salt (46.0 mg, Y: 52%). ESI-MS (M+H)⁺: 383.2. ¹H NMR (500 MHz, DMSO-d₆) δ 10.44 (s, 1H), 9.29 (s, 2H), 9.20 (s, 1H), 8.86 (s, 1H), 8.03 (s, 1H), 7.45 (d, J=7.8 Hz, 2H), 7.35 (d, J=11.8 Hz, 1H), 7.09 (d, J=7.8 Hz, 2H), 4.56 (q, J=7.0 Hz, 2H), 4.37 (s, 2H), 4.27 (s, 2H), 2.37 (s, 3H), 2.26 (s, 3H), 1.39 (t, J=7.0 Hz, 3H).

### Example 94. 4-ethoxy-N-8-fluoro-2-methylimidazo[1,2-a]pyridin-6-yl-2-(pyrrolidin-3-yl)-pyrimidine-5-carboxamide as TosOH salt (Compound 316)

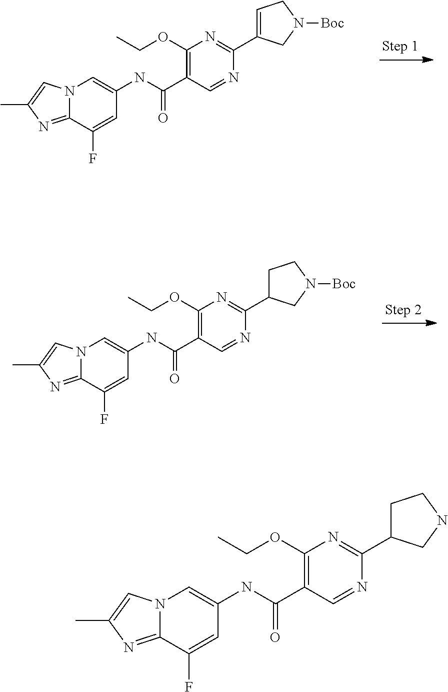 [SMILES: CCOc1nc(C2=CCN(C(=O)OC(C)(C)C)C2)ncc1C(=O)Nc1cc(F)c2nc(C)cn2c1 ; CCOc1nc(C2CCN(C(=O)OC(C)(C)C)C2)ncc1C(=O)Nc1cc(F)c2nc(C)cn2c1 ; CCOc1nc(C2CCNC2)ncc1C(=O)Nc1cc(F)c2nc(C)cn2c1]

### Step 1: Preparation of tert-butyl 3-[4-ethoxy-5-(8-fluoro-2-methylimidazo[1,2-a]pyridin-6-ylcarbamoyl)-pyrimidin-2-yl]pyrrolidine-1-carboxylate

To a stirred suspension of tert-butyl 3-[4-ethoxy-5-(8-fluoro-2-methylimidazo[1,2-a]pyridin-6-ylcarbamoyl)pyrimidin-2-yl]-2,5-dihydro-1H-pyrrole-1-carboxylate (110.0 mg, 227.98 μmol) in MeOH (5 mL) Pd/C (10 mg, 10% w) was added and the flask was degassed and purged with H₂. The resulting mixture was stirred overnight under pressure of Hz (balloon). After that catalyst was filtered off and the solvent was evaporated under reduced pressure to give crude tert-butyl 3-[4-ethoxy-5-(8-fluoro-2-methylimidazo[1,2-a]pyridin-6-ylcarbamoyl)pyrimidin-2-yl]pyrrolidine-1-carboxylate (110.0 mg, Y: 100%). ESI-MS (M+H)⁺: 485.2.

### Step 2: Preparation of 2-4-ethoxy-N-8-fluoro-2-methylimidazo[1,2-a]pyridin-6-yl-2-(pyrrolidin-3-yt)-pyrimidine-5-carboxamide TosOH salt

To a solution of tert-butyl 3-[4-ethoxy-5-(8-fluoro-2-methylimidazo[1,2-a]pyridin-6-ylcarbamoyl)pyrimidin-2-yl]pyrrolidine-1-carboxylate (109.85 mg, 226.71 μmol) in DCM (10 mL), TFA (258.5 mg, 2.27 mmol) was slowly added dropwise at room temperature, followed by stirring the resulting mixture for 16 h. Then the mixture was concentrated in vacuo, the residue was dissolved in MeOH (5 mL), neutralized with an aq. sat. solution of NaHCO₃, evaporated to dryness and triturated with minimal amount of MeOH. The precipitate was filtered, and the filtrate was acidified with TosOH to pH˜3. The resulting mixture was evaporated under reduced pressure and the residue was purified by HPLC to afford 4-ethoxy-N-8-fluoro-2-methylimidazo[1,2-a]pyridin-6-yl-2-(pyrrolidin-3-yl)pyrimidine-5-carboxamide TosOH salt (4.9 mg, Y: 3.9%). ESI-MS (M+H)⁺: 385.2. ¹H NMR (500 MHz, CD₃OD)δ 9.17 (s, 1H), 8.97 (s, 1H), 7.85 (s, 1H), 7.69 (d, J=7.8 Hz, 2H), 7.38 (d, J=11.3 Hz, 1H), 7.22 (d, J=7.8 Hz, 2H), 4.70 (q, J=7.0 Hz, 1H), 3.94-3.78 (m, 2H), 3.73-3.59 (m, 1H), 3.54-3.40 (m, 2H), 2.61-2.39 (m, 1H), 2.47 (s, 3H), 2.40-2.31 (m, 1H) 2.36 (s, 3H), 1.52 (t. J=7.1 Hz, 3H).

Biological Activity of the Compounds of the Present Disclosure Mini-gene reporter assay-PMS1

Mini-gene reporter constructs for each target site can be constructed by first PCR amplifying the region-of-interest including the sequence of the alternatively skipped, sequences of the immediate upstream and downstream introns, and sequences of the immediate upstream and downstream exons. Then the 3′end of the amplified sequence can be ligated to a firefly luciferase reporter gene and cloned into the pcDNA3.1 vector backbone. The final reporter construct can be transiently transfected into HEK293 cells using Lipofectamine 3000 transfection reagents. Compounds that can induce the inclusion of the skipped exon in the reporter construct would increase the reporter firefly luciferase activity.

Test compounds may be diluted in duplicates and dispensed into an assay plate. Transient mini-gene reporter cell line stock is resuspended and dispensed into the assay plate. Assay plates are then centrifuged and incubated. A luciferase reagent (e.g. ONE-Glo firefly luciferase reagent from Promega®) may be added to the assay plate and the luminescence (RLU) signals recorded. PMS1 EC₅₀ values may be determined by curve fitting in Levenberg-Marquardt algorithm.

In Table 2 below (second column), A indicates a IC50 (nM)<500 nM, B indicates a IC50 (nM)₅₀₀ nM to <1000 nM, C indicates a IC50 (nM)₁₀₀₀ nM to <3000 nM, D indicates a IC50 (nM)≥3000 nM. In Table 2 below (third column), A indicates a >75% Effect at 5 μM, B indicates a 50 to <75% Effect at 5 μM, and C indicates a<50% Effect at 5 μM.

TABLE 2
PMS1 Assay Data

| Compound | PMS1 IC₅₀ (nM) | PMS1% Effect at 5 μM |
|---|---|---|
| 1 | C | |
| 2 | A | |
| 3 | B | |
| 4 | B | |
| 5 | C | |
| 6 | C | |
| 7 | B | |
| 8 | A | |
| 9 | C | |
| 10 | D | |
| 11 | A | |
| 12 | C | |
| 13 | B | |
| 14 | A | |
| 15 | A | |
| 16 | D | |
| 17 | A | |
| 18 | A | |
| 19 | | B |
| 20 | A | |
| 21 | C | |
| 22 | A | |
| 23 | D | |
| 24 | | B |
| 25 | C | |
| 26 | B | |
| 27 | B | |
| 28 | C | |
| 29 | D | |
| 30 | A | |
| 31 | D | |
| 32 | C | |
| 33 | D | |
| 34 | D | |
| 35 | D | |
| 36 | A | |
| 37 | A | |
| 38 | C | |
| 39 | A | |
| 40 | C | |
| 41 | D | |
| 42 | B | |
| 43 | D | |
| 44 | C | |
| 45 | D | |
| 46 | A | |
| 47 | | C |
| 48 | D | |
| 49 | A | |
| 50 | | C |
| 51 | A | |
| 52 | D | |
| 53 | B | |
| 54 | A | |
| 55 | D | |
| 56 | B | |
| 57 | A | |
| 58 | B | |
| 59 | A | |
| 60 | B | |
| 61 | D | |
| 62 | A | |
| 63 | B | |
| 64 | A | |
| 65 | B | |
| 66 | | C |
| 67 | B | |
| 68 | D | |
| 69 | | B |
| 70 | A | |
| 71 | B | |
| 72 | B | |
| 73 | B | |
| 74 | D | |
| 75 | | C |
| 76 | A | |
| 77 | A | |
| 78 | A | |
| 79 | C | |
| 80 | A | |
| 81 | C | |
| 82 | C | |
| 83 | B | |
| 84 | A | |
| 85 | D | |
| 86 | A | |
| 87 | C | |
| 88 | A | |
| 89 | D | |
| 90 | C | |
| 91 | | C |
| 92 | C | |
| 93 | | C |
| 94 | D | |
| 95 | A | |
| 96 | C | |
| 97 | C | |
| 98 | A | |
| 99 | A | |
| 100 | D | |
| 101 | B | |
| 102 | B | |
| 103 | C | |
| 104 | D | |
| 105 | A | |
| 106 | D | |
| 107 | C | |
| 108 | B | |
| 109 | C | |
| 110 | C | |
| 111 | D | |
| 112 | C | |
| 113 | C | |
| 114 | A | |
| 115 | D | |
| 116 | A | |
| 117 | C | |
| 118 | C | |
| 119 | A | |
| 120 | A | |
| 121 | C | |
| 122 | B | |
| 123 | A | |
| 124 | D | |
| 125 | B | |
| 126 | C | |
| 127 | B | |
| 128 | A | |
| 129 | D | |
| 130 | C | |
| 131 | B | |
| 132 | A | |
| 133 | B | |
| 134 | C | |
| 135 | B | |
| 136 | A | |
| 137 | D | |
| 138 | C | |
| 139 | A | |
| 140 | | C |
| 141 | B | |
| 142 | C | |
| 143 | A | |
| 144 | D | |
| 145 | D | |
| 146 | A | |
| 147 | A | |
| 148 | C | |
| 149 | D | |
| 150 | C | |
| 151 | C | |
| 152 | B | |
| 153 | | B |
| 154 | A | |
| 155 | A | |
| 156 | C | |
| 157 | A | |
| 158 | C | |
| 159 | D | |
| 160 | A | |
| 161 | C | |
| 162 | | C |
| 163 | A | |
| 164 | A | |
| 165 | C | |
| 166 | C | |
| 167 | C | |
| 168 | A | |
| 169 | B | |
| 170 | | C |
| 171 | C | |
| 172 | | C |
| 173 | A | |
| 174 | C | |
| 175 | D | |
| 176 | B | |
| 177 | C | |
| 178 | A | |
| 179 | A | |
| 180 | A | |
| 181 | C | |
| 182 | B | |
| 183 | D | |
| 184 | B | |
| 185 | C | |
| 186 | A | |
| 187 | A | |
| 188 | C | |
| 189 | D | |
| 190 | A | |
| 191 | A | |
| 192 | B | |
| 193 | B | |
| 194 | B | |
| 195 | C | |
| 196 | | C |
| 197 | A | |
| 198 | C | |
| 199 | A | |
| 200 | C | |
| 201 | A | |
| 202 | B | |
| 203 | A | |
| 204 | B | |
| 205 | B | |
| 206 | A | |
| 207 | A | |
| 208 | A | |
| 209 | A | |
| 210 | B | |
| 211 | A | |
| 212 | A | |
| 213 | A | |
| 214 | A | |
| 215 | C | |
| 216 | A | |
| 217 | C | |
| 218 | D | |
| 219 | A | |
| 220 | A | |
| 221 | C | |
| 222 | C | |
| 223 | D | |
| 224 | C | |
| 225 | A | |
| 226 | A | |
| 227 | | C |
| 228 | B | |
| 229 | A | |
| 230 | A | |
| 231 | A | |
| 232 | B | |
| 233 | B | |
| 234 | A | |
| 235 | A | |
| 236 | A | |
| 237 | D | |
| 238 | A | |
| 239 | A | |
| 240 | A | |
| 241 | A | |
| 242 | A | |
| 243 | A | |
| 244 | A | |
| 245 | A | |
| 246 | A | |
| 247 | A | |
| 248 | A | |
| 249 | D | |
| 250 | D | |
| 251 | C | |
| 252 | D | |
| 253 | A | |
| 254 | D | |
| 255 | D | |
| 256 | A | |
| 257 | B | |
| 258 | A | |
| 259 | C | |
| 260 | A | |
| 261 | A | |
| 262 | A | |
| 263 | A | |
| 264 | C | |
| 265 | D | |
| 266 | C | |
| 267 | B | |
| 268 | C | |
| 269 | C | |
| 270 | A | |
| 271 | D | |
| 272 | C | |
| 273 | C | |
| 274 | D | |
| 275 | A | |
| 276 | D | |
| 277 | A | |
| 278 | A | |
| 279 | A | |
| 280 | C | |
| 281 | | A |
| 282 | A | |
| 283 | A | |
| 284 | A | |
| 285 | C | |
| 286 | B | |
| 287 | C | |
| 288 | B | |
| 289 | C | |
| 290 | C | |
| 291 | D | |
| 292 | C | |
| 293 | D | |
| 294 | D | |
| 295 | B | |
| 296 | D | |
| 297 | A | |
| 298 | C | |
| 299 | D | |
| 300 | C | |
| 301 | C | |
| 302 | D | |
| 303 | B | |
| 304 | B | |
| 305 | B | |
| 306 | C | |
| 307 | C | |
| 308 | | A |
| 309 | | B |
| 310 | | B |
| 311 | | A |
| 312 | A | |
| 313 | | A |
| 314 | | A |
| 315 | | A |
| 316 | | B |
| 317 | D | |
| 318 | D | |
| 319 | D | |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I): 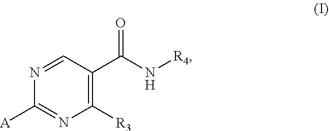
or a pharmaceutically acceptable salt, solvate, or prodrug thereof, wherein:
A is saturated or partially unsaturated mono-, bi-, or tri-cyclic 4- to 14-membered heterocycloalkyl or NR₁R₂, wherein the heterocycloalkyl comprises 1 or 2 nitrogen ring atoms and is optionally substituted with 1, 2, 3, or 4 R₆;
R₁ is —(CH₂)₀₋₂-heterocycloalkyl of 4-14 ring atoms comprising at least one nitrogen ring atom and 0-2 additional ring heteroatoms independently selected from N, O, and S; R₁ optionally substituted with 1, 2, 3, or 4 R₆;
R₂ is hydrogen, C₁₋₇alkyl, or C₃₋₈cycloalkyl;
Rₐ is halo, C₁₋₇alkyl, OR₅, N(R₅)₂, C₃₋₈cycloalkyl, or heterocycloalkyl;
R₄ is aryl, or bicyclic 9-membered heteroaryl comprising 2, 3, or 4 heteroatoms independently selected from N, O, and S, wherein Rₐ is optionally substituted with 1, 2, or 3 R₁;
each R₅ is independently C₁₋₇alkyl, C₁₋₇haloalkyl, C₃₋₈cycloalkyl, or heterocycloalkyl;
each R₆ is independently hydroxy, halogen, C₁₋₇alkyl, —(CH₂)₀₋₃—NRₐR_{b}, C₁₋₇heteroalkyl, —(CH₂)₀₋₃—C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen, OH, or C₁₋₇alkoxy, and cycloalkyl and heterocycloalkyl are optionally substituted with 1-4 independently selected C₁₋₇alkyl, halogen, or OH;
or two R₆ together form oxo (═O);
or two R₆ together form C₁₋₇alkylene to form a ring;
each R′ is hydrogen or C₁₋₇alkyl;
each Ry is independently halo, cyano, C₁₋₇alkyl, C₁₋₇haloalkyl, C₁₋₇alkoxy, C₁₋₇ haloalkoxy, or C₃₋₈cycloalkyl, wherein the C₁₋₇alkyl is optionally substituted with OH;
each Rₐ is independently H, C₁₋₇alkyl, C₁₋₇haloalkyl, or C₃₋₈cycloalkyl; and
each R_{b} is independently H, C₁₋₇alkyl, C₁₋₇haloalkyl, or C₃₋₈cycloalkyl.

2. A compound of Formula (I): 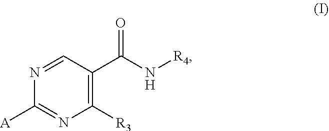
or a pharmaceutically acceptable salt, solvate, or prodrug thereof, wherein:
A is saturated or partially unsaturated mono- or bi-cyclic 4- to 11-membered nitrogen-containing heterocycloalkyl or NR: R₂, wherein the nitrogen-containing heterocycloalkyl comprises 1 or 2 nitrogen ring atoms and is optionally substituted with 1, 2, 3, or 4 R₆;
R₁ is heterocycloalkyl comprising 1 nitrogen ring atom, optionally substituted with 1, 2, 3, or 4 R₆;
R₇ is hydrogen, C₁₋₇alkyl, or C₃₋₈cycloalkyl;
R₃ is C₁₋₇alkyl, OR₅, N(R₅)₂, C₃₋₈cycloalkyl, or heterocycloalkyl;
R₄ is a bicyclic 9-membered heteroaryl comprising 2, 3, or 4 heteroatoms independently selected from N and O), wherein R₄ can be optionally substituted with 1, 2, or 3 R₇;
each R₅ is independently C₁₋₇alkyl, C₁₋₇haloalkyl, C₃₋₈cycloalkyl, or heterocycloalkyl;
each R₆ is independently hydroxy, halogen, C₁₋₇alkyl, —(CH₂)₀₋₃—NRₐR_{b}, C₁₋₇heteroalkyl, —(CH₂)₆₋₃—C₃₋₈cycloalkyl, —NR′—(CH₂)₀₋₃—C₃₋₈cycloalkyl, —(CH₂)₀₋₃-heterocycloalkyl, —C(O)-heterocycloalkyl, or —O-heterocycloalkyl, wherein alkyl is optionally substituted with 1-4 independently selected halogen, OH, or C₁₋₇alkoxy, and cycloalkyl and heterocycloalkyl are optionally substituted with 1-4 independently selected C₁₋₇alkyl, halogen, or OH;
or two R₆ together form oxo (═O);
or two Re together form C₁₋₇alkylene to form a ring;
each R′ is hydrogen or C₁₋₇alkyl;
each R₇ is independently halo, C₁₋₇alkyl, C₁₋₇haloalkyl, C₁₋₇alkoxy, or C₁₋₇haloalkoxy, each Rₐ is independently H, C₁₋₇alkyl, C₁₋₇haloalkyl, or C₃₋₈cycloalkyl; and
each R_{b} is independently H, C₁₋₇alkyl, C₁₋₇haloalkyl, or C₃₋₈cycloalkyl.

3. The compound of claim 1 or 2, wherein the compound is of formula (Ia), 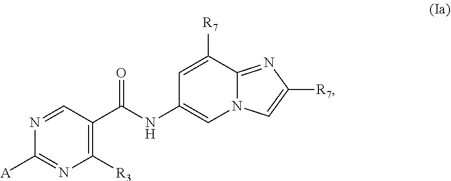
or a pharmaceutically acceptable salt, solvate, or prodrug thereof.

4. The compound of claim 3, wherein A is saturated or partially unsaturated mono- or bi-cyclic 4- to 11-membered nitrogen-containing heterocycloalkyl, and wherein A is attached through a nitrogen of the heterocycloalkyl.

5. The compound of claim 1 or 2, wherein the compound is of formula (Ib), 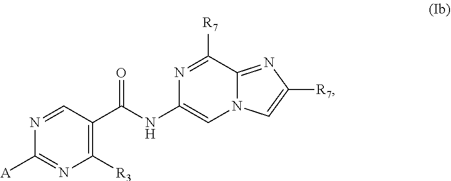
or a pharmaceutically acceptable salt, solvate, or prodrug thereof.

6. The compound of claim 5, wherein A is saturated or partially unsaturated mono- or bi-cyclic 4- to 11-membered nitrogen-containing heterocycloalkyl, and wherein A is attached through a nitrogen of the heterocycloalkyl.

7. The compound of claim 1 or 2, wherein the compound is of formula (Ic) 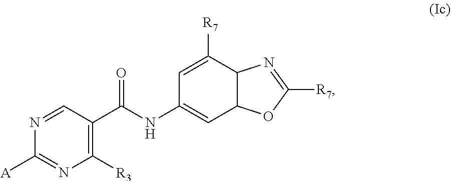
or a pharmaceutically acceptable salt, solvate, or prodrug thereof.

8. The compound of claim 7, wherein A is saturated or partially unsaturated mono- or bi-cyclic 4- to 11-membered nitrogen-containing heterocycloalkyl, and wherein A is attached through a nitrogen of the heterocycloalkyl.

9. The compound of claim 1 or 2, wherein the compound is of formula (Ic) 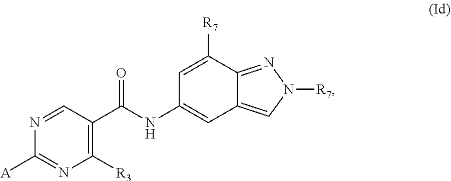
or a pharmaceutically acceptable salt, solvate, or prodrug thereof.

10. The compound of claim 9, wherein A is saturated or partially unsaturated mono- or bi-cyclic 4- to 11-membered nitrogen-containing heterocycloalkyl, and wherein A is attached through a nitrogen of the heterocycloalkyl.

11. The compound of any one of claims 1-10, wherein:
A is 4-11 membered nitrogen-containing heterocycloalkyl, wherein the nitrogen-containing heterocycloalkyl comprises 1 or 2 nitrogen ring atoms and 0-2 additional ring heteroatoms selected from O and S, and is optionally substituted with 1, 2, 3, or 4 R₆;
each R₆ is independently C₁₋₇alkyl, —(CH₂)₀₋₃—NRₐR_{b}, C₃₋₈cycloalkyl, heterocycloalkyl and or two R₆ together form C₁₋₇alkylene to form a ring;
each Rₐ is independently H or C₁₋₇alkyl; and
each R₆ is independently H or C₁₋₇alkyl.

12. The compound of any one of claims 1-11, wherein each R₆ is independently C₁₋₇ alkyl, heterocycloalkyl, or two R₆ together form C₁₋₇alkylene to form a ring.

13. The compound of any one of claims 1-12, wherein each R₆ is independently methyl, ethyl, isopropyl, methoxy-azetidinyl, or pyrrolidinyl, or two Re together form ethylene or propylene to form a ring.

14. The compound of any one of claims 1-13, wherein each R₆ is independently —(CH₂)₀₋₃—NRₐR_{b}.

15. The compound of any one of claims 1-14, wherein Rₐ is H and R_{b} is C₁₋₇alkyl.

16. The compound of any one of claims 1-14, wherein Rₐ and Re are each independently C₁₋₇alkyl.

17. The compound of any one of claims 1-14, wherein Rₐ and Re are each independently H.

18. The compound of any one of claims 1-17, wherein R_{b} is —NH(CH₃), —N(CH₃)₂, —NH(CH₂CH₃), —CH₂NH(CH₃), or —CH₂N(CH₃)₂.

19. The compound of any one of claims 1-18, wherein R₆ is —NH(CH₃).

20. The compound of any one of claims 1-18, wherein R₆ is —N(CH₃)₂.

21. The compound of any one of claims 1-18, wherein R₆ is —NH(CH₂CH₃).

22. The compound of any one of claims 1-18, wherein Re is —CH₂NH(CH₃).

23. The compound of any one of claims 1-18, wherein R₆ is —CH₂N(CH₃)₂.

24. The compound of any one of claims 1-23, wherein
A is 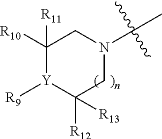
Y is absent, N or CH;
R₉ is hydrogen, C₁₋₇alkyl, or (CH₂)ₘ—NR₁₄R₁₅,
R₁₀ is hydrogen or C₁₋₇alkyl optionally substituted with one or more halo;
R₁₁ is hydrogen or C₁₋₇alkyl optionally substituted with one or more halo;
R₁₂ is hydrogen or C₁₋₇alkyl optionally substituted with one or more halo;
R₁₃ is hydrogen or C₁₋₇alkyl optionally substituted with one or more halo;
each R₁₄ and R₁₅ are independently hydrogen, C₁₋₇alkyl and C₃₋₈cycloalkyl;
n is 0, 1 or 2;
m is 0, 1 or 2;
or R₉ and R₁₀ together form C₁₋₇alkylene to form a ring;
or R₉ and R₁₂ together form C₁₋₇alkylene to form a ring;
or R₁₀ and R₁₁ together form C₂₋₇alkylene to form a ring or 4- to 6-membered heterocycloalkyl optionally substituted with C₁₋₇alkyl;
or R₁₀ and R₁₂ together form C₁₋₇alkylene to form a ring or 4- to 6-membered heterocycloalkyl optionally substituted with C₁₋₇alkyl;
or R₁₀ and R₁₄ together form C₁₋₇alkylene to form a ring;
or R₁₂ and R₁₅ together form C₂₋₇alkylene to form a ring;
or R₁₂ and R₁₄ together form C₁₋₇alkylene to form a ring;
or R₁₄ and R₁₅ together form C₂₋₇alkylene to form a ring.

25. The compound of claim 24, wherein Y is N.

26. The compound of claim 24, wherein Y is CH and R₉ is (CH₂)ₘ—NR₁₄R₁₅.

27. The compound of any one of claims 24-26, wherein R₉ is —NH(CH₃), —N(CH₃)₂, —NH(CH₂CH₃), —CH₂NH(CH₃), or —CH₂N(CH₃)₂.

28. The compound of any one of claims 24-27, wherein n is 1.

29. The compound of any one of claims 24-28, wherein R₉ is hydrogen, pyrrolidinyl, or methoxy-azetidinyl.

30. The compound of any one of claims 24-29, wherein R₁₀ is hydrogen, methyl, ethyl or isopropyl.

31. The compound of any one of claims 24-30, wherein R₁₁ is hydrogen or methyl.

32. The compound of any one of claims 24-31, wherein R₁₂ is hydrogen or methyl.

33. The compound of any one of claims 24-32, wherein R₁₃ is hydrogen.

34. The compound of any one of claims 24-33, wherein R₉ and R₁₀ together form propylene to form a ring.

35. The compound of any one of claims 24-34, wherein R₁₀ and R₁₁ together form ethylene to form a ring.

36. The compound of any one of claims 24-35, wherein R₁₄ and R₁₅ together form propylene or butylene to form a ring.

37. The compound of any one of claims 1-36, wherein A is 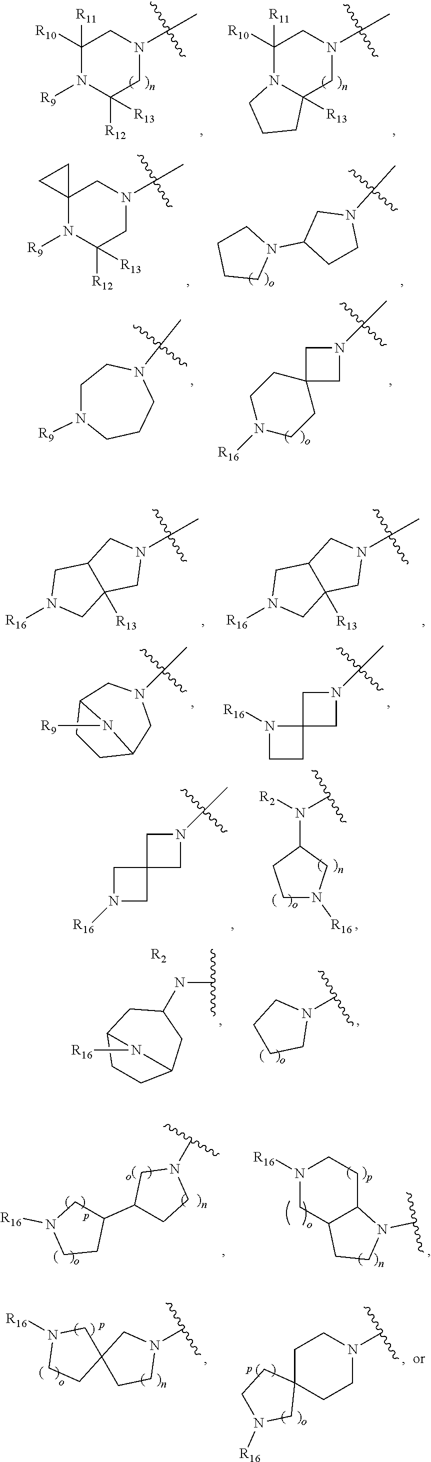 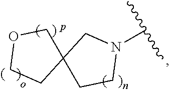
wherein R₉, R₁₀, R₁₁, R₁₂, and R₁₃ are as defined in any one of the preceding claims; each R₁ is independently hydrogen or C₁₋₇alkyl; each p is 0, 1, or 2; each o is 0, 1, or 2; and each A is optionally substituted with one or two Re.

38. The compound of any one of claims 1-36, wherein A is 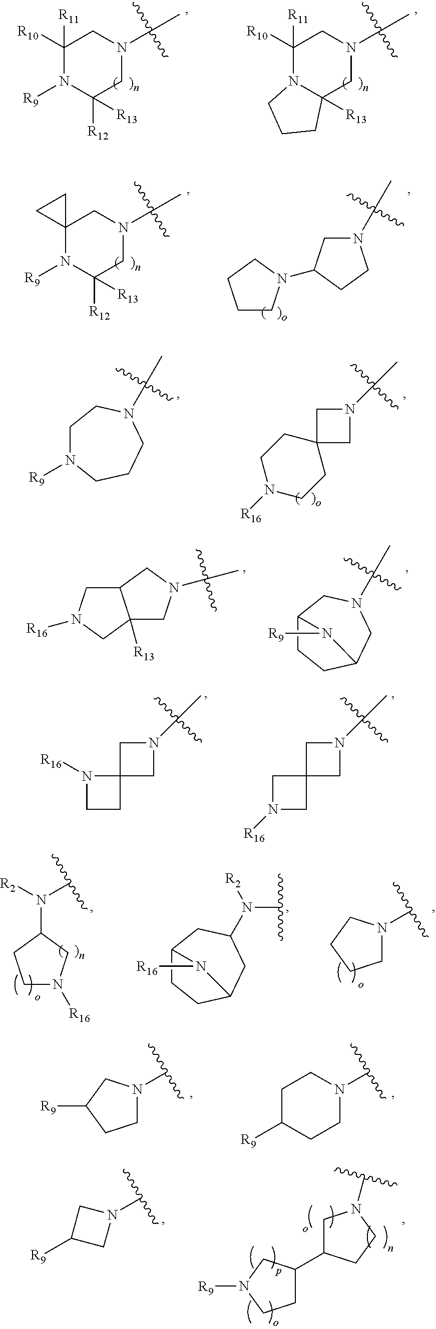 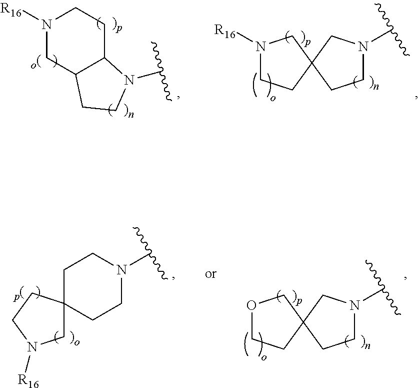
wherein R₉, R₁₀, R₁₁, R₁₂, and R₁₃ are as defined in any one of the preceding claims; each R¹⁶ is independently hydrogen or C₁₋₇alkyl; each p is 0, 1, or 2; each o is 0, 1, or 2; and each A is optionally substituted with one or two R₆.

39. The compound of any one of claims 1-36, wherein A is piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl, wherein piperazinyl, diazepanyl, octahydropyrrolopyrazinyl, diazaspirooctanyl, pyrrolidinyl, octahydropyrrolopyrroyl, diazaspirononanyl, diazaspiroheptanyl, or diazabicyclooctanyl are each optionally substituted with 1, 2, 3, or 4 R₆.

40. The compound of any one of claims 1-36, wherein A is NR₁R₂.

41. The compound of any one of claims 1-36, wherein A is

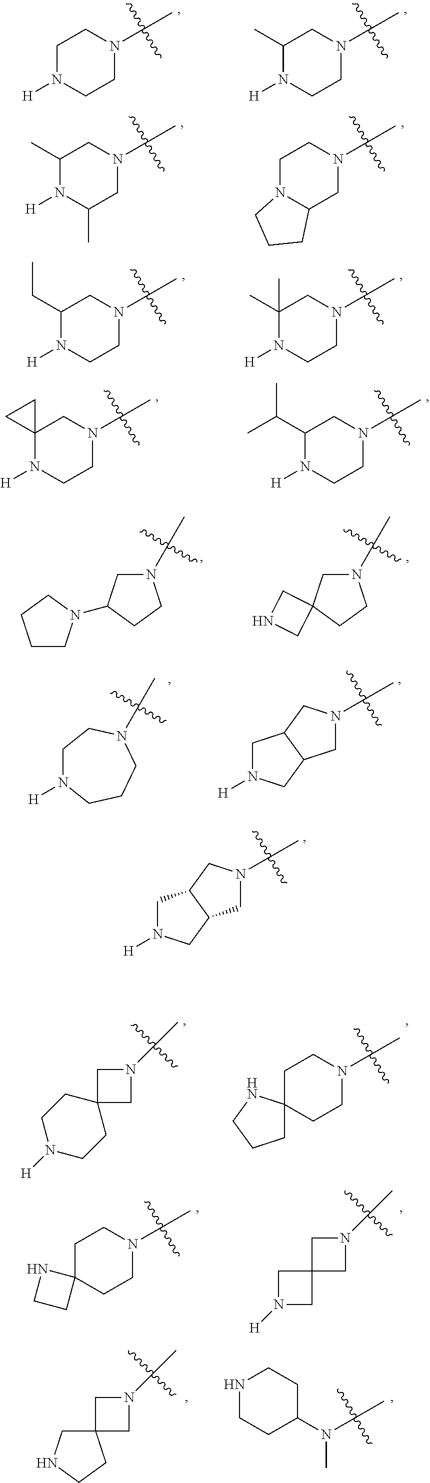

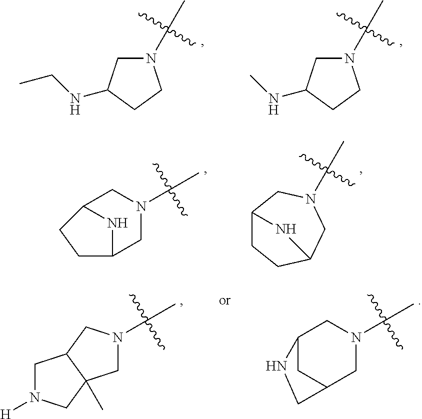

42. The compound of any one of claims 1-36, wherein A is

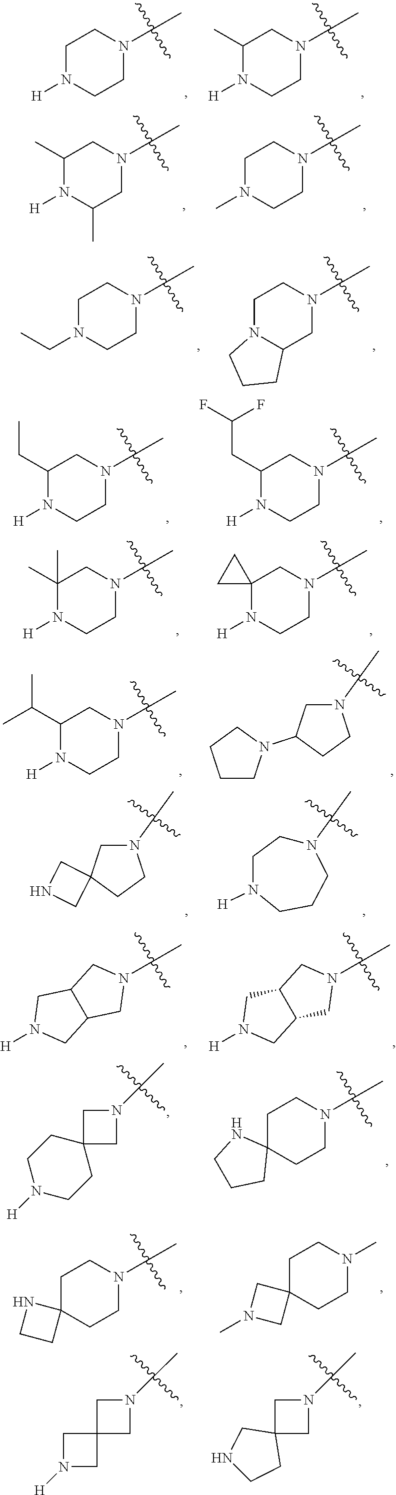

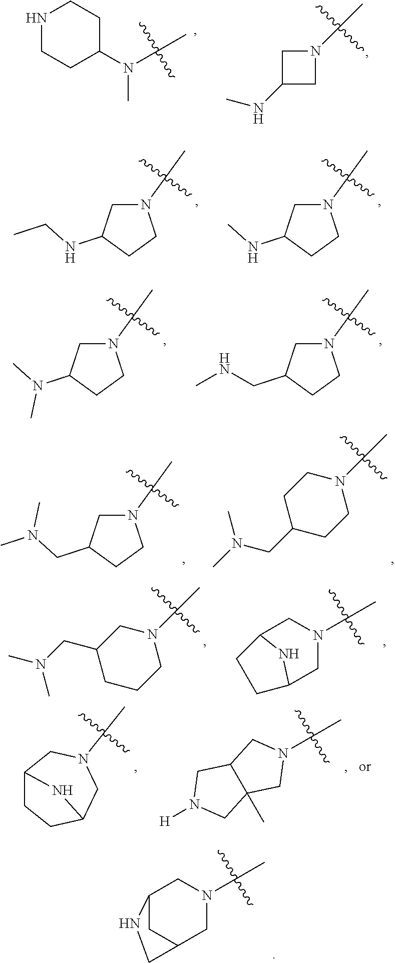

43. The compound of any one of claims 1-36, wherein A is

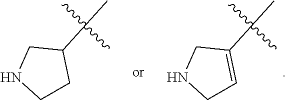 [SMILES: *C1CCNC1 ; *C1=CCNC1]

44. The compound of any one of claims 1-43, wherein R₃ is OR₅.

45. The compound of any one of claims 1-43, wherein R₃ is methoxy, ethoxy, or n-propoxy.

46. The compound of any one of claims 1-43, wherein R₃ is methoxy.

47. The compound of any one of claims 1-43, wherein R₃ is ethoxy.

48. The compound of any one of claims 1-47, wherein R₄ is a bicyclic 9-membered heteroaryl comprising 2 heteroatoms independently selected from N and O, wherein R₄ can be optionally substituted with 1, 2, or 3 R₇.

49. The compound of any one of claims 1-47, wherein Rₐ is a bicyclic 9-membered heteroaryl comprising 2 heteroatoms independently selected from N and O, wherein R₄ is substituted with 1 or 2 R₇.

50. The compound of any one of claims 1-47, wherein R₄ is

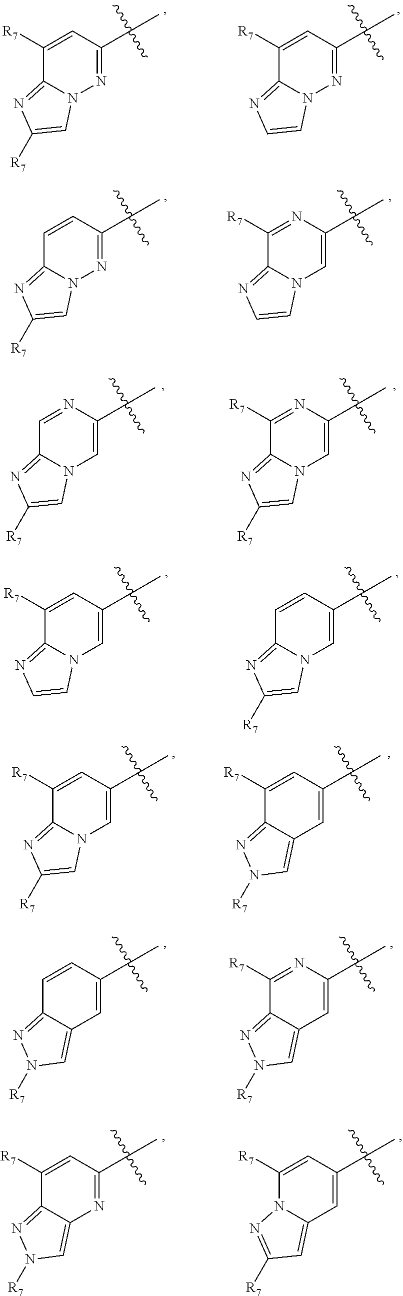

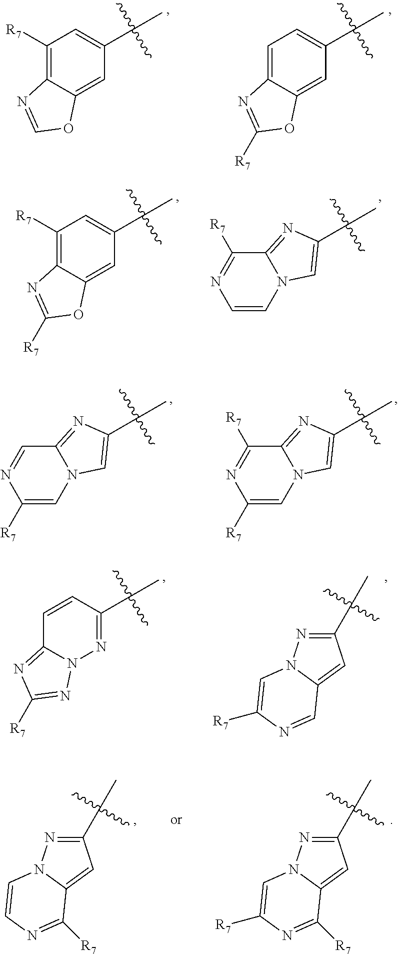

51. The compound of any one of claims 1-47, wherein R₄ is

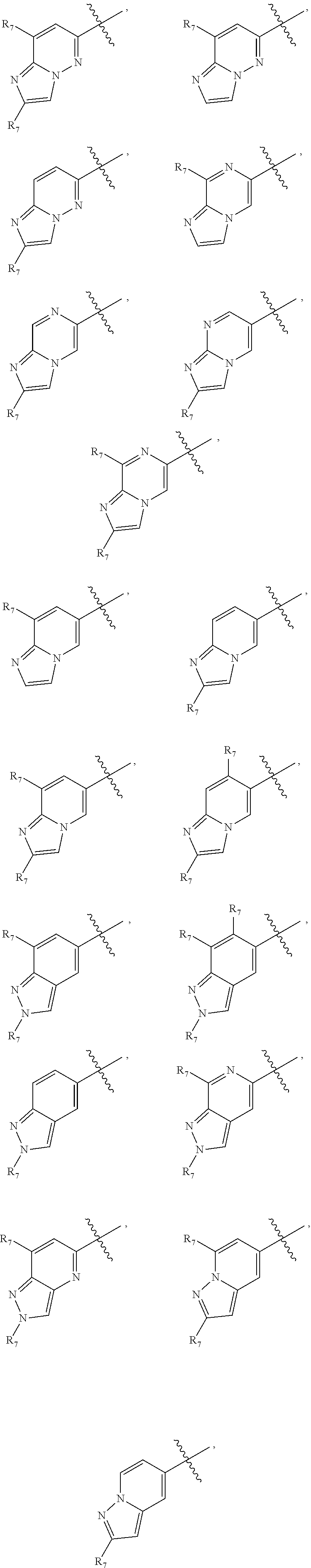

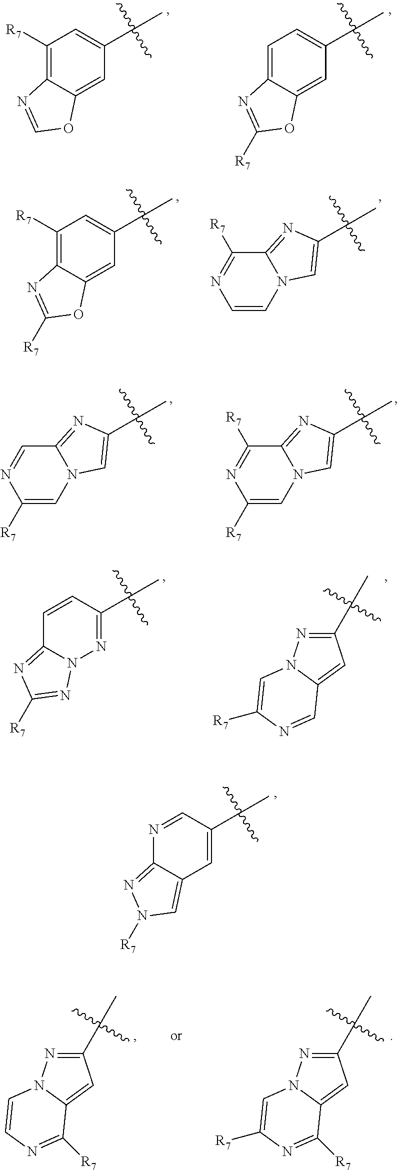

52. The compound of any one of claims 1-47, wherein R₄ is

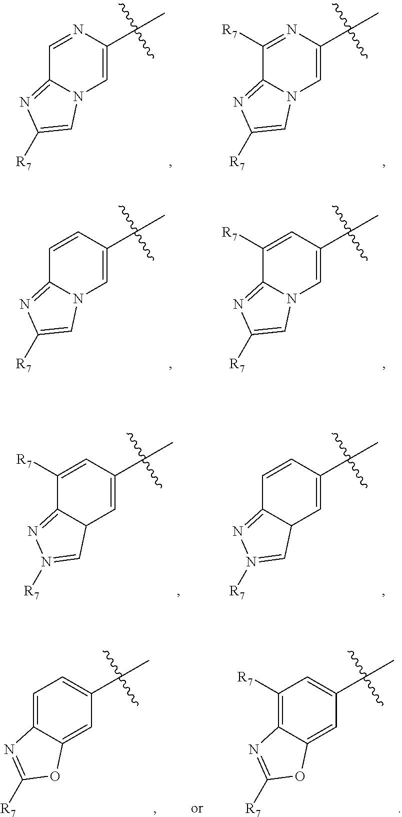

53. The compound of any one of claims 1-47, wherein R₄ is selected from imidazo[1,2-a]pyrazine, benzo[d]oxazole, or imidazo[1,2-a]pyrazine, wherein imidazo[1,2-a]pyrazine, benzo[d]oxazole, or imidazo[1,2-a]pyrazine are each optionally substituted with 1, 2, 3 or 4 R₇.

54. The compound of any one of claims 1-52, wherein R₄ is

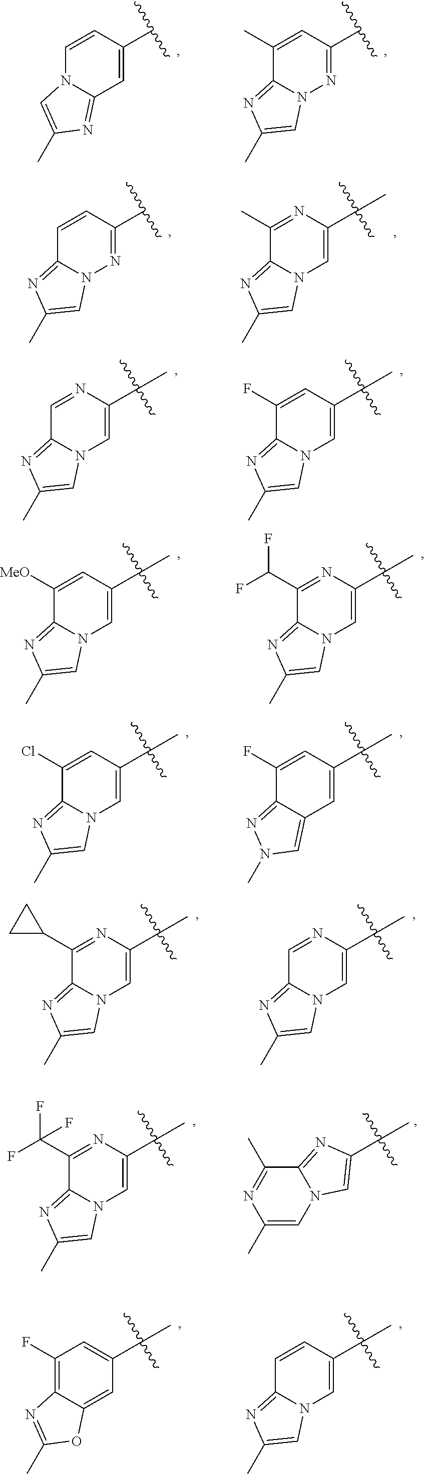

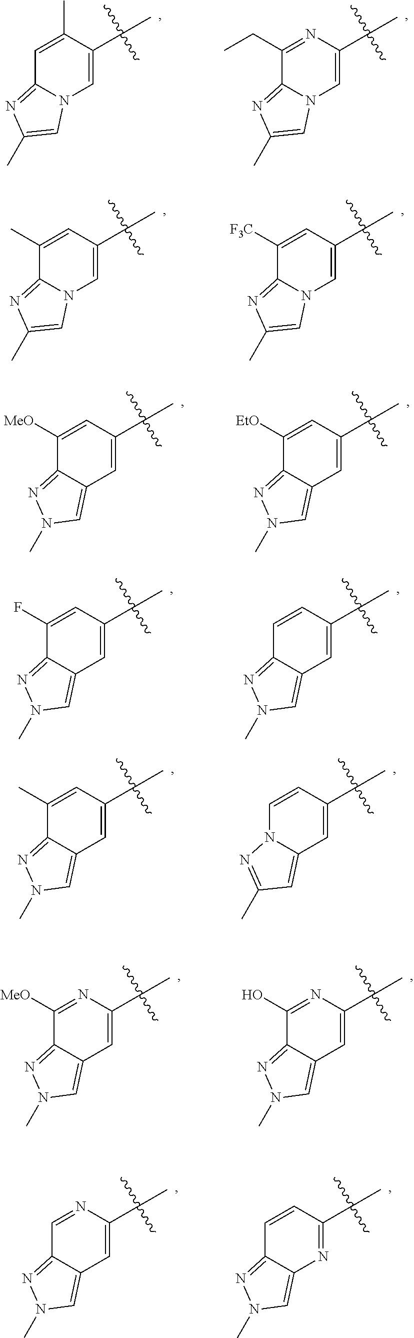

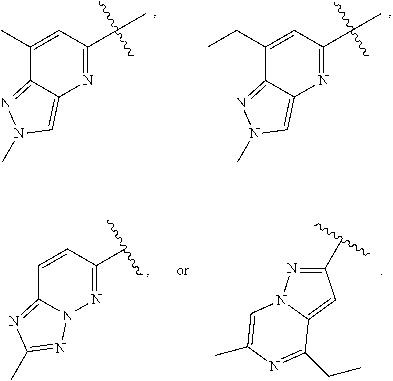 [SMILES: *c1cc(C)c2nn(C)cc2n1 ; *c1cc(CC)c2nn(C)cc2n1 ; *c1ccc2nc(C)nn2n1 ; *c1cc2c(CC)nc(C)cn2n1]

55. The compound of any one of claims 1-52, wherein R₄ is

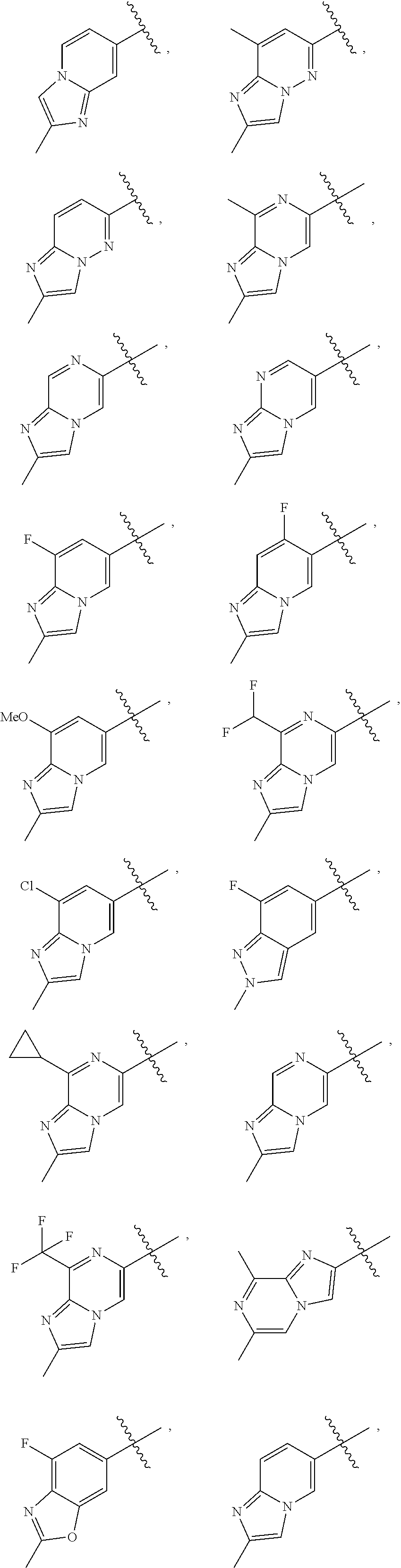

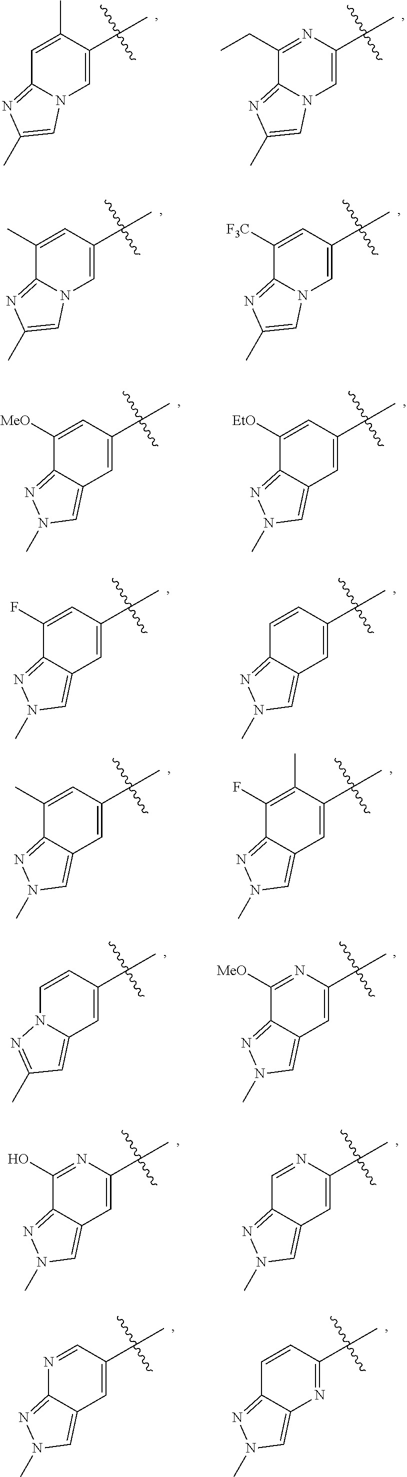

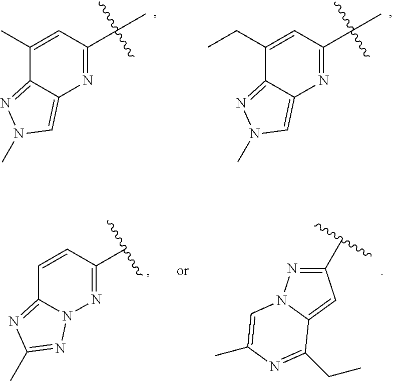 [SMILES: *c1cc(C)c2nn(C)cc2n1 ; *c1cc(CC)c2nn(C)cc2n1 ; *c1ccc2nc(C)nn2n1 ; *c1cc2c(CC)nc(C)cn2n1]

56. The compound of any one of claims 1-55, wherein R₄ is

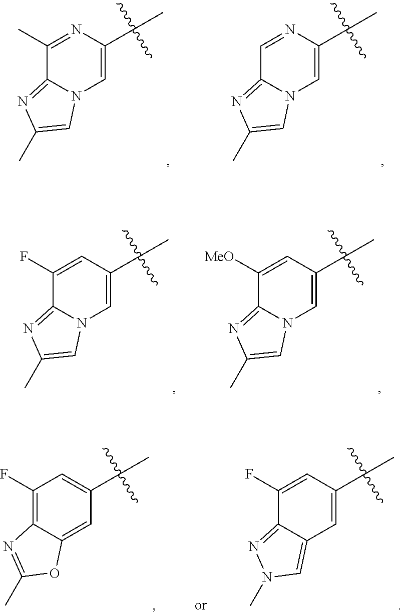 [SMILES: *c1cn2cc(C)nc2c(C)n1 ; *c1cn2cc(C)nc2cn1 ; *c1cc(F)c2nc(C)cn2c1 ; *c1cc(OC)c2nc(C)cn2c1 ; *c1cc(F)c2nc(C)oc2c1 ; *c1cc(F)c2nn(C)cc2c1]

57. The compound of any one of claims 1-56, selected from a compound of Table 1.

58. The compound of any one of claims 1-57, or a pharmaceutically acceptable salt, solvate, or prodrug thereof for use as a small molecule splicing modulator.

59. A pharmaceutical composition comprising a compound of any one of claims 1-58, or a pharmaceutically acceptable salt, solvate, or prodrug thereof and one or more pharmaceutically acceptable excipients.

60. A method of treating a disorder related to a nucleotide repeat expansion, comprising administering to the subject a therapeutically effective amount of a compound of any one of claims 1-58 or a pharmaceutical composition of claim 59.

61. The method of claim 60, wherein the nucleotide repeat expansion comprises a nucleotide sequence repeated two or more times, wherein the nucleotide sequence is selected from the group consisting of CAG, CAG/CTG, GCG, GCN, CGG, CCG, CCCCGCCCCGCG, GCA, GGGGCC, CTG, GAA, ATTCT, TGGAA, GGCCTG, AAGGG, CCCTCT, ATTTT/ATTTC, and CCCTCT.

62. The method of claim 60, wherein the nucleotide repeat expansion comprises a trinucleotide sequence repeated two or more times, wherein the trinucleotide sequence is selected from the group consisting of CAG, CTG, CGG, and GCN.

63. A method of treating a disease in a subject in need thereof, the method comprising administering to the subject a therapeutically effective amount of a compound of any one of claims 1-58 or a pharmaceutical composition of claim 59, wherein the disease is selected from the group consisting of Dentatorubropallidoluysian atrophy, Huntington's disease, Spinal and bulbar muscular atrophy, SCA1 (Spinocerebellar ataxia Type 1), SCA2 (Spinocerebellar ataxia Type 2), SCA3 (Spinocerebellar ataxia Type 3 or Machado-Joseph disease), SCA6 (Spinocerebellar ataxia Type 6), SCA7 (Spinocerebellar ataxia Type 7), SCA12 (Spinocerebellar ataxia Type 12), SCA17 (Spinocerebellar ataxia Type 17), FRAXA (Fragile X syndrome), FXTAS (Fragile X-associated tremor/ataxia syndrome), FRAXE (Fragile XE mental retardation), Baratela-Scott syndrome, FRDA (Friedreich's ataxia), DM1 (Myotonic dystrophy Type 1), DM2 (Myotonic dystrophy Type 2) SCA8 (Spinocerebellar ataxia Type 8), Fuchs endothelial corneal dystrophy, Desbuquois dysplasia, amyotrophic lateral sclerosis, frontotemporal dementia.

64. A method of treating a disease in a subject in need thereof, the method comprising administering to the subject a therapeutically effective amount of a compound of any one of claims 1-58 or a pharmaceutical composition of claim 59, wherein the disease is Huntington's disease.

65. A method of treating a disease in a subject in need thereof, the method comprising administering to the subject a therapeutically effective amount of a compound of any one of claims 1-58 or a pharmaceutical composition of claim 59, wherein the disease is Myotonic dystrophy.

66. A method of treating a disease in a subject in need thereof, the method comprising administering to the subject a therapeutically effective amount of a compound of any one of claims 1-58 or a pharmaceutical composition of claim 59, wherein the disease is FRAXA (Fragile X syndrome), FXTAS (Fragile X-associated tremor/ataxia syndrome), or FRAXE (Fragile XE mental retardation).
